(19) United States
(12) Patent Application Publication
(10) Pub. No.: US 2023/0150920 A1
(43) Pub. Date: May 18, 2023

(54) SALTS AND SOLID FORMS OF 4-BROMO-2,5-DIMETHOXYPHENETHYLAMINE

(71) Applicant: Terran Biosciences, Inc., New York, NY (US)

(72) Inventors: Matthew DUNCTON, San Bruno, CA (US); Samuel CLARK, New York, NY (US)

(21) Appl. No.: 17/987,855

(22) Filed: Nov. 15, 2022

Related U.S. Application Data
(60) Provisional application No. 63/357,616, filed on Jun. 30, 2022.
(60) Provisional application No. 63/326,790, filed on Apr. 1, 2022.
(60) Provisional application No. 63/326,786, filed on Apr. 1, 2022.
(60) Provisional application No. 63/320,653, filed on Mar. 16, 2022.
(60) Provisional application No. 63/320,650, filed on Mar. 16, 2022.
(60) Provisional application No. 63/316,999, filed on Mar. 5, 2022.
(60) Provisional application No. 63/311,877, filed on Feb. 18, 2022.
(60) Provisional application No. 63/305,638, filed on Feb. 1, 2022.
(60) Provisional application No. 63/279,653, filed on Nov. 15, 2021.
(60) Provisional application No. 63/279,657, filed on Nov. 15, 2021.

(51) Int. Cl. C07C 215/52

(52) U.S. Cl. CPC C07C 215/52; C07B 2200/13

(57) ABSTRACT

Disclosed herein are salts and solid forms of 4-bromo-2,5-dimethoxyphenethylamine. The solid form may be a salt and/or a crystalline form of 4-bromo-2,5-dimethoxyphenethylamine, such as a polymorph of 4-bromo-2,5-dimethoxyphenethylamine or a salt thereof. Also disclosed are methods for making the salts and solid forms and methods for administering the solid forms. The salts and solid forms of 4-bromo-2,5-dimethoxyphenethylamine are useful for treating neurological disease and/or a psychiatric disorder in a subject.

### CROSS-REFERENCES TO RELATED APPLICATIONS

This application claims priority to U.S. Provisional Application No. 63/279,653 filed Nov. 15, 2021; U.S. Provisional Application No. 63/305,638 filed Feb. 1, 2022; U.S. Provisional Application No. 63/311,877 filed Feb. 18, 2022; U.S. Provisional Application No. 63/320,653 filed Mar. 16, 2022; U.S. Provisional Application No. 63/326,786 filed Apr. 1, 2022, U.S. Provisional Application No. 63/357,616 filed Jun. 30, 2022; U.S. Provisional Application No. 63/316,999 filed Mar. 5, 2022; U.S. Provisional Application No. 63/320,650 filed Mar. 16, 2022; U.S. Provisional Application No. 63/326,790 filed Apr. 1, 2022; and U.S. Provisional Application No. 63/279,657 filed Nov. 15, 2021, which are incorporated herein by reference in their entirety to the full extent permitted by law.

### FIELD OF THE INVENTION

The present disclosure relates to salts and solid forms of 4-bromo-2,5-dimethoxyphenethylamine, processes for their preparation and their use in the manufacture of a medicament for treating patients. The disclosure is also directed to pharmaceutical compositions containing at least one salt or solid form of 4-bromo-2,5-dimethoxyphenethylamine and to the therapeutic and/or prophylactic use of such salts and solid forms and compositions.

### SUMMARY

Disclosed herein are salts and solid forms of 4-bromo-2,5-dimethoxyphenethylamine (compound 1), crystalline forms, and polymorphs of same. Also disclosed are methods for making the salts and solid forms and methods for using the salts and solid forms of 4-bromo-2,5-dimethoxyphenethylamine. In some embodiments, the solid form of 4-bromo-2,5-dimethoxyphenethylamine is a polymorph of the free base form of the compound. In other embodiments, the solid form of 4-bromo-2,5-dimethoxyphenethylamine is a salt, and maybe a polymorph of the salt. The salt may be formed from an acid selected from galactaric (mucic) acid, naphthalene-1,5-disulfonic acid, citric acid, sulfuric acid, d-glucuronic acid, ethane-1,2-disulfonic acid, lactobionic acid, p-toluenesulfonic acid, D-glucoheptonic acid, thiocyanic acid, (−)-L-pyroglutamic acid, methanesulfonic acid, L-malic acid, dodecylsulfuric acid, hippuric acid, naphthalene-2-sulfonic acid, D-gluconic acid, benzenesulfonic acid, D,L-lactic acid, oxalic acid, oleic acid, glycerophosphoric acid, succinic acid, ethanesulfonic acid 2-hydroxy, glutaric acid, L-aspartic acid, cinnamic acid, maleic acid, adipic acid, phosphoric acid, sebacic acid, ethanesulfonic acid, (+)-camphoric acid, glutamic acid, acetic acid, or a combination thereof.

In any embodiments, the solid form may be a crystalline solid. The crystalline solid may be substantially a single form, such as a polymorph form. And the polymorph may be selected to have one or more desired properties, particularly improved properties, such as physical properties, chemical properties, pharmacokinetic properties, or a combination thereof. The one or more desired properties may comprise melting point, glass transition temperature, flowability, thermal stability, mechanical stability, shelf life, stability against polymorphic transition, hygroscopic properties, solubility in water and/or organic solvents, reactivity, compatibility with excipients and/or delivery vehicles, bioavailability, absorption, distribution, metabolism, excretion, toxicity including cytotoxicity, dissolution rate, half-life, or a combination thereof.

In any embodiments, the solid form of 4-bromo-2,5-dimethoxyphenethylamine salt may be a solvate, such as a hydrate.

Furthermore, disclosed herein are novel solid forms of 4-bromo-2,5-dimethoxyphenethylamine hydrochloride (2C-B hydrochloride or 2C-B.HCl). The solid form of 4-bromo-2,5-dimethoxyphenethylamine.HCl may have at least one improved property compared to amorphous 4-bromo-2,5-dimethoxyphenethylamine hydrochloride.

Also disclosed herein is a solid form of 4-bromo-2,5-dimethoxyphenethylamine hydrochloride that is made by the method described in Example 1. The solid form of 4-bromo-2,5-dimethoxyphenethylamine hydrochloride made by the disclosed method may have at least one improved property compared to amorphous 4-bromo-2,5-dimethoxyphenethylamine hydrochloride.

In any embodiments, the at least one improved property of the solid form of 4-bromo-2,5-dimethoxyphenethylamine hydrochloride may comprise a physical property, chemical property, pharmacokinetic property, or a combination thereof. In some embodiments, the at least one improved property comprises a melting point, glass transition temperature, flowability, thermal stability, shelf life, stability against polymorphic transition, hygroscopic properties, solubility in water and/or organic solvents, reactivity, compatibility with excipients and/or delivery vehicles, bioavailability, absorption, distribution, metabolism, excretion, toxicity including cytotoxicity, dissolution rate, half-life, or a combination thereof, that is improved compared to an amorphous sample of 4-bromo-2,5-dimethoxyphenethylamine hydrochloride.

In any embodiments, the solid form of 4-bromo-2,5-dimethoxyphenethylamine hydrochloride may be a solvate, such as a hydrate.

Also disclosed herein are embodiments, of a pharmaceutical composition, comprising a salt or solid form of a disclosed compound, and a pharmaceutically acceptable excipient.

A method for administering the salt or solid form of 4-bromo-2,5-dimethoxyphenethylamine also is disclosed herein. In some embodiments, the method comprises administering to a subject an effective amount of a salt or solid form of 4-bromo-2,5-dimethoxyphenethylamine, or a pharmaceutical composition thereof. In some embodiments, the subject is suffering from a neurological disease or a psychiatric disorder, or both, such as a neurodegenerative disorder. The neurological disorder or psychiatric disorder, or both, may comprise depression, addiction, anxiety, or a post-traumatic stress disorder, and/or the neurological disorder or psychiatric disorder, or both, may comprise treatment resistant depression, suicidal ideation, major depressive disorder, bipolar disorder, schizophrenia, or substance use disorder. In some embodiments, the neurological disorder or psychiatric disorder, or both, comprises stroke, traumatic brain injury, or a combination thereof.

In any embodiments, administering the salt or solid form of compound 1 comprises oral, parenteral, or topical administration. In certain embodiments, oral administration is used, but in other particular embodiments, administration is by injection, inhalation, intraocular, intravaginal, intrarectal or transdermal routes.

In some embodiments, the A solid form of 4-bromo-2,5-dimethoxyphenethylamine hydrochloride has at least one improved property compared to amorphous 4-bromo-2,5-dimethoxyphenethylamine hydrochloride.

In another embodiment, the solid form of 4-bromo-2,5-dimethoxyphenethylamine hydrochloride is made by the method described in Example 1.

In yet another embodiment, the solid form of 4-bromo-2,5-dimethoxyphenethylamine hydrochloride has at least one improved property compared to amorphous 4-bromo-2,5-dimethoxyphenethylamine hydrochloride.

In some embodiments, the solid form of 4-bromo-2,5-dimethoxyphenethylamine hydrochloride has at least one improved property compared to amorphous 4-bromo-2,5-dimethoxyphenethylamine hydrochloride, wherein the at least one improved property comprises a physical property, chemical property, pharmacokinetic property, or a combination thereof.

In yet other embodiments, the solid form of 4-bromo-2,5-dimethoxyphenethylamine hydrochloride has at least one improved property compared to amorphous 4-bromo-2,5-dimethoxyphenethylamine hydrochloride, wherein the at least one improved property comprise melting point, glass transition temperature, flowability, thermal stability, shelf life, stability against polymorphic transition, hygroscopic properties, solubility in water and/or organic solvents, reactivity, compatibility with excipients and/or delivery vehicles, bioavailability, absorption, distribution, metabolism, excretion, toxicity including cytotoxicity, dissolution rate, half-life, or a combination thereof.

In other embodiments, the solid form of 4-bromo-2,5-dimethoxyphenethylamine hydrochloride is a hydrate.

In some embodiments, the pharmaceutical composition, comprise a solid form of 4-bromo-2,5-dimethoxyphenethylamine hydrochloride and a pharmaceutically acceptable excipient

In some embodiments, an effective amount of a solid form of 4-bromo-2,5-dimethoxyphenethylamine hydrochloride, or a pharmaceutical composition comprising 4-bromo-2,5-dimethoxyphenethylamine hydrochloride is administered to a subject in need thereof.

In some embodiments, the subject has a neurological disease or a psychiatric disorder, or both.

In some embodiments, the neurological disorder is a neurodegenerative disorder.

In some embodiments, the neurological disorder or psychiatric disorder, or both, comprises depression, addiction, anxiety, or a post-traumatic stress disorder.

In some embodiments, the neurological disorder or psychiatric disorder, or both, comprises treatment resistant depression, suicidal ideation, major depressive disorder, bipolar disorder, schizophrenia, or substance use disorder.

In some embodiments, the neurological disorder or psychiatric disorder, or both, comprises stroke, traumatic brain injury, or a combination thereof.

In some embodiments the administering comprises oral, parenteral, or topical administration.

In some embodiments, the administering comprises administering by injection, inhalation, intraocular, intravaginal, intrarectal or transdermal routes.

In some embodiments, the solid form of 4-bromo-2,5-dimethoxyphenethylamine hydrochloride is Form A.

In some embodiments, the solid form of 4-bromo-2,5-dimethoxyphenethylamine hydrochloride is Form B.

In some embodiments, the solid form of 4-bromo-2,5-dimethoxyphenethylamine hydrochloride is Form C.

In some embodiments, the salt or solid form of 4-bromo-2,5-dimethoxyphenethylamine is not 4-bromo-2,5-dimethoxyphenethylamine hydrochloride.

In some embodiments, the solid form of 2C-B is a salt.

In some embodiments, the salt is formed from an acid selected from galactaric (mucic) acid, naphthalene-1,5-disulfonic acid, citric acid, sulfuric acid, d-glucuronic acid, ethane-1,2-disulfonic acid, lactobionic acid, p-toluenesulfonic acid, D-glucoheptonic acid, thiocyanic acid, (−)-L-pyroglutamic acid, methanesulfonic acid, L-malic acid, dodecylsulfuric acid, hippuric acid, naphthalene-2-sulfonic acid, D-gluconic acid, benzenesulfonic acid, D,L-lactic acid, oxalic acid, oleic acid, glycerophosphoric acid, succinic acid, ethanesulfonic acid 2-hydroxy, glutaric acid, L-aspartic acid, cinnamic acid, maleic acid, adipic acid, phosphoric acid, sebacic acid, ethanesulfonic acid, (+)-camphoric acid, glutamic acid, acetic acid, or a combination thereof.

In some embodiments, the 2C-B salt, wherein the 2C-B salt is formed using a stoichiometric ratio of acid to 4-bromo-2,5-dimethoxyphenethylamine from about 0.4 molar equivalent to about 2.2 molar equivalents of the acid.

In some embodiments, the 2C-B salt, wherein the 2C-B salt is formed using a stoichiometric ratio of acid to 4-bromo-2,5-dimethoxyphenethylamine from about 0.5 molar equivalent to about 2 molar equivalents of the acid.

In some embodiments, the 2C-B salt, wherein the 2C-B salt is formed using a stoichiometric ratio of acid to 4-bromo-2,5-dimethoxyphenethylamine from about 0.5, 1 and 2 molar equivalents of the acid.

In some embodiments, the solid form of 2C-B is a free base form of 4-bromo-2,5-dimethoxyphenethylamine.

In some embodiments, the salt or solid form of 2C-B is a crystalline solid.

In some embodiments, the salt or solid form of 2C-B is crystalline solid and is substantially a single polymorph.

In some embodiments, the salt or solid form of 2C-B is a polymorph which is selected to have one or more desired properties.

In some embodiments, the salt or solid form of 2C-B has one or more desired properties selected from physical properties, chemical properties, pharmacokinetic properties, or a combination thereof.

In some embodiments, the salt or solid form of 2C-B is a hydrate.

In some embodiments, the salt or solid form of 2C-B has the one or more desired properties comprise melting point, glass transition temperature, flowability, thermal stability, shelf life, stability against polymorphic transition, hygroscopic properties, solubility in water and/or organic solvents, reactivity, compatibility with excipients and/or delivery vehicles, bioavailability, absorption, distribution, metabolism, excretion, toxicity including cytotoxicity, dissolution rate, half-life, or a combination thereof.

In some embodiments, the pharmaceutical composition, comprise a salt or solid form of 4-bromo-2,5-dimethoxyphenethylamine and a pharmaceutically acceptable excipient.

In some embodiments, an effective amount of a salt or solid form of 4-bromo-2,5-dimethoxyphenethylamine hydrochloride, or a pharmaceutical composition comprising 4-bromo-2,5-dimethoxyphenethylamine hydrochloride is administered to a subject in need thereof.

In some embodiments, an effective amount of a salt or solid form of 4-bromo-2,5-dimethoxyphenethylamine, or a pharmaceutical composition comprising a salt or solid form of 4-bromo-2,5-dimethoxyphenethylamine is administered to a subject in need thereof.

In some embodiments, the neurological disorder, psychiatric disorder, or both, comprises depression, addiction, anxiety, or a post-traumatic stress disorder.

In some embodiments, the neurological disorder, psychiatric disorder, or both, comprises treatment resistant depression, suicidal ideation, major depressive disorder, bipolar disorder, schizophrenia, or substance use disorder.

In some embodiments, the neurological disorder, psychiatric disorder, or both, comprises stroke, traumatic brain injury, or a combination thereof.

In some embodiments, the 2C-B salt is selected from the aspartate, besylate, citrate esylate, fumarate, gentisate, gluconate, glutamate, glycolate, sulfate, phosphate, xinafoate, lactate, malate, maleate, malonate, mesylate, mucate, succinate, tartrate and tosylate form of 4-bromo-2,5-dimethoxyphenethylamine.

In some embodiments, the 2C-B salt is a solid form.

In some embodiments, the 2C-B salt comprises a crystalline form of the salt.

In some embodiments, the pharmaceutical composition comprises a salt or solid form of 4-bromo-2,5-dimethoxyphenethylamine and a pharmaceutically acceptable excipient.

In some embodiments, an effective amount of a salt or solid form of 4-bromo-2,5-dimethoxyphenethylamine is administered to a subject in need thereof.

In some embodiments, an effective amount of a pharmaceutical composition comprising a salt or solid form of 4-bromo-2,5-dimethoxyphenethylamine is administered to a subject in need thereof.

In some embodiments, the subject has a neurological disorder, a psychiatric disorder, or both.

In some embodiments, the 2C-B Tartrate salt is crystalline polymorphic (Form 1) characterized by two or more, or three XRPD signals selected from the group consisting of 20.3 °2θ, 24.7 °2θ, and 27.2 °2θ (±0.2 °2θ; ±0.1 °2θ; or ±0.0 °2θ; Cu Kα1 radiation).

In some embodiments, the 2C-B Tartrate salt is crystalline polymorphic (Form 1) characterized by two or more, or three XRPD signals selected from the group consisting of 10.3 °2θ, 12.9 °2θ, and 14.2 °2θ (±0.2 °2θ; ±0.1 °2θ; or ±0.0 °2θ; Cu Kα1 radiation).

In some embodiments, the 2C-B Tartrate salt is crystalline polymorphic (Form 1) characterized by two or more, or three XRPD signals selected from the group consisting of 10.3 °2θ, 12.9 °2θ, 14.2 °2θ, 14.4 °2θ and 20.3 °2θ (±0.2 °2θ; ±0.1 °2θ; or ±0.0 °2θ; Cu Kα1 radiation).

In some embodiments, the 2C-B Tartrate salt is crystalline polymorphic (Form 1) characterized by two, three, four, five, six, seven, eight, nine, or ten XRPD signals as shown in Table 70.

In some embodiments, the 2C-B Tartrate salt is crystalline polymorphic (Form 1) characterized by two or more, or three XRPD signals as shown in FIG. 64.

In some embodiments, 2C-B Tartrate (Form 1) has an ¹H NMR spectra as provided in FIG. 93.

In some embodiments, 2C-B Tartrate (Form 1) has an ¹H NMR spectra as provided in FIG. 105.

In some embodiments, 2C-B Tartrate (Form 1) has TGA and DSC profiles as provided in FIG. 122.

In some embodiments, 2C-B Tartrate (Form 1) has TGA and DSC profiles as provided in FIG. 132.

In some embodiments, 2C-B Tartrate (Form 1) has a DVS profile as provided in FIG. 144.

In some embodiments, the 2C-B tartrate salt (Form 1) possess at least one of characteristic as provided in Table 86.

In some embodiments, single crystal structure of 4-bromo-2,5-dimethoxyphenethylamine (2C-B) tartrate is anhydrous with 1:1 2C-B:tartaric acid stoichiometry and formula C₁₀H₁₅BrNO₂.C₄H₅O₆.

In some embodiments, single crystal structure of 4-bromo-2,5-dimethoxyphenethylamine (2C-B) tartrate has unit cell parameters as provided in Table 87A.

In some embodiments, the 2C-B tartrate salt is prepared by the method provided in Example 5.

In some embodiments, the 2C-B salt is prepared by a method as described in Example 5.

In some embodiments, the 2C-B polymorph is prepared by a method as described in Example 4 or Example 5. In some embodiments, the 2C-B tartrate salt is prepared by a method as described in Example 4 or Example 5.

In some embodiments, the present disclosure is directed to a salt of mescaline, wherein the salt is mescaline HCl is a crystalline polymorph characterized by two or more, or three XRPD signals selected from the group consisting of 9.4 °2θ, 13.8 °2θ, and 14.2 °2θ (±0.2 °2θ; ±0.1 °2θ; or ±0.0 °2θ; Cu Kα1 radiation).

In some embodiments, the present disclosure is directed to a salt of mescaline, mescaline HCl is a crystalline polymorph characterized by two or more, or three XRPD signals selected from the group consisting of 9.4 °2θ, 13.8 °2θ, 14.2 °2θ, 15.8 °2θ, and 19.0 °2θ (±0.2 °2θ; ±0.1 °2θ; or ±0.0 °2θ; Cu Kα1 radiation).

In some embodiments, the mescaline HCl is crystalline polymorph characterized by two, three, four, five, six, seven, eight, nine, or ten XRPD signals as shown in Table 91.

In some embodiments, the mescaline HCl is crystalline polymorph characterized by two or more, or three XRPD signals as shown in FIG. 159A-D.

The foregoing and other objects, features, and advantages of the invention will become more apparent from the following detailed description, which proceeds with reference to the accompanying figures.

### BRIEF DESCRIPTION OF THE FIGURES

The patent or application file contains at least one drawing executed in color. Copies of this patent or patent application publication with color drawing(s) will be provided by the Office upon request and payment of the necessary fee.

The accompanying drawings, which are included to provide a further understanding of the present disclosure, are incorporated in and constitute a part of this specification, illustrate aspects of the present disclosure and, together with the detailed description, serve to explain the principles of the present disclosure.

FIG. 1 depicts an XRPD diffractogram of 2C-B.HCl Form A. The XRPD signals observed in this diffractogram are characterized in Table 6. FIG. 2 depicts an XRPD diffractogram of 2C-B.HCl Form A. The XRPD signals observed in this diffractogram are characterized in Table 7.

FIG. 3 depicts an XRPD diffractogram of 2C-B.HCl Form B. The XRPD signals observed in this diffractogram are characterized in Table 8.

FIG. 4 depicts an XRPD diffractogram of 2C-B.HCl Form C. The XRPD signals observed in this diffractogram are characterized in Table 9.

FIG. 5 depicts an XRPD diffractogram of 2C-B free base (Forms 1 and 2) prepared according to Example 1, Table 4. The XRPD signals observed in this diffractogram are characterized in Table 10.

FIG. 6 depicts an XRPD diffractogram of 2C-B free base (Form 1) prepared according to Example 1, Table 4. The XRPD signals observed in this diffractogram are characterized in Table 11.

FIG. 7 depicts an XRPD diffractogram of 2C-B.HCl Form A in a preferred orientation. The XRPD signals observed in this diffractogram are characterized in Table 12.

FIG. 8 depicts an XRPD diffractogram of crystalline 2C-B.besylate (Form 1). The XRPD signals observed in this diffractogram are characterized in Table 19.

FIG. 9 depicts an XRPD diffractogram of crystalline 2C-B.citrate (Form 1). The XRPD signals observed in this diffractogram are characterized in Table 20.

FIG. 10 depicts an XRPD diffractogram of crystalline 2C-B.esylate (Form 1). The XRPD signals observed in this diffractogram are characterized in Table 21.

FIG. 11 depicts an XRPD diffractogram of crystalline 2C-B.fumarate (Form 1). The XRPD signals observed in this diffractogram are characterized in Table 22.

FIG. 12 depicts an XRPD diffractogram of crystalline 2C-B.gentisate. The XRPD signals observed in this diffractogram are characterized in Table 23.

FIG. 13 depicts an XRPD diffractogram of crystalline 2C-B.gluconate.

FIG. 14 depicts an XRPD diffractogram of crystalline 2C-B.glycolate. The XRPD signals observed in this diffractogram are characterized in Table 25.

FIG. 15 depicts an XRPD diffractogram of crystalline 2C-B.sulfate. The XRPD signals observed in this diffractogram are characterized in Table 26.

FIG. 16 depicts an XRPD diffractogram of crystalline 2C-B.phosphate (Form 1 and Form 2). The XRPD signals observed in this diffractogram are characterized in Table 27.

FIG. 17 provides an XRPD diffractogram of crystalline 2C-B.xinafoate. The XRPD signals observed in this diffractogram are characterized in Table 28.

FIG. 18 depicts an XRPD diffractogram of crystalline 2C-B.lactate (Form 1). The XRPD signals observed in this diffractogram are characterized in Table 29.

FIG. 19 depicts an XRPD diffractogram of crystalline 2C-B.malate+peaks. The XRPD signals observed in this diffractogram are characterized in Table 30. The XRPD signals include the signals corresponding to 2C-B malate and other signals.

FIG. 20 depicts an XRPD diffractogram of crystalline 2C-B.maleate (Form 1). The XRPD signals observed in this diffractogram are characterized in Table 31.

FIG. 21 depicts an XRPD diffractogram of crystalline 2C-B.malonate. The XRPD signals observed in this diffractogram are characterized in Table 32.

FIG. 22 depicts an XRPD diffractogram of crystalline 2C-B.mesylate. The XRPD signals observed in this diffractogram are characterized in Table 33.

FIG. 23 depicts an XRPD diffractogram of crystalline 2C-B.mucate. The XRPD signals observed in this diffractogram are characterized in Table 34.

FIG. 24 depicts an XRPD diffractogram of crystalline 2C-B.succinate Forms 1 and 2 plus free succinic acid. The XRPD signals observed in this diffractogram are characterized in Table 35.

FIG. 25 depicts an XRPD diffractogram of crystalline 2C-B.tartrate (Form 1). The XRPD signals observed in this diffractogram are characterized in Table 36.

FIG. 26 depicts an XRPD diffractogram of crystalline 2C-B.tosylate (Form 1). The XRPD signals observed in this diffractogram are characterized in Table 37.

FIG. 27 depicts a 1H NMR spectrum of 2C-B free base.

FIG. 28 depicts the structural formula of “4-bromo-2,5-dimethoxyphenethylamine”.

FIG. 29 depicts an XRPD diffractogram of crystalline 2C-B.besylate (Form 2). The XRPD signals observed in this diffractogram are characterized in Table 38.

FIG. 30 depicts an XRPD diffractogram of crystalline 2C-B.esylate (Form 2). The XRPD signals observed in this diffractogram are characterized in Table 39.

FIG. 31 depicts an XRPD diffractogram of crystalline 2C-B.phosphate (Form 2). The XRPD signals observed in this diffractogram are characterized in Table 40.

FIG. 32 depicts an XRPD diffractogram of crystalline 2C-B.lactate (Form 2). The XRPD signals observed in this diffractogram are characterized in Table 41.

FIG. 33 depicts an XRPD diffractogram of crystalline 2C-B.maleate (Form 2). The XRPD signals observed in this diffractogram are characterized in Table 42.

FIG. 34 depicts an XRPD diffractogram of crystalline 2C-B succinate (Form 1). The XRPD signals observed in this diffractogram are characterized in Table 43.

FIG. 35 depicts an XRPD diffractogram of a sample comprising 2C-B tosylate (Forms 1 and 2) plus free toluene sulfonic acid. The XRPD signals observed in this diffractogram are characterized in Table 44.

FIG. 36 depicts the structure formula of mescaline HCl.

FIG. 37 depicts an XRPD diffractogram of a sample comprising 2C-B.aspartate. The XRPD signals observed in this diffractogram are characterized in Table 45. The XRPD signals include the signals corresponding to 2C-B aspartate and other signals.

FIG. 37B depicts an XRPD diffractogram of a sample comprising 2C-B.aspartate. The XRPD signals include the signals corresponding to 2C-B aspartate and Free Base (Form 1) signals.

FIG. 38 depicts an XRPD diffractogram of a sample comprising 2C-B.glutamate (PO). The XRPD signals observed in this diffractogram are characterized in Table 46.

FIG. 39 depicts an XRPD diffractogram of a sample comprising crystalline 2C-B citrate (Form 2). The XRPD signals observed in this diffractogram are characterized in Table 47.

FIG. 40 depicts XRPD patterns of lots of 2C-B HCl (Form A).

FIG. 41 depicts an XRPD diffractogram of 2C-B HCl (Form B). The XRPD signals observed in this diffractogram are characterized in Table 57.

FIG. 42 depicts an XRPD overlay of 2C-B HCl polymorphs.

FIG. 43 depicts an XRPD diffractogram of 2C-B HCl Form A. The XRPD signals observed in this diffractogram are characterized in Table 58.

FIG. 44 depicts an XRPD pattern of Form A with preferred orientation.

FIG. 45 depicts a proton NMR spectrum of lot of 2C-B.HCl (Form A).

FIG. 46 depicts a proton NMR spectrum of 2C-B.HCl (Form B).

FIG. 47 depicts a proton NMR spectrum of 2C-B.HCl (Form C).

FIG. 48 depicts a proton NMR spectrum of 2C-B (Free Base).

FIG. 49 depicts a proton NMR spectrum of 2C-B (Free Base), 3 gram scale.

FIG. 50 depicts the TGA and DSC profiles of lot of 2C-B.HCl (Form A).

FIG. 51 depicts the TGA and DSC profiles of 2C-B.HCl (Form B).

FIG. 52 depicts the TGA and DSC profiles of 2C-B.HCl (Form C).

FIG. 53 depicts the DVS profile of 2C-B.HCl (Form A).

FIG. 54 depicts the IR spectrum of the 2C-B.HCl (Form A).

FIG. 55 depicts an XRPD diffractogram of 2C-B B.esylate (Form 1). The XRPD signals observed in this diffractogram are characterized in Table 61.

FIG. 56 depicts an XRPD diffractogram of 2C-B Gentisate. The XRPD signals observed in this diffractogram are characterized in Table 62.

FIG. 57 depicts an XRPD diffractogram of 2C-B Gentisate (PO). The XRPD signals observed in this diffractogram are characterized in Table 63.

FIG. 58 depicts an XRPD diffractogram of 2C-B Glutamate. The XRPD signals observed in this diffractogram are characterized in Table 64.

FIG. 59 depicts an XRPD diffractogram of 2C-B Glycolate. The XRPD signals observed in this diffractogram are characterized in Table 65.

FIG. 60 depicts an XRPD diffractogram of 2C-B Malate. The XRPD signals observed in this diffractogram are characterized in Table 66.

FIG. 61 depicts an XRPD diffractogram of 2C-B Mesylate. The XRPD signals observed in this diffractogram are characterized in Table 67.

FIG. 62 depicts an XRPD diffractogram of 2C-B Sulfate. The XRPD signals observed in this diffractogram are characterized in Table 68.

FIG. 63 depicts an XRPD diffractogram of 2C-B Xinafoate. The XRPD signals observed in this diffractogram are characterized in Table 69.

FIG. 64 depicts an XRPD diffractogram of 2C-B Tartrate (Form 1). The XRPD signals observed in this diffractogram are characterized in Table 70.

FIG. 65 depicts an XRPD diffractogram of 2C-B Tartrate (Form 2). The XRPD signals observed in this diffractogram are characterized in Table 71.

FIG. 66 depicts XRPD patterns of 2C-B Tartrate polymorphs.

FIG. 67 depicts an XRPD patterns of unique materials made with 2C-B and benzenesulfonic acid, citric acid, ethanesulfonic acid, and fumaric acid.

FIG. 68 depicts XRPD patterns of unique materials made with 2C-B and gentisic acid, glycolic acid, L-glutamic acid, D,L-lactic acid, and L-malic acid.

FIG. 69 depicts XRPD patterns of unique materials made with 2C-B and maleic acid, methanesulfonic acid, mucic (galactaric) acid, and phosphoric acid.

FIG. 70 depicts XRPD patterns of unique materials made with 2C-B and sulfuric acid, succinic acid, and L-tartaric acid.

FIG. 71 depicts XRPD patterns of unique materials made with 2C-B and p-toluenesulfonic acid and xinafoic (1-hydroxy-2-naphthoic) acid.

FIG. 72 depicts an XRPD patterns of solids produced from experiments with 2C-B and malonic acid, L-aspartic acid, and D-gluconic acid

FIG. 73 depicts a proton NMR spectrum of 2C-B B.esylate (Form 1).

FIG. 74 depicts a proton NMR spectrum of 2C-B B.esylate (Form 2).

FIG. 75 depicts a proton NMR spectrum of 2C-B Citrate (Form 1).

FIG. 76 depicts a proton NMR spectrum of 2C-B Esylate (Form 1).

FIG. 77 depicts a proton NMR spectrum of 2C-B Esylate (Form 2).

FIG. 78 depicts a proton NMR spectrum of 2C-B Fumarate (Form 1).

FIG. 79 depicts a proton NMR spectrum of 2C-B Gentisate.

FIG. 80 depicts a proton NMR spectrum of 2C-B Glutamate.

FIG. 81 depicts a proton NMR spectrum of 2C-B Glycolate.

FIG. 82 depicts a proton NMR spectrum of 2C-B Lactate (Form 1).

FIG. 83 depicts a proton NMR spectrum of 2C-B Lactate (Form 2).

FIG. 84 depicts a proton NMR spectrum of 2C-B Malate.

FIG. 85 depicts a proton NMR spectrum of 2C-B Maleate (Form 1).

FIG. 86 depicts a proton NMR spectrum of 2C-B Maleate (Form 2).

FIG. 87 depicts a proton NMR spectrum of 2C-B Mesylate.

FIG. 88 depicts a proton NMR spectrum of 2C-B Mucate.

FIG. 89 depicts a proton NMR spectrum of 2C-B Phosphate (Form 1).

FIG. 90 depicts a proton NMR spectrum of 2C-B Phosphate (Form 2).

FIG. 91 depicts a proton NMR spectrum of 2C-B Succinate (Form 1).

FIG. 92 depicts a proton NMR spectrum of 2C-B Sulfate.

FIG. 93 depicts a proton NMR spectrum of 2C-B Tartrate (Form 1).

FIG. 94 depicts a proton NMR spectrum of 2C-B Tosylate (Form 1).

FIG. 95 depicts a proton NMR spectrum of 2C-B Xinafoate.

FIG. 96 depicts a proton NMR spectrum of 2C-B gluconic acid salt attempt.

FIG. 97 depicts a proton NMR spectrum of 2C-B malonic acid salt attempt.

FIG. 98 depicts a proton NMR spectrum of 2C-B B.esylate (Form 1).

FIG. 99 depicts a proton NMR spectrum of 2C-B Citrate (Form 2).

FIG. 100 depicts a proton NMR spectrum of 2C-B Gentisate.

FIG. 101 depicts a proton NMR spectrum of 2C-B Glycolate.

FIG. 102 depicts a proton NMR spectrum of 2C-B Malate.

FIG. 103 depicts a proton NMR spectrum of 2C-B Mesylate.

FIG. 104 depicts a proton NMR spectrum of 2C-B Sulfate.

FIG. 105 depicts a proton NMR spectrum of 2C-B Tartrate (Form 1).

FIG. 106 depicts a proton NMR spectrum of 2C-B Tartrate (Form 2).

FIG. 107 depicts the TGA and DSC profiles of 2C-B B.esylate (Form 1).

FIG. 108 depicts the TGA and DSC profiles of 2C-B B.esylate (Form 2).

FIG. 109 depicts the TGA and DSC profiles of 2C-B Citrate (Form 1).

FIG. 110 depicts the TGA and DSC profiles of 2C-B Esylate (Form 1).

FIG. 111 depicts the TGA and DSC profiles of 2C-B Esylate (Form 2).

FIG. 112 depicts the TGA and DSC profiles of 2C-B Fumarate (Form 1).

FIG. 113 depicts the TGA and DSC profiles of 2C-B Gentisate.

FIG. 114 depicts the TGA and DSC profiles of 2C-B Glutamate.

FIG. 115 depicts the TGA and DSC profiles of 2C-B Glycolate.

FIG. 116 depicts the TGA and DSC profiles of 2C-B Malate.

FIG. 117 depicts the TGA and DSC profiles of 2C-B Maleate (Form 1).

FIG. 118 depicts the TGA and DSC profiles of 2C-B Mesylate.

FIG. 119 depicts the TGA and DSC profiles of 2C-B Phosphate (Form 1).

FIG. 120 depicts the TGA and DSC profiles of 2C-B Succinate (Form 1).

FIG. 121 depicts the TGA and DSC profiles of 2C-B Sulfate.

FIG. 122 depicts the TGA and DSC profiles of 2C-B Tartrate (Form 1).

FIG. 123 depicts the TGA and DSC profiles of 2C-B Tosylate (Form 1).

FIG. 124 depicts the TGA and DSC profiles of 2C-B Xinafoate.

FIG. 125 depicts the TGA and DSC profiles of 2C-B B.esylate (Form 1).

FIG. 126 depicts the TGA and DSC profiles of 2C-B Citrate (Form 2).

FIG. 127 depicts the TGA and DSC profiles of 2C-B Gentisate.

FIG. 128 depicts the TGA and DSC profiles of 2C-B Glycolate.

FIG. 129 depicts the TGA and DSC profiles of 2C-B Malate.

FIG. 130 depicts the TGA and DSC profiles of 2C-B Mesylate.

FIG. 131 depicts the TGA and DSC profiles of 2C-B Sulfate.

FIG. 132 depicts the TGA and DSC profiles of 2C-B Tartrate (Form 1).

FIG. 133 depicts the TGA and DSC profiles of 2C-B Tartrate (Form 2).

FIG. 134 depicts the TGA and DSC profiles of non-crystalline 2C-B Tartrate.

FIG. 135 depicts the thermal profiles of non-crystalline 2C-B Tartrate.

FIG. 135B depicts the modulated DSC of non-crystalline 2C-B tartrate (with drying cycle)

FIG. 136 depicts the thermal profiles of non-crystalline 2C-B Tartrate.

FIG. 137 depicts the DVS profile of 2C-B B.esylate (Form 1).

FIG. 138 depicts the DVS profile of 2C-B Citrate (Form 2).

FIG. 139 depicts the DVS profile of 2C-B Gentisate.

FIG. 140 depicts the DVS profile of 2C-B Glycolate.

FIG. 141 depicts the DVS profile of 2C-B Malate.

FIG. 142 depicts the DVS profile of 2C-B Mesylate.

FIG. 143 depicts the DVS profile of 2C-B Sulfate.

FIG. 144 depicts the DVS profile of 2C-B Tartrate (Form 1).

FIG. 145A depicts the indexing solution for 2C-B Malate.

FIG. 145B depicts XRPD comparison of samples of 2C-B Malate with additional peaks (red and blue patterns) against the indexed pattern of 2C-B Malate (bottom, black pattern)

FIG. 146 depicts XRPD patterns of 2C-B Tartrate polymorphs.

FIG. 146B depicts the XRPD patterns of 2C-B tartrate polymorphs.

FIG. 146C depicts the XRPD pattern of non-crystalline 2C-B tartrate post-heating at 110° C.

FIG. 146D depicts the DVS profile of non-crystalline 2C-B tartrate.

FIG. 147 depicts the asymmetric unit from the crystal structure. Carbon atoms are gray, nitrogen atoms are blue, oxygen atoms are red, bromine atom is orange, and hydrogen atoms are white.

FIG. 148 depicts a packing diagram looking down the a axis. Carbon atoms are gray, nitrogen atoms are blue, oxygen atoms are red, bromine atoms are orange, and hydrogen atoms are white.

FIG. 149 depicts a packing diagram looking down the b axis. Carbon atoms are gray, nitrogen atoms are blue, oxygen atoms are red, bromine atoms are orange, and hydrogen atoms are white.

FIG. 150 depicts a packing diagram looking down the c axis. Carbon atoms are gray, nitrogen atoms are blue, oxygen atoms are red, bromine atoms are orange, and hydrogen atoms are white.

FIG. 151 is an overlay plot of the XRPD pattern of 2C-B tartrate (Form 1) calculated from single-crystal data (top red trace) with a pattern obtained from the bulk material (bottom blue trace).

FIGS. 152A-E are XRPD pattern of 2C-B tartrate (Form 1) calculated from single-crystal data.

FIG. 153 depicts the XRPD pattern of non-crystalline 2C-B tartrate.

FIG. 154 depicts the ¹H NMR spectrum of non-crystalline 2C-B tartrate.

FIG. 155 depicts the TG of non-crystalline 2C-B tartrate.

FIG. 156 depicts the DSC of non-crystalline 2C-B tartrate.

FIG. 157 depicts the modulated DSC of non-crystalline 2C-B tartrate.

FIG. 158 depicts the XRPD pattern of post-DVS sample.

FIGS. 159A-D depict the XRPD pattern of Mescaline HCl.

FIG. 160 illustrates the frequency of rearing after 2C-B compared to vehicle and chlordiazepoxide control on the elevated zero maze. **p<0.01 significantly different from relevant vehicle control by Newman-Keuls hoc test following significant one-way ANOVA.

FIG. 161 illustrates the amount of TNF-alpha release in whole blood without LPS after 2C-B, saline, or dexamethasone. This data was analyzed by 1-way ANOVA.

FIG. 162 illustrates the amount of TNF-alpha release in whole blood in response to LPS 1 μg/ml after 2C-B compared to saline and dexamethasone control. This data was analyzed by 1-way ANOVA.

FIG. 163 illustrates the amount of TNF-alpha release in whole blood in response to LPS 3 μg/ml after 2C-B compared to saline and dexamethasone control. This data was analyzed by 1-way ANOVA.

FIG. 164 illustrates the amount of TNF-alpha release in whole blood in response to LPS 1 μg/ml and 3 μg/ml after 2C-B compared to saline and dexamethasone control. This data was analyzed by 2-way ANOVA. Each treatment group was compared to the vehicle group and a p-value for treatment corrected for multiplicity by Dunnett's test was determined.

FIG. 165 illustrates the EXD data of 2C-B sulfate salt.

### DETAILED DESCRIPTION

### I. Definitions

The following explanations of terms and methods are provided to better describe the present disclosure and to guide those of ordinary skill in the art in the practice of the present disclosure. The singular forms “a,” “an,” and “the” refer to one or more than one, unless the context clearly dictates otherwise. The term “or” refers to a single element of stated alternative elements or a combination of two or more elements, unless the context clearly indicates otherwise. As used herein, “comprises” means “includes.” Thus, “comprising A or B,” means “including A, B, or A and B,” without excluding additional elements. All references, including patents and patent applications cited herein, are incorporated by reference in their entirety, unless otherwise specified.

Unless otherwise indicated, all numbers expressing quantities of components, molecular weights, percentages, temperatures, times, and so forth, as used in the specification or claims, are to be understood as being modified by the term “about.” Accordingly, unless otherwise indicated, implicitly or explicitly, the numerical parameters set forth are approximations that may depend on the desired properties sought and/or limits of detection under standard test conditions/methods. When directly and explicitly distinguishing embodiments from discussed prior art, the embodiment numbers are not approximates unless the word “about” is expressly recited. When used in the context of XRPD signal values, the term “about” can indicate a peak value ±0.20, ±0.15, ±0.10, ±0.05, or ±0.01 °2θ. In some embodiments, when used in the context of XRPD signal values “about” can indicate a peak value at substantially exactly the disclosed peak value.

The terms “XRPD peak”, “XRPD signal” and “XRPD peak/signal” are used interchangeably.

In some embodiments, salts and polymorphic forms thereof described herein are characterized by two or more, or three XRPD signals selected from a group of XRPD signals. In some embodiments, the salts and polymorphic forms thereof described herein are characterized by one, two, three, four, five, six, seven, eight, nine, or ten of the XRPD signals described in the corresponding XRPD table in the Examples. In some embodiments, the salts and polymorphic forms thereof described herein are characterized by one, two, three, four, five, six, seven, eight, nine, or ten of the XRPD signals described in the corresponding XRPD table located in the Examples starting from the most intense peaks. In some embodiments, the term “two or more” encompasses an embodiment where all of the XRPD peaks listed in the embodiment are selected.

Unless explained otherwise, all technical and scientific terms used herein have the same meaning as commonly understood by one of ordinary skill in the art to which this disclosure pertains. Although methods and materials similar or equivalent to those described herein can be used in the practice or testing of the present disclosure, suitable methods and materials are described below. The materials, methods, and examples are illustrative only and not intended to be limiting.

“Administering” refers to any suitable mode of administration, including, oral administration, administration as a suppository, topical contact, parenteral, intravenous, intraperitoneal, intramuscular, intralesional, intranasal or subcutaneous administration, intrathecal administration, or the implantation of a slow-release device e.g., a mini-osmotic pump, to the subject.

“4-bromo-2,5-dimethoxyphenethylamine” refers to the compound of structural formula:

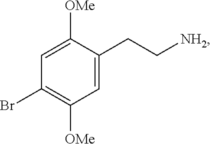 [SMILES: COc1cc(CCN)c(OC)cc1Br]

which also may be referred to herein as compound 1, 2C-B, or 2C-B free base.

“4-bromo-2,5-dimethoxyphenethylamine hydrochloride” refers to the hydrochloride salt of the compound

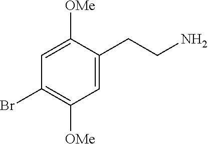 [SMILES: COc1cc(CCN)c(OC)cc1Br]

also represented as

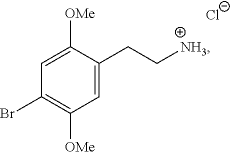

which also may be referred to herein as 4-bromo-2,5-dimethoxyphenethylamine.HCl; 2C-B hydrochloride or 2C-B.HCl.

“Subject” refers to an animal, such as a mammal, including, but not limited to, primates (e.g., humans), cows, sheep, goats, horses, dogs, cats, rabbits, rats, mice and the like. In certain embodiments, the subject is a human subject.

“Therapeutically effective amount” or “therapeutically sufficient amount” or “effective or sufficient amount” refers to a dose that produces therapeutic effects for which it is administered. The exact dose will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins). In sensitized cells, the therapeutically effective dose can often be lower than the conventional therapeutically effective dose for non-sensitized cells.

“Neuronal plasticity” refers to the ability of the brain to change its structure and/or function continuously throughout a subject's life. Examples of the changes to the brain include, but are not limited to, the ability to adapt or respond to internal and/or external stimuli, such as due to an injury, and the ability to produce new neurites, dendritic spines, and synapses.

“Brain disorder” refers to a neurological disorder which affects the brain's structure and function. Brain disorders can include, but are not limited to, Alzheimer's, Parkinson's disease, psychological disorder, depression, treatment resistant depression, addiction, anxiety, post-traumatic stress disorder, suicidal ideation, major depressive disorder, bipolar disorder, schizophrenia, stroke, traumatic brain injury, and substance use disorder.

“Combination therapy” refers to a method of treating a disease or disorder, wherein two or more different pharmaceutical agents are administered in overlapping regimens so that the subject is simultaneously exposed to both agents. For example, the compounds of the invention can be used in combination with other pharmaceutically active compounds. The compounds of the invention can be administered simultaneously (as a single preparation or separate preparation) or sequentially to the other drug therapy. In general, a combination therapy envisions administration of two or more drugs during a single cycle or course of therapy.

“Neurotrophic factors” refers to a family of soluble peptides or proteins which support the survival, growth, and differentiation of developing and mature neurons.

“Modulate” or “modulating” or “modulation” refers to an increase or decrease in the amount, quality, or effect of a particular activity, function or molecule. By way of illustration and not limitation, agonists, partial agonists, antagonists, and allosteric modulators (e.g., a positive allosteric modulator) of a G protein-coupled receptor (e.g., 5HT_{2A}) are modulators of the receptor.

“Agonism” refers to the activation of a receptor or enzyme by a modulator, or agonist, to produce a biological response.

“Agonist” refers to a modulator that binds to a receptor or enzyme and activates the receptor to produce a biological response. By way of example only, “5HT_{2A} agonist” can be used to refer to a compound that exhibits an EC₅₀ with respect to 5HT_{2A} activity of no more than about 100 mM. In some embodiments, the term “agonist” includes full agonists or partial agonists. “Full agonist” refers to a modulator that binds to and activates a receptor with the maximum response that an agonist can elicit at the receptor. “Partial agonist” refers to a modulator that binds to and activates a given receptor, but has partial efficacy, that is, less than the maximal response, at the receptor relative to a full agonist.

“Positive allosteric modulator” refers to a modulator that binds to a site distinct from the orthosteric binding site and enhances or amplifies the effect of an agonist.

“Antagonism” refers to the inactivation of a receptor or enzyme by a modulator, or antagonist. Antagonism of a receptor, for example, is when a molecule binds to the receptor and does not allow activity to occur.

“Antagonist” or “neutral antagonist” refers to a modulator that binds to a receptor or enzyme and blocks a biological response. An antagonist has no activity in the absence of an agonist or inverse agonist but can block the activity of either, causing no change in the biological response.

“Composition” refers to a product comprising the specified ingredients in the specified amounts, as well as any product, which results, directly or indirectly, from combination of the specified ingredients in the specified amounts. By “pharmaceutically acceptable” it is meant the carrier, diluent or excipient must be compatible with the other ingredients of the formulation.

“Pharmaceutically acceptable excipient” refers to a substance that aids the administration of an active agent to and absorption by a subject. Pharmaceutical excipients useful in the present invention include, but are not limited to, binders, fillers, disintegrants, lubricants, coatings, sweeteners, flavors and colors. One of skill in the art will recognize that other pharmaceutical excipients are useful in the present invention.

The terms “powder X-ray diffraction pattern”, “PXRD pattern”, “X-ray powder diffraction pattern”, and “XRPD pattern” are used interchangeably and refer to the experimentally observed diffractogram or parameters derived therefrom. Powder X-ray diffraction patterns are typically characterized by peak position (abscissa) and peak intensities (ordinate). The term “peak intensities” refers to relative signal intensities within a given X-ray diffraction pattern. Factors which can affect the relative peak intensities are sample thickness and preferred orientation (PO) (i.e., the crystalline particles are not distributed randomly). The term “peak positions” as used herein refers to X-ray reflection positions as measured and observed in powder X-ray diffraction experiments. Peak positions are directly related to the dimensions of the unit cell. The peaks, identified by their respective peak positions, are extracted from the diffraction patterns for the various polymorphic forms of the salts of compound 1.

The term “2 theta value”, “2θ” or “2 θ” refers to the peak position in degrees based on the experimental setup of the X-ray diffraction experiment and is a common abscissa unit in diffraction patterns. In general, the experimental setup requires that if a reflection is diffracted when the incoming beam forms an angle theta (θ) with a certain lattice plane, the reflected beam is recorded at an angle 2 theta (2 θ). It should be understood that reference herein to specific 2θ values for a specific polymorphic form is intended to mean the 2θ values (in degrees) as measured using the X-ray diffraction experimental conditions as described herein.

“Preferred orientation effects” refer to variable peak intensities or relative intensity differences between different PXRD measurements of the same samples that can be due to the orientation of the particles. Without wishing to be bound by theory, in PXRD it can be desirable to have a sample in which particles are oriented randomly (e.g., a powder). However, it can be difficult or in some cases impossible to achieve truly random particle orientations in practice. As particle size increases, the randomness of particle orientation can decrease, leading to increased challenges with achieving a preferred orientation. Without wishing to be bound by theory, a smaller particle size can reduce technical challenges associated with preferred orientation and allow for more accurate representation of peaks. However, one of skill in the art will understand how to reduce or mitigate preferred orientation effects and will recognize preferred orientation effects that can exist even between two different measurements of the same sample. For instance, in some embodiments, differences in resolution or relative peak intensities can be attributed to preferred orientation effects.

As used herein, the term “substantially pure” with reference to a particular salt or solid form (or to a mixture of two or more salts) of a compound indicates the salt or solid form (or a mixture) includes less than 10%, less than 5%, less than 3%, less than 1%, less than 0.5%, less than 0.2%, or less than 0.1% by weight of impurities, including other salt or solid forms of the compound. Such purity may be determined, for example, by powder X-ray diffraction.

As used herein, the term “polymorph” or “salt form” refers to different crystalline forms of the same compound and other solid state molecular forms including pseudo-polymorphs, such as hydrates (e.g., bound water present in the crystalline structure) and solvates (e.g., bound solvents other than water) of the same compound. Different crystalline polymorphs have different crystal structures due to a different packing of the molecules in the lattice. This results in a different crystal symmetry and/or unit cell parameters which directly influences its physical properties such as the X-ray diffraction characteristics of crystals or powders. A different polymorph, for example, will in general diffract at a different set of angles and will give different values for the intensities. Therefore, X-ray powder diffraction can be used to identify different polymorphs, or a solid form that comprises more than one polymorph, in a reproducible and reliable way (S. Byrn et al, Pharmaceutical Solids: A Strategic Approach to Regulatory Considerations, Pharmaceutical research, Vol. 12, No. 7, p. 945-954, 1995; J. K. Haleblian and W. McCrone, Pharmacetical Applications of Polymorphism, Journal of Pharmaceutical Sciences, Vol. 58, No. 8, p. 91 1-929, 1969).

Crystalline polymorphic forms are of interest to the pharmaceutical industry and especially to those involved in the development of suitable dosage forms. If the polymorphic form is not held constant during clinical or stability studies, the exact dosage form used or studied may not be comparable from one lot to another. It is also desirable to have processes for producing a compound with the selected polymorphic form in high purity when the compound is used in clinical studies or commercial products since impurities present may produce undesired toxicological effects. Certain polymorphic forms may exhibit enhanced thermodynamic stability or may be more readily manufactured in high purity in large quantities, and thus are more suitable for inclusion in pharmaceutical formulations. Certain polymorphs may display other advantageous physical properties such as lack of hygroscopic tendencies, improved solubility, and enhanced rates of dissolution due to different lattice energies.

The term “amorphous” refers to any solid substance which (i) lacks order in three dimensions, or (ii) exhibits order in less than three dimensions, order only over short distances (e.g., less than 10 A), or both. Thus, amorphous substances include partially crystalline materials and crystalline mesophases with, e.g., one- or two-dimensional translational order (liquid crystals), orientational disorder (orientationally disordered crystals), or conformational disorder (conformationally disordered crystals). Amorphous solids may be characterized by known techniques, including powder X-ray diffraction (PXRD) crystallography, solid state nuclear magnet resonance (ssNMR) spectroscopy, differential scanning calorimetry (DSC), or some combination of these techniques. Amorphous solids give diffuse PXRD patterns, typically comprised of one or two broad peaks (i.e., peaks having base widths of about 5 °2Θ or greater).

The term “crystalline” refers to any solid substance exhibiting three-dimensional order, which in contrast to an amorphous solid substance, gives a distinctive PXRD pattern with sharply defined peaks.

The term “ambient temperature” refers to a temperature condition typically encountered in a laboratory setting. This includes the approximate temperature range of about 20 to about 30° C.

The term “detectable amount” refers to an amount or amount per unit volume that can be detected using conventional techniques, such as X-ray powder diffraction, differential scanning calorimetry, HPLC, Fourier Transform Infrared Spectroscopy (FT-IR), Raman spectroscopy, and the like.

The term “solvate” describes a molecular complex comprising the drug substance and a stoichiometric or non-stoichiometric amount of one or more solvent molecules (e.g., ethanol). When the solvent is tightly bound to the drug the resulting complex will have a well-defined stoichiometry that is independent of humidity. When, however, the solvent is weakly bound, as in channel solvates and hygroscopic compounds, the solvent content will be dependent on humidity and drying conditions. In such cases, the complex may be non-stoichiometric.

The term “hydrate” describes a solvate comprising the drug substance and a stoichiometric or non-stoichiometric amount of water.

The term “relative humidity” refers to the ratio of the amount of water vapor in air at a given temperature to the maximum amount of water vapor that can be held at that temperature and pressure, expressed as a percentage.

The term “relative intensity” refers to an intensity value derived from a sample X-ray diffraction pattern. The complete ordinate range scale for a diffraction pattern is assigned a value of 100. A peak having intensity falling between about 50% to about 100% on this scale intensity is termed very strong (vs); a peak having intensity falling between about 50% to about 25% is termed strong (s). Additional weaker peaks are present in typical diffraction patterns and are also characteristic of a given polymorph, wherein the additional peaks are termed medium (m), weak (w) and very weak (vw).

The term “slurry” refers to a solid substance suspended in a liquid medium, typically water or an organic solvent.

The term “under vacuum” refers to typical pressures obtainable by a laboratory oil or oil-free diaphragm vacuum pump.

The term “treating”, as used herein, unless otherwise indicated, means reversing, alleviating, or inhibiting the progress of the disorder or condition to which such term applies, or one or more symptoms of such disorder or condition. The term “treatment”, as used herein, unless otherwise indicated, refers to the act of “treating” as defined immediately above. For example, the terms “treat”, “treating” and “treatment” can refer to a method of alleviating or abrogating a particular disorder and/or one or more of its attendant symptoms.

The term “pharmaceutical composition” refers to a composition comprising one or more of the salt or solid forms of compound 1 described herein, and other chemical components, such as physiologically/pharmaceutically acceptable carriers, diluents, vehicles and/or excipients. The purpose of a pharmaceutical composition is to facilitate administration of a compound to an organism, such as a human or other mammals.

The term “pharmaceutically acceptable” “carrier”, “diluent”, “vehicle”, or “excipient” refers to a material (or materials) that may be included with a particular pharmaceutical agent to form a pharmaceutical composition, and may be solid or liquid. Exemplary solid carriers are lactose, sucrose, talc, gelatin, agar, pectin, acacia, magnesium stearate, stearic acid and the like. Exemplary liquid carriers are syrup, peanut oil, olive oil, water and the like. Similarly, the carrier or diluent may include time-delay or time-release material known in the art, such as glyceryl monostearate or glyceryl distearate alone or with a wax, ethylcellulose, hydroxypropyl methylcellulose, methylmethacrylate and the like.

The term “compound of the present disclosure”, “compounds of the present disclosure”, “presently disclosed compound”, “presently disclosed compounds”, “compound disclosed herein”, or “compounds disclosed herein” means the salt and solid form(s) of compound 1 or the free base of compound 1.

### II. Compounds

Disclosed herein are solid forms of 4-bromo-2,5-dimethoxyphenethylamine hydrochloride that are useful to treat various disorders, such as brain disorders. Also disclosed are methods for making the solid forms of the compounds and method of administering the solid forms of the compounds.

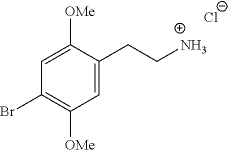 [SMILES: COc1cc(CC[NH3+])c(OC)cc1Br ; [Cl-]]

### 4-bromo-2,5-dimethoxyphenethylamine hydrochloride (2C-B.HCl)

With reference to the formula for 2C-B.HCl, the middle dot, “.”, represents that the compound is the acid addition salt of 4-bromo-2,5-dimethoxyphenethylamine. In some embodiments, the solid form of the compound is a crystalline form of the compound. In some embodiments, the solid form of the compound is a polymorph of the compound, such as a novel polymorph that is not previously known in the art.

In some embodiments, disclosed salts and solid forms do not include 4-bromo-2,5-dimethoxyphenethylamine hydrochloride (2C-B.HCl).

Also disclosed herein are salts and solid forms of 4-bromo-2,5-dimethoxyphenethylamine (2C-B) that are useful to treat various disorders, such as brain disorders. Also disclosed are methods for making the salts and solid forms of 4-bromo-2,5-dimethoxyphenethylamine and method of administering the solid forms of 4-bromo-2,5-dimethoxyphenethylamine.

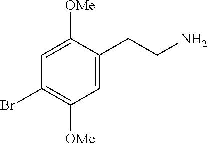 [SMILES: COc1cc(CCN)c(OC)cc1Br]

### 4-bromo-2,5-dimethoxyphenethylamine

In some embodiments, the solid form of the compound is a crystalline form of 4-bromo-2,5-dimethoxyphenethylamine. In some embodiments, the solid form of 4-bromo-2,5-dimethoxyphenethylamine is a polymorph of a 4-bromo-2,5-dimethoxyphenethylamine, such as a polymorph of the free base compound or a polymorph of a salt form. In some embodiments, the solid form of the compound is a salt of the compound. In some embodiments, the solid form of the compound is a crystalline salt form of the compound, such as an acid addition salt form.

In yet other embodiments, the salt or solid form of 2C-B is at least about 95% pure as measured by High-performance liquid chromatography (HPLC) or ultraviolet (UV) chromatography.

In yet other embodiments, the salt or solid form of 2C-B is at least about 96% pure as measured by HPLC or UV chromatography.

In yet other embodiments, the salt or solid form of 2C-B is at least about 97% pure as measured by HPLC or UV chromatography.

In yet other embodiments, the salt or solid form of 2C-B is at least about 98% pure as measured by HPLC or UV chromatography.

In yet other embodiments, the salt or solid form of 2C-B is at least about 99% pure as measured by HPLC or UV chromatography.

In yet other embodiments, the salt or solid form of 2C-B is at least about 99.5% pure as measured by HPLC or UV chromatography.

In some embodiments, the 2C-B (free base Forms 1 and 2) is a crystalline polymorph characterized by two or more, or three XRPD signals selected from the group consisting of 24.3 °2θ, 23.6 °2θ, and 17 °2θ (±0.2 °2θ; ±0.1 °2θ; or ±0.0 °2θ; Cu Kα1 radiation).

In some embodiments, the 2C-B (free base Forms 1 and 2) is a crystalline polymorph characterized by two or more, or three XRPD signals selected from the group consisting of 24.3 °2θ, 23.6 °2θ, 17 °2θ, and 25.3 °2θ (±0.2 °2θ; ±0.1 °2θ; or ±0.0 °2θ; Cu Kα1 radiation).

In some embodiments, the 2C-B (free base Forms 1 and 2) is a crystalline polymorph characterized by two or more, or three XRPD signals selected from the group consisting of 24.3 °2θ, 23.6 °2θ, 17 °2θ, 25.3 °2θ, and 5.4 °2θ (±0.2 °2θ; ±0.1 °2θ; or ±0.0 °2θ; Cu Kα1 radiation).

In some embodiments, the 2C-B (free base Forms 1 and 2) is a crystalline polymorph characterized by two or more, or three XRPD signals selected from the group consisting of 24.3 °2θ, 23.6 °2θ, 17 °2θ, 25.3 °2θ, 5.4 °2θ, 21.7 °2θ, 26.6 °2θ, 28.2 °2θ, 9.2 °2θ, and 18.6 °2θ (±0.2 °2θ; ±0.1 °2θ; or ±0.0 °2θ; Cu Kα1 radiation).

In some embodiments, the 2C-B (free base Forms 1 and 2) is a crystalline polymorph characterized by two or more, or three XRPD signals selected from the group consisting of 9.2 °2θ, 17 °2θ, and 18.6 °2θ (±0.2 °2θ; ±0.1 °2θ; or ±0.0 °2θ; Cu Kα1 radiation).

In some embodiments, the 2C-B (free base Forms 1 and 2) is a crystalline polymorph characterized by two or more, or three XRPD signals selected from the group consisting of 9.2 °2θ, 17 °2θ, 18.6 °2θ, 23.6 °2θ, and 24.3 °2θ (±0.2 °2θ; ±0.1 °2θ; or ±0.0 °2θ; Cu Kα1 radiation).

In some embodiments, the 2C-B (free base Forms 1 and 2) is a crystalline polymorph characterized by two, three, four, five, six, seven, eight, nine, or ten XRPD signals as shown in Table 10.

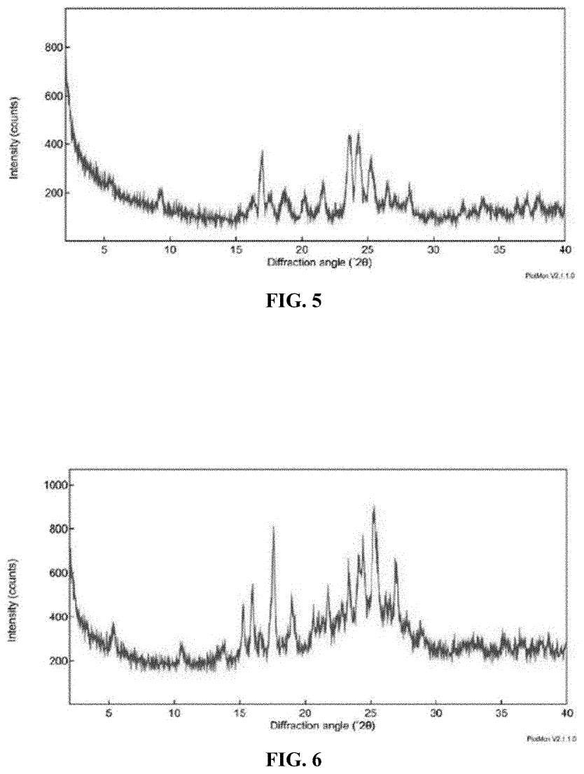

In some embodiments, the 2C-B (free base forms 1 and 2) is a crystalline polymorph characterized by two or more, or three XRPD signals as shown in FIG. 5.

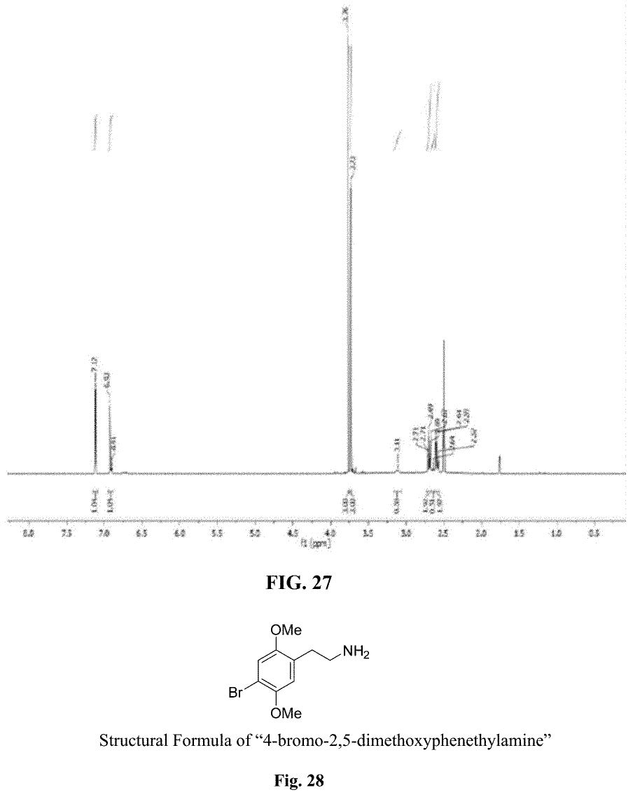

In some embodiments, the 2C-B (free base) has an ¹H NMR spectra as provided in FIG. 27.

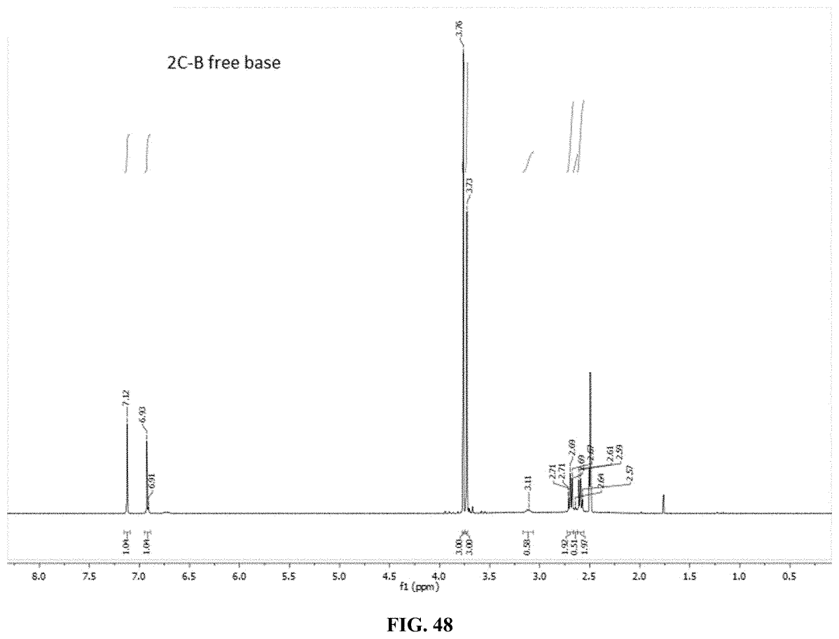

In some embodiments, the 2C-B (free base) has an ¹H NMR spectra as provided in FIG. 48.

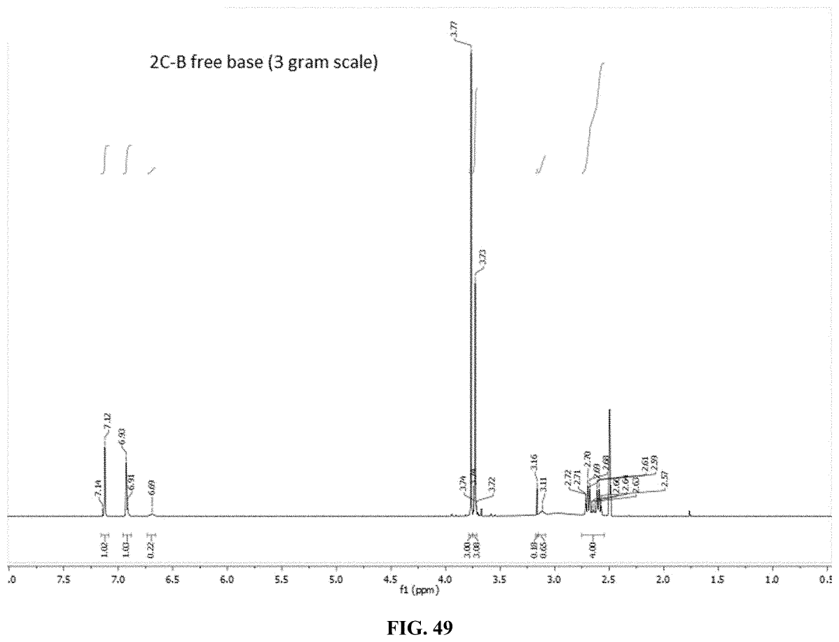

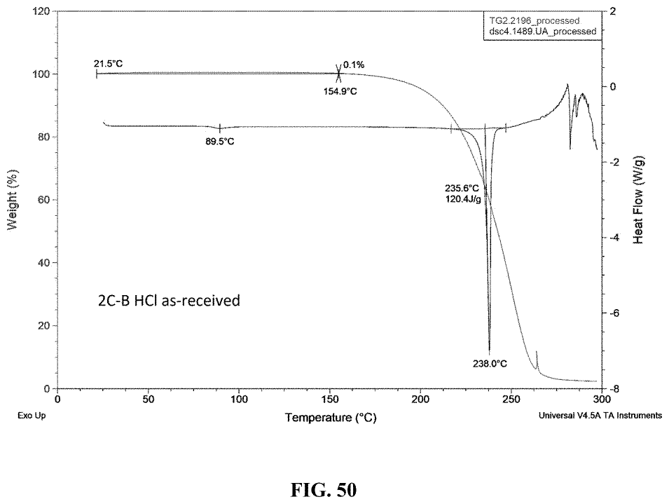

In some embodiments, the 2C-B (free base) has an ¹H NMR spectra as provided in FIG. 49.

In some embodiments, the 2C-B (free base Form 1) is a crystalline polymorph characterized by two or more, or three XRPD signals selected from the group consisting of 25.3 °2θ, 17.6 °2θ, and 24.4 °2θ (±0.2 °2θ; ±0.1 °2θ; or ±0.0 °2θ; Cu Kα1 radiation).

In some embodiments, the 2C-B (free base Form 1) is a crystalline polymorph characterized by two or more, or three XRPD signals selected from the group consisting of 25.3 °2θ, 17.6 °2θ, 24.4 °2θ, and 24.2 °2θ (±0.2 °2θ; ±0.1 °2θ; or ±0.0 °2θ; Cu Kα1 radiation).

In some embodiments, the 2C-B (free base Form 1) is a crystalline polymorph characterized by two or more, or three XRPD signals selected from the group consisting of 25.3 °2θ, 17.6 °2θ, 24.4 °2θ, 24.2 °2θ, and 23.3 °2θ (±0.2 °2θ; ±0.1 °2θ; or ±0.0 °2θ; Cu Kα1 radiation).

In some embodiments, the 2C-B (free base Form 1) is a crystalline polymorph characterized by two or more, or three XRPD signals selected from the group consisting of 25.3 °2θ, 17.6 °2θ, 24.4 °2θ, 24.2 °2θ, 23.3 °2θ, 26.9 °2θ, 16 °2θ, 21.8 °2θ, 26.2 °2θ, and 19 °2θ (±0.2 °2θ; ±0.1 °2θ; or ±0.0 °2θ; Cu Kα1 radiation).

In some embodiments, the 2C-B (free base Form 1) is a crystalline polymorph characterized by two or more, or three XRPD signals selected from the group consisting of 5.3 °2θ, 10.5 °2θ, and 15.3 °2θ (±0.2 °2θ; ±0.1 °2θ; or ±0.0 °2θ; Cu Kα1 radiation).

In some embodiments, the 2C-B (free base Form 1) is a crystalline polymorph characterized by two or more, or three XRPD signals selected from the group consisting of 5.3 °2θ, 10.5 °2θ, and 15.3 °2θ, 16 °2θ, and 17.6 (±0.2 °2θ; ±0.1 °2θ; or ±0.0 °2θ; Cu Kα1 radiation).

In some embodiments, the 2C-B (free base Form 1) is a crystalline polymorph characterized by two, three, four, five, six, seven, eight, nine, or ten XRPD signals as shown in Table 11.

In some embodiments, the 2C-B (free base Form 1) is a crystalline polymorph characterized by two or more, or three XRPD signals as shown in FIG. 6.

### Salts

In some embodiments, the form of 4-bromo-2,5-dimethoxyphenethylamine disclosed herein is a salt form of the compound. In one embodiment, such salts disclosed herein are provided in a solid form, such as wherein the solid form of 4-bromo-2,5-dimethoxyphenethylamine comprises a salt of 4-bromo-2,5-dimethoxyphenethylamine. Suitable salts include a pharmaceutically acceptable salt of 4-bromo-2,5-dimethoxyphenethylamine. In some embodiments, the salt and solid forms of 4-bromo-2,5-dimethoxyphenethylamine is not, and does not comprise, 4-bromo-2,5-dimethoxyphenethylamine hydrochloride.

In some embodiments, the salt of 4-bromo-2,5-dimethoxyphenethylamine may be formed from a suitable pharmaceutically acceptable acid, including, without limitation, inorganic acids such as hydrobromic acid, sulfuric acid, nitric acid, phosphoric acid, and the like, as well as organic acids such as formic acid, acetic acid, trifluoroacetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, benzene sulfonic acid, isethionic acid, methanesulfonic acid, ethanesulfonic acid, p-toluenesulfonic acid, salicylic acid, xinafoic acid and the like.

In other embodiments, the salt of 4-bromo-2,5-dimethoxyphenethylamine may be formed from a suitable pharmaceutically acceptable base, including, without limitation, inorganic bases such as sodium, potassium, lithium, ammonium, calcium, magnesium, iron, zinc, copper, manganese, aluminum salts and the like. Salts derived from pharmaceutically acceptable organic bases include, but are not limited to, salts of primary, secondary, and tertiary amines, substituted amines including naturally occurring substituted amines, cyclic amines and basic ion exchange resins, such as isopropylamine, trimethylamine, diethylamine, triethylamine, tripropylamine, tris(hydroxymethyl)aminomethane (Tris), ethanolamine, 2-dimethylaminoethanol, 2-diethylaminoethanol, dicyclohexylamine, lysine, arginine, histidine, caffeine, procaine, hydrabamine, choline, betaine, ethylenediamine, glucosamine, methylglucamine, theobromine, purines, piperazine, piperidine, N-ethylpiperidine, polyamine resins, and the like. Additional information concerning pharmaceutically acceptable salts can be found in, for example, S. M. Berge, et al., “Pharmaceutical Salts,” J. Pharm. Sci., 1977; 66:1-19 which is incorporated herein by reference.

In some embodiments, the salt may be formed using an acid from Table 1.

TABLE 1

| | |
|---|---|
| naphthalene-1,5-disulfonic acid | citric acid |
| sulfuric acid | d-glucuronic acid |
| ethane-1,2-disulfonic acid | lactobionic acid |
| p-toluenesulfonic acid | D-glucoheptonic acid |
| thiocyanic acid | (−)-L-pyroglutamic acid |
| methanesulfonic acid | L-malic acid |
| dodecylsulfuric acid | hippuric acid |
| naphthalene-2-sulfonic acid | D-gluconic acid |
| benzenesulfonic acid | D,L-lactic acid |
| oxalic acid | oleic acid |
| glycerophosphoric acid | succinic acid |
| ethanesulfonic acid, 2-hydroxy | glutaric acid |
| L-aspartic acid | cinnamic acid |
| maleic acid | adipic acid |
| phosphoric acid | sebacic acid |
| ethanesulfonic acid | (+)-camphoric acid |
| glutamic acid | acetic acid |
| pamoic (embonic) acid | nicotinic acid |
| glutaric acid, 2-oxo- | isobutyric acid |
| 2-naphthoic acid, 1-hydroxy | propionic acid |
| malonic acid | lauric acid |
| gentisic acid | stearic acid |
| L-tartaric acid | orotic acid |
| fumaric acid | carbonic acid |
| galactaric (mucic) acid | |

The acid salts of 4-bromo-2,5-dimethoxyphenethylamine disclosed herein can have any suitable stoichiometric ratio of acid to 4-bromo-2,5-dimethoxyphenethylamine. In one embodiment, the molar ratio of acid is from about 0.4 molar equivalent to about 2.2 molar equivalent, such as forms wherein the salt has a stoichiometric ratio of from about 0.5 molar equivalent to about 2 molar equivalent, such as 0.5, 1 molar equivalent or 2 molar equivalents of the acid.

In some embodiments, the 2C-B salts possess at least one of the following characteristics:

(a) exhibit high crystallinity,

(b) exhibit acceptable scale-up

(c) exhibit minimal water uptake over RH range,

(d) exhibit uncomplicated DSC data (one endothermic event observed), and

(e) acceptable solubility when compared to the other salts.

In some embodiments, the 2C-B salt possess at least one of characteristic as provided in Table 86.

### 2C-B.HCl Salt

In some embodiments, the 2C-B.HCl salt is crystalline polymorphic (Form A) characterized by two or more, or three XRPD signals selected from the group consisting of 5.7 °2θ, 24.1 °2θ, and 23.4 °2θ (±0.2 °2θ; ±0.1 °2θ; or ±0.0 °2θ; Cu Kα1 radiation).

In some embodiments, the 2C-B.HCl salt is crystalline polymorphic (Form A) characterized by two or more, or three XRPD signals selected from the group consisting of 5.7 °2θ, 24.1 °2θ, 23.4 °2θ, and 16.9 °2θ (±0.2 °2θ; ±0.1 °2θ; or ±0.0 °2θ; Cu Kα1 radiation).

In some embodiments, the 2C-B.HCl salt is crystalline polymorphic (Form A) characterized by two or more, or three XRPD signals selected from the group consisting of 5.7 °2θ, 24.1 °2θ, 23.4 °2θ, 16.9 °2θ, and 23.1 °2θ (±0.2 °2θ; ±0.1 °2θ; or ±0.0 °2θ; Cu Kα1 radiation).

In some embodiments, the 2C-B.HCl salt is crystalline polymorphic (Form A) characterized by two or more, or three XRPD signals selected from the group consisting of 5.7 °2θ, 24.1 °2θ, 23.4 °2θ, 16.9 °2θ, 23.1 °2θ, 30.7 °2θ, 20.6 °2θ, 27.5 °2θ, 25.9 °2θ, and 34.2 °2θ (±0.2 °2θ; ±0.1 °2θ; or ±0.0 °2θ; Cu Kα1 radiation).

In some embodiments, the 2C-B.HCl salt is crystalline polymorphic (Form A) characterized by two, three, four, five, six, seven, eight, nine, or ten XRPD signals as shown in Table 6.

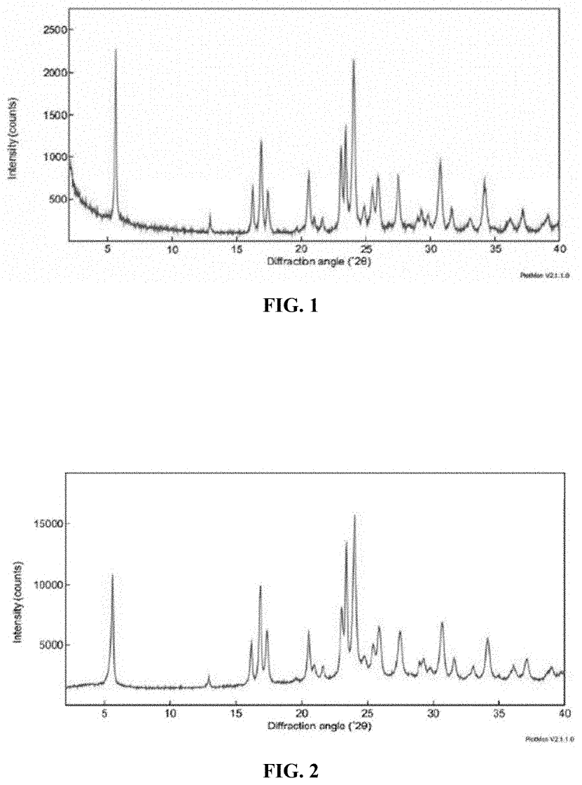

In some embodiments, the 2C-B.HCl salt is crystalline polymorphic (Form A) characterized by two or more, or three XRPD signals as shown in FIG. 1.

In some embodiments, the 2C-B.HCl salt is crystalline polymorphic (Form A) characterized by two or more, or three XRPD signals selected from the group consisting of 24 °2θ, 23.4 °2θ, and 5.6 °2θ (±0.2 °2θ; ±0.1 °2θ; or ±0.0 °2θ; Cu Kα1 radiation).

In some embodiments, the 2C-B.HCl salt is crystalline polymorphic (Form A) characterized by two or more, or three XRPD signals selected from the group consisting of 24 °2θ, 23.4 °2θ, 5.6 °2θ, and 16.8 °2θ (±0.2 °2θ; ±0.1 °2θ; or ±0.0 °2θ; Cu Kα1 radiation).

In some embodiments, the 2C-B.HCl salt is crystalline polymorphic (Form A) characterized by two or more, or three XRPD signals selected from the group consisting of 24 °2θ, 23.4 °2θ, 5.6 °2θ, 16.8 °2θ, and 23 °2θ (±0.2 °2θ; ±0.1 °2θ; or ±0.0 °2θ; Cu Kα1 radiation).

In some embodiments, the 2C-B.HCl salt is crystalline polymorphic (Form A) characterized by two or more, or three XRPD signals selected from the group consisting of 24 °2θ, 23.4 °2θ, 5.6 °2θ, 16.8 °2θ, 23 °2θ, 30.7 °2θ, 25.9 °2θ, 17.4 °2θ, 20.5 °2θ, and 27.4 °2θ (±0.2 °2θ; ±0.1 °2θ; or ±0.0 °2θ; Cu Kα1 radiation).

In some embodiments, the 2C-B.HCl salt is crystalline polymorphic (Form A) characterized by two, three, four, five, six, seven, eight, nine, or ten XRPD signals as shown in Table 7.

In some embodiments, the 2C-B.HCl salt is crystalline polymorphic (Form A) characterized by two or more, or three XRPD signals as shown in FIG. 2.

In some embodiments, the 2C-B.HCl salt is crystalline polymorphic (Form A) characterized by two or more, or three XRPD signals selected from the group consisting of 5.6 °2θ, 16.9 °2θ, and 34.2 °2θ (±0.2 °2θ; ±0.1 °2θ; or ±0.0 °2θ; Cu Kα1 radiation).

In some embodiments, the 2C-B.HCl salt is crystalline polymorphic (Form A) characterized by two or more, or three XRPD signals selected from the group consisting of 5.6 °2θ, 16.9 °2θ, 34.2 °2θ, and 28.4 °2θ (±0.2 °2θ; ±0.1 °2θ; or ±0.0 °2θ; Cu Kα1 radiation). In some embodiments, the 2C-B.HCl salt is crystalline polymorphic (Form A) characterized by two or more, or three XRPD signals selected from the group consisting of 5.6 °2θ, 16.9 °2θ, 34.2 °2θ, 28.4 °2θ, and 22.6 °2θ (±0.2 °2θ; ±0.1 °2θ; or ±0.0 °2θ; Cu Kα1 radiation).

In some embodiments, the 2C-B.HCl salt is crystalline polymorphic (Form A) characterized by two or more, or three XRPD signals selected from the group consisting of 5.6 °2θ, 16.9 °2θ, 34.2 °2θ, 28.4 °2θ, 22.6 °2θ, 30.8 °2θ, 11.3 °2θ, 25.5 °2θ, 20.6 °2θ, and 23.3 °2θ (±0.2 °2θ; ±0.1 °2θ; or ±0.0 °2θ; Cu Kα1 radiation).

In some embodiments, the 2C-B.HCl salt is crystalline polymorphic (Form A) characterized by two or more, or three XRPD signals selected from the group consisting of 5.6 °2θ, 11.3 °2θ, and 16.9 °2θ (±0.2 °2θ; ±0.1 °2θ; or ±0.0 °2θ; Cu Kα1 radiation).

In some embodiments, the 2C-B.HCl salt is crystalline polymorphic (Form A) characterized by two or more, or three XRPD signals selected from the group consisting of 5.6 °2θ, 11.3 °2θ, 16.9 °2θ, 22.6 °2θ and 28.4 °2θ (±0.2 °2θ; ±0.1 °2θ; or ±0.0 °2θ; Cu Kα1 radiation).

In some embodiments, the 2C-B.HCl salt is crystalline polymorphic (Form A) characterized by two or more, or three XRPD signals selected from the group consisting of 5.6 °2θ, 16.8 °2θ, and 17.4 °2θ (±0.2 °2θ; ±0.1 °2θ; or ±0.0 °2θ; Cu Kα1 radiation).

In some embodiments, the 2C-B.HCl salt is crystalline polymorphic (Form A) characterized by two or more, or three XRPD signals selected from the group consisting of 5.6 °2θ, 16.8 °2θ, and 17.4 °2θ, 16.2 °2θ and 24 °2θ (±0.2 °2θ; ±0.1 °2θ; or ±0.0 °2θ; Cu Kα1 radiation).

In some embodiments, the 2C-B.HCl salt is crystalline polymorphic (Form A) characterized by two or more, or three XRPD signals selected from the group consisting of 5.7 °2θ, 16.9 °2θ, and 17.4 °2θ (±0.2 °2θ; ±0.1 °2θ; or ±0.0 °2θ; Cu Kα1 radiation).

In some embodiments, the 2C-B.HCl salt is crystalline polymorphic (Form A) characterized by two or more, or three XRPD signals selected from the group consisting of 5.7 °2θ, 16.9 °2θ, and 17.4 °2θ, 16.2 °2θ and 24.1 °2θ (±0.2 °2θ; ±0.1 °2θ; or ±0.0 °2θ; Cu Kα1 radiation).

In some embodiments, the 2C-B.HCl salt is crystalline polymorphic (Form A) characterized by two, three, four, five, six, seven, eight, nine, or ten XRPD signals as shown in Table 12.

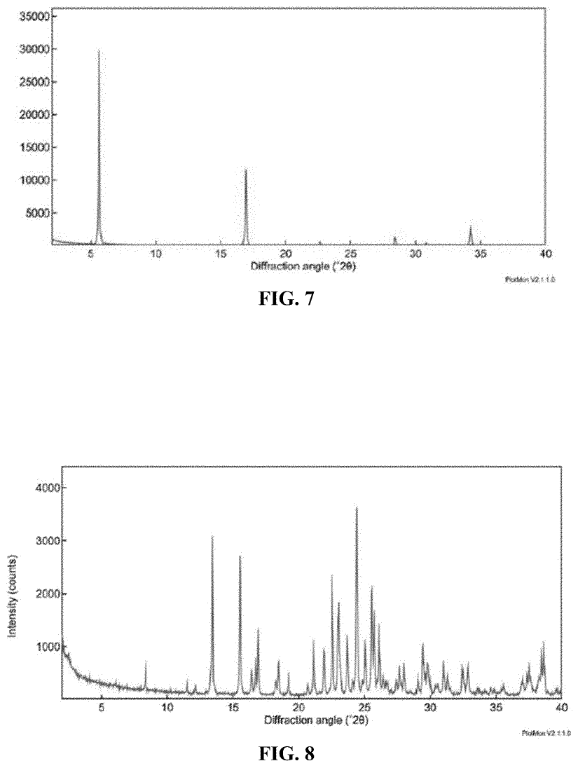

In some embodiments, the 2C-B.HCl salt is crystalline polymorphic (Form A) characterized by two or more, or three XRPD signals as shown in FIG. 7.

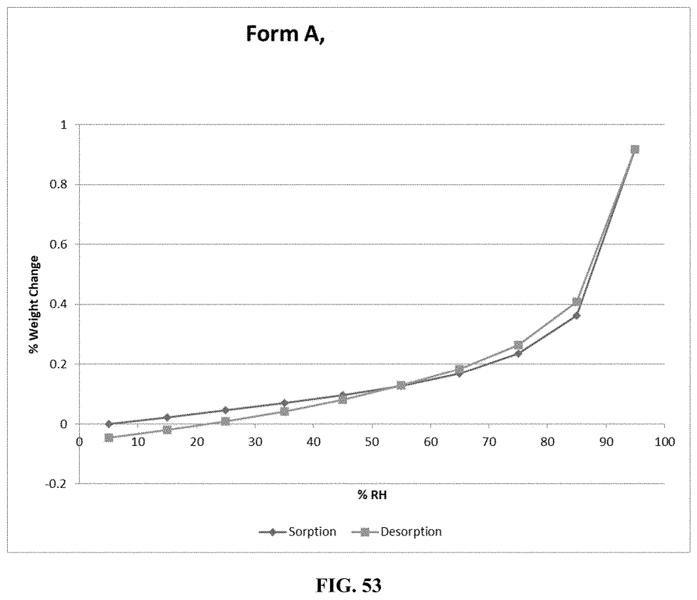

In some embodiments, 2C-B.HCl (Form A) has a DVS profile as provided in FIG. 53.

In some embodiments, the 2C-B.HCl salt is crystalline polymorphic (Form A) characterized by two or more, or three XRPD signals selected from the group consisting of 5.7 °2θ, 24.1 °2θ, and 23.4 °2θ (±0.2 °2θ; ±0.1 °2θ; or ±0.0 °2θ; Cu Kα1 radiation).

In some embodiments, the 2C-B.HCl salt is crystalline polymorphic (Form A) characterized by two or more, or three XRPD signals selected from the group consisting of 5.7 °2θ, 24.1 °2θ, 23.4 °2θ, and 23.1 °2θ (±0.2 °2θ; ±0.1 °2θ; or ±0.0 °2θ; Cu Kα1 radiation).

In some embodiments, the 2C-B.HCl salt is crystalline polymorphic (Form A) characterized by two or more, or three XRPD signals selected from the group consisting of 5.7 °2θ, 24.1 °2θ, 23.4 °2θ, 23.1 °2θ, and 27.5 °2θ (±0.2 °2θ; ±0.1 °2θ; or ±0.0 °2θ; Cu Kα1 radiation).

In some embodiments, the 2C-B.HCl salt is crystalline polymorphic (Form A) characterized by two or more, or three XRPD signals selected from the group consisting of 5.7 °2θ, 24.1 °2θ, 23.4 °2θ, 23.1 °2θ, 27.5 °2θ, 16.9 °2θ, 17 °2θ, 20.6 °2θ, 30.8 °2θ, and 17.4 °2θ (±0.2 °2θ; ±0.1 °2θ; or ±0.0 °2θ; Cu Kα1 radiation).

In some embodiments, the 2C-B.HCl salt is crystalline polymorphic (Form A) characterized by two, three, four, five, six, seven, eight, nine, or ten XRPD signals as shown in Table 58.

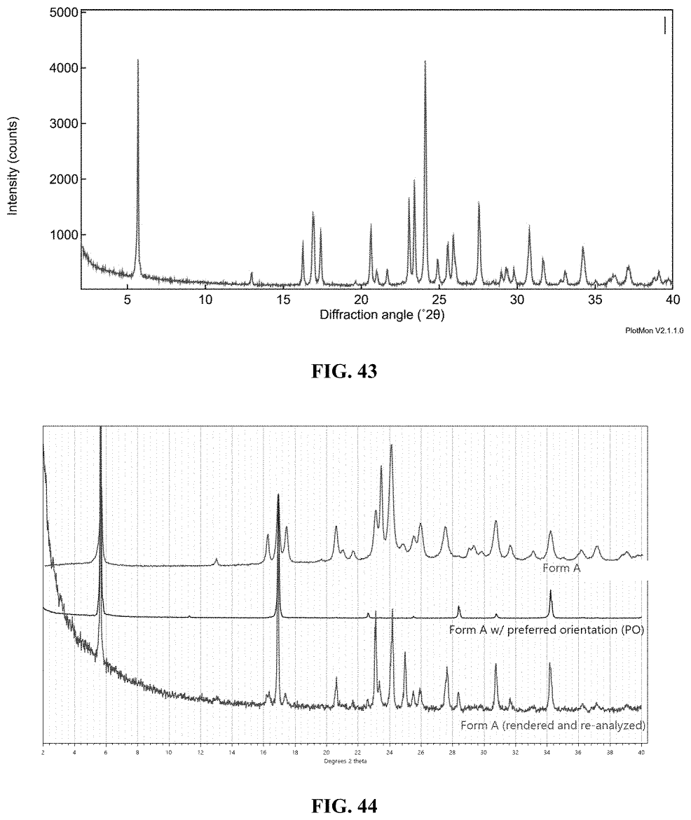

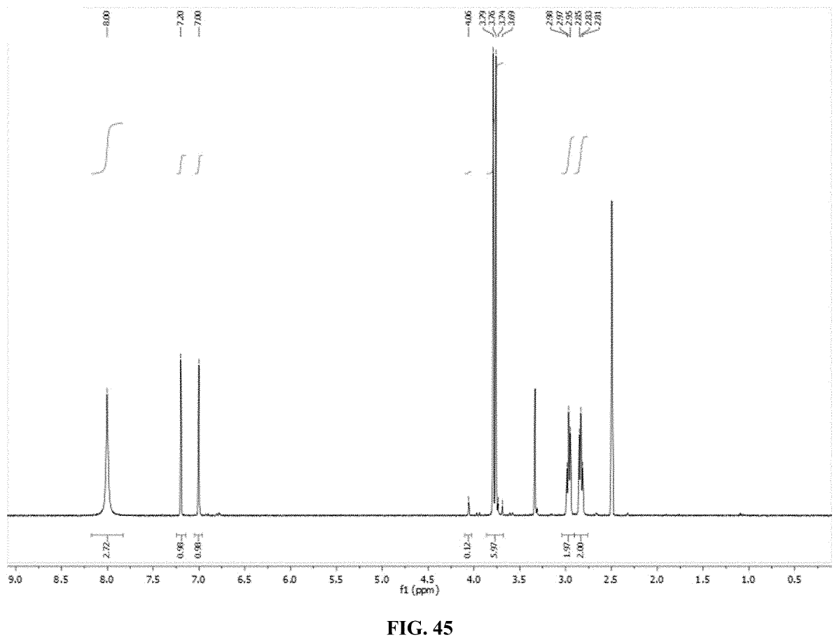

In some embodiments, the 2C-B.HCl salt is crystalline polymorphic (Form A) characterized by two or more, or three XRPD signals as shown in FIG. 43.

In some embodiments, the 2C-B.HCl salt is crystalline polymorphic (Form A) characterized by two or more, or three XRPD signals selected from the group consisting of 5.7 °2θ, 16.9 °2θ, and 17.4 °2θ (±0.2 °2θ; ±0.1 °2θ; or ±0.0 °2θ; Cu Kα1 radiation).

In some embodiments, the 2C-B.HCl salt is crystalline polymorphic (Form A) characterized by two or more, or three XRPD signals selected from the group consisting of 5.7 °2θ, 16.9 °2θ, 17.4 °2θ, 16.3 °2θ, and 24.1 °2θ (±0.2 °2θ; ±0.1 °2θ; or ±0.0 °2θ; Cu Kα1 radiation).

In some embodiments, the 2C-B.HCl salt is crystalline polymorphic (Form B) characterized by two or more, or three XRPD signals selected from the group consisting of 5.5 °2θ, 16.6 °2θ, and 30.2 °2θ (±0.2 °2θ; ±0.1 °2θ; or ±0.0 °2θ; Cu Kα1 radiation).

In some embodiments, the 2C-B.HCl salt is crystalline polymorphic (Form B) characterized by two or more, or three XRPD signals selected from the group consisting of 5.5 °2θ, 16.6 °2θ, 30.2 °2θ, and 33.5 °2θ (±0.2 °2θ; ±0.1 °2θ; or ±0.0 °2θ; Cu Kα1 radiation).

In some embodiments, the 2C-B.HCl salt is crystalline polymorphic (Form B) characterized by two or more, or three XRPD signals selected from the group consisting of 5.5 °2θ, 16.6 °2θ, 30.2 °2θ, 33.5 °2θ, and 34 °2θ (±0.2 °2θ; ±0.1 °2θ; or ±0.0 °2θ; Cu Kα1 radiation).

In some embodiments, the 2C-B.HCl salt is crystalline polymorphic (Form B) characterized by two or more, or three XRPD signals selected from the group consisting of 5.5 °2θ, 16.6 °2θ, 30.2 °2θ, 33.5 °2θ, 34 °2θ, 5 °2θ, 23 °2θ, 23.4 °2θ, 23.8 °2θ, and 16 °2θ (±0.2 °2θ; ±0.1 °2θ; or ±0.0 °2θ; Cu Kα1 radiation).

In some embodiments, the 2C-B.HCl salt is crystalline polymorphic (Form B) characterized by two, three, four, five, six, seven, eight, nine, or ten XRPD signals as shown in Table 8.

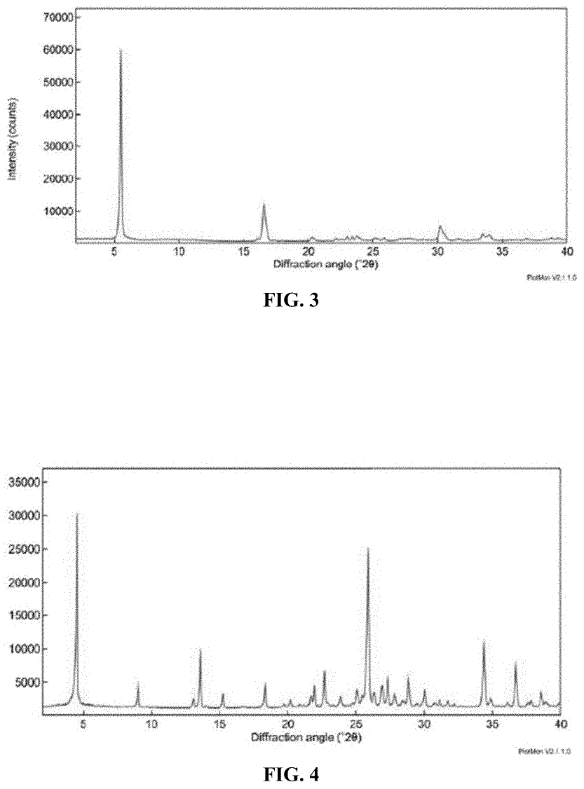

In some embodiments, the 2C-B.HCl salt is crystalline polymorphic (Form B) characterized by two or more, or three XRPD signals as shown in FIG. 3.

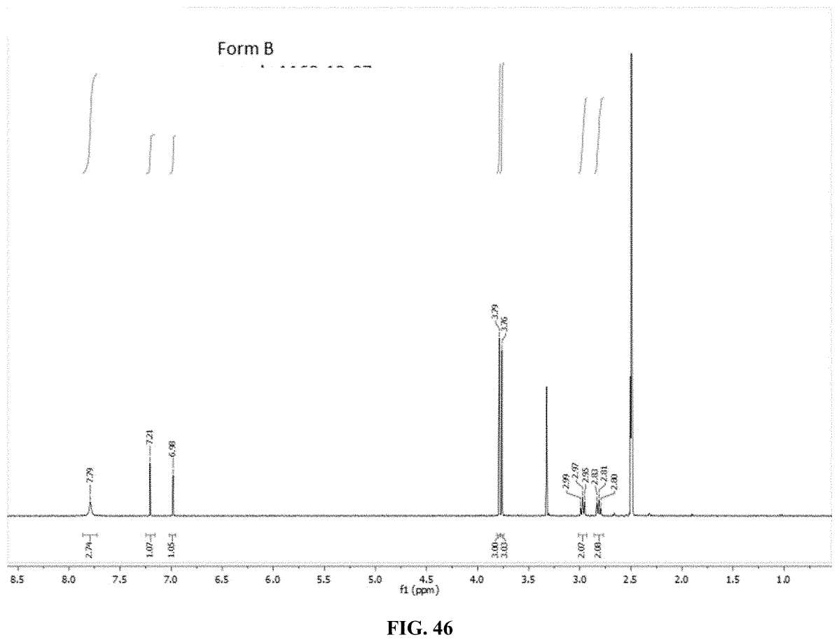

In some embodiments, 2C-B.HCl (Form B) has an ¹H NMR spectra as provided in FIG. 46.

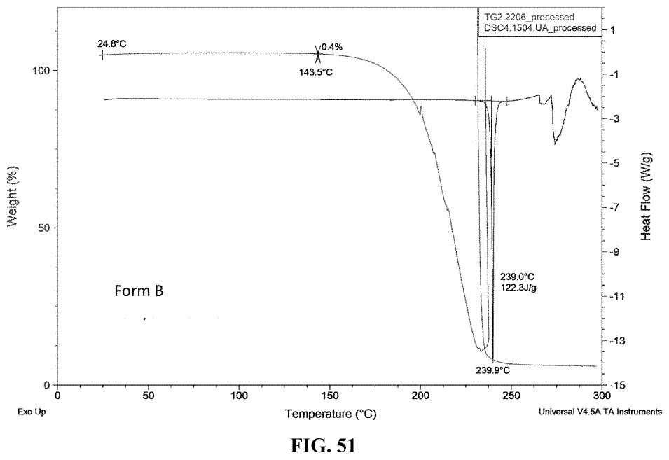

In some embodiments, 2C-B.HCl (Form B) has TGA and DSC profiles as provided in FIG. 51.

In some embodiments, the 2C-B.HCl salt is crystalline polymorphic (Form B) characterized by two or more, or three XRPD signals selected from the group consisting of 23.6 °2θ, 5.6 °2θ, and 30.3 °2θ (±0.2 °2θ; ±0.1 °2θ; or ±0.0 °2θ; Cu Kα1 radiation).

In some embodiments, the 2C-B.HCl salt is crystalline polymorphic (Form B) characterized by two or more, or three XRPD signals selected from the group consisting of 23.6 °2θ, 5.6 °2θ, 30.3 °2θ, and 16.2 °2θ (±0.2 °2θ; ±0.1 °2θ; or ±0.0 °2θ; Cu Kα1 radiation).

In some embodiments, the 2C-B.HCl salt is crystalline polymorphic (Form B) characterized by two or more, or three XRPD signals selected from the group consisting of 23.6 °2θ, 5.6 °2θ, 30.3 °2θ, 16.2 °2θ, and 20.4 °2θ (±0.2 °2θ; ±0.1 °2θ; or ±0.0 °2θ; Cu Kα1 radiation).

In some embodiments, the 2C-B.HCl salt is crystalline polymorphic (Form B) characterized by two or more, or three XRPD signals selected from the group consisting of 23.6 °2θ, 5.6 °2θ, 30.3 °2θ, 16.2 °2θ, 20.4 °2θ, 26.1 °2θ, 16.6 °2θ, 22.7 °2θ, 25.0 °2θ, and 36.6 °2θ (±0.2 °2θ; ±0.1 °2θ; or ±0.0 °2θ; Cu Kα1 radiation).

In some embodiments, the 2C-B.HCl salt is crystalline polymorphic (Form B) characterized by two, three, four, five, six, seven, eight, nine, or ten XRPD signals as shown in Table 57.

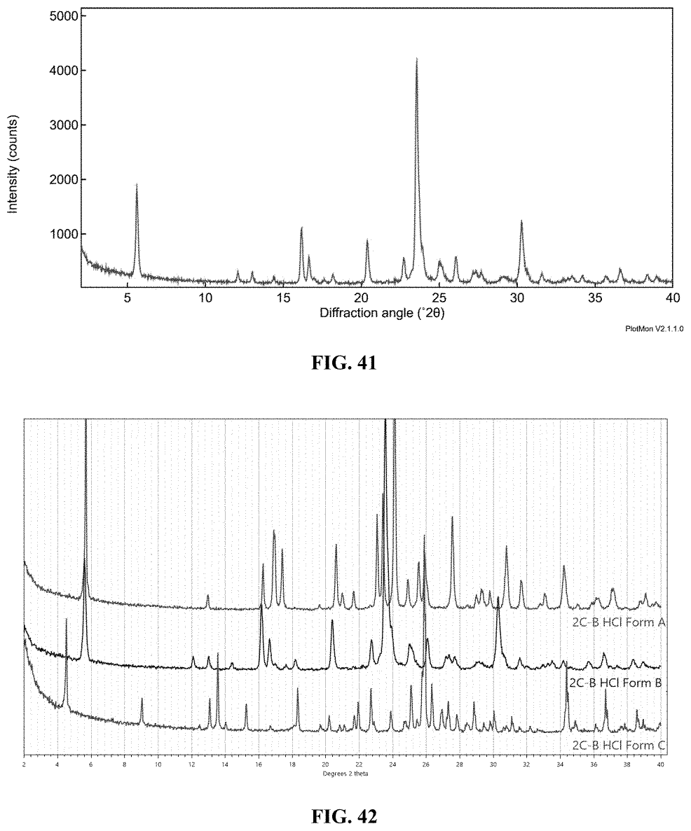

In some embodiments, the 2C-B.HCl salt is crystalline polymorphic (Form B) characterized by two or more, or three XRPD signals as shown in FIG. 41.

In some embodiments, the 2C-B.HCl salt is crystalline polymorphic (Form B) characterized by two or more, or three XRPD signals selected from the group consisting of 5.6 °2θ, 16.2 °2θ, and 16.6 °2θ (±0.2 °2θ; ±0.1 °2θ; or ±0.0 °2θ; Cu Kα1 radiation).

In some embodiments, the 2C-B.HCl salt is crystalline polymorphic (Form B) characterized by two or more, or three XRPD signals selected from the group consisting of 5.6 °2θ, 16.2 °2θ, 16.6 °2θ, 23.6 °2θ, and 30.3 °2θ (±0.2 °2θ; ±0.1 °2θ; or ±0.0 °2θ; Cu Kα1 radiation).

In some embodiments, the 2C-B.HCl salt is crystalline polymorphic (Form B) characterized by two or more, or three XRPD signals selected from the group consisting of 5.5 °2θ, 16.0 °2θ, and 16.6 °2θ (±0.2 °2θ; ±0.1 °2θ; or ±0.0 °2θ; Cu Kα1 radiation).

In some embodiments, the 2C-B.HCl salt is crystalline polymorphic (Form B) characterized by two or more, or three XRPD signals selected from the group consisting of 5.5 °2θ, 16.0 °2θ, 16.6 °2θ, 23.4 °2θ, and 30.2 °2θ (±0.2 °2θ; ±0.1 °2θ; or ±0.0 °2θ; Cu Kα1 radiation).

In some embodiments, the 2C-B.HCl is crystalline polymorphic (Form C) characterized by two or more, or three XRPD signals selected from the group consisting of 4.5 °2θ, 25.9 °2θ, and 34.4 °2θ (±0.2 °2θ; ±0.1 °2θ; or ±0.0 °2θ; Cu Kα1 radiation).

In some embodiments, the 2C-B.HCl is crystalline polymorphic (Form C) characterized by two or more, or three XRPD signals selected from the group consisting of 4.5 °2θ, 25.9 °2θ, 34.4 °2θ, and 13.6 °2θ (±0.2 °2θ; ±0.1 °2θ; or ±0.0 °2θ; Cu Kα1 radiation).

In some embodiments, the 2C-B.HCl is crystalline polymorphic (Form C) characterized by two or more, or three XRPD signals selected from the group consisting of 4.5 °2θ, 25.9 °2θ, 34.4 °2θ, 13.6 °2θ, and 36.7 °2θ (±0.2 °2θ; ±0.1 °2θ; or ±0.0 °2θ; Cu Kα1 radiation).

In some embodiments, the 2C-B.HCl is crystalline polymorphic (Form C) characterized by two or more, or three XRPD signals selected from the group consisting of 4.5 °2θ, 25.9 °2θ, 34.4 °2θ, 13.6 °2θ, 36.7 °2θ, 22.7 °2θ, 27.3 °2θ, 28.8 °2θ, 9 °2θ, and 18.3 °2θ (±0.2 °2θ; ±0.1 °2θ; or ±0.0 °2θ; Cu Kα1 radiation).

In some embodiments, the 2C-B.HCl (Form C) is a crystalline polymorph characterized by two or more, or three XRPD signals selected from the group consisting of 4.5 °2θ, 9.0 °2θ, and 13.6 °2θ (±0.2 °2θ; ±0.1 °2θ; or ±0.0 °2θ; Cu Kα1 radiation).

In some embodiments, the 2C-B.HCl (Form C) is a crystalline polymorph characterized by two or more, or three XRPD signals selected from the group consisting of 4.5 °2θ, 9.0 °2θ, 13.6 °2θ, 18.3 °2θ, and 25.9 °2θ (±0.2 °2θ; ±0.1 °2θ; or ±0.0 °2θ; Cu Kα1 radiation).

In some embodiments, the 2C-B.HCl is crystalline polymorphic (Form C) characterized by two, three, four, five, six, seven, eight, nine, or ten XRPD signals as shown in Table 9.

In some embodiments, the 2C-B.HCl is crystalline polymorphic (Form C) characterized by two or more, or three XRPD signals as shown in FIG. 4.

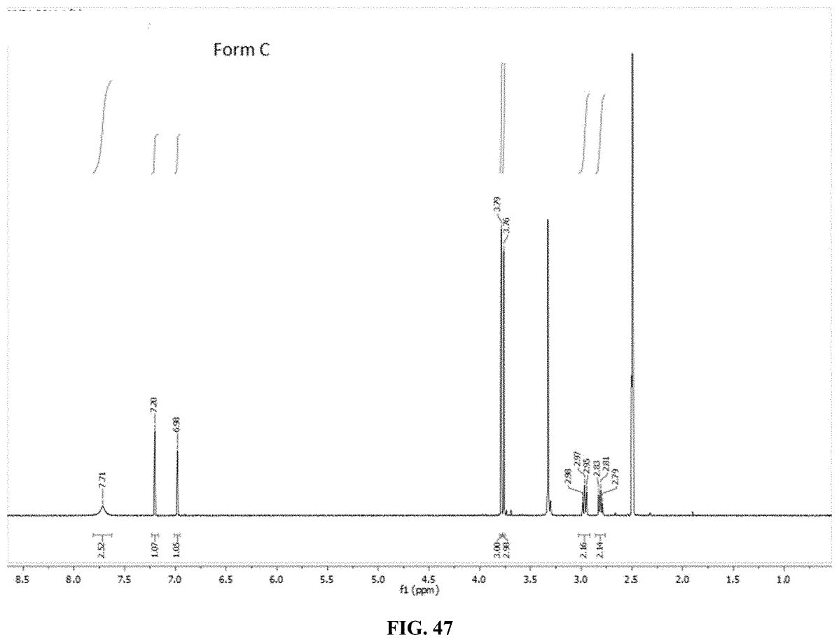

In some embodiments, 2C-B.HCl (Form C) has an ¹H NMR spectra as provided in FIG. 47.

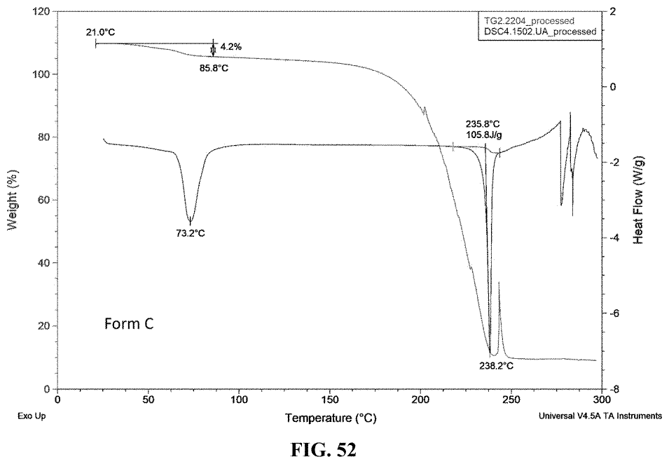

In some embodiments, 2C-B.HCl (Form C) has TGA and DSC profiles as provided in FIG. 52.

2C-B B.esylate Salt

In some embodiments, the 2C-B besylate salt is crystalline polymorphic (Form 1) characterized by two or more, or three XRPD signals selected from the group consisting of 24.4 °2θ, 13.4 °2θ, and 15.5 °2θ (±0.2 °2θ; ±0.1 °2θ; or ±0.0 °2θ; Cu Kα1 radiation).

In some embodiments, the 2C-B besylate salt is crystalline polymorphic (Form 1) characterized by two or more, or three XRPD signals selected from the group consisting of 24.4 °2θ, 13.4 °2θ, 15.5 °2θ, and 22.5 °2θ (±0.2 °2θ; ±0.1 °2θ; or ±0.0 °2θ; Cu Kα1 radiation).

In some embodiments, the 2C-B besylate salt is crystalline polymorphic (Form 1) characterized by two or more, or three XRPD signals selected from the group consisting of 24.4 °2θ, 13.4 °2θ, 15.5 °2θ, 22.5 °2θ, and 25.5 °2θ (±0.2 °2θ; ±0.1 °2θ; or ±0.0 °2θ; Cu Kα1 radiation).

In some embodiments, the 2C-B besylate salt is crystalline polymorphic (Form 1) characterized by two or more, or three XRPD signals selected from the group consisting of 24.4 °2θ, 13.4 °2θ, 15.5 °2θ, 22.5 °2θ, 25.5 °2θ, 23 °2θ, 25.7 °2θ, 26.1 °2θ, 16.9 °2θ, and 23.7 °2θ (±0.2 °2θ; ±0.1 °2θ; or ±0.0 °2θ; Cu Kα1 radiation).

In some embodiments, the 2C-B besylate salt is crystalline polymorphic (Form 1) characterized by two or more, or three XRPD signals selected from the group consisting of 8.4 °2θ, 11.5 °2θ, and 13.4 °2θ (±0.2 °2θ; ±0.1 °2θ; or ±0.0 °2θ; Cu Kα1 radiation).

In some embodiments, the 2C-B besylate salt is crystalline polymorphic (Form 1) characterized by two or more, or three XRPD signals selected from the group consisting of 8.4 °2θ, 11.5 °2θ, 13.4 °2θ, 15.5 °2θ, and 16.7 °2θ (±0.2 °2θ; ±0.1 °2θ; or ±0.0 °2θ; Cu Kα1 radiation).

In some embodiments, the 2C-B besylate salt (Form 1) characterized by two, three, four, five, six, seven, eight, nine, or ten XRPD signals as shown in Table 19. In some embodiments, the 2C-B besylate salt (Form 1) characterized by two or more, or three XRPD signals as shown in FIG. 8.

In some embodiments, the 2C-B B.esylate (Form 1) is a crystalline polymorph characterized by two or more, or three XRPD signals selected from the group consisting of 8.4 °2θ, 12.1 °2θ, and 13.4 °2θ (±0.2 °2θ; ±0.1 °2θ; or ±0.0 °2θ; Cu Kα1 radiation).

In some embodiments, the 2C-B B.esylate (Form 1) is a crystalline polymorph characterized by two or more, or three XRPD signals selected from the group consisting of 8.4 °2θ, 12.1 °2θ, 13.4 °2θ, 15.5 °2θ, and 16.7 °2θ (±0.2 °2θ; ±0.1 °2θ; or ±0.0 °2θ; Cu Kα1 radiation).

In some embodiments, the 2C-B besylate salt is crystalline polymorphic (Form 1) characterized by two or more, or three XRPD signals selected from the group consisting of 13.4 °2θ, 24.4 °2θ, and 25.7 °2θ (±0.2 °2θ; ±0.1 °2θ; or ±0.0 °2θ; Cu Kα1 radiation).

In some embodiments, the 2C-B besylate salt is crystalline polymorphic (Form 1) characterized by two or more, or three XRPD signals selected from the group consisting of 13.4 °2θ, 24.4 °2θ, 25.7 °2θ, and 25 °2θ (±0.2 °2θ; ±0.1 °2θ; or ±0.0 °2θ; Cu Kα1 radiation).

In some embodiments, the 2C-B besylate salt is crystalline polymorphic (Form 1) characterized by two or more, or three XRPD signals selected from the group consisting of 13.4 °2θ, 24.4 °2θ, 25.7 °2θ, 25 °2θ, and 15.5 °2θ (±0.2 °2θ; ±0.1 °2θ; or ±0.0 °2θ; Cu Kα1 radiation).

In some embodiments, the 2C-B besylate salt is crystalline polymorphic (Form 1) characterized by two or more, or three XRPD signals selected from the group consisting of 13.4 °2θ, 24.4 °2θ, 25.7 °2θ, 25 °2θ, 15.5 °2θ, 22.5 °2θ, 23 °2θ, 38.4 °2θ, 38.6 °2θ, and 26.1 °2θ (±0.2 °2θ; ±0.1 °2θ; or ±0.0 °2θ; Cu Kα1 radiation).

In some embodiments, the 2C-B besylate salt (Form 1) characterized by two, three, four, five, six, seven, eight, nine, or ten XRPD signals as shown in Table 61.

In some embodiments, the 2C-B besylate salt (Form 1) characterized by two or more, or three XRPD signals as shown in FIG. 55.

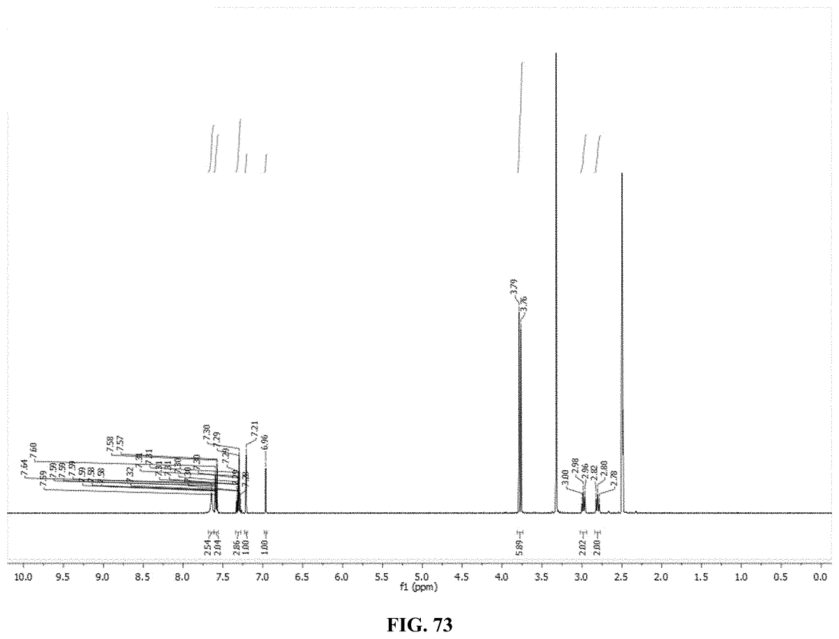

In some embodiments, the 2C-B besylate salt (Form 1) has an ¹H NMR spectra as provided in FIG. 73.

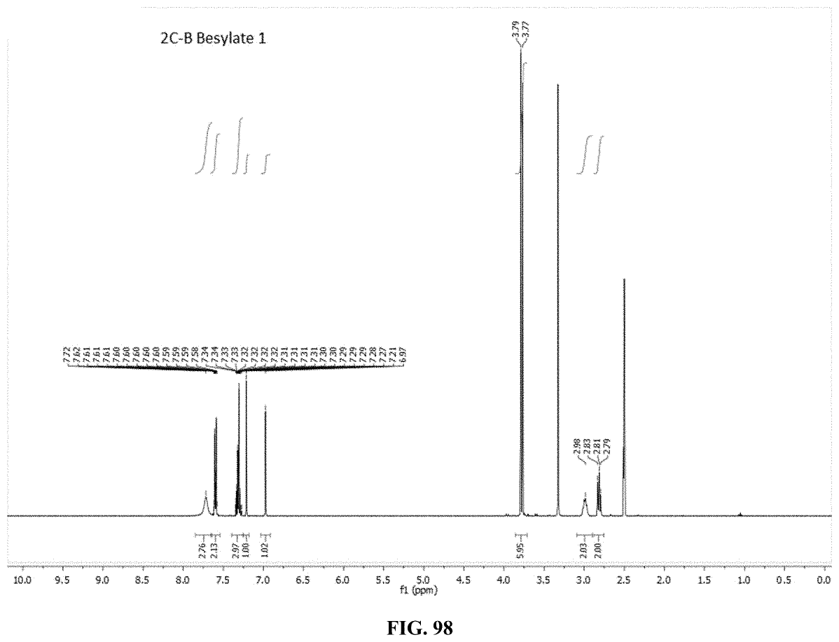

In some embodiments, the 2C-B besylate salt (Form 1) has an ¹H NMR spectra as provided in FIG. 98.

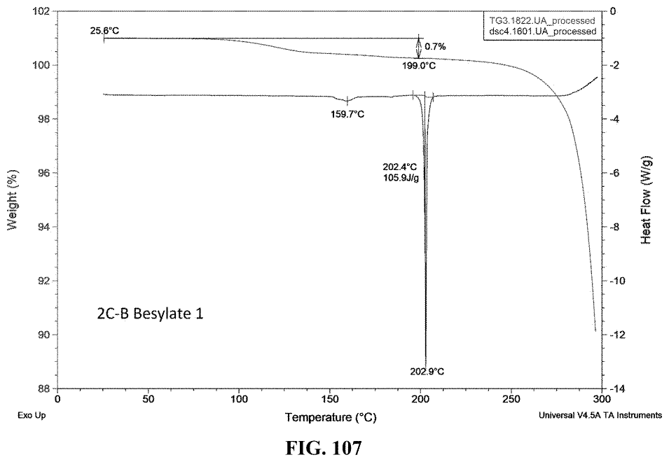

In some embodiments, the 2C-B besylate salt (Form 1) has TGA and DSC profiles as provided in FIG. 107.

In some embodiments, the 2C-B besylate salt (Form 1) has TGA and DSC profiles as provided in FIG. 125.

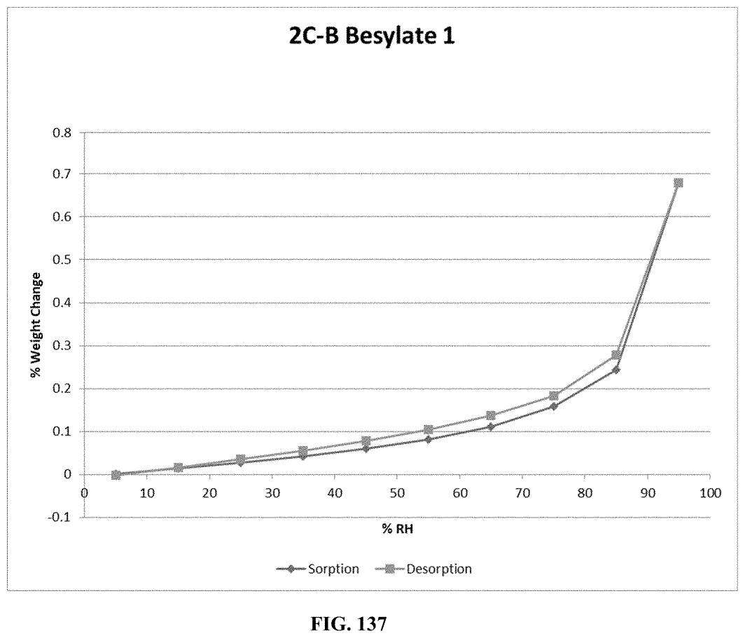

In some embodiments, the 2C-B besylate salt (Form 1) has a DVS profile as provided in FIG. 137.

In some embodiments, the 2C-B besylate salt is crystalline polymorphic (Form 2) characterized by two or more, or three XRPD signals selected from the group consisting of 20 °2θ, 19 °2θ, and 24.1 °2θ (±0.2 °2θ; ±0.1 °2θ; or ±0.0 °2θ; Cu Kα1 radiation).

In some embodiments, the 2C-B besylate salt is crystalline polymorphic (Form 2) characterized by two or more, or three XRPD signals selected from the group consisting of 20 °2θ, 19 °2θ, 24.1 °2θ, and 16.4 °2θ (±0.2 °2θ; ±0.1 °2θ; or ±0.0 °2θ; Cu Kα1 radiation).

In some embodiments, the 2C-B besylate salt is crystalline polymorphic (Form 2) characterized by two or more, or three XRPD signals selected from the group consisting of 20 °2θ, 19 °2θ, 24.1 °2θ, 16.4 °2θ, and 24.9 °2θ (±0.2 °2θ; ±0.1 °2θ; or ±0.0 °2θ; Cu Kα1 radiation).

In some embodiments, the 2C-B besylate salt is crystalline polymorphic (Form 2) characterized by two or more, or three XRPD signals selected from the group consisting of 20 °2θ, 19 °2θ, 24.1 °2θ, 16.4 °2θ, 24.9 °2θ, 22.9 °2θ, 11.4 °2θ, 25.6 °2θ, 17.1 °2θ, and 26.4 °2θ (±0.2 °2θ; ±0.1 °2θ; or ±0.0 °2θ; Cu Kα1 radiation).

In some embodiments, the 2C-B besylate salt (Form 2) characterized by two, three, four, five, six, seven, eight, nine, or ten XRPD signals as shown in Table 38.

In some embodiments, the 2C-B besylate salt (Form 2) characterized by two or more, or three XRPD signals as shown in FIG. 29.

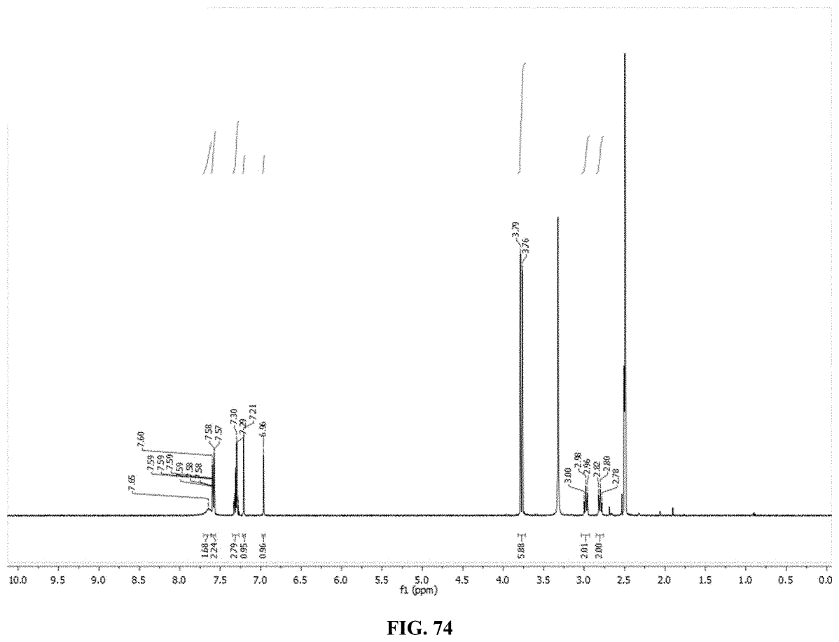

In some embodiments, the 2C-B besylate salt (Form 2) has an ¹H NMR spectra as provided in FIG. 74.

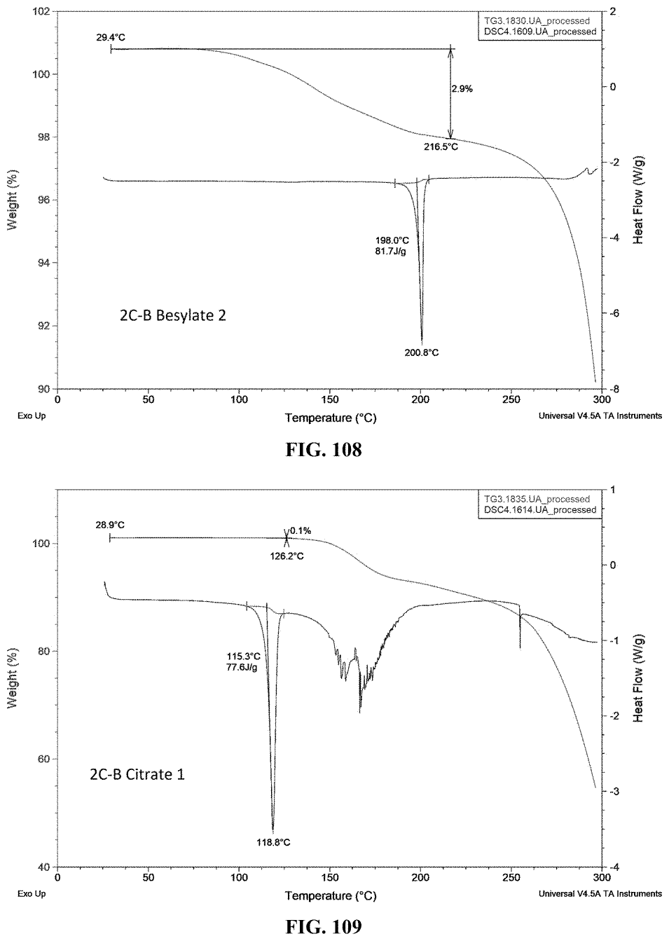

In some embodiments, the 2C-B besylate salt (Form 2) has TGA and DSC profiles as provided in FIG. 108.

In some embodiments, the 2C-B B.esylate (Form 2) is a crystalline polymorph characterized by two or more, or three XRPD signals selected from the group consisting of 5.7 °2θ, 11.4 °2θ, and 19.0 °2θ (±0.2 °2θ; ±0.1 °2θ; or ±0.0 °2θ; Cu Kα1 radiation).

In some embodiments, the 2C-B B.esylate (Form 2) is a crystalline polymorph characterized by two or more, or three XRPD signals selected from the group consisting of 5.7 °2θ, 11.4 °2θ, 19.0 °2θ, 16.4 °2θ, and 20.0 °2θ (±0.2 °2θ; ±0.1 °2θ; or ±0.0 °2θ; Cu Kα1 radiation).

### 2C-B Citrate Salt

In some embodiments, the 2C-B Citrate salt is crystalline polymorphic (Form 1) characterized by two or more, or three XRPD signals selected from the group consisting of 24.7 °2θ, 22.5 °2θ, and 13.7 °2θ (±0.2 °2θ; ±0.1 °2θ; or ±0.0 °2θ; Cu Kα1 radiation).

In some embodiments, the 2C-B Citrate salt is crystalline polymorphic (Form 1) characterized by two or more, or three XRPD signals selected from the group consisting of 24.7 °2θ, 22.5 °2θ, 13.7 °2θ, and 20.2 °2θ (±0.2 °2θ; ±0.1 °2θ; or ±0.0 °2θ; Cu Kα1 radiation).

In some embodiments, the 2C-B Citrate salt is crystalline polymorphic (Form 1) characterized by two or more, or three XRPD signals selected from the group consisting of 24.7 °2θ, 22.5 °2θ, 13.7 °2θ, 20.2 °2θ, and 20.6 °2θ (±0.2 °2θ; ±0.1 °2θ; or ±0.0 °2θ; Cu Kα1 radiation).

In some embodiments, the 2C-B Citrate salt is crystalline polymorphic (Form 1) characterized by two or more, or three XRPD signals selected from the group consisting of 24.7 °2θ, 22.5 °2θ, 13.7 °2θ, 20.2 °2θ, 20.6 °2θ, 22.4 °2θ, 22.7 °2θ, 14.2 °2θ, 27.9 °2θ, and 13.4 °2θ (±0.2 °2θ; ±0.1 °2θ; or ±0.0 °2θ; Cu Kα1 radiation).

In some embodiments, the 2C-B Citrate salt is crystalline polymorphic (Form 1) characterized by two, three, four, five, six, seven, eight, nine, or ten XRPD signals as shown in Table 20.

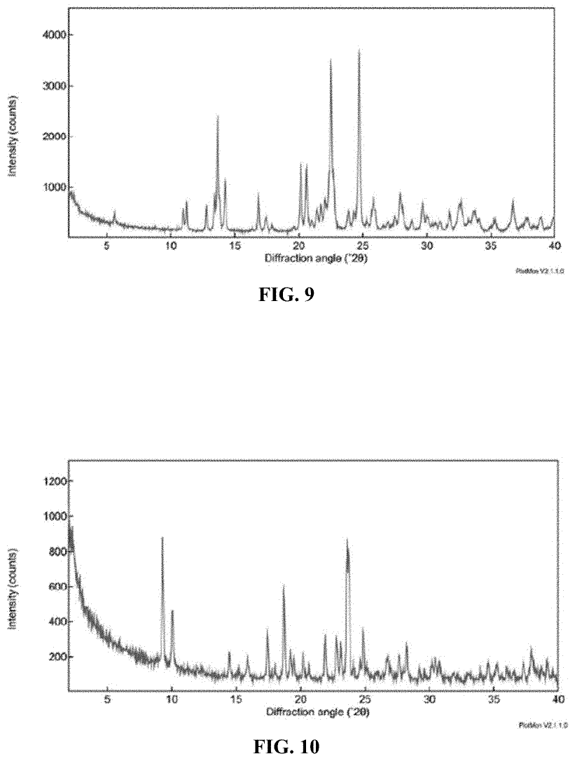

In some embodiments, the 2C-B Citrate salt is crystalline polymorphic (Form 1) characterized by two or more, or three XRPD signals as shown in FIG. 9.

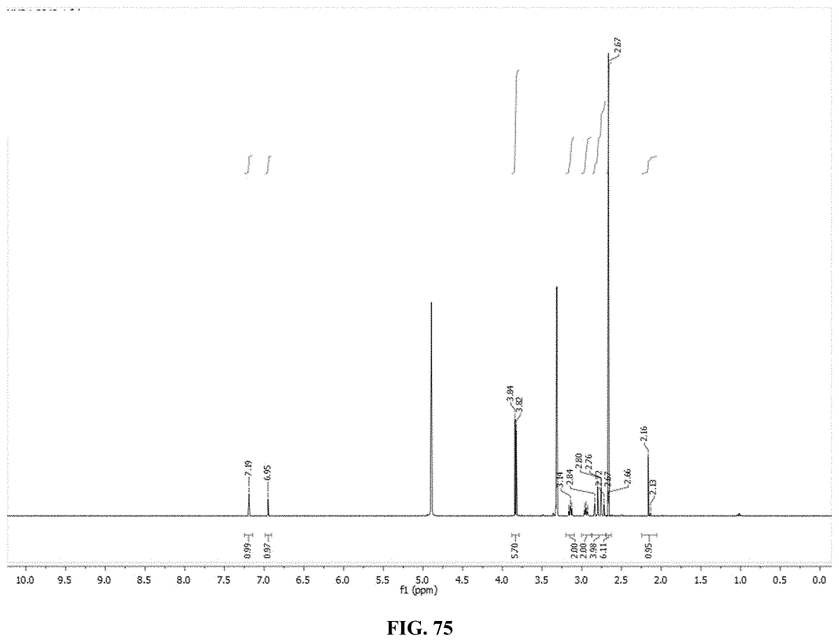

In some embodiments, 2C-B Citrate salt (Form 1) has an ¹H NMR spectra as provided in FIG. 75.

In some embodiments, 2C-B Citrate salt (Form 1) has TGA and DSC profiles as provided in FIG. 109.

In some embodiments, the 2C-B citrate (Form 1) is a crystalline polymorph characterized by two or more, or three XRPD signals selected from the group consisting of 11.0 °2θ, 11.2 °2θ, and 12.8 °2θ (±0.2 °2θ; ±0.1 °2θ; or ±0.0 °2θ; Cu Kα1 radiation).

In some embodiments, the 2C-B citrate (Form 1) is a crystalline polymorph characterized by two or more, or three XRPD signals selected from the group consisting of 11.0 °2θ, 11.2 °2θ, and 12.8 °2θ, 13.7 °2θ, and 14.2 °2θ (±0.2 °2θ; ±0.1 °2θ; or ±0.0 °2θ; Cu Kα1 radiation).

In some embodiments, the 2C-B Citrate salt is crystalline polymorphic (Form 2) characterized by two or more, or three XRPD signals selected from the group consisting of 20.6 °2θ, 21.8 °2θ, and 16.6 °2θ (±0.2 °2θ; ±0.1 °2θ; or ±0.0 °2θ; Cu Kα1 radiation).

In some embodiments, the 2C-B Citrate salt is crystalline polymorphic (Form 2) characterized by two or more, or three XRPD signals selected from the group consisting of 20.6 °2θ, 21.8 °2θ, 16.6 °2θ, and 24.4 °2θ (±0.2 °2θ; ±0.1 °2θ; or ±0.0 °2θ; Cu Kα1 radiation).

In some embodiments, the 2C-B Citrate salt is crystalline polymorphic (Form 2) characterized by two or more, or three XRPD signals selected from the group consisting of 20.6 °2θ, 21.8 °2θ, 16.6 °2θ, 24.4 °2θ, and 26.8 °2θ (±0.2 °2θ; ±0.1 °2θ; or ±0.0 °2θ; Cu Kα1 radiation).

In some embodiments, the 2C-B Citrate salt is crystalline polymorphic (Form 2) characterized by two or more, or three XRPD signals selected from the group consisting of 20.6 °2θ, 21.8 °2θ, 16.6 °2θ, 24.4 °2θ, 26.8 °2θ, 26.3 °2θ, 14.5 °2θ, 30.2 °2θ, 23.6 °2θ, and 30.2 °2θ (±0.2 °2θ; ±0.1 °2θ; or ±0.0 °2θ; Cu Kα1 radiation).

In some embodiments, the 2C-B Citrate salt is crystalline polymorphic (Form 2) characterized by two, three, four, five, six, seven, eight, nine, or ten XRPD signals as shown in Table 47.

In some embodiments, the 2C-B Citrate salt is crystalline polymorphic (Form 2) characterized by two or more, or three XRPD signals as shown in FIG. 39.

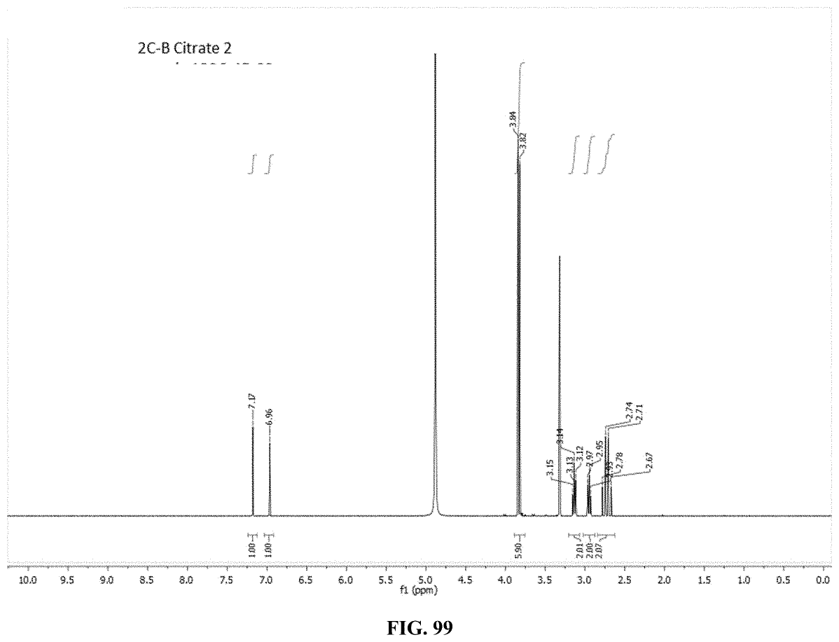

In some embodiments, 2C-B Citrate salt (Form 2) has an ¹H NMR spectra as provided in FIG. 99.

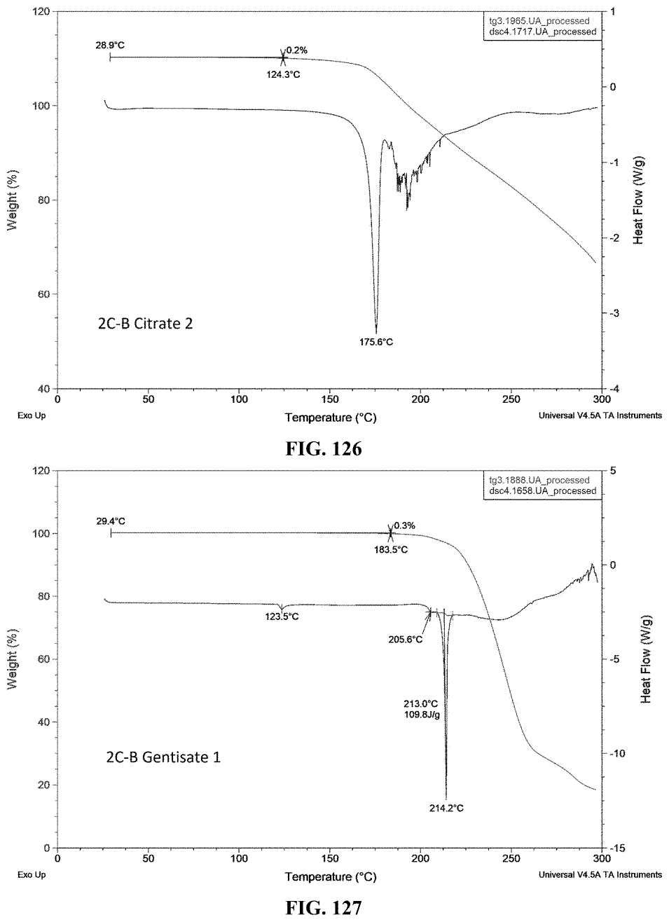

In some embodiments, 2C-B Citrate salt (Form 2) has TGA and DSC profiles as provided in FIG. 126.

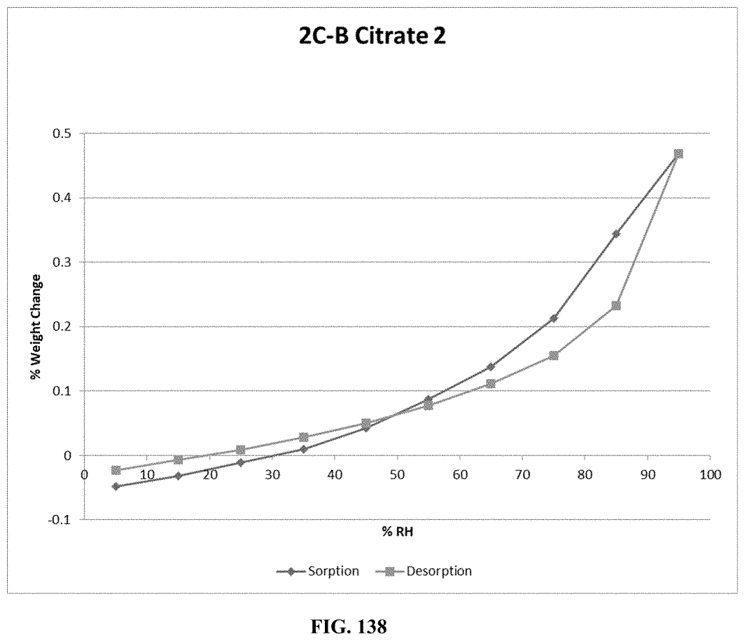

In some embodiments, 2C-B Citrate salt (Form 2) has a DVS profile as provided in FIG. 138.

In some embodiments, the 2C-B Citrate (Form 2) is a crystalline polymorph characterized by two or more, or three XRPD signals selected from the group consisting of 3.7 °2θ, 10.3 °2θ, and 13.1 °2θ (±0.2 °2θ; ±0.1 °2θ; or ±0.0 °2θ; Cu Kα1 radiation).

In some embodiments, the 2C-B Citrate (Form 2) is a crystalline polymorph characterized by two or more, or three XRPD signals selected from the group consisting of 3.7 °2θ, 10.3 °2θ, 13.1 °2θ, 14.5 °2θ, and 16.6 °2θ (±0.2 °2θ; ±0.1 °2θ; or ±0.0 °2θ; Cu Kα1 radiation).

### 2C-B Esylate Salt

In some embodiments, the 2C-B Esylate is crystalline polymorphic (Form 1) characterized by two or more, or three XRPD signals selected from the group consisting of 9.3 °2θ, 23.6 °2θ, and 23.7 °2θ (±0.2 °2θ; ±0.1 °2θ; or ±0.0 °2θ; Cu Kα1 radiation).

In some embodiments, the 2C-B Esylate is crystalline polymorphic (Form 1) characterized by two or more, or three XRPD signals selected from the group consisting of 9.3 °2θ, 23.6 °2θ, 23.7 °2θ, and 18.7 °2θ (±0.2 °2θ; ±0.1 °2θ; or ±0.0 °2θ; Cu Kα1 radiation).

In some embodiments, the 2C-B Esylate is crystalline polymorphic (Form 1) characterized by two or more, or three XRPD signals selected from the group consisting of 9.3 °2θ, 23.6 °2θ, 23.7 °2θ, 18.7 °2θ, and 10 °2θ (±0.2 °2θ; ±0.1 °2θ; or ±0.0 °2θ; Cu Kα1 radiation).

In some embodiments, the 2C-B Esylate is crystalline polymorphic (Form 1) characterized by two or more, or three XRPD signals selected from the group consisting of 9.3 °2θ, 23.6 °2θ, 23.7 °2θ, 18.7 °2θ, 10 °2θ, 24.8 °2θ, 17.4 °2θ, 21.9 °2θ, 22.8 °2θ, and 23.1 °2θ (±0.2 °2θ; ±0.1 °2θ; or ±0.0 °2θ; Cu Kα1 radiation).

In some embodiments, the 2C-B Esylate is crystalline polymorphic (Form 1) characterized by two, three, four, five, six, seven, eight, nine, or ten XRPD signals as shown in Table 21.

In some embodiments, the 2C-B Esylate is crystalline polymorphic (Form 1) characterized by two or more, or three XRPD signals as shown in FIG. 10.

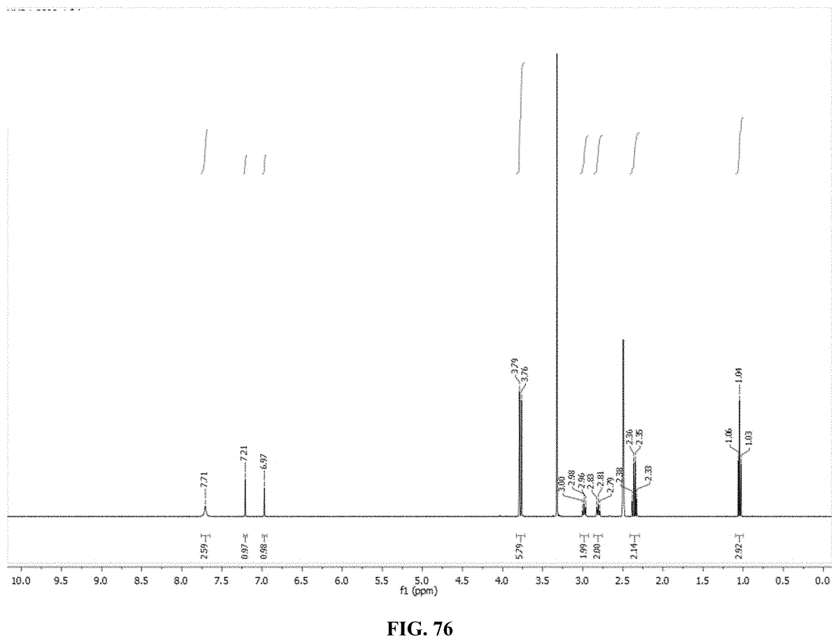

In some embodiments, 2C-B Esylate (Form 1) has an ¹H NMR spectra as provided in FIG. 76.

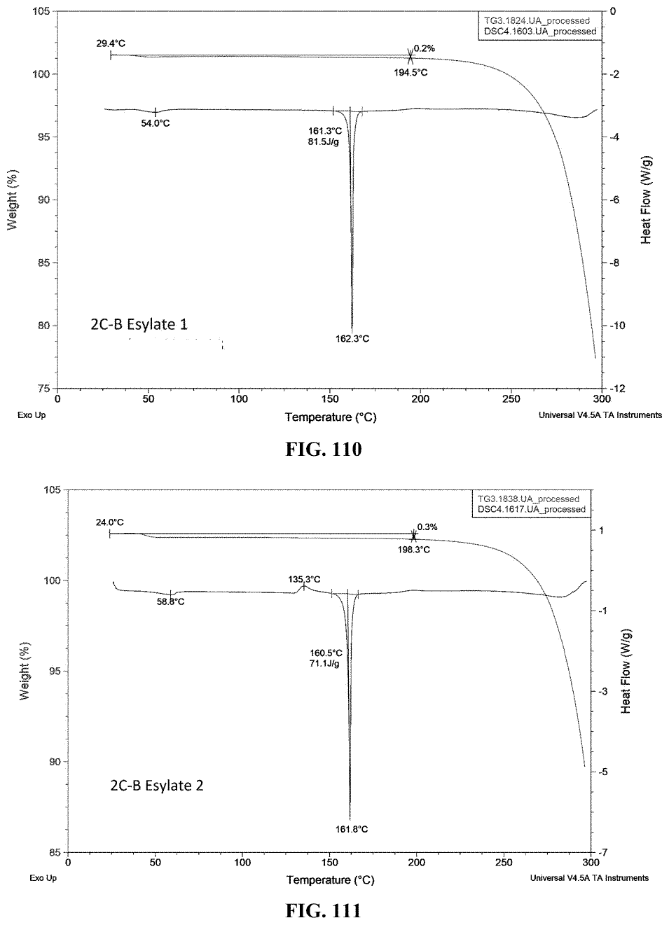

In some embodiments, 2C-B Esylate (Form 1) has TGA and DSC profiles as provided in FIG. 110.

In some embodiments, the 2C-B Esylate (Form 1) is a crystalline polymorph characterized by two or more, or three XRPD signals selected from the group consisting of 9.3 °2θ, 10.0 °2θ, and 14.5 °2θ (±0.2 °2θ; ±0.1 °2θ; or ±0.0 °2θ; Cu Kα1 radiation).

In some embodiments, the 2C-B Esylate (Form 1) is a crystalline polymorph characterized by two or more, or three XRPD signals selected from the group consisting of 9.3 °2θ, 10.0 °2θ, and 14.5 °2θ, 17.4 °2θ, and 18.7 °2θ (±0.2 °2θ; ±0.1 °2θ; or ±0.0 °2θ; Cu Kα1 radiation).

In some embodiments, the 2C-B Esylate is crystalline polymorphic (Form 2) characterized by two or more, or three XRPD signals selected from the group consisting of 24.1 °2θ, 13.9 °2θ, and 22.7 °2θ (±0.2 °2θ; ±0.1 °2θ; or ±0.0 °2θ; Cu Kα1 radiation).

In some embodiments, the 2C-B Esylate is crystalline polymorphic (Form 2) characterized by two or more, or three XRPD signals selected from the group consisting of 24.1 °2θ, 13.9 °2θ, 22.7 °2θ, and 15.2 °2θ (±0.2 °2θ; ±0.1 °2θ; or ±0.0 °2θ; Cu Kα1 radiation).

In some embodiments, the 2C-B Esylate is crystalline polymorphic (Form 2) characterized by two or more, or three XRPD signals selected from the group consisting of 24.1 °2θ, 13.9 °2θ, 22.7 °2θ, 15.2 °2θ, and 17.5 °2θ (±0.2 °2θ; ±0.1 °2θ; or ±0.0 °2θ; Cu Kα1 radiation).

In some embodiments, the 2C-B Esylate is crystalline polymorphic (Form 2) characterized by two or more, or three XRPD signals selected from the group consisting of 24.1 °2θ, 13.9 °2θ, 22.7 °2θ, 15.2 °2θ, 17.5 °2θ, 23.9 °2θ, 24.3 °2θ, 27 °2θ, 8.6 °2θ, and 12.5 °2θ (±0.2 °2θ; ±0.1 °2θ; or ±0.0 °2θ; Cu Kα1 radiation).

In some embodiments, the 2C-B Esylate is crystalline polymorphic (Form 2) characterized by two, three, four, five, six, seven, eight, nine, or ten XRPD signals as shown in Table 39.

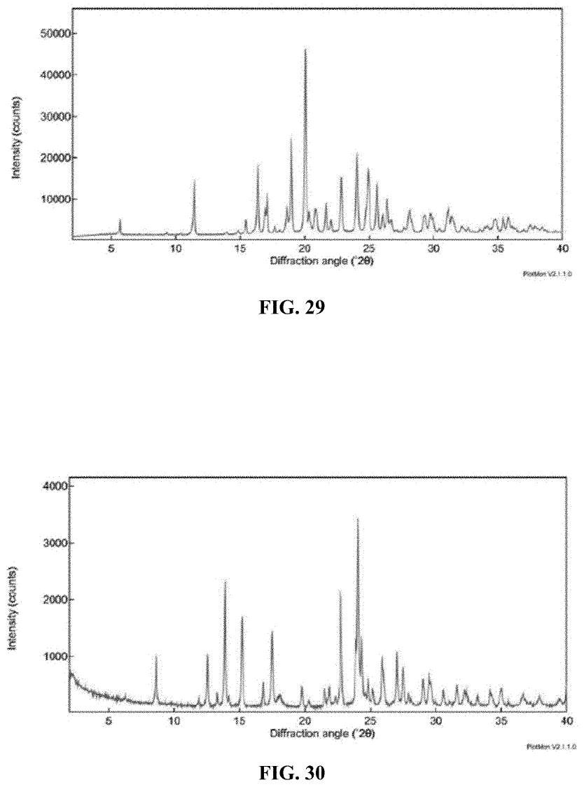

In some embodiments, the 2C-B Esylate is crystalline polymorphic (Form 2) characterized by two or more, or three XRPD signals as shown in FIG. 30.

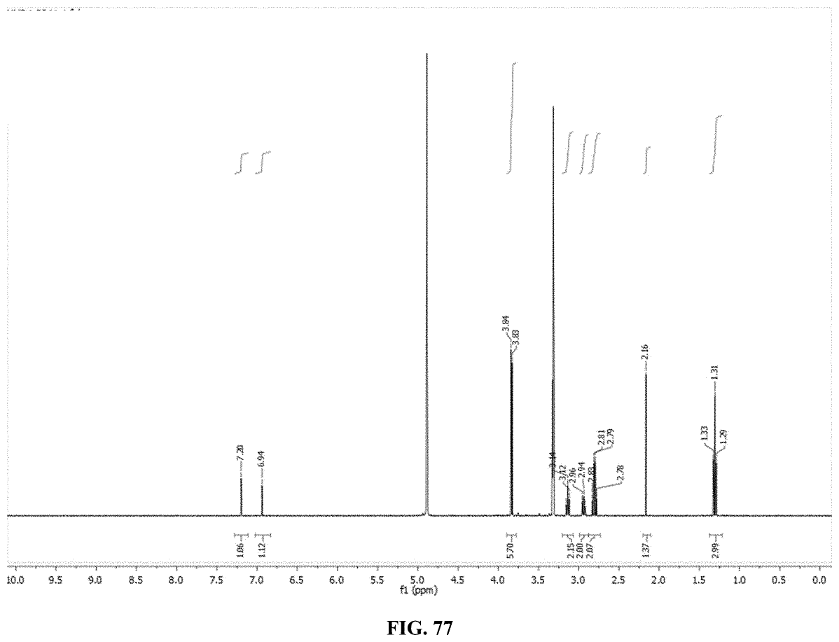

In some embodiments, 2C-B Esylate (Form 2) has an ¹H NMR spectra as provided in FIG. 77.

In some embodiments, 2C-B Esylate (Form 2) has TGA and DSC profiles as provided in FIG. 111.

In some embodiments, the 2C-B Esylate (Form 2) is a crystalline polymorph characterized by two or more, or three XRPD signals selected from the group consisting of 8.6 °2θ, 12.5 °2θ, and 13.9 °2θ (±0.2 °2θ; ±0.1 °2θ; or ±0.0 °2θ; Cu Kα1 radiation).

In some embodiments, the 2C-B Esylate (Form 2) is a crystalline polymorph characterized by two or more, or three XRPD signals selected from the group consisting of 8.6 °2θ, 12.5 °2θ, and 13.9 °2θ, 15.2 °2θ, and 17.5 °2θ (±0.2 °2θ; ±0.1 °2θ; or ±0.0 °2θ; Cu Kα1 radiation).

### 2C-B Fumarate Salt

In some embodiments, the 2C-B Fumarate salt is crystalline polymorphic (Form 1) characterized by two or more, or three XRPD signals selected from the group consisting of 22.9 °2θ, 14.6 °2θ, and 21.4 °2θ (±0.2 °2θ; ±0.1 °2θ; or ±0.0 °2θ; Cu Kα1 radiation).

In some embodiments, the 2C-B Fumarate salt is crystalline polymorphic (Form 1) characterized by two or more, or three XRPD signals selected from the group consisting of 22.9 °2θ, 14.6 °2θ, 21.4 °2θ, and 24.9 °2θ (±0.2 °2θ; ±0.1 °2θ; or ±0.0 °2θ; Cu Kα1 radiation).

In some embodiments, the 2C-B Fumarate salt is crystalline polymorphic (Form 1) characterized by two or more, or three XRPD signals selected from the group consisting of 22.9 °2θ, 14.6 °2θ, 21.4 °2θ, 24.9 °2θ, and 21.7 °2θ (±0.2 °2θ; ±0.1 °2θ; or ±0.0 °2θ; Cu Kα1 radiation).

In some embodiments, the 2C-B Fumarate salt is crystalline polymorphic (Form 1) characterized by two or more, or three XRPD signals selected from the group consisting of 22.9 °2θ, 14.6 °2θ, 21.4 °2θ, 24.9 °2θ, 21.7 °2θ, 25.3 °2θ, 7.1 °2θ, 24.5 °2θ, 28.9 °2θ, and 15.6 °2θ (±0.2 °2θ; ±0.1 °2θ; or ±0.0 °2θ; Cu Kα1 radiation).

In some embodiments, the 2C-B Fumarate salt is crystalline polymorphic (Form 1) characterized by two, three, four, five, six, seven, eight, nine, or ten XRPD signals as shown in Table 22.

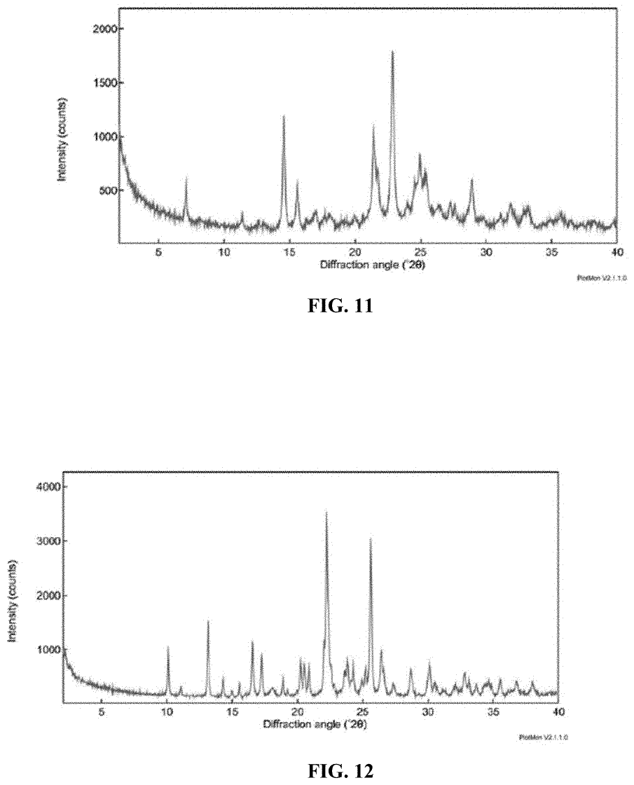

In some embodiments, the 2C-B Fumarate salt is crystalline polymorphic (Form 1) characterized by two or more, or three XRPD signals as shown in FIG. 11.

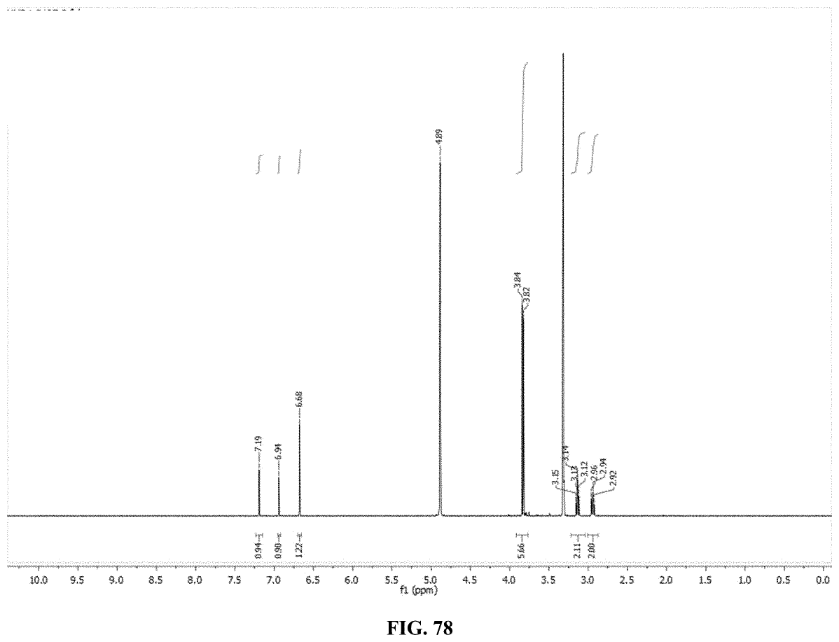

In some embodiments, 2C-B Fumarate (Form 1) has an ¹H NMR spectra as provided in FIG. 78.

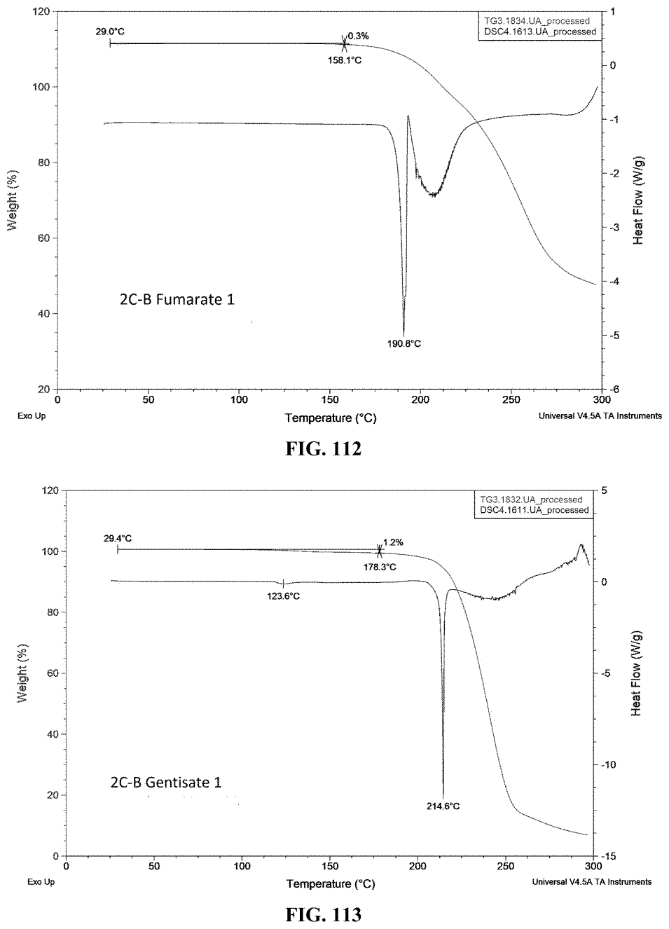

In some embodiments, 2C-B Fumarate (Form 1) has TGA and DSC profiles as provided in FIG. 112.

In some embodiments, the 2C-B Fumarate (Form 1) is a crystalline polymorph characterized by two or more, or three XRPD signals selected from the group consisting of 7.1 °2θ, 14.6 °2θ, and 15.6 °2θ (±0.2 °2θ; ±0.1 °2θ; or ±0.0 °2θ; Cu Kα1 radiation).

In some embodiments, the 2C-B Fumarate (Form 1) is a crystalline polymorph characterized by two or more, or three XRPD signals selected from the group consisting of 7.1 °2θ, 14.6 °2θ, 15.6 °2θ, 21.4 °2θ, and 22.9 °2θ (±0.2 °2θ; ±0.1 °2θ; or ±0.0 °2θ; Cu Kα1 radiation).

### 2C-B Gentisate Salt

In some embodiments, the 2C-B gentisate salt is a crystalline polymorph characterized by two or more, or three XRPD signals selected from the group consisting of 22.2 °2θ, 25.6 °2θ, 13.1 °2θ, 16.5 °2θ, and 10.1 °2θ (±0.2 °2θ; ±0.1 °2θ; or ±0.0 °2θ; Cu Kα1 radiation).

In some embodiments, the 2C-B gentisate salt is a crystalline polymorph characterized by two or more, or three XRPD signals selected from the group consisting of 22.2 °2θ, 25.6 °2θ, 13.1 °2θ, 16.5 °2θ, 10.1 °2θ, 26.4 °2θ, 17.3 °2θ, 23.8 °2θ, 20.2 °2θ, and 24.3 °2θ (±0.2 °2θ; ±0.1 °2θ; or ±0.0 °2θ; Cu Kα1 radiation).

In some embodiments, the 2C-B gentisate salt is a crystalline polymorph characterized by two or more, or three XRPD signals selected from the group consisting of 10.1 °2θ, 13.1 °2θ, and 16.5 °2θ (±0.2 °2θ; ±0.1 °2θ; or ±0.0 °2θ; Cu Kα1 radiation).

In some embodiments, the 2C-B gentisate salt is a crystalline polymorph characterized by two or more, or three XRPD signals selected from the group consisting of 10.1 °2θ, 13.1 °2θ, 16.5 °2θ, 17.3 °2θ, and 20.2 °2θ (±0.2 °2θ; ±0.1 °2θ; or ±0.0 °2θ; Cu Kα1 radiation).

In some embodiments, the 2C-B gentisate salt characterized by two, three, four, five, six, seven, eight, nine, or ten XRPD signals as shown in Table 23.

In some embodiments, the 2C-B gentisate salt characterized by two or more, or three XRPD signals as shown in FIG. 12.

In some embodiments, the 2C-B Gentisate is a crystalline polymorph characterized by two or more, or three XRPD signals selected from the group consisting of 10.1 °2θ, 13.1 °2θ, and 16.5 °2θ (±0.2 °2θ; ±0.1 °2θ; or ±0.0 °2θ; Cu Kα1 radiation).

In some embodiments, the 2C-B Gentisate is a crystalline polymorph characterized by two or more, or three XRPD signals selected from the group consisting of 10.1 °2θ, 13.1 °2θ, 16.5 °2θ, 17.2 °2θ, and 22.2 °2θ (±0.2 °2θ; ±0.1 °2θ; or ±0.0 °2θ; Cu Kα1 radiation).

In some embodiments, the 2C-B Gentisate salt is a crystalline polymorph characterized by two or more, or three XRPD signals selected from the group consisting of 22.2 °2θ, 25.6 °2θ, and 13.1 °2θ (±0.2 °2θ; ±0.1 °2θ; or ±0.0 °2θ; Cu Kα1 radiation).

In some embodiments, the 2C-B Gentisate salt is a crystalline polymorph characterized by two or more, or three XRPD signals selected from the group consisting of 22.2 °2θ, 25.6 °2θ, 13.1 °2θ, and 16.5 °2θ (±0.2 °2θ; ±0.1 °2θ; or ±0.0 °2θ; Cu Kα1 radiation).

In some embodiments, the 2C-B Gentisate salt is a crystalline polymorph characterized by two or more, or three XRPD signals selected from the group consisting of 22.2 °2θ, 25.6 °2θ, 13.1 °2θ, 16.5 °2θ, and 26.4 °2θ (±0.2 °2θ; ±0.1 °2θ; or ±0.0 °2θ; Cu Kα1 radiation).

In some embodiments, the 2C-B Gentisate salt is a crystalline polymorph characterized by two or more, or three XRPD signals selected from the group consisting of 22.2 °2θ, 25.6 °2θ, 13.1 °2θ, 16.5 °2θ, 26.4 °2θ, 17.2 °2θ, 23.8 °2θ, 20.5 °2θ, 24.3 °2θ, and 30.1 °2θ (±0.2 °2θ; ±0.1 °2θ; or ±0.0 °2θ; Cu Kα1 radiation).

In some embodiments, the 2C-B Gentisate salt is a crystalline polymorph characterized by two, three, four, five, six, seven, eight, nine, or ten XRPD signals as shown in Table 62.

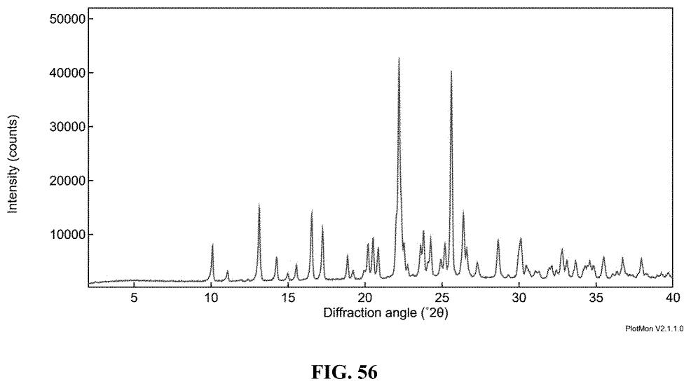

In some embodiments, the 2C-B Gentisate salt is a crystalline polymorph characterized by two or more, or three XRPD signals as shown in FIG. 56.

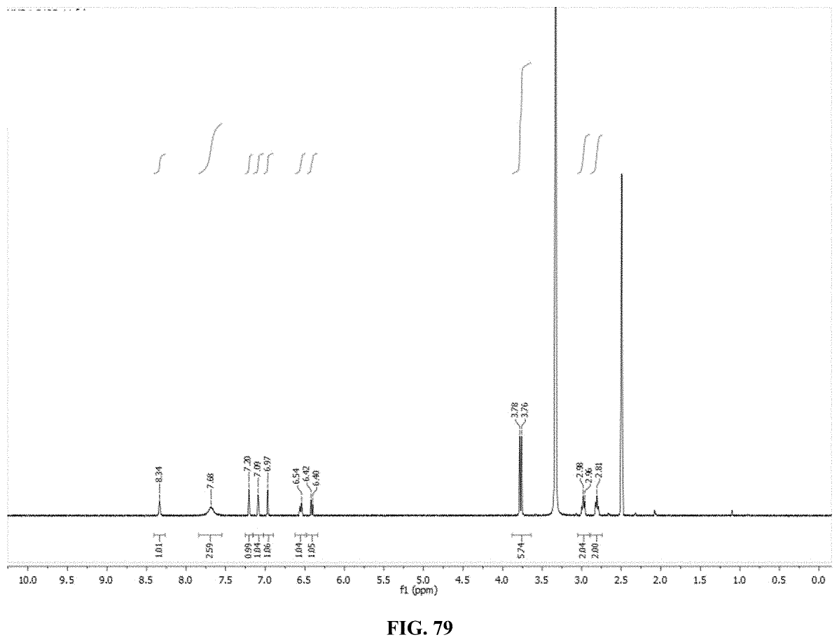

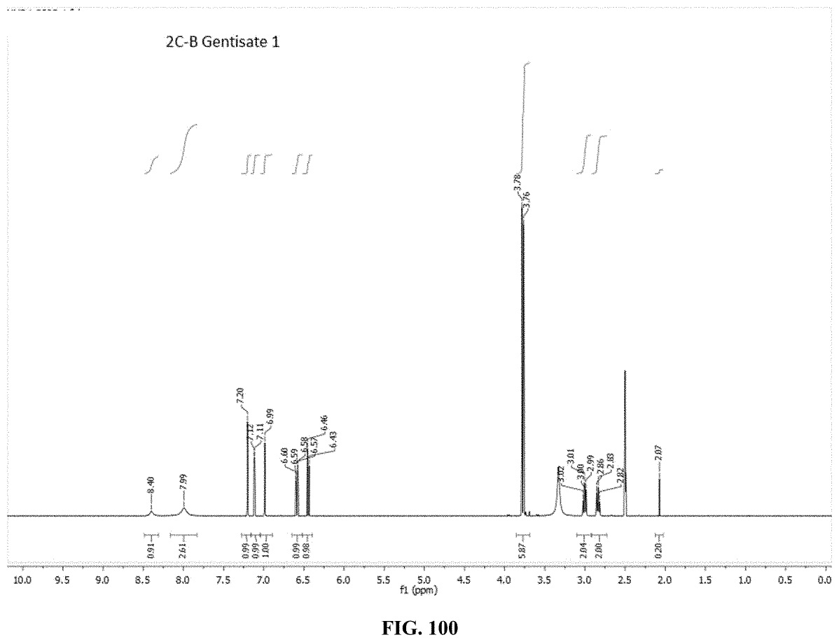

In some embodiments, 2C-B Gentisate has an ¹H NMR spectra as provided in FIG. 79. In some embodiments, 2C-B Gentisate has an ¹H NMR spectra as provided in FIG. 100.

In some embodiments, 2C-B Gentisate has TGA and DSC profiles as provided in FIG. 113.

In some embodiments, 2C-B Gentisate has TGA and DSC profiles as provided in FIG. 127.

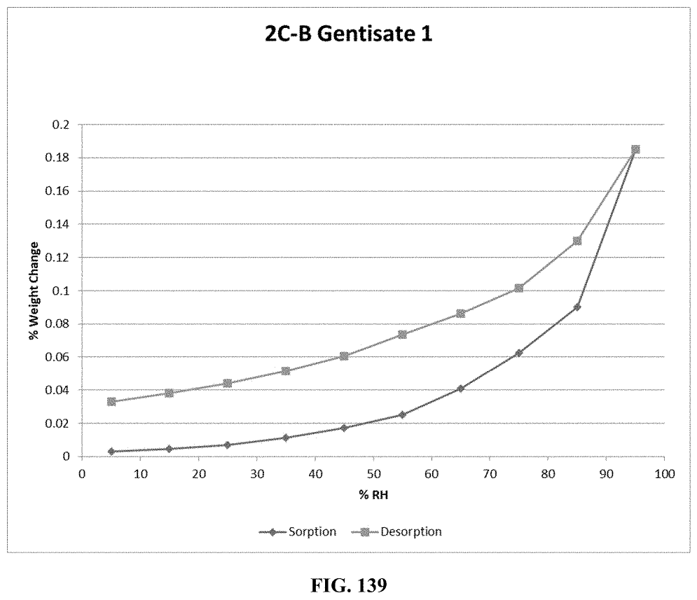

In some embodiments, 2C-B Gentisate has a DVS profile as provided in FIG. 139.

In some embodiments, the 2C-B Gentisate salt is crystalline polymorphic (PO) characterized by two or more, or three XRPD signals selected from the group consisting of 10.1 °2θ, 25.6 °2θ, and 22.2 °2θ (±0.2 °2θ; ±0.1 °2θ; or ±0.0 °2θ; Cu Kα1 radiation).

In some embodiments, the 2C-B Gentisate salt is crystalline polymorphic (P0) characterized by two or more, or three XRPD signals selected from the group consisting of 10.1 °2θ, 25.6 °2θ, 22.2 °2θ, and 20.2 °2θ (±0.2 °2θ; ±0.1 °2θ; or ±0.0 °2θ; Cu Kα1 radiation).

In some embodiments, the 2C-B Gentisate salt is crystalline polymorphic (P0) characterized by two or more, or three XRPD signals selected from the group consisting of 10.1 °2θ, 25.6 °2θ, 22.2 °2θ, 20.2 °2θ, and 13.1 °2θ (±0.2 °2θ; ±0.1 °2θ; or ±0.0 °2θ; Cu Kα1 radiation).

In some embodiments, the 2C-B Gentisate salt is crystalline polymorphic (P0) characterized by two or more, or three XRPD signals selected from the group consisting of 10.1 °2θ, 25.6 °2θ, 22.2 °2θ, 20.2 °2θ, 13.1 °2θ, 20.9 °2θ, 16.5 °2θ, 22 °2θ, 30.5 °2θ, and 17.2 °2θ (±0.2 °2θ; ±0.1 °2θ; or ±0.0 °2θ; Cu Kα1 radiation).

In some embodiments, the 2C-B Gentisate salt is crystalline polymorphic (P0) characterized by two, three, four, five, six, seven, eight, nine, or ten XRPD signals as shown in Table 63.

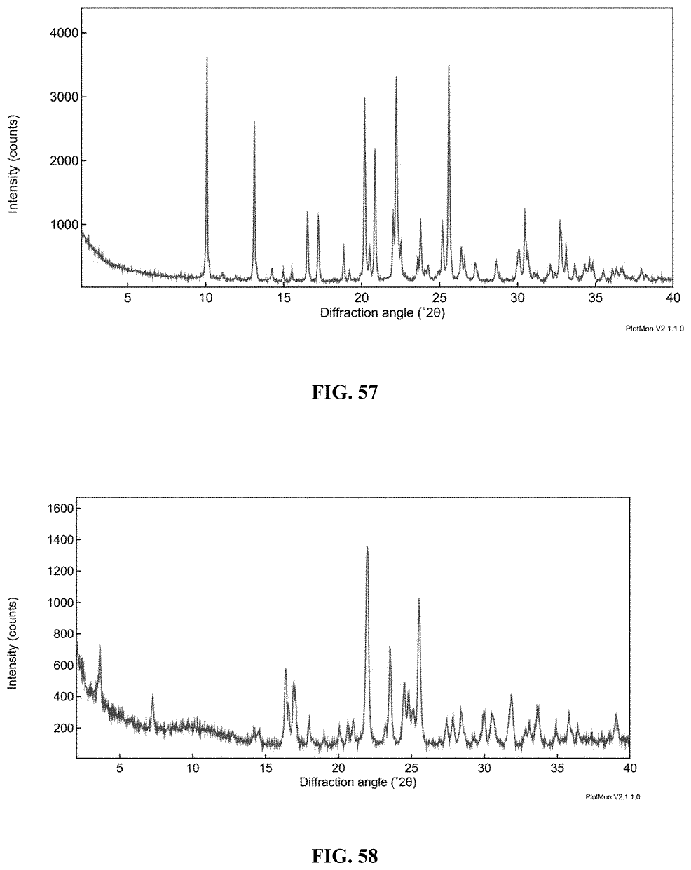

In some embodiments, the 2C-B Gentisate salt is crystalline polymorphic (P0) characterized by two or more, or three XRPD signals as shown in FIG. 57.

In some embodiments, the 2C-B gentisate salt possess at least one of the following characteristics:

(a) exhibit high crystallinity,

(b) exhibit acceptable scale-up

(c) exhibit minimal water uptake over RH range,

(d) exhibit uncomplicated DSC data (one endothermic event observed), and

(e) acceptable solubility when compared to the other salts.

In some embodiments, the 2C-B gentisate salt possess at least one of characteristic as provided in Table 86.

### 2C-B Glycolate Salt

In some embodiments, the 2C-B Glycolate salt is a crystalline polymorph characterized by two or more, or three XRPD signals selected from the group consisting of 26.4 °2θ, 19 °2θ, and 21.5 °2θ (±0.2 °2θ; ±0.1 °2θ; or ±0.0 °2θ; Cu Kα1 radiation).

In some embodiments, the 2C-B Glycolate salt is a crystalline polymorph characterized by two or more, or three XRPD signals selected from the group consisting of 26.4 °2θ, 19 °2θ, 21.5 °2θ, and 25.4 °2θ (±0.2 °2θ; ±0.1 °2θ; or ±0.0 °2θ; Cu Kα1 radiation).

In some embodiments, the 2C-B Glycolate salt is a crystalline polymorph characterized by two or more, or three XRPD signals selected from the group consisting of 26.4 °2θ, 19 °2θ, 21.5 °2θ, 25.4 °2θ, and 22.9 °2θ (±0.2 °2θ; ±0.1 °2θ; or ±0.0 °2θ; Cu Kα1 radiation).

In some embodiments, the 2C-B Glycolate salt is a crystalline polymorph characterized by two or more, or three XRPD signals selected from the group consisting of 26.4 °2θ, 19 °2θ, 21.5 °2θ, 25.4 °2θ, 22.9 °2θ, 15.6 °2θ, 16 °2θ, 27 °2θ, 27.2 °2θ, and 6.3 °2θ (±0.2 °2θ; ±0.1 °2θ; or ±0.0 °2θ; Cu Kα1 radiation).

In some embodiments, the 2C-B Glycolate salt is a crystalline polymorph characterized by two or more, or three XRPD signals selected from the group consisting of 6.3 °2θ, 15.6 °2θ, and 16 °2θ (±0.2 °2θ; ±0.1 °2θ; or ±0.0 °2θ; Cu Kα1 radiation).

In some embodiments, the 2C-B Glycolate salt is a crystalline polymorph characterized by two or more, or three XRPD signals selected from the group consisting of 6.3 °2θ, 15.6 °2θ, 16 °2θ, 19 °2θ, and 21.5 °2θ (±0.2 °2θ; ±0.1 °2θ; or ±0.0 °2θ; Cu Kα1 radiation).

In some embodiments, the 2C-B Glycolate salt is a crystalline polymorph characterized by two, three, four, five, six, seven, eight, nine, or ten XRPD signals as shown in Table 25.

In some embodiments, the 2C-B Glycolate salt is crystalline polymorphic characterized by two or more, or three XRPD signals as shown in FIG. 14.

In some embodiments, the 2C-B Glycolate is a crystalline polymorph characterized by two or more, or three XRPD signals selected from the group consisting of 6.4 °2θ, 15.7 °2θ, and 16.1 °2θ (±0.2 °2θ; ±0.1 °2θ; or ±0.0 °2θ; Cu Kα1 radiation).

In some embodiments, the 2C-B Glycolate is a crystalline polymorph characterized by two or more, or three XRPD signals selected from the group consisting of 6.4 °2θ, 15.7 °2θ, 16.1 °2θ, 19.0 °2θ, and 21.5 °2θ (±0.2 °2θ; ±0.1 °2θ; or ±0.0 °2θ; Cu Kα1 radiation).

In some embodiments, the 2C-B Glycolate salt is a crystalline polymorph characterized by two or more, or three XRPD signals selected from the group consisting of 26.3 °2θ, 21.5 °2θ, and 19 °2θ (±0.2 °2θ; ±0.1 °2θ; or ±0.0 °2θ; Cu Kα1 radiation).

In some embodiments, the 2C-B Glycolate salt is a crystalline polymorph characterized by two or more, or three XRPD signals selected from the group consisting of 26.3 °2θ, 21.5 °2θ, 19 °2θ, and 16.1 °2θ (±0.2 °2θ; ±0.1 °2θ; or ±0.0 °2θ; Cu Kα1 radiation).

In some embodiments, the 2C-B Glycolate salt is a crystalline polymorph characterized by two or more, or three XRPD signals selected from the group consisting of 26.3 °2θ, 21.5 °2θ, 19 °2θ, 16.1 °2θ, and 15.7 °2θ (±0.2 °2θ; ±0.1 °2θ; or ±0.0 °2θ; Cu Kα1 radiation).

In some embodiments, the 2C-B Glycolate salt is a crystalline polymorph characterized by two or more, or three XRPD signals selected from the group consisting of 26.3 °2θ, 21.5 °2θ, 19 °2θ, 16.1 °2θ, 15.7 °2θ, 23 °2θ, 25.5 °2θ, 27 °2θ, 27.2 °2θ, and 25.7 °2θ (±0.2 °2θ; ±0.1 °2θ; or ±0.0 °2θ; Cu Kα1 radiation).

In some embodiments, the 2C-B Glycolate salt is a crystalline polymorph characterized by two, three, four, five, six, seven, eight, nine, or ten XRPD signals as shown in Table 65.

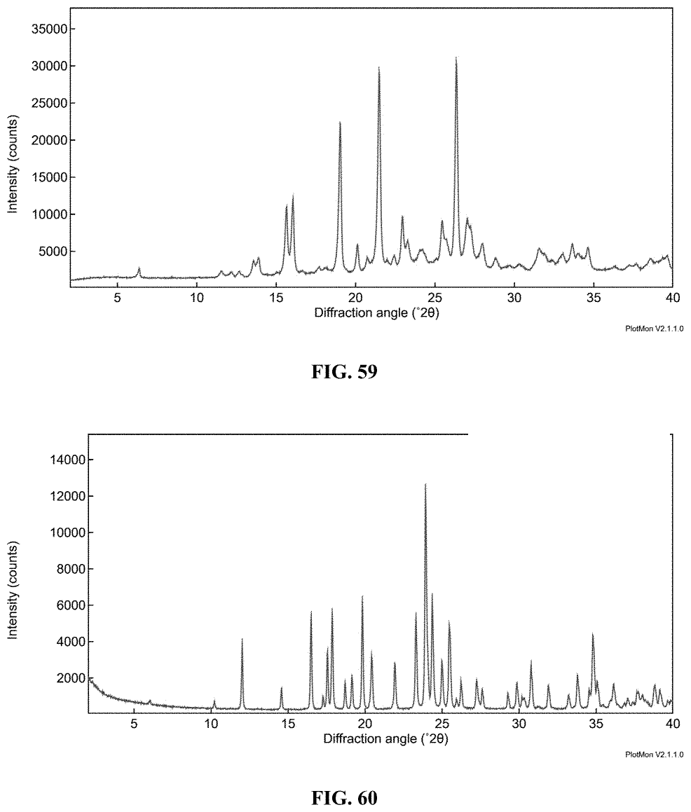

In some embodiments, the 2C-B Glycolate salt is crystalline polymorphic characterized by two or more, or three XRPD signals as shown in FIG. 59.

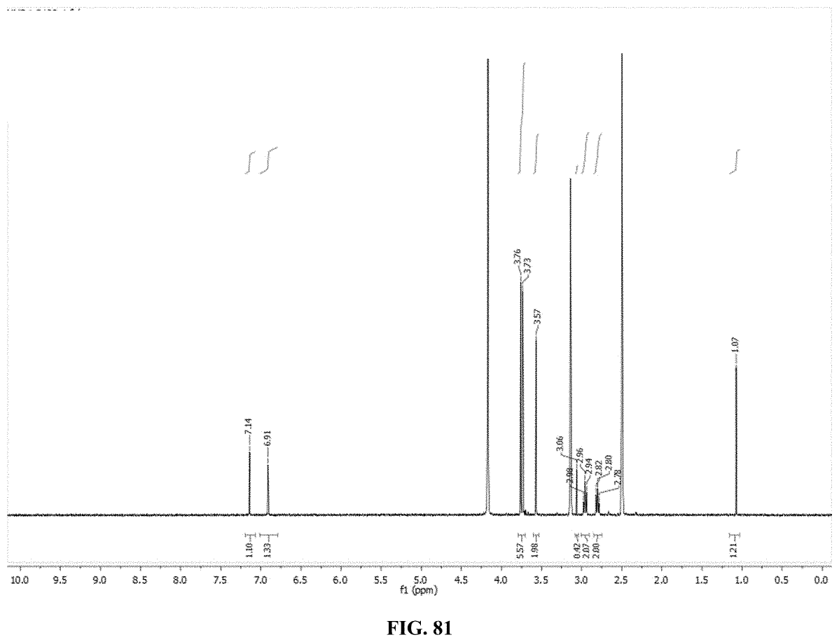

In some embodiments, 2C-B Glycolate has an ¹H NMR spectra as provided in FIG. 81.

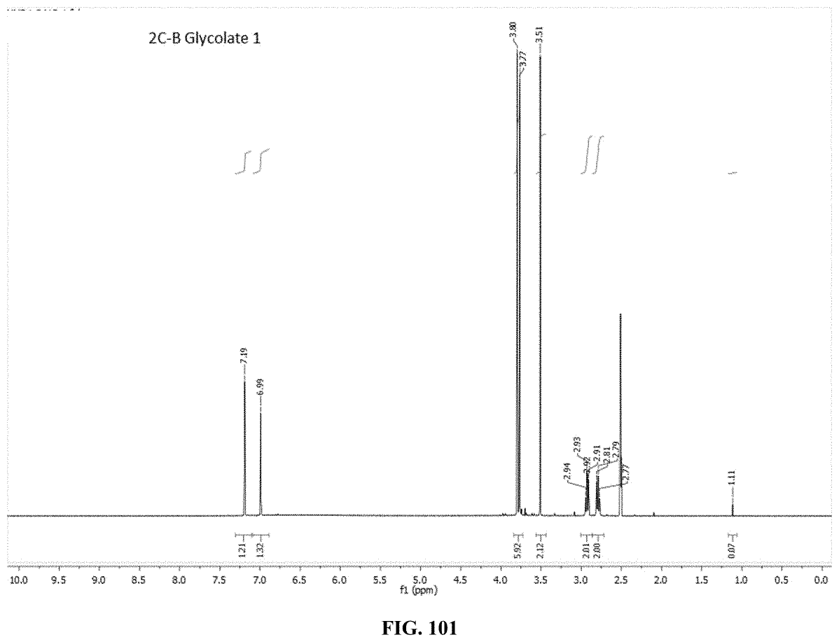

In some embodiments, 2C-B Glycolate has an ¹H NMR spectra as provided in FIG. 101.

In some embodiments, 2C-B Glycolate has TGA and DSC profiles as provided in FIG. 115.

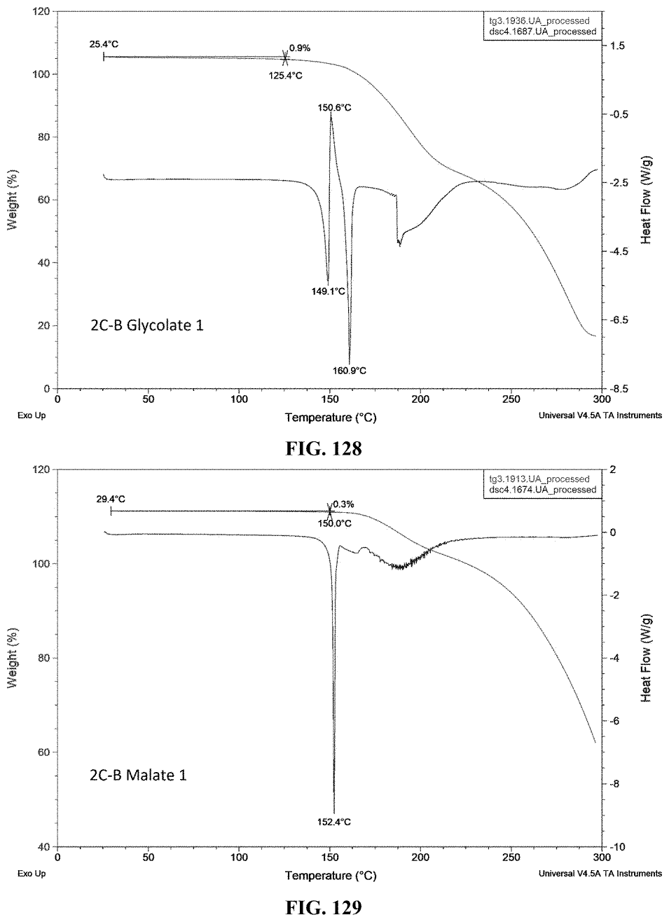

In some embodiments, 2C-B Glycolate has TGA and DSC profiles as provided in FIG. 128.

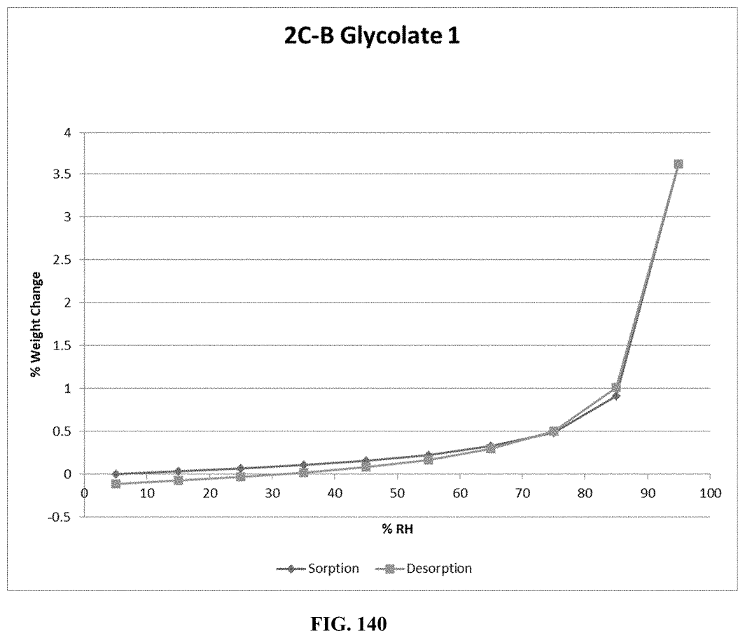

In some embodiments, 2C-B Glycolate has a DVS profile as provided in FIG. 140.

In some embodiments, the 2C-B glycolate salt possess at least one of the following characteristics:

(a) exhibit high crystallinity,

(b) exhibit acceptable scale-up

(c) exhibit minimal water uptake over RH range,

(d) exhibit uncomplicated DSC data (one endothermic event observed), and

(e) acceptable solubility when compared to the other salts.

In some embodiments, the 2C-B glycolate salt possess at least one of characteristic as provided in Table 86.

### 2C-B Sulfate Salt

In some embodiments, the 2C-B sulfate salt is crystalline polymorphic characterized by two or more, or three XRPD signals selected from the group consisting of 25.1 °2θ, 25.7 °2θ, and 23.2 °2θ (±0.2 °2θ; ±0.1 °2θ; or ±0.0 °2θ; Cu Kα1 radiation).

In some embodiments, the 2C-B sulfate salt is a crystalline polymorph characterized by two or more, or three XRPD signals selected from the group consisting of 25.1 °2θ, 25.7 °2θ, 23.2 °2θ, and 8.7 °2θ (±0.2 °2θ; ±0.1 °2θ; or ±0.0 °2θ; Cu Kα1 radiation).

In some embodiments, the 2C-B sulfate salt is a crystalline polymorph characterized by two or more, or three XRPD signals selected from the group consisting of 25.1 °2θ, 25.7 °2θ, 23.2 °2θ, 8.7 °2θ, and 13 °2θ (±0.2 °2θ; ±0.1 °2θ; or ±0.0 °2θ; Cu Kα1 radiation).

In some embodiments, the 2C-B sulfate salt is a crystalline polymorph characterized by two or more, or three XRPD signals selected from the group consisting of 25.1 °2θ, 25.7 °2θ, 23.2 °2θ, 8.7 °2θ, 13 °2θ, 21.8 °2θ, 28.5 °2θ, 18 °2θ, 20.4 °2θ, and 27.8 °2θ (±0.2 °2θ; ±0.1 °2θ; or ±0.0 °2θ; Cu Kα1 radiation).

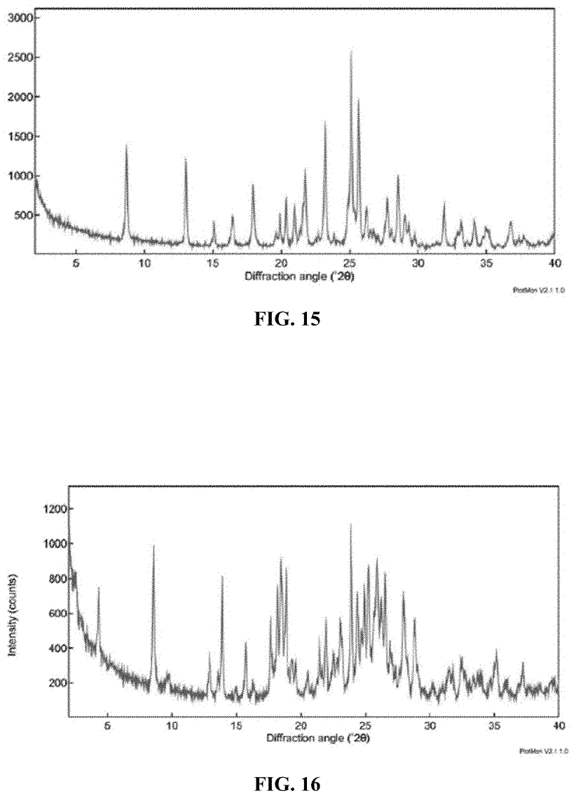

In some embodiments, the 2C-B sulfate salt is a crystalline polymorph characterized by two, three, four, five, six, seven, eight, nine, or ten XRPD signals as shown in Table 26. In some embodiments, the 2C-B sulfate salt is a crystalline polymorph characterized by two or more, or three XRPD signals as shown in FIG. 15.

In some embodiments, the 2C-B Sulfate is a crystalline polymorph characterized by two or more, or three XRPD signals selected from the group consisting of 8.7 °2θ, 13.0 °2θ, and 16.4 °2θ (±0.2 °2θ; ±0.1 °2θ; or ±0.0 °2θ; Cu Kα1 radiation). In some embodiments, the 2C-B Sulfate is a crystalline polymorph characterized by two or more, or three XRPD signals selected from the group consisting of 8.7 °2θ, 13.0 °2θ, 16.4 °2θ, 15.1 °2θ and 17.9 °2θ (±0.2 °2θ; ±0.1 °2θ; or ±0.0 °2θ; Cu Kα1 radiation).

In some embodiments, the 2C-B Sulfate is a crystalline polymorph characterized by two or more, or three XRPD signals selected from the group consisting of 8.7 °2θ, 13.0 °2θ, 16.4 °2θ, 15.1 °2θ and 18 °2θ (±0.2 °2θ; ±0.1 °2θ; or ±0.0 °2θ; Cu Kα1 radiation).

In some embodiments, the 2C-B Sulfate salt is a crystalline polymorph characterized by two or more, or three XRPD signals selected from the group consisting of 25.1 °2θ, 8.7 °2θ, and 23.2 °2θ (±0.2 °2θ; ±0.1 °2θ; or ±0.0 °2θ; Cu Kα1 radiation).

In some embodiments, the 2C-B Sulfate salt is crystalline polymorphic characterized by two or more, or three XRPD signals selected from the group consisting of 25.1 °2θ, 8.7 °2θ, 23.2 °2θ, and 25.6 °2θ (±0.2 °2θ; ±0.1 °2θ; or ±0.0 °2θ; Cu Kα1 radiation).

In some embodiments, the 2C-B Sulfate salt is a crystalline polymorph characterized by two or more, or three XRPD signals selected from the group consisting of 25.1 °2θ, 8.7 °2θ, 23.2 °2θ, 25.6 °2θ, and 13 °2θ (±0.2 °2θ; ±0.1 °2θ; or ±0.0 °2θ; Cu Kα1 radiation). In some embodiments, the 2C-B Sulfate salt is a crystalline polymorph characterized by two or more, or three XRPD signals selected from the group consisting of 25.1 °2θ, 8.7 °2θ, 23.2 °2θ, 25.6 °2θ, 13 °2θ, 21.8 °2θ, 17.9 °2θ, 28.5 °2θ, 16.4 °2θ, and 27.8 °2θ (±0.2 °2θ; ±0.1 °2θ; or ±0.0 °2θ; Cu Kα1 radiation).

In some embodiments, the 2C-B Sulfate salt is a crystalline polymorph characterized by two, three, four, five, six, seven, eight, nine, or ten XRPD signals as shown in Table 68.

In some embodiments, the 2C-B Sulfate salt is a crystalline polymorph characterized by two or more, or three XRPD signals as shown in FIG. 62.

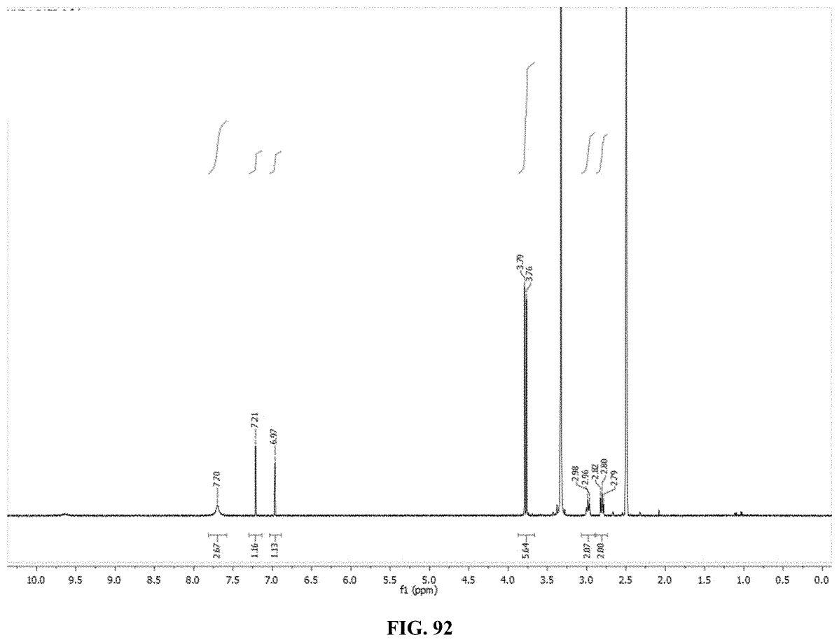

In some embodiments, 2C-B Sulfate has an ¹H NMR spectra as provided in FIG. 92.

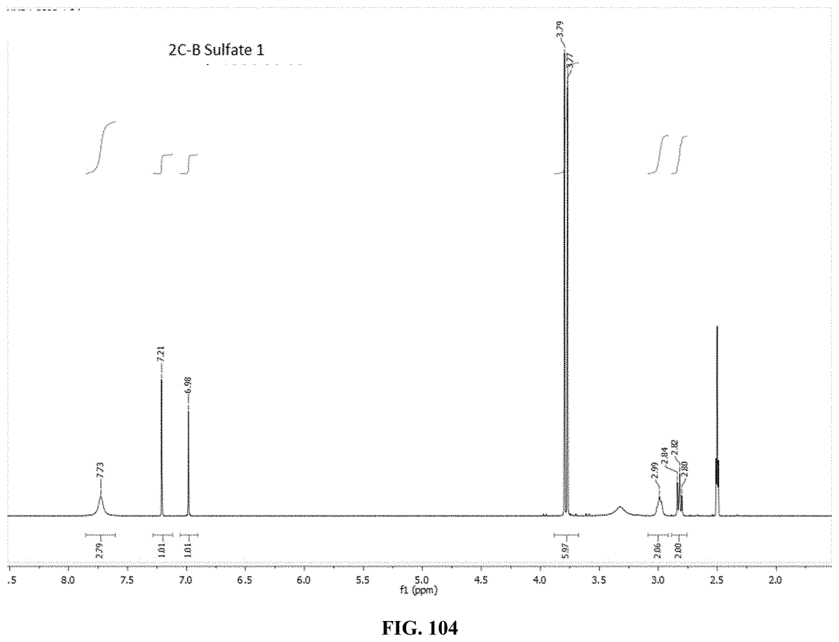

In some embodiments, 2C-B Sulfate has an ¹H NMR spectra as provided in FIG. 104.

In some embodiments, 2C-B Sulfate has TGA and DSC profiles as provided in FIG. 121.

In some embodiments, 2C-B Sulfate has TGA and DSC profiles as provided in FIG. 131.

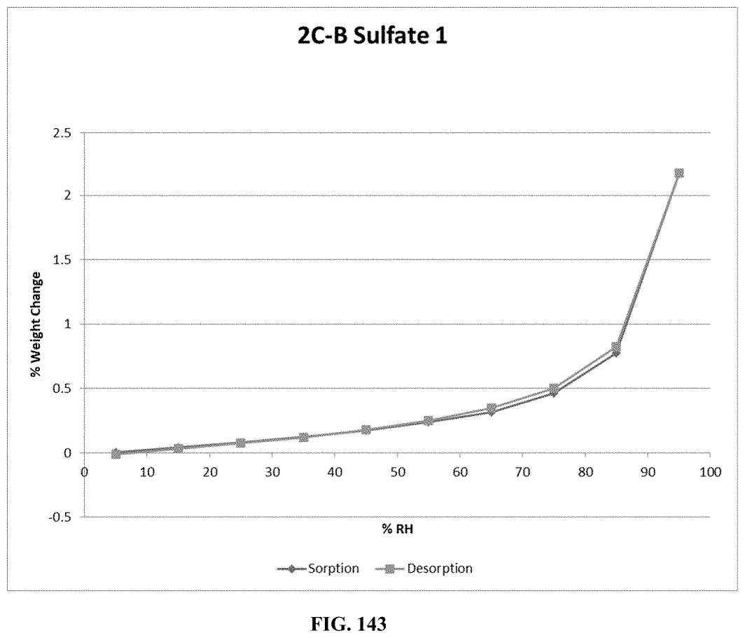

In some embodiments, 2C-B Sulfate has a DVS profile as provided in FIG. 143.

In some embodiments, the 2C-B sulfate salt possesses at least one of the following characteristics:

(a) exhibit high crystallinity,

(b) exhibit acceptable scale-up

(c) exhibit minimal water uptake over RH range,

(d) exhibit uncomplicated DSC data (one endothermic event observed), and

(e) acceptable solubility when compared to the other salts.

In some embodiments, the 2C-B sulfate salt possesses at least one of characteristic as provided in Table 86.

### 2C-B Phosphate Salt

In some embodiments, the 2C-B Phosphate salt is crystalline polymorphic (Form 1 and Form 2) characterized by two or more, or three XRPD signals selected from the group consisting of 23.9 °2θ, 18.5 °2θ, and 25.3 °2θ (±0.2 °2θ; ±0.1 °2θ; or ±0.0 °2θ; Cu Kα1 radiation).

In some embodiments, the 2C-B Phosphate salt is crystalline polymorphic (Form 1 and Form 2) characterized by two or more, or three XRPD signals selected from the group consisting of 23.9 °2θ, 18.5 °2θ, 25.3 °2θ, and 18.8 °2θ (±0.2 °2θ; ±0.1 °2θ; or ±0.0 °2θ; Cu Kα1 radiation).

In some embodiments, the 2C-B Phosphate salt is crystalline polymorphic (Form 1 and Form 2) characterized by two or more, or three XRPD signals selected from the group consisting of 23.9 °2θ, 18.5 °2θ, 25.3 °2θ, 18.8 °2θ, and 26.5 °2θ (±0.2 °2θ; ±0.1 °2θ; or ±0.0 °2θ; Cu Kα1 radiation).

In some embodiments, the 2C-B Phosphate salt is crystalline polymorphic (Form 1 and Form 2) characterized by two or more, or three XRPD signals selected from the group consisting of 23.9 °2θ, 18.5 °2θ, 25.3 °2θ, 18.8 °2θ, 26.5 °2θ, 13.9 °2θ, 24.9 °2θ, 18.2 °2θ, 4.3 °2θ, and 24.4 °2θ (±0.2 °2θ; ±0.1 °2θ; or ±0.0 °2θ; Cu Kα1 radiation).

In some embodiments, the 2C-B Phosphate salt is crystalline polymorphic (Form 1 and Form 2) characterized by two, three, four, five, six, seven, eight, nine, or ten XRPD signals as shown in Table 27. In some embodiments, the 2C-B Phosphate salt is crystalline polymorphic (Form 1 and Form 2) characterized by two or more, or three XRPD signals as shown in FIG. 16.

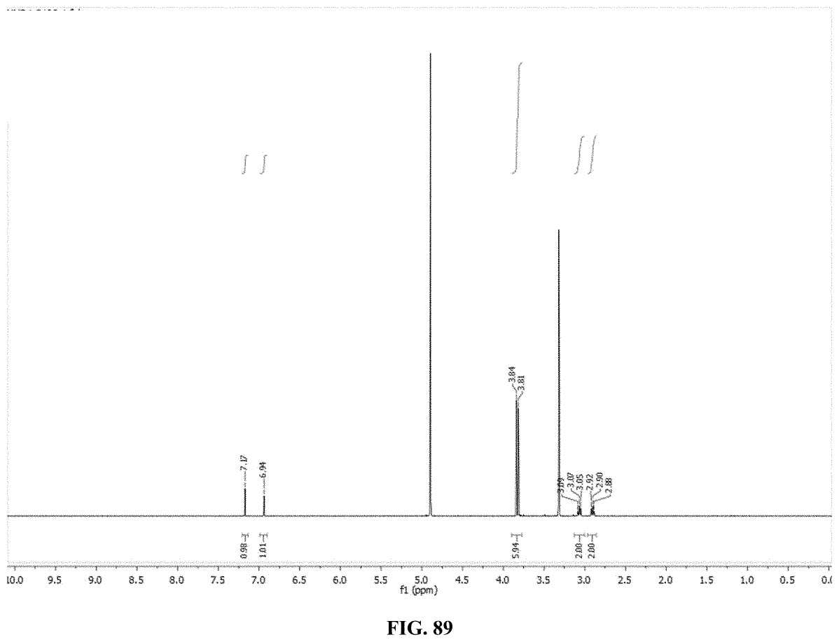

In some embodiments, 2C-B Phosphate (Form 1) has an ¹H NMR spectra as provided in FIG. 89.

In some embodiments, 2C-B Phosphate (Form 1) has TGA and DSC profiles as provided in FIG. 119.

In some embodiments, the 2C-B Phosphate (Form 1 and Form 2) is a crystalline polymorph characterized by two or more, or three XRPD signals selected from the group consisting of 4.3 °2θ, 13.9 °2θ, and 15.7 °2θ (±0.2 °2θ; ±0.1 °2θ; or ±0.0 °2θ; Cu Kα1 radiation).

In some embodiments, the 2C-B Phosphate (Form 1 and Form 2) is a crystalline polymorph characterized by two or more, or three XRPD signals selected from the group consisting of 4.3 °2θ, 13.9 °2θ, 15.7 °2θ, 18.5 °2θ, and 18.8 °2θ (±0.2 °2θ; ±0.1 °2θ; or ±0.0 °2θ; Cu Kα1 radiation).

In some embodiments, the 2C-B Phosphate salt is crystalline polymorphic (Form 2) characterized by two or more, or three XRPD signals selected from the group consisting of 24.8 °2θ, 8.6 °2θ, and 21.5 °2θ (±0.2 °2θ; ±0.1 °2θ; or ±0.0 °2θ; Cu Kα1 radiation).

In some embodiments, the 2C-B Phosphate salt is crystalline polymorphic (Form 2) characterized by two or more, or three XRPD signals selected from the group consisting of 24.8 °2θ, 8.6 °2θ, 21.5 °2θ, and 28.7 °2θ (±0.2 °2θ; ±0.1 °2θ; or ±0.0 °2θ; Cu Kα1 radiation).

In some embodiments, the 2C-B Phosphate salt is crystalline polymorphic (Form 2) characterized by two or more, or three XRPD signals selected from the group consisting of 24.8 °2θ, 8.6 °2θ, 21.5 °2θ, 28.7 °2θ, and 12.9 °2θ (±0.2 °2θ; ±0.1 °2θ; or ±0.0 °2θ; Cu Kα1 radiation).

In some embodiments, the 2C-B Phosphate salt is crystalline polymorphic (Form 2) characterized by two or more, or three XRPD signals selected from the group consisting of 24.8 °2θ, 8.6 °2θ, 21.5 °2θ, 28.7 °2θ, 12.9 °2θ, 25.9 °2θ, 39.3 °2θ, 32.8 °2θ, 22.9 °2θ, and 34.8 °2θ (±0.2 °2θ; ±0.1 °2θ; or ±0.0 °2θ; Cu Kα1 radiation).

In some embodiments, the 2C-B Phosphate salt is crystalline polymorphic (Form 2) characterized by two, three, four, five, six, seven, eight, nine, or ten XRPD signals as shown in Table 40.

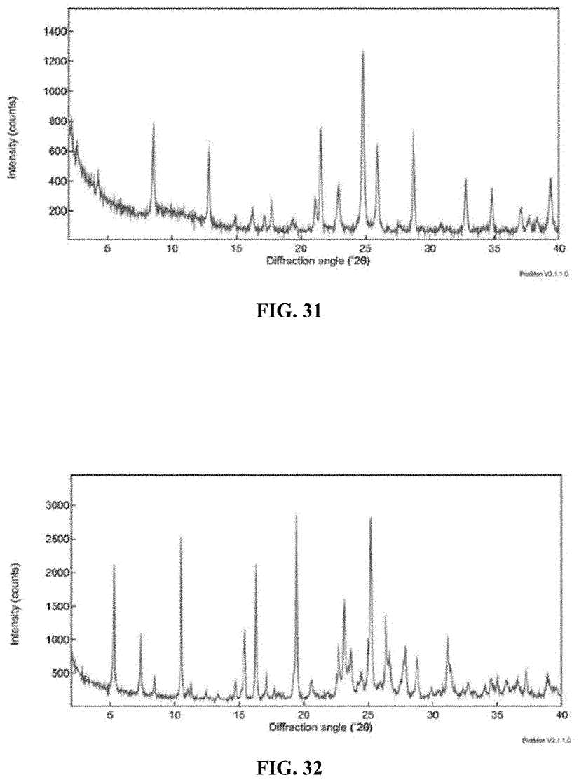

In some embodiments, the 2C-B Phosphate salt is crystalline polymorphic (Form 2) characterized by two or more, or three XRPD signals as shown in FIG. 31.

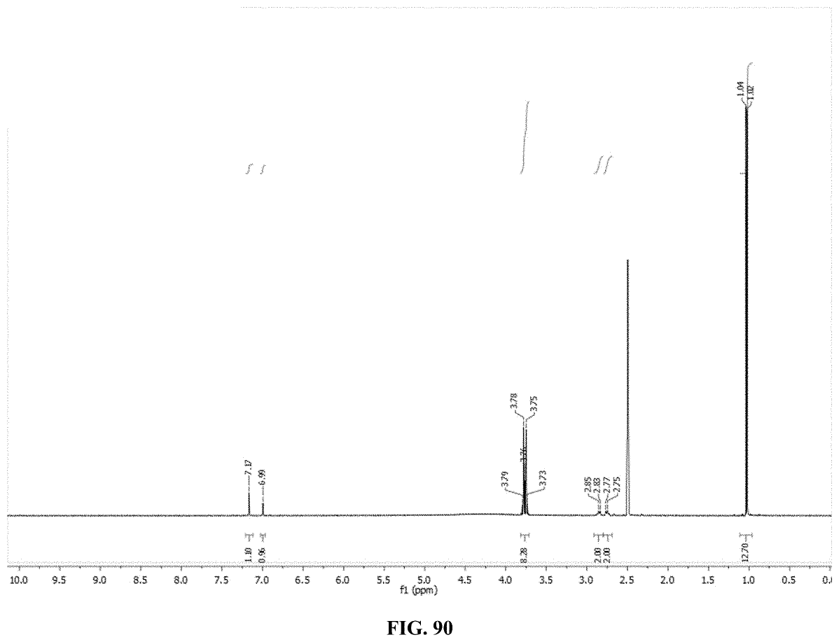

In some embodiments, 2C-B Phosphate (Form 2) has an ¹H NMR spectra as provided in FIG. 90.

In some embodiments, the 2C-B Phosphate (Form 2) is a crystalline polymorph characterized by two or more, or three XRPD signals selected from the group consisting of 8.6 °2θ, 12.9 °2θ, and 17.7 °2θ (±0.2 °2θ; ±0.1 °2θ; or ±0.0 °2θ; Cu Kα1 radiation).

In some embodiments, the 2C-B Phosphate (Form 2) is a crystalline polymorph characterized by two or more, or three XRPD signals selected from the group consisting of 8.6 °2θ, 12.9 °2θ, and 17.7 °2θ, 21.5 °2θ, and 24.8 °2θ (±0.2 °2θ; ±0.1 °2θ; or ±0.0 °2θ; Cu Kα1 radiation).

### 2C-B Xinafoate Salt

In some embodiments, the 2C-B Xinafoate is a crystalline polymorph characterized by two or more, or three XRPD signals selected from the group consisting of 24.7 °2θ, 23.3 °2θ, and 18.5 °2θ (±0.2 °2θ; ±0.1 °2θ; or ±0.0 °2θ; Cu Kα1 radiation).

In some embodiments, the 2C-B Xinafoate is a crystalline polymorph characterized by two or more, or three XRPD signals selected from the group consisting of 24.7 °2θ, 23.3 °2θ, 18.5 °2θ, and 26.1 °2θ (±0.2 °2θ; ±0.1 °2θ; or ±0.0 °2θ; Cu Kα1 radiation).

In some embodiments, the 2C-B Xinafoate is a crystalline polymorph characterized by two or more, or three XRPD signals selected from the group consisting of 24.7 °2θ, 23.3 °2θ, 18.5 °2θ, 26.1 °2θ, and 7.9 °2θ (±0.2 °2θ; ±0.1 °2θ; or ±0.0 °2θ; Cu Kα1 radiation).

In some embodiments, the 2C-B Xinafoate is a crystalline polymorph characterized by two or more, or three XRPD signals selected from the group consisting of 24.7 °2θ, 23.3 °2θ, 18.5 °2θ, 26.1 °2θ, 7.9 °2θ, 20 °2θ, 22.4 °2θ, 14.3 °2θ, 13.8 °2θ, and 16.4 °2θ (±0.2 °2θ; ±0.1 °2θ; or ±0.0 °2θ; Cu Kα1 radiation).

In some embodiments, the 2C-B Xinafoate is a crystalline polymorph characterized by two or more, or three XRPD signals selected from the group consisting of 7.9 °2θ, 13.8 °2θ, and 14.3 °2θ (±0.2 °2θ; ±0.1 °2θ; or ±0.0 °2θ; Cu Kα1 radiation).

In some embodiments, the 2C-B Xinafoate is a crystalline polymorph characterized by two or more, or three XRPD signals selected from the group consisting of 7.9 °2θ, 13.8 °2θ, 14.3 °2θ, 13.0 °2θ, and 18.5 °2θ (±0.2 °2θ; ±0.1 °2θ; or ±0.0 °2θ; Cu Kα1 radiation).

In some embodiments, the 2C-B Xinafoate is a crystalline polymorph characterized by two, three, four, five, six, seven, eight, nine, or ten XRPD signals as shown in Table 28.

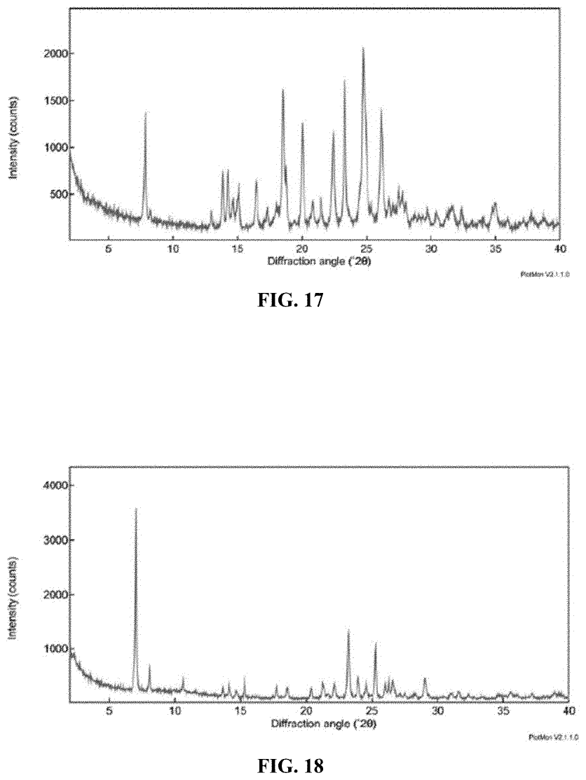

In some embodiments, the 2C-B Xinafoate is a crystalline polymorph characterized by two or more, or three XRPD signals as shown in FIG. 17.

In some embodiments, the 2C-B Xinafoate is a crystalline polymorph characterized by two or more, or three XRPD signals selected from the group consisting of 7.9 °2θ, 13.9 °2θ, and 14.4 °2θ (±0.2 °2θ; ±0.1 °2θ; or ±0.0 °2θ; Cu Kα1 radiation).

In some embodiments, the 2C-B Xinafoate is a crystalline polymorph characterized by two or more, or three XRPD signals selected from the group consisting of 7.9 °2θ, 13.9 °2θ, 14.4 °2θ, 13.1 °2θ, and 18.6 °2θ (±0.2 °2θ; ±0.1 °2θ; or ±0.0 °2θ; Cu Kα1 radiation).

In some embodiments, the 2C-B Xinafoate salt is a crystalline polymorph characterized by two or more, or three XRPD signals selected from the group consisting of 24.8 °2θ, 23.3 °2θ, and 18.6 °2θ (±0.2 °2θ; ±0.1 °2θ; or ±0.0 °2θ; Cu Kα1 radiation).

In some embodiments, the 2C-B Xinafoate salt is a crystalline polymorph characterized by two or more, or three XRPD signals selected from the group consisting of 24.8 °2θ, 23.3 °2θ, 18.6 °2θ, and 7.9 °2θ (±0.2 °2θ; ±0.1 °2θ; or ±0.0 °2θ; Cu Kα1 radiation).

In some embodiments, the 2C-B Xinafoate salt is a crystalline polymorph characterized by two or more, or three XRPD signals selected from the group consisting of 24.8 °2θ, 23.3 °2θ, 18.6 °2θ, 7.9 °2θ, and 24.9 °2θ (±0.2 °2θ; ±0.1 °2θ; or ±0.0 °2θ; Cu Kα1 radiation).

In some embodiments, the 2C-B Xinafoate salt is a crystalline polymorph characterized by two or more, or three XRPD signals selected from the group consisting of 24.8 °2θ, 23.3 °2θ, 18.6 °2θ, 7.9 °2θ, 24.9 °2θ, 20.1 °2θ, 22.5 °2θ, 26.2 °2θ, 13.9 °2θ, and 14.4 °2θ (±0.2 °2θ; ±0.1 °2θ; or ±0.0 °2θ; Cu Kα1 radiation).

In some embodiments, the 2C-B Xinafoate salt is crystalline polymorphic characterized by two, three, four, five, six, seven, eight, nine, or ten XRPD signals as shown in Table 69.

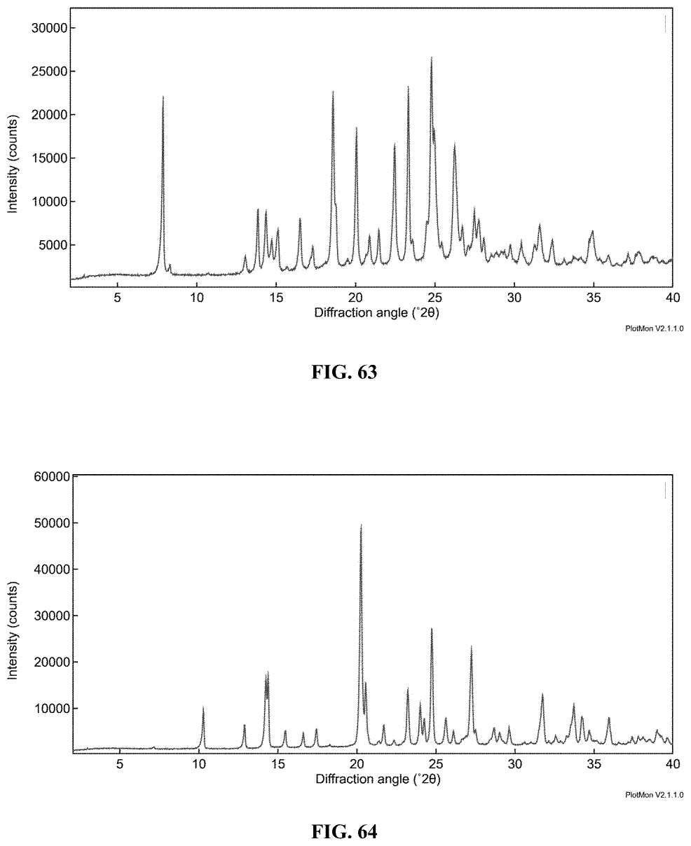

In some embodiments, the 2C-B Xinafoate salt is a crystalline polymorph characterized by two or more, or three XRPD signals as shown in FIG. 63.

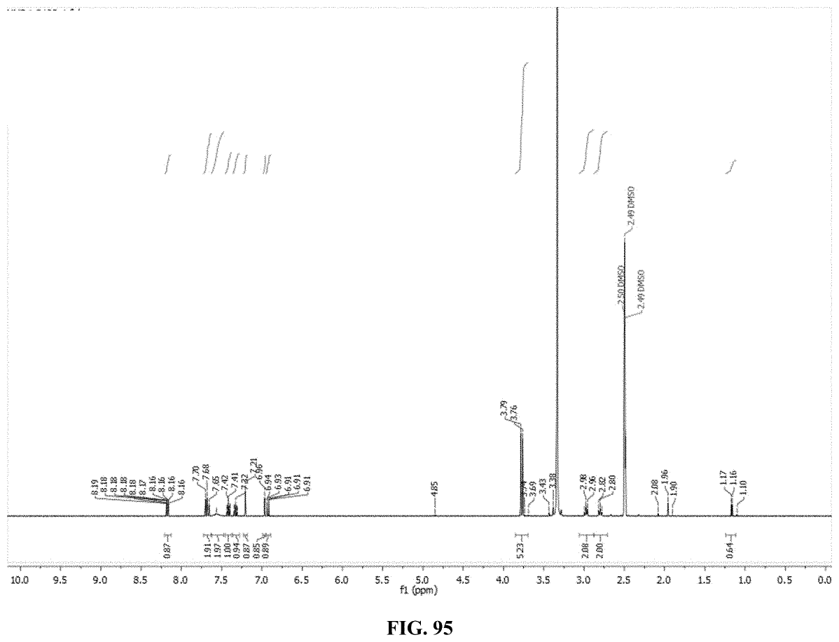

In some embodiments, 2C-B Xinafoate has an ¹H NMR spectra as provided in FIG. 95.

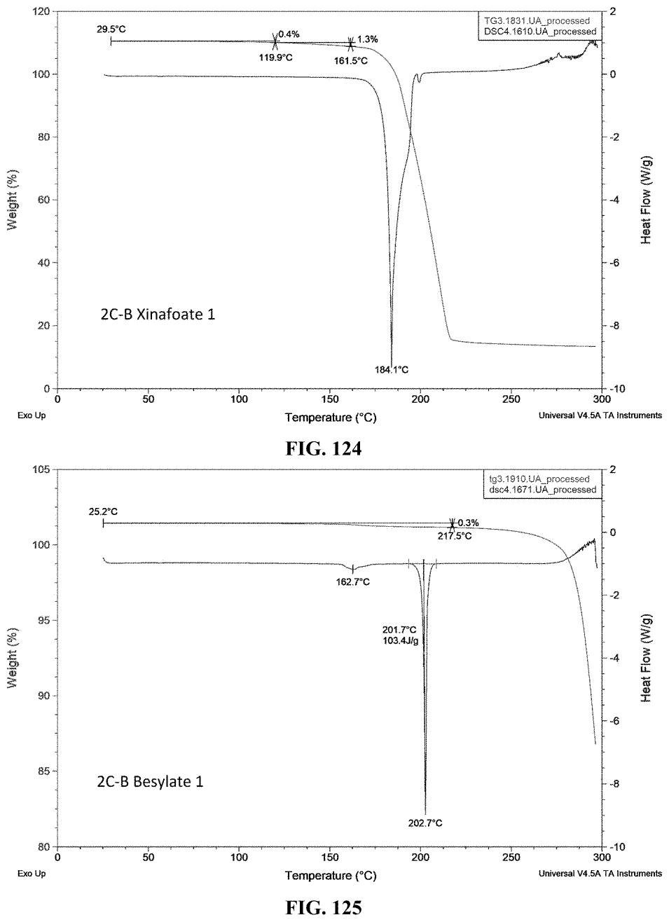

In some embodiments, 2C-B Xinafoate has TGA and DSC profiles as provided in FIG. 124.

### 2C-B Lactate Salt

In some embodiments, the 2C-B Lactate salt is crystalline polymorphic (Form 1) characterized by two or more, or three XRPD signals selected from the group consisting of 7.1 °2θ, 23.2 °2θ, and 25.3 °2θ (±0.2 °2θ; ±0.1 °2θ; or ±0.0 °2θ; Cu Kα1 radiation).

In some embodiments, the 2C-B Lactate salt is crystalline polymorphic (Form 1) characterized by two or more, or three XRPD signals selected from the group consisting of 7.1 °2θ, 23.2 °2θ, 25.3 °2θ, and 8.1 °2θ (±0.2 °2θ; ±0.1 °2θ; or ±0.0 °2θ; Cu Kα1 radiation).

In some embodiments, the 2C-B Lactate salt is crystalline polymorphic (Form 1) characterized by two or more, or three XRPD signals selected from the group consisting of 7.1 °2θ, 23.2 °2θ, 25.3 °2θ, 8.1 °2θ, and 10.7 °2θ (±0.2 °2θ; ±0.1 °2θ; or ±0.0 °2θ; Cu Kα1 radiation).

In some embodiments, the 2C-B Lactate salt is crystalline polymorphic (Form 1) characterized by two or more, or three XRPD signals selected from the group consisting of 7.1 °2θ, 23.2 °2θ, 25.3 °2θ, 8.1 °2θ, 10.7 °2θ, 24 °2θ, 26.3 °2θ, 29 °2θ, 15.3 °2θ, and 26.6 °2θ (±0.2 °2θ; ±0.1 °2θ; or ±0.0 °2θ; Cu Kα1 radiation).

In some embodiments, the 2C-B Lactate salt is crystalline polymorphic (Form 1) characterized by two, three, four, five, six, seven, eight, nine, or ten XRPD signals as shown in Table 29.

In some embodiments, the 2C-B Lactate salt is crystalline polymorphic (Form 1) characterized by two or more, or three XRPD signals as shown in FIG. 18.

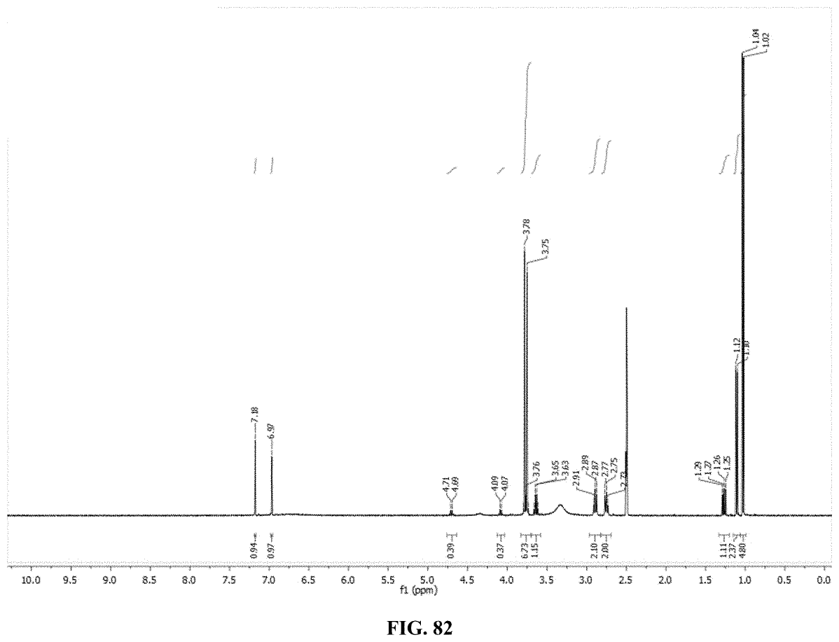

In some embodiments, 2C-B Lactate (Form 1) has an ¹H NMR spectra as provided in FIG. 82.

In some embodiments, the 2C-B Lactate (Form 1) is a crystalline polymorph characterized by two or more, or three XRPD signals selected from the group consisting of 7.1 °2θ, 8.1 °2θ, and 10.7 °2θ (±0.2 °2θ; ±0.1 °2θ; or ±0.0 °2θ; Cu Kα1 radiation).

In some embodiments, the 2C-B Lactate (Form 1) is a crystalline polymorph characterized by two or more, or three XRPD signals selected from the group consisting of 7.1 °2θ, 8.1 °2θ, 10.7 °2θ, 15.3 °2θ, and 23.2 °2θ (±0.2 °2θ; ±0.1 °2θ; or ±0.0 °2θ; Cu Kα1 radiation).

In some embodiments, the 2C-B Lactate salt is crystalline polymorphic (Form 2) characterized by two or more, or three XRPD signals selected from the group consisting of 19.4 °2θ, 25.2 °2θ, and 10.5 °2θ (±0.2 °2θ; ±0.1 °2θ; or ±0.0 °2θ; Cu Kα1 radiation).

In some embodiments, the 2C-B Lactate salt is crystalline polymorphic (Form 2) characterized by two or more, or three XRPD signals selected from the group consisting of 19.4 °2θ, 25.2 °2θ, 10.5 °2θ, and 5.3 °2θ (±0.2 °2θ; ±0.1 °2θ; or ±0.0 °2θ; Cu Kα1 radiation).

In some embodiments, the 2C-B Lactate salt is crystalline polymorphic (Form 2) characterized by two or more, or three XRPD signals selected from the group consisting of 19.4 °2θ, 25.2 °2θ, 10.5 °2θ, 5.3 °2θ, and 16.3 °2θ (±0.2 °2θ; ±0.1 °2θ; or ±0.0 °2θ; Cu Kα1 radiation).

In some embodiments, the 2C-B Lactate salt is crystalline polymorphic (Form 2) characterized by two or more, or three XRPD signals selected from the group consisting of 19.4 °2θ, 25.2 °2θ, 10.5 °2θ, 5.3 °2θ, 16.3 °2θ, 23.1 °2θ, 26.4 °2θ, 15.5 °2θ, 7.4 °2θ, and 31.1 °2θ (±0.2 °2θ; ±0.1 °2θ; or ±0.0 °2θ; Cu Kα1 radiation).

In some embodiments, the 2C-B Lactate salt is crystalline polymorphic (Form 2) characterized by two, three, four, five, six, seven, eight, nine, or ten XRPD signals as shown in Table 41.

In some embodiments, the 2C-B Lactate salt is crystalline polymorphic (Form 2) characterized by two or more, or three XRPD signals as shown in FIG. 32.

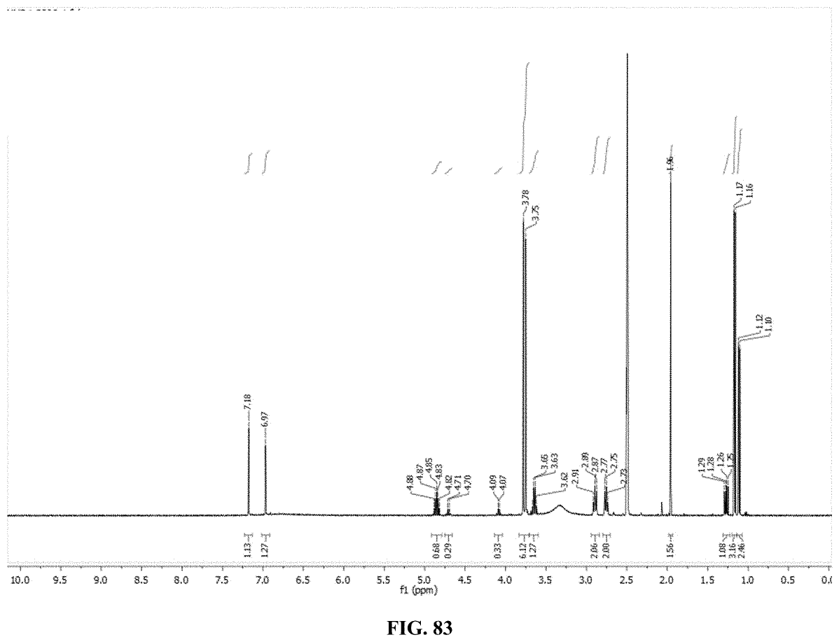

In some embodiments, 2C-B Lactate (Form 2) has an ¹H NMR spectra as provided in FIG. 83.

In some embodiments, the 2C-B Lactate (Form 2) is a crystalline polymorph characterized by two or more, or three XRPD signals selected from the group consisting of 5.3 °2θ, 7.4 °2θ, and 10.5 °2θ (±0.2 °2θ; ±0.1 °2θ; or ±0.0 °2θ; Cu Kα1 radiation).

In some embodiments, the 2C-B Lactate (Form 2) is a crystalline polymorph characterized by two or more, or three XRPD signals selected from the group consisting of 5.3 °2θ, 7.4 °2θ, 10.5 °2θ, 15.5 °2θ, and 19.4 °2θ (±0.2 °2θ; ±0.1 °2θ; or ±0.0 °2θ; Cu Kα1 radiation).

### 2C-B Malate Salt

In some embodiments, the 2C-B malate salt is crystalline polymorphic (malate+peaks) characterized by two or more, or three XRPD signals selected from the group consisting of 23.9 °2θ, 24.4 °2θ, and 19.8 °2θ (±0.2 °2θ; ±0.1 °2θ; or ±0.0 °2θ; Cu Kα1 radiation). The XRPD signals include the signals corresponding to 2C-B malate and other signals.

In some embodiments, the 2C-B malate salt is crystalline polymorphic (malate+peaks) characterized by two or more, or three XRPD signals selected from the group consisting of 23.9 °2θ, 24.4 °2θ, 19.8 °2θ, and 23.3 °2θ (±0.2 °2θ; ±0.1 °2θ; or ±0.0 °2θ; Cu Kα1 radiation). The XRPD signals include the signals corresponding to 2C-B malate and other signals.

In some embodiments, the 2C-B malate salt is crystalline polymorphic (malate+peaks) characterized by two or more, or three XRPD signals selected from the group consisting of 23.9 °2θ, 24.4 °2θ, 19.8 °2θ, 23.3 °2θ, and 16.5 °2θ (±0.2 °2θ; ±0.1 °2θ; or ±0.0 °2θ; Cu Kα1 radiation). The XRPD signals include the signals corresponding to 2C-B malate and other signals.

In some embodiments, the 2C-B malate salt is crystalline polymorphic (malate+peaks) characterized by two or more, or three XRPD signals selected from the group consisting of 23.9 °2θ, 24.4 °2θ, 19.8 °2θ, 23.3 °2θ, 16.5 °2θ, 17.8 °2θ, 25.5 °2θ, 12 °2θ, 17.5 °2θ, and 34.8 °2θ (±0.2 °2θ; ±0.1 °2θ; or ±0.0 °2θ; Cu Kα1 radiation). The XRPD signals include the signals corresponding to 2C-B malate and other signals.

In some embodiments, the 2C-B malate salt is crystalline polymorphic (malate+peaks) characterized by two or more, or three XRPD signals selected from the group consisting of 12.0 °2θ, 14.6 °2θ, and 16.5 °2θ (±0.2 °2θ; ±0.1 °2θ; or ±0.0 °2θ; Cu Kα1 radiation). The XRPD signals include the signals corresponding to 2C-B malate and other signals.

In some embodiments, the 2C-B malate salt is crystalline polymorphic (malate+peaks) characterized by two or more, or three XRPD signals selected from the group consisting of 12.0 °2θ, 14.6 °2θ, 16.5 °2θ, 17.8 °2θ, and 19.8 °2θ (±0.2 °2θ; ±0.1 °2θ; or ±0.0 °2θ; Cu Kα1 radiation). The XRPD signals include the signals corresponding to 2C-B malate and other signals.

In some embodiments, the 2C-B malate salt is crystalline polymorphic (malate+peaks) characterized by two, three, four, five, six, seven, eight, nine, or ten XRPD signals as shown in Table 30. The XRPD signals include the signals corresponding to 2C-B malate and other signals.

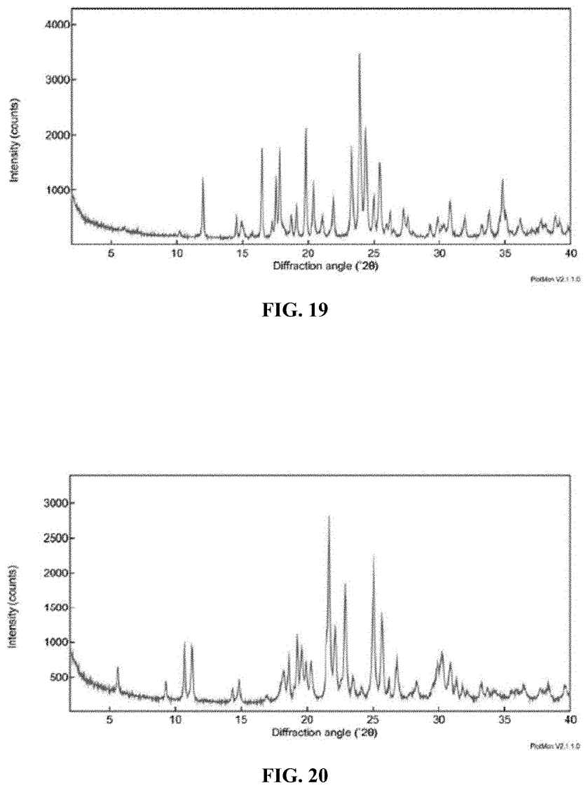

In some embodiments, the 2C-B malate salt is crystalline polymorphic (malate+peaks) characterized by two or more, or three XRPD signals as shown in FIG. 19. The XRPD signals include the signals corresponding to 2C-B malate and other signals.

In some embodiments, the 2C-B Malate is a crystalline polymorph characterized by two or more, or three XRPD signals selected from the group consisting of 23.9 °2θ, 24.4 °2θ, and 19.8 °2θ (±0.2 °2θ; ±0.1 °2θ; or ±0.0 °2θ; Cu Kα1 radiation).

In some embodiments, the 2C-B Malate is a crystalline polymorph characterized by two or more, or three XRPD signals selected from the group consisting of 23.9 °2θ, 24.4 °2θ, 19.8 °2θ, and 17.9 °2θ (±0.2 °2θ; ±0.1 °2θ; or ±0.0 °2θ; Cu Kα1 radiation).

In some embodiments, the 2C-B Malate is a crystalline polymorph characterized by two or more, or three XRPD signals selected from the group consisting of 23.9 °2θ, 24.4 °2θ, 19.8 °2θ, 17.9 °2θ, and 16.5 °2θ (±0.2 °2θ; ±0.1 °2θ; or ±0.0 °2θ; Cu Kα1 radiation).

In some embodiments, the 2C-B Malate is a crystalline polymorph characterized by two or more, or three XRPD signals selected from the group consisting of 23.9 °2θ, 24.4 °2θ, 19.8 °2θ, 17.9 °2θ, 16.5 °2θ, 23.3 °2θ, 25.5 °2θ, 34.8 °2θ, 12 °2θ, and 17.6 °2θ (±0.2 °2θ; ±0.1 °2θ; or ±0.0 °2θ; Cu Kα1 radiation).

In some embodiments, the 2C-B Malate is a crystalline polymorph characterized by two or more, or three XRPD signals selected from the group consisting of 12.0 °2θ, 14.6 °2θ, and 16.5 °2θ (±0.2 °2θ; ±0.1 °2θ; or ±0.0 °2θ; Cu Kα1 radiation).

In some embodiments, the 2C-B Malate is a crystalline polymorph characterized by two or more, or three XRPD signals selected from the group consisting of 12.0 °2θ, 14.6 °2θ, 16.5 °2θ, 17.9 °2θ, and 19.8 °2θ (±0.2 °2θ; ±0.1 °2θ; or ±0.0 °2θ; Cu Kα1 radiation).

In some embodiments, the 2C-B Malate is a crystalline polymorph characterized by two, three, four, five, six, seven, eight, nine, or ten XRPD signals as shown in Table 66.

In some embodiments, the 2C-B Malate is a crystalline polymorph characterized by two or more, or three XRPD signals as shown in FIG. 60.

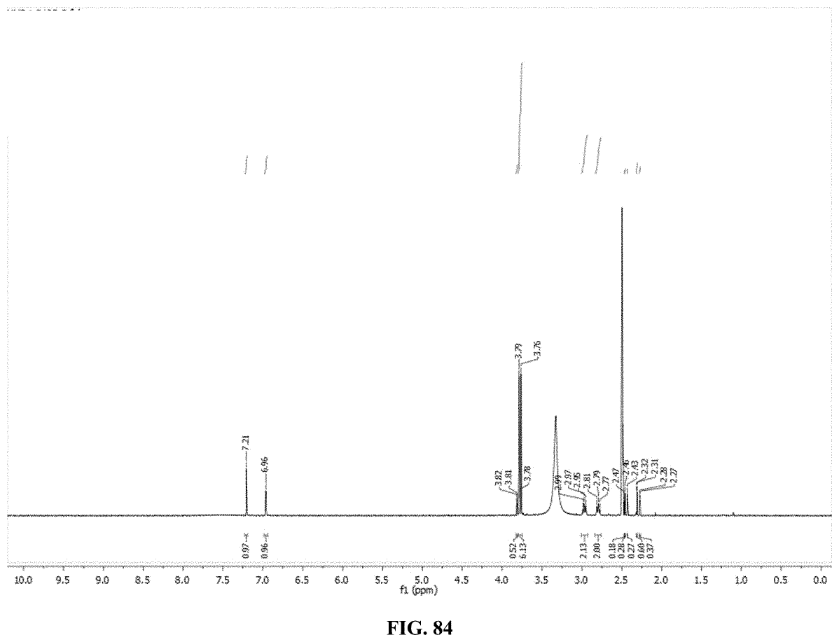

In some embodiments, 2C-B Malate has an ¹H NMR spectra as provided in FIG. 84.

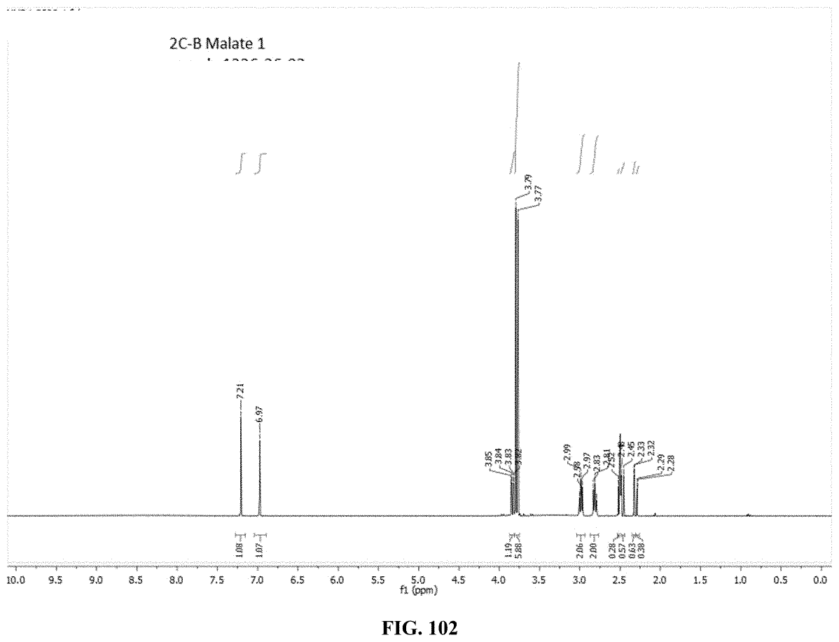

In some embodiments, 2C-B Malate has an ¹H NMR spectra as provided in FIG. 102.

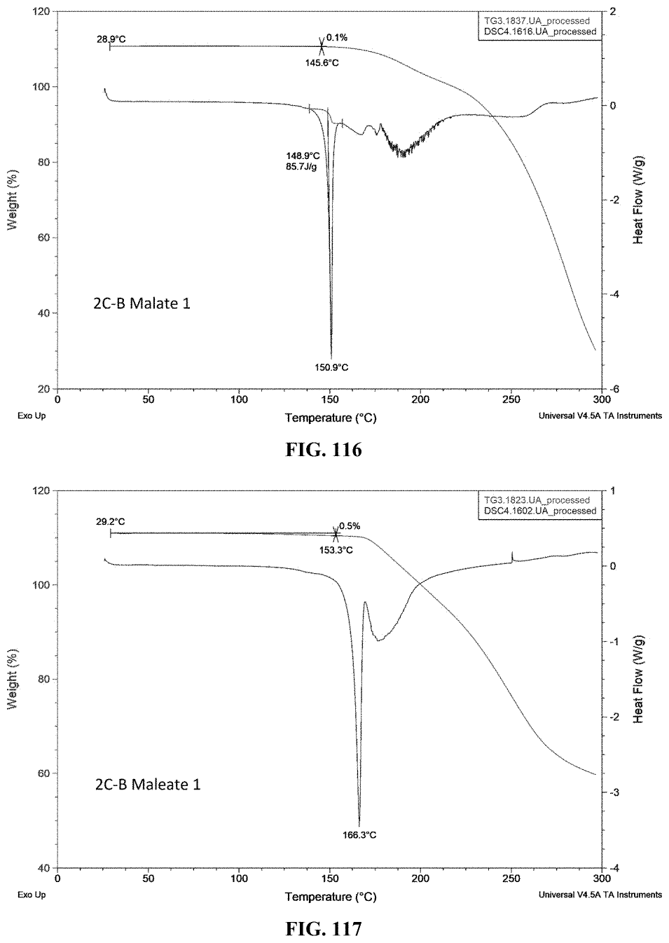

In some embodiments, 2C-B Malate has TGA and DSC profiles as provided in FIG. 116.

In some embodiments, 2C-B Malate has TGA and DSC profiles as provided in FIG. 129.

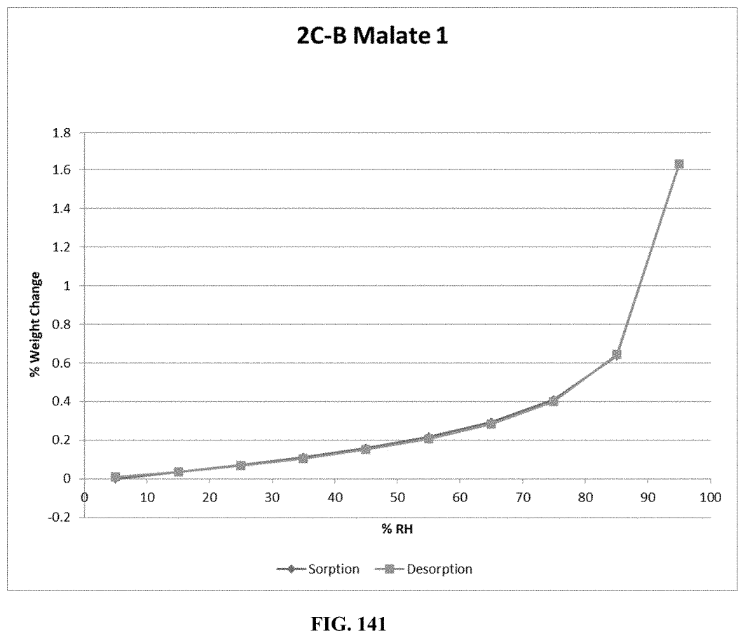

In some embodiments, 2C-B Malate has a DVS profile as provided in FIG. 141.

In some embodiments, the 2C-B malate salt possesses at least one of the following characteristics:

(a) exhibit high crystallinity,

(b) exhibit acceptable scale-up

(c) exhibit minimal water uptake over RH range,

(d) exhibit uncomplicated DSC data (one endothermic event observed), and

(e) acceptable solubility when compared to the other salts.

In some embodiments, the 2C-B malate salt possesses at least one of characteristic as provided in Table 86.

### 2C-B Maleate Salt

In some embodiments, the 2C-B Maleate salt is crystalline polymorphic (Form 1) characterized by two or more, or three XRPD signals selected from the group consisting of 21.7 °2θ, 25.1 °2θ, and 22.9 °2θ (±0.2 °2θ; ±0.1 °2θ; or ±0.0 °2θ; Cu Kα1 radiation).

In some embodiments, the 2C-B Maleate salt is crystalline polymorphic (Form 1) characterized by two or more, or three XRPD signals selected from the group consisting of 21.7 °2θ, 25.1 °2θ, 22.9 °2θ, and 25.7 °2θ (±0.2 °2θ; ±0.1 °2θ; or ±0.0 °2θ; Cu Kα1 radiation). In some embodiments, the 2C-B Maleate salt is crystalline polymorphic (Form 1) characterized by two or more, or three XRPD signals selected from the group consisting of 21.7 °2θ, 25.1 °2θ, 22.9 °2θ, 25.7 °2θ, and 22.1 °2θ (±0.2 °2θ; ±0.1 °2θ; or ±0.0 °2θ; Cu Kα1 radiation).

In some embodiments, the 2C-B Maleate salt is crystalline polymorphic (Form 1) characterized by two or more, or three XRPD signals selected from the group consisting of 21.7 °2θ, 25.1 °2θ, 22.9 °2θ, 25.7 °2θ, 22.1 °2θ, 19.3 °2θ, 10.7 °2θ, 11.2 °2θ, 19.6 °2θ, and 30.2 °2θ (±0.2 °2θ; ±0.1 °2θ; or ±0.0 °2θ; Cu Kα1 radiation).

In some embodiments, the 2C-B Maleate salt is crystalline polymorphic (Form 1) characterized by two, three, four, five, six, seven, eight, nine, or ten XRPD signals as shown in Table 31.

In some embodiments, the 2C-B Maleate salt is crystalline polymorphic (Form 1) characterized by two or more, or three XRPD signals as shown in FIG. 20.

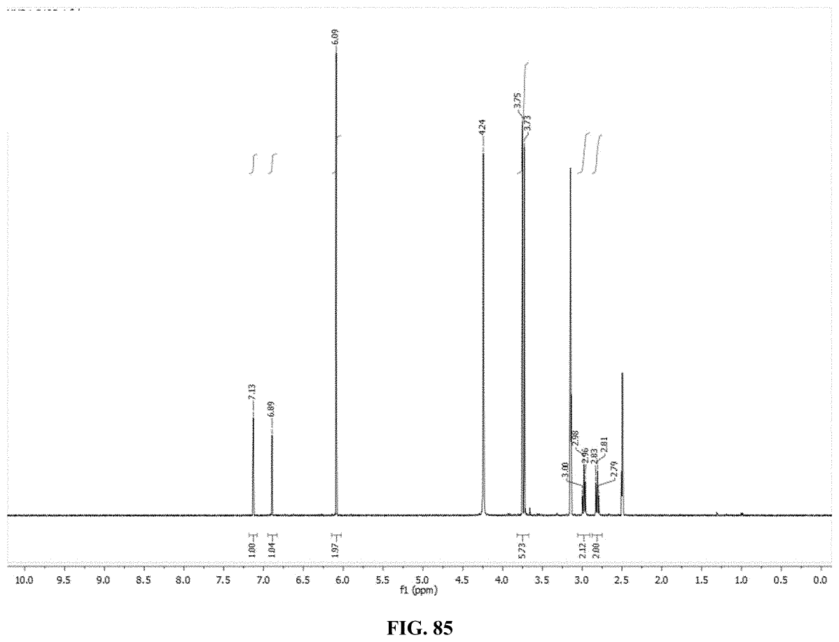

In some embodiments, 2C-B Maleate (Form 1) has an ¹H NMR spectra as provided in FIG. 85.

In some embodiments, 2C-B Maleate (Form 1) has TGA and DSC profiles as provided in FIG. 117.

In some embodiments, the 2C-B Maleate (Form 1) is a crystalline polymorph characterized by two or more, or three XRPD signals selected from the group consisting of 5.7 °2θ, 10.7 °2θ, and 11.2 °2θ (±0.2 °2θ; ±0.1 °2θ; or ±0.0 °2θ; Cu Kα1 radiation).

In some embodiments, the 2C-B Maleate (Form 1) is a crystalline polymorph characterized by two or more, or three XRPD signals selected from the group consisting of 5.7 °2θ, 10.7 °2θ, 11.2 °2θ, 9.3 °2θ, and 14.8 °2θ (±0.2 °2θ; ±0.1 °2θ; or ±0.0 °2θ; Cu Kα1 radiation).

In some embodiments, the 2C-B Maleate salt is crystalline polymorphic (Form 2) characterized by two or more, or three XRPD signals selected from the group consisting of 25.9 °2θ, 26.2 °2θ, and 25.6 °2θ (±0.2 °2θ; ±0.1 °2θ; or ±0.0 °2θ; Cu Kα1 radiation).

In some embodiments, the 2C-B Maleate salt is crystalline polymorphic (Form 2) characterized by two or more, or three XRPD signals selected from the group consisting of 25.9 °2θ, 26.2 °2θ, 25.6 °2θ, and 23.8 °2θ (±0.2 °2θ; ±0.1 °2θ; or ±0.0 °2θ; Cu Kα1 radiation).

In some embodiments, the 2C-B Maleate salt is crystalline polymorphic (Form 2) characterized by two or more, or three XRPD signals selected from the group consisting of 25.9 °2θ, 26.2 °2θ, 25.6 °2θ, 23.8 °2θ, and 18.2 °2θ (±0.2 °2θ; ±0.1 °2θ; or ±0.0 °2θ; Cu Kα1 radiation).

In some embodiments, the 2C-B Maleate salt is crystalline polymorphic (Form 2) characterized by two or more, or three XRPD signals selected from the group consisting of 25.9 °2θ, 26.2 °2θ, 25.6 °2θ, 23.8 °2θ, 18.2 °2θ, 15.2 °2θ, 27.8 °2θ, 10.1 °2θ, 19.9 °2θ, and 29.2 °2θ (±0.2 °2θ; ±0.1 °2θ; or ±0.0 °2θ; Cu Kα1 radiation).

In some embodiments, the 2C-B Maleate salt is crystalline polymorphic (Form 2) characterized by two, three, four, five, six, seven, eight, nine, or ten XRPD signals as shown in Table 42.

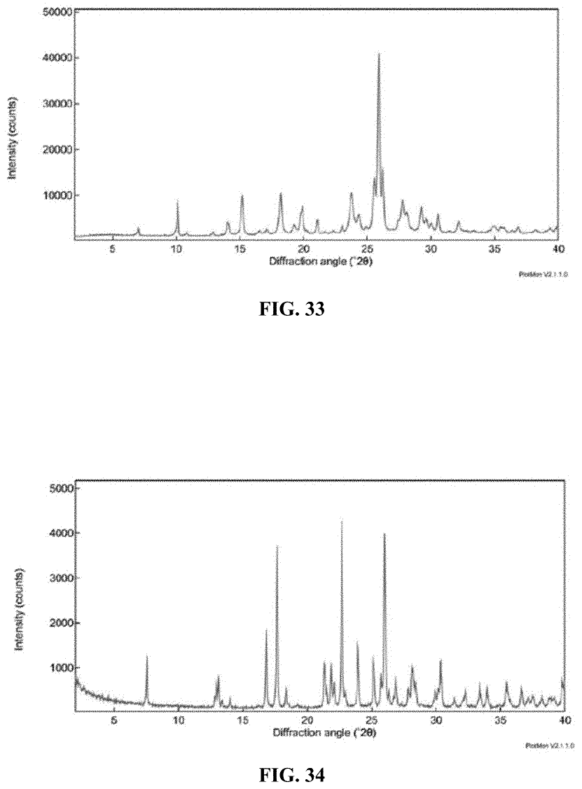

In some embodiments, the 2C-B Maleate salt is crystalline polymorphic (Form 2) characterized by two or more, or three XRPD signals as shown in FIG. 33.

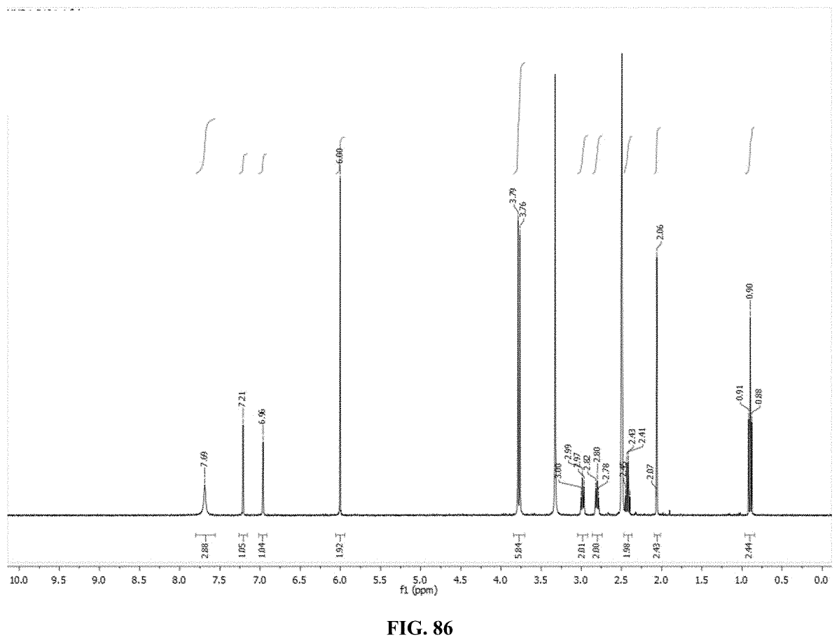

In some embodiments, 2C-B Maleate (Form 2) has an ¹H NMR spectra as provided in FIG. 86.

In some embodiments, the 2C-B Maleate (Form 2) is a crystalline polymorph characterized by two or more, or three XRPD signals selected from the group consisting of 10.1 °2θ, 14.0 °2θ, and 15.2 °2θ (±0.2 °2θ; ±0.1 °2θ; or ±0.0 °2θ; Cu Kα1 radiation).

In some embodiments, the 2C-B Maleate (Form 2) is a crystalline polymorph characterized by two or more, or three XRPD signals selected from the group consisting of 10.1 °2θ, 14.0 °2θ, 15.2 °2θ, 18.2 °2θ, and 23.8 °2θ (±0.2 °2θ; ±0.1 °2θ; or ±0.0 °2θ; Cu Kα1 radiation).

### 2C-B Malonate Salt

In some embodiments, the 2C-B malonate salt is a crystalline polymorph characterized by two or more, or three XRPD signals selected from the group consisting of 23.2 °2θ, 26.5 °2θ, and 16 °2θ (±0.2 °2θ; ±0.1 °2θ; or ±0.0 °2θ; Cu Kα1 radiation).

In some embodiments, the 2C-B malonate salt is a crystalline polymorph characterized by two or more, or three XRPD signals selected from the group consisting of 23.2 °2θ, 26.5 °2θ, 16 °2θ, and 12.3 °2θ (±0.2 °2θ; ±0.1 °2θ; or ±0.0 °2θ; Cu Kα1 radiation).

In some embodiments, the 2C-B malonate salt is a crystalline polymorph characterized by two or more, or three XRPD signals selected from the group consisting of 23.2 °2θ, 26.5 °2θ, 16 °2θ, 12.3 °2θ, and 10.2 °2θ (±0.2 °2θ; ±0.1 °2θ; or ±0.0 °2θ; Cu Kα1 radiation).

In some embodiments, the 2C-B malonate salt is a crystalline polymorph characterized by two or more, or three XRPD signals selected from the group consisting of 23.2 °2θ, 26.5 °2θ, 16 °2θ, 12.3 °2θ, 10.2 °2θ, 21.7 °2θ, 25.4 °2θ, 25.7 °2θ, 25.1 °2θ, and 5.1 °2θ (±0.2 °2θ; ±0.1 °2θ; or ±0.0 °2θ; Cu Kα1 radiation).

In some embodiments, the 2C-B malonate salt is a crystalline polymorph characterized by two, three, four, five, six, seven, eight, nine, or ten XRPD signals as shown in Table 32.

In some embodiments, the 2C-B malonate salt is a crystalline polymorph characterized by two or more, or three XRPD signals as shown in FIG. 21.

In some embodiments, the 2C-B malonate salt is a crystalline polymorph characterized by two or more, or three XRPD signals selected from the group consisting of 5.1 °2θ, 10.2 °2θ, and 12.3 °2θ (±0.2 °2θ; ±0.1 °2θ; or ±0.0 °2θ; Cu Kα1 radiation).

In some embodiments, the 2C-B malonate salt is a crystalline polymorph characterized by two or more, or three XRPD signals selected from the group consisting of 5.1 °2θ, 10.2 °2θ, 12.3 °2θ, 9.9 °2θ, and 16 °2θ (±0.2 °2θ; ±0.1 °2θ; or ±0.0 °2θ; Cu Kα1 radiation).

### 2C-B Mesylate Salt

In some embodiments, the 2C-B Mesylate salt is a crystalline polymorph characterized by two or more, or three XRPD signals selected from the group consisting of 25.9 °2θ, 22.5 °2θ, 19.1 °2θ, and 25.6 °2θ (±0.2 °2θ; ±0.1 °2θ; or ±0.0 °2θ; Cu Kα1 radiation).

In some embodiments, the 2C-B Mesylate salt is a crystalline polymorph characterized by two or more, or three XRPD signals selected from the group consisting of 25.9 °2θ, 22.5 °2θ, 19.1 °2θ, 25.6 °2θ, and 11.4 °2θ (±0.2 °2θ; ±0.1 °2θ; or ±0.0 °2θ; Cu Kα1 radiation).

In some embodiments, the 2C-B Mesylate salt is crystalline polymorphic characterized by two or more, or three XRPD signals selected from the group consisting of 25.9 °2θ, 22.5 °2θ, 19.1 °2θ, 25.6 °2θ, 11.4 °2θ, 17.6 °2θ, 25.7 °2θ, 21.3 °2θ, 22.7 °2θ, and 20.1 °2θ (±0.2 °2θ; ±0.1 °2θ; or ±0.0 °2θ; Cu Kα1 radiation).

In some embodiments, the 2C-B Mesylate salt is crystalline polymorphic characterized by two or more, or three XRPD signals selected from the group consisting of 5.7 °2θ, 11.4 °2θ, and 15.4 °2θ (±0.2 °2θ; ±0.1 °2θ; or ±0.0 °2θ; Cu Kα1 radiation).

In some embodiments, the 2C-B Mesylate salt is a crystalline polymorph characterized by two or more, or three XRPD signals selected from the group consisting of 5.7 °2θ, 11.4 °2θ, 15.4 °2θ, 17.6 °2θ, and 19.1 (±0.2 °2θ; ±0.1 °2θ; or ±0.0 °2θ; Cu Kα1 radiation).

In some embodiments, the 2C-B Mesylate salt is a crystalline polymorph characterized by two, three, four, five, six, seven, eight, nine, or ten XRPD signals as shown in Table 33.

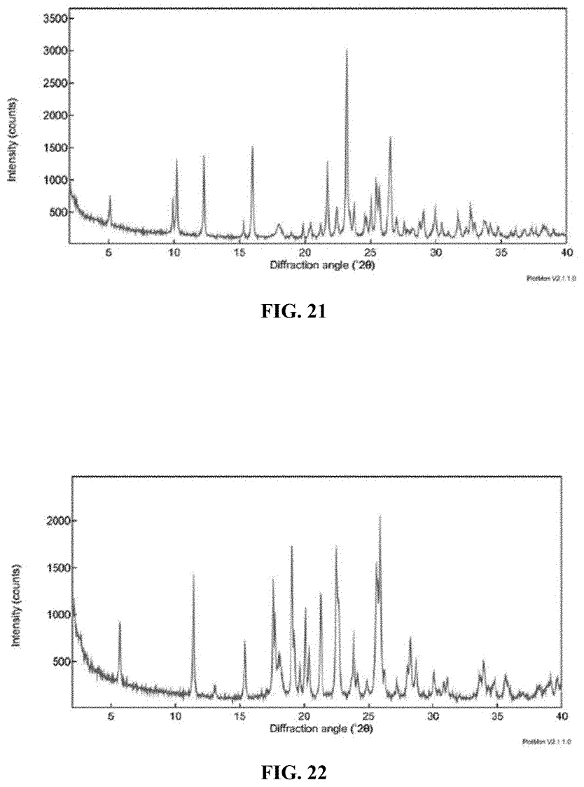

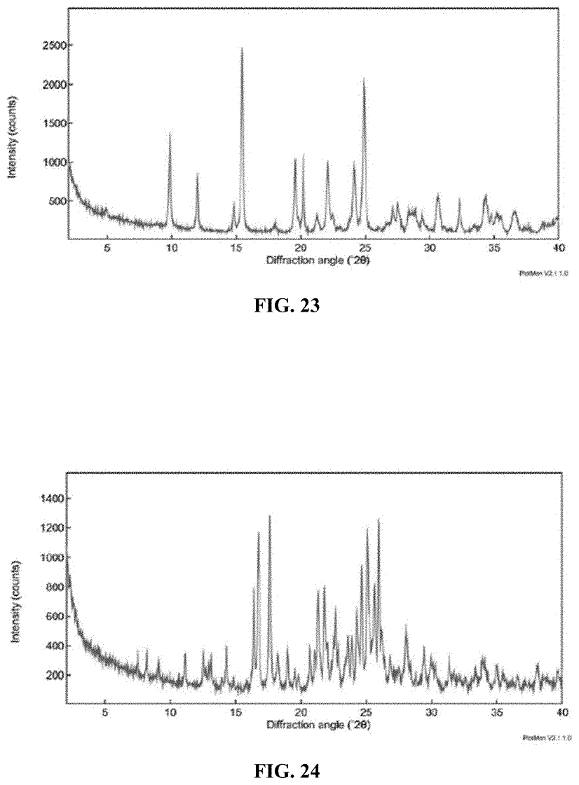

In some embodiments, the 2C-B Mesylate salt is a crystalline polymorph characterized by two or more, or three XRPD signals as shown in FIG. 22.

In some embodiments, the 2C-B Mesylate is a crystalline polymorph characterized by two or more, or three XRPD signals selected from the group consisting of 5.8 °2θ, 11.4 °2θ, and 15.5 °2θ (±0.2 °2θ; ±0.1 °2θ; or ±0.0 °2θ; Cu Kα1 radiation).

In some embodiments, the 2C-B Mesylate is a crystalline polymorph characterized by two or more, or three XRPD signals selected from the group consisting of 5.8 °2θ, 11.4 °2θ, 15.5 °2θ, 17.6 °2θ, and 19.1 °2θ (±0.2 °2θ; ±0.1 °2θ; or ±0.0 °2θ; Cu Kα1 radiation).

In some embodiments, the 2C-B Mesylate salt is a crystalline polymorph characterized by two or more, or three XRPD signals selected from the group consisting of 25.9 °2θ, 19.1 °2θ, and 22.5 °2θ (±0.2 °2θ; ±0.1 °2θ; or ±0.0 °2θ; Cu Kα1 radiation).

In some embodiments, the 2C-B Mesylate salt is a crystalline polymorph characterized by two or more, or three XRPD signals selected from the group consisting of 25.9 °2θ, 19.1 °2θ, 22.5 °2θ, and 11.4 °2θ (±0.2 °2θ; ±0.1 °2θ; or ±0.0 °2θ; Cu Kα1 radiation).

In some embodiments, the 2C-B Mesylate salt is a crystalline polymorph characterized by two or more, or three XRPD signals selected from the group consisting of 25.9 °2θ, 19.1 °2θ, 22.5 °2θ, 11.4 °2θ, and 25.6 °2θ (±0.2 °2θ; ±0.1 °2θ; or ±0.0 °2θ; Cu Kα1 radiation).

In some embodiments, the 2C-B Mesylate salt is a crystalline polymorph characterized by two or more, or three XRPD signals selected from the group consisting of 25.9 °2θ, 19.1 °2θ, 22.5 °2θ, 11.4 °2θ, 25.6 °2θ, 17.6 °2θ, 21.3 °2θ, 22.7 °2θ, 22.6 °2θ, and 20.1 °2θ (±0.2 °2θ; ±0.1 °2θ; or ±0.0 °2θ; Cu Kα1 radiation).

In some embodiments, the 2C-B Mesylate salt is a crystalline polymorph characterized by two, three, four, five, six, seven, eight, nine, or ten XRPD signals as shown in Table 67.

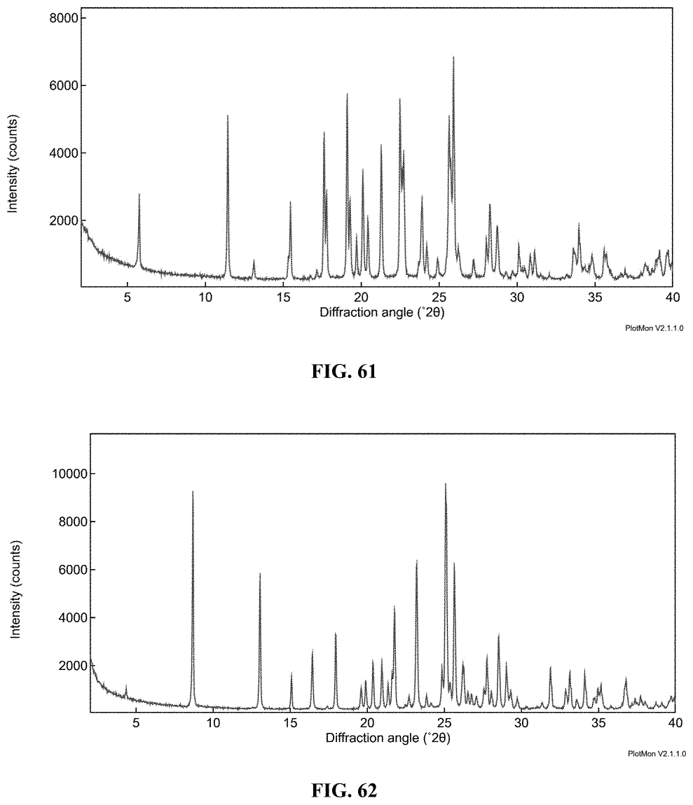

In some embodiments, the 2C-B Mesylate salt is a crystalline polymorph characterized by two or more, or three XRPD signals as shown in FIG. 61.

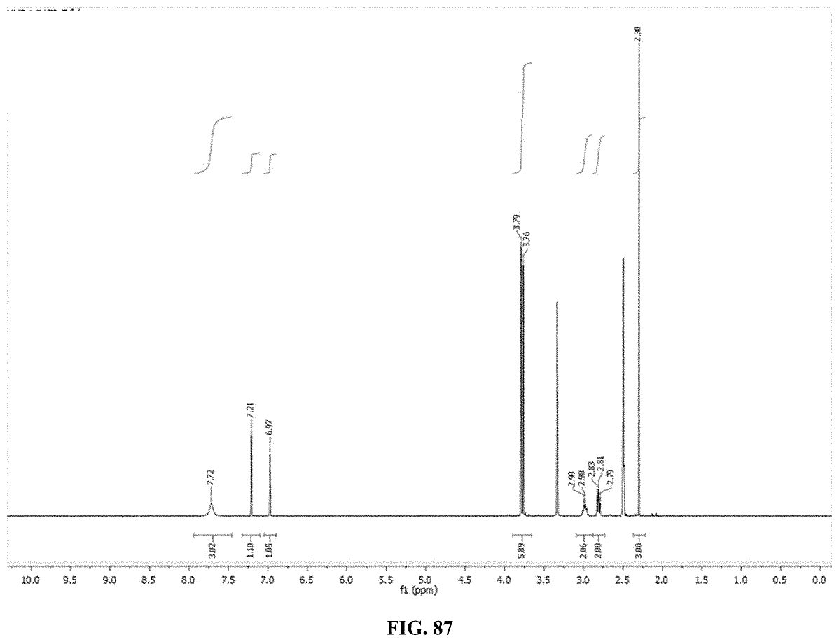

In some embodiments, 2C-B Mesylate has an ¹H NMR spectra as provided in FIG. 87.

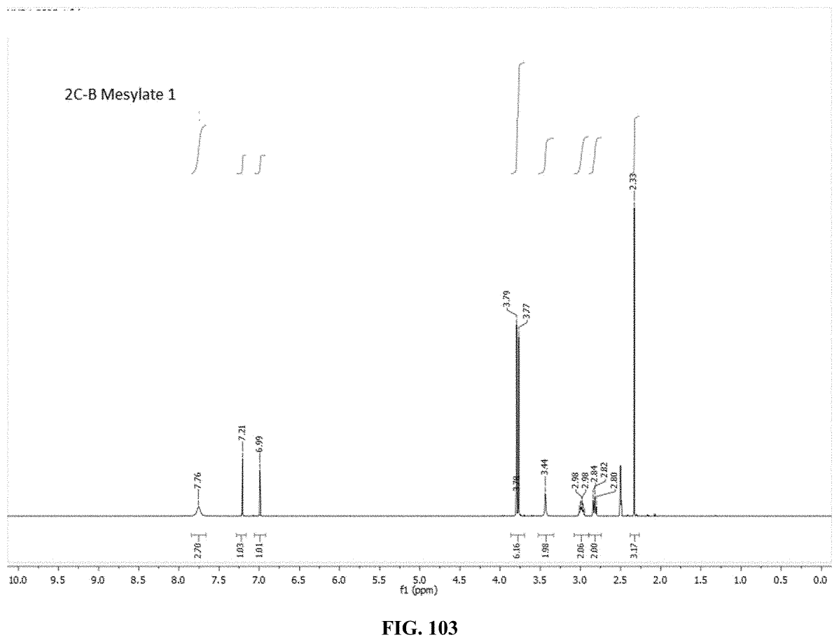

In some embodiments, 2C-B Mesylate has an ¹H NMR spectra as provided in FIG. 103.

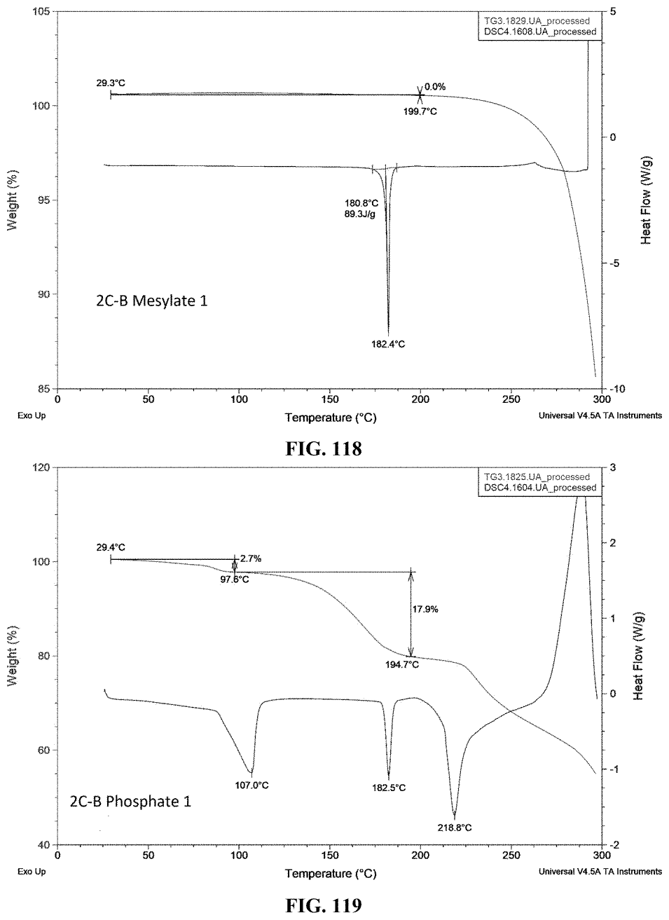

In some embodiments, 2C-B Mesylate has TGA and DSC profiles as provided in FIG. 118.

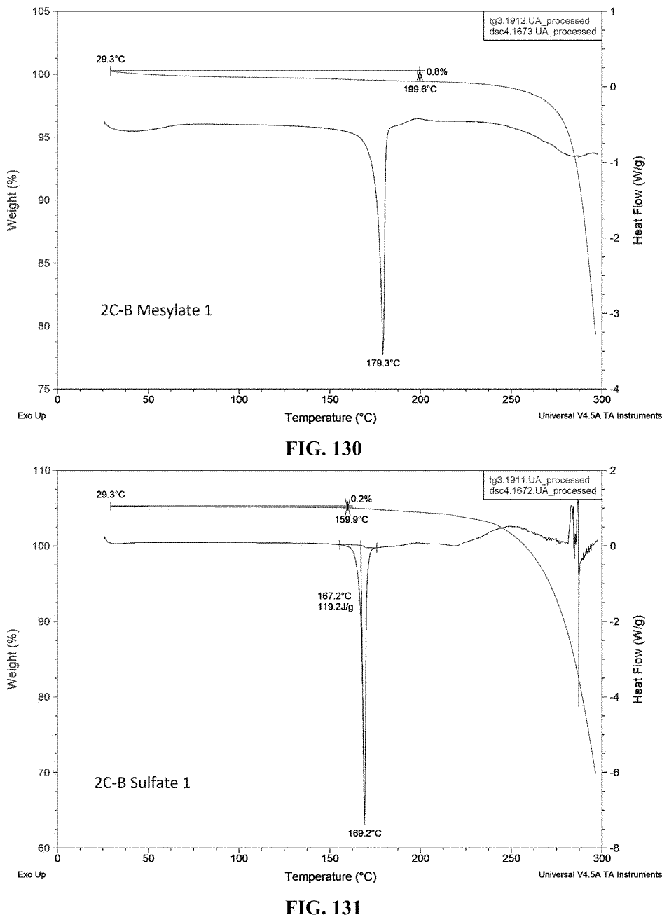

In some embodiments, 2C-B Mesylate has TGA and DSC profiles as provided in FIG. 130.

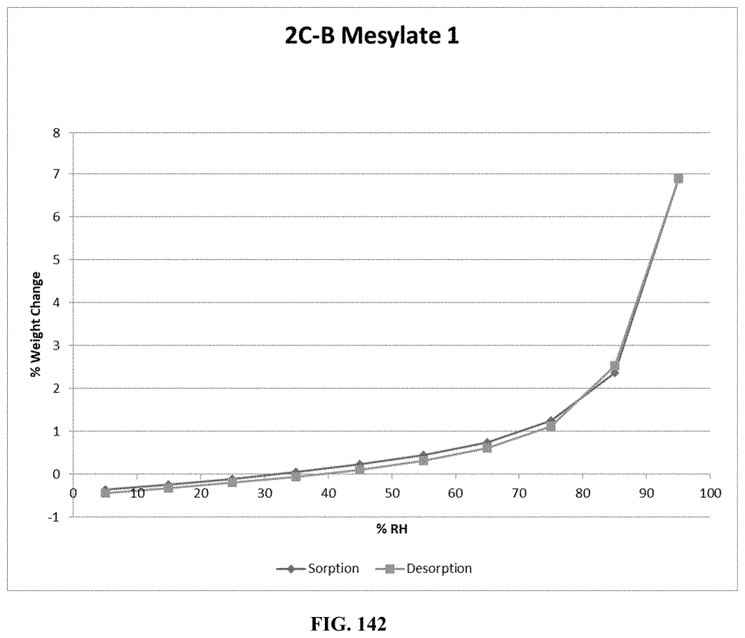

In some embodiments, 2C-B Mesylate has a DVS profile as provided in FIG. 142.

### 2C-B Mucate Salt

In some embodiments, the 2C-B Mucate salt is crystalline polymorphic characterized by two or more, or three XRPD signals selected from the group consisting of 15.5 °2θ, 24.9 °2θ, and 9.9 °2θ (±0.2 °2θ; ±0.1 °2θ; or ±0.0 °2θ; Cu Kα1 radiation).

In some embodiments, the 2C-B Mucate salt is a crystalline polymorph characterized by two or more, or three XRPD signals selected from the group consisting of 15.5 °2θ, 24.9 °2θ, 9.9 °2θ, and 20.2 °2θ (±0.2 °2θ; ±0.1 °2θ; or ±0.0 °2θ; Cu Kα1 radiation).

In some embodiments, the 2C-B Mucate salt is a crystalline polymorph characterized by two or more, or three XRPD signals selected from the group consisting of 15.5 °2θ, 24.9 °2θ, 9.9 °2θ, 20.2 °2θ, and 19.6 °2θ (±0.2 °2θ; ±0.1 °2θ; or ±0.0 °2θ; Cu Kα1 radiation).

In some embodiments, the 2C-B Mucate salt is a crystalline polymorph characterized by two or more, or three XRPD signals selected from the group consisting of 15.5 °2θ, 24.9 °2θ, 9.9 °2θ, 20.2 °2θ, 19.6 °2θ, 22.1 °2θ, 24.2 °2θ, 12 °2θ, 30.6 °2θ, and 34.4 °2θ (±0.2 °2θ; ±0.1 °2θ; or ±0.0 °2θ; Cu Kα1 radiation).

In some embodiments, the 2C-B Mucate salt is a crystalline polymorph characterized by two, three, four, five, six, seven, eight, nine, or ten XRPD signals as shown in Table 34.

In some embodiments, the 2C-B Mucate salt is a crystalline polymorph characterized by two or more, or three XRPD signals as shown in FIG. 23.

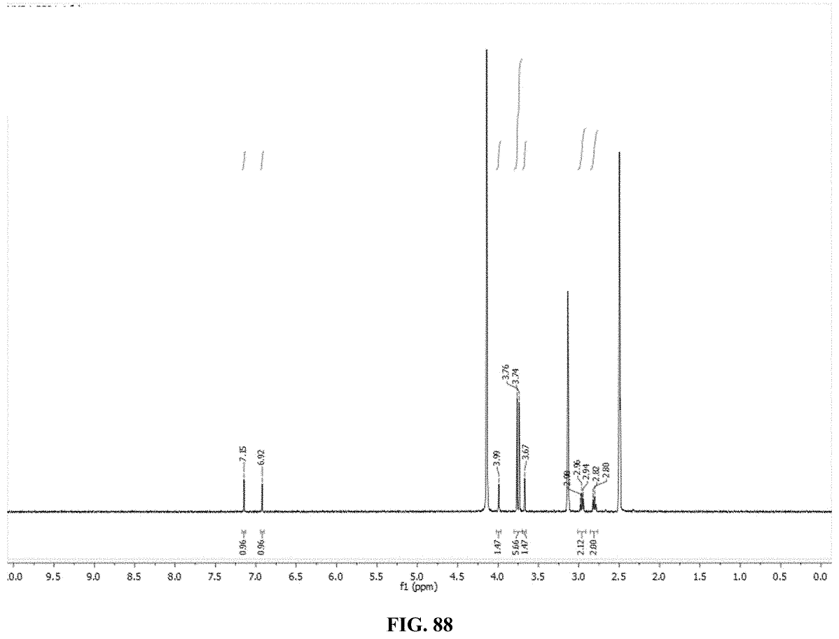

In some embodiments, 2C-B Mucate has an ¹H NMR spectra as provided in FIG. 88. In some embodiments, the 2C-B Mucate is a crystalline polymorph characterized by two or more, or three XRPD signals selected from the group consisting of 9.9 °2θ, 12.0 °2θ, and 15.5 °2θ (±0.2 °2θ; ±0.1 °2θ; or ±0.0 °2θ; Cu Kα1 radiation).

In some embodiments, the 2C-B Mucate is a crystalline polymorph characterized by two or more, or three XRPD signals selected from the group consisting of 9.9 °2θ, 12.0 °2θ, and 15.5 °2θ, 14.8 °2θ, and 19.6 °2θ (±0.2 °2θ; ±0.1 °2θ; or ±0.0 °2θ; Cu Kα1 radiation).

### 2C-B Succinate Salt

In some embodiments, the 2C-B succinate is crystalline polymorphic (Form 1 and Form 2 plus free succinic acid) characterized by two or more, or three XRPD signals selected from the group consisting of 17.6 °2θ, 25.9 °2θ, and 25.1 °2θ (±0.2 °2θ; ±0.1 °2θ; or ±0.0 °2θ; Cu Kα1 radiation).

In some embodiments, the 2C-B succinate is crystalline polymorphic (Form 1 and Form 2 plus free succinic acid) characterized by two or more, or three XRPD signals selected from the group consisting of 17.6 °2θ, 25.9 °2θ, 25.1 °2θ, and 16.7 °2θ (±0.2 °2θ; ±0.1 °2θ; or ±0.0 °2θ; Cu Kα1 radiation).

In some embodiments, the 2C-B succinate is crystalline polymorphic (Form 1 and Form 2 plus free succinic acid) characterized by two or more, or three XRPD signals selected from the group consisting of 17.6 °2θ, 25.9 °2θ, 25.1 °2θ, 16.7 °2θ, and 24.6 °2θ (±0.2 °2θ; ±0.1 °2θ; or ±0.0 °2θ; Cu Kα1 radiation).

In some embodiments, the 2C-B succinate is crystalline polymorphic (Form 1 and Form 2 plus free succinic acid) characterized by two or more, or three XRPD signals selected from the group consisting of 17.6 °2θ, 25.9 °2θ, 25.1 °2θ, 16.7 °2θ, 24.6 °2θ, 25.6 °2θ, 16.4 °2θ, 21.8 °2θ, and 21.3 °2θ (±0.2 °2θ; ±0.1 °2θ; or ±0.0 °2θ; Cu Kα1 radiation).

In some embodiments, the 2C-B succinate is crystalline polymorphic (Form 1 and Form 2 plus free succinic acid) characterized by two or more, or three XRPD signals selected from the group consisting of 8.2 °2θ, 11.1 °2θ, and 12.5 °2θ (±0.2 °2θ; ±0.1 °2θ; or ±0.0 °2θ; Cu Kα1 radiation).

In some embodiments, the 2C-B succinate is crystalline polymorphic (Form 1 and Form 2 plus free succinic acid) characterized by two or more, or three XRPD signals selected from the group consisting of 8.2 °2θ, 11.1 °2θ, 12.5 °2θ, 14.2 °2θ and 16.4 °2θ (±0.2 °2θ; ±0.1 °2θ; or ±0.0 °2θ; Cu Kα1 radiation).

In some embodiments, the 2C-B succinate is crystalline polymorphic (Form 1 and Form 2 plus free succinic acid) characterized by two, three, four, five, six, seven, eight, nine, or ten XRPD signals as shown in Table 35.

In some embodiments, the 2C-B succinate is crystalline polymorphic (Form 1 and Form 2 plus free succinic acid) characterized by two or more, or three XRPD signals as shown in FIG. 24.

In some embodiments, the 2C-B Succinate salt is crystalline polymorphic (Form 1) characterized by two or more, or three XRPD signals selected from the group consisting of 22.7 °2θ, 26 °2θ, and 17.6 °2θ (±0.2 °2θ; ±0.1 °2θ; or ±0.0 °2θ; Cu Kα1 radiation).

In some embodiments, the 2C-B Succinate salt is crystalline polymorphic (Form 1) characterized by two or more, or three XRPD signals selected from the group consisting of 22.7 °2θ, 26 °2θ, 17.6 °2θ, and 16.8 °2θ (±0.2 °2θ; ±0.1 °2θ; or ±0.0 °2θ; Cu Kα1 radiation).

In some embodiments, the 2C-B Succinate salt is crystalline polymorphic (Form 1) characterized by two or more, or three XRPD signals selected from the group consisting of 22.7 °2θ, 26 °2θ, 17.6 °2θ, 16.8 °2θ, and 23.9 °2θ (±0.2 °2θ; ±0.1 °2θ; or ±0.0 °2θ; Cu Kα1 radiation).

In some embodiments, the 2C-B Succinate salt is crystalline polymorphic (Form 1) characterized by two or more, or three XRPD signals selected from the group consisting of 22.7 °2θ, 26 °2θ, 17.6 °2θ, 16.8 °2θ, 23.9 °2θ, 25.1 °2θ, 7.6 °2θ, 30.4 °2θ, 21.3 °2θ, and 21.9 °2θ (±0.2 °2θ; ±0.1 °2θ; or ±0.0 °2θ; Cu Kα1 radiation).

In some embodiments, the 2C-B Succinate salt is crystalline polymorphic (Form 1) characterized by two, three, four, five, six, seven, eight, nine, or ten XRPD signals as shown in Table 43.

In some embodiments, the 2C-B Succinate salt is crystalline polymorphic (Form 1) characterized by two or more, or three XRPD signals as shown in FIG. 34.

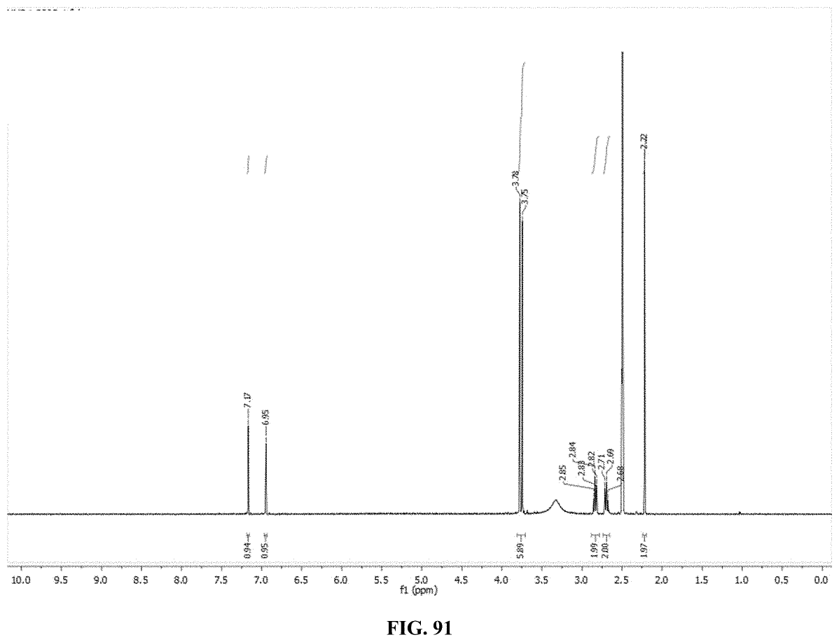

In some embodiments, 2C-B Succinate (Form 1) has an ¹H NMR spectra as provided in FIG. 91.

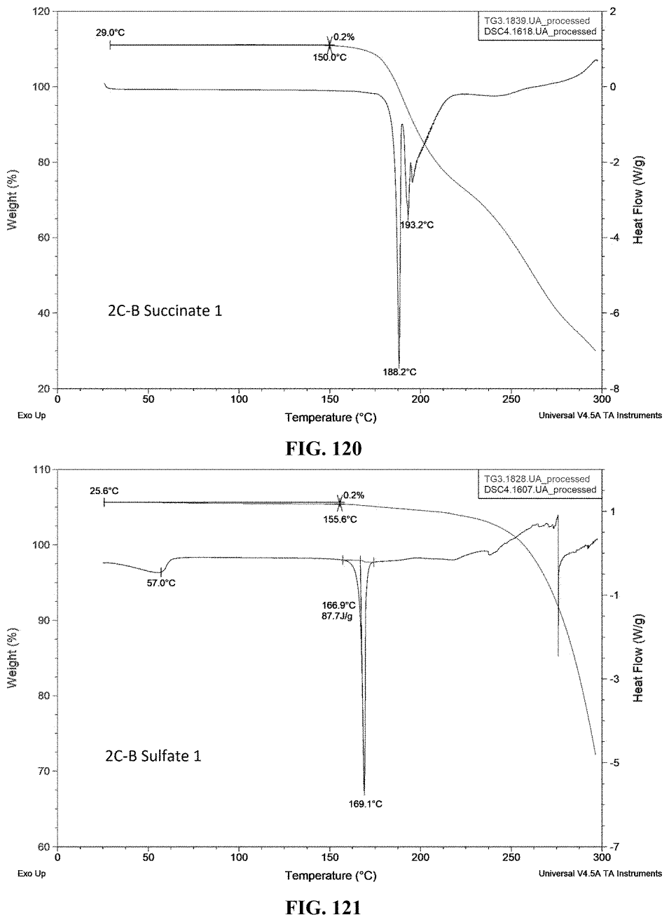

In some embodiments, 2C-B Succinate (Form 1) has TGA and DSC profiles as provided in FIG. 120.

In some embodiments, the 2C-B Succinate (Form 1) is a crystalline polymorph characterized by two or more, or three XRPD signals selected from the group consisting of 7.6 °2θ, 16.8 °2θ, and 17.6 °2θ (±0.2 °2θ; ±0.1 °2θ; or ±0.0 °2θ; Cu Kα1 radiation).

In some embodiments, the 2C-B Succinate (Form 1) is a crystalline polymorph characterized by two or more, or three XRPD signals selected from the group consisting of 7.6 °2θ, 16.8 °2θ, 17.6 °2θ, 22.7 °2θ, and 23.9 °2θ (±0.2 °2θ; ±0.1 °2θ; or ±0.0 °2θ; Cu Kα1 radiation).

### 2C-B Tartrate Salt

In some embodiments, the 2C-B Tartrate salt is crystalline polymorphic (Form 1) characterized by two or more, or three XRPD signals selected from the group consisting of 24.7 °2θ, 20.2 °2θ, and 20.5 °2θ (±0.2 °2θ; ±0.1 °2θ; or ±0.0 °2θ; Cu Kα1 radiation).

In some embodiments, the 2C-B Tartrate salt is crystalline polymorphic (Form 1) characterized by two or more, or three XRPD signals selected from the group consisting of 24.7 °2θ, 20.2 °2θ, 20.5 °2θ, and 23.2 °2θ (±0.2 °2θ; ±0.1 °2θ; or ±0.0 °2θ; Cu Kα1 radiation).

In some embodiments, the 2C-B Tartrate salt is crystalline polymorphic (Form 1) characterized by two or more, or three XRPD signals selected from the group consisting of 24.7 °2θ, 20.2 °2θ, 20.5 °2θ, 23.2 °2θ, and 27.2 °2θ (±0.2 °2θ; ±0.1 °2θ; or ±0.0 °2θ; Cu Kα1 radiation).

In some embodiments, the 2C-B Tartrate salt is crystalline polymorphic (Form 1) characterized by two or more, or three XRPD signals selected from the group consisting of 24.7 °2θ, 20.2 °2θ, 20.5 °2θ, 23.2 °2θ, 27.2 °2θ, 14.3 °2θ, 10.2 °2θ, 24.2 °2θ, 23.9 °2θ, and 12.8 °2θ (±0.2 °2θ; ±0.1 °2θ; or ±0.0 °2θ; Cu Kα1 radiation).

In some embodiments, the 2C-B Tartrate salt is crystalline polymorphic (Form 1) characterized by two or more, or three XRPD signals selected from the group consisting of 10.2 °2θ, 12.8 °2θ, and 14.2 °2θ (±0.2 °2θ; ±0.1 °2θ; or ±0.0 °2θ; Cu Kα1 radiation).

In some embodiments, the 2C-B Tartrate salt is crystalline polymorphic (Form 1) characterized by two or more, or three XRPD signals selected from the group consisting of 10.2 °2θ, 12.8 °2θ, 14.2 °2θ, 14.3 °2θ, and 20.2 °2θ (±0.2 °2θ; ±0.1 °2θ; or ±0.0 °2θ; Cu Kα1 radiation).

In some embodiments, the 2C-B Tartrate salt is crystalline polymorphic (Form 1) characterized by two, three, four, five, six, seven, eight, nine, or ten XRPD signals as shown in Table 36.

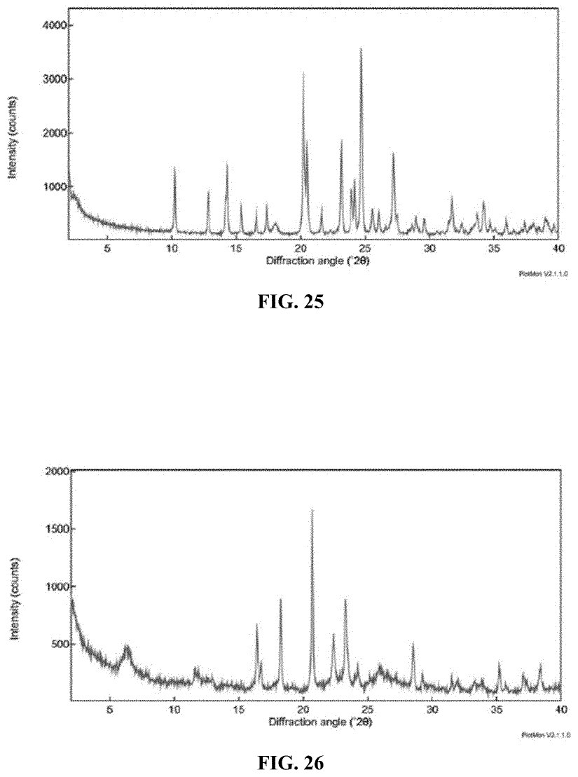

In some embodiments, the 2C-B Tartrate salt is crystalline polymorphic (Form 1) characterized by two or more, or three XRPD signals as shown in FIG. 25.

In some embodiments, the 2C-B Tartrate (Form 1) is a crystalline polymorph characterized by two or more, or three XRPD signals selected from the group consisting of 10.2 °2θ, 12.8 °2θ, and 14.3 °2θ (±0.2 °2θ; ±0.1 °2θ; or ±0.0 °2θ; Cu Kα1 radiation).

In some embodiments, the 2C-B Tartrate (Form 1) is a crystalline polymorph characterized by two or more, or three XRPD signals selected from the group consisting of 10.2 °2θ, 12.8 °2θ, 14.3 °2θ, 14.4 °2θ, and 20.3 °2θ (±0.2 °2θ; ±0.1 °2θ; or ±0.0 °2θ; Cu Kα1 radiation).

In some embodiments, the 2C-B Tartrate salt is crystalline polymorphic (Form 1) characterized by two or more, or three XRPD signals selected from the group consisting of 20.3 °2θ, 24.7 °2θ, and 27.2 °2θ (±0.2 °2θ; ±0.1 °2θ; or ±0.0 °2θ; Cu Kα1 radiation).

In some embodiments, the 2C-B Tartrate salt is crystalline polymorphic (Form 1) characterized by two or more, or three XRPD signals selected from the group consisting of 20.3 °2θ, 24.7 °2θ, 27.2 °2θ, and 14.4 °2θ (±0.2 °2θ; ±0.1 °2θ; or ±0.0 °2θ; Cu Kα1 radiation).

In some embodiments, the 2C-B Tartrate salt is crystalline polymorphic (Form 1) characterized by two or more, or three XRPD signals selected from the group consisting of 20.3 °2θ, 24.7 °2θ, 27.2 °2θ, 14.4 °2θ, and 14.2 °2θ (±0.2 °2θ; ±0.1 °2θ; or ±0.0 °2θ; Cu Kα1 radiation).

In some embodiments, the 2C-B Tartrate salt is crystalline polymorphic (Form 1) characterized by two or more, or three XRPD signals selected from the group consisting of 20.3 °2θ, 24.7 °2θ, 27.2 °2θ, 14.4 °2θ, 14.2 °2θ, 20.5 °2θ, 23.2 °2θ, 31.7 °2θ, 24 °2θ, and 33.7 °2θ (±0.2 °2θ; ±0.1 °2θ; or ±0.0 °2θ; Cu Kα1 radiation).

In some embodiments, the 2C-B Tartrate salt is crystalline polymorphic (Form 1) characterized by two, three, four, five, six, seven, eight, nine, or ten XRPD signals as shown in Table 70.

In some embodiments, the 2C-B Tartrate salt is crystalline polymorphic (Form 1) characterized by two or more, or three XRPD signals as shown in FIG. 64.

In some embodiments, the 2C-B Tartrate (Form 1) is a crystalline polymorph characterized by two or more, or three XRPD signals selected from the group consisting of 10.3 °2θ, 12.9 °2θ, and 14.2 °2θ (±0.2 °2θ; ±0.1 °2θ; or ±0.0 °2θ; Cu Kα1 radiation).

In some embodiments, the 2C-B Tartrate (Form 1) is a crystalline polymorph characterized by two or more, or three XRPD signals selected from the group consisting of 10.3 °2θ, 12.9 °2θ, 14.2 °2θ, 14.4 °2θ, and 20.3 °2θ (±0.2 °2θ; ±0.1 °2θ; or ±0.0 °2θ; Cu Kα1 radiation).

In some embodiments, the 2C-B Tartrate (Form 1) is a crystalline polymorph characterized by two or more, or three XRPD signals selected from the group consisting of 10.2 °2θ, 12.8 °2θ, and 14.3 °2θ (±0.2 °2θ; ±0.1 °2θ; or ±0.0 °2θ; Cu Kα1 radiation).

In some embodiments, the 2C-B Tartrate (Form 1) is a crystalline polymorph characterized by two or more, or three XRPD signals selected from the group consisting of 10.2 °2θ, 12.8 °2θ, 14.3 °2θ, 14.4, and 20.3 °2θ (±0.2 °2θ; ±0.1 °2θ; or ±0.0 °2θ; Cu Kα1 radiation).

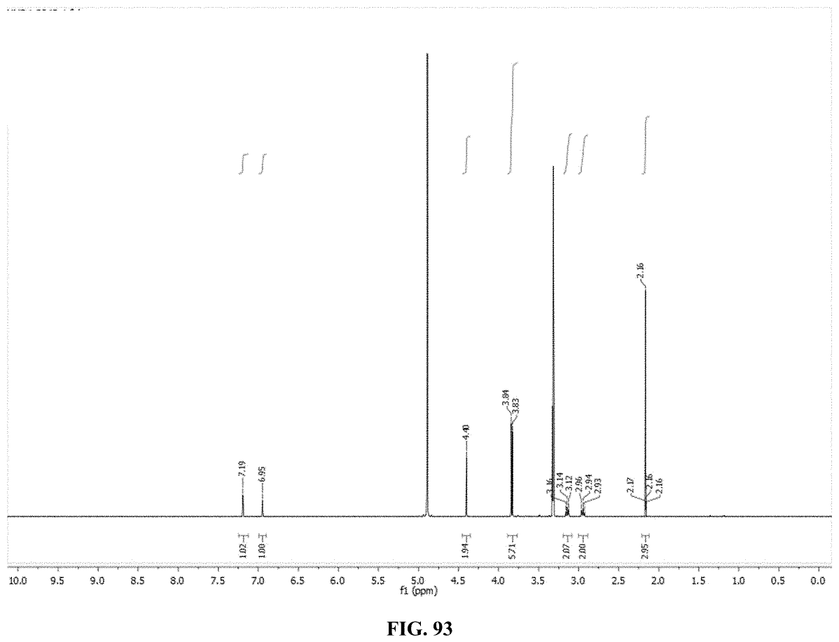

In some embodiments, 2C-B Tartrate (Form 1) has an ¹H NMR spectra as provided in FIG. 93.

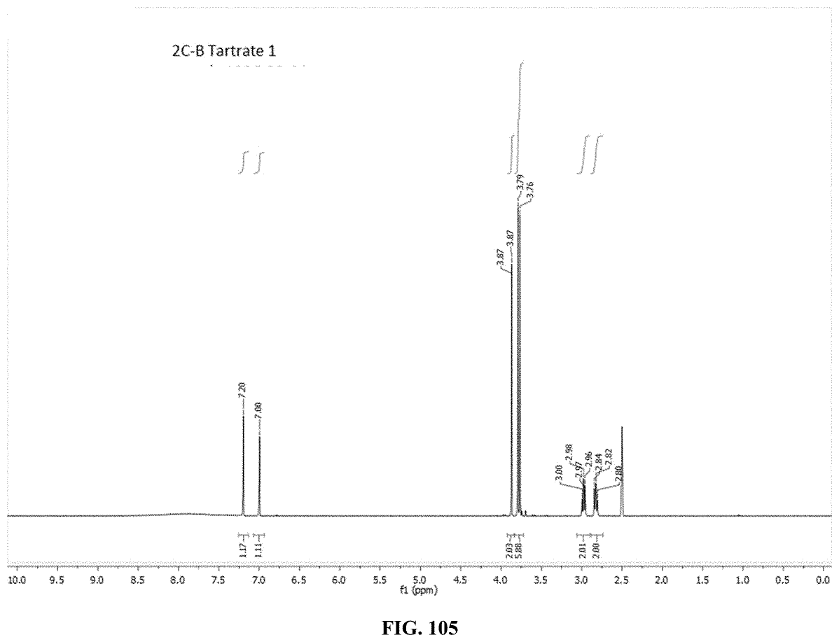

In some embodiments, 2C-B Tartrate (Form 1) has an ¹H NMR spectra as provided in FIG. 105.

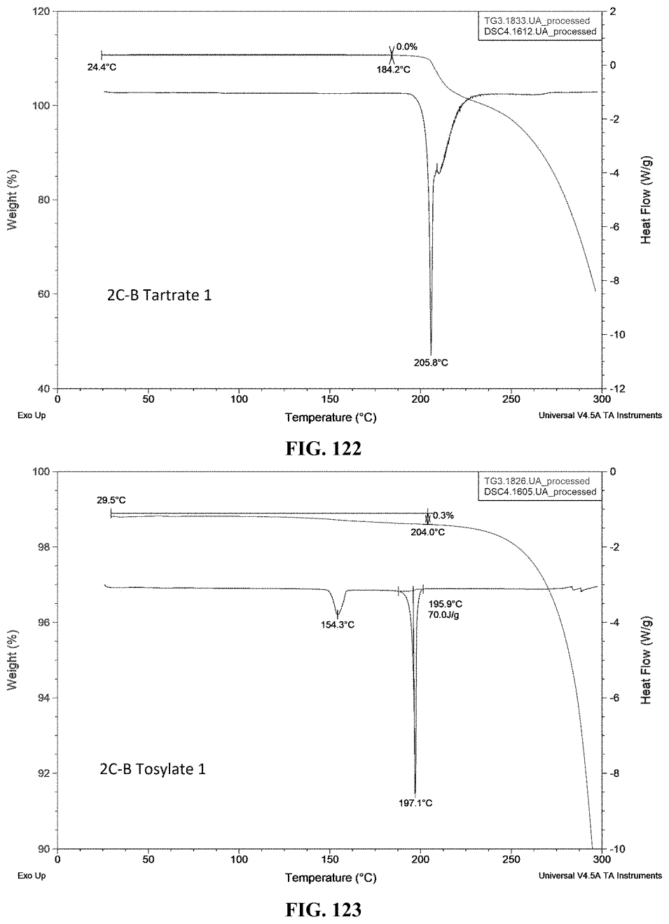

In some embodiments, 2C-B Tartrate (Form 1) has TGA and DSC profiles as provided in FIG. 122.

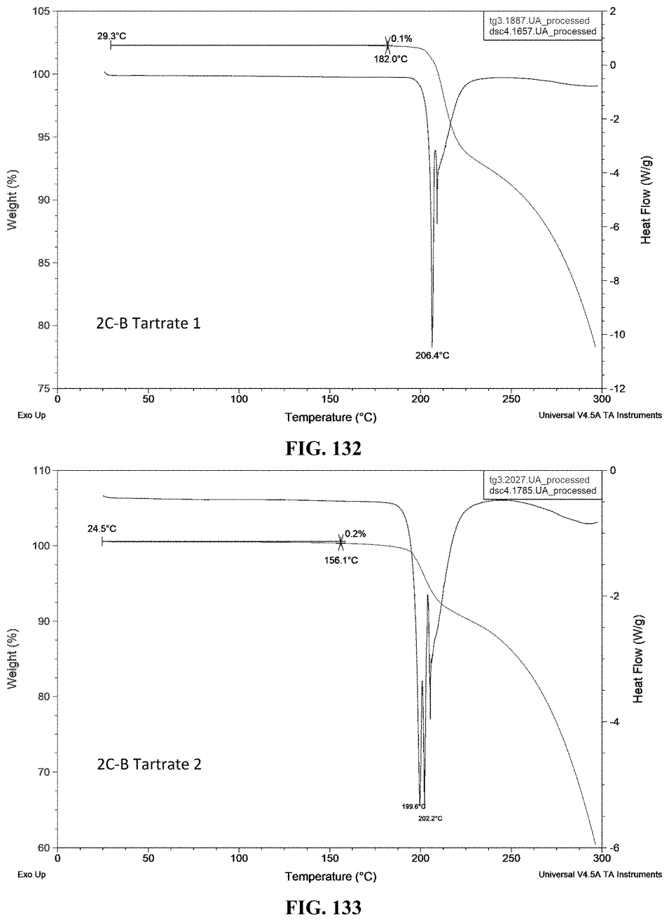

In some embodiments, 2C-B Tartrate (Form 1) has TGA and DSC profiles as provided in FIG. 132.

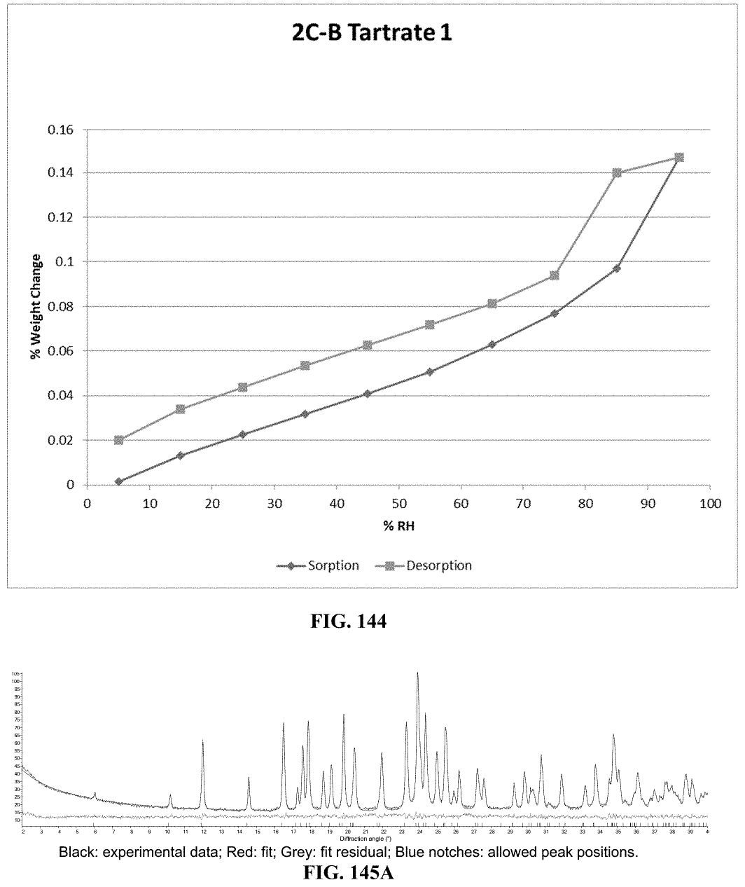

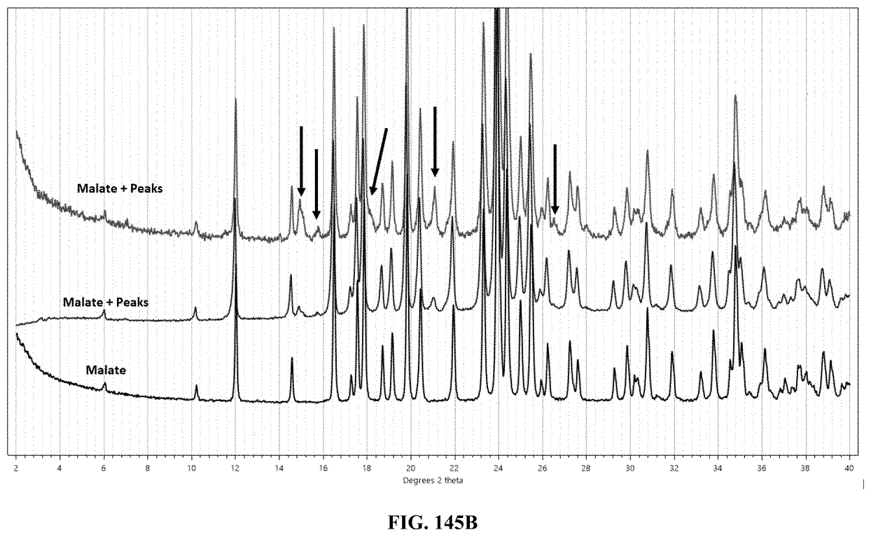

In some embodiments, 2C-B Tartrate (Form 1) has a DVS profile as provided in FIG. 144.

In some embodiments, the 2C-B Tartrate salt is crystalline polymorphic (Form 2) characterized by two or more, or three XRPD signals selected from the group consisting of 22.6 °2θ, 20.9 °2θ, and 11 °2θ (±0.2 °2θ; ±0.1 °2θ; or ±0.0 °2θ; Cu Kα1 radiation).

In some embodiments, the 2C-B Tartrate salt is crystalline polymorphic (Form 2) characterized by two or more, or three XRPD signals selected from the group consisting of 22.6 °2θ, 20.9 °2θ, 11 °2θ, and 28.8 °2θ (±0.2 °2θ; ±0.1 °2θ; or ±0.0 °2θ; Cu Kα1 radiation).

In some embodiments, the 2C-B Tartrate salt is crystalline polymorphic (Form 2) characterized by two or more, or three XRPD signals selected from the group consisting of 22.6 °2θ, 20.9 °2θ, 11 °2θ, 28.8 °2θ, and 12.4 °2θ (±0.2 °2θ; ±0.1 °2θ; or ±0.0 °2θ; Cu Kα1 radiation).

In some embodiments, the 2C-B Tartrate salt is crystalline polymorphic (Form 2) characterized by two or more, or three XRPD signals selected from the group consisting of 22.6 °2θ, 20.9 °2θ, 11 °2θ, 28.8 °2θ, 12.4 °2θ, 25.6 °2θ, 26.8 °2θ, 13.3 °2θ, 24.8 °2θ, and 20.6 °2θ (±0.2 °2θ; ±0.1 °2θ; or ±0.0 °2θ; Cu Kα1 radiation).

In some embodiments, the 2C-B Tartrate salt is crystalline polymorphic (Form 2) characterized by two, three, four, five, six, seven, eight, nine, or ten XRPD signals as shown in Table 71.

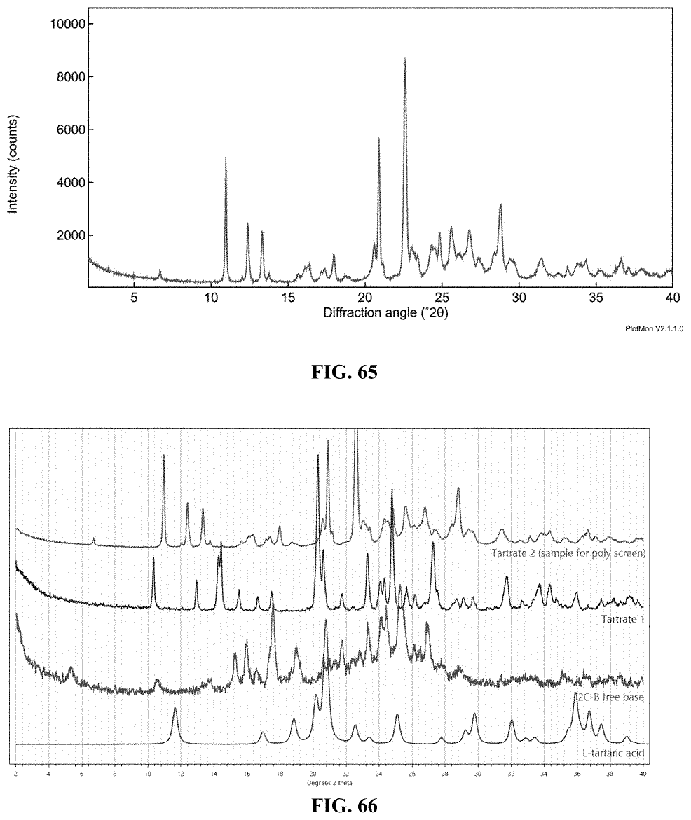

In some embodiments, the 2C-B Tartrate salt is crystalline polymorphic (Form 2) characterized by two or more, or three XRPD signals as shown in FIG. 65.

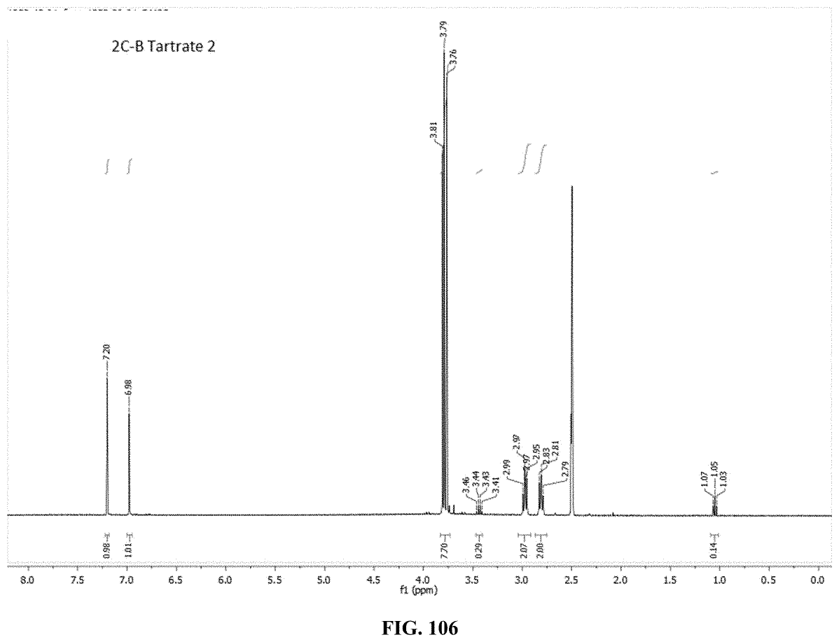

In some embodiments, 2C-B Tartrate (Form 2) has an ¹H NMR spectra as provided in FIG. 106.

In some embodiments, 2C-B Tartrate (Form 2) has TGA and DSC profiles as provided in FIG. 133.

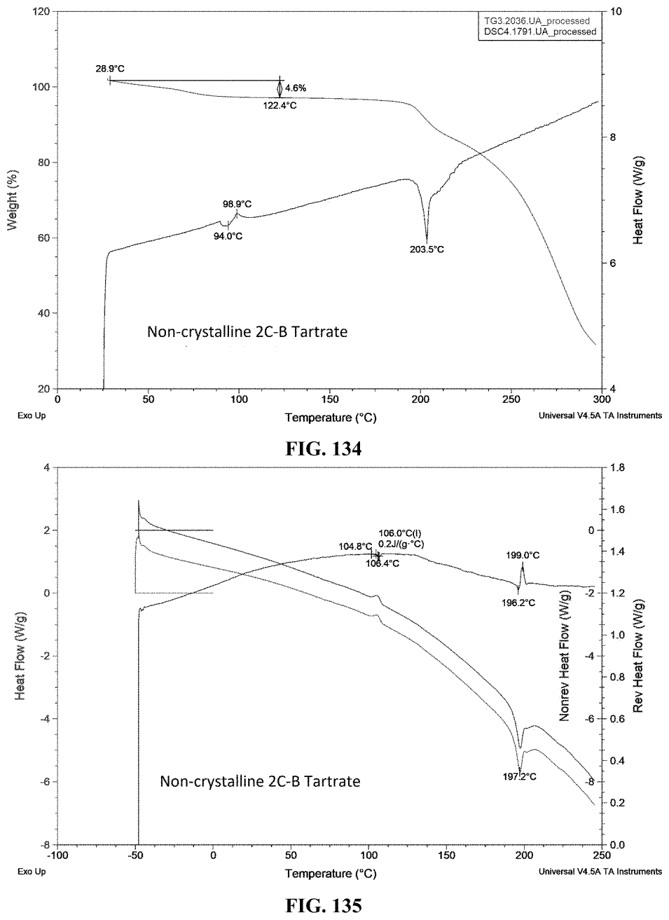

In some embodiments, non-crystalline 2C-B Tartrate has TGA and DSC profiles as provided in FIG. 134.

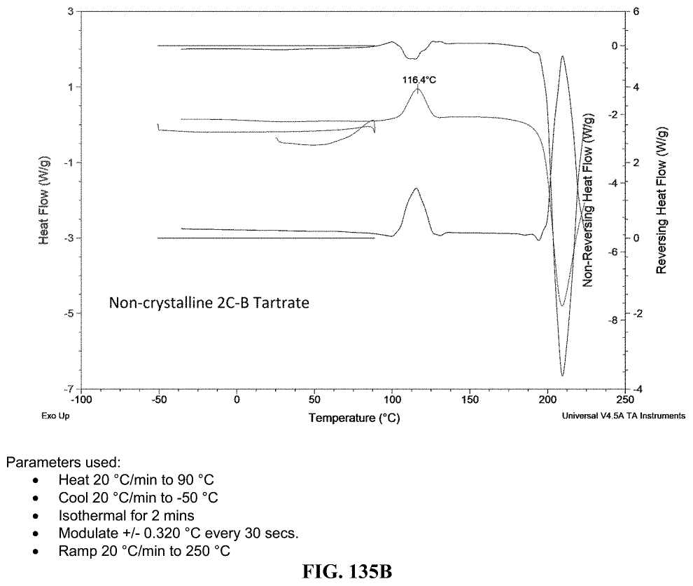

In some embodiments, non-crystalline 2C-B Tartrate has thermal profiles as provided in FIG. 135.

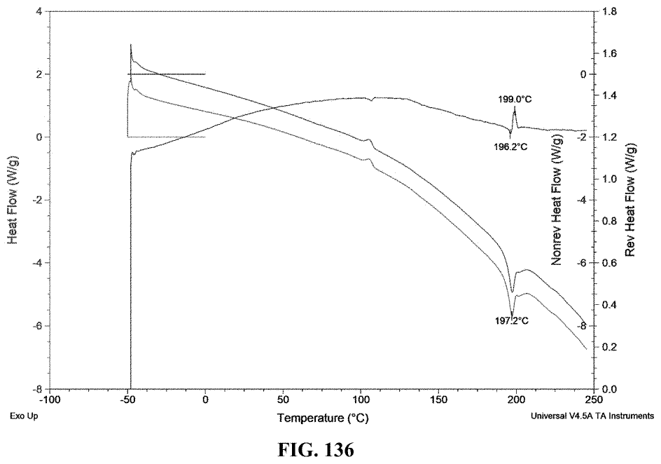

In some embodiments, non-crystalline 2C-B Tartrate has thermal profiles as provided in FIG. 136.

In some embodiments, the 2C-B Tartrate (Form 2) is a crystalline polymorph characterized by two or more, or three XRPD signals selected from the group consisting of 11.0 °2θ, 12.4 °2θ, and 13.3 °2θ (±0.2 °2θ; ±0.1 °2θ; or ±0.0 °2θ; Cu Kα1 radiation).

In some embodiments, the 2C-B Tartrate (Form 2) is a crystalline polymorph characterized by two or more, or three XRPD signals selected from the group consisting of 11.0 °2θ, 12.4 °2θ, 13.3 °2θ, 20.9 °2θ, and 22.6 °2θ (±0.2 °2θ; ±0.1 °2θ; or ±0.0 °2θ; Cu Kα1 radiation).

In some embodiments, the 2C-B tartrate salt (Form 1) possesses at least one of the following characteristics:

(a) exhibit high crystallinity,

(b) exhibit acceptable scale-up

(c) exhibit minimal water uptake over RH range,

(d) exhibit uncomplicated DSC data (one endothermic event observed), and

(e) acceptable solubility when compared to the other salts.

In some embodiments, the 2C-B tartrate salt (Form 1) possesses at least one of characteristic as provided in Table 86. In some embodiments, single crystal structure of 4-bromo-2,5-dimethoxyphenethylamine (2C-B) tartrate is anhydrous with 1:1 2C-B:tartaric acid stoichiometry and formula C₁₀H₁₅BrNO₂.C₄H₅O₆.

In some embodiments, single crystal structure of 4-bromo-2,5-dimethoxyphenethylamine (2C-B) tartrate (Form 1) has unit cell parameters as provided in Table 87A.

In some embodiments, the 2C-B tartrate salt (form 1) is prepared by the method provided in Example 5.

In one embodiment, the 2C-B tartrate salt is crystalline polymorphic form (Form 1) and is obtained by:

(i) adding L-tartaric acid to a solution of 2C-B free base in ethanol, resulting in precipitation,

(ii) adding more solvent to form a suspension,

(iii) slurring the suspension at room temperature for about 5 days, and

(iv) isolating solids via filtration and drying under vacuum at room temperature for about 1 day.

In another embodiment, the 2C-B tartrate salt is crystalline polymorphic form (Form 1) and is obtained by:

(i) adding L-tartaric acid to a solution of 2C-B free base in acetone, resulting in gel,

(ii) adding more solvent and heating at about 50° C. with stirring until the gel solidifies,

(iii) slurring the solidified gel at elevated temperature for about 4 days,

(iv) cooling to room temperature and stirring for about 6 days to form a product and mother liquor,

(v) centrifuging the product and mother liquor,

(vi) removing the mother liquor, and

(vii) isolating solids and drying.

In yet another the embodiment, 2C-B tartrate salt is crystalline polymorphic form (Form 2) and is obtained by:

(i) adding L-tartaric acid to a solution of 2C-B free base in ethanol to form a mixture,

(ii) sonicating the mixture to form a solid plug,

(iii) adding ethanol to form a suspension,

(iv) slurring the suspension at room temperature for about 4 days, and

(v) isolating solids via filtration and drying under vacuum at room temperature.

### 2C-B Tosylate Salt

In some embodiments, the 2C-B Tosylate salt is crystalline polymorphic (Form 1) characterized by two or more, or three XRPD signals selected from the group consisting of 20.7 °2θ, 23.3 °2θ, and 18.3 °2θ (±0.2 °2θ; ±0.1 °2θ; or ±0.0 °2θ; Cu Kα1 radiation).

In some embodiments, the 2C-B Tosylate salt is crystalline polymorphic (Form 1) In some embodiments, the 2C-B Tosylate salt is crystalline polymorphic (Form 1) characterized by two or more, or three XRPD signals selected from the group consisting of 20.7 °2θ, 23.3 °2θ, 18.3 °2θ, and 16.4 °2θ (±0.2 °2θ; ±0.1 °2θ; or ±0.0 °2θ; Cu Kα1 radiation).

In some embodiments, the 2C-B Tosylate salt is crystalline polymorphic (Form 1) characterized by two or more, or three XRPD signals selected from the group consisting of 20.7 °2θ, 23.3 °2θ, 18.3 °2θ, 16.4 °2θ, and 22.3 °2θ (±0.2 °2θ; ±0.1 °2θ; or ±0.0 °2θ; Cu Kα1 radiation).

In some embodiments, the 2C-B Tosylate salt is crystalline polymorphic (Form 1) characterized by two or more, or three XRPD signals selected from the group consisting of 20.7 °2θ, 23.3 °2θ, 18.3 °2θ, 16.4 °2θ, 22.3 °2θ, 6.2 °2θ, 28.5 °2θ, 16.8 °2θ, 24.2 °2θ, and 25.9 °2θ (±0.2 °2θ; ±0.1 °2θ; or ±0.0 °2θ; Cu Kα1 radiation).

In some embodiments, the 2C-B Tosylate salt is crystalline polymorphic (Form 1) characterized by two, three, four, five, six, seven, eight, nine, or ten XRPD signals as shown in Table 37.

In some embodiments, the 2C-B Tosylate salt is crystalline polymorphic (Form 1) characterized by two or more, or three XRPD signals as shown in FIG. 26.

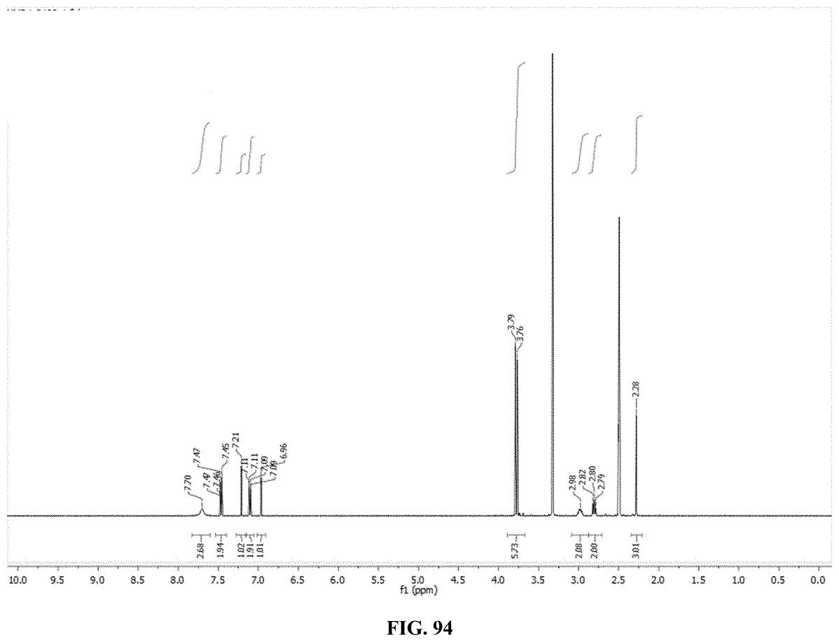

In some embodiments, 2C-B Tosylate (Form 1) has an ¹H NMR spectra as provided in FIG. 94.

In some embodiments, 2C-B Tosylate (Form 1) has TGA and DSC profiles as provided in FIG. 123.

In some embodiments, the 2C-B Tosylate (Form 1) is a crystalline polymorph characterized by two or more, or three XRPD signals selected from the group consisting of 16.4 °2θ, 18.3 °2θ, and 20.7 °2θ (±0.2 °2θ; ±0.1 °2θ; or ±0.0 °2θ; Cu Kα1 radiation).

In some embodiments, the 2C-B Tosylate (Form 1) is a crystalline polymorph characterized by two or more, or three XRPD signals selected from the group consisting of 16.4 °2θ, 18.3 °2θ, 20.7 °2θ, 22.3 °2θ, and 23.3 °2θ (±0.2 °2θ; ±0.1 °2θ; or ±0.0 °2θ; Cu Kα1 radiation).

In some embodiments, the 2C-B tosylate salt is crystalline polymorphic (Forms 1 and 2) characterized by two or more, or three XRPD signals selected from the group consisting of 23.3 °2θ, 16.4 °2θ, and 20.7 °2θ (±0.2 °2θ; ±0.1 °2θ; or ±0.0 °2θ; Cu Kα1 radiation).

In some embodiments, the 2C-B tosylate salt is crystalline polymorphic (Forms 1 and 2) characterized by two or more, or three XRPD signals selected from the group consisting of 23.3 °2θ, 16.4 °2θ, 20.7 °2θ, and 27.1 °2θ (±0.2 °2θ; ±0.1 °2θ; or ±0.0 °2θ; Cu Kα1 radiation).

In some embodiments, the 2C-B tosylate salt is crystalline polymorphic (Forms 1 and 2) characterized by two or more, or three XRPD signals selected from the group consisting of 23.3 °2θ, 16.4 °2θ, 20.7 °2θ, 27.1 °2θ, and 25.3 °2θ (±0.2 °2θ; ±0.1 °2θ; or ±0.0 °2θ; Cu Kα1 radiation).

In some embodiments, the 2C-B tosylate salt is crystalline polymorphic (Forms 1 and 2) characterized by two or more, or three XRPD signals selected from the group consisting of 23.3 °2θ, 16.4 °2θ, 20.7 °2θ, 27.1 °2θ, 25.3 °2θ, 24.8 °2θ, 28.5 °2θ, 16.8 °2θ, 12.6 °2θ, and 18 °2θ (±0.2 °2θ; ±0.1 °2θ; or ±0.0 °2θ; Cu Kα1 radiation).

In some embodiments, the 2C-B tosylate salt is crystalline polymorphic (Forms 1 and 2) characterized by two, three, four, five, six, seven, eight, nine, or ten XRPD signals as shown in Table 44.

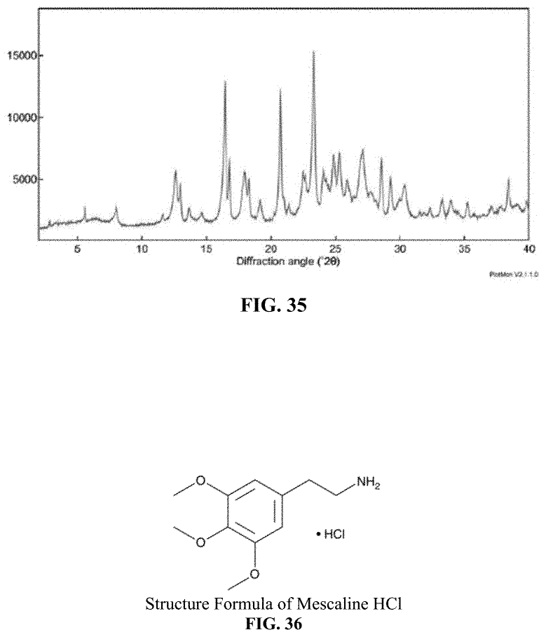

In some embodiments, the 2C-B tosylate salt is crystalline polymorphic (Forms 1 and 2) characterized by two or more, or three XRPD signals as shown in FIG. 35.

In some embodiments, the 2C-B tosylate (Form 2) is a crystalline polymorph characterized by two or more, or three XRPD signals selected from the group consisting of 8.0 °2θ, 12.6 °2θ, and 13.0 °2θ (±0.2 °2θ; ±0.1 °2θ; or ±0.0 °2θ; Cu Kα1 radiation).

In some embodiments, the 2C-B tosylate (Form 2) is a crystalline polymorph characterized by two or more, or three XRPD signals selected from the group consisting of 8.0 °2θ, 12.6 °2θ, 13.0 °2θ, 19.2 °2θ, and 20.7 °2θ (±0.2 °2θ; ±0.1 °2θ; or ±0.0 °2θ; Cu Kα1 radiation).

### 2C-B Aspartate Salt

In some embodiments, the 2C-B Aspartate salt is a crystalline polymorph characterized by two or more, or three XRPD signals selected from the group consisting of 24.2 °2θ, 16.5 °2θ, and 27.1 °2θ (±0.2 °2θ; ±0.1 °2θ; or ±0.0 °2θ; Cu Kα1 radiation).

In some embodiments, the 2C-B Aspartate salt is a crystalline polymorph characterized by two or more, or three XRPD signals selected from the group consisting of 24.2 °2θ, 16.5 °2θ, 27.1 °2θ, and 22.0 °2θ (±0.2 °2θ; ±0.1 °2θ; or ±0.0 °2θ; Cu Kα1 radiation).

In some embodiments, the 2C-B Aspartate salt is a crystalline polymorph characterized by two or more, or three XRPD signals selected from the group consisting of 24.2 °2θ, 16.5 °2θ, 27.1 °2θ, 22.0 °2θ, and 23.7 °2θ (±0.2 °2θ; ±0.1 °2θ; or ±0.0 °2θ; Cu Kα1 radiation).

In some embodiments, the 2C-B Aspartate salt is a crystalline polymorph characterized by two or more, or three XRPD signals selected from the group consisting of 24.2 °2θ, 16.5 °2θ, 27.1 °2θ, 22.0 °2θ, 23.7 °2θ, 8.2 °2θ, 23.5 °2θ, 4.1 °2θ, 13.7 °2θ and 29.1 °2θ (±0.2 °2θ; ±0.1 °2θ; or ±0.0 °2θ; Cu Kα1 radiation).

In some embodiments, the 2C-B Aspartate salt is crystalline polymorphic (aspartate+peaks) characterized by two, three, four, five, six, seven, eight, nine, or ten XRPD signals as shown in Table 45. The XRPD signals include the signals corresponding to 2C-B aspartate and other signals.

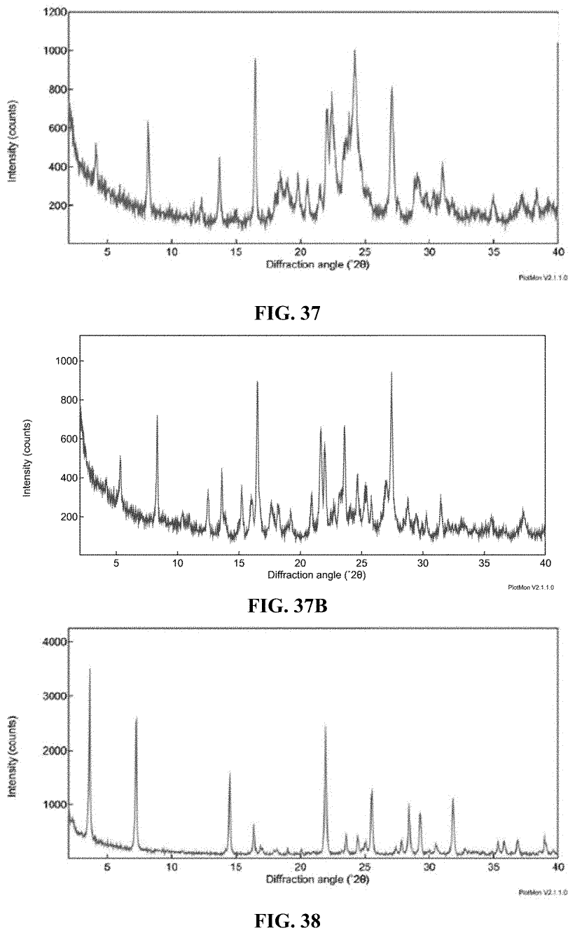

In some embodiments, the 2C-B Aspartate salt is crystalline polymorphic (aspartate+peaks) characterized by two or more, or three XRPD signals as shown in FIG. 37. The XRPD signals include the signals corresponding to 2C-B aspartate and other signals.

In some embodiments, the 2C-B Aspartate salt is crystalline polymorphic (aspartate+free base form 1 peaks) characterized by two or more, or three XRPD signals as shown in FIG. 37B.

In some embodiments, the 2C-B Aspartate is a crystalline polymorph characterized by two or more, or three XRPD signals selected from the group consisting of 8.2 °2θ, 13.7 °2θ, and 16.5 °2θ (±0.2 °2θ; ±0.1 °2θ; or ±0.0 °2θ; Cu Kα1 radiation). The XRPD signals include the signals corresponding to 2C-B aspartate and other signals.

In some embodiments, the 2C-B Aspartate is a crystalline polymorph characterized by two or more, or three XRPD signals selected from the group consisting of 8.2 °2θ, 13.7 °2θ, 16.5 °2θ, 4.1 °2θ, and 22.0 °2θ (±0.2 °2θ; ±0.1 °2θ; or ±0.0 °2θ; Cu Kα1 radiation). The XRPD signals include the signals corresponding to 2C-B aspartate and other signals.

### 2C-B Glutamate Salt

In some embodiments, the 2C-B Glutamate is crystalline polymorphic (PO) characterized by two or more, or three XRPD signals selected from the group consisting of 3.7 °2θ, 7.3 °2θ, and 21.9 °2θ (±0.2 °2θ; ±0.1 °2θ; or ±0.0 °2θ; Cu Kα1 radiation).

In some embodiments, the 2C-B Glutamate is crystalline polymorphic (PO) characterized by two or more, or three XRPD signals selected from the group consisting of 3.7 °2θ, 7.3 °2θ, 21.9 °2θ, and 14.5 °2θ (±0.2 °2θ; ±0.1 °2θ; or ±0.0 °2θ; Cu Kα1 radiation). In some embodiments, the 2C-B Glutamate is crystalline polymorphic (PO) characterized by two or more, or three XRPD signals selected from the group consisting of 3.7 °2θ, 7.3 °2θ, 21.9 °2θ, 14.5 °2θ, and 25.6 °2θ (±0.2 °2θ; ±0.1 °2θ; or ±0.0 °2θ; Cu Kα1 radiation).

In some embodiments, the 2C-B Glutamate is crystalline polymorphic (PO) characterized by two or more, or three XRPD signals selected from the group consisting of 3.7 °2θ, 7.3 °2θ, 21.9 °2θ, 14.5 °2θ, 25.6 °2θ, 31.8 °2θ, 28.4 °2θ, 29.3 °2θ, 16.4 °2θ, and 23.5 °2θ (±0.2 °2θ; ±0.1 °2θ; or ±0.0 °2θ; Cu Kα1 radiation).

In some embodiments, the 2C-B Glutamate is crystalline polymorphic (PO) characterized by two, three, four, five, six, seven, eight, nine, or ten XRPD signals as shown in Table 46.

In some embodiments, the 2C-B Glutamate is crystalline polymorphic (PO) characterized by two or more, or three XRPD signals as shown in FIG. 38.

In some embodiments, the 2C-B Glutamate (PO) is a crystalline polymorph characterized by two or more, or three XRPD signals selected from the group consisting of 21.9 °2θ, 3.7 °2θ, and 7.3 °2θ (±0.2 °2θ; ±0.1 °2θ; or ±0.0 °2θ; Cu Kα1 radiation).

In some embodiments, the 2C-B Glutamate (PO) is a crystalline polymorph characterized by two or more, or three XRPD signals selected from the group consisting of 21.9 °2θ, 3.7 °2θ, 7.3 °2θ, 14.5 °2θ, and 16.4 °2θ (±0.2 °2θ; ±0.1 °2θ; or ±0.0 °2θ; Cu Kα1 radiation).

In some embodiments, the 2C-B Glutamate is a crystalline polymorph characterized by two or more, or three XRPD signals selected from the group consisting of 22 °2θ, 25.5 °2θ, and 3.6 °2θ (±0.2 °2θ; ±0.1 °2θ; or ±0.0 °2θ; Cu Kα1 radiation).

In some embodiments, the 2C-B Glutamate is a crystalline polymorph characterized by two or more, or three XRPD signals selected from the group consisting of 22 °2θ, 25.5 °2θ, 3.6 °2θ, and 23.5 °2θ (±0.2 °2θ; ±0.1 °2θ; or ±0.0 °2θ; Cu Kα1 radiation).

In some embodiments, the 2C-B Glutamate is a crystalline polymorph characterized by two or more, or three XRPD signals selected from the group consisting of 22 °2θ, 25.5 °2θ, 3.6 °2θ, 23.5 °2θ, and 16.4 °2θ (±0.2 °2θ; ±0.1 °2θ; or ±0.0 °2θ; Cu Kα1 radiation).

In some embodiments, the 2C-B Glutamate is a crystalline polymorph characterized by two or more, or three XRPD signals selected from the group consisting of 22 °2θ, 25.5 °2θ, 3.6 °2θ, 23.5 °2θ, 16.4 °2θ, 24.5 °2θ, 16.9 °2θ, 17 °2θ, 24.8 °2θ, and 31.8 °2θ (±0.2 °2θ; ±0.1 °2θ; or 20±0.0 °2θ; Cu Kα1 radiation).

In some embodiments, the 2C-B Glutamate is a crystalline polymorph characterized by two, three, four, five, six, seven, eight, nine, or ten XRPD signals as shown in Table 64.

In some embodiments, the 2C-B Glutamate is a crystalline polymorph characterized by two or more, or three XRPD signals as shown in FIG. 58.

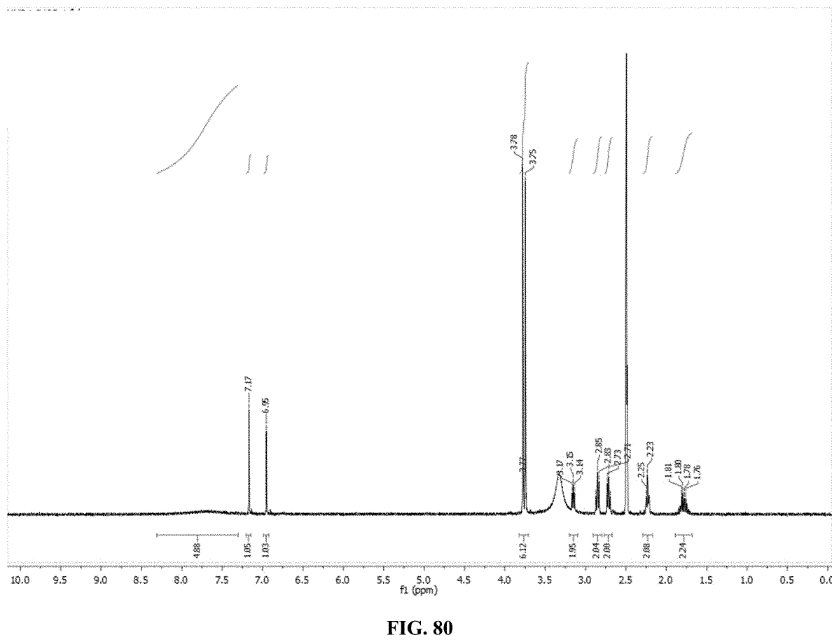

FIG. 80 depicts a proton NMR spectrum of 2C-B Glutamate.

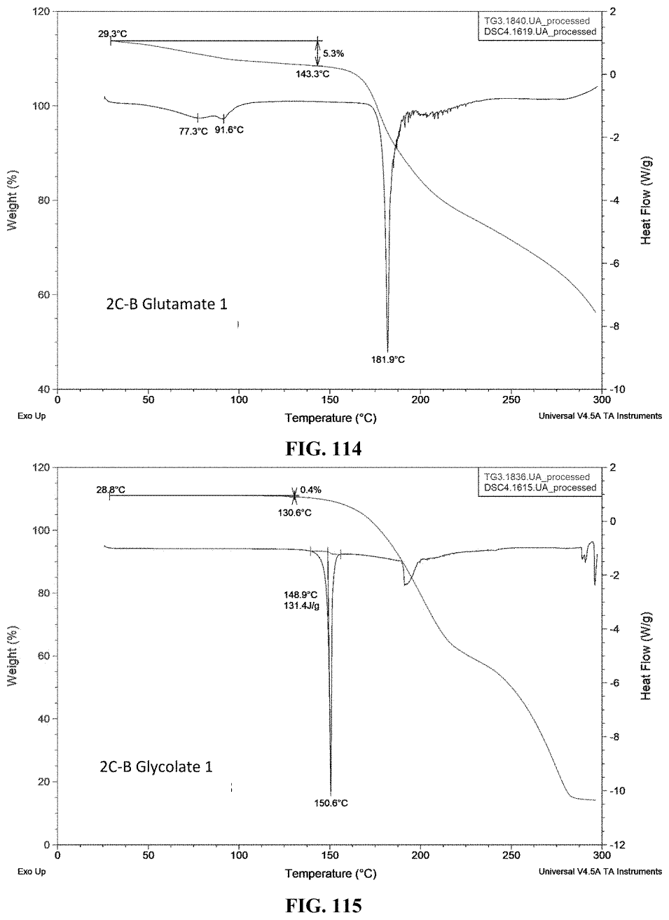

FIG. 114 depicts the TGA and DSC profiles of 2C-B Glutamate.

In some embodiments, 2C-B Glutamate has an ¹H NMR spectra as provided in FIG. 80.

In some embodiments, 2C-B Glutamate has TGA and DSC profiles as provided in FIG. 114.

In some embodiments, the 2C-B Glutamate is a crystalline polymorph characterized by two or more, or three XRPD signals selected from the group consisting of 3.6 °2θ, 7.3 °2θ, and 22.0 °2θ (±0.2 °2θ; ±0.1 °2θ; or ±0.0 °2θ; Cu Kα1 radiation).

In some embodiments, the 2C-B Glutamate is a crystalline polymorph characterized by two or more, or three XRPD signals selected from the group consisting of 3.6 °2θ, 7.3 °2θ, 22.0 °2θ, 14.6 °2θ, and 16.4 °2θ (±0.2 °2θ; ±0.1 °2θ; or ±0.0 °2θ; Cu Kα1 radiation).

### Solid Forms

Embodiments of 4-bromo-2,5-dimethoxyphenethylamine of the present disclosure are in a solid form. The solid form of the disclosed compounds may be a crystalline form or an amorphous form. In some embodiments, the solid form is a crystalline form.

A person of ordinary skill in the art understands that solid forms of 4-bromo-2,5-dimethoxyphenethylamine, such as crystalline forms including salt and non-salt crystalline forms of 4-bromo-2,5-dimethoxyphenethylamine, may exist in more than one crystal form. Such different forms are referred to as polymorphs.

In some embodiments, the solid form of 4-bromo-2,5-dimethoxyphenethylamine is a salt. And in certain embodiments, the solid form is a crystalline salt form of the compound.

In some embodiments, the disclosed compounds are particular polymorphs of 4-bromo-2,5-dimethoxyphenethylamine or 4-bromo-2,5-dimethoxyphenethylamine salts.

In some embodiments, the disclosed compound is a novel polymorph of 4-bromo-2,5-dimethoxyphenethylamine hydrochloride.

In some embodiments, the solid form of 4-bromo-2,5-dimethoxyphenethylamine or 4-bromo-2,5-dimethoxyphenethylamine hydrochloride disclosed herein is selected to be a crystalline form, such as a particular polymorph of a crystalline form of 4-bromo-2,5-dimethoxyphenethylamine or 4-bromo-2,5-dimethoxyphenethylamine hydrochloride, that provides one or more desired properties. In one embodiment, the crystalline form offers advantages over the amorphous form of the molecule. In another embodiment, the disclosed polymorph offers improved properties as compared to another polymorph of 4-bromo-2,5-dimethoxyphenethylamine. The 4-bromo-2,5-dimethoxyphenethylamine may be a salt or a free base compound. The one or more desired properties may include, but are not limited to, physical properties, including but not limited to, melting point, glass transition temperature, flowability, and/or stability, such as thermal stability, mechanical stability, shelf life, stability against polymorphic transition, etc.; chemical properties, such as, but not limited to, hygroscopic properties, solubility in water and/or organic solvents, reactivity, compatibility with excipients and/or delivery vehicles; and/or pharmacokinetic properties, such as, but not limited to, bioavailability, absorption, distribution, metabolism, excretion, toxicity including cytotoxicity, dissolution rate, and/or half-life.

The desired polymorph may be produced by techniques known to persons of ordinary skill in the art. Such techniques include, but are not limited to, crystallization in particular solvents and/or at particular temperatures, supersaturation, using a precipitation agent, such as a salt, glycol, alcohol, etc., co-crystallization, lyophilization, spray drying, freeze drying, and/or complexing with an inert agent.

Techniques to identify a particular solid form of 4-bromo-2,5-dimethoxyphenethylamine and also are known to persons of ordinary skill in the art, and include, but are not limited to, X-ray crystallography, X-ray diffraction, electron crystallography, powder diffraction, including X-ray, neutron, or electron diffraction, X-ray fiber diffraction, small-angle X-ray scattering, and/or melting point.

### Pharmaceutical Compositions and Formulations

In some embodiments, the present disclosure provides a pharmaceutical composition comprising one or more of the salts and solid forms of 4-bromo-2,5-dimethoxyphenethylamine, and a pharmaceutically acceptable excipient. Such compositions are suitable for administration to a subject, such as a human subject.

The presently disclosed pharmaceutical compositions can be prepared in a wide variety of oral, parenteral and topical dosage forms. Oral preparations include tablets, pills, powder, capsules, lozenges, cachets, slurries, suspensions, etc., suitable for ingestion by the patient. The compositions of the present invention can also be administered by injection, that is, intravenously, intramuscularly, intracutaneously, subcutaneously, intraduodenally, or intraperitoneally. Also, the compositions described herein can be administered by inhalation, for example, intranasally. Additionally, the compositions of the present invention can be administered transdermally. The compositions of this invention can also be administered by intraocular, intravaginal, and intrarectal routes including suppositories, insufflation, powders and aerosol formulations (for examples of steroid inhalants, see Rohatagi, J. Clin. Pharmacol. 35:1187-1193, 1995; Tjwa, Ann. Allergy Asthma Immunol. 75:107-111, 1995). Accordingly, the present disclosure also provides pharmaceutical compositions including a pharmaceutically acceptable carrier or excipient and the solid form of 4-bromo-2,5-dimethoxyphenethylamine of the present disclosure.

For preparing pharmaceutical compositions from the compounds disclosed herein, pharmaceutically acceptable carriers can be either solid or liquid. Solid form preparations include powders, tablets, pills, capsules, cachets, suppositories, and dispersible granules. A solid carrier can be one or more substances, which may also act as diluents, flavoring agents, binders, preservatives, tablet disintegrating agents, or an encapsulating material. Details on techniques for formulation and administration are well described in the scientific and patent literature, see, e.g., the latest edition of Remington's Pharmaceutical Sciences, Mack Publishing Co, Easton Pa. (“Remington's”).

In powders, the carrier is a finely divided solid, which is in a mixture with the finely divided active component. In tablets, the active component is mixed with the carrier having the necessary binding properties in suitable proportions and compacted in the shape and size desired. The powders and tablets preferably contain from 5% to 70% or 10% to 70% of the compounds of the present disclosure.

Suitable solid excipients include, but are not limited to, magnesium carbonate; magnesium stearate; talc; pectin; dextrin; starch; tragacanth; a low melting wax; cocoa butter; carbohydrates; sugars including, but not limited to, lactose, sucrose, mannitol, or sorbitol, starch from corn, wheat, rice, potato, or other plants; cellulose such as methyl cellulose, hydroxypropylmethylcellulose, or sodium carboxymethylcellulose; and gums including arabic and tragacanth; as well as proteins including, but not limited to, gelatin and collagen.

If desired, disintegrating or solubilizing agents may be added, such as the cross-linked polyvinyl pyrrolidone, agar, alginic acid, or a salt thereof, such as sodium alginate.

For preparing suppositories, a low melting wax, such as a mixture of fatty acid glycerides or cocoa butter, is first melted and the compounds of the present invention are dispersed homogeneously therein, as by stirring. The molten homogeneous mixture is then poured into convenient sized molds, allowed to cool, and thereby to solidify.

Liquid form preparations include solutions and suspensions, for example, water or water/propylene glycol suspensions.

Aqueous suspensions suitable for oral use can be made by dispersing the finely divided active component in water with viscous material, such as natural or synthetic gums, resins, methylcellulose, sodium carboxymethylcellulose, hydroxypropylmethylcellulose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethylene oxycetanol), a condensation product of ethylene oxide with a partial ester derived from a fatty acid and a hexitol (e.g., polyoxyethylene sorbitol mono-oleate), or a condensation product of ethylene oxide with a partial ester derived from fatty acid and a hexitol anhydride (e.g., polyoxyethylene sorbitan mono-oleate). The aqueous suspension can also contain one or more preservatives such as ethyl or n-propyl p-hydroxybenzoate, one or more coloring agents, one or more flavoring agents and one or more sweetening agents, such as sucrose, aspartame or saccharin. Formulations can be adjusted for osmolarity.

Also included are solid form preparations, which are intended to be converted, shortly before use, to liquid form preparations for oral administration. Such liquid forms include suspensions. These preparations may contain, in addition to the active component, colorants, flavors, stabilizers, buffers, artificial and natural sweeteners, dispersants, thickeners, solubilizing agents, and the like.

Oil suspensions can be formulated by suspending the compound of the present invention in a vegetable oil, such as arachis oil, olive oil, sesame oil or coconut oil, or in a mineral oil such as liquid paraffin; or a mixture of these. The oil suspensions can contain a thickening agent, such as beeswax, hard paraffin or cetyl alcohol. Sweetening agents can be added to provide a palatable oral preparation, such as glycerol, sorbitol or sucrose. These formulations can be preserved by the addition of an antioxidant such as ascorbic acid. As an example of an injectable oil vehicle, see Minto, J. Pharmacol. Exp. Ther. 281:93-102, 1997. The pharmaceutical formulations of the invention can also be in the form of oil-in-water emulsions. The oily phase can be a vegetable oil or a mineral oil, described above, or a mixture of these. Suitable emulsifying agents include naturally-occurring gums, such as gum acacia and gum tragacanth, naturally occurring phosphatides, such as soybean lecithin, esters or partial esters derived from fatty acids and hexitol anhydrides, such as sorbitan mono-oleate, and condensation products of these partial esters with ethylene oxide, such as polyoxyethylene sorbitan mono-oleate. The emulsion can also contain sweetening agents and flavoring agents, as in the formulation of syrups and elixirs. Such formulations can also contain a demulcent, a preservative, or a coloring agent.

The compositions of the present disclosure can also be delivered as microspheres for slow release in the body. For example, microspheres can be formulated for administration via intradermal injection of drug-containing microspheres, which slowly release subcutaneously (see Rao, J. Biomater Sci. Polym. Ed. 7:623-645, 1995; as biodegradable and injectable gel formulations (see, e.g., Gao Pharm. Res. 12:857-863, 1995); or, as microspheres for oral administration (see, e.g., Eyles, J. Pharm. Pharmacol. 49:669-674, 1997). Both transdermal and intradermal routes afford constant delivery for weeks or months.

In some embodiments, the pharmaceutical compositions of the present disclosure can be formulated for parenteral administration, such as intravenous (IV) administration or administration into a body cavity or lumen of an organ. The formulations for administration will commonly comprise a solution or suspension of the compositions of the present disclosure dissolved or suspended in a pharmaceutically acceptable carrier. Among the acceptable vehicles and solvents that can be employed are water and Ringer's solution, an isotonic sodium chloride. In addition, sterile fixed oils can conventionally be employed as a solvent or suspending medium. For this purpose, any bland fixed oil can be employed including synthetic mono- or diglycerides. In addition, fatty acids such as oleic acid can likewise be used in the preparation of injectables. These solutions or suspensions are sterile and generally free of undesirable matter. These formulations may be sterilized by conventional, well known sterilization techniques. The formulations may contain pharmaceutically acceptable auxiliary substances as required to approximate physiological conditions such as pH adjusting and buffering agents, toxicity adjusting agents, e.g., sodium acetate, sodium chloride, potassium chloride, calcium chloride, sodium lactate and the like. The concentration of the compositions of the present disclosure in these formulations can vary widely, and will be selected primarily based on fluid volumes, viscosities, body weight, and the like, in accordance with the particular mode of administration selected and the patient's needs. For IV administration, the formulation can be a sterile injectable preparation, such as a sterile injectable aqueous or oleaginous suspension. This suspension can be formulated according to the known art using those suitable dispersing or wetting agents and suspending agents. The sterile injectable preparation can also be a sterile injectable solution or suspension in a nontoxic parenterally-acceptable diluent or solvent, such as a solution of 1,3-butanediol.

In some embodiments, the formulations of the compositions of the present disclosure can be delivered by the use of liposomes which fuse with the cellular membrane or are endocytosed, for example, by employing ligands attached to the liposome, or attached directly to the oligonucleotide, that bind to surface membrane protein receptors of the cell resulting in endocytosis. By using liposomes, particularly where the liposome surface carries ligands specific for target cells, or are otherwise preferentially directed to a specific organ, one can focus the delivery of the compositions of the present invention into the target cells in vivo. (See, e.g., Al-Muhammed, J. Microencapsul. 13:293-306, 1996; Chonn, Curr. Opin. Biotechnol. 6:698-708, 1995; Ostro, Am. J. Hosp. Pharm. 46:1576-1587, 1989).

### Administration:

The compositions of the present disclosure can be administered by any suitable means, including oral, parenteral and topical methods. Transdermal administration methods, by a topical route, can be formulated as applicator sticks, suspensions, creams, ointments, pastes, jellies, paints, powders, and aerosols.

The pharmaceutical preparation is preferably in unit dosage form. In such form the preparation is subdivided into unit doses containing appropriate quantities of the compounds of the present invention. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

The compounds of the present invention can be present in any suitable amount, and can depend on various factors including, but not limited to, weight and age of the subject, state of the disease, and the like as is known to those of ordinary skill in the art.

Suitable dosage ranges for the compounds disclosed herein include from about 0.1 mg to about 10,000 mg, or about 1 mg to about 1000 mg, or about 10 mg to about 750 mg, or about 25 mg to about 500 mg, or about 50 mg to about 250 mg. Suitable dosages for the compound of the present invention include about 1 mg, 5, 10, 20, 30, 40, 50, 60, 70, 80, 90, 100, 200, 300, 400, 500, 600, 700, 800, 900 or 1000 mg.

The compounds disclosed herein can be administered at any suitable frequency, interval and duration.

The compounds of the present invention can be co-administered with a second active agent.

The compounds disclosed herein can be administered at any suitable frequency, interval and duration. For example, the compounds can be administered once an hour, or two, three or more times an hour, once a day, or two, three, or more times per day, or once every 2, 3, 4, 5, 6, or 7 days, so as to provide the preferred dosage level. When the compound of the present invention is administered more than once a day, representative intervals include 5, 10, 15, 20, 30, 45 and 60 minutes, as well as 1, 2, 4, 6, 8, 10, 12, 16, 20, and 24 hours. The compound of the present invention can be administered once, twice, or three or more times, for an hour, for 1 to 6 hours, for 1 to 12 hours, for 1 to 24 hours, for 6 to 12 hours, for 12 to 24 hours, for a single day, for 1 to 7 days, for a single week, for 1 to 4 weeks, for a month, for 1 to 12 months, for a year or more, or even indefinitely.

The composition can also contain other compatible therapeutic agents. The compounds described herein can be used in combination with one another, with other active agents known to be useful in modulating a glucocorticoid receptor, or with adjunctive agents that may not be effective alone, but may contribute to the efficacy of the active agent.

The compounds of the present invention can be co-administered with a second active agent. Co-administration includes administering the compound of the present invention and active agent within 0.5, 1, 2, 4, 6, 8, 10, 12, 16, 20, or 24 hours of each other. Co-administration also includes administering the compound of the present invention and active agent simultaneously, approximately simultaneously (e.g., within about 1, 5, 10, 15, 20, or 30 minutes of each other), or sequentially in any order. Moreover, the compound of the present disclosure and the active agent can each be administered once a day, or two, three, or more times per day so as to provide the preferred dosage level per day.

The compounds of the present invention can be co-administered with a second active agent.

In some embodiments, co-administration can be accomplished by co-formulation, such as by preparing a single pharmaceutical composition including both the compound of the present disclosure and a second active agent. In other embodiments, the compound of the present disclosure and the second active agent can be formulated separately.

The disclosed compounds and the second active agent can be present in the compositions of the present disclosure in any suitable weight ratio, such as from about 1:100 to about 100:1 (w/w), or about 1:50 to about 50:1, or about 1:25 to about 25:1, or about 1:10 to about 10:1, or about 1:5 to about 5:1 (w/w). The compound of the present disclosure and the second active agent can be present in any suitable weight ratio, such as about 1:100 (w/w), 1:50, 1:25, 1:10, 1:5, 1:4, 1:3, 1:2, 1:1, 2:1, 3:1, 4:1, 5:1, 10:1, 25:1, 50:1 or 100:1 (w/w). Other dosages and dosage ratios of the compound of the present disclosure and the active agent are suitable in the compositions and methods disclosed herein.

In some embodiments, the 2C-B form is provided in a range of about 1 milligram (mg) to about 50 mg, such as from about 20 mg to about 40 mg, or from about 0.5 mg to about 30 mg. In some embodiments, the 2C-B form is provided from about 1 mg, about 15 mg, about 20 mg, about 25 mg, about 30 mg, about 35 mg to about 40 mg. In some embodiments, the amount of the 2C-B form is provided on a 2C-B basis.

In some embodiments, the 2C-B.HCl is provided in a range about 1 milligram (mg) to about 50 mg, such as from about 20 mg to about 40 mg, or from about 0.5 mg to about 30 mg. In some embodiments, the 2C-B.HCl is provided from about 1 mg, about 15 mg, about 20 mg, about 25 mg, about 30 mg, about 35 mg to about 40 mg. In some embodiments, the amount of 2C-B HCl is provided on a 2C-B basis.

In some embodiments, the 2C-B forms disclosed herein, including those described in Table 3 and Table 18, are provided in a range of about 1 milligram (mg) to about 50 mg, such as from about 20 mg to about 40 mg, or from about 0.5 mg to about 30 mg. In some embodiments, the 2C-B form is provided from about 1 mg, about 15 mg, about 20 mg, about 25 mg, about 30 mg, about 35 mg to about 40 mg. In some embodiments, the amount of the 2C-B form is provided on a 2C-B basis.

### Methods of Treatment

The salts and solid forms of 4-bromo-2,5-dimethoxyphenethylamine can be used for increasing neuronal plasticity. The compounds of the present disclosure can also be used to treat any brain disease. The compounds of the present disclosure can also be used for increasing at least one of translation, transcription or secretion of neurotrophic factors.

In some embodiments, the methods described herein are for treating a disease or disorder that is a brain disease or disorder. In some embodiments, the methods described herein are for increasing at least one of translation, transcription or secretion of neurotrophic factors. In some embodiments, the compositions provided herein have, for example, anti-addictive properties, antidepressant properties, anxiolytic properties, or a combination thereof. In some embodiments, the neurological disease is a neuropsychiatric disease. In some embodiments, the brain disorder is a neuropsychiatric disease. In some embodiments, the methods described herein are for treating a disease or disorder that is a neuropsychiatric disease. In some embodiments, the neuropsychiatric disease is a mood or anxiety disorder. In some embodiments, brain disorders include, for example, migraine, cluster headache, post-traumatic stress disorder (PTSD), anxiety, depression, panic disorder, suicidality, schizophrenia, and addiction (e.g., substance abuse disorder). In some embodiments, brain disorders include, for example, migraines, addiction (e.g., substance use disorder for example alcohol abuse, opiate addition, or abuse), depression, and anxiety. In some embodiments, the neurological disease is a migraine or cluster headache. In some embodiments, the brain disease or disorder is a neurodegenerative disorder, Alzheimer's disease or Parkinson's disease. In some embodiments, the brain disease or disorder is psychological disorder, depression, addiction, anxiety, or a post-traumatic stress disorder. In some embodiments, the brain disorder is depression. In some embodiments, the brain disorder is addiction. In some embodiments, the brain disorder is treatment resistant depression, suicidal ideation, major depressive disorder, bipolar disorder, schizophrenia, stroke, traumatic brain injury or substance use disorder. In some embodiments, the brain disorder is treatment resistant depression, suicidal ideation, major depressive disorder, bipolar disorder, schizophrenia, or substance use disorder. In some embodiments, the brain disorder is stroke or traumatic brain injury. In some embodiments, the brain disorder is treatment resistant depression, suicidal ideation, major depressive disorder, bipolar disorder, or substance use disorder. In some embodiments, the brain disorder is schizophrenia. In some embodiments, the brain disorder is alcohol use disorder.

In some embodiments, the methods described herein are for treating a disease or disorder that is a neurological disease. For example, a compound provided herein can exhibit, anti-addictive properties, antidepressant properties, anxiolytic properties, or a combination thereof. In some embodiments, the neurological disease is a neuropsychiatric disease. In some embodiments, the neuropsychiatric disease is a mood or anxiety disorder. In some embodiments, the neurological disease is a migraine, headaches (e.g., cluster headache), post-traumatic stress disorder (PTSD), anxiety, depression, neurodegenerative disorder, Alzheimer's disease, Parkinson's disease, psychological disorder, treatment resistant depression, suicidal ideation, major depressive disorder, bipolar disorder, schizophrenia, stroke, hypoxic brain injury, Chronic traumatic encephalopathy (CTE), traumatic brain injury, dementia, and addiction (e.g., substance use disorder). In some embodiments, the neurological disease is a migraine or cluster headache. In some embodiments, the neurological disease is a neurodegenerative disorder, dementia, Alzheimer's disease, or Parkinson's disease. In some embodiments, the neurological disease is a psychological disorder, treatment resistant depression, suicidal ideation, major depressive disorder, bipolar disorder, schizophrenia, post-traumatic stress disorder (PTSD), addiction (e.g., substance use disorder), depression, or anxiety. In some embodiments, the neuropsychiatric or neurological disease is a psychological disorder, treatment resistant depression, suicidal ideation, major depressive disorder, bipolar disorder, schizophrenia, post-traumatic stress disorder (PTSD), addiction (e.g., substance use disorder), depression, or anxiety. In some embodiments, the neuropsychiatric disease is a psychological disorder, treatment resistant depression, suicidal ideation, major depressive disorder, bipolar disorder, schizophrenia, post-traumatic stress disorder (PTSD), addiction (e.g., substance use disorder), depression, or anxiety. In some embodiments, the neuropsychiatric disease or neurological disease is addiction (e.g., substance use disorder). In some embodiments, the neuropsychiatric disease or neurological disease is depression. In some embodiments, the neuropsychiatric disease or neurological disease is anxiety.

In some embodiments, the neuropsychiatric disease or neurological disease is post-traumatic stress disorder (PTSD), addiction (e.g., substance use disorder), schizophrenia, depression, or anxiety. In some embodiments, the neuropsychiatric disease or neurological disease is addiction (e.g., substance use disorder). In some embodiments, the neuropsychiatric disease or neurological disease is depression. In some embodiments, the neuropsychiatric disease or neurological disease is anxiety. In some embodiments, the neuropsychiatric disease or neurological disease is post-traumatic stress disorder (PTSD). In some embodiments, the neurological disease is stroke or traumatic brain injury. In some embodiments, the neuropsychiatric disease or neurological disease is schizophrenia.

In some embodiments, the methods described herein are for increasing neuronal plasticity and has, for example, anti-addictive properties, antidepressant properties, anxiolytic properties, or a combination thereof. In some embodiments, decreased neuronal plasticity is associated with a neuropsychiatric disease. In some embodiments, a compound of the present disclosure is used for increasing neuronal plasticity. In some embodiments, the compounds described herein are used for treating a brain disorder. In some embodiments, the compounds described herein are used for increasing at least one of translation, transcription, or secretion of neurotrophic factors.

In some embodiments, the present disclosure provides a method of treating a disease, including administering to a subject in need thereof, a therapeutically effective amount of a compound of the present disclosure. In some embodiments, the disease is a musculoskeletal pain disorder including fibromyalgia, muscle pain, joint stiffness, osteoarthritis, rheumatoid arthritis, and muscle cramps. In some embodiments, the present invention provides a method of treating a disease of women's reproductive health including premenstrual dysphoric disorder (PMDD), premenstrual syndrome (PMS), post-partum depression, and menopause. In some embodiments, the disease or disorder is depression or a disease or disorder related to depression. In some embodiments, the disease or disorder is post-traumatic stress disorder. In some embodiments, the disease or disorder is fibromyalgia.

In yet another aspect, also provided herein are methods of treating fibromyalgia or a disease or disorder related to chronic widespread pain, fatigue or hypersensitivity, wherein the methods comprise administering to the subject a therapeutically effective amount of a 2C-B form described herein.

Diseases of particular interest that can be treated with the present compound forms include depression and related conditions. Accordingly, in some embodiments, the disease or disorder treated herein is depression or a disease or disorder related to depression. In some embodiments, the depression is major depressive disorder, persistent depressive disorder, bipolar disorder, treatment resistant depression (TRD), postpartum depression, premenstrual dysphoric disorder, or seasonal affective disorder. In some embodiments, the disease or disorder related to depression is anxiety. In some embodiments, methods of treating depression or a disease or disorder related to depression comprise treating the symptoms associated with the depression or the disease or disorder related to depression.

Described herein are methods of treating depression or a disease or disorder related to depression in a subject in need thereof, the method comprising administering to the subject a psychedelic 2C-B form and a serotonin receptor modulator, wherein the serotonin receptor modulator is administered at most about 3 hours prior to the release of the psychedelic. In some embodiments, the depression is major depressive disorder, persistent depressive disorder, bipolar disorder, treatment resistant depression (TRD), postpartum depression, premenstrual dysphoric disorder, or seasonal affective disorder. In some embodiments, the disease or disorder related to depression is anxiety. In some embodiments, methods of treating depression or a disease or disorder related to depression comprise treating the symptoms associated with the depression or the disease or disorder related to depression.

In some embodiments, the compounds of the present disclosure have activity as 5-HT_{2A} modulators. In some embodiments, the compounds of the present disclosure elicit a biological response by activating the 5-HT_{2A} receptor (e.g., allosteric modulation or modulation of a biological target that activates the 5-HT_{2A} receptor). 5-HT_{2A} agonism has been correlated with the promotion of neural plasticity (Ly et al., 2018). 5-HT_{2A} antagonists abrogate the neuritogenesis and spinogenesis effects of hallucinogenic compounds with 5-HT_{2A} agonist activity, for example, DMT, LSD, and DOI. In some embodiments, the compounds of the present disclosure are 5-HT_{2A} modulators and promote neural plasticity (e.g., cortical structural plasticity). In some embodiments, the compounds of the present disclosure are selective 5-HT_{2A} modulators and promote neural plasticity (e.g., cortical structural plasticity). In some embodiments, promotion of neural plasticity includes, for example, increased dendritic spine growth, increased synthesis of synaptic proteins, strengthened synaptic responses, increased dendritic arbor complexity, increased dendritic branch content, increased spinogenesis, increased neuritogenesis, or any combination thereof. In some embodiments, increased neural plasticity includes, for example, increased cortical structural plasticity in the anterior parts of the brain.

In some embodiments, the 5-HT_{2A} modulators (e.g., 5-HT_{2A} agonists) are non-hallucinogenic. In some embodiments, non-hallucinogenic 5-HT_{2A} modulators (e.g., 5-HT_{2A} agonists) are used to treat neurological diseases, which modulators do not elicit dissociative side-effects. In some embodiments, the hallucinogenic potential of the compounds described herein is assessed in vitro. In some embodiments, the hallucinogenic potential assessed in vitro of the compounds described herein is compared to the hallucinogenic potential assessed in vitro of hallucinogenic homologs. In some embodiments, the compounds described herein elicit less hallucinogenic potential in vitro than the hallucinogenic homologs. In some embodiments, the 2C-B forms function as 5-HT_{2A} modulators (e.g., 5-HT_{2A} agonists) that are non-hallucinogenic.

In some embodiments, serotonin receptor modulators, such as modulators of serotonin receptor 2A (5-HT_{2A} modulators, e.g., 5-HT_{2A} agonists), are used to treat a brain disorder. The presently disclosed compounds and compound forms can function as 5-HT_{2A} agonists alone, or in combination with a second therapeutic agent that also is a 5-HT_{2A} modulator. In such cases the second therapeutic agent can be an agonist or an antagonist. In some instances, it may be helpful to administer a 5-HT_{2A} antagonist in combination with a compound of the present disclosure to mitigate undesirable effects of 5-HT_{2A} agonism, such as potential hallucinogenic effects. Serotonin receptor modulators useful as second therapeutic agents for combination therapy as described herein are known to those of skill in the art and include, without limitation, ketanserin, volinanserin (MDL-100907), eplivanserin (SR-46349), pimavanserin (ACP-103), glemanserin (MDL-11939), ritanserin, flibanserin, nelotanserin, blonanserin, mianserin, mirtazapine, roluperiodone (CYR-101, MIN-101), quetiapine, olanzapine, altanserin, acepromazine, nefazodone, risperidone, pruvanserin, AC-90179, AC-279, adatanserin, fananserin, HY10275, benanserin, butanserin, manserin, iferanserin, lidanserin, pelanserin, seganserin, tropanserin, lorcaserin, ICI-169369, methiothepin, methysergide, trazodone, cinitapride, cyproheptadine, brexpiprazole, cariprazine, agomelatine, setoperone, 1-(1-Naphthyl)piperazine, LY-367265, pirenperone, metergoline, deramciclane, amperozide, cinanserin, LY-86057, GSK-215083, cyamemazine, mesulergine, BF-1, LY-215840, sergolexole, spiramide, LY-53857, amesergide, LY-108742, pipamperone, LY-314228, R91150, 5-MeO-NBpBrT, 9-Aminomethyl-9,10-dihydroanthracene, niaprazine, SB-215505, SB-204741, SB-206553, SB-242084, LY-272015, SB-243213, SB-200646, RS-102221, zotepine, clozapine, chlorpromazine, sertindole, iloperidone, paliperidone, asenapine, amisulpride, aripiprazole, lurasidone, ziprasidone, lumateperone, perospirone, mosapramine, AMDA (9-Aminomethyl-9,10-dihydroanthracene), methiothepin, an extended-release form of olanzapine (e.g., ZYPREXA RELPREVV), an extended-release form of quetiapine, an extended-release form of risperidone (e.g., Risperdal Consta), an extended-release form of paliperidone (e.g., Invega Sustenna and Invega Trinza), an extended-release form of fluphenazine decanoate including Prolixin Decanoate, an extended-release form of aripiprazole lauroxil including Aristada, and an extended-release form of aripiprazole including Abilify Maintena, or a pharmaceutically acceptable salt, solvate, metabolite, deuterated analog, derivative, prodrug, or combinations thereof.

In some embodiments, the serotonin receptor modulator for combination with the presently disclosed compound forms is selected from, glemanserin (MDL-11,939), eplivanserin (SR-46,349), ketanserin, ritanserin, altanserin, acepromazine, mianserin, mirtazapine, quetiapine, SB204741, SB206553, SB242084, LY272015, SB243213, blonanserin, SB200646, RS102221, nefazodone, volinanserin (MDL-100,907), pimavanserin (ACO-103), nelotanserin, lorcaserin, flibanserin, roluperiodone or a pharmaceutically acceptable salt, solvate, metabolite, deuterated analogue, derivative, prodrug, or combinations thereof. In some embodiments, the serotonin receptor modulator comprises eplivanserin (SR-46,349), ketanserin, ritanserin, altanserin, acepromazine, mianserin, mirtazapine, quetiapine, SB204741, SB206553, SB242084, LY272015, SB243213, blonanserin, SB200646, RS102221, nefazodone, MDL-100,907, pimavanserin, nelotanserin and lorcaserin.

In some embodiments, the serotonin receptor modulator used as a second therapeutic is pimavanserin or a pharmaceutically acceptable salt, solvate, metabolite, derivative, or prodrug thereof. In one embodiment, the serotonin receptor modulator is selected from the group consisting of eplivanserin, volinanserin, ketanserin, ritanserin, pimavanserin, nelotanserin, pruvanserin, flibanserin, olanzapine, quetiapine, and risperidone.

In some embodiments, the serotonin receptor modulator is ketanserin or a pharmaceutically acceptable salt, solvate, metabolite, deuterated analog, derivative, or prodrug thereof. In some embodiments, the serotonin receptor modulator is pimavanserin or a pharmaceutically acceptable salt, solvate, metabolite, deuterated analog, derivative, or prodrug thereof. In some embodiments, the serotonin receptor modulator is eplivanserin or a pharmaceutically acceptable salt, solvate, metabolite, deuterated analog, derivative, or prodrug thereof. In some embodiments, the serotonin receptor modulator is flibanserin or a pharmaceutically acceptable salt, solvate, metabolite, deuterated analog, derivative, or prodrug thereof. In some embodiments, the serotonin receptor modulator is roluperiodone or a pharmaceutically acceptable salt, solvate, metabolite, deuterated analog, derivative, or prodrug thereof.

In some embodiments, the serotonin receptor modulator is administered prior to a compound disclosed herein, such as about three or about one hour prior to administration of a compound disclosed herein. In some embodiments, the serotonin receptor modulator is administered at most about one hour prior to the presently disclosed compound. Thus, in some embodiments of combination therapy with the presently disclosed compounds, the second therapeutic agent is a serotonin receptor modulator. In some embodiments the second therapeutic agent serotonin receptor modulator is provided at a dose of from about 10 mg to about 350 mg. In some embodiments, the serotonin receptor modulator is provided at a dose of from about 20 mg to about 200 mg. In some embodiments, the serotonin receptor modulator is provided at a dose of from about 10 mg to about 100 mg. In certain such embodiments, the compound of the present disclosure is provided at a dose of from about 10 mg to about 100 mg, or from about 20 to about 200 mg, or from about 15 to about 300 mg, and the serotonin receptor modulator is provided at a dose of about 10 mg to about 100 mg.

In some embodiments, the serotonin receptor modulator is administered prior to a compound disclosed herein, such as about three or about one hours prior to administration of a compound disclosed herein. In some embodiments, the serotonin receptor modulator is administered at most about one hour prior to the presently disclosed compound. Thus, in some embodiments of combination therapy with the presently disclosed compounds, the second therapeutic agent is a serotonin receptor modulator.

In some embodiments, non-hallucinogenic 5-HT_{2A} modulators (e.g., 5-HT_{2A} agonists) are used to treat neurological diseases. In some embodiments, the neurological diseases comprise decreased neural plasticity, decreased cortical structural plasticity, decreased 5-HT_{2A} receptor content, decreased dendritic arbor complexity, loss of dendritic spines, decreased dendritic branch content, decreased spinogenesis, decreased neuritogenesis, retraction of neurites, or any combination thereof.

In some embodiments, non-hallucinogenic 5-HT_{2A} modulators (e.g., 5-HT_{2A} agonists) are used for increasing neuronal plasticity. In some embodiments, non-hallucinogenic 5-HT_{2A} modulators (e.g., 5-HT_{2A} agonists) are used for treating a brain disorder. In some embodiments, non-hallucinogenic 5-HT_{2A} modulators (e.g., 5-HT_{2A} agonists) are used for increasing at least one of translation, transcription, or secretion of neurotrophic factors.

In some embodiments the presently disclosed compounds are given to patients in a low dose that is lower than would produce noticeable psychedelic effects but high enough to provide a therapeutic benefit. This dose range is predicted to be between 200 ug (micrograms) and 2 mg.

### Methods for Increasing Neuronal Plasticity

Neuronal plasticity refers to the ability of the brain to change structure and/or function throughout a subject's life. New neurons can be produced and integrated into the central nervous system throughout the subject's life. Increasing neuronal plasticity includes, but is not limited to, promoting neuronal growth, promoting neuritogenesis, promoting synaptogenesis, promoting dendritogenesis, increasing dendritic arbor complexity, increasing dendritic spine density, and increasing excitatory synapsis in the brain. In some embodiments, increasing neuronal plasticity comprises promoting neuronal growth, promoting neuritogenesis, promoting synaptogenesis, promoting dendritogenesis, increasing dendritic arbor complexity, and increasing dendritic spine density.

In some embodiments, increasing neuronal plasticity by treating a subject with a disclosed compound can treat neurodegenerative disorder, Alzheimer's, Parkinson's disease, psychological disorder, depression, addiction, anxiety, post-traumatic stress disorder, treatment resistant depression, suicidal ideation, major depressive disorder, bipolar disorder, schizophrenia, stroke, traumatic brain injury, or substance use disorder.

In some embodiments, the present disclosure provides methods for increasing neuronal plasticity, comprising contacting a neuronal cell with a compound of the present disclosure. In some embodiments, increasing neuronal plasticity improves a brain disorder described herein.

In some embodiments, a compound of the present disclosure is used to increase neuronal plasticity. In some embodiments, the compounds used to increase neuronal plasticity have, for example, anti-addictive properties, antidepressant properties, anxiolytic properties, or a combination thereof. In some embodiments, decreased neuronal plasticity is associated with a neuropsychiatric disease. In some embodiments, the neuropsychiatric disease is a mood or anxiety disorder. In some embodiments, the neuropsychiatric disease includes, for example, migraine, cluster headache, post-traumatic stress disorder (PTSD), schizophrenia, anxiety, depression, and addiction (e.g., substance abuse disorder). In some embodiments, brain disorders include, for example, migraines, addiction (e.g., substance use disorder), depression, and anxiety.

In some embodiments, the experiment or assay to determine increased neuronal plasticity of any compound of the present disclosure is a phenotypic assay, a dendritogenesis assay, a spinogenesis assay, a synaptogenesis assay, a Sholl analysis, a concentration-response experiment, a 5-HT_{2A} agonist assay, a 5-HT_{2A} antagonist assay, a 5-HT_{2A} binding assay, or a 5-HT_{2A} blocking experiment (e.g., ketanserin blocking experiments). In some embodiments, the experiment or assay to determine the hallucinogenic potential of any compound of the present invention is a mouse head-twitch response (HTR) assay.

In some embodiments, the present disclosure provides a method for increasing neuronal plasticity, comprising contacting a neuronal cell with a compound disclosed herein.

### Methods of Treating a Brain Disorder

In some embodiments, the present disclosure provides a method of treating a disease, including administering to a subject in need thereof, a therapeutically effective amount of a compound of the present disclosure. In some embodiments, the disease is a musculoskeletal pain disorder including fibromyalgia, muscle pain, joint stiffness, osteoarthritis, rheumatoid arthritis, muscle cramps. In some embodiments, the present disclosure provides a method of treating a disease of women's reproductive health including premenstrual dysphoric disorder (PMDD), premenstrual syndrome (PMS), post-partum depression, and menopause. In some embodiments, the present disclosure provides a method of treating a brain disorder, including administering to a subject in need thereof, a therapeutically effective amount of a compound of the present disclosure. In some embodiments, the present disclosure provides a method of treating a brain disorder with combination therapy, including administering to a subject in need thereof, a therapeutically effective amount of a compound of the present disclosure and at least one additional therapeutic agent.

In some embodiments, 5-HT_{2A} modulators (e.g., 5-HT_{2A} agonists) are used to treat a brain disorder. In some embodiments, the brain disorders comprise decreased neural plasticity, decreased cortical structural plasticity, decreased 5-HT_{2A} receptor content, decreased dendritic arbor complexity, loss of dendritic spines, decreased dendritic branch content, decreased spinogenesis, decreased neuritogenesis, retraction of neurites, or any combination thereof.

In some embodiments, a compound of the present disclosure is used to treat brain disorders. In some embodiments, the compounds have, for example, anti-addictive properties, antidepressant properties, anxiolytic properties, or a combination thereof. In some embodiments, the brain disorder is a neuropsychiatric disease. In some embodiments, the neuropsychiatric disease is a mood or anxiety disorder. In some embodiments, brain disorders include, for example, migraine, cluster headache, post-traumatic stress disorder (PTSD), anxiety, depression, panic disorder, suicidality, schizophrenia, and addiction (e.g., substance abuse disorder). In some embodiments, brain disorders include, for example, migraines, addiction (e.g., substance use disorder), depression, and anxiety.

In some embodiments, the present disclosure provides a method of treating a brain disorder, comprising administering to a subject in need thereof a therapeutically effective amount of a compound disclosed herein.

In some embodiments, the brain disorder is a neurodegenerative disorder, Alzheimer's, Parkinson's disease, psychological disorder, depression, addiction, anxiety, post-traumatic stress disorder, treatment resistant depression, suicidal ideation, major depressive disorder, bipolar disorder, schizophrenia, stroke, traumatic brain injury, or substance use disorder.

In some embodiments, the brain disorder is a neurodegenerative disorder, Alzheimer's, or Parkinson's disease. In some embodiments, the brain disorder is a psychological disorder, depression, addiction, anxiety, or a post-traumatic stress disorder. In some embodiments, the brain disorder is depression. In some embodiments, the brain disorder is addiction. In some embodiments, the brain disorder is treatment resistant depression, suicidal ideation, major depressive disorder, bipolar disorder, schizophrenia, stroke, traumatic brain injury or substance use disorder. In some embodiments, the brain disorder is treatment resistant depression, suicidal ideation, major depressive disorder, bipolar disorder, schizophrenia, or substance use disorder. In some embodiments, the brain disorder is stroke or traumatic brain injury. In some embodiments, the brain disorder is treatment resistant depression, suicidal ideation, major depressive disorder, bipolar disorder, or substance use disorder. In some embodiments, the brain disorder is schizophrenia. In some embodiments, the brain disorder is alcohol use disorder.

In some embodiments, the method further comprises administering one or more additional therapeutic agent that is lithium, olanzapine (Zyprexa), quetiapine (Seroquel), risperidone (Risperdal), ariprazole (Abilify), ziprasidone (Geodon), clozapine (Clozaril), divalproex sodium (Depakote), lamotrigine (Lamictal), valproic acid (Depakene), carbamazepine (Equetro), topiramate (Topamax), levomilnacipran (Fetzima), duloxetine (Cymbalta, Yentreve), venlafaxine (Effexor), citalopram (Celexa), fluvoxamine (Luvox), escitalopram (Lexapro), fluoxetine (Prozac), paroxetine (Paxil), sertraline (Zoloft), clomipramine (Anafranil), amitriptyline (Elavil), desipramine (Norpramin), imipramine (Tofranil), nortriptyline (Pamelor), phenelzine (Nardil), tranylcypromine (Parnate), diazepam (Valium), alprazolam (Xanax), or clonazepam (Klonopin).

In certain embodiments of the method for treating a brain disorder with a solid form disclosed herein, a second therapeutic agent that is an empathogenic agent is administered. Examples of suitable empathogenic agents for use in combination with the present solid forms include phenethylamines, such as 3,4-methylenedioxymethamphetamine (MDMA), a solid form of MDMA disclosed herein, and analogs thereof. Other suitable empathogenic agents for use in combination with the presently disclosed salts and solid forms include, without limitation,
N-Allyl-3,4-methylenedioxy-amphetamine (MDAL)
N-Butyl-3,4-methylenedioxyamphetamine (MDBU)
N-Benzyl-3,4-methylenedioxyamphetamine (MDBZ)
N-Cyclopropylmethyl-3,4-methylenedioxyamphetamine (MDCPM)
N,N-Dimethyl-3,4-methylenedioxyamphetamine (MDDM)
N-Ethyl-3,4-methylenedioxyamphetamine (MDE; MDEA)
N-(2-Hydroxyethyl)-3,4-methylenedioxy amphetamine (MDHOET)
N-Isopropyl-3,4-methylenedioxyamphetamine (MDIP)
N-Methyl-3,4-ethylenedioxyamphetamine (MDMC)
N-Methoxy-3,4-methylenedioxyamphetamine (MDMEO)
N-(2-Methoxyethyl)-3,4-methylenedioxyamphetamine (MDMEOET)
alpha,alpha,N-Trimethyl-3,4-methylenedioxyphenethylamine (MDMP;
3,4-Methylenedioxy-N-methylphentermine)
N-Hydroxy-3,4-methylenedioxyamphetamine (MDOH)
3,4-Methylenedioxyphenethylamine (MDPEA)
alpha,alpha-Dimethyl-3,4-methylenedioxyphenethylamine (MDPH; 3,4-methylenedioxyphentermine)
N-Propargyl-3,4-methylenedioxyamphetamine (MDPL)
Methylenedioxy-2-aminoindane (MDAI)
1,3-Benzodioxolyl-N-methylbutanamine (MBDB)
3,4-methylenedioxy-N-methyl-α-ethylphenylethylamine
3,4-Methylenedioxyamphetamine (MDA)
Methylone (also known as “3,4-methylenedioxy-N-methylcathinone)
Ethylone (also known as 3,4-methylenedioxy-N-ethylcathinone)
GHB (also known as gamma hydroxybutyrate or sodium oxybate)
N-Propyl-3,4-methylenedioxyamphetamine (MDPR), and the like.

In some embodiments, the compounds of the present disclosure are used in combination with the standard of care therapy for a neurological disease described herein. Non-limiting examples of the standard of care therapies, may include, for example, lithium, olanzapine, quetiapine, risperidone, ariprazole, ziprasidone, clozapine, divalproex sodium, lamotrigine, valproic acid, carbamazepine, topiramate, levomilnacipran, duloxetine, venlafaxine, citalopram, fluvoxamine, escitalopram, fluoxetine, paroxetine, sertraline, clomipramine, amitriptyline, desipramine, imipramine, nortriptyline, phenelzine, tranylcypromine, diazepam, alprazolam, clonazepam, or any combination thereof. Nonlimiting examples of standard of care therapy for depression are sertraline, fluoxetine, escitalopram, venlafaxine, or aripiprazole. Non-limiting examples of standard of care therapy for depression are citralopram, escitalopram, fluoxetine, paroxetine, diazepam, or sertraline. Additional examples of standard of care therapeutics are known to those of ordinary skill in the art.

### Methods of Increasing at Least One of Translation, Transcription, or Secretion of Neurotrophic Factors

Neurotrophic factors refers to a family of soluble peptides or proteins which support the survival, growth, and differentiation of developing and mature neurons. Increasing at least one of translation, transcription, or secretion of neurotrophic factors can be useful for, but not limited to, increasing neuronal plasticity, promoting neuronal growth, promoting neuritogenesis, promoting synaptogenesis, promoting dendritogenesis, increasing dendritic arbor complexity, increasing dendritic spine density, and increasing excitatory synapsis in the brain. In some embodiments, increasing at least one of translation, transcription, or secretion of neurotrophic factors can increasing neuronal plasticity. In some embodiments, increasing at least one of translation, transcription, or secretion of neurotrophic factors can promoting neuronal growth, promoting neuritogenesis, promoting synaptogenesis, promoting dendritogenesis, increasing dendritic arbor complexity, and/or increasing dendritic spine density.

In some embodiments, 5-HT_{2A} modulators (e.g., 5-HT_{2A} agonists) are used to increase at least one of translation, transcription, or secretion of neurotrophic factors. In some embodiments, a compound of the present disclosure is used to increase at least one of translation, transcription, or secretion of neurotrophic factors. In some embodiments, increasing at least one of translation, transcription or secretion of neurotrophic factors treats a migraine, headaches (e.g., cluster headache), post-traumatic stress disorder (PTSD), anxiety, depression, neurodegenerative disorder, Alzheimer's disease, Parkinson's disease, psychological disorder, treatment resistant depression, suicidal ideation, major depressive disorder, bipolar disorder, schizophrenia, stroke, traumatic brain injury, and addiction (e.g., substance use disorder).

In some embodiments, the experiment or assay used to determine increase translation of neurotrophic factors includes ELISA, western blot, immunofluorescence assays, proteomic experiments, and mass spectrometry. In some embodiments, the experiment or assay used to determine increase transcription of neurotrophic factors includes gene expression assays, PCR, and microarrays. In some embodiments, the experiment or assay used to determine increase secretion of neurotrophic factors includes ELISA, western blot, immunofluorescence assays, proteomic experiments, and mass spectrometry.

In some embodiments, the present disclosure provides a method for increasing at least one of translation, transcription or secretion of neurotrophic factors, comprising contacting a neuronal cell with a compound disclosed herein.

Combination Therapy

In particular embodiments of treating the disorders described above, combination therapy is used as described herein. In such therapy a form of 2C-B or 2C-B.HCl described herein is administered in combination with a serotonin receptor modulator. In certain embodiments the serotonin receptor modulator is selected from the group consisting of altanserin, blonanserin, eplivanserin, glemanserin, volinanserin, ketanserin, ritanserin, pimavanserin, nelotanserin, pruvanserin, and flibanserin. In one embodiment, the serotonin receptor modulator is selected from the group consisting of serotonin receptor modulator is selected from the group consisting of eplivanserin, volinanserin, ketanserin, ritanserin, pimavanserin, nelotanserin, pruvanserin, and flibanserin.

In some embodiments, the serotonin receptor modulator for use with the psychedelic 2C-B.HCl is eplivanserin and, wherein the eplivanserin is administered in about 1 mg to about 40 mg, or about 5 mg to about 10 mg, and the 2C-B HCl is administered between about 1 mg and 40 mg. In some embodiments, the amount of 2C-B is provided on a 2C-B basis.

In some embodiments, the serotonin receptor modulator is volinanserin and the psychedelic is 2C-B.HCl, wherein the volinanserin is administered in about 1 mg to about 60 mg, or about 5 mg to about 20 mg, and the 2C-B.HCl is administered between about 1 mg and 40 mg. In some embodiments, the amount of 2C-B is provided on a 2C-B basis.

In some embodiments, the serotonin receptor modulator for use with the psychedelic 2C-B.HCl is ketanserin, wherein the ketanserin is administered in about 10 mg to about 80 mg, about 30 mg to about 50 mg, or about 40 mg and the 2C-B.HCl is administered between about 1 mg and 40 mg. In some embodiments, the amount of 2C-B is provided on a 2C-B basis.

In some embodiments, the serotonin receptor modulator for use with the psychedelic 2C-B.HCl is ritanserin, wherein the ritanserin is administered in about 1 mg to about 40 mg, or about 2.5 mg to about 10 mg, and the 2C-B.HCl is administered between about 1 mg and 40 mg. In some embodiments, the amount of 2C-B is provided on a 2C-B basis.

In some embodiments, the serotonin receptor modulator is pimavanserin and the psychedelic is 2C-B.HCl, wherein the pimavanserin is administered in about 1 mg to about 60 mg, or about 17 mg to about 34 mg, and the 2C-B HCl is administered between about 1 mg and 40 mg. In some embodiments, the amount of 2C-B is provided on a 2C-B basis.

In some embodiments, the serotonin receptor modulator for use with the psychedelic 2C-B.HCl is nelotanserin, wherein the nelotanserin is administered in about 1 mg to about 80 mg, or about 40 mg to about 80 mg, and the 2C-B HCl is administered between about 1 mg and 40 mg. In some embodiments, the amount of 2C-B is provided on a 2C-B basis.

In some embodiments, the serotonin receptor modulator for use with the psychedelic 2C-B.HCl is pruvanserin, wherein the pruvanserin is administered in about 1 mg to about 40 mg, or about 3 mg to about 10 mg, and the 2C-B.HCl is administered between about 1 mg and 40 mg. In some embodiments, the amount of 2C-B is provided on a 2C-B basis.

In some embodiments, the serotonin receptor modulator for use with the psychedelic 2C-B.HCl is flibanserin, wherein the flibanserin is administered in about 10 mg to about 200 mg, or about 80 mg to about 120 mg, or about 100 mg and the 2C-B.HCl is administered between about 1 mg and 40 mg. In some embodiments, the amount of 2C-B is provided on a 2C-B basis.

In some embodiments, the serotonin receptor modulator for use with the psychedelic 2C-B is eplivanserin and, wherein the eplivanserin is administered in about 1 mg to about 40 mg, or about 5 mg to about 10 mg, and the 2C-B form is administered between about 1 mg and 40 mg. In some embodiments, the amount of the 2C-B form is provided on a 2C-B basis.

In some embodiments, the serotonin receptor modulator is volinanserin and the psychedelic is 2C-B, wherein the volinanserin is administered in about 1 mg to about 60 mg, or about 5 mg to about 20 mg, and the 2C-B form is administered between about 1 mg and 40 mg. In some embodiments, the amount of the 2C-B form is provided on a 2C-B basis.

In some embodiments, the serotonin receptor modulator for use with the psychedelic 2C-B is ketanserin, wherein the ketanserin is administered in about 10 mg to about 80 mg, about 30 mg to about 50 mg, or about 40 mg and the 2C-B form is administered between about 1 mg and 40 mg. In some embodiments, the amount of the 2C-B form is provided on a 2C-B basis.

In some embodiments, the serotonin receptor modulator for use with the psychedelic 2C-B is ritanserin, wherein the ritanserin is administered in about 1 mg to about 40 mg, or about 2.5 mg to about 10 mg, and the 2C-B form is administered between about 1 mg and 40 mg. In some embodiments, the amount of the 2C-B form is provided on a 2C-B basis.

In some embodiments, the serotonin receptor modulator is pimavanserin and the psychedelic is 2C-B, wherein the pimavanserin is administered in about 1 mg to about 60 mg, or about 17 mg to about 34 mg, and the 2C-B form is administered between about 1 mg and 40 mg. In some embodiments, the amount of the 2C-B form is provided on a 2C-B basis.

In some embodiments, the serotonin receptor modulator for use with the psychedelic 2C-B is nelotanserin, wherein the nelotanserin is administered in about 1 mg to about 80 mg, or about 40 mg to about 80 mg, and the 2C-B form is administered between about 1 mg and 40 mg. In some embodiments, the amount of the 2C-B form is provided on a 2C-B basis.

In some embodiments, the serotonin receptor modulator for use with the psychedelic 2C-B is pruvanserin, wherein the pruvanserin is administered in about 1 mg to about 40 mg, or about 3 mg to about 10 mg, and the 2C-B form is administered between about 1 mg and 40 mg. In some embodiments, the amount of the 2C-B form is provided on a 2C-B basis.

In some embodiments, the serotonin receptor modulator for use with the psychedelic 2C-B is flibanserin, wherein the flibanserin is administered in about 10 mg to about 200 mg, or about 80 mg to about 120 mg, or about 100 mg and the 2C-B form is administered between about 1 mg and 40 mg. In some embodiments, the amount of the 2C-B form is provided on a 2C-B basis.

In some embodiments, the serotonin receptor modulator for use with the 2C-B HCl forms disclosed herein, including those described in Table 3, is eplivanserin, wherein the eplivanserin is administered in about 1 mg to about 40 mg, or about 5 mg to about 10 mg, and the 2C-B HCl form disclosed herein, including those described in Table 3, is administered between about 1 mg and 40 mg.

In some embodiments, the serotonin receptor modulator for use with the 2C-B HCl forms disclosed herein, including those described in Table 3, is volinanserin, wherein the volinanserin is administered in about 1 mg to about 60 mg, or about 5 mg to about 20 mg, and the 2C-B HCl form disclosed herein, including those described in Table 3, is administered between about 1 mg and 40 mg.

In some embodiments, the serotonin receptor modulator for use with the 2C-B HCl forms disclosed herein, including those described in Table 3, is ketanserin, wherein the ketanserin is administered in about 10 mg to about 80 mg, about 30 mg to about 50 mg, or about 40 mg and the 2C-B HCl form disclosed herein, including those described in Table 3, is administered between about 1 mg and 40 mg.

In some embodiments, the serotonin receptor modulator for use with the 2C-B HCl forms disclosed herein, including those described in Table 3, is ritanserin, wherein the ritanserin is administered in about 1 mg to about 40 mg, or about 2.5 mg to about 10 mg, and the 2C-B HCl form disclosed herein, including those described in Table 3, is administered between about 1 mg and 40 mg.

In some embodiments, the serotonin receptor modulator is pimavanserin and the 2C-B HCl form disclosed herein, including those described in Table 3, wherein the pimavanserin is administered in about 1 mg to about 60 mg, or about 17 mg to about 34 mg, and the 2C-B HCl form disclosed herein, including those described in Table 3, is administered between about 1 mg and 40 mg.

In some embodiments, the serotonin receptor modulator for use with the 2C-B HCl form disclosed herein, including those described in Table 3, is nelotanserin, wherein the nelotanserin is administered in about 1 mg to about 80 mg, or about 40 mg to about 80 mg, and the 2C-B HCl form disclosed herein, including those described in Table 3, is administered between about 1 mg and 40 mg.

In some embodiments, the serotonin receptor modulator for use with the 2C-B HCl form disclosed herein, including those described in Table 3, is pruvanserin, wherein the pruvanserin is administered in about 1 mg to about 40 mg, or about 3 mg to about 10 mg, and the 2C-B HCl form disclosed herein, including those described in Table 3, is administered between about 1 mg and 40 mg.

In some embodiments, the serotonin receptor modulator for use with the 2C-B HCl form disclosed herein, including those described in Table 3, is flibanserin, wherein the flibanserin is administered in about 10 mg to about 200 mg, or about 80 mg to about 120 mg, or about 100 mg and the 2C-B HCl form disclosed herein, including those described in Table 3, is administered between about 1 mg and 40 mg.

In some embodiments, the serotonin receptor modulator for use with the 2C-B HCl form disclosed herein, including those described in Table 3, is olanzapine, wherein the olanzapine is administered in about 2.5 mg to about 30 mg, or about 5 mg or about 10 mg, or about 20 mg or about 25 mg, and the 2C-B HCl form disclosed herein, including those described in Table 3, is administered between about 1 mg and 40 mg.

In some embodiments, the serotonin receptor modulator for use with the 2C-B HCl form disclosed herein, including those described in Table 3, is an extended-release of olanzapine such as ZYPREXA RELPREVV, wherein the extended release olanzapine is administered in about 50 mg to about 450 mg, or about 150 mg or about 210 mg, or about 300 mg or about 405 mg, and the 2C-B HCl form disclosed herein, including those described in Table 3, is administered between about 1 mg and 40 mg.

In some embodiments, the serotonin receptor modulator for use with the 2C-B HCl form disclosed herein, including those described in Table 3, is quetiapine, wherein the quetiapine is administered in about 25 mg to about 800 mg, or about 50 mg to about 100 mg, or about 150 mg or about 200 mg or about 250 mg or about 300 mg, and the 2C-B HCl form disclosed herein, including those described in Table 3, is administered between about 1 mg and 40 mg.

In some embodiments, the serotonin receptor modulator for use with the 2C-B HCl form disclosed herein, including those described in Table 3, is an extended-release of quetiapine, wherein the extended-release of quetiapine is administered in about 50 mg to about 300 mg, or about 50 mg or about 100 mg or about 200 mg, or about 300 mg, and the 2C-B HCl form disclosed herein, including those described in Table 3, is administered between about 1 mg and 40 mg.

In some embodiments, the serotonin receptor modulator for use with the 2C-B HCl form disclosed herein, including those described in Table 3, is risperidone, wherein the risperidone is administered in about 0.5 mg to about 20 mg or about 5 mg, or about 1 mg, or about 2 mg, or about 3 mg or about 4 mg or about 5 mg or about 7.5 mg or about 10 mg or about 16 mg, and the 2C-B HCl form disclosed herein, including those described in Table 3, is administered between about 1 mg and 40 mg.

In some embodiments, the serotonin receptor modulator for use with the 2C-B HCl form disclosed herein, including those described in Table 3, is an extended-release of risperidone including (RISPERDAL CONSTA), wherein the extended-release of risperidone is administered in about 12.5 mg, or about 25 mg, or about 37.5 mg, or about 50 mg, and the 2C-B HCl form disclosed herein, including those described in Table 3, is administered between about 1 mg and 40 mg.

In some embodiments, the serotonin receptor modulator for use with the 2C-B salt and solid forms disclosed herein, including those described in Table 18, is eplivanserin and, wherein the eplivanserin is administered in about 1 mg to about 40 mg, or about 5 mg to about 10 mg, and the 2C-B form disclosed herein, including those described in Table 18, is administered between about 1 mg and 40 mg. In some embodiments, the amount of the 2C-B form is provided on a 2C-B basis.

In some embodiments, the serotonin receptor modulator for use with the 2C-B salt and solid forms disclosed herein, including those described in Table 18, is volinanserin, wherein the volinanserin is administered in about 1 mg to about 60 mg, or about 5 mg to about 20 mg, and the 2C-B form disclosed herein, including those described in Table 18, is administered between about 1 mg and 40 mg. In some embodiments, the amount of the 2C-B form is provided on a 2C-B basis.

In some embodiments, the serotonin receptor modulator for use with the 2C-B salt and solid forms disclosed herein, including those described in Table 18, is ketanserin, wherein the ketanserin is administered in about 10 mg to about 80 mg, about 30 mg to about 50 mg, or about 40 mg and the 2C-B form disclosed herein, including those described in Table 18, is administered between about 1 mg and 40 mg. In some embodiments, the amount of the 2C-B form is provided on a 2C-B basis.

In some embodiments, the serotonin receptor modulator for use with the 2C-B salt and solid forms disclosed herein, including those described in Table 18, is ritanserin, wherein the ritanserin is administered in about 1 mg to about 40 mg, or about 2.5 mg to about 10 mg, and the 2C-B form disclosed herein, including those described in Table 18, is administered between about 1 mg and 40 mg. In some embodiments, the amount of the 2C-B form is provided on a 2C-B basis.

In some embodiments, the serotonin receptor modulator for use with the 2C-B salt and solid forms disclosed herein, including those described in Table 18, is pimavanserin, wherein the pimavanserin is administered in about 1 mg to about 60 mg, or about 17 mg to about 34 mg, and the 2C-B form disclosed herein, including those described in Table 18, is administered between about 1 mg and 40 mg. In some embodiments, the amount of the 2C-B form is provided on a 2C-B basis.

In some embodiments, the serotonin receptor modulator for use with the 2C-B salt and solid forms disclosed herein, including those described in Table 18, is nelotanserin, wherein the nelotanserin is administered in about 1 mg to about 80 mg, or about 40 mg to about 80 mg, and the 2C-B form disclosed herein, including those described in Table 18, is administered between about 1 mg and 40 mg. In some embodiments, the amount of the 2C-B form is provided on a 2C-B basis.

In some embodiments, the serotonin receptor modulator for use with the 2C-B salt and solid forms disclosed herein, including those described in Table 18, is pruvanserin, wherein the pruvanserin is administered in about 1 mg to about 40 mg, or about 3 mg to about 10 mg, and the 2C-B form disclosed herein, including those described in Table 18, is administered between about 1 mg and 40 mg. In some embodiments, the amount of the 2C-B form is provided on a 2C-B basis.

In some embodiments, the serotonin receptor modulator for use with the 2C-B salt and solid forms disclosed herein, including those described in Table 18, is flibanserin, wherein the flibanserin is administered in about 10 mg to about 200 mg, or about 80 mg to about 120 mg, or about 100 mg, and the 2C-B form disclosed herein, including those described in Table 18, is administered between about 1 mg and 40 mg. In some embodiments, the amount of the 2C-B form is provided on a 2C-B basis.

In some embodiments, the serotonin receptor modulator for use with the 2C-B salt and solid forms disclosed herein, including those described in Table 18, is olanzapine, wherein the olanzapine is administered in about 2.5 mg to about 30 mg, or about 5 mg or about 10 mg, or about 20 mg or about 25 mg, and the 2C-B form disclosed herein, including those described in Table 18, is administered between about 1 mg and 40 mg. In some embodiments, the amount of the 2C-B form is provided on a 2C-B basis.

In some embodiments, the serotonin receptor modulator for use with the 2C-B salt and solid forms disclosed herein, including those described in Table 18, is an extended-release of olanzapine such as ZYPREXA RELPREVV, wherein the extended release olanzapine is administered in about 50 mg to about 450 mg, or about 150 mg or about 210 mg, or about 300 mg or about 405 mg, and the 2C-B form disclosed herein, including those described in Table 18, is administered between about 1 mg and 40 mg. In some embodiments, the amount of the 2C-B form is provided on a 2C-B basis.

In some embodiments, the serotonin receptor modulator for use with the 2C-B salt and solid forms disclosed herein, including those described in Table 18, is quetiapine, wherein the quetiapine is administered in about 25 mg to about 800 mg, or about 50 mg to about 100 mg, or about 150 mg or about 200 mg or about 250 mg or about 300 mg, and the 2C-B form disclosed herein, including those described in Table 18, is administered between about 1 mg and 40 mg. In some embodiments, the amount of the 2C-B form is provided on a 2C-B basis.

In some embodiments, the serotonin receptor modulator for use with the 2C-B salt and solid forms disclosed herein, including those described in Table 18, is an extended-release of quetiapine, wherein the extended-release of quetiapine is administered in about 50 mg to about 300 mg, or about 50 mg or about 100 mg or about 200 mg, or about 300 mg, and the 2C-B form disclosed herein, including those described in Table 18, is administered between about 1 mg and 40 mg. In some embodiments, the amount of the 2C-B form is provided on a 2C-B basis.

In some embodiments, the serotonin receptor modulator for use with the 2C-B salt and solid forms disclosed herein, including those described in Table 18, is risperidone, wherein the risperidone is administered in about 0.5 mg to about 20 mg or about 0.5 mg, or about 1 mg, or about 2 mg, or about 3 mg or about 4 mg or about 5 mg or about 7.5 mg or about 10 mg or about 16 mg, and the 2C-B form disclosed herein, including those described in Table 18, is administered between about 1 mg and 40 mg. In some embodiments, the amount of the 2C-B form is provided on a 2C-B basis.

In some embodiments, the serotonin receptor modulator for use with the 2C-B salt and solid forms disclosed herein, including those described in Table 18, is an extended-release of risperidone including (RISPERDAL CONSTA), wherein the extended-release of risperidone is administered in about 12.5 mg, or about 25 mg, or about 37.5 mg, or about 50 mg, and the 2C-B form disclosed herein, including those described in Table 18, is administered between about 1 mg and 40 mg. In some embodiments, the amount of the 2C-B form is provided on a 2C-B basis.

In some embodiments, the serotonin receptor modulator for use with the 2C-B tartrate Form 1 is eplivanserin, wherein the eplivanserin is administered in about 1 mg to about 40 mg, or about 5 mg to about 10 mg, and the 2C-B tartrate Form 1 is administered between about 1 mg and 40 mg.

In some embodiments, the serotonin receptor modulator for use with the 2C-B tartrate Form 1 is volinanserin, wherein the volinanserin is administered in about 1 mg to about 60 mg, or about 5 mg to about 20 mg, and the 2C-B tartrate Form 1 is administered between about 1 mg and 40 mg.

In some embodiments, the serotonin receptor modulator for use with the 2C-B tartrate Form 1 is ketanserin, wherein the ketanserin is administered in about 10 mg to about 80 mg, about 30 mg to about 50 mg, or about 40 mg and the 2C-B tartrate Form 1 is administered between about 1 mg and 40 mg.

In some embodiments, the serotonin receptor modulator for use with the 2C-B tartrate Form 1 is ritanserin, wherein the ritanserin is administered in about 1 mg to about 40 mg, or about 2.5 mg to about 10 mg, and the 2C-B tartrate Form 1 is administered between about 1 mg and 40 mg.

In some embodiments, the serotonin receptor modulator is pimavanserin and the 2C-B tartrate Form 1 wherein the pimavanserin is administered in about 1 mg to about 60 mg, or about 17 mg to about 34 mg, and the 2C-B tartrate Form 1 is administered between about 1 mg and 40 mg.

In some embodiments, the serotonin receptor modulator for use with the 2C-B tartrate Form 1 is nelotanserin, wherein the nelotanserin is administered in about 1 mg to about 80 mg, or about 40 mg to about 80 mg, and the 2C-B tartrate Form 1 is administered between about 1 mg and 40 mg.

In some embodiments, the serotonin receptor modulator for use with the 2C-B tartrate Form 1 is pruvanserin, wherein the pruvanserin is administered in about 1 mg to about 40 mg, or about 3 mg to about 10 mg, and the 2C-B tartrate Form 1 is administered between about 1 mg and 40 mg.

In some embodiments, the serotonin receptor modulator for use with the 2C-B tartrate Form 1 is flibanserin, wherein the flibanserin is administered in about 10 mg to about 200 mg, or about 80 mg to about 120 mg, or about 100 mg and the 2C-B tartrate Form 1 is administered between about 1 mg and 40 mg.

In some embodiments, the serotonin receptor modulator for use with the 2C-B tartrate Form 1 is olanzapine, wherein the olanzapine is administered in about 2.5 mg to about 30 mg, or about 5 mg or about 10 mg, or about 20 mg or about 25 mg, and the 2C-B tartrate Form 1 is administered between about 1 mg and 40 mg.

In some embodiments, the serotonin receptor modulator for use with the 2C-B tartrate Form 1 is an extended-release of olanzapine such as ZYPREXA RELPREVV, wherein the extended release olanzapine is administered in about 50 mg to about 450 mg, or about 150 mg or about 210 mg, or about 300 mg or about 405 mg, and the 2C-B tartrate Form 1 is administered between about 1 mg and 40 mg.

In some embodiments, the serotonin receptor modulator for use with the 2C-B tartrate Form 1 is quetiapine, wherein the quetiapine is administered in about 25 mg to about 800 mg, or about 50 mg to about 100 mg, or about 150 mg or about 200 mg or about 250 mg or about 300 mg, and the 2C-B tartrate Form 1 is administered between about 1 mg and 40 mg.

In some embodiments, the serotonin receptor modulator for use with the 2C-B tartrate Form 1 is an extended-release of quetiapine, wherein the extended-release of quetiapine is administered in about 50 mg to about 300 mg, or about 50 mg or about 100 mg or about 200 mg, or about 300 mg, and the 2C-B tartrate Form 1 is administered between about 1 mg and 40 mg.

In some embodiments, the serotonin receptor modulator for use with the 2C-B tartrate Form 1 is risperidone, wherein the risperidone is administered in about 0.5 mg to about 20 mg or about 5 mg, or about 1 mg, or about 2 mg, or about 3 mg or about 4 mg or about 5 mg or about 7.5 mg or about 10 mg or about 16 mg, and the 2C-B tartrate Form 1 is administered between about 1 mg and 40 mg.

In some embodiments, the serotonin receptor modulator for use with the 2C-B tartrate Form 1 is an extended-release of risperidone including (RISPERDAL CONSTA), wherein the extended-release of risperidone is administered in about 12.5 mg, or about 25 mg, or about 37.5 mg, or about 50 mg, and the 2C-B tartrate Form 1 is administered between about 1 mg and 40 mg.

In some embodiments, the serotonin receptor modulator for use with the 2C-B HCl Form A is eplivanserin, wherein the eplivanserin is administered in about 1 mg to about 40 mg, or about 5 mg to about 10 mg, and the 2C-B HCl Form A is administered between about 1 mg and 40 mg.

In some embodiments, the serotonin receptor modulator for use with the 2C-B HCl Form A is volinanserin, wherein the volinanserin is administered in about 1 mg to about 60 mg, or about 5 mg to about 20 mg, and the 2C-B HCl Form A is administered between about 1 mg and 40 mg.

In some embodiments, the serotonin receptor modulator for use with the 2C-B HCl Form A is ketanserin, wherein the ketanserin is administered in about 10 mg to about 80 mg, about 30 mg to about 50 mg, or about 40 mg and the 2C-B HCl Form A is administered between about 1 mg and 40 mg.

In some embodiments, the serotonin receptor modulator for use with the 2C-B HCl Form A is ritanserin, wherein the ritanserin is administered in about 1 mg to about 40 mg, or about 2.5 mg to about 10 mg, and the 2C-B HCl Form A is administered between about 1 mg and 40 mg.

In some embodiments, the serotonin receptor modulator is pimavanserin and the 2C-B HCl Form A wherein the pimavanserin is administered in about 1 mg to about 60 mg, or about 17 mg to about 34 mg, and the 2C-B HCl Form A is administered between about 1 mg and 40 mg.

In some embodiments, the serotonin receptor modulator for use with the 2C-B HCl Form A is nelotanserin, wherein the nelotanserin is administered in about 1 mg to about 80 mg, or about 40 mg to about 80 mg, and the 2C-B HCl Form A is administered between about 1 mg and 40 mg.

In some embodiments, the serotonin receptor modulator for use with the 2C-B HCl Form A is pruvanserin, wherein the pruvanserin is administered in about 1 mg to about 40 mg, or about 3 mg to about 10 mg, and the 2C-B HCl Form A is administered between about 1 mg and 40 mg.

In some embodiments, the serotonin receptor modulator for use with the 2C-B HCl Form A is flibanserin, wherein the flibanserin is administered in about 10 mg to about 200 mg, or about 80 mg to about 120 mg, or about 100 mg and the 2C-B HCl Form A is administered between about 1 mg and 40 mg.

In some embodiments, the serotonin receptor modulator for use with the 2C-B HCl Form A is olanzapine, wherein the olanzapine is administered in about 2.5 mg to about 30 mg, or about 5 mg or about 10 mg, or about 20 mg or about 25 mg, and the 2C-B HCl Form A is administered between about 1 mg and 40 mg.

In some embodiments, the serotonin receptor modulator for use with the 2C-B HCl Form A is an extended-release of olanzapine such as ZYPREXA RELPREVV, wherein the extended release olanzapine is administered in about 50 mg to about 450 mg, or about 150 mg or about 210 mg, or about 300 mg or about 405 mg, and the 2C-B HCl Form A is administered between about 1 mg and 40 mg.

In some embodiments, the serotonin receptor modulator for use with the 2C-B HCl Form A is quetiapine, wherein the quetiapine is administered in about 25 mg to about 800 mg, or about 50 mg to about 100 mg, or about 150 mg or about 200 mg or about 250 mg or about 300 mg, and the 2C-B HCl Form A is administered between about 1 mg and 40 mg.

In some embodiments, the serotonin receptor modulator for use with the 2C-B HCl Form A is an extended-release of quetiapine, wherein the extended-release of quetiapine is administered in about 50 mg to about 300 mg, or about 50 mg or about 100 mg or about 200 mg, or about 300 mg, and the 2C-B HCl Form A is administered between about 1 mg and 40 mg.

In some embodiments, the serotonin receptor modulator for use with the 2C-B HCl Form A is risperidone, wherein the risperidone is administered in about 0.5 mg to about 20 mg or about 5 mg, or about 1 mg, or about 2 mg, or about 3 mg or about 4 mg or about 5 mg or about 7.5 mg or about 10 mg or about 16 mg, and the 2C-B HCl Form A is administered between about 1 mg and 40 mg.

In some embodiments, the serotonin receptor modulator for use with the 2C-B HCl Form A is an extended-release of risperidone including (RISPERDAL CONSTA), wherein the extended-release of risperidone is administered in about 12.5 mg, or about 25 mg, or about 37.5 mg, or about 50 mg, and the 2C-B HCl Form A is administered between about 1 mg and 40 mg.

In some embodiments, the serotonin receptor modulator for use with the 2C-B HCl Form C is eplivanserin, wherein the eplivanserin is administered in about 1 mg to about 40 mg, or about 5 mg to about 10 mg, and the 2C-B HCl Form C is administered between about 1 mg and 40 mg.

In some embodiments, the serotonin receptor modulator for use with the 2C-B HCl Form C is volinanserin, wherein the volinanserin is administered in about 1 mg to about 60 mg, or about 5 mg to about 20 mg, and the 2C-B HCl Form C is administered between about 1 mg and 40 mg.

In some embodiments, the serotonin receptor modulator for use with the 2C-B HCl Form C is ketanserin, wherein the ketanserin is administered in about 10 mg to about 80 mg, about 30 mg to about 50 mg, or about 40 mg and the 2C-B HCl Form C is administered between about 1 mg and 40 mg.

In some embodiments, the serotonin receptor modulator for use with the 2C-B HCl Form C is ritanserin, wherein the ritanserin is administered in about 1 mg to about 40 mg, or about 2.5 mg to about 10 mg, and the 2C-B HCl Form C is administered between about 1 mg and 40 mg.

In some embodiments, the serotonin receptor modulator is pimavanserin and the 2C-B HCl Form C wherein the pimavanserin is administered in about 1 mg to about 60 mg, or about 17 mg to about 34 mg, and the 2C-B HCl Form C is administered between about 1 mg and 40 mg.

In some embodiments, the serotonin receptor modulator for use with the 2C-B HCl Form C is nelotanserin, wherein the nelotanserin is administered in about 1 mg to about 80 mg, or about 40 mg to about 80 mg, and the 2C-B HCl Form C is administered between about 1 mg and 40 mg.

In some embodiments, the serotonin receptor modulator for use with the 2C-B HCl Form C is pruvanserin, wherein the pruvanserin is administered in about 1 mg to about 40 mg, or about 3 mg to about 10 mg, and the 2C-B HCl Form C is administered between about 1 mg and 40 mg.

In some embodiments, the serotonin receptor modulator for use with the 2C-B HCl Form C is flibanserin, wherein the flibanserin is administered in about 10 mg to about 200 mg, or about 80 mg to about 120 mg, or about 100 mg and the 2C-B HCl Form C is administered between about 1 mg and 40 mg.

In some embodiments, the serotonin receptor modulator for use with the 2C-B HCl Form C is olanzapine, wherein the olanzapine is administered in about 2.5 mg to about 30 mg, or about 5 mg or about 10 mg, or about 20 mg or about 25 mg, and the 2C-B HCl Form C is administered between about 1 mg and 40 mg.

In some embodiments, the serotonin receptor modulator for use with the 2C-B HCl Form C is an extended-release of olanzapine such as ZYPREXA RELPREVV, wherein the extended release olanzapine is administered in about 50 mg to about 450 mg, or about 150 mg or about 210 mg, or about 300 mg or about 405 mg, and the 2C-B HCl Form C is administered between about 1 mg and 40 mg.

In some embodiments, the serotonin receptor modulator for use with the 2C-B HCl Form C is quetiapine, wherein the quetiapine is administered in about 25 mg to about 800 mg, or about 50 mg to about 100 mg, or about 150 mg or about 200 mg or about 250 mg or about 300 mg, and the 2C-B HCl Form C is administered between about 1 mg and 40 mg.

In some embodiments, the serotonin receptor modulator for use with the 2C-B HCl Form C is an extended-release of quetiapine, wherein the extended-release of quetiapine is administered in about 50 mg to about 300 mg, or about 50 mg or about 100 mg or about 200 mg, or about 300 mg, and the 2C-B HCl Form C is administered between about 1 mg and 40 mg.

In some embodiments, the serotonin receptor modulator for use with the 2C-B HCl Form C is risperidone, wherein the risperidone is administered in about 0.5 mg to about 20 mg or about 5 mg, or about 1 mg, or about 2 mg, or about 3 mg or about 4 mg or about 5 mg or about 7.5 mg or about 10 mg or about 16 mg, and the 2C-B HCl Form C is administered between about 1 mg and 40 mg.

In some embodiments, the serotonin receptor modulator for use with the 2C-B HCl Form C is an extended-release of risperidone including (RISPERDAL CONSTA), wherein the extended-release of risperidone is administered in about 12.5 mg, or about 25 mg, or about 37.5 mg, or about 50 mg, and the 2C-B HCl Form C is administered between about 1 mg and 40 mg.

In certain embodiments, such as those described above a disclosed 2C-B or 2C-B.HCl form is co-administered with a serotonin receptor modulator in the same or in separate compositions. In one embodiment, the 2C-B or 2C-B.HCl is administered in a modified release formulation such that the subject is effectively pretreated with serotonin receptor modulator prior to release of an effective amount of the psychedelic. Thus, in some embodiments, the serotonin receptor modulator is administered or released from a composition provided herein prior to the administration and/or release of the psychedelic. This allows pretreatment to attenuate activation of the serotonin receptor by the psychedelic. In some embodiments, the serotonin receptor modulator is administered or released from the composition provided herein to pretreat a subject by at least about at about 5 minutes, 10 minutes, 20 minutes, 30 minutes, 40 minutes, 50 minutes, 1 hour, 1.25 hours, 1.5 hours, 2 hours, or 3 hours prior to the release of the psychedelic. In some embodiments, the serotonin receptor modulator attenuates the activation of the serotonin receptor when the serotonin receptor modulator is used to pretreat at most about 3 hours, 4 hours, 5 hours, 6 hours, 7 hours, 8 hours, 9 hours, or more than 9 hours prior to the release of the psychedelic. In some embodiments, the serotonin receptor modulator attenuates the activation of the serotonin receptor when the serotonin receptor modulator is used to pretreat in a range of about 5 minutes to about 3 hours, about 10 minutes to about 3 hours, about 20 minutes to about 3 hours, about 30 minutes to about 3 hours, about 40 minutes to about 3 hours, about 50 minutes to about 3 hours, about 1 hour to about 3 hours, about 5 minutes to about 2 hours, about 10 minutes to about 2 hours, about 20 minutes to about 2 hours, about 30 minutes to about 2 hours, about 40 minutes to about 2 hours, about 50 minutes to about 2 hours, about 1 hour to about 2 hours, about 5 minutes to about 1 hour, about 10 minutes to about 1 hour, about 20 minutes to about 1 hour, about 30 minutes to about 1 hour, about 40 minutes to about 1 hour, or about 50 minutes to about 1 hour prior to the release of the psychedelic.

In a preferred embodiment, the serotonin receptor modulator is administered at about 1 hour to about 3 hours prior to the administration of the psychedelic.

In some embodiments, the serotonin receptor modulator is eplivanserin and the psychedelic is 2C-B.HCl, wherein the eplivanserin is administered to pretreat at least 15 minutes prior to the administration of 2C-B HCl. In some embodiments, the serotonin receptor modulator is eplivanserin and the psychedelic is 2C-B.HCl, wherein the eplivanserin is administered to pretreat between at least 30 minutes prior and 360 minutes prior to the release or administration of the 2C-B.HCl. In some embodiments, the serotonin receptor modulator is eplivanserin and the psychedelic is 2C-B.HCl, wherein the eplivanserin is administered to pretreat between at least 60 minutes prior and 360 minutes prior to the release or administration the 2C-B.HCl. In some embodiments, the serotonin receptor modulator is eplivanserin and the psychedelic is 2C-B.HCl, wherein the eplivanserin is administered to pretreat at between least 90 minutes and 240 minutes prior to the 2C-B.HCl. In some embodiments, the serotonin receptor modulator is eplivanserin and the psychedelic is 2C-B.HCl, wherein the eplivanserin is administered to pretreat at least 120 minutes prior to the 2C-B.HCl

In some embodiments, the serotonin receptor modulator is eplivanserin and the psychedelic is 2C-B.HCl, wherein the eplivanserin is administered to pretreat at least 150 minutes prior to the 2C-B HCl. In some embodiments, the serotonin receptor modulator is eplivanserin and the psychedelic is 2C-B.HCl, wherein the eplivanserin is administered to pretreat at least 180 minutes prior to the 2C-B.HCl. In some embodiments, the serotonin receptor modulator is eplivanserin and the psychedelic is 2C-B.HCl, wherein the eplivanserin is administered to pretreat at least 210 minutes prior to the 2C-B.HCl. In some embodiments, the serotonin receptor modulator is eplivanserin and the psychedelic is 2C-B.HCl, wherein the eplivanserin is administered to pretreat at least 240 minutes prior to the 2C-B.HCl

In some embodiments, the serotonin receptor modulator is eplivanserin and the psychedelic is 2C-B.HCl, wherein the eplivanserin is administered to pretreat at least 270 minutes prior to 2C-B.HCl. In some embodiments, the serotonin receptor modulator is eplivanserin and the psychedelic is 2C-B.HCl, wherein the eplivanserin is administered to pretreat at least 300 minutes prior to the 2C-B.HCl. In some embodiments, the serotonin receptor modulator is eplivanserin and the psychedelic is 2C-B.HCl, wherein the eplivanserin is administered to pretreat at least 330 minutes prior to the 2C-B.HCl. In some embodiments, the serotonin receptor modulator is eplivanserin and the psychedelic is 2C-B.HCl, wherein the eplivanserin is administered to pretreat at least 360 minutes prior to the 2C-B HCl.

In some preferred embodiments, the serotonin receptor modulator is eplivanserin and the psychedelic is 2C-B.HCl, wherein eplivanserin is administered to pretreat between about 60 minutes and about 180 minutes prior to the administration of 2C-B.HCl

In some embodiments, the serotonin receptor modulator is volinanserin and the psychedelic is 2C-B.HCl, wherein the volinanserin is administered to pretreat a subject between at least 15 minutes and 360 minutes prior to the administration or release of 2C-B HCl. In some embodiments, the serotonin receptor modulator is volinanserin and the psychedelic is 2C-B.HCl, wherein the volinanserin is administered to pretreat between at least 30 minutes and 360 minutes prior to the administration or release of 2C-B.HCl. In some embodiments, the serotonin receptor modulator is volinanserin and the psychedelic is 2C-B HCl, wherein the volinanserin is administered to pretreat between at least 60 minutes and 240 minutes prior to the administration or release of 2C-B.HCl. In some embodiments, the serotonin receptor modulator is volinanserin and the psychedelic is 2C-B.HCl, wherein the volinanserin is administered to pretreat at least 90 minutes prior to 2C-B.HCl. In some embodiments, the serotonin receptor modulator is volinanserin and the psychedelic is 2C-B HCl, wherein the volinanserin is administered to pretreat at least 120 minutes prior to the 2C-B.HCl

In some embodiments, the serotonin receptor modulator is volinanserin and the psychedelic is 2C-B HCl, wherein the volinanserin is administered to pretreat at least 150 minutes prior to the 2C-B HCl. In some embodiments, the serotonin receptor modulator is volinanserin and the psychedelic is 2C-B.HCl, wherein the volinanserin is administered to pretreat between about 15 minutes and about 150 minutes prior to the 2C-B.HCl. In some embodiments, the serotonin receptor modulator is volinanserin and the psychedelic is 2C-B.HCl, wherein the volinanserin is administered to pretreat at least 180 minutes prior to the 2C-B HCl. In some embodiments, the serotonin receptor modulator is volinanserin and the psychedelic is 2C-B.HCl, wherein the volinanserin is administered to pretreat at least 210 minutes prior to the 2C-B.HCl. In some embodiments, the serotonin receptor modulator is volinanserin and the psychedelic is 2C-B.HCl, wherein the volinanserin is administered to pretreat at least 240 minutes prior to the 2C-B HCl. In some embodiments, the serotonin receptor modulator is volinanserin and the psychedelic is 2C-B.HCl, wherein the volinanserin is administered to pretreat at least 270 minutes prior to the 2C-B.HCl. In some embodiments, the serotonin receptor modulator is volinanserin and the psychedelic is 2C-B.HCl, wherein the volinanserin is administered to pretreat at least 300 minutes prior to the 2C-B.HCl. In some embodiments, the serotonin receptor modulator is volinanserin and the psychedelic is 2C-B.HCl, wherein the volinanserin is administered to pretreat at least 330 minutes prior to the 2C-B.HCl. In some embodiments, the serotonin receptor modulator is volinanserin and the psychedelic is 2C-B.HCl, wherein the volinanserin is administered to pretreat at least 360 minutes prior to the 2C-B.HCl. In some preferred embodiments, the serotonin receptor modulator is volinanserin and the psychedelic is 2C-B.HCl, wherein volinanserin is administered to pretreat between about 60 minutes and about 180 minutes prior to the administration of 2C-B.HCl

In some embodiments, the serotonin receptor modulator is ketanserin and the psychedelic is 2C-B.HCl, wherein the ketanserin is administered to pretreat at least 15 minutes prior to the administration of 2C-B.HCl. In some embodiments, the serotonin receptor modulator is ketanserin and the psychedelic is 2C-B.HCl, wherein the ketanserin is administered to pretreat between at least 30 minutes and 360 minutes prior to the administration or release 2C-B HCl. In some embodiments, the serotonin receptor modulator is ketanserin and the psychedelic is 2C-B.HCl, wherein the ketanserin is administered to pretreat between at least 60 minutes and 240 minutes prior to the administration or release of 2C-B.HCl. In some embodiments, the serotonin receptor modulator is ketanserin and the psychedelic is 2C-B.HCl, wherein the ketanserin is administered to pretreat at least 90 minutes prior to the 2C-B.HCl. In some embodiments, the serotonin receptor modulator is ketanserin and the psychedelic is 2C-B.HCl, wherein the ketanserin is administered to pretreat at least 120 minutes prior to the 2C-B.HCl. In some embodiments, the serotonin receptor modulator is ketanserin and the psychedelic is 2C-B.HCl, wherein the ketanserin is administered to pretreat at least 150 minutes prior to the 2C-B.HCl. In some embodiments, the serotonin receptor modulator is ketanserin and the psychedelic is 2C-B.HCl, wherein the ketanserin is administered to pretreat between about 15 minutes and about 150 minutes prior to the 2C-B.HCl. In some embodiments, the serotonin receptor modulator is ketanserin and the psychedelic is 2C-B.HCl, wherein the ketanserin is administered to pretreat at least 180 minutes prior to the 2C-B HCl. In some embodiments, the serotonin receptor modulator is ketanserin and the psychedelic is 2C-B.HCl, wherein the ketanserin is administered to pretreat at least 210 minutes prior to the 2C-B.HCl. In some embodiments, the serotonin receptor modulator is ketanserin and the psychedelic is 2C-B.HCl, wherein the ketanserin is administered to pretreat at least 240 minutes prior to the 2C-B.HCl. In some embodiments, the serotonin receptor modulator is ketanserin and the psychedelic is 2C-B.HCl, wherein the ketanserin is administered to pretreat at least 270 minutes prior to the 2C-B.HCl. In some embodiments, the serotonin receptor modulator is ketanserin and the psychedelic is 2C-B.HCl, wherein the ketanserin is administered to pretreat at least 300 minutes prior to the 2C-B.HCl. In some embodiments, the serotonin receptor modulator is ketanserin and the psychedelic is 2C-B.HCl, wherein the ketanserin is administered to pretreat at least 330 minutes prior to the 2C-B.HCl. In some embodiments, the serotonin receptor modulator is ketanserin and the psychedelic is 2C-B.HCl, wherein the ketanserin is administered to pretreat at least 360 minutes prior to the 2C-B.HCl. In some preferred embodiments, the serotonin receptor modulator is ketanserin and the psychedelic is 2C-B.HCl, wherein ketanserin is administered to pretreat between about 60 minutes and about 180 minutes prior to the administration of 2C-B.HCl

In some embodiments, the serotonin receptor modulator is ritanserin and the psychedelic is 2C-B.HCl, wherein the ritanserin is administered to pretreat at least 15 minutes prior to the administration of 2C-B.HCl. In some embodiments, the serotonin receptor modulator is ritanserin and the psychedelic is 2C-B.HCl, wherein the ritanserin is administered to pretreat at least 30 minutes prior to the 2C-B.HCl. In some embodiments, the serotonin receptor modulator is ritanserin and the psychedelic is 2C-B.HCl, wherein the ritanserin is administered to pretreat between at least 60 minutes and 240 minutes prior to the administration or release of 2C-B HCl. In some embodiments, the serotonin receptor modulator is ritanserin and the psychedelic is 2C-B.HCl, wherein the ritanserin is administered to pretreat at least 90 minutes prior to the 2C-B.HCl. In some embodiments, the serotonin receptor modulator is ritanserin and the psychedelic is 2C-B.HCl, wherein the ritanserin is administered to pretreat at least 120 minutes prior to the 2C-B.HCl. In some embodiments, the serotonin receptor modulator is ritanserin and the psychedelic is 2C-B.HCl, wherein the ritanserin is administered to pretreat at least 150 minutes prior to the 2C-B.HCl. In some embodiments, the serotonin receptor modulator is ritanserin and the psychedelic is 2C-B.HCl, wherein the ritanserin is administered to pretreat between about 15 minutes and about 150 minutes prior to the 2C-B HCl. In some embodiments, the serotonin receptor modulator is ritanserin and the psychedelic is 2C-B.HCl, wherein the ritanserin is administered to pretreat at least 180 minutes prior to the 2C-B.HCl. In some embodiments, the serotonin receptor modulator is ritanserin and the psychedelic is 2C-B.HCl, wherein the ritanserin is administered to pretreat at least 210 minutes prior to the 2C-B.HCl. In some embodiments, the serotonin receptor modulator is ritanserin and the psychedelic is 2C-B.HCl, wherein the ritanserin is administered to pretreat at least 240 minutes prior to the 2C-B.HCl. In some embodiments, the serotonin receptor modulator is ritanserin and the psychedelic is 2C-B.HCl, wherein the ritanserin is administered to pretreat at least 270 minutes prior to the 2C-B.HCl. In some embodiments, the serotonin receptor modulator is ritanserin and the psychedelic is 2C-B.HCl, wherein the ritanserin is administered to pretreat at least 300 minutes prior to the 2C-B.HCl. In some embodiments, the serotonin receptor modulator is ritanserin and the psychedelic is 2C-B.HCl, wherein the ritanserin is administered to pretreat at least 330 minutes prior to the 2C-B.HCl. In some embodiments, the serotonin receptor modulator is ritanserin and the psychedelic is 2C-B.HCl, wherein the ritanserin is administered to pretreat at least 360 minutes prior to the 2C-B.HCl. In some preferred embodiments, the serotonin receptor modulator is ritanserin and the psychedelic is 2C-B.HCl, wherein ritanserin is administered to pretreat between about 60 minutes and about 180 minutes prior to the administration of 2C-B.HCl.

In some embodiments, the serotonin receptor modulator is pimavanserin and the psychedelic is 2C-B.HCl, wherein the pimavanserin is administered to pretreat at least 15 minutes prior to the administration of 2C-B.HCl. In some embodiments, the serotonin receptor modulator is pimavanserin and the psychedelic is 2C-B.HCl, wherein the pimavanserin is administered to pretreat at least 30 minutes prior to the 2C-B.HCl. In some embodiments, the serotonin receptor modulator is pimavanserin and the psychedelic is 2C-B.HCl, wherein the pimavanserin is administered to pretreat between at least 60 minutes and 240 minutes prior to the administration or release of 2C-B.HCl. In some embodiments, the serotonin receptor modulator is pimavanserin and the psychedelic is 2C-B.HCl, wherein the pimavanserin is administered to pretreat at least 90 minutes prior to the 2C-B.HCl. In some embodiments, the serotonin receptor modulator is pimavanserin and the psychedelic is 2C-B.HCl, wherein the pimavanserin is administered to pretreat at least 120 minutes prior to the 2C-B.HCl. In some embodiments, the serotonin receptor modulator is pimavanserin and the psychedelic is 2C-B.HCl, wherein the pimavanserin is administered to pretreat at least 150 minutes prior to the 2C-B.HCl. In some embodiments, the serotonin receptor modulator is pimavanserin and the psychedelic is 2C-B.HCl, wherein the pimavanserin is administered to pretreat between about 15 minutes and about 150 minutes prior to the 2C-B HCl. In some embodiments, the serotonin receptor modulator is pimavanserin and the psychedelic is 2C-B.HCl, wherein the pimavanserin is administered to pretreat at least 180 minutes prior to the 2C-B.HCl. In some embodiments, the serotonin receptor modulator is pimavanserin and the psychedelic is 2C-B.HCl, wherein the pimavanserin is administered to pretreat at least 210 minutes prior to the 2C-B.HCl. In some embodiments, the serotonin receptor modulator is pimavanserin and the psychedelic is 2C-B.HCl, wherein the pimavanserin is administered to pretreat at least 240 minutes prior to the 2C-B.HCl. In some embodiments, the serotonin receptor modulator is pimavanserin and the psychedelic is 2C-B.HCl, wherein the pimavanserin is administered to pretreat at least 270 minutes prior to the 2C-B HCl. In some embodiments, the serotonin receptor modulator is pimavanserin and the psychedelic is 2C-B HCl, wherein the pimavanserin is administered to pretreat at least 300 minutes prior to the 2C-B.HCl. In some embodiments, the serotonin receptor modulator is pimavanserin and the psychedelic is 2C-B.HCl, wherein the pimavanserin is administered to pretreat at least 330 minutes prior to the 2C-B.HCl. In some embodiments, the serotonin receptor modulator is pimavanserin and the psychedelic is 2C-B.HCl, wherein the pimavanserin is administered to pretreat at least 360 minutes prior to the 2C-B.HCl. In some preferred embodiments, the serotonin receptor modulator is pimavanserin and the psychedelic is 2C-B.HCl, wherein pimavanserin is administered to pretreat between about 60 minutes and about 180 minutes prior to the administration of 2C-B.HCl

In some embodiments, the serotonin receptor modulator is nelotanserin and the psychedelic is 2C-B.HCl, wherein the nelotanserin is administered to pretreat at least 15 minutes prior to the administration of 2C-B HCl. In some embodiments, the serotonin receptor modulator is nelotanserin and the psychedelic is 2C-B.HCl, wherein the nelotanserin is administered to pretreat at least 30 minutes prior to the 2C-B.HCl. In some embodiments, the serotonin receptor modulator is nelotanserin and the psychedelic is 2C-B.HCl, wherein the nelotanserin is administered to pretreat between at least 60 minutes and 240 minutes prior to the administration or release of 2C-B.HCl. In some embodiments, the serotonin receptor modulator is nelotanserin and the psychedelic is 2C-B.HCl, wherein the nelotanserin is administered to pretreat at least 90 minutes prior to the 2C-B.HCl. In some embodiments, the serotonin receptor modulator is nelotanserin and the psychedelic is 2C-B.HCl, wherein the nelotanserin is administered to pretreat at least 120 minutes prior to the 2C-B.HCl.

In some embodiments, the serotonin receptor modulator is nelotanserin and the psychedelic is 2C-B HCl, wherein the nelotanserin is administered to pretreat at least 150 minutes prior to the 2C-B HCl. In some embodiments, the serotonin receptor modulator is nelotanserin and the psychedelic is 2C-B.HCl, wherein the nelotanserin is administered to pretreat between about 15 minutes and about 150 minutes prior to the 2C-B.HCl. In some embodiments, the serotonin receptor modulator is nelotanserin and the psychedelic is 2C-B.HCl, wherein the nelotanserin is administered to pretreat at least 180 minutes prior to the 2C-B.HCl. In some embodiments, the serotonin receptor modulator is nelotanserin and the psychedelic is 2C-B.HCl, wherein the nelotanserin is administered to pretreat at least 210 minutes prior to the 2C-B.HCl. In some embodiments, the serotonin receptor modulator is nelotanserin and the psychedelic is 2C-B.HCl, wherein the nelotanserin is administered to pretreat at least 240 minutes prior to the 2C-B HCl. In some embodiments, the serotonin receptor modulator is nelotanserin and the psychedelic is 2C-B.HCl, wherein the nelotanserin is administered to pretreat at least 270 minutes prior to the 2C-B.HCl. In some embodiments, the serotonin receptor modulator is nelotanserin and the psychedelic is 2C-B.HCl, wherein the nelotanserin is administered to pretreat at least 300 minutes prior to the 2C-B.HCl. In some embodiments, the serotonin receptor modulator is nelotanserin and the psychedelic is 2C-B.HCl, wherein the nelotanserin is administered to pretreat at least 330 minutes prior to the 2C-B.HCl. In some embodiments, the serotonin receptor modulator is nelotanserin and the psychedelic is 2C-B.HCl, wherein the nelotanserin is administered to pretreat at least 360 minutes prior to the 2C-B.HCl. In some preferred embodiments, the serotonin receptor modulator is nelotanserin and the psychedelic is 2C-B.HCl, wherein nelotanserin is administered to pretreat between about 60 minutes and about 180 minutes prior to the administration of 2C-B.HCl

In some embodiments, the serotonin receptor modulator is pruvanserin and the psychedelic is 2C-B.HCl, wherein the pruvanserin is administered to pretreat at least 15 minutes prior to the administration of 2C-B HCl. In some embodiments, the serotonin receptor modulator is pruvanserin and the psychedelic is 2C-B.HCl, wherein the pruvanserin is administered to pretreat at least 30 minutes prior to the 2C-B.HCl. In some embodiments, the serotonin receptor modulator is pruvanserin and the psychedelic is 2C-B.HCl, wherein the pruvanserin is administered to pretreat between at least 60 minutes and 240 minutes prior to the administration or release of 2C-B.HCl. In some embodiments, the serotonin receptor modulator is pruvanserin and the psychedelic is 2C-B.HCl, wherein the pruvanserin is administered to pretreat at least 90 minutes prior to the 2C-B.HCl. In some embodiments, the serotonin receptor modulator is pruvanserin and the psychedelic is 2C-B.HCl, wherein the pruvanserin is administered to pretreat at least 120 minutes prior to the 2C-B.HCl. In some embodiments, the serotonin receptor modulator is pruvanserin and the psychedelic is 2C-B.HCl, wherein the pruvanserin is administered to pretreat at least 150 minutes prior to the 2C-B HCl. In some embodiments, the serotonin receptor modulator is pruvanserin and the psychedelic is 2C-B.HCl, wherein the pruvanserin is administered to pretreat between about 15 minutes and about 150 minutes prior to the 2C-B HCl. In some embodiments, the serotonin receptor modulator is pruvanserin and the psychedelic is 2C-B.HCl, wherein the pruvanserin is administered to pretreat at least 180 minutes prior to the 2C-B.HCl. In some embodiments, the serotonin receptor modulator is pruvanserin and the psychedelic is 2C-B.HCl, wherein the pruvanserin is administered to pretreat at least 210 minutes prior to the 2C-B.HCl

In some embodiments, the serotonin receptor modulator is pruvanserin and the psychedelic is 2C-B.HCl, wherein the pruvanserin is administered to pretreat at least 240 minutes prior to the 2C-B HCl. In some embodiments, the serotonin receptor modulator is pruvanserin and the psychedelic is 2C-B.HCl, wherein the pruvanserin is administered to pretreat at least 270 minutes prior to the 2C-B.HCl. In some embodiments, the serotonin receptor modulator is pruvanserin and the psychedelic is 2C-B.HCl, wherein the pruvanserin is administered to pretreat at least 300 minutes prior to the 2C-B.HCl. In some embodiments, the serotonin receptor modulator is pruvanserin and the psychedelic is 2C-B.HCl, wherein the pruvanserin is administered to pretreat at least 330 minutes prior to the 2C-B.HCl. In some embodiments, the serotonin receptor modulator is pruvanserin and the psychedelic is 2C-B.HCl, wherein the pruvanserin is administered to pretreat at least 360 minutes prior to the 2C-B.HCl. In some preferred embodiments, the serotonin receptor modulator is pruvanserin and the psychedelic is 2C-B.HCl, wherein pruvanserin is administered to pretreat between about 60 minutes and about 180 minutes prior to the administration of 2C-B.HCl

In some embodiments, the serotonin receptor modulator is eplivanserin and the psychedelic is 2C-B, wherein the eplivanserin is administered to pretreat at least 15 minutes prior to the administration of 2C-B. In some embodiments, the serotonin receptor modulator is eplivanserin and the psychedelic is 2C-B, wherein the eplivanserin is administered to pretreat between at least 30 minutes prior and 360 minutes prior to the release or administration of the 2C-B. In some embodiments, the serotonin receptor modulator is eplivanserin and the psychedelic is 2C-B, wherein the eplivanserin is administered to pretreat between at least 60 minutes prior and 360 minutes prior to the release or administration the 2C-B. In some embodiments, the serotonin receptor modulator is eplivanserin and the psychedelic is 2C-B, wherein the eplivanserin is administered to pretreat at between least 90 minutes and 240 minutes prior to the 2C-B. In some embodiments, the serotonin receptor modulator is eplivanserin and the psychedelic is 2C-B, wherein the eplivanserin is administered to pretreat at least 120 minutes prior to the 2C-B.

In some embodiments, the serotonin receptor modulator is eplivanserin and the psychedelic is 2C-B, wherein the eplivanserin is administered to pretreat at least 150 minutes prior to the 2C-B. In some embodiments, the serotonin receptor modulator is eplivanserin and the psychedelic is 2C-B, wherein the eplivanserin is administered to pretreat at least 180 minutes prior to the 2C-B. In some embodiments, the serotonin receptor modulator is eplivanserin and the psychedelic is 2C-B, wherein the eplivanserin is administered to pretreat at least 210 minutes prior to the 2C-B. In some embodiments, the serotonin receptor modulator is eplivanserin and the psychedelic is 2C-B, wherein the eplivanserin is administered to pretreat at least 240 minutes prior to the 2C-B.

In some embodiments, the serotonin receptor modulator is eplivanserin and the psychedelic is 2C-B, wherein the eplivanserin is administered to pretreat at least 270 minutes prior to 2C-B. In some embodiments, the serotonin receptor modulator is eplivanserin and the psychedelic is 2C-B, wherein the eplivanserin is administered to pretreat at least 300 minutes prior to the 2C-B. In some embodiments, the serotonin receptor modulator is eplivanserin and the psychedelic is 2C-B, wherein the eplivanserin is administered to pretreat at least 330 minutes prior to the 2C-B. In some embodiments, the serotonin receptor modulator is eplivanserin and the psychedelic is 2C-B, wherein the eplivanserin is administered to pretreat at least 360 minutes prior to the 2C-B.

In some preferred embodiments, the serotonin receptor modulator is eplivanserin and the psychedelic is 2C-B, wherein eplivanserin is administered to pretreat between about 60 minutes and about 180 minutes prior to the administration of 2C-B.

In some embodiments, the serotonin receptor modulator is volinanserin and the psychedelic is 2C-B, wherein the volinanserin is administered to pretreat a subject between at least 15 minutes and 360 minutes prior to the administration or release of 2C-B. In some embodiments, the serotonin receptor modulator is volinanserin and the psychedelic is 2C-B, wherein the volinanserin is administered to pretreat between at least 30 minutes and 360 minutes prior to the administration or release of 2C-B. In some embodiments, the serotonin receptor modulator is volinanserin and the psychedelic is 2C-B, wherein the volinanserin is administered to pretreat between at least 60 minutes and 240 minutes prior to the administration or release of 2C-B. In some embodiments, the serotonin receptor modulator is volinanserin and the psychedelic is 2C-B, wherein the volinanserin is administered to pretreat at least 90 minutes prior to 2C-B. In some embodiments, the serotonin receptor modulator is volinanserin and the psychedelic is 2C-B, wherein the volinanserin is administered to pretreat at least 120 minutes prior to the 2C-B.

In some embodiments, the serotonin receptor modulator is volinanserin and the psychedelic is 2C-B, wherein the volinanserin is administered to pretreat at least 150 minutes prior to the 2C-B. In some embodiments, the serotonin receptor modulator is volinanserin and the psychedelic is 2C-B, wherein the volinanserin is administered to pretreat between about 15 minutes and about 150 minutes prior to the 2C-B. In some embodiments, the serotonin receptor modulator is volinanserin and the psychedelic is 2C-B, wherein the volinanserin is administered to pretreat at least 180 minutes prior to the 2C-B. In some embodiments, the serotonin receptor modulator is volinanserin and the psychedelic is 2C-B, wherein the volinanserin is administered to pretreat at least 210 minutes prior to the 2C-B. In some embodiments, the serotonin receptor modulator is volinanserin and the psychedelic is 2C-B, wherein the volinanserin is administered to pretreat at least 240 minutes prior to the 2C-B. In some embodiments, the serotonin receptor modulator is volinanserin and the psychedelic is 2C-B, wherein the volinanserin is administered to pretreat at least 270 minutes prior to the 2C-B. In some embodiments, the serotonin receptor modulator is volinanserin and the psychedelic is 2C-B, wherein the volinanserin is administered to pretreat at least 300 minutes prior to the 2C-B. In some embodiments, the serotonin receptor modulator is volinanserin and the psychedelic is 2C-B, wherein the volinanserin is administered to pretreat at least 330 minutes prior to the 2C-B. In some embodiments, the serotonin receptor modulator is volinanserin and the psychedelic is 2C-B, wherein the volinanserin is administered to pretreat at least 360 minutes prior to the 2C-B. In some preferred embodiments, the serotonin receptor modulator is volinanserin and the psychedelic is 2C-B, wherein volinanserin is administered to pretreat between about 60 minutes and about 180 minutes prior to the administration of 2C-B.

In some embodiments, the serotonin receptor modulator is ketanserin and the psychedelic is 2C-B, wherein the ketanserin is administered to pretreat at least 15 minutes prior to the administration of 2C-B. In some embodiments, the serotonin receptor modulator is ketanserin and the psychedelic is 2C-B, wherein the ketanserin is administered to pretreat between at least 30 minutes and 360 minutes prior to the administration or release 2C-B. In some embodiments, the serotonin receptor modulator is ketanserin and the psychedelic is 2C-B, wherein the ketanserin is administered to pretreat between at least 60 minutes and 240 minutes prior to the administration or release of 2C-B. In some embodiments, the serotonin receptor modulator is ketanserin and the psychedelic is 2C-B, wherein the ketanserin is administered to pretreat at least 90 minutes prior to the 2C-B. In some embodiments, the serotonin receptor modulator is ketanserin and the psychedelic is 2C-B, wherein the ketanserin is administered to pretreat at least 120 minutes prior to the 2C-B. In some embodiments, the serotonin receptor modulator is ketanserin and the psychedelic is 2C-B, wherein the ketanserin is administered to pretreat at least 150 minutes prior to the 2C-B. In some embodiments, the serotonin receptor modulator is ketanserin and the psychedelic is 2C-B, wherein the ketanserin is administered to pretreat between about 15 minutes and about 150 minutes prior to the 2C-B. In some embodiments, the serotonin receptor modulator is ketanserin and the psychedelic is 2C-B, wherein the ketanserin is administered to pretreat at least 180 minutes prior to the 2C-B. In some embodiments, the serotonin receptor modulator is ketanserin and the psychedelic is 2C-B, wherein the ketanserin is administered to pretreat at least 210 minutes prior to the 2C-B. In some embodiments, the serotonin receptor modulator is ketanserin and the psychedelic is 2C-B, wherein the ketanserin is administered to pretreat at least 240 minutes prior to the 2C-B. In some embodiments, the serotonin receptor modulator is ketanserin and the psychedelic is 2C-B, wherein the ketanserin is administered to pretreat at least 270 minutes prior to the 2C-B. In some embodiments, the serotonin receptor modulator is ketanserin and the psychedelic is 2C-B, wherein the ketanserin is administered to pretreat at least 300 minutes prior to the 2C-B. In some embodiments, the serotonin receptor modulator is ketanserin and the psychedelic is 2C-B, wherein the ketanserin is administered to pretreat at least 330 minutes prior to the 2C-B. In some embodiments, the serotonin receptor modulator is ketanserin and the psychedelic is 2C-B, wherein the ketanserin is administered to pretreat at least 360 minutes prior to the 2C-B. In some preferred embodiments, the serotonin receptor modulator is ketanserin and the psychedelic is 2C-B, wherein ketanserin is administered to pretreat between about 60 minutes and about 180 minutes prior to the administration of 2C-B.

In some embodiments, the serotonin receptor modulator is ritanserin and the psychedelic is 2C-B, wherein the ritanserin is administered to pretreat at least 15 minutes prior to the administration of 2C-B. In some embodiments, the serotonin receptor modulator is ritanserin and the psychedelic is 2C-B, wherein the ritanserin is administered to pretreat at least 30 minutes prior to the 2C-B. In some embodiments, the serotonin receptor modulator is ritanserin and the psychedelic is 2C-B, wherein the ritanserin is administered to pretreat between at least 60 minutes and 240 minutes prior to the administration or release of 2C-B. In some embodiments, the serotonin receptor modulator is ritanserin and the psychedelic is 2C-B, wherein the ritanserin is administered to pretreat at least 90 minutes prior to the 2C-B. In some embodiments, the serotonin receptor modulator is ritanserin and the psychedelic is 2C-B, wherein the ritanserin is administered to pretreat at least 120 minutes prior to the 2C-B. In some embodiments, the serotonin receptor modulator is ritanserin and the psychedelic is 2C-B, wherein the ritanserin is administered to pretreat at least 150 minutes prior to the 2C-B. In some embodiments, the serotonin receptor modulator is ritanserin and the psychedelic is 2C-B, wherein the ritanserin is administered to pretreat between about 15 minutes and about 150 minutes prior to the 2C-B. In some embodiments, the serotonin receptor modulator is ritanserin and the psychedelic is 2C-B, wherein the ritanserin is administered to pretreat at least 180 minutes prior to the 2C-B. In some embodiments, the serotonin receptor modulator is ritanserin and the psychedelic is 2C-B, wherein the ritanserin is administered to pretreat at least 210 minutes prior to the 2C-B. In some embodiments, the serotonin receptor modulator is ritanserin and the psychedelic is 2C-B, wherein the ritanserin is administered to pretreat at least 240 minutes prior to the 2C-B. In some embodiments, the serotonin receptor modulator is ritanserin and the psychedelic is 2C-B, wherein the ritanserin is administered to pretreat at least 270 minutes prior to the 2C-B. In some embodiments, the serotonin receptor modulator is ritanserin and the psychedelic is 2C-B, wherein the ritanserin is administered to pretreat at least 300 minutes prior to the 2C-B. In some embodiments, the serotonin receptor modulator is ritanserin and the psychedelic is 2C-B, wherein the ritanserin is administered to pretreat at least 330 minutes prior to the 2C-B. In some embodiments, the serotonin receptor modulator is ritanserin and the psychedelic is 2C-B, wherein the ritanserin is administered to pretreat at least 360 minutes prior to the 2C-B. In some preferred embodiments, the serotonin receptor modulator is ritanserin and the psychedelic is 2C-B, wherein ritanserin is administered to pretreat between about 60 minutes and about 180 minutes prior to the administration of 2C-B.

In some embodiments, the serotonin receptor modulator is pimavanserin and the psychedelic is 2C-B, wherein the pimavanserin is administered to pretreat at least 15 minutes prior to the administration of 2C-B. In some embodiments, the serotonin receptor modulator is pimavanserin and the psychedelic is 2C-B, wherein the pimavanserin is administered to pretreat at least 30 minutes prior to the 2C-B. In some embodiments, the serotonin receptor modulator is pimavanserin and the psychedelic is 2C-B, wherein the pimavanserin is administered to pretreat between at least 60 minutes and 240 minutes prior to the administration or release of 2C-B. In some embodiments, the serotonin receptor modulator is pimavanserin and the psychedelic is 2C-B, wherein the pimavanserin is administered to pretreat at least 90 minutes prior to the 2C-B. In some embodiments, the serotonin receptor modulator is pimavanserin and the psychedelic is 2C-B, wherein the pimavanserin is administered to pretreat at least 120 minutes prior to the 2C-B. In some embodiments, the serotonin receptor modulator is pimavanserin and the psychedelic is 2C-B, wherein the pimavanserin is administered to pretreat at least 150 minutes prior to the 2C-B. In some embodiments, the serotonin receptor modulator is pimavanserin and the psychedelic is 2C-B, wherein the pimavanserin is administered to pretreat between about 15 minutes and about 150 minutes prior to the 2C-B. In some embodiments, the serotonin receptor modulator is pimavanserin and the psychedelic is 2C-B, wherein the pimavanserin is administered to pretreat at least 180 minutes prior to the 2C-B. In some embodiments, the serotonin receptor modulator is pimavanserin and the psychedelic is 2C-B, wherein the pimavanserin is administered to pretreat at least 210 minutes prior to the 2C-B. In some embodiments, the serotonin receptor modulator is pimavanserin and the psychedelic is 2C-B, wherein the pimavanserin is administered to pretreat at least 240 minutes prior to the 2C-B. In some embodiments, the serotonin receptor modulator is pimavanserin and the psychedelic is 2C-B, wherein the pimavanserin is administered to pretreat at least 270 minutes prior to the 2C-B. In some embodiments, the serotonin receptor modulator is pimavanserin and the psychedelic is 2C-B, wherein the pimavanserin is administered to pretreat at least 300 minutes prior to the 2C-B. In some embodiments, the serotonin receptor modulator is pimavanserin and the psychedelic is 2C-B, wherein the pimavanserin is administered to pretreat at least 330 minutes prior to the 2C-B. In some embodiments, the serotonin receptor modulator is pimavanserin and the psychedelic is 2C-B, wherein the pimavanserin is administered to pretreat at least 360 minutes prior to the 2C-B. In some preferred embodiments, the serotonin receptor modulator is pimavanserin and the psychedelic is 2C-B, wherein pimavanserin is administered to pretreat between about 60 minutes and about 180 minutes prior to the administration of 2C-B.

In some embodiments, the serotonin receptor modulator is nelotanserin and the psychedelic is 2C-B, wherein the nelotanserin is administered to pretreat at least 15 minutes prior to the administration of 2C-B. In some embodiments, the serotonin receptor modulator is nelotanserin and the psychedelic is 2C-B, wherein the nelotanserin is administered to pretreat at least 30 minutes prior to the 2C-B. In some embodiments, the serotonin receptor modulator is nelotanserin and the psychedelic is 2C-B, wherein the nelotanserin is administered to pretreat between at least 60 minutes and 240 minutes prior to the administration or release of 2C-B. In some embodiments, the serotonin receptor modulator is nelotanserin and the psychedelic is 2C-B, wherein the nelotanserin is administered to pretreat at least 90 minutes prior to the 2C-B. In some embodiments, the serotonin receptor modulator is nelotanserin and the psychedelic is 2C-B, wherein the nelotanserin is administered to pretreat at least 120 minutes prior to the 2C-B.

In some embodiments, the serotonin receptor modulator is nelotanserin and the psychedelic is 2C-B, wherein the nelotanserin is administered to pretreat at least 150 minutes prior to the 2C-B. In some embodiments, the serotonin receptor modulator is nelotanserin and the psychedelic is 2C-B, wherein the nelotanserin is administered to pretreat between about 15 minutes and about 150 minutes prior to the 2C-B. In some embodiments, the serotonin receptor modulator is nelotanserin and the psychedelic is 2C-B, wherein the nelotanserin is administered to pretreat at least 180 minutes prior to the 2C-B. In some embodiments, the serotonin receptor modulator is nelotanserin and the psychedelic is 2C-B, wherein the nelotanserin is administered to pretreat at least 210 minutes prior to the 2C-B. In some embodiments, the serotonin receptor modulator is nelotanserin and the psychedelic is 2C-B, wherein the nelotanserin is administered to pretreat at least 240 minutes prior to the 2C-B. In some embodiments, the serotonin receptor modulator is nelotanserin and the psychedelic is 2C-B, wherein the nelotanserin is administered to pretreat at least 270 minutes prior to the 2C-B. In some embodiments, the serotonin receptor modulator is nelotanserin and the psychedelic is 2C-B, wherein the nelotanserin is administered to pretreat at least 300 minutes prior to the 2C-B. In some embodiments, the serotonin receptor modulator is nelotanserin and the psychedelic is 2C-B, wherein the nelotanserin is administered to pretreat at least 330 minutes prior to the 2C-B. In some embodiments, the serotonin receptor modulator is nelotanserin and the psychedelic is 2C-B, wherein the nelotanserin is administered to pretreat at least 360 minutes prior to the 2C-B. In some preferred embodiments, the serotonin receptor modulator is nelotanserin and the psychedelic is 2C-B, wherein nelotanserin is administered to pretreat between about 60 minutes and about 180 minutes prior to the administration of 2C-B.

In some embodiments, the serotonin receptor modulator is pruvanserin and the psychedelic is 2C-B, wherein the pruvanserin is administered to pretreat at least 15 minutes prior to the administration of 2C-B. In some embodiments, the serotonin receptor modulator is pruvanserin and the psychedelic is 2C-B, wherein the pruvanserin is administered to pretreat at least 30 minutes prior to the 2C-B. In some embodiments, the serotonin receptor modulator is pruvanserin and the psychedelic is 2C-B, wherein the pruvanserin is administered to pretreat between at least 60 minutes and 240 minutes prior to the administration or release of 2C-B. In some embodiments, the serotonin receptor modulator is pruvanserin and the psychedelic is 2C-B, wherein the pruvanserin is administered to pretreat at least 90 minutes prior to the 2C-B. In some embodiments, the serotonin receptor modulator is pruvanserin and the psychedelic is 2C-B, wherein the pruvanserin is administered to pretreat at least 120 minutes prior to the 2C-B. In some embodiments, the serotonin receptor modulator is pruvanserin and the psychedelic is 2C-B, wherein the pruvanserin is administered to pretreat at least 150 minutes prior to the 2C-B. In some embodiments, the serotonin receptor modulator is pruvanserin and the psychedelic is 2C-B, wherein the pruvanserin is administered to pretreat between about 15 minutes and about 150 minutes prior to the 2C-B. In some embodiments, the serotonin receptor modulator is pruvanserin and the psychedelic is 2C-B, wherein the pruvanserin is administered to pretreat at least 180 minutes prior to the 2C-B. In some embodiments, the serotonin receptor modulator is pruvanserin and the psychedelic is 2C-B, wherein the pruvanserin is administered to pretreat at least 210 minutes prior to the 2C-B.

In some embodiments, the serotonin receptor modulator is pruvanserin and the psychedelic is 2C-B, wherein the pruvanserin is administered to pretreat at least 240 minutes prior to the 2C-B. In some embodiments, the serotonin receptor modulator is pruvanserin and the psychedelic is 2C-B, wherein the pruvanserin is administered to pretreat at least 270 minutes prior to the 2C-B. In some embodiments, the serotonin receptor modulator is pruvanserin and the psychedelic is 2C-B, wherein the pruvanserin is administered to pretreat at least 300 minutes prior to the 2C-B. In some embodiments, the serotonin receptor modulator is pruvanserin and the psychedelic is 2C-B, wherein the pruvanserin is administered to pretreat at least 330 minutes prior to the 2C-B. In some embodiments, the serotonin receptor modulator is pruvanserin and the psychedelic is 2C-B, wherein the pruvanserin is administered to pretreat at least 360 minutes prior to the 2C-B. In some preferred embodiments, the serotonin receptor modulator is pruvanserin and the psychedelic is 2C-B, wherein pruvanserin is administered to pretreat between about 60 minutes and about 180 minutes prior to the administration of 2C-B.

In some embodiments, the serotonin receptor modulator is eplivanserin and the psychedelic is 2C-B HCl, wherein the eplivanserin is administered to pretreat at least 15 minutes prior to the administration of the 2C-B HCl form disclosed herein, including those described in Table 3. In some embodiments, the serotonin receptor modulator is eplivanserin and the psychedelic is 2C-B HCl, wherein the eplivanserin is administered to pretreat between at least 30 minutes prior and 360 minutes prior to the release or administration of the 2C-B HCl form disclosed herein, including those described in Table 3. In some embodiments, the serotonin receptor modulator is eplivanserin and the psychedelic is 2C-B HCl, wherein the eplivanserin is administered to pretreat between at least 60 minutes prior and 360 minutes prior to the release or administration of the 2C-B HCl form disclosed herein, including those described in Table 3. In some embodiments, the serotonin receptor modulator is eplivanserin and the psychedelic is 2C-B HCl, wherein the eplivanserin is administered to pretreat between at least 90 minutes and 240 minutes prior to the 2C-B HCl form disclosed herein, including those described in Table 3. In some embodiments, the serotonin receptor modulator is eplivanserin and the psychedelic is 2C-B HCl, wherein the eplivanserin is administered to pretreat at least 120 minutes prior to the 2C-B HCl form disclosed herein, including those described in Table 3.

In some embodiments, the serotonin receptor modulator is eplivanserin and the psychedelic is 2C-B HCl, wherein the eplivanserin is administered to pretreat at least 150 minutes prior to the 2C-B HCl form disclosed herein, including those described in Table 3. In some embodiments, the serotonin receptor modulator is eplivanserin and the psychedelic is 2C-B HCl, wherein the eplivanserin is administered to pretreat between about 15 minutes and about 150 minutes prior to the 2C-B HCl form disclosed herein, including those described in Table 3. In some embodiments, the serotonin receptor modulator is eplivanserin and the psychedelic is 2C-B HCl, wherein the eplivanserin is administered to pretreat at least 180 minutes prior to the 2C-B HCl form disclosed herein, including those described in Table 3. In some embodiments, the serotonin receptor modulator is eplivanserin and the psychedelic is 2C-B HCl, wherein the eplivanserin is administered to pretreat at least 210 minutes prior to the 2C-B HCl form disclosed herein, including those described in Table 3. In some embodiments, the serotonin receptor modulator is eplivanserin and the psychedelic is 2C-B HCl, wherein the eplivanserin is administered to pretreat at least 240 minutes prior to the 2C-B HCl form disclosed herein, including those described in Table 3.

In some embodiments, the serotonin receptor modulator is eplivanserin and the psychedelic is 2C-B HCl, wherein the eplivanserin is administered to pretreat at least 270 minutes prior to 2C-B HCl. In some embodiments, the serotonin receptor modulator is eplivanserin and the psychedelic is 2C-B HCl, wherein the eplivanserin is administered to pretreat at least 300 minutes prior to the 2C-B HCl form disclosed herein, including those described in Table 3. In some embodiments, the serotonin receptor modulator is eplivanserin and the psychedelic is 2C-B HCl, wherein the eplivanserin is administered to pretreat at least 330 minutes prior to the 2C-B HCl form disclosed herein, including those described in Table 3. In some embodiments, the serotonin receptor modulator is eplivanserin and the psychedelic is 2C-B HCl, wherein the eplivanserin is administered to pretreat at least 360 minutes prior to the 2C-B HCl form disclosed herein, including those described in Table 3.

In some preferred embodiments, the serotonin receptor modulator is eplivanserin and the psychedelic is 2C-B HCl, wherein eplivanserin is administered to pretreat between about 60 minutes and about 180 minutes prior to the administration of the 2C-B HCl form disclosed herein, including those described in Table 3.

In some embodiments, the serotonin receptor modulator is volinanserin and the psychedelic is 2C-B HCl, wherein the volinanserin is administered to pretreat a subject between at least 15 minutes and 360 minutes prior to the administration or release of the 2C-B HCl form disclosed herein, including those described in Table 3. In some embodiments, the serotonin receptor modulator is volinanserin and the psychedelic is 2C-B HCl, wherein the volinanserin is administered to pretreat between at least 30 minutes and 360 minutes prior to the administration or release of the 2C-B HCl form disclosed herein, including those described in Table 3. In some embodiments, the serotonin receptor modulator is volinanserin and the psychedelic is 2C-B HCl, wherein the volinanserin is administered to pretreat between at least 60 minutes and 240 minutes prior to the administration or release of the 2C-B HCl form disclosed herein, including those described in Table 3. In some embodiments, the serotonin receptor modulator is volinanserin and the psychedelic is 2C-B HCl, wherein the volinanserin is administered to pretreat at least 90 minutes prior to the 2C-B HCl form disclosed herein, including those described in Table 3. In some embodiments, the serotonin receptor modulator is volinanserin and the psychedelic is 2C-B HCl, wherein the volinanserin is administered to pretreat at least 120 minutes prior to the 2C-B HCl form disclosed herein, including those described in Table 3.

In some embodiments, the serotonin receptor modulator is volinanserin and the psychedelic is 2C-B HCl, wherein the volinanserin is administered to pretreat at least 150 minutes prior to the 2C-B HCl form disclosed herein, including those described in Table 3. In some embodiments, the serotonin receptor modulator is volinanserin and the psychedelic is 2C-B HCl, wherein the volinanserin is administered to pretreat between about 15 minutes and about 150 minutes prior to the 2C-B HCl form disclosed herein, including those described in Table 3. In some embodiments, the serotonin receptor modulator is volinanserin and the psychedelic is 2C-B HCl, wherein the volinanserin is administered to pretreat at least 180 minutes prior to the 2C-B HCl form disclosed herein, including those described in Table 3. In some embodiments, the serotonin receptor modulator is volinanserin and the psychedelic is 2C-B HCl, wherein the volinanserin is administered to pretreat at least 210 minutes prior to the 2C-B HCl form disclosed herein, including those described in Table 3. In some embodiments, the serotonin receptor modulator is volinanserin and the psychedelic is 2C-B HCl, wherein the volinanserin is administered to pretreat at least 240 minutes prior to the 2C-B HCl form disclosed herein, including those described in Table 3. In some embodiments, the serotonin receptor modulator is volinanserin and the psychedelic is 2C-B HCl, wherein the volinanserin is administered to pretreat at least 270 minutes prior to the 2C-B HCl form disclosed herein, including those described in Table 3. In some embodiments, the serotonin receptor modulator is volinanserin and the psychedelic is 2C-B HCl, wherein the volinanserin is administered to pretreat at least 300 minutes prior to the 2C-B HCl form disclosed herein, including those described in Table 3. In some embodiments, the serotonin receptor modulator is volinanserin and the psychedelic is 2C-B HCl, wherein the volinanserin is administered to pretreat at least 330 minutes prior to the 2C-B HCl form disclosed herein, including those described in Table 3. In some embodiments, the serotonin receptor modulator is volinanserin and the psychedelic is 2C-B HCl, wherein the volinanserin is administered to pretreat at least 360 minutes prior to the 2C-B HCl form disclosed herein, including those described in Table 3. In some preferred embodiments, the serotonin receptor modulator is volinanserin and the psychedelic is 2C-B HCl, wherein volinanserin is administered to pretreat between about 60 minutes and about 180 minutes prior to the administration of the 2C-B HCl form disclosed herein, including those described in Table 3.

In some embodiments, the serotonin receptor modulator is ketanserin and the psychedelic is 2C-B HCl, wherein the ketanserin is administered to pretreat at least 15 minutes prior to the administration of the 2C-B HCl form disclosed herein, including those described in Table 3. In some embodiments, the serotonin receptor modulator is ketanserin and the psychedelic is 2C-B HCl, wherein the ketanserin is administered to pretreat between at least 30 minutes and 360 minutes prior to the administration or release of the 2C-B HCl form disclosed herein, including those described in Table 3. In some embodiments, the serotonin receptor modulator is ketanserin and the psychedelic is 2C-B HCl, wherein the ketanserin is administered to pretreat between at least 60 minutes and 240 minutes prior to the administration or release of the 2C-B HCl form disclosed herein, including those described in Table 3. In some embodiments, the serotonin receptor modulator is ketanserin and the psychedelic is 2C-B HCl, wherein the ketanserin is administered to pretreat at least 90 minutes prior to the 2C-B HCl form disclosed herein, including those described in Table 3. In some embodiments, the serotonin receptor modulator is ketanserin and the psychedelic is 2C-B HCl, wherein the ketanserin is administered to pretreat at least 120 minutes prior to the 2C-B HCl form disclosed herein, including those described in Table 3. In some embodiments, the serotonin receptor modulator is ketanserin and the psychedelic is 2C-B HCl, wherein the ketanserin is administered to pretreat at least 150 minutes prior to the 2C-B HCl form disclosed herein, including those described in Table 3. In some embodiments, the serotonin receptor modulator is ketanserin and the psychedelic is 2C-B HCl, wherein the ketanserin is administered to pretreat between about 15 minutes and about 150 minutes prior to the 2C-B HCl form disclosed herein, including those described in Table 3. In some embodiments, the serotonin receptor modulator is ketanserin and the psychedelic is 2C-B HCl, wherein the ketanserin is administered to pretreat at least 180 minutes prior to the 2C-B HCl form disclosed herein, including those described in Table 3. In some embodiments, the serotonin receptor modulator is ketanserin and the psychedelic is 2C-B HCl, wherein the ketanserin is administered to pretreat at least 210 minutes prior to the 2C-B HCl form disclosed herein, including those described in Table 3. In some embodiments, the serotonin receptor modulator is ketanserin and the psychedelic is 2C-B HCl, wherein the ketanserin is administered to pretreat at least 240 minutes prior to the 2C-B HCl form disclosed herein, including those described in Table 3. In some embodiments, the serotonin receptor modulator is ketanserin and the psychedelic is 2C-B HCl, wherein the ketanserin is administered to pretreat at least 270 minutes prior to the 2C-B HCl form disclosed herein, including those described in Table 3. In some embodiments, the serotonin receptor modulator is ketanserin and the psychedelic is 2C-B HCl, wherein the ketanserin is administered to pretreat at least 300 minutes prior to the 2C-B HCl form disclosed herein, including those described in Table 3. In some embodiments, the serotonin receptor modulator is ketanserin and the psychedelic is 2C-B HCl, wherein the ketanserin is administered to pretreat at least 330 minutes prior to the 2C-B HCl form disclosed herein, including those described in Table 3. In some embodiments, the serotonin receptor modulator is ketanserin and the psychedelic is 2C-B HCl, wherein the ketanserin is administered to pretreat at least 360 minutes prior to the 2C-B HCl form disclosed herein, including those described in Table 3. In some preferred embodiments, the serotonin receptor modulator is ketanserin and the psychedelic is 2C-B HCl, wherein ketanserin is administered to pretreat between about 60 minutes and about 180 minutes prior to the administration of the 2C-B HCl form disclosed herein, including those described in Table 3.

In some embodiments, the serotonin receptor modulator is ritanserin and the psychedelic is 2C-B HCl, wherein the ritanserin is administered to pretreat at least 15 minutes prior to the administration of the 2C-B HCl form disclosed herein, including those described in Table 3. In some embodiments, the serotonin receptor modulator is ritanserin and the psychedelic is 2C-B HCl, wherein the ritanserin is administered to pretreat at least 30 minutes prior to the 2C-B HCl form disclosed herein, including those described in Table 3. In some embodiments, the serotonin receptor modulator is ritanserin and the psychedelic is 2C-B HCl, wherein the ritanserin is administered to pretreat between at least 60 minutes and 240 minutes prior to the administration or release of the 2C-B HCl form disclosed herein, including those described in Table 3. In some embodiments, the serotonin receptor modulator is ritanserin and the psychedelic is 2C-B HCl, wherein the ritanserin is administered to pretreat at least 90 minutes prior to the 2C-B HCl form disclosed herein, including those described in Table 3. In some embodiments, the serotonin receptor modulator is ritanserin and the psychedelic is 2C-B HCl, wherein the ritanserin is administered to pretreat at least 120 minutes prior to the 2C-B HCl form disclosed herein, including those described in Table 3. In some embodiments, the serotonin receptor modulator is ritanserin and the psychedelic is 2C-B HCl, wherein the ritanserin is administered to pretreat at least 150 minutes prior to the 2C-B HCl form disclosed herein, including those described in Table 3. In some embodiments, the serotonin receptor modulator is ritanserin and the psychedelic is 2C-B HCl, wherein the ritanserin is administered to pretreat between about 15 minutes and about 150 minutes prior to the 2C-B HCl form disclosed herein, including those described in Table 3. In some embodiments, the serotonin receptor modulator is ritanserin and the psychedelic is 2C-B HCl, wherein the ritanserin is administered to pretreat at least 180 minutes prior to the 2C-B HCl form disclosed herein, including those described in Table 3. In some embodiments, the serotonin receptor modulator is ritanserin and the psychedelic is 2C-B HCl, wherein the ritanserin is administered to pretreat at least 210 minutes prior to the 2C-B HCl form disclosed herein, including those described in Table 3. In some embodiments, the serotonin receptor modulator is ritanserin and the psychedelic is 2C-B HCl, wherein the ritanserin is administered to pretreat at least 240 minutes prior to the 2C-B HCl form disclosed herein, including those described in Table 3. In some embodiments, the serotonin receptor modulator is ritanserin and the psychedelic is 2C-B HCl, wherein the ritanserin is administered to pretreat at least 270 minutes prior to the 2C-B HCl form disclosed herein, including those described in Table 3. In some embodiments, the serotonin receptor modulator is ritanserin and the psychedelic is 2C-B HCl, wherein the ritanserin is administered to pretreat at least 300 minutes prior to the 2C-B HCl form disclosed herein, including those described in Table 3. In some embodiments, the serotonin receptor modulator is ritanserin and the psychedelic is 2C-B HCl, wherein the ritanserin is administered to pretreat at least 330 minutes prior to the 2C-B HCl form disclosed herein, including those described in Table 3. In some embodiments, the serotonin receptor modulator is ritanserin and the psychedelic is 2C-B HCl, wherein the ritanserin is administered to pretreat at least 360 minutes prior to the 2C-B HCl form disclosed herein, including those described in Table 3. In some preferred embodiments, the serotonin receptor modulator is ritanserin and the psychedelic is 2C-B HCl, wherein ritanserin is administered to pretreat between about 60 minutes and about 180 minutes prior to the administration of the 2C-B HCl form disclosed herein, including those described in Table 3.

In some embodiments, the serotonin receptor modulator is pimavanserin and the psychedelic is 2C-B HCl, wherein the pimavanserin is administered to pretreat at least 15 minutes prior to the administration of the 2C-B HCl form disclosed herein, including those described in Table 3. In some embodiments, the serotonin receptor modulator is pimavanserin and the psychedelic is 2C-B HCl, wherein the pimavanserin is administered to pretreat at least 30 minutes prior to the 2C-B HCl form disclosed herein, including those described in Table 3. In some embodiments, the serotonin receptor modulator is pimavanserin and the psychedelic is 2C-B HCl, wherein the pimavanserin is administered to pretreat between at least 60 minutes and 240 minutes prior to the administration or release of the 2C-B HCl form disclosed herein, including those described in Table 3. In some embodiments, the serotonin receptor modulator is pimavanserin and the psychedelic is 2C-B HCl, wherein the pimavanserin is administered to pretreat at least 90 minutes prior to the 2C-B HCl form disclosed herein, including those described in Table 3. In some embodiments, the serotonin receptor modulator is pimavanserin and the psychedelic is 2C-B HCl, wherein the pimavanserin is administered to pretreat at least 120 minutes prior to the 2C-B HCl form disclosed herein, including those described in Table 3. In some embodiments, the serotonin receptor modulator is pimavanserin and the psychedelic is 2C-B HCl, wherein the pimavanserin is administered to pretreat at least 150 minutes prior to the 2C-B HCl form disclosed herein, including those described in Table 3. In some embodiments, the serotonin receptor modulator is pimavanserin and the psychedelic is 2C-B HCl, wherein the pimavanserin is administered to pretreat between about 15 minutes and about 150 minutes prior to the 2C-B HCl form disclosed herein, including those described in Table 3. In some embodiments, the serotonin receptor modulator is pimavanserin and the psychedelic is 2C-B HCl, wherein the pimavanserin is administered to pretreat at least 180 minutes prior to the 2C-B HCl form disclosed herein, including those described in Table 3. In some embodiments, the serotonin receptor modulator is pimavanserin and the psychedelic is 2C-B HCl, wherein the pimavanserin is administered to pretreat at least 210 minutes prior to the 2C-B HCl form disclosed herein, including those described in Table 3. In some embodiments, the serotonin receptor modulator is pimavanserin and the psychedelic is 2C-B HCl, wherein the pimavanserin is administered to pretreat at least 240 minutes prior to the 2C-B HCl form disclosed herein, including those described in Table 3. In some embodiments, the serotonin receptor modulator is pimavanserin and the psychedelic is 2C-B HCl, wherein the pimavanserin is administered to pretreat at least 270 minutes prior to the 2C-B HCl form disclosed herein, including those described in Table 3. In some embodiments, the serotonin receptor modulator is pimavanserin and the psychedelic is 2C-B HCl, wherein the pimavanserin is administered to pretreat at least 300 minutes prior to the 2C-B HCl form disclosed herein, including those described in Table 3. In some embodiments, the serotonin receptor modulator is pimavanserin and the psychedelic is 2C-B HCl, wherein the pimavanserin is administered to pretreat at least 330 minutes prior to the 2C-B HCl form disclosed herein, including those described in Table 3. In some embodiments, the serotonin receptor modulator is pimavanserin and the psychedelic is 2C-B HCl, wherein the pimavanserin is administered to pretreat at least 360 minutes prior to the 2C-B HCl form disclosed herein, including those described in Table 3. In some preferred embodiments, the serotonin receptor modulator is pimavanserin and the psychedelic is 2C-B HCl, wherein pimavanserin is administered to pretreat between about 60 minutes and about 180 minutes prior to the administration of the 2C-B HCl form disclosed herein, including those described in Table 3. In some embodiments, the serotonin receptor modulator is nelotanserin and the psychedelic is 2C-B HCl, wherein the nelotanserin is administered to pretreat at least 15 minutes prior to the administration of the 2C-B HCl form disclosed herein, including those described in Table 3. In some embodiments, the serotonin receptor modulator is nelotanserin and the psychedelic is 2C-B HCl, wherein the nelotanserin is administered to pretreat at least 30 minutes prior to the 2C-B HCl form disclosed herein, including those described in Table 3. In some embodiments, the serotonin receptor modulator is nelotanserin and the psychedelic is 2C-B HCl, wherein the nelotanserin is administered to pretreat between at least 60 minutes and 240 minutes prior to the administration or release of the 2C-B HCl form disclosed herein, including those described in Table 3. In some embodiments, the serotonin receptor modulator is nelotanserin and the psychedelic is 2C-B HCl, wherein the nelotanserin is administered to pretreat at least 90 minutes prior to the 2C-B HCl form disclosed herein, including those described in Table 3. In some embodiments, the serotonin receptor modulator is nelotanserin and the psychedelic is 2C-B HCl, wherein the nelotanserin is administered to pretreat at least 120 minutes prior to the 2C-B HCl form disclosed herein, including those described in Table 3.

In some embodiments, the serotonin receptor modulator is nelotanserin and the psychedelic is 2C-B HCl, wherein the nelotanserin is administered to pretreat at least 150 minutes prior to the 2C-B HCl form disclosed herein, including those described in Table 3. In some embodiments, the serotonin receptor modulator is nelotanserin and the psychedelic is 2C-B HCl, wherein the nelotanserin is administered to pretreat between about 15 minutes and about 150 minutes prior to the 2C-B HCl form disclosed herein, including those described in Table 3. In some embodiments, the serotonin receptor modulator is nelotanserin and the psychedelic is 2C-B HCl, wherein the nelotanserin is administered to pretreat at least 180 minutes prior to the 2C-B HCl form disclosed herein, including those described in Table 3. In some embodiments, the serotonin receptor modulator is nelotanserin and the psychedelic is 2C-B HCl, wherein the nelotanserin is administered to pretreat at least 210 minutes prior to the 2C-B HCl form disclosed herein, including those described in Table 3. In some embodiments, the serotonin receptor modulator is nelotanserin and the psychedelic is 2C-B HCl, wherein the nelotanserin is administered to pretreat at least 240 minutes prior to the 2C-B HCl form disclosed herein, including those described in Table 3. In some embodiments, the serotonin receptor modulator is nelotanserin and the psychedelic is 2C-B HCl, wherein the nelotanserin is administered to pretreat at least 270 minutes prior to the 2C-B HCl form disclosed herein, including those described in Table 3. In some embodiments, the serotonin receptor modulator is nelotanserin and the psychedelic is 2C-B HCl, wherein the nelotanserin is administered to pretreat at least 300 minutes prior to the 2C-B HCl form disclosed herein, including those described in Table 3. In some embodiments, the serotonin receptor modulator is nelotanserin and the psychedelic is 2C-B HCl, wherein the nelotanserin is administered to pretreat at least 330 minutes prior to the 2C-B HCl form disclosed herein, including those described in Table 3. In some embodiments, the serotonin receptor modulator is nelotanserin and the psychedelic is 2C-B HCl, wherein the nelotanserin is administered to pretreat at least 360 minutes prior to the 2C-B HCl form disclosed herein, including those described in Table 3. In some preferred embodiments, the serotonin receptor modulator is nelotanserin and the psychedelic is 2C-B HCl, wherein nelotanserin is administered to pretreat between about 60 minutes and about 180 minutes prior to the administration of the 2C-B HCl form disclosed herein, including those described in Table 3.

In some embodiments, the serotonin receptor modulator is pruvanserin and the psychedelic is 2C-B HCl, wherein the pruvanserin is administered to pretreat at least 15 minutes prior to the administration of the 2C-B HCl form disclosed herein, including those described in Table 3. In some embodiments, the serotonin receptor modulator is pruvanserin and the psychedelic is 2C-B HCl, wherein the pruvanserin is administered to pretreat at least 30 minutes prior to the 2C-B HCl form disclosed herein, including those described in Table 3. In some embodiments, the serotonin receptor modulator is pruvanserin and the psychedelic is 2C-B HCl, wherein the pruvanserin is administered to pretreat between at least 60 minutes and 240 minutes prior to the administration or release of the 2C-B HCl form disclosed herein, including those described in Table 3. In some embodiments, the serotonin receptor modulator is pruvanserin and the psychedelic is 2C-B HCl, wherein the pruvanserin is administered to pretreat at least 90 minutes prior to the 2C-B HCl form disclosed herein, including those described in Table 3. In some embodiments, the serotonin receptor modulator is pruvanserin and the psychedelic is 2C-B HCl, wherein the pruvanserin is administered to pretreat at least 120 minutes prior to the 2C-B HCl form disclosed herein, including those described in Table 3. In some embodiments, the serotonin receptor modulator is pruvanserin and the psychedelic is 2C-B HCl, wherein the pruvanserin is administered to pretreat at least 150 minutes prior to the 2C-B HCl form disclosed herein, including those described in Table 3. In some embodiments, the serotonin receptor modulator is pruvanserin and the psychedelic is 2C-B HCl, wherein the pruvanserin is administered to pretreat between about 15 minutes and about 150 minutes prior to the 2C-B HCl form disclosed herein, including those described in Table 3. In some embodiments, the serotonin receptor modulator is pruvanserin and the psychedelic is 2C-B HCl, wherein the pruvanserin is administered to pretreat at least 180 minutes prior to the 2C-B HCl form disclosed herein, including those described in Table 3. In some embodiments, the serotonin receptor modulator is pruvanserin and the psychedelic is 2C-B HCl, wherein the pruvanserin is administered to pretreat at least 210 minutes prior to the 2C-B HCl form disclosed herein, including those described in Table 3. In some embodiments, the serotonin receptor modulator is pruvanserin and the psychedelic is 2C-B HCl, wherein the pruvanserin is administered to pretreat at least 240 minutes prior to the 2C-B HCl form disclosed herein, including those described in Table 3. In some embodiments, the serotonin receptor modulator is pruvanserin and the psychedelic is 2C-B HCl, wherein the pruvanserin is administered to pretreat at least 270 minutes prior to the 2C-B HCl form disclosed herein, including those described in Table 3. In some embodiments, the serotonin receptor modulator is pruvanserin and the psychedelic is 2C-B HCl, wherein the pruvanserin is administered to pretreat at least 300 minutes prior to the 2C-B HCl form disclosed herein, including those described in Table 3. In some embodiments, the serotonin receptor modulator is pruvanserin and the psychedelic is 2C-B HCl, wherein the pruvanserin is administered to pretreat at least 330 minutes prior to the 2C-B HCl form disclosed herein, including those described in Table 3. In some embodiments, the serotonin receptor modulator is pruvanserin and the psychedelic is 2C-B HCl, wherein the pruvanserin is administered to pretreat at least 360 minutes prior to the 2C-B HCl form disclosed herein, including those described in Table 3. In some preferred embodiments, the serotonin receptor modulator is pruvanserin and the psychedelic is 2C-B HCl, wherein pruvanserin is administered to pretreat between about 60 minutes and about 180 minutes prior to the administration of the 2C-B HCl form disclosed herein, including those described in Table 3.

In some embodiments, the serotonin receptor modulator is flibanserin and the psychedelic is 2C-B HCl, wherein the flibanserin is administered to pretreat at least 15 minutes prior to the administration of the 2C-B HCl form disclosed herein, including those described in Table 3. In some embodiments, the serotonin receptor modulator is flibanserin and the psychedelic is 2C-B HCl, wherein the flibanserin is administered to pretreat at least 30 minutes prior to the 2C-B HCl form disclosed herein, including those described in Table 3. In some embodiments, the serotonin receptor modulator is flibanserin and the psychedelic is 2C-B HCl, wherein the flibanserin is administered to pretreat between at least 60 minutes and 240 minutes prior to the administration or release of the 2C-B HCl form disclosed herein, including those described in Table 3. In some embodiments, the serotonin receptor modulator is flibanserin and the psychedelic is 2C-B HCl, wherein the flibanserin is administered to pretreat at least 90 minutes prior to the 2C-B HCl form disclosed herein, including those described in Table 3. In some embodiments, the serotonin receptor modulator is flibanserin and the psychedelic is 2C-B HCl, wherein the flibanserin is administered to pretreat at least 120 minutes prior to the 2C-B HCl form disclosed herein, including those described in Table 3. In some embodiments, the serotonin receptor modulator is flibanserin and the psychedelic is 2C-B HCl, wherein the flibanserin is administered to pretreat at least 150 minutes prior to the 2C-B HCl form disclosed herein, including those described in Table 3. In some embodiments, the serotonin receptor modulator is flibanserin and the psychedelic is 2C-B HCl, wherein the flibanserin is administered to pretreat between about 15 minutes and about 150 minutes prior to the 2C-B HCl form disclosed herein, including those described in Table 3. In some embodiments, the serotonin receptor modulator is flibanserin and the psychedelic is 2C-B HCl, wherein the flibanserin is administered to pretreat at least 180 minutes prior to the 2C-B HCl form disclosed herein, including those described in Table 3. In some embodiments, the serotonin receptor modulator is flibanserin and the psychedelic is 2C-B HCl, wherein the flibanserin is administered to pretreat at least 210 minutes prior to the 2C-B HCl form disclosed herein, including those described in Table 3.

In some embodiments, the serotonin receptor modulator is flibanserin and the psychedelic is 2C-B HCl, wherein the flibanserin is administered to pretreat at least 240 minutes prior to the 2C-B HCl form disclosed herein, including those described in Table 3. In some embodiments, the serotonin receptor modulator is flibanserin and the psychedelic is 2C-B HCl, wherein the flibanserin is administered to pretreat at least 270 minutes prior to the 2C-B HCl form disclosed herein, including those described in Table 3. In some embodiments, the serotonin receptor modulator is flibanserin and the psychedelic is 2C-B HCl, wherein the flibanserin is administered to pretreat at least 300 minutes prior to the 2C-B HCl form disclosed herein, including those described in Table 3. In some embodiments, the serotonin receptor modulator is flibanserin and the psychedelic is 2C-B HCl, wherein the flibanserin is administered to pretreat at least 330 minutes prior to the 2C-B HCl form disclosed herein, including those described in Table 3. In some embodiments, the serotonin receptor modulator is flibanserin and the psychedelic is 2C-B HCl, wherein the flibanserin is administered to pretreat at least 360 minutes prior to the 2C-B HCl form disclosed herein, including those described in Table 3. In some preferred embodiments, the serotonin receptor modulator is flibanserin and the psychedelic is 2C-B HCl, wherein flibanserin is administered to pretreat between about 60 minutes and about 180 minutes prior to the administration of the 2C-B HCl form disclosed herein, including those described in Table 3.

In some embodiments, the serotonin receptor modulator is olanzapine and the psychedelic is 2C-B HCl, wherein the olanzapine is administered to pretreat at least 15 minutes prior to the administration of the 2C-B HCl form disclosed herein, including those described in Table 3. In some embodiments, the serotonin receptor modulator is olanzapine and the psychedelic is 2C-B HCl, wherein the olanzapine is administered to pretreat at least 30 minutes prior to the 2C-B HCl form disclosed herein, including those described in Table 3. In some embodiments, the serotonin receptor modulator is olanzapine and the psychedelic is 2C-B HCl, wherein the olanzapine is administered to pretreat between at least 60 minutes and 240 minutes prior to the administration or release of the 2C-B HCl form disclosed herein, including those described in Table 3. In some embodiments, the serotonin receptor modulator is olanzapine and the psychedelic is 2C-B HCl, wherein the olanzapine is administered to pretreat at least 90 minutes prior to the 2C-B HCl form disclosed herein, including those described in Table 3. In some embodiments, the serotonin receptor modulator is olanzapine and the psychedelic is 2C-B HCl, wherein the olanzapine is administered to pretreat at least 120 minutes prior to the 2C-B HCl form disclosed herein, including those described in Table 3. In some embodiments, the serotonin receptor modulator is olanzapine and the psychedelic is 2C-B HCl, wherein the olanzapine is administered to pretreat at least 150 minutes prior to the 2C-B HCl form disclosed herein, including those described in Table 3. In some embodiments, the serotonin receptor modulator is olanzapine and the psychedelic is 2C-B HCl, wherein the olanzapine is administered to pretreat between about 15 minutes and about 150 minutes prior to the 2C-B HCl form disclosed herein, including those described in Table 3. In some embodiments, the serotonin receptor modulator is olanzapine and the psychedelic is 2C-B HCl, wherein the olanzapine is administered to pretreat at least 180 minutes prior to the 2C-B HCl form disclosed herein, including those described in Table 3. In some embodiments, the serotonin receptor modulator is olanzapine and the psychedelic is 2C-B HCl, wherein the olanzapine is administered to pretreat at least 210 minutes prior to the 2C-B HCl form disclosed herein, including those described in Table 3.

In some embodiments, the serotonin receptor modulator is olanzapine and the psychedelic is 2C-B HCl, wherein the olanzapine is administered to pretreat at least 240 minutes prior to the 2C-B HCl form disclosed herein, including those described in Table 3. In some embodiments, the serotonin receptor modulator is olanzapine and the psychedelic is 2C-B HCl, wherein the olanzapine is administered to pretreat at least 270 minutes prior to the 2C-B HCl form disclosed herein, including those described in Table 3. In some embodiments, the serotonin receptor modulator is olanzapine and the psychedelic is 2C-B HCl, wherein the olanzapine is administered to pretreat at least 300 minutes prior to the 2C-B HCl form disclosed herein, including those described in Table 3. In some embodiments, the serotonin receptor modulator is olanzapine and the psychedelic is 2C-B HCl, wherein the olanzapine is administered to pretreat at least 330 minutes prior to the 2C-B HCl form disclosed herein, including those described in Table 3. In some embodiments, the serotonin receptor modulator is olanzapine and the psychedelic is 2C-B HCl, wherein the olanzapine is administered to pretreat at least 360 minutes prior to the 2C-B HCl form disclosed herein, including those described in Table 3. In some preferred embodiments, the serotonin receptor modulator is olanzapine and the psychedelic is 2C-B HCl, wherein olanzapine is administered to pretreat between about 60 minutes and about 180 minutes prior to the administration of the 2C-B HCl form disclosed herein, including those described in Table 3.

In some embodiments, the serotonin receptor modulator is risperidone and the psychedelic is 2C-B HCl, wherein the risperidone is administered to pretreat at least 15 minutes prior to the administration of the 2C-B HCl form disclosed herein, including those described in Table 3. In some embodiments, the serotonin receptor modulator is risperidone and the psychedelic is 2C-B HCl, wherein the risperidone is administered to pretreat at least 30 minutes prior to the 2C-B HCl form disclosed herein, including those described in Table 3. In some embodiments, the serotonin receptor modulator is risperidone and the psychedelic is 2C-B HCl, wherein the risperidone is administered to pretreat between at least 60 minutes and 240 minutes prior to the administration or release of the 2C-B HCl form disclosed herein, including those described in Table 3. In some embodiments, the serotonin receptor modulator is risperidone and the psychedelic is 2C-B HCl, wherein the risperidone is administered to pretreat at least 90 minutes prior to the 2C-B HCl form disclosed herein, including those described in Table 3. In some embodiments, the serotonin receptor modulator is risperidone and the psychedelic is 2C-B HCl, wherein the risperidone is administered to pretreat at least 120 minutes prior to the 2C-B HCl form disclosed herein, including those described in Table 3. In some embodiments, the serotonin receptor modulator is risperidone and the psychedelic is 2C-B HCl, wherein the risperidone is administered to pretreat at least 150 minutes prior to the 2C-B HCl form disclosed herein, including those described in Table 3. In some embodiments, the serotonin receptor modulator is risperidone and the psychedelic is 2C-B HCl, wherein the risperidone is administered to pretreat between about 15 minutes and about 150 minutes prior to the 2C-B HCl form disclosed herein, including those described in Table 3. In some embodiments, the serotonin receptor modulator is risperidone and the psychedelic is 2C-B HCl, wherein the risperidone is administered to pretreat at least 180 minutes prior to the 2C-B HCl form disclosed herein, including those described in Table 3. In some embodiments, the serotonin receptor modulator is risperidone and the psychedelic is 2C-B HCl, wherein the risperidone is administered to pretreat at least 210 minutes prior to the 2C-B HCl form disclosed herein, including those described in Table 3.

In some embodiments, the serotonin receptor modulator is risperidone and the psychedelic is 2C-B HCl, wherein the risperidone is administered to pretreat at least 240 minutes prior to the 2C-B HCl form disclosed herein, including those described in Table 3. In some embodiments, the serotonin receptor modulator is risperidone and the psychedelic is 2C-B HCl, wherein the risperidone is administered to pretreat at least 270 minutes prior to the 2C-B HCl form disclosed herein, including those described in Table 3. In some embodiments, the serotonin receptor modulator is risperidone and the psychedelic is 2C-B HCl, wherein the risperidone is administered to pretreat at least 300 minutes prior to the 2C-B HCl form disclosed herein, including those described in Table 3. In some embodiments, the serotonin receptor modulator is risperidone and the psychedelic is 2C-B HCl, wherein the risperidone is administered to pretreat at least 330 minutes prior to the 2C-B HCl form disclosed herein, including those described in Table 3. In some embodiments, the serotonin receptor modulator is risperidone and the psychedelic is 2C-B HCl, wherein the risperidone is administered to pretreat at least 360 minutes prior to the 2C-B HCl form disclosed herein, including those described in Table 3. In some preferred embodiments, the serotonin receptor modulator is risperidone and the psychedelic is 2C-B HCl, wherein risperidone is administered to pretreat between about 60 minutes and about 180 minutes prior to the administration of the 2C-B HCl form disclosed herein, including those described in Table 3.

In some embodiments, the serotonin receptor modulator is quetiapine and the psychedelic is 2C-B HCl, wherein the quetiapine is administered to pretreat at least 15 minutes prior to the administration of the 2C-B HCl form disclosed herein, including those described in Table 3. In some embodiments, the serotonin receptor modulator is quetiapine and the psychedelic is 2C-B HCl, wherein the quetiapine is administered to pretreat at least 30 minutes prior to the 2C-B HCl form disclosed herein, including those described in Table 3. In some embodiments, the serotonin receptor modulator is quetiapine and the psychedelic is 2C-B HCl, wherein the quetiapine is administered to pretreat between at least 60 minutes and 240 minutes prior to the administration or release of the 2C-B HCl form disclosed herein, including those described in Table 3. In some embodiments, the serotonin receptor modulator is quetiapine and the psychedelic is 2C-B HCl, wherein the quetiapine is administered to pretreat at least 90 minutes prior to the 2C-B HCl form disclosed herein, including those described in Table 3. In some embodiments, the serotonin receptor modulator is quetiapine and the psychedelic is 2C-B HCl, wherein the quetiapine is administered to pretreat at least 120 minutes prior to the 2C-B HCl form disclosed herein, including those described in Table 3. In some embodiments, the serotonin receptor modulator is quetiapine and the psychedelic is 2C-B HCl, wherein the quetiapine is administered to pretreat at least 150 minutes prior to the 2C-B HCl form disclosed herein, including those described in Table 3. In some embodiments, the serotonin receptor modulator is quetiapine and the psychedelic is 2C-B HCl, wherein the quetiapine is administered to pretreat between about 15 minutes and about 150 minutes prior to the 2C-B HCl form disclosed herein, including those described in Table 3. In some embodiments, the serotonin receptor modulator is quetiapine and the psychedelic is 2C-B HCl, wherein the quetiapine is administered to pretreat at least 180 minutes prior to the 2C-B HCl form disclosed herein, including those described in Table 3. In some embodiments, the serotonin receptor modulator is quetiapine and the psychedelic is 2C-B HCl, wherein the quetiapine is administered to pretreat at least 210 minutes prior to the 2C-B HCl form disclosed herein, including those described in Table 3.

In some embodiments, the serotonin receptor modulator is quetiapine and the psychedelic is 2C-B HCl, wherein the quetiapine is administered to pretreat at least 240 minutes prior to the 2C-B HCl form disclosed herein, including those described in Table 3. In some embodiments, the serotonin receptor modulator is quetiapine and the psychedelic is 2C-B HCl, wherein the quetiapine is administered to pretreat at least 270 minutes prior to the 2C-B HCl form disclosed herein, including those described in Table 3. In some embodiments, the serotonin receptor modulator is quetiapine and the psychedelic is 2C-B HCl, wherein the quetiapine is administered to pretreat at least 300 minutes prior to the 2C-B HCl form disclosed herein, including those described in Table 3. In some embodiments, the serotonin receptor modulator is quetiapine and the psychedelic is 2C-B HCl, wherein the quetiapine is administered to pretreat at least 330 minutes prior to the 2C-B HCl form disclosed herein, including those described in Table 3. In some embodiments, the serotonin receptor modulator is quetiapine and the psychedelic is 2C-B HCl, wherein the quetiapine is administered to pretreat at least 360 minutes prior to the 2C-B HCl form disclosed herein, including those described in Table 3. In some preferred embodiments, the serotonin receptor modulator is quetiapine and the psychedelic is 2C-B HCl, wherein quetiapine is administered to pretreat between about 60 minutes and about 180 minutes prior to the administration of the 2C-B HCl form disclosed herein, including those described in Table 3.

In some embodiments, the serotonin receptor modulator is eplivanserin and the psychedelic is 2C-B, wherein the eplivanserin is administered to pretreat at least 15 minutes prior to the administration of the 2C-B form disclosed herein including those described in Table 18. In some embodiments, the serotonin receptor modulator is eplivanserin and the psychedelic is 2C-B, wherein the eplivanserin is administered to pretreat between at least 30 minutes prior and 360 minutes prior to the release or administration of the 2C-B form disclosed herein including those described in Table 18. In some embodiments, the serotonin receptor modulator is eplivanserin and the psychedelic is 2C-B, wherein the eplivanserin is administered to pretreat between at least 60 minutes prior and 360 minutes prior to the release or administration of the 2C-B form disclosed herein including those described in Table 18. In some embodiments, the serotonin receptor modulator is eplivanserin and the psychedelic is 2C-B, wherein the eplivanserin is administered to pretreat at between least 90 minutes and 240 minutes prior to the 2C-B form disclosed herein including those described in Table 18. In some embodiments, the serotonin receptor modulator is eplivanserin and the psychedelic is 2C-B, wherein the eplivanserin is administered to pretreat at least 120 minutes prior to the 2C-B form disclosed herein including those described in Table 18.

In some embodiments, the serotonin receptor modulator is eplivanserin and the psychedelic is 2C-B, wherein the eplivanserin is administered to pretreat at least 150 minutes prior to the 2C-B form disclosed herein including those described in Table 18. In some embodiments, the serotonin receptor modulator is eplivanserin and the psychedelic is 2C-B, wherein the eplivanserin is administered to pretreat between about 15 minutes and about 150 minutes prior to the 2C-B form disclosed herein including those described in Table 18. In some embodiments, the serotonin receptor modulator is eplivanserin and the psychedelic is 2C-B, wherein the eplivanserin is administered to pretreat at least 180 minutes prior to the 2C-B form disclosed herein including those described in Table 18. In some embodiments, the serotonin receptor modulator is eplivanserin and the psychedelic is 2C-B, wherein the eplivanserin is administered to pretreat at least 210 minutes prior to the 2C-B form disclosed herein including those described in Table 18. In some embodiments, the serotonin receptor modulator is eplivanserin and the psychedelic is 2C-B, wherein the eplivanserin is administered to pretreat at least 240 minutes prior to the 2C-B form disclosed herein including those described in Table 18.

In some embodiments, the serotonin receptor modulator is eplivanserin and the psychedelic is 2C-B, wherein the eplivanserin is administered to pretreat at least 270 minutes prior to 2C-B. In some embodiments, the serotonin receptor modulator is eplivanserin and the psychedelic is 2C-B, wherein the eplivanserin is administered to pretreat at least 300 minutes prior to the 2C-B form disclosed herein including those described in Table 18. In some embodiments, the serotonin receptor modulator is eplivanserin and the psychedelic is 2C-B, wherein the eplivanserin is administered to pretreat at least 330 minutes prior to the 2C-B form disclosed herein including those described in Table 18. In some embodiments, the serotonin receptor modulator is eplivanserin and the psychedelic is 2C-B, wherein the eplivanserin is administered to pretreat at least 360 minutes prior to the 2C-B form disclosed herein including those described in Table 18.

In some preferred embodiments, the serotonin receptor modulator is eplivanserin and the psychedelic is 2C-B, wherein eplivanserin is administered to pretreat between about 60 minutes and about 180 minutes prior to the administration of the 2C-B form disclosed herein including those described in Table 18.

In some embodiments, the serotonin receptor modulator is volinanserin and the psychedelic is 2C-B, wherein the volinanserin is administered to pretreat a subject between at least 15 minutes and 360 minutes prior to the administration or release of the 2C-B form disclosed herein including those described in Table 18. In some embodiments, the serotonin receptor modulator is volinanserin and the psychedelic is 2C-B, wherein the volinanserin is administered to pretreat between at least 30 minutes and 360 minutes prior to the administration or release of the 2C-B form disclosed herein including those described in Table 18. In some embodiments, the serotonin receptor modulator is volinanserin and the psychedelic is 2C-B, wherein the volinanserin is administered to pretreat between at least 60 minutes and 240 minutes prior to the administration or release of the 2C-B form disclosed herein including those described in Table 18. In some embodiments, the serotonin receptor modulator is volinanserin and the psychedelic is 2C-B, wherein the volinanserin is administered to pretreat at least 90 minutes prior to the 2C-B form disclosed herein including those described in Table 18. In some embodiments, the serotonin receptor modulator is volinanserin and the psychedelic is 2C-B, wherein the volinanserin is administered to pretreat at least 120 minutes prior to the 2C-B form disclosed herein including those described in Table 18.

In some embodiments, the serotonin receptor modulator is volinanserin and the psychedelic is 2C-B, wherein the volinanserin is administered to pretreat at least 150 minutes prior to the 2C-B form disclosed herein including those described in Table 18. In some embodiments, the serotonin receptor modulator is volinanserin and the psychedelic is 2C-B, wherein the volinanserin is administered to pretreat between about 15 minutes and about 150 minutes prior to the 2C-B form disclosed herein including those described in Table 18. In some embodiments, the serotonin receptor modulator is volinanserin and the psychedelic is 2C-B, wherein the volinanserin is administered to pretreat at least 180 minutes prior to the 2C-B form disclosed herein including those described in Table 18. In some embodiments, the serotonin receptor modulator is volinanserin and the psychedelic is 2C-B, wherein the volinanserin is administered to pretreat at least 210 minutes prior to the 2C-B form disclosed herein including those described in Table 18. In some embodiments, the serotonin receptor modulator is volinanserin and the psychedelic is 2C-B, wherein the volinanserin is administered to pretreat at least 240 minutes prior to the 2C-B form disclosed herein including those described in Table 18. In some embodiments, the serotonin receptor modulator is volinanserin and the psychedelic is 2C-B, wherein the volinanserin is administered to pretreat at least 270 minutes prior to the 2C-B form disclosed herein including those described in Table 18. In some embodiments, the serotonin receptor modulator is volinanserin and the psychedelic is 2C-B, wherein the volinanserin is administered to pretreat at least 300 minutes prior to the 2C-B form disclosed herein including those described in Table 18. In some embodiments, the serotonin receptor modulator is volinanserin and the psychedelic is 2C-B, wherein the volinanserin is administered to pretreat at least 330 minutes prior to the 2C-B form disclosed herein including those described in Table 18. In some embodiments, the serotonin receptor modulator is volinanserin and the psychedelic is 2C-B, wherein the volinanserin is administered to pretreat at least 360 minutes prior to the 2C-B form disclosed herein including those described in Table 18. In some preferred embodiments, the serotonin receptor modulator is volinanserin and the psychedelic is 2C-B, wherein volinanserin is administered to pretreat between about 60 minutes and about 180 minutes prior to the administration of the 2C-B form disclosed herein including those described in Table 18.

In some embodiments, the serotonin receptor modulator is ketanserin and the psychedelic is 2C-B, wherein the ketanserin is administered to pretreat at least 15 minutes prior to the administration of the 2C-B form disclosed herein including those described in Table 18. In some embodiments, the serotonin receptor modulator is ketanserin and the psychedelic is 2C-B, wherein the ketanserin is administered to pretreat between at least 30 minutes and 360 minutes prior to the administration or release of the 2C-B form disclosed herein including those described in Table 18. In some embodiments, the serotonin receptor modulator is ketanserin and the psychedelic is 2C-B, wherein the ketanserin is administered to pretreat between at least 60 minutes and 240 minutes prior to the administration or release of the 2C-B form disclosed herein including those described in Table 18. In some embodiments, the serotonin receptor modulator is ketanserin and the psychedelic is 2C-B, wherein the ketanserin is administered to pretreat at least 90 minutes prior to the 2C-B form disclosed herein including those described in Table 18. In some embodiments, the serotonin receptor modulator is ketanserin and the psychedelic is 2C-B, wherein the ketanserin is administered to pretreat at least 120 minutes prior to the 2C-B form disclosed herein including those described in Table 18. In some embodiments, the serotonin receptor modulator is ketanserin and the psychedelic is 2C-B, wherein the ketanserin is administered to pretreat at least 150 minutes prior to the 2C-B form disclosed herein including those described in Table 18. In some embodiments, the serotonin receptor modulator is ketanserin and the psychedelic is 2C-B, wherein the ketanserin is administered to pretreat between about 15 minutes and about 150 minutes prior to the 2C-B form disclosed herein including those described in Table 18. In some embodiments, the serotonin receptor modulator is ketanserin and the psychedelic is 2C-B, wherein the ketanserin is administered to pretreat at least 180 minutes prior to the 2C-B form disclosed herein including those described in Table 18. In some embodiments, the serotonin receptor modulator is ketanserin and the psychedelic is 2C-B, wherein the ketanserin is administered to pretreat at least 210 minutes prior to the 2C-B form disclosed herein including those described in Table 18. In some embodiments, the serotonin receptor modulator is ketanserin and the psychedelic is 2C-B, wherein the ketanserin is administered to pretreat at least 240 minutes prior to the 2C-B form disclosed herein including those described in Table 18. In some embodiments, the serotonin receptor modulator is ketanserin and the psychedelic is 2C-B, wherein the ketanserin is administered to pretreat at least 270 minutes prior to the 2C-B form disclosed herein including those described in Table 18. In some embodiments, the serotonin receptor modulator is ketanserin and the psychedelic is 2C-B, wherein the ketanserin is administered to pretreat at least 300 minutes prior to the 2C-B form disclosed herein including those described in Table 18. In some embodiments, the serotonin receptor modulator is ketanserin and the psychedelic is 2C-B, wherein the ketanserin is administered to pretreat at least 330 minutes prior to the 2C-B form disclosed herein including those described in Table 18. In some embodiments, the serotonin receptor modulator is ketanserin and the psychedelic is 2C-B, wherein the ketanserin is administered to pretreat at least 360 minutes prior to the 2C-B form disclosed herein including those described in Table 18. In some preferred embodiments, the serotonin receptor modulator is ketanserin and the psychedelic is 2C-B, wherein ketanserin is administered to pretreat between about 60 minutes and about 180 minutes prior to the administration of the 2C-B form disclosed herein including those described in Table 18.

In some embodiments, the serotonin receptor modulator is ritanserin and the psychedelic is 2C-B, wherein the ritanserin is administered to pretreat at least 15 minutes prior to the administration of the 2C-B form disclosed herein including those described in Table 18. In some embodiments, the serotonin receptor modulator is ritanserin and the psychedelic is 2C-B, wherein the ritanserin is administered to pretreat at least 30 minutes prior to the 2C-B form disclosed herein including those described in Table 18. In some embodiments, the serotonin receptor modulator is ritanserin and the psychedelic is 2C-B, wherein the ritanserin is administered to pretreat between at least 60 minutes and 240 minutes prior to the administration or release of the 2C-B form disclosed herein including those described in Table 18. In some embodiments, the serotonin receptor modulator is ritanserin and the psychedelic is 2C-B, wherein the ritanserin is administered to pretreat at least 90 minutes prior to the 2C-B form disclosed herein including those described in Table 18. In some embodiments, the serotonin receptor modulator is ritanserin and the psychedelic is 2C-B, wherein the ritanserin is administered to pretreat at least 120 minutes prior to the 2C-B form disclosed herein including those described in Table 18. In some embodiments, the serotonin receptor modulator is ritanserin and the psychedelic is 2C-B, wherein the ritanserin is administered to pretreat at least 150 minutes prior to the 2C-B form disclosed herein including those described in Table 18. In some embodiments, the serotonin receptor modulator is ritanserin and the psychedelic is 2C-B, wherein the ritanserin is administered to pretreat between about 15 minutes and about 150 minutes prior to the 2C-B form disclosed herein including those described in Table 18. In some embodiments, the serotonin receptor modulator is ritanserin and the psychedelic is 2C-B, wherein the ritanserin is administered to pretreat at least 180 minutes prior to the 2C-B form disclosed herein including those described in Table 18. In some embodiments, the serotonin receptor modulator is ritanserin and the psychedelic is 2C-B, wherein the ritanserin is administered to pretreat at least 210 minutes prior to the 2C-B form disclosed herein including those described in Table 18. In some embodiments, the serotonin receptor modulator is ritanserin and the psychedelic is 2C-B, wherein the ritanserin is administered to pretreat at least 240 minutes prior to the 2C-B form disclosed herein including those described in Table 18. In some embodiments, the serotonin receptor modulator is ritanserin and the psychedelic is 2C-B, wherein the ritanserin is administered to pretreat at least 270 minutes prior to the 2C-B form disclosed herein including those described in Table 18. In some embodiments, the serotonin receptor modulator is ritanserin and the psychedelic is 2C-B, wherein the ritanserin is administered to pretreat at least 300 minutes prior to the 2C-B form disclosed herein including those described in Table 18. In some embodiments, the serotonin receptor modulator is ritanserin and the psychedelic is 2C-B, wherein the ritanserin is administered to pretreat at least 330 minutes prior to the 2C-B form disclosed herein including those described in Table 18. In some embodiments, the serotonin receptor modulator is ritanserin and the psychedelic is 2C-B, wherein the ritanserin is administered to pretreat at least 360 minutes prior to the 2C-B form disclosed herein including those described in Table 18. In some preferred embodiments, the serotonin receptor modulator is ritanserin and the psychedelic is 2C-B, wherein ritanserin is administered to pretreat between about 60 minutes and about 180 minutes prior to the administration of the 2C-B form disclosed herein including those described in Table 18.

In some embodiments, the serotonin receptor modulator is pimavanserin and the psychedelic is 2C-B, wherein the pimavanserin is administered to pretreat at least 15 minutes prior to the administration of the 2C-B form disclosed herein including those described in Table 18. In some embodiments, the serotonin receptor modulator is pimavanserin and the psychedelic is 2C-B, wherein the pimavanserin is administered to pretreat at least 30 minutes prior to the 2C-B form disclosed herein including those described in Table 18. In some embodiments, the serotonin receptor modulator is pimavanserin and the psychedelic is 2C-B, wherein the pimavanserin is administered to pretreat between at least 60 minutes and 240 minutes prior to the administration or release of the 2C-B form disclosed herein including those described in Table 18. In some embodiments, the serotonin receptor modulator is pimavanserin and the psychedelic is 2C-B, wherein the pimavanserin is administered to pretreat at least 90 minutes prior to the 2C-B form disclosed herein including those described in Table 18. In some embodiments, the serotonin receptor modulator is pimavanserin and the psychedelic is 2C-B, wherein the pimavanserin is administered to pretreat at least 120 minutes prior to the 2C-B form disclosed herein including those described in Table 18. In some embodiments, the serotonin receptor modulator is pimavanserin and the psychedelic is 2C-B, wherein the pimavanserin is administered to pretreat at least 150 minutes prior to the 2C-B form disclosed herein including those described in Table 18. In some embodiments, the serotonin receptor modulator is pimavanserin and the psychedelic is 2C-B, wherein the pimavanserin is administered to pretreat between about 15 minutes and about 150 minutes prior to the 2C-B form disclosed herein including those described in Table 18. In some embodiments, the serotonin receptor modulator is pimavanserin and the psychedelic is 2C-B, wherein the pimavanserin is administered to pretreat at least 180 minutes prior to the 2C-B form disclosed herein including those described in Table 18. In some embodiments, the serotonin receptor modulator is pimavanserin and the psychedelic is 2C-B, wherein the pimavanserin is administered to pretreat at least 210 minutes prior to the 2C-B form disclosed herein including those described in Table 18. In some embodiments, the serotonin receptor modulator is pimavanserin and the psychedelic is 2C-B, wherein the pimavanserin is administered to pretreat at least 240 minutes prior to the 2C-B form disclosed herein including those described in Table 18. In some embodiments, the serotonin receptor modulator is pimavanserin and the psychedelic is 2C-B, wherein the pimavanserin is administered to pretreat at least 270 minutes prior to the 2C-B form disclosed herein including those described in Table 18. In some embodiments, the serotonin receptor modulator is pimavanserin and the psychedelic is 2C-B, wherein the pimavanserin is administered to pretreat at least 300 minutes prior to the 2C-B form disclosed herein including those described in Table 18. In some embodiments, the serotonin receptor modulator is pimavanserin and the psychedelic is 2C-B, wherein the pimavanserin is administered to pretreat at least 330 minutes prior to the 2C-B form disclosed herein including those described in Table 18. In some embodiments, the serotonin receptor modulator is pimavanserin and the psychedelic is 2C-B, wherein the pimavanserin is administered to pretreat at least 360 minutes prior to the 2C-B form disclosed herein including those described in Table 18. In some preferred embodiments, the serotonin receptor modulator is pimavanserin and the psychedelic is 2C-B, wherein pimavanserin is administered to pretreat between about 60 minutes and about 180 minutes prior to the administration of the 2C-B form disclosed herein including those described in Table 18.

In some embodiments, the serotonin receptor modulator is nelotanserin and the psychedelic is 2C-B, wherein the nelotanserin is administered to pretreat at least 15 minutes prior to the administration of the 2C-B form disclosed herein including those described in Table 18. In some embodiments, the serotonin receptor modulator is nelotanserin and the psychedelic is 2C-B, wherein the nelotanserin is administered to pretreat at least 30 minutes prior to the 2C-B form disclosed herein including those described in Table 18. In some embodiments, the serotonin receptor modulator is nelotanserin and the psychedelic is 2C-B, wherein the nelotanserin is administered to pretreat between at least 60 minutes and 240 minutes prior to the administration or release of the 2C-B form disclosed herein including those described in Table 18. In some embodiments, the serotonin receptor modulator is nelotanserin and the psychedelic is 2C-B, wherein the nelotanserin is administered to pretreat at least 90 minutes prior to the 2C-B form disclosed herein including those described in Table 18. In some embodiments, the serotonin receptor modulator is nelotanserin and the psychedelic is 2C-B, wherein the nelotanserin is administered to pretreat at least 120 minutes prior to the 2C-B form disclosed herein including those described in Table 18.

In some embodiments, the serotonin receptor modulator is nelotanserin and the psychedelic is 2C-B, wherein the nelotanserin is administered to pretreat at least 150 minutes prior to the 2C-B form disclosed herein including those described in Table 18. In some embodiments, the serotonin receptor modulator is nelotanserin and the psychedelic is 2C-B, wherein the nelotanserin is administered to pretreat between about 15 minutes and about 150 minutes prior to the 2C-B form disclosed herein including those described in Table 18. In some embodiments, the serotonin receptor modulator is nelotanserin and the psychedelic is 2C-B, wherein the nelotanserin is administered to pretreat at least 180 minutes prior to the 2C-B form disclosed herein including those described in Table 18. In some embodiments, the serotonin receptor modulator is nelotanserin and the psychedelic is 2C-B, wherein the nelotanserin is administered to pretreat at least 210 minutes prior to the 2C-B form disclosed herein including those described in Table 18. In some embodiments, the serotonin receptor modulator is nelotanserin and the psychedelic is 2C-B, wherein the nelotanserin is administered to pretreat at least 240 minutes prior to the 2C-B form disclosed herein including those described in Table 18. In some embodiments, the serotonin receptor modulator is nelotanserin and the psychedelic is 2C-B, wherein the nelotanserin is administered to pretreat at least 270 minutes prior to the 2C-B form disclosed herein including those described in Table 18.

In some embodiments, the serotonin receptor modulator is nelotanserin and the psychedelic is 2C-B, wherein the nelotanserin is administered to pretreat at least 300 minutes prior to the 2C-B form disclosed herein including those described in Table 18. In some embodiments, the serotonin receptor modulator is nelotanserin and the psychedelic is 2C-B, wherein the nelotanserin is administered to pretreat at least 330 minutes prior to the 2C-B form disclosed herein including those described in Table 18. In some embodiments, the serotonin receptor modulator is nelotanserin and the psychedelic is 2C-B, wherein the nelotanserin is administered to pretreat at least 360 minutes prior to the 2C-B form disclosed herein including those described in Table 18. In some preferred embodiments, the serotonin receptor modulator is nelotanserin and the psychedelic is 2C-B, wherein nelotanserin is administered to pretreat between about 60 minutes and about 180 minutes prior to the administration of the 2C-B form disclosed herein including those described in Table 18.

In some embodiments, the serotonin receptor modulator is pruvanserin and the psychedelic is 2C-B, wherein the pruvanserin is administered to pretreat at least 15 minutes prior to the administration of the 2C-B form disclosed herein including those described in Table 18. In some embodiments, the serotonin receptor modulator is pruvanserin and the psychedelic is 2C-B, wherein the pruvanserin is administered to pretreat at least 30 minutes prior to the 2C-B form disclosed herein including those described in Table 18. In some embodiments, the serotonin receptor modulator is pruvanserin and the psychedelic is 2C-B, wherein the pruvanserin is administered to pretreat between at least 60 minutes and 240 minutes prior to the administration or release of the 2C-B form disclosed herein including those described in Table 18. In some embodiments, the serotonin receptor modulator is pruvanserin and the psychedelic is 2C-B, wherein the pruvanserin is administered to pretreat at least 90 minutes prior to the 2C-B form disclosed herein including those described in Table 18. In some embodiments, the serotonin receptor modulator is pruvanserin and the psychedelic is 2C-B, wherein the pruvanserin is administered to pretreat at least 120 minutes prior to the 2C-B form disclosed herein including those described in Table 18. In some embodiments, the serotonin receptor modulator is pruvanserin and the psychedelic is 2C-B, wherein the pruvanserin is administered to pretreat at least 150 minutes prior to the 2C-B form disclosed herein including those described in Table 18. In some embodiments, the serotonin receptor modulator is pruvanserin and the psychedelic is 2C-B, wherein the pruvanserin is administered to pretreat between about 15 minutes and about 150 minutes prior to the 2C-B form disclosed herein including those described in Table 18. In some embodiments, the serotonin receptor modulator is pruvanserin and the psychedelic is 2C-B, wherein the pruvanserin is administered to pretreat at least 180 minutes prior to the 2C-B form disclosed herein including those described in Table 18. In some embodiments, the serotonin receptor modulator is pruvanserin and the psychedelic is 2C-B, wherein the pruvanserin is administered to pretreat at least 210 minutes prior to the 2C-B form disclosed herein including those described in Table 18.

In some embodiments, the serotonin receptor modulator is pruvanserin and the psychedelic is 2C-B, wherein the pruvanserin is administered to pretreat at least 240 minutes prior to the 2C-B form disclosed herein including those described in Table 18. In some embodiments, the serotonin receptor modulator is pruvanserin and the psychedelic is 2C-B, wherein the pruvanserin is administered to pretreat at least 270 minutes prior to the 2C-B form disclosed herein including those described in Table 18. In some embodiments, the serotonin receptor modulator is pruvanserin and the psychedelic is 2C-B, wherein the pruvanserin is administered to pretreat at least 300 minutes prior to the 2C-B form disclosed herein including those described in Table 18. In some embodiments, the serotonin receptor modulator is pruvanserin and the psychedelic is 2C-B, wherein the pruvanserin is administered to pretreat at least 330 minutes prior to the 2C-B form disclosed herein including those described in Table 18. In some embodiments, the serotonin receptor modulator is pruvanserin and the psychedelic is 2C-B, wherein the pruvanserin is administered to pretreat at least 360 minutes prior to the 2C-B form disclosed herein including those described in Table 18. In some preferred embodiments, the serotonin receptor modulator is pruvanserin and the psychedelic is 2C-B, wherein pruvanserin is administered to pretreat between about 60 minutes and about 180 minutes prior to the administration of the 2C-B form disclosed herein including those described in Table 18.

In some embodiments, the serotonin receptor modulator is flibanserin and the psychedelic is 2C-B, wherein the flibanserin is administered to pretreat at least 15 minutes prior to the administration of the 2C-B form disclosed herein including those described in Table 18. In some embodiments, the serotonin receptor modulator is flibanserin and the psychedelic is 2C-B, wherein the flibanserin is administered to pretreat at least 30 minutes prior to the 2C-B form disclosed herein including those described in Table 18. In some embodiments, the serotonin receptor modulator is flibanserin and the psychedelic is 2C-B, wherein the flibanserin is administered to pretreat between at least 60 minutes and 240 minutes prior to the administration or release of the 2C-B form disclosed herein including those described in Table 18. In some embodiments, the serotonin receptor modulator is flibanserin and the psychedelic is 2C-B, wherein the flibanserin is administered to pretreat at least 90 minutes prior to the 2C-B form disclosed herein including those described in Table 18. In some embodiments, the serotonin receptor modulator is flibanserin and the psychedelic is 2C-B, wherein the flibanserin is administered to pretreat at least 120 minutes prior to the 2C-B form disclosed herein including those described in Table 18. In some embodiments, the serotonin receptor modulator is flibanserin and the psychedelic is 2C-B, wherein the flibanserin is administered to pretreat at least 150 minutes prior to the 2C-B form disclosed herein including those described in Table 18. In some embodiments, the serotonin receptor modulator is flibanserin and the psychedelic is 2C-B, wherein the flibanserin is administered to pretreat between about 15 minutes and about 150 minutes prior to the 2C-B form disclosed herein including those described in Table 18. In some embodiments, the serotonin receptor modulator is flibanserin and the psychedelic is 2C-B, wherein the flibanserin is administered to pretreat at least 180 minutes prior to the 2C-B form disclosed herein including those described in Table 18. In some embodiments, the serotonin receptor modulator is flibanserin and the psychedelic is 2C-B, wherein the flibanserin is administered to pretreat at least 210 minutes prior to the 2C-B form disclosed herein including those described in Table 18.

In some embodiments, the serotonin receptor modulator is flibanserin and the psychedelic is 2C-B, wherein the flibanserin is administered to pretreat at least 240 minutes prior to the 2C-B form disclosed herein including those described in Table 18. In some embodiments, the serotonin receptor modulator is flibanserin and the psychedelic is 2C-B, wherein the flibanserin is administered to pretreat at least 270 minutes prior to the 2C-B form disclosed herein including those described in Table 18. In some embodiments, the serotonin receptor modulator is flibanserin and the psychedelic is 2C-B, wherein the flibanserin is administered to pretreat at least 300 minutes prior to the 2C-B form disclosed herein including those described in Table 18. In some embodiments, the serotonin receptor modulator is flibanserin and the psychedelic is 2C-B, wherein the flibanserin is administered to pretreat at least 330 minutes prior to the 2C-B form disclosed herein including those described in Table 18. In some embodiments, the serotonin receptor modulator is flibanserin and the psychedelic is 2C-B, wherein the flibanserin is administered to pretreat at least 360 minutes prior to the 2C-B form disclosed herein including those described in Table 18. In some preferred embodiments, the serotonin receptor modulator is flibanserin and the psychedelic is 2C-B, wherein flibanserin is administered to pretreat between about 60 minutes and about 180 minutes prior to the administration of the 2C-B form disclosed herein including those described in Table 18.

In some embodiments, the serotonin receptor modulator is olanzapine and the psychedelic is 2C-B, wherein the olanzapine is administered to pretreat at least 15 minutes prior to the administration of the 2C-B form disclosed herein including those described in Table 18. In some embodiments, the serotonin receptor modulator is olanzapine and the psychedelic is 2C-B, wherein the olanzapine is administered to pretreat at least 30 minutes prior to the 2C-B form disclosed herein including those described in Table 18. In some embodiments, the serotonin receptor modulator is olanzapine and the psychedelic is 2C-B, wherein the olanzapine is administered to pretreat between at least 60 minutes and 240 minutes prior to the administration or release of the 2C-B form disclosed herein including those described in Table 18. In some embodiments, the serotonin receptor modulator is olanzapine and the psychedelic is 2C-B, wherein the olanzapine is administered to pretreat at least 90 minutes prior to the 2C-B form disclosed herein including those described in Table 18. In some embodiments, the serotonin receptor modulator is olanzapine and the psychedelic is 2C-B, wherein the olanzapine is administered to pretreat at least 120 minutes prior to the 2C-B form disclosed herein including those described in Table 18. In some embodiments, the serotonin receptor modulator is olanzapine and the psychedelic is 2C-B, wherein the olanzapine is administered to pretreat at least 150 minutes prior to the 2C-B form disclosed herein including those described in Table 18. In some embodiments, the serotonin receptor modulator is olanzapine and the psychedelic is 2C-B, wherein the olanzapine is administered to pretreat between about 15 minutes and about 150 minutes prior to the 2C-B form disclosed herein including those described in Table 18. In some embodiments, the serotonin receptor modulator is olanzapine and the psychedelic is 2C-B, wherein the olanzapine is administered to pretreat at least 180 minutes prior to the 2C-B form disclosed herein including those described in Table 18. In some embodiments, the serotonin receptor modulator is olanzapine and the psychedelic is 2C-B, wherein the olanzapine is administered to pretreat at least 210 minutes prior to the 2C-B form disclosed herein including those described in Table 18.

In some embodiments, the serotonin receptor modulator is olanzapine and the psychedelic is 2C-B, wherein the olanzapine is administered to pretreat at least 240 minutes prior to the 2C-B form disclosed herein including those described in Table 18. In some embodiments, the serotonin receptor modulator is olanzapine and the psychedelic is 2C-B, wherein the olanzapine is administered to pretreat at least 270 minutes prior to the 2C-B form disclosed herein including those described in Table 18. In some embodiments, the serotonin receptor modulator is olanzapine and the psychedelic is 2C-B, wherein the olanzapine is administered to pretreat at least 300 minutes prior to the 2C-B form disclosed herein including those described in Table 18. In some embodiments, the serotonin receptor modulator is olanzapine and the psychedelic is 2C-B, wherein the olanzapine is administered to pretreat at least 330 minutes prior to the 2C-B form disclosed herein including those described in Table 18. In some embodiments, the serotonin receptor modulator is olanzapine and the psychedelic is 2C-B, wherein the olanzapine is administered to pretreat at least 360 minutes prior to the 2C-B form disclosed herein including those described in Table 18. In some preferred embodiments, the serotonin receptor modulator is olanzapine and the psychedelic is 2C-B, wherein olanzapine is administered to pretreat between about 60 minutes and about 180 minutes prior to the administration of the 2C-B form disclosed herein including those described in Table 18.

In some embodiments, the serotonin receptor modulator is quetiapine and the psychedelic is 2C-B, wherein the quetiapine is administered to pretreat at least 15 minutes prior to the administration of the 2C-B form disclosed herein including those described in Table 18. In some embodiments, the serotonin receptor modulator is quetiapine and the psychedelic is 2C-B, wherein the quetiapine is administered to pretreat at least 30 minutes prior to the 2C-B form disclosed herein including those described in Table 18. In some embodiments, the serotonin receptor modulator is quetiapine and the psychedelic is 2C-B, wherein the quetiapine is administered to pretreat between at least 60 minutes and 240 minutes prior to the administration or release of the 2C-B form disclosed herein including those described in Table 18. In some embodiments, the serotonin receptor modulator is quetiapine and the psychedelic is 2C-B, wherein the quetiapine is administered to pretreat at least 90 minutes prior to the 2C-B form disclosed herein including those described in Table 18. In some embodiments, the serotonin receptor modulator is quetiapine and the psychedelic is 2C-B, wherein the quetiapine is administered to pretreat at least 120 minutes prior to the 2C-B form disclosed herein including those described in Table 18. In some embodiments, the serotonin receptor modulator is quetiapine and the psychedelic is 2C-B, wherein the quetiapine is administered to pretreat at least 150 minutes prior to the 2C-B form disclosed herein including those described in Table 18. In some embodiments, the serotonin receptor modulator is quetiapine and the psychedelic is 2C-B, wherein the quetiapine is administered to pretreat between about 15 minutes and about 150 minutes prior to the 2C-B form disclosed herein including those described in Table 18. In some embodiments, the serotonin receptor modulator is quetiapine and the psychedelic is 2C-B, wherein the quetiapine is administered to pretreat at least 180 minutes prior to the 2C-B form disclosed herein including those described in Table 18. In some embodiments, the serotonin receptor modulator is quetiapine and the psychedelic is 2C-B, wherein the quetiapine is administered to pretreat at least 210 minutes prior to the 2C-B form disclosed herein including those described in Table 18.

In some embodiments, the serotonin receptor modulator is quetiapine and the psychedelic is 2C-B, wherein the quetiapine is administered to pretreat at least 240 minutes prior to the 2C-B form disclosed herein including those described in Table 18. In some embodiments, the serotonin receptor modulator is quetiapine and the psychedelic is 2C-B, wherein the quetiapine is administered to pretreat at least 270 minutes prior to the 2C-B form disclosed herein including those described in Table 18. In some embodiments, the serotonin receptor modulator is quetiapine and the psychedelic is 2C-B, wherein the quetiapine is administered to pretreat at least 300 minutes prior to the 2C-B form disclosed herein including those described in Table 18. In some embodiments, the serotonin receptor modulator is quetiapine and the psychedelic is 2C-B, wherein the quetiapine is administered to pretreat at least 330 minutes prior to the 2C-B form disclosed herein including those described in Table 18. In some embodiments, the serotonin receptor modulator is quetiapine and the psychedelic is 2C-B, wherein the quetiapine is administered to pretreat at least 360 minutes prior to the 2C-B form disclosed herein including those described in Table 18. In some preferred embodiments, the serotonin receptor modulator is quetiapine and the psychedelic is 2C-B, wherein quetiapine is administered to pretreat between about 60 minutes and about 180 minutes prior to the administration of the 2C-B form disclosed herein including those described in Table 18.

In some embodiments, the serotonin receptor modulator is risperidone and the psychedelic is 2C-B, wherein the risperidone is administered to pretreat at least 15 minutes prior to the administration of the 2C-B form disclosed herein including those described in Table 18. In some embodiments, the serotonin receptor modulator is risperidone and the psychedelic is 2C-B, wherein the risperidone is administered to pretreat at least 30 minutes prior to the 2C-B form disclosed herein including those described in Table 18. In some embodiments, the serotonin receptor modulator is risperidone and the psychedelic is 2C-B, wherein the risperidone is administered to pretreat between at least 60 minutes and 240 minutes prior to the administration or release of the 2C-B form disclosed herein including those described in Table 18. In some embodiments, the serotonin receptor modulator is risperidone and the psychedelic is 2C-B, wherein the risperidone is administered to pretreat at least 90 minutes prior to the 2C-B form disclosed herein including those described in Table 18. In some embodiments, the serotonin receptor modulator is risperidone and the psychedelic is 2C-B, wherein the risperidone is administered to pretreat at least 120 minutes prior to the 2C-B form disclosed herein including those described in Table 18. In some embodiments, the serotonin receptor modulator is risperidone and the psychedelic is 2C-B, wherein the risperidone is administered to pretreat at least 150 minutes prior to the 2C-B form disclosed herein including those described in Table 18. In some embodiments, the serotonin receptor modulator is risperidone and the psychedelic is 2C-B, wherein the risperidone is administered to pretreat between about 15 minutes and about 150 minutes prior to the 2C-B form disclosed herein including those described in Table 18. In some embodiments, the serotonin receptor modulator is risperidone and the psychedelic is 2C-B, wherein the risperidone is administered to pretreat at least 180 minutes prior to the 2C-B form disclosed herein including those described in Table 18. In some embodiments, the serotonin receptor modulator is risperidone and the psychedelic is 2C-B, wherein the risperidone is administered to pretreat at least 210 minutes prior to the 2C-B form disclosed herein including those described in Table 18.

In some embodiments, the serotonin receptor modulator is risperidone and the psychedelic is 2C-B, wherein the risperidone is administered to pretreat at least 240 minutes prior to the 2C-B form disclosed herein including those described in Table 18. In some embodiments, the serotonin receptor modulator is risperidone and the psychedelic is 2C-B, wherein the risperidone is administered to pretreat at least 270 minutes prior to the 2C-B form disclosed herein including those described in Table 18. In some embodiments, the serotonin receptor modulator is risperidone and the psychedelic is 2C-B, wherein the risperidone is administered to pretreat at least 300 minutes prior to the 2C-B form disclosed herein including those described in Table 18. In some embodiments, the serotonin receptor modulator is risperidone and the psychedelic is 2C-B, wherein the risperidone is administered to pretreat at least 330 minutes prior to the 2C-B form disclosed herein including those described in Table 18. In some embodiments, the serotonin receptor modulator is risperidone and the psychedelic is 2C-B, wherein the risperidone is administered to pretreat at least 360 minutes prior to the 2C-B form disclosed herein including those described in Table 18. In some preferred embodiments, the serotonin receptor modulator is risperidone and the psychedelic is 2C-B, wherein risperidone is administered to pretreat between about 60 minutes and about 180 minutes prior to the administration of the 2C-B form disclosed herein including those described in Table 18.

In some embodiments, the serotonin receptor modulator is eplivanserin and the psychedelic is 2C-B tartrate Form 1, wherein the eplivanserin is administered to pretreat at least 15 minutes prior to the administration of the 2C-B tartrate Form 1. In some embodiments, the serotonin receptor modulator is eplivanserin and the psychedelic is 2C-B tartrate Form 1, wherein the eplivanserin is administered to pretreat between at least 30 minutes prior and 360 minutes prior to the release or administration of the 2C-B tartrate Form 1. In some embodiments, the serotonin receptor modulator is eplivanserin and the psychedelic is 2C-B tartrate Form 1, wherein the eplivanserin is administered to pretreat between at least 60 minutes prior and 360 minutes prior to the release or administration of the 2C-B tartrate Form 1. In some embodiments, the serotonin receptor modulator is eplivanserin and the psychedelic is 2C-B tartrate Form 1, wherein the eplivanserin is administered to pretreat between at least 90 minutes and 240 minutes prior to the 2C-B tartrate Form 1. In some embodiments, the serotonin receptor modulator is eplivanserin and the psychedelic is 2C-B tartrate Form 1, wherein the eplivanserin is administered to pretreat at least 120 minutes prior to the 2C-B tartrate Form 1.

In some embodiments, the serotonin receptor modulator is eplivanserin and the psychedelic is 2C-B tartrate Form 1, wherein the eplivanserin is administered to pretreat at least 150 minutes prior to the 2C-B tartrate Form 1. In some embodiments, the serotonin receptor modulator is eplivanserin and the psychedelic is 2C-B tartrate Form 1, wherein the eplivanserin is administered to pretreat between about 15 minutes and about 150 minutes prior to the 2C-B tartrate Form 1. In some embodiments, the serotonin receptor modulator is eplivanserin and the psychedelic is 2C-B tartrate Form 1, wherein the eplivanserin is administered to pretreat at least 180 minutes prior to the 2C-B tartrate Form 1. In some embodiments, the serotonin receptor modulator is eplivanserin and the psychedelic is 2C-B tartrate Form 1, wherein the eplivanserin is administered to pretreat at least 210 minutes prior to the 2C-B tartrate Form 1. In some embodiments, the serotonin receptor modulator is eplivanserin and the psychedelic is 2C-B tartrate Form 1, wherein the eplivanserin is administered to pretreat at least 240 minutes prior to the 2C-B tartrate Form 1.

In some embodiments, the serotonin receptor modulator is eplivanserin and the psychedelic is 2C-B tartrate Form 1, wherein the eplivanserin is administered to pretreat at least 270 minutes prior to 2C-B tartrate Form 1. In some embodiments, the serotonin receptor modulator is eplivanserin and the psychedelic is 2C-B tartrate Form 1, wherein the eplivanserin is administered to pretreat at least 300 minutes prior to the 2C-B tartrate Form 1. In some embodiments, the serotonin receptor modulator is eplivanserin and the psychedelic is 2C-B tartrate Form 1, wherein the eplivanserin is administered to pretreat at least 330 minutes prior to the 2C-B tartrate Form 1. In some embodiments, the serotonin receptor modulator is eplivanserin and the psychedelic is 2C-B tartrate Form 1, wherein the eplivanserin is administered to pretreat at least 360 minutes prior to the 2C-B tartrate Form 1.

In some preferred embodiments, the serotonin receptor modulator is eplivanserin and the psychedelic is 2C-B tartrate Form 1, wherein eplivanserin is administered to pretreat between about 60 minutes and about 180 minutes prior to the administration of the 2C-B tartrate Form 1.

In some embodiments, the serotonin receptor modulator is volinanserin and the psychedelic is 2C-B tartrate Form 1, wherein the volinanserin is administered to pretreat a subject between at least 15 minutes and 360 minutes prior to the administration or release of the 2C-B tartrate Form 1. In some embodiments, the serotonin receptor modulator is volinanserin and the psychedelic is 2C-B tartrate Form 1, wherein the volinanserin is administered to pretreat between at least 30 minutes and 360 minutes prior to the administration or release of the 2C-B tartrate Form 1. In some embodiments, the serotonin receptor modulator is volinanserin and the psychedelic is 2C-B tartrate Form 1, wherein the volinanserin is administered to pretreat between at least 60 minutes and 240 minutes prior to the administration or release of the 2C-B tartrate Form 1. In some embodiments, the serotonin receptor modulator is volinanserin and the psychedelic is 2C-B tartrate Form 1, wherein the volinanserin is administered to pretreat at least 90 minutes prior to the 2C-B tartrate Form 1. In some embodiments, the serotonin receptor modulator is volinanserin and the psychedelic is 2C-B tartrate Form 1, wherein the volinanserin is administered to pretreat at least 120 minutes prior to the 2C-B tartrate Form 1.

In some embodiments, the serotonin receptor modulator is volinanserin and the psychedelic is 2C-B tartrate Form 1, wherein the volinanserin is administered to pretreat at least 150 minutes prior to the 2C-B tartrate Form 1. In some embodiments, the serotonin receptor modulator is volinanserin and the psychedelic is 2C-B tartrate Form 1, wherein the volinanserin is administered to pretreat between about 15 minutes and about 150 minutes prior to the 2C-B tartrate Form 1. In some embodiments, the serotonin receptor modulator is volinanserin and the psychedelic is 2C-B tartrate Form 1, wherein the volinanserin is administered to pretreat at least 180 minutes prior to the 2C-B tartrate Form 1. In some embodiments, the serotonin receptor modulator is volinanserin and the psychedelic is 2C-B tartrate Form 1, wherein the volinanserin is administered to pretreat at least 210 minutes prior to the 2C-B tartrate Form 1. In some embodiments, the serotonin receptor modulator is volinanserin and the psychedelic is 2C-B tartrate Form 1, wherein the volinanserin is administered to pretreat at least 240 minutes prior to the 2C-B tartrate Form 1. In some embodiments, the serotonin receptor modulator is volinanserin and the psychedelic is 2C-B tartrate Form 1, wherein the volinanserin is administered to pretreat at least 270 minutes prior to the 2C-B tartrate Form 1. In some embodiments, the serotonin receptor modulator is volinanserin and the psychedelic is 2C-B tartrate Form 1, wherein the volinanserin is administered to pretreat at least 300 minutes prior to the 2C-B tartrate Form 1. In some embodiments, the serotonin receptor modulator is volinanserin and the psychedelic is 2C-B tartrate Form 1, wherein the volinanserin is administered to pretreat at least 330 minutes prior to the 2C-B tartrate Form 1. In some embodiments, the serotonin receptor modulator is volinanserin and the psychedelic is 2C-B tartrate Form 1, wherein the volinanserin is administered to pretreat at least 360 minutes prior to the 2C-B tartrate Form 1. In some preferred embodiments, the serotonin receptor modulator is volinanserin and the psychedelic is 2C-B tartrate Form 1, wherein volinanserin is administered to pretreat between about 60 minutes and about 180 minutes prior to the administration of the 2C-B tartrate Form 1.

In some embodiments, the serotonin receptor modulator is ketanserin and the psychedelic is 2C-B tartrate Form 1, wherein the ketanserin is administered to pretreat at least 15 minutes prior to the administration of the 2C-B tartrate Form 1. In some embodiments, the serotonin receptor modulator is ketanserin and the psychedelic is 2C-B tartrate Form 1, wherein the ketanserin is administered to pretreat between at least 30 minutes and 360 minutes prior to the administration or release of the 2C-B tartrate Form 1. In some embodiments, the serotonin receptor modulator is ketanserin and the psychedelic is 2C-B tartrate Form 1, wherein the ketanserin is administered to pretreat between at least 60 minutes and 240 minutes prior to the administration or release of the 2C-B tartrate Form 1. In some embodiments, the serotonin receptor modulator is ketanserin and the psychedelic is 2C-B tartrate Form 1, wherein the ketanserin is administered to pretreat at least 90 minutes prior to the 2C-B tartrate Form 1. In some embodiments, the serotonin receptor modulator is ketanserin and the psychedelic is 2C-B tartrate Form 1, wherein the ketanserin is administered to pretreat at least 120 minutes prior to the 2C-B tartrate Form 1. In some embodiments, the serotonin receptor modulator is ketanserin and the psychedelic is 2C-B tartrate Form 1, wherein the ketanserin is administered to pretreat at least 150 minutes prior to the 2C-B tartrate Form 1. In some embodiments, the serotonin receptor modulator is ketanserin and the psychedelic is 2C-B tartrate Form 1, wherein the ketanserin is administered to pretreat between about 15 minutes and about 150 minutes prior to the 2C-B tartrate Form 1. In some embodiments, the serotonin receptor modulator is ketanserin and the psychedelic is 2C-B tartrate Form 1, wherein the ketanserin is administered to pretreat at least 180 minutes prior to the 2C-B tartrate Form 1. In some embodiments, the serotonin receptor modulator is ketanserin and the psychedelic is 2C-B tartrate Form 1, wherein the ketanserin is administered to pretreat at least 210 minutes prior to the 2C-B tartrate Form 1. In some embodiments, the serotonin receptor modulator is ketanserin and the psychedelic is 2C-B tartrate Form 1, wherein the ketanserin is administered to pretreat at least 240 minutes prior to the 2C-B tartrate Form 1. In some embodiments, the serotonin receptor modulator is ketanserin and the psychedelic is 2C-B tartrate Form 1, wherein the ketanserin is administered to pretreat at least 270 minutes prior to the 2C-B tartrate Form 1. In some embodiments, the serotonin receptor modulator is ketanserin and the psychedelic is 2C-B tartrate Form 1, wherein the ketanserin is administered to pretreat at least 300 minutes prior to the 2C-B tartrate Form 1. In some embodiments, the serotonin receptor modulator is ketanserin and the psychedelic is 2C-B tartrate Form 1, wherein the ketanserin is administered to pretreat at least 330 minutes prior to the 2C-B tartrate Form 1. In some embodiments, the serotonin receptor modulator is ketanserin and the psychedelic is 2C-B tartrate Form 1, wherein the ketanserin is administered to pretreat at least 360 minutes prior to the 2C-B tartrate Form 1. In some preferred embodiments, the serotonin receptor modulator is ketanserin and the psychedelic is 2C-B tartrate Form 1, wherein ketanserin is administered to pretreat between about 60 minutes and about 180 minutes prior to the administration of the 2C-B tartrate Form 1.

In some embodiments, the serotonin receptor modulator is ritanserin and the psychedelic is 2C-B tartrate Form 1, wherein the ritanserin is administered to pretreat at least 15 minutes prior to the administration of the 2C-B tartrate Form 1. In some embodiments, the serotonin receptor modulator is ritanserin and the psychedelic is 2C-B tartrate Form 1, wherein the ritanserin is administered to pretreat at least 30 minutes prior to the 2C-B tartrate Form 1. In some embodiments, the serotonin receptor modulator is ritanserin and the psychedelic is 2C-B tartrate Form 1, wherein the ritanserin is administered to pretreat between at least 60 minutes and 240 minutes prior to the administration or release of the 2C-B tartrate Form 1. In some embodiments, the serotonin receptor modulator is ritanserin and the psychedelic is 2C-B tartrate Form 1, wherein the ritanserin is administered to pretreat at least 90 minutes prior to the 2C-B tartrate Form 1. In some embodiments, the serotonin receptor modulator is ritanserin and the psychedelic is 2C-B tartrate Form 1, wherein the ritanserin is administered to pretreat at least 120 minutes prior to the 2C-B tartrate Form 1. In some embodiments, the serotonin receptor modulator is ritanserin and the psychedelic is 2C-B tartrate Form 1, wherein the ritanserin is administered to pretreat at least 150 minutes prior to the 2C-B tartrate Form 1. In some embodiments, the serotonin receptor modulator is ritanserin and the psychedelic is 2C-B tartrate Form 1, wherein the ritanserin is administered to pretreat between about 15 minutes and about 150 minutes prior to the 2C-B tartrate Form 1. In some embodiments, the serotonin receptor modulator is ritanserin and the psychedelic is 2C-B tartrate Form 1, wherein the ritanserin is administered to pretreat at least 180 minutes prior to the 2C-B tartrate Form 1. In some embodiments, the serotonin receptor modulator is ritanserin and the psychedelic is 2C-B tartrate Form 1, wherein the ritanserin is administered to pretreat at least 210 minutes prior to the 2C-B tartrate Form 1. In some embodiments, the serotonin receptor modulator is ritanserin and the psychedelic is 2C-B tartrate Form 1, wherein the ritanserin is administered to pretreat at least 240 minutes prior to the 2C-B tartrate Form 1. In some embodiments, the serotonin receptor modulator is ritanserin and the psychedelic is 2C-B tartrate Form 1, wherein the ritanserin is administered to pretreat at least 270 minutes prior to the 2C-B tartrate Form 1. In some embodiments, the serotonin receptor modulator is ritanserin and the psychedelic is 2C-B tartrate Form 1, wherein the ritanserin is administered to pretreat at least 300 minutes prior to the 2C-B tartrate Form 1. In some embodiments, the serotonin receptor modulator is ritanserin and the psychedelic is 2C-B tartrate Form 1, wherein the ritanserin is administered to pretreat at least 330 minutes prior to the 2C-B tartrate Form 1. In some embodiments, the serotonin receptor modulator is ritanserin and the psychedelic is 2C-B tartrate Form 1, wherein the ritanserin is administered to pretreat at least 360 minutes prior to the 2C-B tartrate Form 1. In some preferred embodiments, the serotonin receptor modulator is ritanserin and the psychedelic is 2C-B tartrate Form 1, wherein ritanserin is administered to pretreat between about 60 minutes and about 180 minutes prior to the administration of the 2C-B tartrate Form 1.

In some embodiments, the serotonin receptor modulator is pimavanserin and the psychedelic is 2C-B tartrate Form 1, wherein the pimavanserin is administered to pretreat at least 15 minutes prior to the administration of the 2C-B tartrate Form 1. In some embodiments, the serotonin receptor modulator is pimavanserin and the psychedelic is 2C-B tartrate Form 1, wherein the pimavanserin is administered to pretreat at least 30 minutes prior to the 2C-B tartrate Form 1. In some embodiments, the serotonin receptor modulator is pimavanserin and the psychedelic is 2C-B tartrate Form 1, wherein the pimavanserin is administered to pretreat between at least 60 minutes and 240 minutes prior to the administration or release of the 2C-B tartrate Form 1. In some embodiments, the serotonin receptor modulator is pimavanserin and the psychedelic is 2C-B tartrate Form 1, wherein the pimavanserin is administered to pretreat at least 90 minutes prior to the 2C-B tartrate Form 1. In some embodiments, the serotonin receptor modulator is pimavanserin and the psychedelic is 2C-B tartrate Form 1, wherein the pimavanserin is administered to pretreat at least 120 minutes prior to the 2C-B tartrate Form 1. In some embodiments, the serotonin receptor modulator is pimavanserin and the psychedelic is 2C-B tartrate Form 1, wherein the pimavanserin is administered to pretreat at least 150 minutes prior to the 2C-B tartrate Form 1. In some embodiments, the serotonin receptor modulator is pimavanserin and the psychedelic is 2C-B tartrate Form 1, wherein the pimavanserin is administered to pretreat between about 15 minutes and about 150 minutes prior to the 2C-B tartrate Form 1. In some embodiments, the serotonin receptor modulator is pimavanserin and the psychedelic is 2C-B tartrate Form 1, wherein the pimavanserin is administered to pretreat at least 180 minutes prior to the 2C-B tartrate Form 1. In some embodiments, the serotonin receptor modulator is pimavanserin and the psychedelic is 2C-B tartrate Form 1, wherein the pimavanserin is administered to pretreat at least 210 minutes prior to the 2C-B tartrate Form 1. In some embodiments, the serotonin receptor modulator is pimavanserin and the psychedelic is 2C-B tartrate Form 1, wherein the pimavanserin is administered to pretreat at least 240 minutes prior to the 2C-B tartrate Form 1. In some embodiments, the serotonin receptor modulator is pimavanserin and the psychedelic is 2C-B tartrate Form 1, wherein the pimavanserin is administered to pretreat at least 270 minutes prior to the 2C-B tartrate Form 1. In some embodiments, the serotonin receptor modulator is pimavanserin and the psychedelic is 2C-B tartrate Form 1, wherein the pimavanserin is administered to pretreat at least 300 minutes prior to the 2C-B tartrate Form 1. In some embodiments, the serotonin receptor modulator is pimavanserin and the psychedelic is 2C-B tartrate Form 1, wherein the pimavanserin is administered to pretreat at least 330 minutes prior to the 2C-B tartrate Form 1. In some embodiments, the serotonin receptor modulator is pimavanserin and the psychedelic is 2C-B tartrate Form 1, wherein the pimavanserin is administered to pretreat at least 360 minutes prior to the 2C-B tartrate Form 1. In some preferred embodiments, the serotonin receptor modulator is pimavanserin and the psychedelic is 2C-B tartrate Form 1, wherein pimavanserin is administered to pretreat between about 60 minutes and about 180 minutes prior to the administration of the 2C-B tartrate Form 1.

In some embodiments, the serotonin receptor modulator is nelotanserin and the psychedelic is 2C-B tartrate Form 1, wherein the nelotanserin is administered to pretreat at least 15 minutes prior to the administration of the 2C-B tartrate Form 1. In some embodiments, the serotonin receptor modulator is nelotanserin and the psychedelic is 2C-B tartrate Form 1, wherein the nelotanserin is administered to pretreat at least 30 minutes prior to the 2C-B tartrate Form 1. In some embodiments, the serotonin receptor modulator is nelotanserin and the psychedelic is 2C-B tartrate Form 1, wherein the nelotanserin is administered to pretreat between at least 60 minutes and 240 minutes prior to the administration or release of the 2C-B tartrate Form 1. In some embodiments, the serotonin receptor modulator is nelotanserin and the psychedelic is 2C-B tartrate Form 1, wherein the nelotanserin is administered to pretreat at least 90 minutes prior to the 2C-B tartrate Form 1. In some embodiments, the serotonin receptor modulator is nelotanserin and the psychedelic is 2C-B tartrate Form 1, wherein the nelotanserin is administered to pretreat at least 120 minutes prior to the 2C-B tartrate Form 1.

In some embodiments, the serotonin receptor modulator is nelotanserin and the psychedelic is 2C-B tartrate Form 1, wherein the nelotanserin is administered to pretreat at least 150 minutes prior to the 2C-B tartrate Form 1. In some embodiments, the serotonin receptor modulator is nelotanserin and the psychedelic is 2C-B tartrate Form 1, wherein the nelotanserin is administered to pretreat between about 15 minutes and about 150 minutes prior to the 2C-B tartrate Form 1. In some embodiments, the serotonin receptor modulator is nelotanserin and the psychedelic is 2C-B tartrate Form 1, wherein the nelotanserin is administered to pretreat at least 180 minutes prior to the 2C-B tartrate Form 1. In some embodiments, the serotonin receptor modulator is nelotanserin and the psychedelic is 2C-B tartrate Form 1, wherein the nelotanserin is administered to pretreat at least 210 minutes prior to the 2C-B tartrate Form 1. In some embodiments, the serotonin receptor modulator is nelotanserin and the psychedelic is 2C-B tartrate Form 1, wherein the nelotanserin is administered to pretreat at least 240 minutes prior to the 2C-B tartrate Form 1. In some embodiments, the serotonin receptor modulator is nelotanserin and the psychedelic is 2C-B tartrate Form 1, wherein the nelotanserin is administered to pretreat at least 270 minutes prior to the 2C-B tartrate Form 1.

In some embodiments, the serotonin receptor modulator is nelotanserin and the psychedelic is 2C-B tartrate Form 1, wherein the nelotanserin is administered to pretreat at least 300 minutes prior to the 2C-B tartrate Form 1. In some embodiments, the serotonin receptor modulator is nelotanserin and the psychedelic is 2C-B tartrate Form 1, wherein the nelotanserin is administered to pretreat at least 330 minutes prior to the 2C-B tartrate Form 1. In some embodiments, the serotonin receptor modulator is nelotanserin and the psychedelic is 2C-B tartrate Form 1, wherein the nelotanserin is administered to pretreat at least 360 minutes prior to the 2C-B tartrate Form 1. In some preferred embodiments, the serotonin receptor modulator is nelotanserin and the psychedelic is 2C-B tartrate Form 1, wherein nelotanserin is administered to pretreat between about 60 minutes and about 180 minutes prior to the administration of the 2C-B tartrate Form 1.

In some embodiments, the serotonin receptor modulator is pruvanserin and the psychedelic is 2C-B tartrate Form 1, wherein the pruvanserin is administered to pretreat at least 15 minutes prior to the administration of the 2C-B tartrate Form 1. In some embodiments, the serotonin receptor modulator is pruvanserin and the psychedelic is 2C-B tartrate Form 1, wherein the pruvanserin is administered to pretreat at least 30 minutes prior to the 2C-B tartrate Form 1. In some embodiments, the serotonin receptor modulator is pruvanserin and the psychedelic is 2C-B tartrate Form 1, wherein the pruvanserin is administered to pretreat between at least 60 minutes and 240 minutes prior to the administration or release of the 2C-B tartrate Form 1. In some embodiments, the serotonin receptor modulator is pruvanserin and the psychedelic is 2C-B tartrate Form 1, wherein the pruvanserin is administered to pretreat at least 90 minutes prior to the 2C-B tartrate Form 1. In some embodiments, the serotonin receptor modulator is pruvanserin and the psychedelic is 2C-B tartrate Form 1, wherein the pruvanserin is administered to pretreat at least 120 minutes prior to the 2C-B tartrate Form 1. In some embodiments, the serotonin receptor modulator is pruvanserin and the psychedelic is 2C-B tartrate Form 1, wherein the pruvanserin is administered to pretreat at least 150 minutes prior to the 2C-B tartrate Form 1. In some embodiments, the serotonin receptor modulator is pruvanserin and the psychedelic is 2C-B tartrate Form 1, wherein the pruvanserin is administered to pretreat between about 15 minutes and about 150 minutes prior to the 2C-B tartrate Form 1. In some embodiments, the serotonin receptor modulator is pruvanserin and the psychedelic is 2C-B tartrate Form 1, wherein the pruvanserin is administered to pretreat at least 180 minutes prior to the 2C-B tartrate Form 1. In some embodiments, the serotonin receptor modulator is pruvanserin and the psychedelic is 2C-B tartrate Form 1, wherein the pruvanserin is administered to pretreat at least 210 minutes prior to the 2C-B tartrate Form 1.

In some embodiments, the serotonin receptor modulator is pruvanserin and the psychedelic is 2C-B tartrate Form 1, wherein the pruvanserin is administered to pretreat at least 240 minutes prior to the 2C-B tartrate Form 1. In some embodiments, the serotonin receptor modulator is pruvanserin and the psychedelic is 2C-B tartrate Form 1, wherein the pruvanserin is administered to pretreat at least 270 minutes prior to the 2C-B tartrate Form 1. In some embodiments, the serotonin receptor modulator is pruvanserin and the psychedelic is 2C-B tartrate Form 1, wherein the pruvanserin is administered to pretreat at least 300 minutes prior to the 2C-B tartrate Form 1. In some embodiments, the serotonin receptor modulator is pruvanserin and the psychedelic is 2C-B tartrate Form 1, wherein the pruvanserin is administered to pretreat at least 330 minutes prior to the 2C-B tartrate Form 1. In some embodiments, the serotonin receptor modulator is pruvanserin and the psychedelic is 2C-B tartrate Form 1, wherein the pruvanserin is administered to pretreat at least 360 minutes prior to the 2C-B tartrate Form 1. In some preferred embodiments, the serotonin receptor modulator is pruvanserin and the psychedelic is 2C-B tartrate Form 1, wherein pruvanserin is administered to pretreat between about 60 minutes and about 180 minutes prior to the administration of the 2C-B tartrate Form 1.

In some embodiments, the serotonin receptor modulator is flibanserin and the psychedelic is 2C-B tartrate Form 1, wherein the flibanserin is administered to pretreat at least 15 minutes prior to the administration of the 2C-B tartrate Form 1. In some embodiments, the serotonin receptor modulator is flibanserin and the psychedelic is 2C-B tartrate Form 1, wherein the flibanserin is administered to pretreat at least 30 minutes prior to the 2C-B tartrate Form 1. In some embodiments, the serotonin receptor modulator is flibanserin and the psychedelic is 2C-B tartrate Form 1, wherein the flibanserin is administered to pretreat between at least 60 minutes and 240 minutes prior to the administration or release of the 2C-B tartrate Form 1. In some embodiments, the serotonin receptor modulator is flibanserin and the psychedelic is 2C-B tartrate Form 1, wherein the flibanserin is administered to pretreat at least 90 minutes prior to the 2C-B tartrate Form 1. In some embodiments, the serotonin receptor modulator is flibanserin and the psychedelic is 2C-B tartrate Form 1, wherein the flibanserin is administered to pretreat at least 120 minutes prior to the 2C-B tartrate Form 1. In some embodiments, the serotonin receptor modulator is flibanserin and the psychedelic is 2C-B tartrate Form 1, wherein the flibanserin is administered to pretreat at least 150 minutes prior to the 2C-B tartrate Form 1. In some embodiments, the serotonin receptor modulator is flibanserin and the psychedelic is 2C-B tartrate Form 1, wherein the flibanserin is administered to pretreat between about 15 minutes and about 150 minutes prior to the 2C-B tartrate Form 1. In some embodiments, the serotonin receptor modulator is flibanserin and the psychedelic is 2C-B tartrate Form 1, wherein the flibanserin is administered to pretreat at least 180 minutes prior to the 2C-B tartrate Form 1. In some embodiments, the serotonin receptor modulator is flibanserin and the psychedelic is 2C-B tartrate Form 1, wherein the flibanserin is administered to pretreat at least 210 minutes prior to the 2C-B tartrate Form 1.

In some embodiments, the serotonin receptor modulator is flibanserin and the psychedelic is 2C-B tartrate Form 1, wherein the flibanserin is administered to pretreat at least 240 minutes prior to the 2C-B tartrate Form 1. In some embodiments, the serotonin receptor modulator is flibanserin and the psychedelic is 2C-B tartrate Form 1, wherein the flibanserin is administered to pretreat at least 270 minutes prior to the 2C-B tartrate Form 1. In some embodiments, the serotonin receptor modulator is flibanserin and the psychedelic is 2C-B tartrate Form 1, wherein the flibanserin is administered to pretreat at least 300 minutes prior to the 2C-B tartrate Form 1. In some embodiments, the serotonin receptor modulator is flibanserin and the psychedelic is 2C-B tartrate Form 1, wherein the flibanserin is administered to pretreat at least 330 minutes prior to the 2C-B tartrate Form 1. In some embodiments, the serotonin receptor modulator is flibanserin and the psychedelic is 2C-B tartrate Form 1, wherein the flibanserin is administered to pretreat at least 360 minutes prior to the 2C-B tartrate Form 1. In some preferred embodiments, the serotonin receptor modulator is flibanserin and the psychedelic is 2C-B tartrate Form 1, wherein flibanserin is administered to pretreat between about 60 minutes and about 180 minutes prior to the administration of the 2C-B tartrate Form 1.

In some embodiments, the serotonin receptor modulator is olanzapine and the psychedelic is 2C-B tartrate Form 1, wherein the olanzapine is administered to pretreat at least 15 minutes prior to the administration of the 2C-B tartrate Form 1. In some embodiments, the serotonin receptor modulator is olanzapine and the psychedelic is 2C-B tartrate Form 1, wherein the olanzapine is administered to pretreat at least 30 minutes prior to the 2C-B tartrate Form 1. In some embodiments, the serotonin receptor modulator is olanzapine and the psychedelic is 2C-B tartrate Form 1, wherein the olanzapine is administered to pretreat between at least 60 minutes and 240 minutes prior to the administration or release of the 2C-B tartrate Form 1. In some embodiments, the serotonin receptor modulator is olanzapine and the psychedelic is 2C-B tartrate Form 1, wherein the olanzapine is administered to pretreat at least 90 minutes prior to the 2C-B tartrate Form 1. In some embodiments, the serotonin receptor modulator is olanzapine and the psychedelic is 2C-B tartrate Form 1, wherein the olanzapine is administered to pretreat at least 120 minutes prior to the 2C-B tartrate Form 1. In some embodiments, the serotonin receptor modulator is olanzapine and the psychedelic is 2C-B tartrate Form 1, wherein the olanzapine is administered to pretreat at least 150 minutes prior to the 2C-B tartrate Form 1. In some embodiments, the serotonin receptor modulator is olanzapine and the psychedelic is 2C-B tartrate Form 1, wherein the olanzapine is administered to pretreat between about 15 minutes and about 150 minutes prior to the 2C-B tartrate Form 1. In some embodiments, the serotonin receptor modulator is olanzapine and the psychedelic is 2C-B tartrate Form 1, wherein the olanzapine is administered to pretreat at least 180 minutes prior to the 2C-B tartrate Form 1. In some embodiments, the serotonin receptor modulator is olanzapine and the psychedelic is 2C-B tartrate Form 1, wherein the olanzapine is administered to pretreat at least 210 minutes prior to the 2C-B tartrate Form 1.

In some embodiments, the serotonin receptor modulator is olanzapine and the psychedelic is 2C-B tartrate Form 1, wherein the olanzapine is administered to pretreat at least 240 minutes prior to the 2C-B tartrate Form 1. In some embodiments, the serotonin receptor modulator is olanzapine and the psychedelic is 2C-B tartrate Form 1, wherein the olanzapine is administered to pretreat at least 270 minutes prior to the 2C-B tartrate Form 1. In some embodiments, the serotonin receptor modulator is olanzapine and the psychedelic is 2C-B tartrate Form 1, wherein the olanzapine is administered to pretreat at least 300 minutes prior to the 2C-B tartrate Form 1. In some embodiments, the serotonin receptor modulator is olanzapine and the psychedelic is 2C-B tartrate Form 1, wherein the olanzapine is administered to pretreat at least 330 minutes prior to the 2C-B tartrate Form 1. In some embodiments, the serotonin receptor modulator is olanzapine and the psychedelic is 2C-B tartrate Form 1, wherein the olanzapine is administered to pretreat at least 360 minutes prior to the 2C-B tartrate Form 1. In some preferred embodiments, the serotonin receptor modulator is olanzapine and the psychedelic is 2C-B tartrate Form 1, wherein olanzapine is administered to pretreat between about 60 minutes and about 180 minutes prior to the administration of the 2C-B tartrate Form 1.

In some embodiments, the serotonin receptor modulator is risperidone and the psychedelic is 2C-B tartrate Form 1, wherein the risperidone is administered to pretreat at least 15 minutes prior to the administration of the 2C-B tartrate Form 1. In some embodiments, the serotonin receptor modulator is risperidone and the psychedelic is 2C-B tartrate Form 1, wherein the risperidone is administered to pretreat at least 30 minutes prior to the 2C-B tartrate Form 1. In some embodiments, the serotonin receptor modulator is risperidone and the psychedelic is 2C-B tartrate Form 1, wherein the risperidone is administered to pretreat between at least 60 minutes and 240 minutes prior to the administration or release of the 2C-B tartrate Form 1. In some embodiments, the serotonin receptor modulator is risperidone and the psychedelic is 2C-B tartrate Form 1, wherein the risperidone is administered to pretreat at least 90 minutes prior to the 2C-B tartrate Form 1. In some embodiments, the serotonin receptor modulator is risperidone and the psychedelic is 2C-B tartrate Form 1, wherein the risperidone is administered to pretreat at least 120 minutes prior to the 2C-B tartrate Form 1. In some embodiments, the serotonin receptor modulator is risperidone and the psychedelic is 2C-B tartrate Form 1, wherein the risperidone is administered to pretreat at least 150 minutes prior to the 2C-B tartrate Form 1. In some embodiments, the serotonin receptor modulator is risperidone and the psychedelic is 2C-B tartrate Form 1, wherein the risperidone is administered to pretreat between about 15 minutes and about 150 minutes prior to the 2C-B tartrate Form 1. In some embodiments, the serotonin receptor modulator is risperidone and the psychedelic is 2C-B tartrate Form 1, wherein the risperidone is administered to pretreat at least 180 minutes prior to the 2C-B tartrate Form 1. In some embodiments, the serotonin receptor modulator is risperidone and the psychedelic is 2C-B tartrate Form 1, wherein the risperidone is administered to pretreat at least 210 minutes prior to the 2C-B tartrate Form 1.

In some embodiments, the serotonin receptor modulator is risperidone and the psychedelic is 2C-B tartrate Form 1, wherein the risperidone is administered to pretreat at least 240 minutes prior to the 2C-B tartrate Form 1. In some embodiments, the serotonin receptor modulator is risperidone and the psychedelic is 2C-B tartrate Form 1, wherein the risperidone is administered to pretreat at least 270 minutes prior to the 2C-B tartrate Form 1. In some embodiments, the serotonin receptor modulator is risperidone and the psychedelic is 2C-B tartrate Form 1, wherein the risperidone is administered to pretreat at least 300 minutes prior to the 2C-B tartrate Form 1. In some embodiments, the serotonin receptor modulator is risperidone and the psychedelic is 2C-B tartrate Form 1, wherein the risperidone is administered to pretreat at least 330 minutes prior to the 2C-B tartrate Form 1. In some embodiments, the serotonin receptor modulator is risperidone and the psychedelic is 2C-B tartrate Form 1, wherein the risperidone is administered to pretreat at least 360 minutes prior to the 2C-B tartrate Form 1. In some preferred embodiments, the serotonin receptor modulator is risperidone and the psychedelic is 2C-B tartrate Form 1, wherein risperidone is administered to pretreat between about 60 minutes and about 180 minutes prior to the administration of the 2C-B tartrate Form 1.

In some embodiments, the serotonin receptor modulator is quetiapine and the psychedelic is 2C-B tartrate Form 1, wherein the quetiapine is administered to pretreat at least 15 minutes prior to the administration of the 2C-B tartrate Form 1. In some embodiments, the serotonin receptor modulator is quetiapine and the psychedelic is 2C-B tartrate Form 1, wherein the quetiapine is administered to pretreat at least 30 minutes prior to the 2C-B tartrate Form 1. In some embodiments, the serotonin receptor modulator is quetiapine and the psychedelic is 2C-B tartrate Form 1, wherein the quetiapine is administered to pretreat between at least 60 minutes and 240 minutes prior to the administration or release of the 2C-B tartrate Form 1. In some embodiments, the serotonin receptor modulator is quetiapine and the psychedelic is 2C-B tartrate Form 1, wherein the quetiapine is administered to pretreat at least 90 minutes prior to the 2C-B tartrate Form 1. In some embodiments, the serotonin receptor modulator is quetiapine and the psychedelic is 2C-B tartrate Form 1, wherein the quetiapine is administered to pretreat at least 120 minutes prior to the 2C-B tartrate Form 1. In some embodiments, the serotonin receptor modulator is quetiapine and the psychedelic is 2C-B tartrate Form 1, wherein the quetiapine is administered to pretreat at least 150 minutes prior to the 2C-B tartrate Form 1. In some embodiments, the serotonin receptor modulator is quetiapine and the psychedelic is 2C-B tartrate Form 1, wherein the quetiapine is administered to pretreat between about 15 minutes and about 150 minutes prior to the 2C-B tartrate Form 1. In some embodiments, the serotonin receptor modulator is quetiapine and the psychedelic is 2C-B tartrate Form 1, wherein the quetiapine is administered to pretreat at least 180 minutes prior to the 2C-B tartrate Form 1. In some embodiments, the serotonin receptor modulator is quetiapine and the psychedelic is 2C-B tartrate Form 1, wherein the quetiapine is administered to pretreat at least 210 minutes prior to the 2C-B tartrate Form 1.

In some embodiments, the serotonin receptor modulator is quetiapine and the psychedelic is 2C-B tartrate Form 1, wherein the quetiapine is administered to pretreat at least 240 minutes prior to the 2C-B tartrate Form 1. In some embodiments, the serotonin receptor modulator is quetiapine and the psychedelic is 2C-B tartrate Form 1, wherein the quetiapine is administered to pretreat at least 270 minutes prior to the 2C-B tartrate Form 1. In some embodiments, the serotonin receptor modulator is quetiapine and the psychedelic is 2C-B tartrate Form 1, wherein the quetiapine is administered to pretreat at least 300 minutes prior to the 2C-B tartrate Form 1.

In some embodiments, the serotonin receptor modulator is quetiapine and the psychedelic is 2C-B tartrate Form 1, wherein the quetiapine is administered to pretreat at least 330 minutes prior to the 2C-B tartrate Form 1. In some embodiments, the serotonin receptor modulator is quetiapine and the psychedelic is 2C-B tartrate Form 1, wherein the quetiapine is administered to pretreat at least 360 minutes prior to the 2C-B tartrate Form 1. In some preferred embodiments, the serotonin receptor modulator is quetiapine and the psychedelic is 2C-B tartrate Form 1, wherein quetiapine is administered to pretreat between about 60 minutes and about 180 minutes prior to the administration of the 2C-B tartrate Form 1.

In some embodiments, the serotonin receptor modulator is eplivanserin and the psychedelic is 2C-B HCl Form A, wherein the eplivanserin is administered to pretreat at least 15 minutes prior to the administration of the 2C-B HCl Form A. In some embodiments, the serotonin receptor modulator is eplivanserin and the psychedelic is 2C-B HCl Form A, wherein the eplivanserin is administered to pretreat between at least 30 minutes prior and 360 minutes prior to the release or administration of the 2C-B HCl Form A. In some embodiments, the serotonin receptor modulator is eplivanserin and the psychedelic is 2C-B HCl Form A, wherein the eplivanserin is administered to pretreat between at least 60 minutes prior and 360 minutes prior to the release or administration of the 2C-B HCl Form A. In some embodiments, the serotonin receptor modulator is eplivanserin and the psychedelic is 2C-B HCl Form A, wherein the eplivanserin is administered to pretreat between at least 90 minutes and 240 minutes prior to the 2C-B HCl Form A. In some embodiments, the serotonin receptor modulator is eplivanserin and the psychedelic is 2C-B HCl Form A, wherein the eplivanserin is administered to pretreat at least 120 minutes prior to the 2C-B HCl Form A.

In some embodiments, the serotonin receptor modulator is eplivanserin and the psychedelic is 2C-B HCl Form A, wherein the eplivanserin is administered to pretreat at least 150 minutes prior to the 2C-B HCl Form A. In some embodiments, the serotonin receptor modulator is eplivanserin and the psychedelic is 2C-B HCl Form A, wherein the eplivanserin is administered to pretreat between about 15 minutes and about 150 minutes prior to the 2C-B HCl Form A. In some embodiments, the serotonin receptor modulator is eplivanserin and the psychedelic is 2C-B HCl Form A, wherein the eplivanserin is administered to pretreat at least 180 minutes prior to the 2C-B HCl Form A. In some embodiments, the serotonin receptor modulator is eplivanserin and the psychedelic is 2C-B HCl Form A, wherein the eplivanserin is administered to pretreat at least 210 minutes prior to the 2C-B HCl Form A. In some embodiments, the serotonin receptor modulator is eplivanserin and the psychedelic is 2C-B HCl Form A, wherein the eplivanserin is administered to pretreat at least 240 minutes prior to the 2C-B HCl Form A.

In some embodiments, the serotonin receptor modulator is eplivanserin and the psychedelic is 2C-B HCl Form A, wherein the eplivanserin is administered to pretreat at least 270 minutes prior to 2C-B HCl Form A. In some embodiments, the serotonin receptor modulator is eplivanserin and the psychedelic is 2C-B HCl Form A, wherein the eplivanserin is administered to pretreat at least 300 minutes prior to the 2C-B HCl Form A. In some embodiments, the serotonin receptor modulator is eplivanserin and the psychedelic is 2C-B HCl Form A, wherein the eplivanserin is administered to pretreat at least 330 minutes prior to the 2C-B HCl Form A. In some embodiments, the serotonin receptor modulator is eplivanserin and the psychedelic is 2C-B HCl Form A, wherein the eplivanserin is administered to pretreat at least 360 minutes prior to the 2C-B HCl Form A.

In some preferred embodiments, the serotonin receptor modulator is eplivanserin and the psychedelic is 2C-B HCl Form A, wherein eplivanserin is administered to pretreat between about 60 minutes and about 180 minutes prior to the administration of the 2C-B HCl Form A.

In some embodiments, the serotonin receptor modulator is volinanserin and the psychedelic is 2C-B HCl Form A, wherein the volinanserin is administered to pretreat a subject between at least 15 minutes and 360 minutes prior to the administration or release of the 2C-B HCl Form A. In some embodiments, the serotonin receptor modulator is volinanserin and the psychedelic is 2C-B HCl Form A, wherein the volinanserin is administered to pretreat between at least 30 minutes and 360 minutes prior to the administration or release of the 2C-B HCl Form A. In some embodiments, the serotonin receptor modulator is volinanserin and the psychedelic is 2C-B HCl Form A, wherein the volinanserin is administered to pretreat between at least 60 minutes and 240 minutes prior to the administration or release of the 2C-B HCl Form A. In some embodiments, the serotonin receptor modulator is volinanserin and the psychedelic is 2C-B HCl Form A, wherein the volinanserin is administered to pretreat at least 90 minutes prior to the 2C-B HCl Form A. In some embodiments, the serotonin receptor modulator is volinanserin and the psychedelic is 2C-B HCl Form A, wherein the volinanserin is administered to pretreat at least 120 minutes prior to the 2C-B HCl Form A.

In some embodiments, the serotonin receptor modulator is volinanserin and the psychedelic is 2C-B HCl Form A, wherein the volinanserin is administered to pretreat at least 150 minutes prior to the 2C-B HCl Form A. In some embodiments, the serotonin receptor modulator is volinanserin and the psychedelic is 2C-B HCl Form A, wherein the volinanserin is administered to pretreat between about 15 minutes and about 150 minutes prior to the 2C-B HCl Form A. In some embodiments, the serotonin receptor modulator is volinanserin and the psychedelic is 2C-B HCl Form A, wherein the volinanserin is administered to pretreat at least 180 minutes prior to the 2C-B HCl Form A. In some embodiments, the serotonin receptor modulator is volinanserin and the psychedelic is 2C-B HCl Form A, wherein the volinanserin is administered to pretreat at least 210 minutes prior to the 2C-B HCl Form A. In some embodiments, the serotonin receptor modulator is volinanserin and the psychedelic is 2C-B HCl Form A, wherein the volinanserin is administered to pretreat at least 240 minutes prior to the 2C-B HCl Form A. In some embodiments, the serotonin receptor modulator is volinanserin and the psychedelic is 2C-B HCl Form A, wherein the volinanserin is administered to pretreat at least 270 minutes prior to the 2C-B HCl Form A. In some embodiments, the serotonin receptor modulator is volinanserin and the psychedelic is 2C-B HCl Form A, wherein the volinanserin is administered to pretreat at least 300 minutes prior to the 2C-B HCl Form A. In some embodiments, the serotonin receptor modulator is volinanserin and the psychedelic is 2C-B HCl Form A, wherein the volinanserin is administered to pretreat at least 330 minutes prior to the 2C-B HCl Form A. In some embodiments, the serotonin receptor modulator is volinanserin and the psychedelic is 2C-B HCl Form A, wherein the volinanserin is administered to pretreat at least 360 minutes prior to the 2C-B HCl Form A. In some preferred embodiments, the serotonin receptor modulator is volinanserin and the psychedelic is 2C-B HCl Form A, wherein volinanserin is administered to pretreat between about 60 minutes and about 180 minutes prior to the administration of the 2C-B HCl Form A.

In some embodiments, the serotonin receptor modulator is ketanserin and the psychedelic is 2C-B HCl Form A, wherein the ketanserin is administered to pretreat at least 15 minutes prior to the administration of the 2C-B HCl Form A. In some embodiments, the serotonin receptor modulator is ketanserin and the psychedelic is 2C-B HCl Form A, wherein the ketanserin is administered to pretreat between at least 30 minutes and 360 minutes prior to the administration or release of the 2C-B HCl Form A. In some embodiments, the serotonin receptor modulator is ketanserin and the psychedelic is 2C-B HCl Form A, wherein the ketanserin is administered to pretreat between at least 60 minutes and 240 minutes prior to the administration or release of the 2C-B HCl Form A. In some embodiments, the serotonin receptor modulator is ketanserin and the psychedelic is 2C-B HCl Form A, wherein the ketanserin is administered to pretreat at least 90 minutes prior to the 2C-B HCl Form A. In some embodiments, the serotonin receptor modulator is ketanserin and the psychedelic is 2C-B HCl Form A, wherein the ketanserin is administered to pretreat at least 120 minutes prior to the 2C-B HCl Form A. In some embodiments, the serotonin receptor modulator is ketanserin and the psychedelic is 2C-B HCl Form A, wherein the ketanserin is administered to pretreat at least 150 minutes prior to the 2C-B HCl Form A. In some embodiments, the serotonin receptor modulator is ketanserin and the psychedelic is 2C-B HCl Form A, wherein the ketanserin is administered to pretreat between about 15 minutes and about 150 minutes prior to the 2C-B HCl Form A. In some embodiments, the serotonin receptor modulator is ketanserin and the psychedelic is 2C-B HCl Form A, wherein the ketanserin is administered to pretreat at least 180 minutes prior to the 2C-B HCl Form A. In some embodiments, the serotonin receptor modulator is ketanserin and the psychedelic is 2C-B HCl Form A, wherein the ketanserin is administered to pretreat at least 210 minutes prior to the 2C-B HCl Form A. In some embodiments, the serotonin receptor modulator is ketanserin and the psychedelic is 2C-B HCl Form A, wherein the ketanserin is administered to pretreat at least 240 minutes prior to the 2C-B HCl Form A. In some embodiments, the serotonin receptor modulator is ketanserin and the psychedelic is 2C-B HCl Form A, wherein the ketanserin is administered to pretreat at least 270 minutes prior to the 2C-B HCl Form A. In some embodiments, the serotonin receptor modulator is ketanserin and the psychedelic is 2C-B HCl Form A, wherein the ketanserin is administered to pretreat at least 300 minutes prior to the 2C-B HCl Form A. In some embodiments, the serotonin receptor modulator is ketanserin and the psychedelic is 2C-B HCl Form A, wherein the ketanserin is administered to pretreat at least 330 minutes prior to the 2C-B HCl Form A. In some embodiments, the serotonin receptor modulator is ketanserin and the psychedelic is 2C-B HCl Form A, wherein the ketanserin is administered to pretreat at least 360 minutes prior to the 2C-B HCl Form A. In some preferred embodiments, the serotonin receptor modulator is ketanserin and the psychedelic is 2C-B HCl Form A, wherein ketanserin is administered to pretreat between about 60 minutes and about 180 minutes prior to the administration of the 2C-B HCl Form A.

In some embodiments, the serotonin receptor modulator is ritanserin and the psychedelic is 2C-B HCl Form A, wherein the ritanserin is administered to pretreat at least 15 minutes prior to the administration of the 2C-B HCl Form A. In some embodiments, the serotonin receptor modulator is ritanserin and the psychedelic is 2C-B HCl Form A, wherein the ritanserin is administered to pretreat at least 30 minutes prior to the 2C-B HCl Form A. In some embodiments, the serotonin receptor modulator is ritanserin and the psychedelic is 2C-B HCl Form A, wherein the ritanserin is administered to pretreat between at least 60 minutes and 240 minutes prior to the administration or release of the 2C-B HCl Form A. In some embodiments, the serotonin receptor modulator is ritanserin and the psychedelic is 2C-B HCl Form A, wherein the ritanserin is administered to pretreat at least 90 minutes prior to the 2C-B HCl Form A. In some embodiments, the serotonin receptor modulator is ritanserin and the psychedelic is 2C-B HCl Form A, wherein the ritanserin is administered to pretreat at least 120 minutes prior to the 2C-B HCl Form A. In some embodiments, the serotonin receptor modulator is ritanserin and the psychedelic is 2C-B HCl Form A, wherein the ritanserin is administered to pretreat at least 150 minutes prior to the 2C-B HCl Form A. In some embodiments, the serotonin receptor modulator is ritanserin and the psychedelic is 2C-B HCl Form A, wherein the ritanserin is administered to pretreat between about 15 minutes and about 150 minutes prior to the 2C-B HCl Form A. In some embodiments, the serotonin receptor modulator is ritanserin and the psychedelic is 2C-B HCl Form A, wherein the ritanserin is administered to pretreat at least 180 minutes prior to the 2C-B HCl Form A. In some embodiments, the serotonin receptor modulator is ritanserin and the psychedelic is 2C-B HCl Form A, wherein the ritanserin is administered to pretreat at least 210 minutes prior to the 2C-B HCl Form A. In some embodiments, the serotonin receptor modulator is ritanserin and the psychedelic is 2C-B HCl Form A, wherein the ritanserin is administered to pretreat at least 240 minutes prior to the 2C-B HCl Form A. In some embodiments, the serotonin receptor modulator is ritanserin and the psychedelic is 2C-B HCl Form A, wherein the ritanserin is administered to pretreat at least 270 minutes prior to the 2C-B HCl Form A. In some embodiments, the serotonin receptor modulator is ritanserin and the psychedelic is 2C-B HCl Form A, wherein the ritanserin is administered to pretreat at least 300 minutes prior to the 2C-B HCl Form A. In some embodiments, the serotonin receptor modulator is ritanserin and the psychedelic is 2C-B HCl Form A, wherein the ritanserin is administered to pretreat at least 330 minutes prior to the 2C-B HCl Form A. In some embodiments, the serotonin receptor modulator is ritanserin and the psychedelic is 2C-B HCl Form A, wherein the ritanserin is administered to pretreat at least 360 minutes prior to the 2C-B HCl Form A. In some preferred embodiments, the serotonin receptor modulator is ritanserin and the psychedelic is 2C-B HCl Form A, wherein ritanserin is administered to pretreat between about 60 minutes and about 180 minutes prior to the administration of the 2C-B HCl Form A.

In some embodiments, the serotonin receptor modulator is pimavanserin and the psychedelic is 2C-B HCl Form A, wherein the pimavanserin is administered to pretreat at least 15 minutes prior to the administration of the 2C-B HCl Form A. In some embodiments, the serotonin receptor modulator is pimavanserin and the psychedelic is 2C-B HCl Form A, wherein the pimavanserin is administered to pretreat at least 30 minutes prior to the 2C-B HCl Form A. In some embodiments, the serotonin receptor modulator is pimavanserin and the psychedelic is 2C-B HCl Form A, wherein the pimavanserin is administered to pretreat between at least 60 minutes and 240 minutes prior to the administration or release of the 2C-B HCl Form A. In some embodiments, the serotonin receptor modulator is pimavanserin and the psychedelic is 2C-B HCl Form A, wherein the pimavanserin is administered to pretreat at least 90 minutes prior to the 2C-B HCl Form A. In some embodiments, the serotonin receptor modulator is pimavanserin and the psychedelic is 2C-B HCl Form A, wherein the pimavanserin is administered to pretreat at least 120 minutes prior to the 2C-B HCl Form A. In some embodiments, the serotonin receptor modulator is pimavanserin and the psychedelic is 2C-B HCl Form A, wherein the pimavanserin is administered to pretreat at least 150 minutes prior to the 2C-B HCl Form A. In some embodiments, the serotonin receptor modulator is pimavanserin and the psychedelic is 2C-B HCl Form A, wherein the pimavanserin is administered to pretreat between about 15 minutes and about 150 minutes prior to the 2C-B HCl Form A. In some embodiments, the serotonin receptor modulator is pimavanserin and the psychedelic is 2C-B HCl Form A, wherein the pimavanserin is administered to pretreat at least 180 minutes prior to the 2C-B HCl Form A. In some embodiments, the serotonin receptor modulator is pimavanserin and the psychedelic is 2C-B HCl Form A, wherein the pimavanserin is administered to pretreat at least 210 minutes prior to the 2C-B HCl Form A. In some embodiments, the serotonin receptor modulator is pimavanserin and the psychedelic is 2C-B HCl Form A, wherein the pimavanserin is administered to pretreat at least 240 minutes prior to the 2C-B HCl Form A. In some embodiments, the serotonin receptor modulator is pimavanserin and the psychedelic is 2C-B HCl Form A, wherein the pimavanserin is administered to pretreat at least 270 minutes prior to the 2C-B HCl Form A. In some embodiments, the serotonin receptor modulator is pimavanserin and the psychedelic is 2C-B HCl Form A, wherein the pimavanserin is administered to pretreat at least 300 minutes prior to the 2C-B HCl Form A. In some embodiments, the serotonin receptor modulator is pimavanserin and the psychedelic is 2C-B HCl Form A, wherein the pimavanserin is administered to pretreat at least 330 minutes prior to the 2C-B HCl Form A. In some embodiments, the serotonin receptor modulator is pimavanserin and the psychedelic is 2C-B HCl Form A, wherein the pimavanserin is administered to pretreat at least 360 minutes prior to the 2C-B HCl Form A. In some preferred embodiments, the serotonin receptor modulator is pimavanserin and the psychedelic is 2C-B HCl Form A, wherein pimavanserin is administered to pretreat between about 60 minutes and about 180 minutes prior to the administration of the 2C-B HCl Form A.

In some embodiments, the serotonin receptor modulator is nelotanserin and the psychedelic is 2C-B HCl Form A, wherein the nelotanserin is administered to pretreat at least 15 minutes prior to the administration of the 2C-B HCl Form A. In some embodiments, the serotonin receptor modulator is nelotanserin and the psychedelic is 2C-B HCl Form A, wherein the nelotanserin is administered to pretreat at least 30 minutes prior to the 2C-B HCl Form A. In some embodiments, the serotonin receptor modulator is nelotanserin and the psychedelic is 2C-B HCl Form A, wherein the nelotanserin is administered to pretreat between at least 60 minutes and 240 minutes prior to the administration or release of the 2C-B HCl Form A. In some embodiments, the serotonin receptor modulator is nelotanserin and the psychedelic is 2C-B HCl Form A, wherein the nelotanserin is administered to pretreat at least 90 minutes prior to the 2C-B HCl Form A. In some embodiments, the serotonin receptor modulator is nelotanserin and the psychedelic is 2C-B HCl Form A, wherein the nelotanserin is administered to pretreat at least 120 minutes prior to the 2C-B HCl Form A.

In some embodiments, the serotonin receptor modulator is nelotanserin and the psychedelic is 2C-B HCl Form A, wherein the nelotanserin is administered to pretreat at least 150 minutes prior to the 2C-B HCl Form A. In some embodiments, the serotonin receptor modulator is nelotanserin and the psychedelic is 2C-B HCl Form A, wherein the nelotanserin is administered to pretreat between about 15 minutes and about 150 minutes prior to the 2C-B HCl Form A. In some embodiments, the serotonin receptor modulator is nelotanserin and the psychedelic is 2C-B HCl Form A, wherein the nelotanserin is administered to pretreat at least 180 minutes prior to the 2C-B HCl Form A. In some embodiments, the serotonin receptor modulator is nelotanserin and the psychedelic is 2C-B HCl Form A, wherein the nelotanserin is administered to pretreat at least 210 minutes prior to the 2C-B HCl Form A. In some embodiments, the serotonin receptor modulator is nelotanserin and the psychedelic is 2C-B HCl Form A, wherein the nelotanserin is administered to pretreat at least 240 minutes prior to the 2C-B HCl Form A. In some embodiments, the serotonin receptor modulator is nelotanserin and the psychedelic is 2C-B HCl Form A, wherein the nelotanserin is administered to pretreat at least 270 minutes prior to the 2C-B HCl Form A.

In some embodiments, the serotonin receptor modulator is nelotanserin and the psychedelic is 2C-B HCl Form A, wherein the nelotanserin is administered to pretreat at least 300 minutes prior to the 2C-B HCl Form A. In some embodiments, the serotonin receptor modulator is nelotanserin and the psychedelic is 2C-B HCl Form A, wherein the nelotanserin is administered to pretreat at least 330 minutes prior to the 2C-B HCl Form A. In some embodiments, the serotonin receptor modulator is nelotanserin and the psychedelic is 2C-B HCl Form A, wherein the nelotanserin is administered to pretreat at least 360 minutes prior to the 2C-B HCl Form A. In some preferred embodiments, the serotonin receptor modulator is nelotanserin and the psychedelic is 2C-B HCl Form A, wherein nelotanserin is administered to pretreat between about 60 minutes and about 180 minutes prior to the administration of the 2C-B HCl Form A.

In some embodiments, the serotonin receptor modulator is pruvanserin and the psychedelic is 2C-B HCl Form A, wherein the pruvanserin is administered to pretreat at least 15 minutes prior to the administration of the 2C-B HCl Form A. In some embodiments, the serotonin receptor modulator is pruvanserin and the psychedelic is 2C-B HCl Form A, wherein the pruvanserin is administered to pretreat at least 30 minutes prior to the 2C-B HCl Form A. In some embodiments, the serotonin receptor modulator is pruvanserin and the psychedelic is 2C-B HCl Form A, wherein the pruvanserin is administered to pretreat between at least 60 minutes and 240 minutes prior to the administration or release of the 2C-B HCl Form A. In some embodiments, the serotonin receptor modulator is pruvanserin and the psychedelic is 2C-B HCl Form A, wherein the pruvanserin is administered to pretreat at least 90 minutes prior to the 2C-B HCl Form A. In some embodiments, the serotonin receptor modulator is pruvanserin and the psychedelic is 2C-B HCl Form A, wherein the pruvanserin is administered to pretreat at least 120 minutes prior to the 2C-B HCl Form A. In some embodiments, the serotonin receptor modulator is pruvanserin and the psychedelic is 2C-B HCl Form A, wherein the pruvanserin is administered to pretreat at least 150 minutes prior to the 2C-B HCl Form A. In some embodiments, the serotonin receptor modulator is pruvanserin and the psychedelic is 2C-B HCl Form A, wherein the pruvanserin is administered to pretreat between about 15 minutes and about 150 minutes prior to the 2C-B HCl Form A. In some embodiments, the serotonin receptor modulator is pruvanserin and the psychedelic is 2C-B HCl Form A, wherein the pruvanserin is administered to pretreat at least 180 minutes prior to the 2C-B HCl Form A. In some embodiments, the serotonin receptor modulator is pruvanserin and the psychedelic is 2C-B HCl Form A, wherein the pruvanserin is administered to pretreat at least 210 minutes prior to the 2C-B HCl Form A.

In some embodiments, the serotonin receptor modulator is pruvanserin and the psychedelic is 2C-B HCl Form A, wherein the pruvanserin is administered to pretreat at least 240 minutes prior to the 2C-B HCl Form A. In some embodiments, the serotonin receptor modulator is pruvanserin and the psychedelic is 2C-B HCl Form A, wherein the pruvanserin is administered to pretreat at least 270 minutes prior to the 2C-B HCl Form A. In some embodiments, the serotonin receptor modulator is pruvanserin and the psychedelic is 2C-B HCl Form A, wherein the pruvanserin is administered to pretreat at least 300 minutes prior to the 2C-B HCl Form A. In some embodiments, the serotonin receptor modulator is pruvanserin and the psychedelic is 2C-B HCl Form A, wherein the pruvanserin is administered to pretreat at least 330 minutes prior to the 2C-B HCl Form A. In some embodiments, the serotonin receptor modulator is pruvanserin and the psychedelic is 2C-B HCl Form A, wherein the pruvanserin is administered to pretreat at least 360 minutes prior to the 2C-B HCl Form A. In some preferred embodiments, the serotonin receptor modulator is pruvanserin and the psychedelic is 2C-B HCl Form A, wherein pruvanserin is administered to pretreat between about 60 minutes and about 180 minutes prior to the administration of the 2C-B HCl Form A.

In some embodiments, the serotonin receptor modulator is flibanserin and the psychedelic is 2C-B HCl Form A, wherein the flibanserin is administered to pretreat at least 15 minutes prior to the administration of the 2C-B HCl Form A. In some embodiments, the serotonin receptor modulator is flibanserin and the psychedelic is 2C-B HCl Form A, wherein the flibanserin is administered to pretreat at least 30 minutes prior to the 2C-B HCl Form A. In some embodiments, the serotonin receptor modulator is flibanserin and the psychedelic is 2C-B HCl Form A, wherein the flibanserin is administered to pretreat between at least 60 minutes and 240 minutes prior to the administration or release of the 2C-B HCl Form A. In some embodiments, the serotonin receptor modulator is flibanserin and the psychedelic is 2C-B HCl Form A, wherein the flibanserin is administered to pretreat at least 90 minutes prior to the 2C-B HCl Form A. In some embodiments, the serotonin receptor modulator is flibanserin and the psychedelic is 2C-B HCl Form A, wherein the flibanserin is administered to pretreat at least 120 minutes prior to the 2C-B HCl Form A. In some embodiments, the serotonin receptor modulator is flibanserin and the psychedelic is 2C-B HCl Form A, wherein the flibanserin is administered to pretreat at least 150 minutes prior to the 2C-B HCl Form A. In some embodiments, the serotonin receptor modulator is flibanserin and the psychedelic is 2C-B HCl Form A, wherein the flibanserin is administered to pretreat between about 15 minutes and about 150 minutes prior to the 2C-B HCl Form A. In some embodiments, the serotonin receptor modulator is flibanserin and the psychedelic is 2C-B HCl Form A, wherein the flibanserin is administered to pretreat at least 180 minutes prior to the 2C-B HCl Form A. In some embodiments, the serotonin receptor modulator is flibanserin and the psychedelic is 2C-B HCl Form A, wherein the flibanserin is administered to pretreat at least 210 minutes prior to the 2C-B HCl Form A.

In some embodiments, the serotonin receptor modulator is flibanserin and the psychedelic is 2C-B HCl Form A, wherein the flibanserin is administered to pretreat at least 240 minutes prior to the 2C-B HCl Form A. In some embodiments, the serotonin receptor modulator is flibanserin and the psychedelic is 2C-B HCl Form A, wherein the flibanserin is administered to pretreat at least 270 minutes prior to the 2C-B HCl Form A. In some embodiments, the serotonin receptor modulator is flibanserin and the psychedelic is 2C-B HCl Form A, wherein the flibanserin is administered to pretreat at least 300 minutes prior to the 2C-B HCl Form A. In some embodiments, the serotonin receptor modulator is flibanserin and the psychedelic is 2C-B HCl Form A, wherein the flibanserin is administered to pretreat at least 330 minutes prior to the 2C-B HCl Form A. In some embodiments, the serotonin receptor modulator is flibanserin and the psychedelic is 2C-B HCl Form A, wherein the flibanserin is administered to pretreat at least 360 minutes prior to the 2C-B HCl Form A. In some preferred embodiments, the serotonin receptor modulator is flibanserin and the psychedelic is 2C-B HCl Form A, wherein flibanserin is administered to pretreat between about 60 minutes and about 180 minutes prior to the administration of the 2C-B HCl Form A.

In some embodiments, the serotonin receptor modulator is olanzapine and the psychedelic is 2C-B HCl Form A, wherein the olanzapine is administered to pretreat at least 15 minutes prior to the administration of the 2C-B HCl Form A. In some embodiments, the serotonin receptor modulator is olanzapine and the psychedelic is 2C-B HCl Form A, wherein the olanzapine is administered to pretreat at least 30 minutes prior to the 2C-B HCl Form A. In some embodiments, the serotonin receptor modulator is olanzapine and the psychedelic is 2C-B HCl Form A, wherein the olanzapine is administered to pretreat between at least 60 minutes and 240 minutes prior to the administration or release of the 2C-B HCl Form A. In some embodiments, the serotonin receptor modulator is olanzapine and the psychedelic is 2C-B HCl Form A, wherein the olanzapine is administered to pretreat at least 90 minutes prior to the 2C-B HCl Form A. In some embodiments, the serotonin receptor modulator is olanzapine and the psychedelic is 2C-B HCl Form A, wherein the olanzapine is administered to pretreat at least 120 minutes prior to the 2C-B HCl Form A. In some embodiments, the serotonin receptor modulator is olanzapine and the psychedelic is 2C-B HCl Form A, wherein the olanzapine is administered to pretreat at least 150 minutes prior to the 2C-B HCl Form A. In some embodiments, the serotonin receptor modulator is olanzapine and the psychedelic is 2C-B HCl Form A, wherein the olanzapine is administered to pretreat between about 15 minutes and about 150 minutes prior to the 2C-B HCl Form A. In some embodiments, the serotonin receptor modulator is olanzapine and the psychedelic is 2C-B HCl Form A, wherein the olanzapine is administered to pretreat at least 180 minutes prior to the 2C-B HCl Form A. In some embodiments, the serotonin receptor modulator is olanzapine and the psychedelic is 2C-B HCl Form A, wherein the olanzapine is administered to pretreat at least 210 minutes prior to the 2C-B HCl Form A.

In some embodiments, the serotonin receptor modulator is olanzapine and the psychedelic is 2C-B HCl Form A, wherein the olanzapine is administered to pretreat at least 240 minutes prior to the 2C-B HCl Form A. In some embodiments, the serotonin receptor modulator is olanzapine and the psychedelic is 2C-B HCl Form A, wherein the olanzapine is administered to pretreat at least 270 minutes prior to the 2C-B HCl Form A. In some embodiments, the serotonin receptor modulator is olanzapine and the psychedelic is 2C-B HCl Form A, wherein the olanzapine is administered to pretreat at least 300 minutes prior to the 2C-B HCl Form A. In some embodiments, the serotonin receptor modulator is olanzapine and the psychedelic is 2C-B HCl Form A, wherein the olanzapine is administered to pretreat at least 330 minutes prior to the 2C-B HCl Form A. In some embodiments, the serotonin receptor modulator is olanzapine and the psychedelic is 2C-B HCl Form A, wherein the olanzapine is administered to pretreat at least 360 minutes prior to the 2C-B HCl Form A. In some preferred embodiments, the serotonin receptor modulator is olanzapine and the psychedelic is 2C-B HCl Form A, wherein olanzapine is administered to pretreat between about 60 minutes and about 180 minutes prior to the administration of the 2C-B HCl Form A.

In some embodiments, the serotonin receptor modulator is risperidone and the psychedelic is 2C-B HCl Form A, wherein the risperidone is administered to pretreat at least 15 minutes prior to the administration of the 2C-B HCl Form A. In some embodiments, the serotonin receptor modulator is risperidone and the psychedelic is 2C-B HCl Form A, wherein the risperidone is administered to pretreat at least 30 minutes prior to the 2C-B HCl Form A. In some embodiments, the serotonin receptor modulator is risperidone and the psychedelic is 2C-B HCl Form A, wherein the risperidone is administered to pretreat between at least 60 minutes and 240 minutes prior to the administration or release of the 2C-B HCl Form A. In some embodiments, the serotonin receptor modulator is risperidone and the psychedelic is 2C-B HCl Form A, wherein the risperidone is administered to pretreat at least 90 minutes prior to the 2C-B HCl Form A. In some embodiments, the serotonin receptor modulator is risperidone and the psychedelic is 2C-B HCl Form A, wherein the risperidone is administered to pretreat at least 120 minutes prior to the 2C-B HCl Form A. In some embodiments, the serotonin receptor modulator is risperidone and the psychedelic is 2C-B HCl Form A, wherein the risperidone is administered to pretreat at least 150 minutes prior to the 2C-B HCl Form A. In some embodiments, the serotonin receptor modulator is risperidone and the psychedelic is 2C-B HCl Form A, wherein the risperidone is administered to pretreat between about 15 minutes and about 150 minutes prior to the 2C-B HCl Form A. In some embodiments, the serotonin receptor modulator is risperidone and the psychedelic is 2C-B HCl Form A, wherein the risperidone is administered to pretreat at least 180 minutes prior to the 2C-B HCl Form A. In some embodiments, the serotonin receptor modulator is risperidone and the psychedelic is 2C-B HCl Form A, wherein the risperidone is administered to pretreat at least 210 minutes prior to the 2C-B HCl Form A.

In some embodiments, the serotonin receptor modulator is risperidone and the psychedelic is 2C-B HCl Form A, wherein the risperidone is administered to pretreat at least 240 minutes prior to the 2C-B HCl Form A. In some embodiments, the serotonin receptor modulator is risperidone and the psychedelic is 2C-B HCl Form A, wherein the risperidone is administered to pretreat at least 270 minutes prior to the 2C-B HCl Form A. In some embodiments, the serotonin receptor modulator is risperidone and the psychedelic is 2C-B HCl Form A, wherein the risperidone is administered to pretreat at least 300 minutes prior to the 2C-B HCl Form A. In some embodiments, the serotonin receptor modulator is risperidone and the psychedelic is 2C-B HCl Form A, wherein the risperidone is administered to pretreat at least 330 minutes prior to the 2C-B HCl Form A. In some embodiments, the serotonin receptor modulator is risperidone and the psychedelic is 2C-B HCl Form A, wherein the risperidone is administered to pretreat at least 360 minutes prior to the 2C-B HCl Form A. In some preferred embodiments, the serotonin receptor modulator is risperidone and the psychedelic is 2C-B HCl Form A, wherein risperidone is administered to pretreat between about 60 minutes and about 180 minutes prior to the administration of the 2C-B HCl Form A.

In some embodiments, the serotonin receptor modulator is quetiapine and the psychedelic is 2C-B HCl Form A, wherein the quetiapine is administered to pretreat at least 15 minutes prior to the administration of the 2C-B HCl Form A. In some embodiments, the serotonin receptor modulator is quetiapine and the psychedelic is 2C-B HCl Form A, wherein the quetiapine is administered to pretreat at least 30 minutes prior to the 2C-B HCl Form A. In some embodiments, the serotonin receptor modulator is quetiapine and the psychedelic is 2C-B HCl Form A, wherein the quetiapine is administered to pretreat between at least 60 minutes and 240 minutes prior to the administration or release of the 2C-B HCl Form A. In some embodiments, the serotonin receptor modulator is quetiapine and the psychedelic is 2C-B HCl Form A, wherein the quetiapine is administered to pretreat at least 90 minutes prior to the 2C-B HCl Form A. In some embodiments, the serotonin receptor modulator is quetiapine and the psychedelic is 2C-B HCl Form A, wherein the quetiapine is administered to pretreat at least 120 minutes prior to the 2C-B HCl Form A. In some embodiments, the serotonin receptor modulator is quetiapine and the psychedelic is 2C-B HCl Form A, wherein the quetiapine is administered to pretreat at least 150 minutes prior to the 2C-B HCl Form A. In some embodiments, the serotonin receptor modulator is quetiapine and the psychedelic is 2C-B HCl Form A, wherein the quetiapine is administered to pretreat between about 15 minutes and about 150 minutes prior to the 2C-B HCl Form A. In some embodiments, the serotonin receptor modulator is quetiapine and the psychedelic is 2C-B HCl Form A, wherein the quetiapine is administered to pretreat at least 180 minutes prior to the 2C-B HCl Form A. In some embodiments, the serotonin receptor modulator is quetiapine and the psychedelic is 2C-B HCl Form A, wherein the quetiapine is administered to pretreat at least 210 minutes prior to the 2C-B HCl Form A.

In some embodiments, the serotonin receptor modulator is quetiapine and the psychedelic is 2C-B HCl Form A, wherein the quetiapine is administered to pretreat at least 240 minutes prior to the 2C-B HCl Form A. In some embodiments, the serotonin receptor modulator is quetiapine and the psychedelic is 2C-B HCl Form A, wherein the quetiapine is administered to pretreat at least 270 minutes prior to the 2C-B HCl Form A. In some embodiments, the serotonin receptor modulator is quetiapine and the psychedelic is 2C-B HCl Form A, wherein the quetiapine is administered to pretreat at least 300 minutes prior to the 2C-B HCl Form A. In some embodiments, the serotonin receptor modulator is quetiapine and the psychedelic is 2C-B HCl Form A, wherein the quetiapine is administered to pretreat at least 330 minutes prior to the 2C-B HCl Form A. In some embodiments, the serotonin receptor modulator is quetiapine and the psychedelic is 2C-B HCl Form A, wherein the quetiapine is administered to pretreat at least 360 minutes prior to the 2C-B HCl Form A. In some preferred embodiments, the serotonin receptor modulator is quetiapine and the psychedelic is 2C-B HCl Form A, wherein quetiapine is administered to pretreat between about 60 minutes and about 180 minutes prior to the administration of the 2C-B HCl Form A.

In some embodiments, the serotonin receptor modulator is eplivanserin and the psychedelic is 2C-B HCl Form C, wherein the eplivanserin is administered to pretreat at least 15 minutes prior to the administration of the 2C-B HCl Form C. In some embodiments, the serotonin receptor modulator is eplivanserin and the psychedelic is 2C-B HCl Form C, wherein the eplivanserin is administered to pretreat between at least 30 minutes prior and 360 minutes prior to the release or administration of the 2C-B HCl Form C. In some embodiments, the serotonin receptor modulator is eplivanserin and the psychedelic is 2C-B HCl Form C, wherein the eplivanserin is administered to pretreat between at least 60 minutes prior and 360 minutes prior to the release or administration of the 2C-B HCl Form C. In some embodiments, the serotonin receptor modulator is eplivanserin and the psychedelic is 2C-B HCl Form C, wherein the eplivanserin is administered to pretreat between at least 90 minutes and 240 minutes prior to the 2C-B HCl Form C. In some embodiments, the serotonin receptor modulator is eplivanserin and the psychedelic is 2C-B HCl Form C, wherein the eplivanserin is administered to pretreat at least 120 minutes prior to the 2C-B HCl Form C.

In some embodiments, the serotonin receptor modulator is eplivanserin and the psychedelic is 2C-B HCl Form C, wherein the eplivanserin is administered to pretreat at least 150 minutes prior to the 2C-B HCl Form C. In some embodiments, the serotonin receptor modulator is eplivanserin and the psychedelic is 2C-B HCl Form C, wherein the eplivanserin is administered to pretreat between about 15 minutes and about 150 minutes prior to the 2C-B HCl Form C. In some embodiments, the serotonin receptor modulator is eplivanserin and the psychedelic is 2C-B HCl Form C, wherein the eplivanserin is administered to pretreat at least 180 minutes prior to the 2C-B HCl Form C. In some embodiments, the serotonin receptor modulator is eplivanserin and the psychedelic is 2C-B HCl Form C, wherein the eplivanserin is administered to pretreat at least 210 minutes prior to the 2C-B HCl Form C. In some embodiments, the serotonin receptor modulator is eplivanserin and the psychedelic is 2C-B HCl Form C, wherein the eplivanserin is administered to pretreat at least 240 minutes prior to the 2C-B HCl Form C.

In some embodiments, the serotonin receptor modulator is eplivanserin and the psychedelic is 2C-B HCl Form C, wherein the eplivanserin is administered to pretreat at least 270 minutes prior to 2C-B HCl Form C. In some embodiments, the serotonin receptor modulator is eplivanserin and the psychedelic is 2C-B HCl Form C, wherein the eplivanserin is administered to pretreat at least 300 minutes prior to the 2C-B HCl Form C. In some embodiments, the serotonin receptor modulator is eplivanserin and the psychedelic is 2C-B HCl Form C, wherein the eplivanserin is administered to pretreat at least 330 minutes prior to the 2C-B HCl Form C. In some embodiments, the serotonin receptor modulator is eplivanserin and the psychedelic is 2C-B HCl Form C, wherein the eplivanserin is administered to pretreat at least 360 minutes prior to the 2C-B HCl Form C.

In some preferred embodiments, the serotonin receptor modulator is eplivanserin and the psychedelic is 2C-B HCl Form C, wherein eplivanserin is administered to pretreat between about 60 minutes and about 180 minutes prior to the administration of the 2C-B HCl Form C. In some embodiments, the serotonin receptor modulator is volinanserin and the psychedelic is 2C-B HCl Form C, wherein the volinanserin is administered to pretreat a subject between at least 15 minutes and 360 minutes prior to the administration or release of the 2C-B HCl Form C. In some embodiments, the serotonin receptor modulator is volinanserin and the psychedelic is 2C-B HCl Form C, wherein the volinanserin is administered to pretreat between at least 30 minutes and 360 minutes prior to the administration or release of the 2C-B HCl Form C. In some embodiments, the serotonin receptor modulator is volinanserin and the psychedelic is 2C-B HCl Form C, wherein the volinanserin is administered to pretreat between at least 60 minutes and 240 minutes prior to the administration or release of the 2C-B HCl Form C. In some embodiments, the serotonin receptor modulator is volinanserin and the psychedelic is 2C-B HCl Form C, wherein the volinanserin is administered to pretreat at least 90 minutes prior to the 2C-B HCl Form C. In some embodiments, the serotonin receptor modulator is volinanserin and the psychedelic is 2C-B HCl Form C, wherein the volinanserin is administered to pretreat at least 120 minutes prior to the 2C-B HCl Form C.

In some embodiments, the serotonin receptor modulator is volinanserin and the psychedelic is 2C-B HCl Form C, wherein the volinanserin is administered to pretreat at least 150 minutes prior to the 2C-B HCl Form C. In some embodiments, the serotonin receptor modulator is volinanserin and the psychedelic is 2C-B HCl Form C, wherein the volinanserin is administered to pretreat between about 15 minutes and about 150 minutes prior to the 2C-B HCl Form C. In some embodiments, the serotonin receptor modulator is volinanserin and the psychedelic is 2C-B HCl Form C, wherein the volinanserin is administered to pretreat at least 180 minutes prior to the 2C-B HCl Form C. In some embodiments, the serotonin receptor modulator is volinanserin and the psychedelic is 2C-B HCl Form C, wherein the volinanserin is administered to pretreat at least 210 minutes prior to the 2C-B HCl Form C. In some embodiments, the serotonin receptor modulator is volinanserin and the psychedelic is 2C-B HCl Form C, wherein the volinanserin is administered to pretreat at least 240 minutes prior to the 2C-B HCl Form C. In some embodiments, the serotonin receptor modulator is volinanserin and the psychedelic is 2C-B HCl Form C, wherein the volinanserin is administered to pretreat at least 270 minutes prior to the 2C-B HCl Form C. In some embodiments, the serotonin receptor modulator is volinanserin and the psychedelic is 2C-B HCl Form C, wherein the volinanserin is administered to pretreat at least 300 minutes prior to the 2C-B HCl Form C. In some embodiments, the serotonin receptor modulator is volinanserin and the psychedelic is 2C-B HCl Form C, wherein the volinanserin is administered to pretreat at least 330 minutes prior to the 2C-B HCl Form C. In some embodiments, the serotonin receptor modulator is volinanserin and the psychedelic is 2C-B HCl Form C, wherein the volinanserin is administered to pretreat at least 360 minutes prior to the 2C-B HCl Form C. In some preferred embodiments, the serotonin receptor modulator is volinanserin and the psychedelic is 2C-B HCl Form C, wherein volinanserin is administered to pretreat between about 60 minutes and about 180 minutes prior to the administration of the 2C-B HCl Form C.

In some embodiments, the serotonin receptor modulator is ketanserin and the psychedelic is 2C-B HCl Form C, wherein the ketanserin is administered to pretreat at least 15 minutes prior to the administration of the 2C-B HCl Form C. In some embodiments, the serotonin receptor modulator is ketanserin and the psychedelic is 2C-B HCl Form C, wherein the ketanserin is administered to pretreat between at least 30 minutes and 360 minutes prior to the administration or release of the 2C-B HCl Form C. In some embodiments, the serotonin receptor modulator is ketanserin and the psychedelic is 2C-B HCl Form C, wherein the ketanserin is administered to pretreat between at least 60 minutes and 240 minutes prior to the administration or release of the 2C-B HCl Form C. In some embodiments, the serotonin receptor modulator is ketanserin and the psychedelic is 2C-B HCl Form C, wherein the ketanserin is administered to pretreat at least 90 minutes prior to the 2C-B HCl Form C. In some embodiments, the serotonin receptor modulator is ketanserin and the psychedelic is 2C-B HCl Form C, wherein the ketanserin is administered to pretreat at least 120 minutes prior to the 2C-B HCl Form C. In some embodiments, the serotonin receptor modulator is ketanserin and the psychedelic is 2C-B HCl Form C, wherein the ketanserin is administered to pretreat at least 150 minutes prior to the 2C-B HCl Form C. In some embodiments, the serotonin receptor modulator is ketanserin and the psychedelic is 2C-B HCl Form C, wherein the ketanserin is administered to pretreat between about 15 minutes and about 150 minutes prior to the 2C-B HCl Form C. In some embodiments, the serotonin receptor modulator is ketanserin and the psychedelic is 2C-B HCl Form C, wherein the ketanserin is administered to pretreat at least 180 minutes prior to the 2C-B HCl Form C. In some embodiments, the serotonin receptor modulator is ketanserin and the psychedelic is 2C-B HCl Form C, wherein the ketanserin is administered to pretreat at least 210 minutes prior to the 2C-B HCl Form C. In some embodiments, the serotonin receptor modulator is ketanserin and the psychedelic is 2C-B HCl Form C, wherein the ketanserin is administered to pretreat at least 240 minutes prior to the 2C-B HCl Form C. In some embodiments, the serotonin receptor modulator is ketanserin and the psychedelic is 2C-B HCl Form C, wherein the ketanserin is administered to pretreat at least 270 minutes prior to the 2C-B HCl Form C. In some embodiments, the serotonin receptor modulator is ketanserin and the psychedelic is 2C-B HCl Form C, wherein the ketanserin is administered to pretreat at least 300 minutes prior to the 2C-B HCl Form C. In some embodiments, the serotonin receptor modulator is ketanserin and the psychedelic is 2C-B HCl Form C, wherein the ketanserin is administered to pretreat at least 330 minutes prior to the 2C-B HCl Form C. In some embodiments, the serotonin receptor modulator is ketanserin and the psychedelic is 2C-B HCl Form C, wherein the ketanserin is administered to pretreat at least 360 minutes prior to the 2C-B HCl Form C. In some preferred embodiments, the serotonin receptor modulator is ketanserin and the psychedelic is 2C-B HCl Form C, wherein ketanserin is administered to pretreat between about 60 minutes and about 180 minutes prior to the administration of the 2C-B HCl Form C.

In some embodiments, the serotonin receptor modulator is ritanserin and the psychedelic is 2C-B HCl Form C, wherein the ritanserin is administered to pretreat at least 15 minutes prior to the administration of the 2C-B HCl Form C. In some embodiments, the serotonin receptor modulator is ritanserin and the psychedelic is 2C-B HCl Form C, wherein the ritanserin is administered to pretreat at least 30 minutes prior to the 2C-B HCl Form C. In some embodiments, the serotonin receptor modulator is ritanserin and the psychedelic is 2C-B HCl Form C, wherein the ritanserin is administered to pretreat between at least 60 minutes and 240 minutes prior to the administration or release of the 2C-B HCl Form C. In some embodiments, the serotonin receptor modulator is ritanserin and the psychedelic is 2C-B HCl Form C, wherein the ritanserin is administered to pretreat at least 90 minutes prior to the 2C-B HCl Form C. In some embodiments, the serotonin receptor modulator is ritanserin and the psychedelic is 2C-B HCl Form C, wherein the ritanserin is administered to pretreat at least 120 minutes prior to the 2C-B HCl Form C. In some embodiments, the serotonin receptor modulator is ritanserin and the psychedelic is 2C-B HCl Form C, wherein the ritanserin is administered to pretreat at least 150 minutes prior to the 2C-B HCl Form C. In some embodiments, the serotonin receptor modulator is ritanserin and the psychedelic is 2C-B HCl Form C, wherein the ritanserin is administered to pretreat between about 15 minutes and about 150 minutes prior to the 2C-B HCl Form C. In some embodiments, the serotonin receptor modulator is ritanserin and the psychedelic is 2C-B HCl Form C, wherein the ritanserin is administered to pretreat at least 180 minutes prior to the 2C-B HCl Form C. In some embodiments, the serotonin receptor modulator is ritanserin and the psychedelic is 2C-B HCl Form C, wherein the ritanserin is administered to pretreat at least 210 minutes prior to the 2C-B HCl Form C. In some embodiments, the serotonin receptor modulator is ritanserin and the psychedelic is 2C-B HCl Form C, wherein the ritanserin is administered to pretreat at least 240 minutes prior to the 2C-B HCl Form C. In some embodiments, the serotonin receptor modulator is ritanserin and the psychedelic is 2C-B HCl Form C, wherein the ritanserin is administered to pretreat at least 270 minutes prior to the 2C-B HCl Form C. In some embodiments, the serotonin receptor modulator is ritanserin and the psychedelic is 2C-B HCl Form C, wherein the ritanserin is administered to pretreat at least 300 minutes prior to the 2C-B HCl Form C. In some embodiments, the serotonin receptor modulator is ritanserin and the psychedelic is 2C-B HCl Form C, wherein the ritanserin is administered to pretreat at least 330 minutes prior to the 2C-B HCl Form C. In some embodiments, the serotonin receptor modulator is ritanserin and the psychedelic is 2C-B HCl Form C, wherein the ritanserin is administered to pretreat at least 360 minutes prior to the 2C-B HCl Form C. In some preferred embodiments, the serotonin receptor modulator is ritanserin and the psychedelic is 2C-B HCl Form C, wherein ritanserin is administered to pretreat between about 60 minutes and about 180 minutes prior to the administration of the 2C-B HCl Form C.

In some embodiments, the serotonin receptor modulator is pimavanserin and the psychedelic is 2C-B HCl Form C, wherein the pimavanserin is administered to pretreat at least 15 minutes prior to the administration of the 2C-B HCl Form C. In some embodiments, the serotonin receptor modulator is pimavanserin and the psychedelic is 2C-B HCl Form C, wherein the pimavanserin is administered to pretreat at least 30 minutes prior to the 2C-B HCl Form C. In some embodiments, the serotonin receptor modulator is pimavanserin and the psychedelic is 2C-B HCl Form C, wherein the pimavanserin is administered to pretreat between at least 60 minutes and 240 minutes prior to the administration or release of the 2C-B HCl Form C. In some embodiments, the serotonin receptor modulator is pimavanserin and the psychedelic is 2C-B HCl Form C, wherein the pimavanserin is administered to pretreat at least 90 minutes prior to the 2C-B HCl Form C. In some embodiments, the serotonin receptor modulator is pimavanserin and the psychedelic is 2C-B HCl Form C, wherein the pimavanserin is administered to pretreat at least 120 minutes prior to the 2C-B HCl Form C. In some embodiments, the serotonin receptor modulator is pimavanserin and the psychedelic is 2C-B HCl Form C, wherein the pimavanserin is administered to pretreat at least 150 minutes prior to the 2C-B HCl Form C. In some embodiments, the serotonin receptor modulator is pimavanserin and the psychedelic is 2C-B HCl Form C, wherein the pimavanserin is administered to pretreat between about 15 minutes and about 150 minutes prior to the 2C-B HCl Form C. In some embodiments, the serotonin receptor modulator is pimavanserin and the psychedelic is 2C-B HCl Form C, wherein the pimavanserin is administered to pretreat at least 180 minutes prior to the 2C-B HCl Form C. In some embodiments, the serotonin receptor modulator is pimavanserin and the psychedelic is 2C-B HCl Form C, wherein the pimavanserin is administered to pretreat at least 210 minutes prior to the 2C-B HCl Form C. In some embodiments, the serotonin receptor modulator is pimavanserin and the psychedelic is 2C-B HCl Form C, wherein the pimavanserin is administered to pretreat at least 240 minutes prior to the 2C-B HCl Form C. In some embodiments, the serotonin receptor modulator is pimavanserin and the psychedelic is 2C-B HCl Form C, wherein the pimavanserin is administered to pretreat at least 270 minutes prior to the 2C-B HCl Form C. In some embodiments, the serotonin receptor modulator is pimavanserin and the psychedelic is 2C-B HCl Form C, wherein the pimavanserin is administered to pretreat at least 300 minutes prior to the 2C-B HCl Form C. In some embodiments, the serotonin receptor modulator is pimavanserin and the psychedelic is 2C-B HCl Form C, wherein the pimavanserin is administered to pretreat at least 330 minutes prior to the 2C-B HCl Form C. In some embodiments, the serotonin receptor modulator is pimavanserin and the psychedelic is 2C-B HCl Form C, wherein the pimavanserin is administered to pretreat at least 360 minutes prior to the 2C-B HCl Form C. In some preferred embodiments, the serotonin receptor modulator is pimavanserin and the psychedelic is 2C-B HCl Form C, wherein pimavanserin is administered to pretreat between about 60 minutes and about 180 minutes prior to the administration of the 2C-B HCl Form C.

In some embodiments, the serotonin receptor modulator is nelotanserin and the psychedelic is 2C-B HCl Form C, wherein the nelotanserin is administered to pretreat at least 15 minutes prior to the administration of the 2C-B HCl Form C. In some embodiments, the serotonin receptor modulator is nelotanserin and the psychedelic is 2C-B HCl Form C, wherein the nelotanserin is administered to pretreat at least 30 minutes prior to the 2C-B HCl Form C. In some embodiments, the serotonin receptor modulator is nelotanserin and the psychedelic is 2C-B HCl Form C, wherein the nelotanserin is administered to pretreat between at least 60 minutes and 240 minutes prior to the administration or release of the 2C-B HCl Form C. In some embodiments, the serotonin receptor modulator is nelotanserin and the psychedelic is 2C-B HCl Form C, wherein the nelotanserin is administered to pretreat at least 90 minutes prior to the 2C-B HCl Form C. In some embodiments, the serotonin receptor modulator is nelotanserin and the psychedelic is 2C-B HCl Form C, wherein the nelotanserin is administered to pretreat at least 120 minutes prior to the 2C-B HCl Form C.

In some embodiments, the serotonin receptor modulator is nelotanserin and the psychedelic is 2C-B HCl Form C, wherein the nelotanserin is administered to pretreat at least 150 minutes prior to the 2C-B HCl Form C. In some embodiments, the serotonin receptor modulator is nelotanserin and the psychedelic is 2C-B HCl Form C, wherein the nelotanserin is administered to pretreat between about 15 minutes and about 150 minutes prior to the 2C-B HCl Form C. In some embodiments, the serotonin receptor modulator is nelotanserin and the psychedelic is 2C-B HCl Form C, wherein the nelotanserin is administered to pretreat at least 180 minutes prior to the 2C-B HCl Form C. In some embodiments, the serotonin receptor modulator is nelotanserin and the psychedelic is 2C-B HCl Form C, wherein the nelotanserin is administered to pretreat at least 210 minutes prior to the 2C-B HCl Form C. In some embodiments, the serotonin receptor modulator is nelotanserin and the psychedelic is 2C-B HCl Form C, wherein the nelotanserin is administered to pretreat at least 240 minutes prior to the 2C-B HCl Form C. In some embodiments, the serotonin receptor modulator is nelotanserin and the psychedelic is 2C-B HCl Form C, wherein the nelotanserin is administered to pretreat at least 270 minutes prior to the 2C-B HCl Form C.

In some embodiments, the serotonin receptor modulator is nelotanserin and the psychedelic is 2C-B HCl Form C, wherein the nelotanserin is administered to pretreat at least 300 minutes prior to the 2C-B HCl Form C. In some embodiments, the serotonin receptor modulator is nelotanserin and the psychedelic is 2C-B HCl Form C, wherein the nelotanserin is administered to pretreat at least 330 minutes prior to the 2C-B HCl Form C. In some embodiments, the serotonin receptor modulator is nelotanserin and the psychedelic is 2C-B HCl Form C, wherein the nelotanserin is administered to pretreat at least 360 minutes prior to the 2C-B HCl Form C. In some preferred embodiments, the serotonin receptor modulator is nelotanserin and the psychedelic is 2C-B HCl Form C, wherein nelotanserin is administered to pretreat between about 60 minutes and about 180 minutes prior to the administration of the 2C-B HCl Form C.

In some embodiments, the serotonin receptor modulator is pruvanserin and the psychedelic is 2C-B HCl Form C, wherein the pruvanserin is administered to pretreat at least 15 minutes prior to the administration of the 2C-B HCl Form C. In some embodiments, the serotonin receptor modulator is pruvanserin and the psychedelic is 2C-B HCl Form C, wherein the pruvanserin is administered to pretreat at least 30 minutes prior to the 2C-B HCl Form C. In some embodiments, the serotonin receptor modulator is pruvanserin and the psychedelic is 2C-B HCl Form C, wherein the pruvanserin is administered to pretreat between at least 60 minutes and 240 minutes prior to the administration or release of the 2C-B HCl Form C. In some embodiments, the serotonin receptor modulator is pruvanserin and the psychedelic is 2C-B HCl Form C, wherein the pruvanserin is administered to pretreat at least 90 minutes prior to the 2C-B HCl Form C. In some embodiments, the serotonin receptor modulator is pruvanserin and the psychedelic is 2C-B HCl Form C, wherein the pruvanserin is administered to pretreat at least 120 minutes prior to the 2C-B HCl Form C. In some embodiments, the serotonin receptor modulator is pruvanserin and the psychedelic is 2C-B HCl Form C, wherein the pruvanserin is administered to pretreat at least 150 minutes prior to the 2C-B HCl Form C. In some embodiments, the serotonin receptor modulator is pruvanserin and the psychedelic is 2C-B HCl Form C, wherein the pruvanserin is administered to pretreat between about 15 minutes and about 150 minutes prior to the 2C-B HCl Form C. In some embodiments, the serotonin receptor modulator is pruvanserin and the psychedelic is 2C-B HCl Form C, wherein the pruvanserin is administered to pretreat at least 180 minutes prior to the 2C-B HCl Form C. In some embodiments, the serotonin receptor modulator is pruvanserin and the psychedelic is 2C-B HCl Form C, wherein the pruvanserin is administered to pretreat at least 210 minutes prior to the 2C-B HCl Form C.

In some embodiments, the serotonin receptor modulator is pruvanserin and the psychedelic is 2C-B HCl Form C, wherein the pruvanserin is administered to pretreat at least 240 minutes prior to the 2C-B HCl Form C. In some embodiments, the serotonin receptor modulator is pruvanserin and the psychedelic is 2C-B HCl Form C, wherein the pruvanserin is administered to pretreat at least 270 minutes prior to the 2C-B HCl Form C. In some embodiments, the serotonin receptor modulator is pruvanserin and the psychedelic is 2C-B HCl Form C, wherein the pruvanserin is administered to pretreat at least 300 minutes prior to the 2C-B HCl Form C. In some embodiments, the serotonin receptor modulator is pruvanserin and the psychedelic is 2C-B HCl Form C, wherein the pruvanserin is administered to pretreat at least 330 minutes prior to the 2C-B HCl Form C. In some embodiments, the serotonin receptor modulator is pruvanserin and the psychedelic is 2C-B HCl Form C, wherein the pruvanserin is administered to pretreat at least 360 minutes prior to the 2C-B HCl Form C. In some preferred embodiments, the serotonin receptor modulator is pruvanserin and the psychedelic is 2C-B HCl Form C, wherein pruvanserin is administered to pretreat between about 60 minutes and about 180 minutes prior to the administration of the 2C-B HCl Form C.

In some embodiments, the serotonin receptor modulator is flibanserin and the psychedelic is 2C-B HCl Form C, wherein the flibanserin is administered to pretreat at least 15 minutes prior to the administration of the 2C-B HCl Form C. In some embodiments, the serotonin receptor modulator is flibanserin and the psychedelic is 2C-B HCl Form C, wherein the flibanserin is administered to pretreat at least 30 minutes prior to the 2C-B HCl Form C. In some embodiments, the serotonin receptor modulator is flibanserin and the psychedelic is 2C-B HCl Form C, wherein the flibanserin is administered to pretreat between at least 60 minutes and 240 minutes prior to the administration or release of the 2C-B HCl Form C. In some embodiments, the serotonin receptor modulator is flibanserin and the psychedelic is 2C-B HCl Form C, wherein the flibanserin is administered to pretreat at least 90 minutes prior to the 2C-B HCl Form C. In some embodiments, the serotonin receptor modulator is flibanserin and the psychedelic is 2C-B HCl Form C, wherein the flibanserin is administered to pretreat at least 120 minutes prior to the 2C-B HCl Form C. In some embodiments, the serotonin receptor modulator is flibanserin and the psychedelic is 2C-B HCl Form C, wherein the flibanserin is administered to pretreat at least 150 minutes prior to the 2C-B HCl Form C. In some embodiments, the serotonin receptor modulator is flibanserin and the psychedelic is 2C-B HCl Form C, wherein the flibanserin is administered to pretreat between about 15 minutes and about 150 minutes prior to the 2C-B HCl Form C. In some embodiments, the serotonin receptor modulator is flibanserin and the psychedelic is 2C-B HCl Form C, wherein the flibanserin is administered to pretreat at least 180 minutes prior to the 2C-B HCl Form C. In some embodiments, the serotonin receptor modulator is flibanserin and the psychedelic is 2C-B HCl Form C, wherein the flibanserin is administered to pretreat at least 210 minutes prior to the 2C-B HCl Form C.

In some embodiments, the serotonin receptor modulator is flibanserin and the psychedelic is 2C-B HCl Form C, wherein the flibanserin is administered to pretreat at least 240 minutes prior to the 2C-B HCl Form C. In some embodiments, the serotonin receptor modulator is flibanserin and the psychedelic is 2C-B HCl Form C, wherein the flibanserin is administered to pretreat at least 270 minutes prior to the 2C-B HCl Form C. In some embodiments, the serotonin receptor modulator is flibanserin and the psychedelic is 2C-B HCl Form C, wherein the flibanserin is administered to pretreat at least 300 minutes prior to the 2C-B HCl Form C. In some embodiments, the serotonin receptor modulator is flibanserin and the psychedelic is 2C-B HCl Form C, wherein the flibanserin is administered to pretreat at least 330 minutes prior to the 2C-B HCl Form C. In some embodiments, the serotonin receptor modulator is flibanserin and the psychedelic is 2C-B HCl Form C, wherein the flibanserin is administered to pretreat at least 360 minutes prior to the 2C-B HCl Form C. In some preferred embodiments, the serotonin receptor modulator is flibanserin and the psychedelic is 2C-B HCl Form C, wherein flibanserin is administered to pretreat between about 60 minutes and about 180 minutes prior to the administration of the 2C-B HCl Form C.

In some embodiments, the serotonin receptor modulator is olanzapine and the psychedelic is 2C-B HCl Form C, wherein the olanzapine is administered to pretreat at least 15 minutes prior to the administration of the 2C-B HCl Form C. In some embodiments, the serotonin receptor modulator is olanzapine and the psychedelic is 2C-B HCl Form C, wherein the olanzapine is administered to pretreat at least 30 minutes prior to the 2C-B HCl Form C. In some embodiments, the serotonin receptor modulator is olanzapine and the psychedelic is 2C-B HCl Form C, wherein the olanzapine is administered to pretreat between at least 60 minutes and 240 minutes prior to the administration or release of the 2C-B HCl Form C. In some embodiments, the serotonin receptor modulator is olanzapine and the psychedelic is 2C-B HCl Form C, wherein the olanzapine is administered to pretreat at least 90 minutes prior to the 2C-B HCl Form C. In some embodiments, the serotonin receptor modulator is olanzapine and the psychedelic is 2C-B HCl Form C, wherein the olanzapine is administered to pretreat at least 120 minutes prior to the 2C-B HCl Form C. In some embodiments, the serotonin receptor modulator is olanzapine and the psychedelic is 2C-B HCl Form C, wherein the olanzapine is administered to pretreat at least 150 minutes prior to the 2C-B HCl Form C. In some embodiments, the serotonin receptor modulator is olanzapine and the psychedelic is 2C-B HCl Form C, wherein the olanzapine is administered to pretreat between about 15 minutes and about 150 minutes prior to the 2C-B HCl Form C. In some embodiments, the serotonin receptor modulator is olanzapine and the psychedelic is 2C-B HCl Form C, wherein the olanzapine is administered to pretreat at least 180 minutes prior to the 2C-B HCl Form C. In some embodiments, the serotonin receptor modulator is olanzapine and the psychedelic is 2C-B HCl Form C, wherein the olanzapine is administered to pretreat at least 210 minutes prior to the 2C-B HCl Form C.

In some embodiments, the serotonin receptor modulator is olanzapine and the psychedelic is 2C-B HCl Form C, wherein the olanzapine is administered to pretreat at least 240 minutes prior to the 2C-B HCl Form C. In some embodiments, the serotonin receptor modulator is olanzapine and the psychedelic is 2C-B HCl Form C, wherein the olanzapine is administered to pretreat at least 270 minutes prior to the 2C-B HCl Form C. In some embodiments, the serotonin receptor modulator is olanzapine and the psychedelic is 2C-B HCl Form C, wherein the olanzapine is administered to pretreat at least 300 minutes prior to the 2C-B HCl Form C. In some embodiments, the serotonin receptor modulator is olanzapine and the psychedelic is 2C-B HCl Form C, wherein the olanzapine is administered to pretreat at least 330 minutes prior to the 2C-B HCl Form C. In some embodiments, the serotonin receptor modulator is olanzapine and the psychedelic is 2C-B HCl Form C, wherein the olanzapine is administered to pretreat at least 360 minutes prior to the 2C-B HCl Form C. In some preferred embodiments, the serotonin receptor modulator is olanzapine and the psychedelic is 2C-B HCl Form C, wherein olanzapine is administered to pretreat between about 60 minutes and about 180 minutes prior to the administration of the 2C-B HCl Form C.

In some embodiments, the serotonin receptor modulator is risperidone and the psychedelic is 2C-B HCl Form C, wherein the risperidone is administered to pretreat at least 15 minutes prior to the administration of the 2C-B HCl Form C. In some embodiments, the serotonin receptor modulator is risperidone and the psychedelic is 2C-B HCl Form C, wherein the risperidone is administered to pretreat at least 30 minutes prior to the 2C-B HCl Form C. In some embodiments, the serotonin receptor modulator is risperidone and the psychedelic is 2C-B HCl Form C, wherein the risperidone is administered to pretreat between at least 60 minutes and 240 minutes prior to the administration or release of the 2C-B HCl Form C. In some embodiments, the serotonin receptor modulator is risperidone and the psychedelic is 2C-B HCl Form C, wherein the risperidone is administered to pretreat at least 90 minutes prior to the 2C-B HCl Form C. In some embodiments, the serotonin receptor modulator is risperidone and the psychedelic is 2C-B HCl Form C, wherein the risperidone is administered to pretreat at least 120 minutes prior to the 2C-B HCl Form C. In some embodiments, the serotonin receptor modulator is risperidone and the psychedelic is 2C-B HCl Form C, wherein the risperidone is administered to pretreat at least 150 minutes prior to the 2C-B HCl Form C. In some embodiments, the serotonin receptor modulator is risperidone and the psychedelic is 2C-B HCl Form C, wherein the risperidone is administered to pretreat between about 15 minutes and about 150 minutes prior to the 2C-B HCl Form C. In some embodiments, the serotonin receptor modulator is risperidone and the psychedelic is 2C-B HCl Form C, wherein the risperidone is administered to pretreat at least 180 minutes prior to the 2C-B HCl Form C. In some embodiments, the serotonin receptor modulator is risperidone and the psychedelic is 2C-B HCl Form C, wherein the risperidone is administered to pretreat at least 210 minutes prior to the 2C-B HCl Form C.

In some embodiments, the serotonin receptor modulator is risperidone and the psychedelic is 2C-B HCl Form C, wherein the risperidone is administered to pretreat at least 240 minutes prior to the 2C-B HCl Form C. In some embodiments, the serotonin receptor modulator is risperidone and the psychedelic is 2C-B HCl Form C, wherein the risperidone is administered to pretreat at least 270 minutes prior to the 2C-B HCl Form C. In some embodiments, the serotonin receptor modulator is risperidone and the psychedelic is 2C-B HCl Form C, wherein the risperidone is administered to pretreat at least 300 minutes prior to the 2C-B HCl Form C. In some embodiments, the serotonin receptor modulator is risperidone and the psychedelic is 2C-B HCl Form C, wherein the risperidone is administered to pretreat at least 330 minutes prior to the 2C-B HCl Form C. In some embodiments, the serotonin receptor modulator is risperidone and the psychedelic is 2C-B HCl Form C, wherein the risperidone is administered to pretreat at least 360 minutes prior to the 2C-B HCl Form C. In some preferred embodiments, the serotonin receptor modulator is risperidone and the psychedelic is 2C-B HCl Form C, wherein risperidone is administered to pretreat between about 60 minutes and about 180 minutes prior to the administration of the 2C-B HCl Form C.

In some embodiments, the serotonin receptor modulator is quetiapine and the psychedelic is 2C-B HCl Form C, wherein the quetiapine is administered to pretreat at least 15 minutes prior to the administration of the 2C-B HCl Form C. In some embodiments, the serotonin receptor modulator is quetiapine and the psychedelic is 2C-B HCl Form C, wherein the quetiapine is administered to pretreat at least 30 minutes prior to the 2C-B HCl Form C. In some embodiments, the serotonin receptor modulator is quetiapine and the psychedelic is 2C-B HCl Form C, wherein the quetiapine is administered to pretreat between at least 60 minutes and 240 minutes prior to the administration or release of the 2C-B HCl Form C. In some embodiments, the serotonin receptor modulator is quetiapine and the psychedelic is 2C-B HCl Form C, wherein the quetiapine is administered to pretreat at least 90 minutes prior to the 2C-B HCl Form C. In some embodiments, the serotonin receptor modulator is quetiapine and the psychedelic is 2C-B HCl Form C, wherein the quetiapine is administered to pretreat at least 120 minutes prior to the 2C-B HCl Form C. In some embodiments, the serotonin receptor modulator is quetiapine and the psychedelic is 2C-B HCl Form C, wherein the quetiapine is administered to pretreat at least 150 minutes prior to the 2C-B HCl Form C. In some embodiments, the serotonin receptor modulator is quetiapine and the psychedelic is 2C-B HCl Form C, wherein the quetiapine is administered to pretreat between about 15 minutes and about 150 minutes prior to the 2C-B HCl Form C. In some embodiments, the serotonin receptor modulator is quetiapine and the psychedelic is 2C-B HCl Form C, wherein the quetiapine is administered to pretreat at least 180 minutes prior to the 2C-B HCl Form C. In some embodiments, the serotonin receptor modulator is quetiapine and the psychedelic is 2C-B HCl Form C, wherein the quetiapine is administered to pretreat at least 210 minutes prior to the 2C-B HCl Form C.

In some embodiments, the serotonin receptor modulator is quetiapine and the psychedelic is 2C-B HCl Form C, wherein the quetiapine is administered to pretreat at least 240 minutes prior to the 2C-B HCl Form C. In some embodiments, the serotonin receptor modulator is quetiapine and the psychedelic is 2C-B HCl Form C, wherein the quetiapine is administered to pretreat at least 270 minutes prior to the 2C-B HCl Form C. In some embodiments, the serotonin receptor modulator is quetiapine and the psychedelic is 2C-B HCl Form C, wherein the quetiapine is administered to pretreat at least 300 minutes prior to the 2C-B HCl Form C. In some embodiments, the serotonin receptor modulator is quetiapine and the psychedelic is 2C-B HCl Form C, wherein the quetiapine is administered to pretreat at least 330 minutes prior to the 2C-B HCl Form C. In some embodiments, the serotonin receptor modulator is quetiapine and the psychedelic is 2C-B HCl Form C, wherein the quetiapine is administered to pretreat at least 360 minutes prior to the 2C-B HCl Form C. In some preferred embodiments, the serotonin receptor modulator is quetiapine and the psychedelic is 2C-B HCl Form C, wherein quetiapine is administered to pretreat between about 60 minutes and about 180 minutes prior to the administration of the 2C-B HCl Form C.

In certain embodiments, such as those described above a 2C-B or 2C-B HCl form disclosed herein is co-administered with a serotonin receptor modulator in the same or in separate compositions. In one embodiment, the serotonin receptor modulator is administered after the 2C-B or 2C-B HCl form disclosed herein. In one embodiment, the 2C-B or 2C-B HCl form disclosed herein is administered in a modified release formulation such that the subject is effectively post-treated with serotonin receptor modulator post to release of an effective amount of the 2C-B. In some embodiments, the serotonin receptor modulator is part of a single fixed dose formulation that releases the 2C-B first followed by serotonin receptor modulator on two different release profiles. In another embodiment, the 2C-B or 2C-B HCl form disclosed herein is administered first as a single dosage and, after a length of time, serotonin receptor modulator is administered as a second dosage separate from the first dosage. Thus, in some embodiments, the serotonin receptor modulator is administered or released from a composition provided herein after the administration and/or release of the psychedelic. This allows post-treatment to attenuate activation of the serotonin receptor by the psychedelic.

In some embodiments, the serotonin receptor modulator is administered or released from the composition provided herein to post-treat a subject by at least about at about 5 minutes, 10 minutes, 20 minutes, 30 minutes, 40 minutes, 50 minutes, 1 hour, 1.25 hours, 1.5 hours, 2 hours, or 3 hours after the release of the psychedelic. In some embodiments, the serotonin receptor modulator attenuates the activation of the serotonin receptor when the serotonin receptor modulator is used to post-treat at most about 3 hours, 4 hours, 5 hours, 6 hours, 7 hours, 8 hours, 9 hours, or more than 9 hours after the release of the psychedelic. In some embodiments, the serotonin receptor modulator attenuates the activation of the serotonin receptor when the serotonin receptor modulator is used to post-treat in a range of about 5 minutes to about 3 hours, about 10 minutes to about 3 hours, about 20 minutes to about 3 hours, about 30 minutes to about 3 hours, about 40 minutes to about 3 hours, about 50 minutes to about 3 hours, about 1 hour to about 3 hours, about 5 minutes to about 2 hours, about 10 minutes to about 2 hours, about 20 minutes to about 2 hours, about 30 minutes to about 2 hours, about 40 minutes to about 2 hours, about 50 minutes to about 2 hours, about 1 hour to about 2 hours, about 5 minutes to about 1 hour, about 10 minutes to about 1 hour, about 20 minutes to about 1 hour, about 30 minutes to about 1 hour, about 40 minutes to about 1 hour, or about 50 minutes to about 1 hour after the release of the psychedelic.

In a preferred embodiment, the serotonin receptor modulator is administered at about 1 hour to about 3 hours after the administration of the psychedelic.

In some embodiments, the serotonin receptor modulator is eplivanserin and the psychedelic is 2C-B HCl, wherein the eplivanserin is administered to post-treat at least 15 minutes after the administration of the 2C-B HCl form disclosed herein, including those described in Table 3. In some embodiments, the serotonin receptor modulator is eplivanserin and the psychedelic is 2C-B HCl, wherein the eplivanserin is administered to post-treat between at least 30 minutes after and 360 minutes after the release or administration of the 2C-B HCl form disclosed herein, including those described in Table 3. In some embodiments, the serotonin receptor modulator is eplivanserin and the psychedelic is 2C-B HCl, wherein the eplivanserin is administered to post-treat between at least 60 minutes after and 360 minutes after the release or administration of the 2C-B HCl form disclosed herein, including those described in Table 3. In some embodiments, the serotonin receptor modulator is eplivanserin and the psychedelic is 2C-B HCl, wherein the eplivanserin is administered to post-treat between at least 90 minutes and 240 minutes after the 2C-B HCl form disclosed herein, including those described in Table 3. In some embodiments, the serotonin receptor modulator is eplivanserin and the psychedelic is 2C-B HCl, wherein the eplivanserin is administered to post-treat at least 120 minutes after the 2C-B HCl form disclosed herein, including those described in Table 3.

In some embodiments, the serotonin receptor modulator is eplivanserin and the psychedelic is 2C-B HCl, wherein the eplivanserin is administered to post-treat at least 150 minutes after the 2C-B HCl form disclosed herein, including those described in Table 3. In some embodiments, the serotonin receptor modulator is eplivanserin and the psychedelic is 2C-B HCl, wherein the eplivanserin is administered to post-treat between about 15 minutes and about 150 minutes after the 2C-B HCl form disclosed herein, including those described in Table 3. In some embodiments, the serotonin receptor modulator is eplivanserin and the psychedelic is 2C-B HCl, wherein the eplivanserin is administered to post-treat at least 180 minutes after the 2C-B HCl form disclosed herein, including those described in Table 3. In some embodiments, the serotonin receptor modulator is eplivanserin and the psychedelic is 2C-B HCl, wherein the eplivanserin is administered to post-treat at least 210 minutes after the 2C-B HCl form disclosed herein, including those described in Table 3. In some embodiments, the serotonin receptor modulator is eplivanserin and the psychedelic is 2C-B HCl, wherein the eplivanserin is administered to post-treat at least 240 minutes after the 2C-B HCl form disclosed herein, including those described in Table 3.

In some embodiments, the serotonin receptor modulator is eplivanserin and the psychedelic is 2C-B HCl, wherein the eplivanserin is administered to post-treat at least 270 minutes after the 2C-B HCl form disclosed herein, including those described in Table 3. In some embodiments, the serotonin receptor modulator is eplivanserin and the psychedelic is 2C-B HCl, wherein the eplivanserin is administered to post-treat at least 300 minutes after the 2C-B HCl form disclosed herein, including those described in Table 3. In some embodiments, the serotonin receptor modulator is eplivanserin and the psychedelic is 2C-B HCl, wherein the eplivanserin is administered to post-treat at least 330 minutes after the 2C-B HCl form disclosed herein, including those described in Table 3. In some embodiments, the serotonin receptor modulator is eplivanserin and the psychedelic is 2C-B HCl, wherein the eplivanserin is administered to post-treat at least 360 minutes after the 2C-B HCl form disclosed herein, including those described in Table 3.

In some preferred embodiments, the serotonin receptor modulator is eplivanserin and the psychedelic is 2C-B HCl, wherein eplivanserin is administered to post-treat between about 60 minutes and about 180 minutes after the administration of the 2C-B HCl form disclosed herein, including those described in Table 3.

In some embodiments, the serotonin receptor modulator is volinanserin and the psychedelic is 2C-B HCl, wherein the volinanserin is administered to post-treat a subject between at least 15 minutes and 360 minutes after the administration or release of the 2C-B HCl form disclosed herein, including those described in Table 3. In some embodiments, the serotonin receptor modulator is volinanserin and the psychedelic is 2C-B HCl, wherein the volinanserin is administered to post-treat between at least 30 minutes and 360 minutes after the administration or release of the 2C-B HCl form disclosed herein, including those described in Table 3. In some embodiments, the serotonin receptor modulator is volinanserin and the psychedelic is 2C-B HCl, wherein the volinanserin is administered to post-treat between at least 60 minutes and 240 minutes after the administration or release of the 2C-B HCl form disclosed herein, including those described in Table 3. In some embodiments, the serotonin receptor modulator is volinanserin and the psychedelic is 2C-B HCl, wherein the volinanserin is administered to post-treat at least 90 minutes after 2C-B HCl. In some embodiments, the serotonin receptor modulator is volinanserin and the psychedelic is 2C-B HCl, wherein the volinanserin is administered to post-treat at least 120 minutes after the 2C-B HCl form disclosed herein, including those described in Table 3.

In some embodiments, the serotonin receptor modulator is volinanserin and the psychedelic is 2C-B HCl, wherein the volinanserin is administered to post-treat at least 150 minutes after the 2C-B HCl form disclosed herein, including those described in Table 3. In some embodiments, the serotonin receptor modulator is volinanserin and the psychedelic is 2C-B HCl, wherein the volinanserin is administered to post-treat between about 15 minutes and about 150 minutes after the 2C-B HCl form disclosed herein, including those described in Table 3. In some embodiments, the serotonin receptor modulator is volinanserin and the psychedelic is 2C-B HCl, wherein the volinanserin is administered to post-treat at least 180 minutes after the 2C-B HCl form disclosed herein, including those described in Table 3. In some embodiments, the serotonin receptor modulator is volinanserin and the psychedelic is 2C-B HCl, wherein the volinanserin is administered to post-treat at least 210 minutes after the 2C-B HCl form disclosed herein, including those described in Table 3. In some embodiments, the serotonin receptor modulator is volinanserin and the psychedelic is 2C-B HCl, wherein the volinanserin is administered to post-treat at least 240 minutes after the 2C-B HCl form disclosed herein, including those described in Table 3. In some embodiments, the serotonin receptor modulator is volinanserin and the psychedelic is 2C-B HCl, wherein the volinanserin is administered to post-treat at least 270 minutes after the 2C-B HCl form disclosed herein, including those described in Table 3. In some embodiments, the serotonin receptor modulator is volinanserin and the psychedelic is 2C-B HCl, wherein the volinanserin is administered to post-treat at least 300 minutes after the 2C-B HCl form disclosed herein, including those described in Table 3. In some embodiments, the serotonin receptor modulator is volinanserin and the psychedelic is 2C-B HCl, wherein the volinanserin is administered to post-treat at least 330 minutes after the 2C-B HCl form disclosed herein, including those described in Table 3. In some embodiments, the serotonin receptor modulator is volinanserin and the psychedelic is 2C-B HCl, wherein the volinanserin is administered to post-treat at least 360 minutes after the 2C-B HCl form disclosed herein, including those described in Table 3. In some preferred embodiments, the serotonin receptor modulator is volinanserin and the psychedelic is 2C-B HCl, wherein volinanserin is administered to post-treat between about 60 minutes and about 180 minutes after the administration of the 2C-B HCl form disclosed herein, including those described in Table 3.

In some embodiments, the serotonin receptor modulator is ketanserin and the psychedelic is 2C-B HCl, wherein the ketanserin is administered to post-treat at least 15 minutes after the administration of the 2C-B HCl form disclosed herein, including those described in Table 3. In some embodiments, the serotonin receptor modulator is ketanserin and the psychedelic is 2C-B HCl, wherein the ketanserin is administered to post-treat between at least 30 minutes and 360 minutes after the administration or release of the 2C-B HCl form disclosed herein, including those described in Table 3. In some embodiments, the serotonin receptor modulator is ketanserin and the psychedelic is 2C-B HCl, wherein the ketanserin is administered to post-treat between at least 60 minutes and 240 minutes after the administration or release of the 2C-B HCl form disclosed herein, including those described in Table 3. In some embodiments, the serotonin receptor modulator is ketanserin and the psychedelic is 2C-B HCl, wherein the ketanserin is administered to post-treat at least 90 minutes after the 2C-B HCl form disclosed herein, including those described in Table 3. In some embodiments, the serotonin receptor modulator is ketanserin and the psychedelic is 2C-B HCl, wherein the ketanserin is administered to post-treat at least 120 minutes after the 2C-B HCl form disclosed herein, including those described in Table 3. In some embodiments, the serotonin receptor modulator is ketanserin and the psychedelic is 2C-B HCl, wherein the ketanserin is administered to post-treat at least 150 minutes after the 2C-B HCl form disclosed herein, including those described in Table 3. In some embodiments, the serotonin receptor modulator is ketanserin and the psychedelic is 2C-B HCl, wherein the ketanserin is administered to post-treat between about 15 minutes and about 150 minutes after the 2C-B HCl form disclosed herein, including those described in Table 3. In some embodiments, the serotonin receptor modulator is ketanserin and the psychedelic is 2C-B HCl, wherein the ketanserin is administered to post-treat at least 180 minutes after the 2C-B HCl form disclosed herein, including those described in Table 3. In some embodiments, the serotonin receptor modulator is ketanserin and the psychedelic is 2C-B HCl, wherein the ketanserin is administered to post-treat at least 210 minutes after the 2C-B HCl form disclosed herein, including those described in Table 3. In some embodiments, the serotonin receptor modulator is ketanserin and the psychedelic is 2C-B HCl, wherein the ketanserin is administered to post-treat at least 240 minutes after the 2C-B HCl form disclosed herein, including those described in Table 3. In some embodiments, the serotonin receptor modulator is ketanserin and the psychedelic is 2C-B HCl, wherein the ketanserin is administered to post-treat at least 270 minutes after the 2C-B HCl form disclosed herein, including those described in Table 3. In some embodiments, the serotonin receptor modulator is ketanserin and the psychedelic is 2C-B HCl, wherein the ketanserin is administered to post-treat at least 300 minutes after the 2C-B HCl form disclosed herein, including those described in Table 3. In some embodiments, the serotonin receptor modulator is ketanserin and the psychedelic is 2C-B HCl, wherein the ketanserin is administered to post-treat at least 330 minutes after the 2C-B HCl form disclosed herein, including those described in Table 3. In some embodiments, the serotonin receptor modulator is ketanserin and the psychedelic is 2C-B HCl, wherein the ketanserin is administered to post-treat at least 360 minutes after the 2C-B HCl form disclosed herein, including those described in Table 3. In some preferred embodiments, the serotonin receptor modulator is ketanserin and the psychedelic is 2C-B HCl, wherein ketanserin is administered to post-treat between about 60 minutes and about 180 minutes after the administration of the 2C-B HCl form disclosed herein, including those described in Table 3.

In some embodiments, the serotonin receptor modulator is ritanserin and the psychedelic is 2C-B HCl, wherein the ritanserin is administered to post-treat at least 15 minutes after the administration of the 2C-B HCl form disclosed herein, including those described in Table 3. In some embodiments, the serotonin receptor modulator is ritanserin and the psychedelic is 2C-B HCl, wherein the ritanserin is administered to post-treat at least 30 minutes after the 2C-B HCl form disclosed herein, including those described in Table 3. In some embodiments, the serotonin receptor modulator is ritanserin and the psychedelic is 2C-B HCl, wherein the ritanserin is administered to post-treat between at least 60 minutes and 240 minutes after the administration or release of the 2C-B HCl form disclosed herein, including those described in Table 3. In some embodiments, the serotonin receptor modulator is ritanserin and the psychedelic is 2C-B HCl, wherein the ritanserin is administered to post-treat at least 90 minutes after the 2C-B HCl form disclosed herein, including those described in Table 3. In some embodiments, the serotonin receptor modulator is ritanserin and the psychedelic is 2C-B HCl, wherein the ritanserin is administered to post-treat at least 120 minutes after the 2C-B HCl form disclosed herein, including those described in Table 3. In some embodiments, the serotonin receptor modulator is ritanserin and the psychedelic is 2C-B HCl, wherein the ritanserin is administered to post-treat at least 150 minutes after the 2C-B HCl form disclosed herein, including those described in Table 3. In some embodiments, the serotonin receptor modulator is ritanserin and the psychedelic is 2C-B HCl, wherein the ritanserin is administered to post-treat between about 15 minutes and about 150 minutes after the 2C-B HCl form disclosed herein, including those described in Table 3. In some embodiments, the serotonin receptor modulator is ritanserin and the psychedelic is 2C-B HCl, wherein the ritanserin is administered to post-treat at least 180 minutes after the 2C-B HCl form disclosed herein, including those described in Table 3. In some embodiments, the serotonin receptor modulator is ritanserin and the psychedelic is 2C-B HCl, wherein the ritanserin is administered to post-treat at least 210 minutes after the 2C-B HCl form disclosed herein, including those described in Table 3. In some embodiments, the serotonin receptor modulator is ritanserin and the psychedelic is 2C-B HCl, wherein the ritanserin is administered to post-treat at least 240 minutes after the 2C-B HCl form disclosed herein, including those described in Table 3. In some embodiments, the serotonin receptor modulator is ritanserin and the psychedelic is 2C-B HCl, wherein the ritanserin is administered to post-treat at least 270 minutes after the 2C-B HCl form disclosed herein, including those described in Table 3. In some embodiments, the serotonin receptor modulator is ritanserin and the psychedelic is 2C-B HCl, wherein the ritanserin is administered to post-treat at least 300 minutes after the 2C-B HCl form disclosed herein, including those described in Table 3. In some embodiments, the serotonin receptor modulator is ritanserin and the psychedelic is 2C-B HCl, wherein the ritanserin is administered to post-treat at least 330 minutes after the 2C-B HCl form disclosed herein, including those described in Table 3. In some embodiments, the serotonin receptor modulator is ritanserin and the psychedelic is 2C-B HCl, wherein the ritanserin is administered to post-treat at least 360 minutes after the 2C-B HCl form disclosed herein, including those described in Table 3. In some preferred embodiments, the serotonin receptor modulator is ritanserin and the psychedelic is 2C-B HCl, wherein ritanserin is administered to post-treat between about 60 minutes and about 180 minutes after the administration of the 2C-B HCl form disclosed herein, including those described in Table 3.

In some embodiments, the serotonin receptor modulator is pimavanserin and the psychedelic is 2C-B HCl, wherein the pimavanserin is administered to post-treat at least 15 minutes after the administration of the 2C-B HCl form disclosed herein, including those described in Table 3. In some embodiments, the serotonin receptor modulator is pimavanserin and the psychedelic is 2C-B HCl, wherein the pimavanserin is administered to post-treat at least 30 minutes after the 2C-B HCl form disclosed herein, including those described in Table 3. In some embodiments, the serotonin receptor modulator is pimavanserin and the psychedelic is 2C-B HCl, wherein the pimavanserin is administered to post-treat between at least 60 minutes and 240 minutes after the administration or release of the 2C-B HCl form disclosed herein, including those described in Table 3. In some embodiments, the serotonin receptor modulator is pimavanserin and the psychedelic is 2C-B HCl, wherein the pimavanserin is administered to post-treat at least 90 minutes after the 2C-B HCl form disclosed herein, including those described in Table 3. In some embodiments, the serotonin receptor modulator is pimavanserin and the psychedelic is 2C-B HCl, wherein the pimavanserin is administered to post-treat at least 120 minutes after the 2C-B HCl form disclosed herein, including those described in Table 3. In some embodiments, the serotonin receptor modulator is pimavanserin and the psychedelic is 2C-B HCl, wherein the pimavanserin is administered to post-treat at least 150 minutes after the 2C-B HCl form disclosed herein, including those described in Table 3. In some embodiments, the serotonin receptor modulator is pimavanserin and the psychedelic is 2C-B HCl, wherein the pimavanserin is administered to post-treat between about 15 minutes and about 150 minutes after the 2C-B HCl form disclosed herein, including those described in Table 3. In some embodiments, the serotonin receptor modulator is pimavanserin and the psychedelic is 2C-B HCl, wherein the pimavanserin is administered to post-treat at least 180 minutes after the 2C-B HCl form disclosed herein, including those described in Table 3. In some embodiments, the serotonin receptor modulator is pimavanserin and the psychedelic is 2C-B HCl, wherein the pimavanserin is administered to post-treat at least 210 minutes after the 2C-B HCl form disclosed herein, including those described in Table 3. In some embodiments, the serotonin receptor modulator is pimavanserin and the psychedelic is 2C-B HCl, wherein the pimavanserin is administered to post-treat at least 240 minutes after the 2C-B HCl form disclosed herein, including those described in Table 3. In some embodiments, the serotonin receptor modulator is pimavanserin and the psychedelic is 2C-B HCl, wherein the pimavanserin is administered to post-treat at least 270 minutes after the 2C-B HCl form disclosed herein, including those described in Table 3. In some embodiments, the serotonin receptor modulator is pimavanserin and the psychedelic is 2C-B HCl, wherein the pimavanserin is administered to post-treat at least 300 minutes after the 2C-B HCl form disclosed herein, including those described in Table 3. In some embodiments, the serotonin receptor modulator is pimavanserin and the psychedelic is 2C-B HCl, wherein the pimavanserin is administered to post-treat at least 330 minutes after the 2C-B HCl form disclosed herein, including those described in Table 3. In some embodiments, the serotonin receptor modulator is pimavanserin and the psychedelic is 2C-B HCl, wherein the pimavanserin is administered to post-treat at least 360 minutes after the 2C-B HCl form disclosed herein, including those described in Table 3. In some preferred embodiments, the serotonin receptor modulator is pimavanserin and the psychedelic is 2C-B HCl, wherein pimavanserin is administered to post-treat between about 60 minutes and about 180 minutes after the administration of the 2C-B HCl form disclosed herein, including those described in Table 3.

In some embodiments, the serotonin receptor modulator is nelotanserin and the psychedelic is 2C-B HCl, wherein the nelotanserin is administered to post-treat at least 15 minutes after the administration of the 2C-B HCl form disclosed herein, including those described in Table 3. In some embodiments, the serotonin receptor modulator is nelotanserin and the psychedelic is 2C-B HCl, wherein the nelotanserin is administered to post-treat at least 30 minutes after the 2C-B HCl form disclosed herein, including those described in Table 3. In some embodiments, the serotonin receptor modulator is nelotanserin and the psychedelic is 2C-B HCl, wherein the nelotanserin is administered to post-treat between at least 60 minutes and 240 minutes after the administration or release of the 2C-B HCl form disclosed herein, including those described in Table 3. In some embodiments, the serotonin receptor modulator is nelotanserin and the psychedelic is 2C-B HCl, wherein the nelotanserin is administered to post-treat at least 90 minutes after the 2C-B HCl form disclosed herein, including those described in Table 3. In some embodiments, the serotonin receptor modulator is nelotanserin and the psychedelic is 2C-B HCl, wherein the nelotanserin is administered to post-treat at least 120 minutes after the 2C-B HCl form disclosed herein, including those described in Table 3.

In some embodiments, the serotonin receptor modulator is nelotanserin and the psychedelic is 2C-B HCl, wherein the nelotanserin is administered to post-treat at least 150 minutes after the 2C-B HCl form disclosed herein, including those described in Table 3. In some embodiments, the serotonin receptor modulator is nelotanserin and the psychedelic is 2C-B HCl, wherein the nelotanserin is administered to post-treat between about 15 minutes and about 150 minutes after the 2C-B HCl form disclosed herein, including those described in Table 3. In some embodiments, the serotonin receptor modulator is nelotanserin and the psychedelic is 2C-B HCl, wherein the nelotanserin is administered to post-treat at least 180 minutes after the 2C-B HCl form disclosed herein, including those described in Table 3. In some embodiments, the serotonin receptor modulator is nelotanserin and the psychedelic is 2C-B HCl, wherein the nelotanserin is administered to post-treat at least 210 minutes after the 2C-B HCl form disclosed herein, including those described in Table 3. In some embodiments, the serotonin receptor modulator is nelotanserin and the psychedelic is 2C-B HCl, wherein the nelotanserin is administered to post-treat at least 240 minutes after the 2C-B HCl form disclosed herein, including those described in Table 3. In some embodiments, the serotonin receptor modulator is nelotanserin and the psychedelic is 2C-B HCl, wherein the nelotanserin is administered to post-treat at least 270 minutes after the 2C-B HCl form disclosed herein, including those described in Table 3.

In some embodiments, the serotonin receptor modulator is nelotanserin and the psychedelic is 2C-B HCl, wherein the nelotanserin is administered to post-treat at least 300 minutes after the 2C-B HCl form disclosed herein, including those described in Table 3. In some embodiments, the serotonin receptor modulator is nelotanserin and the psychedelic is 2C-B HCl, wherein the nelotanserin is administered to post-treat at least 330 minutes after the 2C-B HCl form disclosed herein, including those described in Table 3. In some embodiments, the serotonin receptor modulator is nelotanserin and the psychedelic is 2C-B HCl, wherein the nelotanserin is administered to post-treat at least 360 minutes after the 2C-B HCl form disclosed herein, including those described in Table 3. In some preferred embodiments, the serotonin receptor modulator is nelotanserin and the psychedelic is 2C-B HCl, wherein nelotanserin is administered to post-treat between about 60 minutes and about 180 minutes after the administration of the 2C-B HCl form disclosed herein, including those described in Table 3.

In some embodiments, the serotonin receptor modulator is pruvanserin and the psychedelic is 2C-B HCl, wherein the pruvanserin is administered to post-treat at least 15 minutes after the administration of the 2C-B HCl form disclosed herein, including those described in Table 3. In some embodiments, the serotonin receptor modulator is pruvanserin and the psychedelic is 2C-B HCl, wherein the pruvanserin is administered to post-treat at least 30 minutes after the 2C-B HCl form disclosed herein, including those described in Table 3. In some embodiments, the serotonin receptor modulator is pruvanserin and the psychedelic is 2C-B HCl, wherein the pruvanserin is administered to post-treat between at least 60 minutes and 240 minutes after the administration or release of the 2C-B HCl form disclosed herein, including those described in Table 3. In some embodiments, the serotonin receptor modulator is pruvanserin and the psychedelic is 2C-B HCl, wherein the pruvanserin is administered to post-treat at least 90 minutes after the 2C-B HCl form disclosed herein, including those described in Table 3. In some embodiments, the serotonin receptor modulator is pruvanserin and the psychedelic is 2C-B HCl, wherein the pruvanserin is administered to post-treat at least 120 minutes after the 2C-B HCl form disclosed herein, including those described in Table 3. In some embodiments, the serotonin receptor modulator is pruvanserin and the psychedelic is 2C-B HCl, wherein the pruvanserin is administered to post-treat at least 150 minutes after the 2C-B HCl form disclosed herein, including those described in Table 3. In some embodiments, the serotonin receptor modulator is pruvanserin and the psychedelic is 2C-B HCl, wherein the pruvanserin is administered to post-treat between about 15 minutes and about 150 minutes after the 2C-B HCl form disclosed herein, including those described in Table 3. In some embodiments, the serotonin receptor modulator is pruvanserin and the psychedelic is 2C-B HCl, wherein the pruvanserin is administered to post-treat at least 180 minutes after the 2C-B HCl form disclosed herein, including those described in Table 3. In some embodiments, the serotonin receptor modulator is pruvanserin and the psychedelic is 2C-B HCl, wherein the pruvanserin is administered to post-treat at least 210 minutes after the 2C-B HCl form disclosed herein, including those described in Table 3.

In some embodiments, the serotonin receptor modulator is pruvanserin and the psychedelic is 2C-B HCl, wherein the pruvanserin is administered to post-treat at least 240 minutes after the 2C-B HCl form disclosed herein, including those described in Table 3. In some embodiments, the serotonin receptor modulator is pruvanserin and the psychedelic is 2C-B HCl, wherein the pruvanserin is administered to post-treat at least 270 minutes after the 2C-B HCl form disclosed herein, including those described in Table 3. In some embodiments, the serotonin receptor modulator is pruvanserin and the psychedelic is 2C-B HCl, wherein the pruvanserin is administered to post-treat at least 300 minutes after the 2C-B HCl form disclosed herein, including those described in Table 3. In some embodiments, the serotonin receptor modulator is pruvanserin and the psychedelic is 2C-B HCl, wherein the pruvanserin is administered to post-treat at least 330 minutes after the 2C-B HCl form disclosed herein, including those described in Table 3. In some embodiments, the serotonin receptor modulator is pruvanserin and the psychedelic is 2C-B HCl, wherein the pruvanserin is administered to post-treat at least 360 minutes after the 2C-B HCl form disclosed herein, including those described in Table 3. In some preferred embodiments, the serotonin receptor modulator is pruvanserin and the psychedelic is 2C-B HCl, wherein pruvanserin is administered to post-treat between about 60 minutes and about 180 minutes after the administration of the 2C-B HCl form disclosed herein, including those described in Table 3.

In some embodiments, the serotonin receptor modulator is flibanserin, wherein the flibanserin is administered to post-treat at least 15 minutes post to the administration of the 2C-B HCl form disclosed herein, including those described in Table 3. In some embodiments, the serotonin receptor modulator is flibanserin, wherein the flibanserin is administered to post-treat at least 30 minutes post to the 2C-B HCl form disclosed herein, including those described in Table 3. In some embodiments, the serotonin receptor modulator is flibanserin, wherein the flibanserin is administered to post-treat between at least 60 minutes and 240 minutes post to the administration or release of the 2C-B HCl form disclosed herein, including those described in Table 3. In some embodiments, the serotonin receptor modulator is flibanserin, wherein the flibanserin is administered to post-treat at least 90 minutes post to the 2C-B HCl form disclosed herein, including those described in Table 3. In some embodiments, the serotonin receptor modulator is flibanserin, wherein the flibanserin is administered to post-treat at least 120 minutes post to the 2C-B HCl form disclosed herein, including those described in Table 3. In some embodiments, the serotonin receptor modulator is flibanserin, wherein the flibanserin is administered to post-treat at least 150 minutes post to the 2C-B HCl form disclosed herein, including those described in Table 3. In some embodiments, the serotonin receptor modulator is flibanserin, wherein the flibanserin is administered to post-treat between about 15 minutes and about 150 minutes post to the 2C-B HCl form disclosed herein, including those described in Table 3. In some embodiments, the serotonin receptor modulator is flibanserin, wherein the flibanserin is administered to post-treat at least 180 minutes post to the 2C-B HCl form disclosed herein, including those described in Table 3. In some embodiments, the serotonin receptor modulator is flibanserin, wherein the flibanserin is administered to post-treat at least 210 minutes post to the 2C-B HCl form disclosed herein, including those described in Table 3.

In some embodiments, the serotonin receptor modulator is flibanserin, wherein the flibanserin is administered to post-treat at least 240 minutes post to the 2C-B HCl form disclosed herein, including those described in Table 3. In some embodiments, the serotonin receptor modulator is flibanserin, wherein the flibanserin is administered to post-treat at least 270 minutes post to the 2C-B HCl form disclosed herein, including those described in Table 3. In some embodiments, the serotonin receptor modulator is flibanserin, wherein the flibanserin is administered to post-treat at least 300 minutes post to the 2C-B HCl form disclosed herein, including those described in Table 3. In some embodiments, the serotonin receptor modulator is flibanserin, wherein the flibanserin is administered to post-treat at least 330 minutes post to the 2C-B HCl form disclosed herein, including those described in Table 3. In some embodiments, the serotonin receptor modulator is flibanserin, wherein the flibanserin is administered to post-treat at least 360 minutes post to the 2C-B HCl form disclosed herein, including those described in Table 3. In some preferred embodiments, the serotonin receptor modulator is flibanserin, wherein flibanserin is administered to post-treat between about 60 minutes and about 180 minutes post to the administration of the 2C-B HCl form disclosed herein, including those described in Table 3. In some embodiments, the serotonin receptor modulator is olanzapine, wherein the olanzapine is administered to post-treat at least 15 minutes post to the administration of the 2C-B HCl form disclosed herein, including those described in Table 3. In some embodiments, the serotonin receptor modulator is olanzapine, wherein the olanzapine is administered to post-treat at least 30 minutes post to the 2C-B HCl form disclosed herein, including those described in Table 3. In some embodiments, the serotonin receptor modulator is olanzapine, wherein the olanzapine is administered to post-treat between at least 60 minutes and 240 minutes post to the administration or release of the 2C-B HCl form disclosed herein, including those described in Table 3. In some embodiments, the serotonin receptor modulator is olanzapine, wherein the olanzapine is administered to post-treat at least 90 minutes post to the 2C-B HCl form disclosed herein, including those described in Table 3. In some embodiments, the serotonin receptor modulator is olanzapine, wherein the olanzapine is administered to post-treat at least 120 minutes post to the 2C-B HCl form disclosed herein, including those described in Table 3. In some embodiments, the serotonin receptor modulator is olanzapine, wherein the olanzapine is administered to post-treat at least 150 minutes post to the 2C-B HCl form disclosed herein, including those described in Table 3. In some embodiments, the serotonin receptor modulator is olanzapine, wherein the olanzapine is administered to post-treat between about 15 minutes and about 150 minutes post to the 2C-B HCl form disclosed herein, including those described in Table 3. In some embodiments, the serotonin receptor modulator is olanzapine, wherein the olanzapine is administered to post-treat at least 180 minutes post to the 2C-B HCl form disclosed herein, including those described in Table 3. In some embodiments, the serotonin receptor modulator is olanzapine, wherein the olanzapine is administered to post-treat at least 210 minutes post to the 2C-B HCl form disclosed herein, including those described in Table 3.

In some embodiments, the serotonin receptor modulator is olanzapine, wherein the olanzapine is administered to post-treat at least 240 minutes post to the 2C-B HCl form disclosed herein, including those described in Table 3. In some embodiments, the serotonin receptor modulator is olanzapine, wherein the olanzapine is administered to post-treat at least 270 minutes post to the 2C-B HCl form disclosed herein, including those described in Table 3. In some embodiments, the serotonin receptor modulator is olanzapine, wherein the olanzapine is administered to post-treat at least 300 minutes post to the 2C-B HCl form disclosed herein, including those described in Table 3. In some embodiments, the serotonin receptor modulator is olanzapine, wherein the olanzapine is administered to post-treat at least 330 minutes post to the 2C-B HCl form disclosed herein, including those described in Table 3. In some embodiments, the serotonin receptor modulator is olanzapine, wherein the olanzapine is administered to post-treat at least 360 minutes post to the 2C-B HCl form disclosed herein, including those described in Table 3. In some preferred embodiments, the serotonin receptor modulator is olanzapine, wherein olanzapine is administered to post-treat between about 60 minutes and about 180 minutes post to the administration of the 2C-B HCl form disclosed herein, including those described in Table 3.

In some embodiments, the serotonin receptor modulator is quetiapine, wherein the quetiapine is administered to post-treat at least 15 minutes post to the administration of the 2C-B HCl form disclosed herein, including those described in Table 3. In some embodiments, the serotonin receptor modulator is quetiapine, wherein the quetiapine is administered to post-treat at least 30 minutes post to the 2C-B HCl form disclosed herein, including those described in Table 3. In some embodiments, the serotonin receptor modulator is quetiapine, wherein the quetiapine is administered to post-treat between at least 60 minutes and 240 minutes post to the administration or release of the 2C-B HCl form disclosed herein, including those described in Table 3. In some embodiments, the serotonin receptor modulator is quetiapine, wherein the quetiapine is administered to post-treat at least 90 minutes post to the 2C-B HCl form disclosed herein, including those described in Table 3. In some embodiments, the serotonin receptor modulator is quetiapine, wherein the quetiapine is administered to post-treat at least 120 minutes post to the 2C-B HCl form disclosed herein, including those described in Table 3. In some embodiments, the serotonin receptor modulator is quetiapine, wherein the quetiapine is administered to post-treat at least 150 minutes post to the 2C-B HCl form disclosed herein, including those described in Table 3. In some embodiments, the serotonin receptor modulator is quetiapine, wherein the quetiapine is administered to post-treat between about 15 minutes and about 150 minutes post to the 2C-B HCl form disclosed herein, including those described in Table 3. In some embodiments, the serotonin receptor modulator is quetiapine, wherein the quetiapine is administered to post-treat at least 180 minutes post to the 2C-B HCl form disclosed herein, including those described in Table 3. In some embodiments, the serotonin receptor modulator is quetiapine, wherein the quetiapine is administered to post-treat at least 210 minutes post to the 2C-B HCl form disclosed herein, including those described in Table 3.

In some embodiments, the serotonin receptor modulator is quetiapine, wherein the quetiapine is administered to post-treat at least 240 minutes post to the 2C-B HCl form disclosed herein, including those described in Table 3. In some embodiments, the serotonin receptor modulator is quetiapine, wherein the quetiapine is administered to post-treat at least 270 minutes post to the 2C-B HCl form disclosed herein, including those described in Table 3. In some embodiments, the serotonin receptor modulator is quetiapine, wherein the quetiapine is administered to post-treat at least 300 minutes post to the 2C-B HCl form disclosed herein, including those described in Table 3. In some embodiments, the serotonin receptor modulator is quetiapine, wherein the quetiapine is administered to post-treat at least 330 minutes post to the 2C-B HCl form disclosed herein, including those described in Table 3. In some embodiments, the serotonin receptor modulator is quetiapine, wherein the quetiapine is administered to post-treat at least 360 minutes post to the 2C-B HCl form disclosed herein, including those described in Table 3. In some preferred embodiments, the serotonin receptor modulator is quetiapine, wherein quetiapine is administered to post-treat between about 60 minutes and about 180 minutes post to the administration of the 2C-B HCl form disclosed herein, including those described in Table 3.

In some embodiments, the serotonin receptor modulator is risperidone, wherein the risperidone is administered to post-treat at least 15 minutes post to the administration of the 2C-B HCl form disclosed herein, including those described in Table 3. In some embodiments, the serotonin receptor modulator is risperidone, wherein the risperidone is administered to post-treat at least 30 minutes post to the 2C-B HCl form disclosed herein, including those described in Table 3. In some embodiments, the serotonin receptor modulator is risperidone, wherein the risperidone is administered to post-treat between at least 60 minutes and 240 minutes post to the administration or release of the 2C-B HCl form disclosed herein, including those described in Table 3. In some embodiments, the serotonin receptor modulator is risperidone, wherein the risperidone is administered to post-treat at least 90 minutes post to the 2C-B HCl form disclosed herein, including those described in Table 3. In some embodiments, the serotonin receptor modulator is risperidone, wherein the risperidone is administered to post-treat at least 120 minutes post to the 2C-B HCl form disclosed herein, including those described in Table 3. In some embodiments, the serotonin receptor modulator is risperidone, wherein the risperidone is administered to post-treat at least 150 minutes post to the 2C-B HCl form disclosed herein, including those described in Table 3. In some embodiments, the serotonin receptor modulator is risperidone, wherein the risperidone is administered to post-treat between about 15 minutes and about 150 minutes post to the 2C-B HCl form disclosed herein, including those described in Table 3. In some embodiments, the serotonin receptor modulator is risperidone, wherein the risperidone is administered to post-treat at least 180 minutes post to the 2C-B HCl form disclosed herein, including those described in Table 3. In some embodiments, the serotonin receptor modulator is risperidone, wherein the risperidone is administered to post-treat at least 210 minutes post to the 2C-B HCl form disclosed herein, including those described in Table 3.

In some embodiments, the serotonin receptor modulator is risperidone, wherein the risperidone is administered to post-treat at least 240 minutes post to the 2C-B HCl form disclosed herein, including those described in Table 3. In some embodiments, the serotonin receptor modulator is risperidone, wherein the risperidone is administered to post-treat at least 270 minutes post to the 2C-B HCl form disclosed herein, including those described in Table 3. In some embodiments, the serotonin receptor modulator is risperidone, wherein the risperidone is administered to post-treat at least 300 minutes post to the 2C-B HCl form disclosed herein, including those described in Table 3. In some embodiments, the serotonin receptor modulator is risperidone, wherein the risperidone is administered to post-treat at least 330 minutes post to the 2C-B HCl form disclosed herein, including those described in Table 3. In some embodiments, the serotonin receptor modulator is risperidone, wherein the risperidone is administered to post-treat at least 360 minutes post to the 2C-B HCl form disclosed herein, including those described in Table 3. In some preferred embodiments, the serotonin receptor modulator is risperidone, wherein risperidone is administered to post-treat between about 60 minutes and about 180 minutes post to the administration of the 2C-B HCl form disclosed herein, including those described in Table 3.

In some embodiments, the serotonin receptor modulator is eplivanserin and the psychedelic is 2C-B, wherein the eplivanserin is administered to post-treat at least 15 minutes after the administration of the 2C-B form disclosed herein including those described in Table 18. In some embodiments, the serotonin receptor modulator is eplivanserin and the psychedelic is 2C-B, wherein the eplivanserin is administered to post-treat between at least 30 minutes after and 360 minutes after the release or administration of the 2C-B form disclosed herein including those described in Table 18. In some embodiments, the serotonin receptor modulator is eplivanserin and the psychedelic is 2C-B, wherein the eplivanserin is administered to post-treat between at least 60 minutes after and 360 minutes after the release or administration of the 2C-B form disclosed herein including those described in Table 18. In some embodiments, the serotonin receptor modulator is eplivanserin and the psychedelic is 2C-B, wherein the eplivanserin is administered to post-treat at between least 90 minutes and 240 minutes after the 2C-B form disclosed herein including those described in Table 18. In some embodiments, the serotonin receptor modulator is eplivanserin and the psychedelic is 2C-B, wherein the eplivanserin is administered to post-treat at least 120 minutes after the 2C-B form disclosed herein including those described in Table 18.

In some embodiments, the serotonin receptor modulator is eplivanserin and the psychedelic is 2C-B, wherein the eplivanserin is administered to post-treat at least 150 minutes after the 2C-B form disclosed herein including those described in Table 18. In some embodiments, the serotonin receptor modulator is eplivanserin and the psychedelic is 2C-B, wherein the eplivanserin is administered to post-treat between about 15 minutes and about 150 minutes after the 2C-B form disclosed herein including those described in Table 18. In some embodiments, the serotonin receptor modulator is eplivanserin and the psychedelic is 2C-B, wherein the eplivanserin is administered to post-treat at least 180 minutes after the 2C-B form disclosed herein including those described in Table 18. In some embodiments, the serotonin receptor modulator is eplivanserin and the psychedelic is 2C-B, wherein the eplivanserin is administered to post-treat at least 210 minutes after the 2C-B form disclosed herein including those described in Table 18. In some embodiments, the serotonin receptor modulator is eplivanserin and the psychedelic is 2C-B, wherein the eplivanserin is administered to post-treat at least 240 minutes after the 2C-B form disclosed herein including those described in Table 18.

In some embodiments, the serotonin receptor modulator is eplivanserin and the psychedelic is 2C-B, wherein the eplivanserin is administered to post-treat at least 270 minutes after the 2C-B form disclosed herein including those described in Table 18. In some embodiments, the serotonin receptor modulator is eplivanserin and the psychedelic is 2C-B, wherein the eplivanserin is administered to post-treat at least 300 minutes after the 2C-B form disclosed herein including those described in Table 18. In some embodiments, the serotonin receptor modulator is eplivanserin and the psychedelic is 2C-B, wherein the eplivanserin is administered to post-treat at least 330 minutes after the 2C-B form disclosed herein including those described in Table 18. In some embodiments, the serotonin receptor modulator is eplivanserin and the psychedelic is 2C-B, wherein the eplivanserin is administered to post-treat at least 360 minutes after the 2C-B form disclosed herein including those described in Table 18.

In some preferred embodiments, the serotonin receptor modulator is eplivanserin and the psychedelic is 2C-B, wherein eplivanserin is administered to post-treat between about 60 minutes and about 180 minutes after the administration of the 2C-B form disclosed herein including those described in Table 18.

In some embodiments, the serotonin receptor modulator is volinanserin and the psychedelic is 2C-B, wherein the volinanserin is administered to post-treat a subject between at least 15 minutes and 360 minutes after the administration or release of the 2C-B form disclosed herein including those described in Table 18. In some embodiments, the serotonin receptor modulator is volinanserin and the psychedelic is 2C-B, wherein the volinanserin is administered to post-treat between at least 30 minutes and 360 minutes after the administration or release of the 2C-B form disclosed herein including those described in Table 18. In some embodiments, the serotonin receptor modulator is volinanserin and the psychedelic is 2C-B, wherein the volinanserin is administered to post-treat between at least 60 minutes and 240 minutes after the administration or release of the 2C-B form disclosed herein including those described in Table 18. In some embodiments, the serotonin receptor modulator is volinanserin and the psychedelic is 2C-B, wherein the volinanserin is administered to post-treat at least 90 minutes after 2C-B. In some embodiments, the serotonin receptor modulator is volinanserin and the psychedelic is 2C-B, wherein the volinanserin is administered to post-treat at least 120 minutes after the 2C-B form disclosed herein including those described in Table 18.

In some embodiments, the serotonin receptor modulator is volinanserin and the psychedelic is 2C-B, wherein the volinanserin is administered to post-treat at least 150 minutes after the 2C-B form disclosed herein including those described in Table 18. In some embodiments, the serotonin receptor modulator is volinanserin and the psychedelic is 2C-B, wherein the volinanserin is administered to post-treat between about 15 minutes and about 150 minutes after the 2C-B form disclosed herein including those described in Table 18. In some embodiments, the serotonin receptor modulator is volinanserin and the psychedelic is 2C-B, wherein the volinanserin is administered to post-treat at least 180 minutes after the 2C-B form disclosed herein including those described in Table 18. In some embodiments, the serotonin receptor modulator is volinanserin and the psychedelic is 2C-B, wherein the volinanserin is administered to post-treat at least 210 minutes after the 2C-B form disclosed herein including those described in Table 18. In some embodiments, the serotonin receptor modulator is volinanserin and the psychedelic is 2C-B, wherein the volinanserin is administered to post-treat at least 240 minutes after the 2C-B form disclosed herein including those described in Table 18. In some embodiments, the serotonin receptor modulator is volinanserin and the psychedelic is 2C-B, wherein the volinanserin is administered to post-treat at least 270 minutes after the 2C-B form disclosed herein including those described in Table 18. In some embodiments, the serotonin receptor modulator is volinanserin and the psychedelic is 2C-B, wherein the volinanserin is administered to post-treat at least 300 minutes after the 2C-B form disclosed herein including those described in Table 18. In some embodiments, the serotonin receptor modulator is volinanserin and the psychedelic is 2C-B, wherein the volinanserin is administered to post-treat at least 330 minutes after the 2C-B form disclosed herein including those described in Table 18. In some embodiments, the serotonin receptor modulator is volinanserin and the psychedelic is 2C-B, wherein the volinanserin is administered to post-treat at least 360 minutes after the 2C-B form disclosed herein including those described in Table 18. In some preferred embodiments, the serotonin receptor modulator is volinanserin and the psychedelic is 2C-B, wherein volinanserin is administered to post-treat between about 60 minutes and about 180 minutes after the administration of the 2C-B form.

In some embodiments, the serotonin receptor modulator is ketanserin and the psychedelic is 2C-B, wherein the ketanserin is administered to post-treat at least 15 minutes after the administration of the 2C-B form disclosed herein including those described in Table 18. In some embodiments, the serotonin receptor modulator is ketanserin and the psychedelic is 2C-B, wherein the ketanserin is administered to post-treat between at least 30 minutes and 360 minutes after the administration or release of the 2C-B form disclosed herein including those described in Table 18. In some embodiments, the serotonin receptor modulator is ketanserin and the psychedelic is 2C-B, wherein the ketanserin is administered to post-treat between at least 60 minutes and 240 minutes after the administration or release of the 2C-B form disclosed herein including those described in Table 18. In some embodiments, the serotonin receptor modulator is ketanserin and the psychedelic is 2C-B, wherein the ketanserin is administered to post-treat at least 90 minutes after the 2C-B form disclosed herein including those described in Table 18. In some embodiments, the serotonin receptor modulator is ketanserin and the psychedelic is 2C-B, wherein the ketanserin is administered to post-treat at least 120 minutes after the 2C-B form disclosed herein including those described in Table 18. In some embodiments, the serotonin receptor modulator is ketanserin and the psychedelic is 2C-B, wherein the ketanserin is administered to post-treat at least 150 minutes after the 2C-B form disclosed herein including those described in Table 18. In some embodiments, the serotonin receptor modulator is ketanserin and the psychedelic is 2C-B, wherein the ketanserin is administered to post-treat between about 15 minutes and about 150 minutes after the 2C-B form disclosed herein including those described in Table 18. In some embodiments, the serotonin receptor modulator is ketanserin and the psychedelic is 2C-B, wherein the ketanserin is administered to post-treat at least 180 minutes after the 2C-B form disclosed herein including those described in Table 18. In some embodiments, the serotonin receptor modulator is ketanserin and the psychedelic is 2C-B, wherein the ketanserin is administered to post-treat at least 210 minutes after the 2C-B form disclosed herein including those described in Table 18. In some embodiments, the serotonin receptor modulator is ketanserin and the psychedelic is 2C-B, wherein the ketanserin is administered to post-treat at least 240 minutes after the 2C-B form disclosed herein including those described in Table 18. In some embodiments, the serotonin receptor modulator is ketanserin and the psychedelic is 2C-B, wherein the ketanserin is administered to post-treat at least 270 minutes after the 2C-B form disclosed herein including those described in Table 18. In some embodiments, the serotonin receptor modulator is ketanserin and the psychedelic is 2C-B, wherein the ketanserin is administered to post-treat at least 300 minutes after the 2C-B form disclosed herein including those described in Table 18. In some embodiments, the serotonin receptor modulator is ketanserin and the psychedelic is 2C-B, wherein the ketanserin is administered to post-treat at least 330 minutes after the 2C-B form disclosed herein including those described in Table 18. In some embodiments, the serotonin receptor modulator is ketanserin and the psychedelic is 2C-B, wherein the ketanserin is administered to post-treat at least 360 minutes after the 2C-B form disclosed herein including those described in Table 18. In some preferred embodiments, the serotonin receptor modulator is ketanserin and the psychedelic is 2C-B, wherein ketanserin is administered to post-treat between about 60 minutes and about 180 minutes after the administration of the 2C-B form disclosed herein including those described in Table 18.

In some embodiments, the serotonin receptor modulator is ritanserin and the psychedelic is 2C-B, wherein the ritanserin is administered to post-treat at least 15 minutes after the administration of the 2C-B form disclosed herein including those described in Table 18. In some embodiments, the serotonin receptor modulator is ritanserin and the psychedelic is 2C-B, wherein the ritanserin is administered to post-treat at least 30 minutes after the 2C-B form disclosed herein including those described in Table 18. In some embodiments, the serotonin receptor modulator is ritanserin and the psychedelic is 2C-B, wherein the ritanserin is administered to post-treat between at least 60 minutes and 240 minutes after the administration or release of the 2C-B form disclosed herein including those described in Table 18. In some embodiments, the serotonin receptor modulator is ritanserin and the psychedelic is 2C-B, wherein the ritanserin is administered to post-treat at least 90 minutes after the 2C-B form disclosed herein including those described in Table 18. In some embodiments, the serotonin receptor modulator is ritanserin and the psychedelic is 2C-B, wherein the ritanserin is administered to post-treat at least 120 minutes after the 2C-B form disclosed herein including those described in Table 18. In some embodiments, the serotonin receptor modulator is ritanserin and the psychedelic is 2C-B, wherein the ritanserin is administered to post-treat at least 150 minutes after the 2C-B form disclosed herein including those described in Table 18. In some embodiments, the serotonin receptor modulator is ritanserin and the psychedelic is 2C-B, wherein the ritanserin is administered to post-treat between about 15 minutes and about 150 minutes after the 2C-B form disclosed herein including those described in Table 18. In some embodiments, the serotonin receptor modulator is ritanserin and the psychedelic is 2C-B, wherein the ritanserin is administered to post-treat at least 180 minutes after the 2C-B form disclosed herein including those described in Table 18. In some embodiments, the serotonin receptor modulator is ritanserin and the psychedelic is 2C-B, wherein the ritanserin is administered to post-treat at least 210 minutes after the 2C-B form disclosed herein including those described in Table 18. In some embodiments, the serotonin receptor modulator is ritanserin and the psychedelic is 2C-B, wherein the ritanserin is administered to post-treat at least 240 minutes after the 2C-B form disclosed herein including those described in Table 18. In some embodiments, the serotonin receptor modulator is ritanserin and the psychedelic is 2C-B, wherein the ritanserin is administered to post-treat at least 270 minutes after the 2C-B form disclosed herein including those described in Table 18. In some embodiments, the serotonin receptor modulator is ritanserin and the psychedelic is 2C-B, wherein the ritanserin is administered to post-treat at least 300 minutes after the 2C-B form disclosed herein including those described in Table 18. In some embodiments, the serotonin receptor modulator is ritanserin and the psychedelic is 2C-B, wherein the ritanserin is administered to post-treat at least 330 minutes after the 2C-B form disclosed herein including those described in Table 18. In some embodiments, the serotonin receptor modulator is ritanserin and the psychedelic is 2C-B, wherein the ritanserin is administered to post-treat at least 360 minutes after the 2C-B form disclosed herein including those described in Table 18. In some preferred embodiments, the serotonin receptor modulator is ritanserin and the psychedelic is 2C-B, wherein ritanserin is administered to post-treat between about 60 minutes and about 180 minutes after the administration of the 2C-B form disclosed herein including those described in Table 18.

In some embodiments, the serotonin receptor modulator is pimavanserin and the psychedelic is 2C-B, wherein the pimavanserin is administered to post-treat at least 15 minutes after the administration of the 2C-B form disclosed herein including those described in Table 18. In some embodiments, the serotonin receptor modulator is pimavanserin and the psychedelic is 2C-B, wherein the pimavanserin is administered to post-treat at least 30 minutes after the 2C-B form disclosed herein including those described in Table 18. In some embodiments, the serotonin receptor modulator is pimavanserin and the psychedelic is 2C-B, wherein the pimavanserin is administered to post-treat between at least 60 minutes and 240 minutes after the administration or release of the 2C-B form disclosed herein including those described in Table 18. In some embodiments, the serotonin receptor modulator is pimavanserin and the psychedelic is 2C-B, wherein the pimavanserin is administered to post-treat at least 90 minutes after the 2C-B form disclosed herein including those described in Table 18. In some embodiments, the serotonin receptor modulator is pimavanserin and the psychedelic is 2C-B, wherein the pimavanserin is administered to post-treat at least 120 minutes after the 2C-B form disclosed herein including those described in Table 18. In some embodiments, the serotonin receptor modulator is pimavanserin and the psychedelic is 2C-B, wherein the pimavanserin is administered to post-treat at least 150 minutes after the 2C-B form disclosed herein including those described in Table 18. In some embodiments, the serotonin receptor modulator is pimavanserin and the psychedelic is 2C-B, wherein the pimavanserin is administered to post-treat between about 15 minutes and about 150 minutes after the 2C-B form disclosed herein including those described in Table 18. In some embodiments, the serotonin receptor modulator is pimavanserin and the psychedelic is 2C-B, wherein the pimavanserin is administered to post-treat at least 180 minutes after the 2C-B form disclosed herein including those described in Table 18. In some embodiments, the serotonin receptor modulator is pimavanserin and the psychedelic is 2C-B, wherein the pimavanserin is administered to post-treat at least 210 minutes after the 2C-B form disclosed herein including those described in Table 18. In some embodiments, the serotonin receptor modulator is pimavanserin and the psychedelic is 2C-B, wherein the pimavanserin is administered to post-treat at least 240 minutes after the 2C-B form disclosed herein including those described in Table 18. In some embodiments, the serotonin receptor modulator is pimavanserin and the psychedelic is 2C-B, wherein the pimavanserin is administered to post-treat at least 270 minutes after the 2C-B form disclosed herein including those described in Table 18. In some embodiments, the serotonin receptor modulator is pimavanserin and the psychedelic is 2C-B, wherein the pimavanserin is administered to post-treat at least 300 minutes after the 2C-B form disclosed herein including those described in Table 18. In some embodiments, the serotonin receptor modulator is pimavanserin and the psychedelic is 2C-B, wherein the pimavanserin is administered to post-treat at least 330 minutes after the 2C-B form disclosed herein including those described in Table 18. In some embodiments, the serotonin receptor modulator is pimavanserin and the psychedelic is 2C-B, wherein the pimavanserin is administered to post-treat at least 360 minutes after the 2C-B form disclosed herein including those described in Table 18. In some preferred embodiments, the serotonin receptor modulator is pimavanserin and the psychedelic is 2C-B, wherein pimavanserin is administered to post-treat between about 60 minutes and about 180 minutes after the administration of the 2C-B form disclosed herein including those described in Table 18.

In some embodiments, the serotonin receptor modulator is nelotanserin and the psychedelic is 2C-B, wherein the nelotanserin is administered to post-treat at least 15 minutes after the administration of the 2C-B form disclosed herein including those described in Table 18. In some embodiments, the serotonin receptor modulator is nelotanserin and the psychedelic is 2C-B, wherein the nelotanserin is administered to post-treat at least 30 minutes after the 2C-B form disclosed herein including those described in Table 18. In some embodiments, the serotonin receptor modulator is nelotanserin and the psychedelic is 2C-B, wherein the nelotanserin is administered to post-treat between at least 60 minutes and 240 minutes after the administration or release of the 2C-B form disclosed herein including those described in Table 18. In some embodiments, the serotonin receptor modulator is nelotanserin and the psychedelic is 2C-B, wherein the nelotanserin is administered to post-treat at least 90 minutes after the 2C-B form disclosed herein including those described in Table 18. In some embodiments, the serotonin receptor modulator is nelotanserin and the psychedelic is 2C-B, wherein the nelotanserin is administered to post-treat at least 120 minutes after the 2C-B form disclosed herein including those described in Table 18.

In some embodiments, the serotonin receptor modulator is nelotanserin and the psychedelic is 2C-B, wherein the nelotanserin is administered to post-treat at least 150 minutes after the 2C-B form disclosed herein including those described in Table 18. In some embodiments, the serotonin receptor modulator is nelotanserin and the psychedelic is 2C-B, wherein the nelotanserin is administered to post-treat between about 15 minutes and about 150 minutes after the 2C-B form disclosed herein including those described in Table 18. In some embodiments, the serotonin receptor modulator is nelotanserin and the psychedelic is 2C-B, wherein the nelotanserin is administered to post-treat at least 180 minutes after the 2C-B form disclosed herein including those described in Table 18. In some embodiments, the serotonin receptor modulator is nelotanserin and the psychedelic is 2C-B, wherein the nelotanserin is administered to post-treat at least 210 minutes after the 2C-B form disclosed herein including those described in Table 18. In some embodiments, the serotonin receptor modulator is nelotanserin and the psychedelic is 2C-B, wherein the nelotanserin is administered to post-treat at least 240 minutes after the 2C-B form disclosed herein including those described in Table 18. In some embodiments, the serotonin receptor modulator is nelotanserin and the psychedelic is 2C-B, wherein the nelotanserin is administered to post-treat at least 270 minutes after the 2C-B form disclosed herein including those described in Table 18.

In some embodiments, the serotonin receptor modulator is nelotanserin and the psychedelic is 2C-B, wherein the nelotanserin is administered to post-treat at least 300 minutes after the 2C-B form disclosed herein including those described in Table 18. In some embodiments, the serotonin receptor modulator is nelotanserin and the psychedelic is 2C-B, wherein the nelotanserin is administered to post-treat at least 330 minutes after the 2C-B form disclosed herein including those described in Table 18. In some embodiments, the serotonin receptor modulator is nelotanserin and the psychedelic is 2C-B, wherein the nelotanserin is administered to post-treat at least 360 minutes after the 2C-B form disclosed herein including those described in Table 18. In some preferred embodiments, the serotonin receptor modulator is nelotanserin and the psychedelic is 2C-B, wherein nelotanserin is administered to post-treat between about 60 minutes and about 180 minutes after the administration of the 2C-B form disclosed herein including those described in Table 18.

In some embodiments, the serotonin receptor modulator is pruvanserin and the psychedelic is 2C-B, wherein the pruvanserin is administered to post-treat at least 15 minutes after the administration of the 2C-B form disclosed herein including those described in Table 18. In some embodiments, the serotonin receptor modulator is pruvanserin and the psychedelic is 2C-B, wherein the pruvanserin is administered to post-treat at least 30 minutes after the 2C-B form disclosed herein including those described in Table 18. In some embodiments, the serotonin receptor modulator is pruvanserin and the psychedelic is 2C-B, wherein the pruvanserin is administered to post-treat between at least 60 minutes and 240 minutes after the administration or release of the 2C-B form disclosed herein including those described in Table 18. In some embodiments, the serotonin receptor modulator is pruvanserin and the psychedelic is 2C-B, wherein the pruvanserin is administered to post-treat at least 90 minutes after the 2C-B form disclosed herein including those described in Table 18. In some embodiments, the serotonin receptor modulator is pruvanserin and the psychedelic is 2C-B, wherein the pruvanserin is administered to post-treat at least 120 minutes after the 2C-B form disclosed herein including those described in Table 18. In some embodiments, the serotonin receptor modulator is pruvanserin and the psychedelic is 2C-B, wherein the pruvanserin is administered to post-treat at least 150 minutes after the 2C-B form disclosed herein including those described in Table 18. In some embodiments, the serotonin receptor modulator is pruvanserin and the psychedelic is 2C-B, wherein the pruvanserin is administered to post-treat between about 15 minutes and about 150 minutes after the 2C-B form disclosed herein including those described in Table 18. In some embodiments, the serotonin receptor modulator is pruvanserin and the psychedelic is 2C-B, wherein the pruvanserin is administered to post-treat at least 180 minutes after the 2C-B form disclosed herein including those described in Table 18. In some embodiments, the serotonin receptor modulator is pruvanserin and the psychedelic is 2C-B, wherein the pruvanserin is administered to post-treat at least 210 minutes after the 2C-B form disclosed herein including those described in Table 18.

In some embodiments, the serotonin receptor modulator is pruvanserin and the psychedelic is 2C-B, wherein the pruvanserin is administered to post-treat at least 240 minutes after the 2C-B form disclosed herein including those described in Table 18. In some embodiments, the serotonin receptor modulator is pruvanserin and the psychedelic is 2C-B, wherein the pruvanserin is administered to post-treat at least 270 minutes after the 2C-B form disclosed herein including those described in Table 18. In some embodiments, the serotonin receptor modulator is pruvanserin and the psychedelic is 2C-B, wherein the pruvanserin is administered to post-treat at least 300 minutes after the 2C-B form disclosed herein including those described in Table 18. In some embodiments, the serotonin receptor modulator is pruvanserin and the psychedelic is 2C-B, wherein the pruvanserin is administered to post-treat at least 330 minutes after the 2C-B form disclosed herein including those described in Table 18. In some embodiments, the serotonin receptor modulator is pruvanserin and the psychedelic is 2C-B, wherein the pruvanserin is administered to post-treat at least 360 minutes after the 2C-B form disclosed herein including those described in Table 18. In some preferred embodiments, the serotonin receptor modulator is pruvanserin and the psychedelic is 2C-B, wherein pruvanserin is administered to post-treat between about 60 minutes and about 180 minutes after the administration of the 2C-B form disclosed herein including those described in Table 18.

In some embodiments, the serotonin receptor modulator is flibanserin, wherein the flibanserin is administered to post-treat at least 15 minutes post to the administration of the 2C-B form disclosed herein including those described in Table 18. In some embodiments, the serotonin receptor modulator is flibanserin, wherein the flibanserin is administered to post-treat at least 30 minutes post to the 2C-B form disclosed herein including those described in Table 18. In some embodiments, the serotonin receptor modulator is flibanserin, wherein the flibanserin is administered to post-treat between at least 60 minutes and 240 minutes post to the administration or release of the 2C-B form disclosed herein including those described in Table 18. In some embodiments, the serotonin receptor modulator is flibanserin, wherein the flibanserin is administered to post-treat at least 90 minutes post to the 2C-B form disclosed herein including those described in Table 18. In some embodiments, the serotonin receptor modulator is flibanserin, wherein the flibanserin is administered to post-treat at least 120 minutes post to the 2C-B form disclosed herein including those described in Table 18. In some embodiments, the serotonin receptor modulator is flibanserin, wherein the flibanserin is administered to post-treat at least 150 minutes post to the 2C-B form disclosed herein including those described in Table 18. In some embodiments, the serotonin receptor modulator is flibanserin, wherein the flibanserin is administered to post-treat between about 15 minutes and about 150 minutes post to the 2C-B form disclosed herein including those described in Table 18. In some embodiments, the serotonin receptor modulator is flibanserin, wherein the flibanserin is administered to post-treat at least 180 minutes post to the 2C-B form disclosed herein including those described in Table 18. In some embodiments, the serotonin receptor modulator is flibanserin, wherein the flibanserin is administered to post-treat at least 210 minutes post to the 2C-B form disclosed herein including those described in Table 18.

In some embodiments, the serotonin receptor modulator is flibanserin, wherein the flibanserin is administered to post-treat at least 240 minutes post to the 2C-B form disclosed herein including those described in Table 18. In some embodiments, the serotonin receptor modulator is flibanserin, wherein the flibanserin is administered to post-treat at least 270 minutes post to the 2C-B form disclosed herein including those described in Table 18. In some embodiments, the serotonin receptor modulator is flibanserin, wherein the flibanserin is administered to post-treat at least 300 minutes post to the 2C-B form disclosed herein including those described in Table 18. In some embodiments, the serotonin receptor modulator is flibanserin, wherein the flibanserin is administered to post-treat at least 330 minutes post to the 2C-B form disclosed herein including those described in Table 18. In some embodiments, the serotonin receptor modulator is flibanserin, wherein the flibanserin is administered to post-treat at least 360 minutes post to the 2C-B form disclosed herein including those described in Table 18. In some preferred embodiments, the serotonin receptor modulator is flibanserin, wherein flibanserin is administered to post-treat between about 60 minutes and about 180 minutes post to the administration of the 2C-B form disclosed herein including those described in Table 18.

In some embodiments, the serotonin receptor modulator is olanzapine, wherein the olanzapine is administered to post-treat at least 15 minutes post to the administration of the 2C-B form disclosed herein including those described in Table 18. In some embodiments, the serotonin receptor modulator is olanzapine, wherein the olanzapine is administered to post-treat at least 30 minutes post to the 2C-B form disclosed herein including those described in Table 18. In some embodiments, the serotonin receptor modulator is olanzapine, wherein the olanzapine is administered to post-treat between at least 60 minutes and 240 minutes post to the administration or release of the 2C-B form disclosed herein including those described in Table 18. In some embodiments, the serotonin receptor modulator is olanzapine, wherein the olanzapine is administered to post-treat at least 90 minutes post to the 2C-B form disclosed herein including those described in Table 18. In some embodiments, the serotonin receptor modulator is olanzapine, wherein the olanzapine is administered to post-treat at least 120 minutes post to the 2C-B form disclosed herein including those described in Table 18. In some embodiments, the serotonin receptor modulator is olanzapine, wherein the olanzapine is administered to post-treat at least 150 minutes post to the 2C-B form disclosed herein including those described in Table 18. In some embodiments, the serotonin receptor modulator is olanzapine, wherein the olanzapine is administered to post-treat between about 15 minutes and about 150 minutes post to the 2C-B form disclosed herein including those described in Table 18. In some embodiments, the serotonin receptor modulator is olanzapine, wherein the olanzapine is administered to post-treat at least 180 minutes post to the 2C-B form disclosed herein including those described in Table 18. In some embodiments, the serotonin receptor modulator is olanzapine, wherein the olanzapine is administered to post-treat at least 210 minutes post to the 2C-B form disclosed herein including those described in Table 18.

In some embodiments, the serotonin receptor modulator is olanzapine, wherein the olanzapine is administered to post-treat at least 240 minutes post to the 2C-B form disclosed herein including those described in Table 18. In some embodiments, the serotonin receptor modulator is olanzapine, wherein the olanzapine is administered to post-treat at least 270 minutes post to the 2C-B form disclosed herein including those described in Table 18. In some embodiments, the serotonin receptor modulator is olanzapine, wherein the olanzapine is administered to post-treat at least 300 minutes post to the 2C-B form disclosed herein including those described in Table 18. In some embodiments, the serotonin receptor modulator is olanzapine, wherein the olanzapine is administered to post-treat at least 330 minutes post to the 2C-B form disclosed herein including those described in Table 18. In some embodiments, the serotonin receptor modulator is olanzapine, wherein the olanzapine is administered to post-treat at least 360 minutes post to the 2C-B form disclosed herein including those described in Table 18. In some preferred embodiments, the serotonin receptor modulator is olanzapine, wherein olanzapine is administered to post-treat between about 60 minutes and about 180 minutes post to the administration of the 2C-B form disclosed herein including those described in Table 18.

In some embodiments, the serotonin receptor modulator is quetiapine, wherein the quetiapine is administered to post-treat at least 15 minutes post to the administration of the 2C-B form disclosed herein including those described in Table 18. In some embodiments, the serotonin receptor modulator is quetiapine, wherein the quetiapine is administered to post-treat at least 30 minutes post to the 2C-B form disclosed herein including those described in Table 18. In some embodiments, the serotonin receptor modulator is quetiapine, wherein the quetiapine is administered to post-treat between at least 60 minutes and 240 minutes post to the administration or release of the 2C-B form disclosed herein including those described in Table 18. In some embodiments, the serotonin receptor modulator is quetiapine, wherein the quetiapine is administered to post-treat at least 90 minutes post to the 2C-B form disclosed herein including those described in Table 18. In some embodiments, the serotonin receptor modulator is quetiapine, wherein the quetiapine is administered to post-treat at least 120 minutes post to the 2C-B form disclosed herein including those described in Table 18. In some embodiments, the serotonin receptor modulator is quetiapine, wherein the quetiapine is administered to post-treat at least 150 minutes post to the 2C-B form disclosed herein including those described in Table 18. In some embodiments, the serotonin receptor modulator is quetiapine, wherein the quetiapine is administered to post-treat between about 15 minutes and about 150 minutes post to the 2C-B form disclosed herein including those described in Table 18. In some embodiments, the serotonin receptor modulator is quetiapine, wherein the quetiapine is administered to post-treat at least 180 minutes post to the 2C-B form disclosed herein including those described in Table 18. In some embodiments, the serotonin receptor modulator is quetiapine, wherein the quetiapine is administered to post-treat at least 210 minutes post to the 2C-B form disclosed herein including those described in Table 18.

In some embodiments, the serotonin receptor modulator is quetiapine, wherein the quetiapine is administered to post-treat at least 240 minutes post to the 2C-B form disclosed herein including those described in Table 18. In some embodiments, the serotonin receptor modulator is quetiapine, wherein the quetiapine is administered to post-treat at least 270 minutes post to the 2C-B form disclosed herein including those described in Table 18. In some embodiments, the serotonin receptor modulator is quetiapine, wherein the quetiapine is administered to post-treat at least 300 minutes post to the 2C-B form disclosed herein including those described in Table 18. In some embodiments, the serotonin receptor modulator is quetiapine, wherein the quetiapine is administered to post-treat at least 330 minutes post to the 2C-B form disclosed herein including those described in Table 18. In some embodiments, the serotonin receptor modulator is quetiapine, wherein the quetiapine is administered to post-treat at least 360 minutes post to the 2C-B form disclosed herein including those described in Table 18. In some preferred embodiments, the serotonin receptor modulator is quetiapine, wherein quetiapine is administered to post-treat between about 60 minutes and about 180 minutes post to the administration of the 2C-B form disclosed herein including those described in Table 18.

In some embodiments, the serotonin receptor modulator is risperidone, wherein the risperidone is administered to post-treat at least 15 minutes post to the administration of the 2C-B form disclosed herein including those described in Table 18. In some embodiments, the serotonin receptor modulator is risperidone, wherein the risperidone is administered to post-treat at least 30 minutes post to the 2C-B form disclosed herein including those described in Table 18. In some embodiments, the serotonin receptor modulator is risperidone, wherein the risperidone is administered to post-treat between at least 60 minutes and 240 minutes post to the administration or release of the 2C-B form disclosed herein including those described in Table 18. In some embodiments, the serotonin receptor modulator is risperidone, wherein the risperidone is administered to post-treat at least 90 minutes post to the 2C-B form disclosed herein including those described in Table 18. In some embodiments, the serotonin receptor modulator is risperidone, wherein the risperidone is administered to post-treat at least 120 minutes post to the 2C-B form disclosed herein including those described in Table 18. In some embodiments, the serotonin receptor modulator is risperidone, wherein the risperidone is administered to post-treat at least 150 minutes post to the 2C-B form disclosed herein including those described in Table 18. In some embodiments, the serotonin receptor modulator is risperidone, wherein the risperidone is administered to post-treat between about 15 minutes and about 150 minutes post to the 2C-B form disclosed herein including those described in Table 18. In some embodiments, the serotonin receptor modulator is risperidone, wherein the risperidone is administered to post-treat at least 180 minutes post to the 2C-B form disclosed herein including those described in Table 18. In some embodiments, the serotonin receptor modulator is risperidone, wherein the risperidone is administered to post-treat at least 210 minutes post to the 2C-B form disclosed herein including those described in Table 18.

In some embodiments, the serotonin receptor modulator is risperidone, wherein the risperidone is administered to post-treat at least 240 minutes post to the 2C-B form disclosed herein including those described in Table 18. In some embodiments, the serotonin receptor modulator is risperidone, wherein the risperidone is administered to post-treat at least 270 minutes post to the 2C-B form disclosed herein including those described in Table 18. In some embodiments, the serotonin receptor modulator is risperidone, wherein the risperidone is administered to post-treat at least 300 minutes post to the 2C-B form disclosed herein including those described in Table 18. In some embodiments, the serotonin receptor modulator is risperidone, wherein the risperidone is administered to post-treat at least 330 minutes post to the 2C-B form disclosed herein including those described in Table 18. In some embodiments, the serotonin receptor modulator is risperidone, wherein the risperidone is administered to post-treat at least 360 minutes post to the 2C-B form disclosed herein including those described in Table 18. In some preferred embodiments, the serotonin receptor modulator is risperidone, wherein risperidone is administered to post-treat between about 60 minutes and about 180 minutes post to the administration of the 2C-B form disclosed herein including those described in Table 18.

In some embodiments, the serotonin receptor modulator is eplivanserin and the psychedelic is 2C-B tartrate Form 1, wherein the eplivanserin is administered to post-treat at least 15 minutes after the administration of the 2C-B tartrate Form 1. In some embodiments, the serotonin receptor modulator is eplivanserin and the psychedelic is 2C-B tartrate Form 1, wherein the eplivanserin is administered to post-treat between at least 30 minutes after and 360 minutes after the release or administration of the 2C-B tartrate Form 1. In some embodiments, the serotonin receptor modulator is eplivanserin and the psychedelic is 2C-B tartrate Form 1, wherein the eplivanserin is administered to post-treat between at least 60 minutes after and 360 minutes after the release or administration of the 2C-B tartrate Form 1. In some embodiments, the serotonin receptor modulator is eplivanserin and the psychedelic is 2C-B tartrate Form 1, wherein the eplivanserin is administered to post-treat between at least 90 minutes and 240 minutes after the 2C-B tartrate Form 1. In some embodiments, the serotonin receptor modulator is eplivanserin and the psychedelic is 2C-B tartrate Form 1, wherein the eplivanserin is administered to post-treat at least 120 minutes after the 2C-B tartrate Form 1.

In some embodiments, the serotonin receptor modulator is eplivanserin and the psychedelic is 2C-B tartrate Form 1, wherein the eplivanserin is administered to post-treat at least 150 minutes after the 2C-B tartrate Form 1. In some embodiments, the serotonin receptor modulator is eplivanserin and the psychedelic is 2C-B tartrate Form 1, wherein the eplivanserin is administered to post-treat between about 15 minutes and about 150 minutes after the 2C-B tartrate Form 1. In some embodiments, the serotonin receptor modulator is eplivanserin and the psychedelic is 2C-B tartrate Form 1, wherein the eplivanserin is administered to post-treat at least 180 minutes after the 2C-B tartrate Form 1. In some embodiments, the serotonin receptor modulator is eplivanserin and the psychedelic is 2C-B tartrate Form 1, wherein the eplivanserin is administered to post-treat at least 210 minutes after the 2C-B tartrate Form 1. In some embodiments, the serotonin receptor modulator is eplivanserin and the psychedelic is 2C-B tartrate Form 1, wherein the eplivanserin is administered to post-treat at least 240 minutes after the 2C-B tartrate Form 1.

In some embodiments, the serotonin receptor modulator is eplivanserin and the psychedelic is 2C-B tartrate Form 1, wherein the eplivanserin is administered to post-treat at least 270 minutes after the 2C-B tartrate Form 1. In some embodiments, the serotonin receptor modulator is eplivanserin and the psychedelic is 2C-B tartrate Form 1, wherein the eplivanserin is administered to post-treat at least 300 minutes after the 2C-B tartrate Form 1. In some embodiments, the serotonin receptor modulator is eplivanserin and the psychedelic is 2C-B tartrate Form 1, wherein the eplivanserin is administered to post-treat at least 330 minutes after the 2C-B tartrate Form 1. In some embodiments, the serotonin receptor modulator is eplivanserin and the psychedelic is 2C-B tartrate Form 1, wherein the eplivanserin is administered to post-treat at least 360 minutes after the 2C-B tartrate Form 1.

In some preferred embodiments, the serotonin receptor modulator is eplivanserin and the psychedelic is 2C-B tartrate Form 1, wherein eplivanserin is administered to post-treat between about 60 minutes and about 180 minutes after the administration of the 2C-B tartrate Form 1. In some embodiments, the serotonin receptor modulator is volinanserin and the psychedelic is 2C-B tartrate Form 1, wherein the volinanserin is administered to post-treat a subject between at least 15 minutes and 360 minutes after the administration or release of the 2C-B tartrate Form 1. In some embodiments, the serotonin receptor modulator is volinanserin and the psychedelic is 2C-B tartrate Form 1, wherein the volinanserin is administered to post-treat between at least 30 minutes and 360 minutes after the administration or release of the 2C-B tartrate Form 1. In some embodiments, the serotonin receptor modulator is volinanserin and the psychedelic is 2C-B tartrate Form 1, wherein the volinanserin is administered to post-treat between at least 60 minutes and 240 minutes after the administration or release of the 2C-B tartrate Form 1. In some embodiments, the serotonin receptor modulator is volinanserin and the psychedelic is 2C-B tartrate Form 1, wherein the volinanserin is administered to post-treat at least 90 minutes after 2C-B tartrate Form 1. In some embodiments, the serotonin receptor modulator is volinanserin and the psychedelic is 2C-B tartrate Form 1, wherein the volinanserin is administered to post-treat at least 120 minutes after the 2C-B tartrate Form 1.

In some embodiments, the serotonin receptor modulator is volinanserin and the psychedelic is 2C-B tartrate Form 1, wherein the volinanserin is administered to post-treat at least 150 minutes after the 2C-B tartrate Form 1. In some embodiments, the serotonin receptor modulator is volinanserin and the psychedelic is 2C-B tartrate Form 1, wherein the volinanserin is administered to post-treat between about 15 minutes and about 150 minutes after the 2C-B tartrate Form 1. In some embodiments, the serotonin receptor modulator is volinanserin and the psychedelic is 2C-B tartrate Form 1, wherein the volinanserin is administered to post-treat at least 180 minutes after the 2C-B tartrate Form 1. In some embodiments, the serotonin receptor modulator is volinanserin and the psychedelic is 2C-B tartrate Form 1, wherein the volinanserin is administered to post-treat at least 210 minutes after the 2C-B tartrate Form 1. In some embodiments, the serotonin receptor modulator is volinanserin and the psychedelic is 2C-B tartrate Form 1, wherein the volinanserin is administered to post-treat at least 240 minutes after the 2C-B tartrate Form 1. In some embodiments, the serotonin receptor modulator is volinanserin and the psychedelic is 2C-B tartrate Form 1, wherein the volinanserin is administered to post-treat at least 270 minutes after the 2C-B tartrate Form 1. In some embodiments, the serotonin receptor modulator is volinanserin and the psychedelic is 2C-B tartrate Form 1, wherein the volinanserin is administered to post-treat at least 300 minutes after the 2C-B tartrate Form 1. In some embodiments, the serotonin receptor modulator is volinanserin and the psychedelic is 2C-B tartrate Form 1, wherein the volinanserin is administered to post-treat at least 330 minutes after the 2C-B tartrate Form 1. In some embodiments, the serotonin receptor modulator is volinanserin and the psychedelic is 2C-B tartrate Form 1, wherein the volinanserin is administered to post-treat at least 360 minutes after the 2C-B tartrate Form 1. In some preferred embodiments, the serotonin receptor modulator is volinanserin and the psychedelic is 2C-B tartrate Form 1, wherein volinanserin is administered to post-treat between about 60 minutes and about 180 minutes after the administration of the 2C-B tartrate Form 1.

In some embodiments, the serotonin receptor modulator is ketanserin and the psychedelic is 2C-B tartrate Form 1, wherein the ketanserin is administered to post-treat at least 15 minutes after the administration of the 2C-B tartrate Form 1. In some embodiments, the serotonin receptor modulator is ketanserin and the psychedelic is 2C-B tartrate Form 1, wherein the ketanserin is administered to post-treat between at least 30 minutes and 360 minutes after the administration or release of the 2C-B tartrate Form 1. In some embodiments, the serotonin receptor modulator is ketanserin and the psychedelic is 2C-B tartrate Form 1, wherein the ketanserin is administered to post-treat between at least 60 minutes and 240 minutes after the administration or release of the 2C-B tartrate Form 1. In some embodiments, the serotonin receptor modulator is ketanserin and the psychedelic is 2C-B tartrate Form 1, wherein the ketanserin is administered to post-treat at least 90 minutes after the 2C-B tartrate Form 1. In some embodiments, the serotonin receptor modulator is ketanserin and the psychedelic is 2C-B tartrate Form 1, wherein the ketanserin is administered to post-treat at least 120 minutes after the 2C-B tartrate Form 1. In some embodiments, the serotonin receptor modulator is ketanserin and the psychedelic is 2C-B tartrate Form 1, wherein the ketanserin is administered to post-treat at least 150 minutes after the 2C-B tartrate Form 1. In some embodiments, the serotonin receptor modulator is ketanserin and the psychedelic is 2C-B tartrate Form 1, wherein the ketanserin is administered to post-treat between about 15 minutes and about 150 minutes after the 2C-B tartrate Form 1. In some embodiments, the serotonin receptor modulator is ketanserin and the psychedelic is 2C-B tartrate Form 1, wherein the ketanserin is administered to post-treat at least 180 minutes after the 2C-B tartrate Form 1. In some embodiments, the serotonin receptor modulator is ketanserin and the psychedelic is 2C-B tartrate Form 1, wherein the ketanserin is administered to post-treat at least 210 minutes after the 2C-B tartrate Form 1. In some embodiments, the serotonin receptor modulator is ketanserin and the psychedelic is 2C-B tartrate Form 1, wherein the ketanserin is administered to post-treat at least 240 minutes after the 2C-B tartrate Form 1. In some embodiments, the serotonin receptor modulator is ketanserin and the psychedelic is 2C-B tartrate Form 1, wherein the ketanserin is administered to post-treat at least 270 minutes after the 2C-B tartrate Form 1. In some embodiments, the serotonin receptor modulator is ketanserin and the psychedelic is 2C-B tartrate Form 1, wherein the ketanserin is administered to post-treat at least 300 minutes after the 2C-B tartrate Form 1. In some embodiments, the serotonin receptor modulator is ketanserin and the psychedelic is 2C-B tartrate Form 1, wherein the ketanserin is administered to post-treat at least 330 minutes after the 2C-B tartrate Form 1. In some embodiments, the serotonin receptor modulator is ketanserin and the psychedelic is 2C-B tartrate Form 1, wherein the ketanserin is administered to post-treat at least 360 minutes after the 2C-B tartrate Form 1. In some preferred embodiments, the serotonin receptor modulator is ketanserin and the psychedelic is 2C-B tartrate Form 1, wherein ketanserin is administered to post-treat between about 60 minutes and about 180 minutes after the administration of the 2C-B tartrate Form 1.

In some embodiments, the serotonin receptor modulator is ritanserin and the psychedelic is 2C-B tartrate Form 1, wherein the ritanserin is administered to post-treat at least 15 minutes after the administration of the 2C-B tartrate Form 1. In some embodiments, the serotonin receptor modulator is ritanserin and the psychedelic is 2C-B tartrate Form 1, wherein the ritanserin is administered to post-treat at least 30 minutes after the 2C-B tartrate Form 1. In some embodiments, the serotonin receptor modulator is ritanserin and the psychedelic is 2C-B tartrate Form 1, wherein the ritanserin is administered to post-treat between at least 60 minutes and 240 minutes after the administration or release of the 2C-B tartrate Form 1. In some embodiments, the serotonin receptor modulator is ritanserin and the psychedelic is 2C-B tartrate Form 1, wherein the ritanserin is administered to post-treat at least 90 minutes after the 2C-B tartrate Form 1. In some embodiments, the serotonin receptor modulator is ritanserin and the psychedelic is 2C-B tartrate Form 1, wherein the ritanserin is administered to post-treat at least 120 minutes after the 2C-B tartrate Form 1. In some embodiments, the serotonin receptor modulator is ritanserin and the psychedelic is 2C-B tartrate Form 1, wherein the ritanserin is administered to post-treat at least 150 minutes after the 2C-B tartrate Form 1. In some embodiments, the serotonin receptor modulator is ritanserin and the psychedelic is 2C-B tartrate Form 1, wherein the ritanserin is administered to post-treat between about 15 minutes and about 150 minutes after the 2C-B tartrate Form 1. In some embodiments, the serotonin receptor modulator is ritanserin and the psychedelic is 2C-B tartrate Form 1, wherein the ritanserin is administered to post-treat at least 180 minutes after the 2C-B tartrate Form 1. In some embodiments, the serotonin receptor modulator is ritanserin and the psychedelic is 2C-B tartrate Form 1, wherein the ritanserin is administered to post-treat at least 210 minutes after the 2C-B tartrate Form 1. In some embodiments, the serotonin receptor modulator is ritanserin and the psychedelic is 2C-B tartrate Form 1, wherein the ritanserin is administered to post-treat at least 240 minutes after the 2C-B tartrate Form 1. In some embodiments, the serotonin receptor modulator is ritanserin and the psychedelic is 2C-B tartrate Form 1, wherein the ritanserin is administered to post-treat at least 270 minutes after the 2C-B tartrate Form 1. In some embodiments, the serotonin receptor modulator is ritanserin and the psychedelic is 2C-B tartrate Form 1, wherein the ritanserin is administered to post-treat at least 300 minutes after the 2C-B tartrate Form 1. In some embodiments, the serotonin receptor modulator is ritanserin and the psychedelic is 2C-B tartrate Form 1, wherein the ritanserin is administered to post-treat at least 330 minutes after the 2C-B tartrate Form 1. In some embodiments, the serotonin receptor modulator is ritanserin and the psychedelic is 2C-B tartrate Form 1, wherein the ritanserin is administered to post-treat at least 360 minutes after the 2C-B tartrate Form 1. In some preferred embodiments, the serotonin receptor modulator is ritanserin and the psychedelic is 2C-B tartrate Form 1, wherein ritanserin is administered to post-treat between about 60 minutes and about 180 minutes after the administration of the 2C-B tartrate Form 1.

In some embodiments, the serotonin receptor modulator is pimavanserin and the psychedelic is 2C-B tartrate Form 1, wherein the pimavanserin is administered to post-treat at least 15 minutes after the administration of the 2C-B tartrate Form 1. In some embodiments, the serotonin receptor modulator is pimavanserin and the psychedelic is 2C-B tartrate Form 1, wherein the pimavanserin is administered to post-treat at least 30 minutes after the 2C-B tartrate Form 1. In some embodiments, the serotonin receptor modulator is pimavanserin and the psychedelic is 2C-B tartrate Form 1, wherein the pimavanserin is administered to post-treat between at least 60 minutes and 240 minutes after the administration or release of the 2C-B tartrate Form 1. In some embodiments, the serotonin receptor modulator is pimavanserin and the psychedelic is 2C-B tartrate Form 1, wherein the pimavanserin is administered to post-treat at least 90 minutes after the 2C-B tartrate Form 1. In some embodiments, the serotonin receptor modulator is pimavanserin and the psychedelic is 2C-B tartrate Form 1, wherein the pimavanserin is administered to post-treat at least 120 minutes after the 2C-B tartrate Form 1. In some embodiments, the serotonin receptor modulator is pimavanserin and the psychedelic is 2C-B tartrate Form 1, wherein the pimavanserin is administered to post-treat at least 150 minutes after the 2C-B tartrate Form 1. In some embodiments, the serotonin receptor modulator is pimavanserin and the psychedelic is 2C-B tartrate Form 1, wherein the pimavanserin is administered to post-treat between about 15 minutes and about 150 minutes after the 2C-B tartrate Form 1. In some embodiments, the serotonin receptor modulator is pimavanserin and the psychedelic is 2C-B tartrate Form 1, wherein the pimavanserin is administered to post-treat at least 180 minutes after the 2C-B tartrate Form 1. In some embodiments, the serotonin receptor modulator is pimavanserin and the psychedelic is 2C-B tartrate Form 1, wherein the pimavanserin is administered to post-treat at least 210 minutes after the 2C-B tartrate Form 1. In some embodiments, the serotonin receptor modulator is pimavanserin and the psychedelic is 2C-B tartrate Form 1, wherein the pimavanserin is administered to post-treat at least 240 minutes after the 2C-B tartrate Form 1. In some embodiments, the serotonin receptor modulator is pimavanserin and the psychedelic is 2C-B tartrate Form 1, wherein the pimavanserin is administered to post-treat at least 270 minutes after the 2C-B tartrate Form 1. In some embodiments, the serotonin receptor modulator is pimavanserin and the psychedelic is 2C-B tartrate Form 1, wherein the pimavanserin is administered to post-treat at least 300 minutes after the 2C-B tartrate Form 1. In some embodiments, the serotonin receptor modulator is pimavanserin and the psychedelic is 2C-B tartrate Form 1, wherein the pimavanserin is administered to post-treat at least 330 minutes after the 2C-B tartrate Form 1. In some embodiments, the serotonin receptor modulator is pimavanserin and the psychedelic is 2C-B tartrate Form 1, wherein the pimavanserin is administered to post-treat at least 360 minutes after the 2C-B tartrate Form 1. In some preferred embodiments, the serotonin receptor modulator is pimavanserin and the psychedelic is 2C-B tartrate Form 1, wherein pimavanserin is administered to post-treat between about 60 minutes and about 180 minutes after the administration of the 2C-B tartrate Form 1.

In some embodiments, the serotonin receptor modulator is nelotanserin and the psychedelic is 2C-B tartrate Form 1, wherein the nelotanserin is administered to post-treat at least 15 minutes after the administration of the 2C-B tartrate Form 1. In some embodiments, the serotonin receptor modulator is nelotanserin and the psychedelic is 2C-B tartrate Form 1, wherein the nelotanserin is administered to post-treat at least 30 minutes after the 2C-B tartrate Form 1. In some embodiments, the serotonin receptor modulator is nelotanserin and the psychedelic is 2C-B tartrate Form 1, wherein the nelotanserin is administered to post-treat between at least 60 minutes and 240 minutes after the administration or release of the 2C-B tartrate Form 1. In some embodiments, the serotonin receptor modulator is nelotanserin and the psychedelic is 2C-B tartrate Form 1, wherein the nelotanserin is administered to post-treat at least 90 minutes after the 2C-B tartrate Form 1. In some embodiments, the serotonin receptor modulator is nelotanserin and the psychedelic is 2C-B tartrate Form 1, wherein the nelotanserin is administered to post-treat at least 120 minutes after the 2C-B tartrate Form 1.

In some embodiments, the serotonin receptor modulator is nelotanserin and the psychedelic is 2C-B tartrate Form 1, wherein the nelotanserin is administered to post-treat at least 150 minutes after the 2C-B tartrate Form 1. In some embodiments, the serotonin receptor modulator is nelotanserin and the psychedelic is 2C-B tartrate Form 1, wherein the nelotanserin is administered to post-treat between about 15 minutes and about 150 minutes after the 2C-B tartrate Form 1. In some embodiments, the serotonin receptor modulator is nelotanserin and the psychedelic is 2C-B tartrate Form 1, wherein the nelotanserin is administered to post-treat at least 180 minutes after the 2C-B tartrate Form 1. In some embodiments, the serotonin receptor modulator is nelotanserin and the psychedelic is 2C-B tartrate Form 1, wherein the nelotanserin is administered to post-treat at least 210 minutes after the 2C-B tartrate Form 1. In some embodiments, the serotonin receptor modulator is nelotanserin and the psychedelic is 2C-B tartrate Form 1, wherein the nelotanserin is administered to post-treat at least 240 minutes after the 2C-B tartrate Form 1. In some embodiments, the serotonin receptor modulator is nelotanserin and the psychedelic is 2C-B tartrate Form 1, wherein the nelotanserin is administered to post-treat at least 270 minutes after the 2C-B tartrate Form 1.

In some embodiments, the serotonin receptor modulator is nelotanserin and the psychedelic is 2C-B tartrate Form 1, wherein the nelotanserin is administered to post-treat at least 300 minutes after the 2C-B tartrate Form 1. In some embodiments, the serotonin receptor modulator is nelotanserin and the psychedelic is 2C-B tartrate Form 1, wherein the nelotanserin is administered to post-treat at least 330 minutes after the 2C-B tartrate Form 1. In some embodiments, the serotonin receptor modulator is nelotanserin and the psychedelic is 2C-B tartrate Form 1, wherein the nelotanserin is administered to post-treat at least 360 minutes after the 2C-B tartrate Form 1. In some preferred embodiments, the serotonin receptor modulator is nelotanserin and the psychedelic is 2C-B tartrate Form 1, wherein nelotanserin is administered to post-treat between about 60 minutes and about 180 minutes after the administration of the 2C-B tartrate Form 1.

In some embodiments, the serotonin receptor modulator is pruvanserin and the psychedelic is 2C-B tartrate Form 1, wherein the pruvanserin is administered to post-treat at least 15 minutes after the administration of the 2C-B tartrate Form 1. In some embodiments, the serotonin receptor modulator is pruvanserin and the psychedelic is 2C-B tartrate Form 1, wherein the pruvanserin is administered to post-treat at least 30 minutes after the 2C-B tartrate Form 1. In some embodiments, the serotonin receptor modulator is pruvanserin and the psychedelic is 2C-B tartrate Form 1, wherein the pruvanserin is administered to post-treat between at least 60 minutes and 240 minutes after the administration or release of the 2C-B tartrate Form 1. In some embodiments, the serotonin receptor modulator is pruvanserin and the psychedelic is 2C-B tartrate Form 1, wherein the pruvanserin is administered to post-treat at least 90 minutes after the 2C-B tartrate Form 1. In some embodiments, the serotonin receptor modulator is pruvanserin and the psychedelic is 2C-B tartrate Form 1, wherein the pruvanserin is administered to post-treat at least 120 minutes after the 2C-B tartrate Form 1. In some embodiments, the serotonin receptor modulator is pruvanserin and the psychedelic is 2C-B tartrate Form 1, wherein the pruvanserin is administered to post-treat at least 150 minutes after the 2C-B tartrate Form 1. In some embodiments, the serotonin receptor modulator is pruvanserin and the psychedelic is 2C-B tartrate Form 1, wherein the pruvanserin is administered to post-treat between about 15 minutes and about 150 minutes after the 2C-B tartrate Form 1. In some embodiments, the serotonin receptor modulator is pruvanserin and the psychedelic is 2C-B tartrate Form 1, wherein the pruvanserin is administered to post-treat at least 180 minutes after the 2C-B tartrate Form 1. In some embodiments, the serotonin receptor modulator is pruvanserin and the psychedelic is 2C-B tartrate Form 1, wherein the pruvanserin is administered to post-treat at least 210 minutes after the 2C-B tartrate Form 1.

In some embodiments, the serotonin receptor modulator is pruvanserin and the psychedelic is 2C-B tartrate Form 1, wherein the pruvanserin is administered to post-treat at least 240 minutes after the 2C-B tartrate Form 1. In some embodiments, the serotonin receptor modulator is pruvanserin and the psychedelic is 2C-B tartrate Form 1, wherein the pruvanserin is administered to post-treat at least 270 minutes after the 2C-B tartrate Form 1. In some embodiments, the serotonin receptor modulator is pruvanserin and the psychedelic is 2C-B tartrate Form 1, wherein the pruvanserin is administered to post-treat at least 300 minutes after the 2C-B tartrate Form 1. In some embodiments, the serotonin receptor modulator is pruvanserin and the psychedelic is 2C-B tartrate Form 1, wherein the pruvanserin is administered to post-treat at least 330 minutes after the 2C-B tartrate Form 1. In some embodiments, the serotonin receptor modulator is pruvanserin and the psychedelic is 2C-B tartrate Form 1, wherein the pruvanserin is administered to post-treat at least 360 minutes after the 2C-B tartrate Form 1. In some preferred embodiments, the serotonin receptor modulator is pruvanserin and the psychedelic is 2C-B tartrate Form 1, wherein pruvanserin is administered to post-treat between about 60 minutes and about 180 minutes after the administration of the 2C-B tartrate Form 1.

In some embodiments, the serotonin receptor modulator is flibanserin, wherein the flibanserin is administered to post-treat at least 15 minutes post to the administration of the 2C-B tartrate Form 1. In some embodiments, the serotonin receptor modulator is flibanserin, wherein the flibanserin is administered to post-treat at least 30 minutes post to the 2C-B tartrate Form 1. In some embodiments, the serotonin receptor modulator is flibanserin, wherein the flibanserin is administered to post-treat between at least 60 minutes and 240 minutes post to the administration or release of the 2C-B tartrate Form 1. In some embodiments, the serotonin receptor modulator is flibanserin, wherein the flibanserin is administered to post-treat at least 90 minutes post to the 2C-B tartrate Form 1. In some embodiments, the serotonin receptor modulator is flibanserin, wherein the flibanserin is administered to post-treat at least 120 minutes post to the 2C-B tartrate Form 1. In some embodiments, the serotonin receptor modulator is flibanserin, wherein the flibanserin is administered to post-treat at least 150 minutes post to the 2C-B tartrate Form 1. In some embodiments, the serotonin receptor modulator is flibanserin, wherein the flibanserin is administered to post-treat between about 15 minutes and about 150 minutes post to the 2C-B tartrate Form 1. In some embodiments, the serotonin receptor modulator is flibanserin, wherein the flibanserin is administered to post-treat at least 180 minutes post to the 2C-B tartrate Form 1. In some embodiments, the serotonin receptor modulator is flibanserin, wherein the flibanserin is administered to post-treat at least 210 minutes post to the 2C-B tartrate Form 1.

In some embodiments, the serotonin receptor modulator is flibanserin, wherein the flibanserin is administered to post-treat at least 240 minutes post to the 2C-B tartrate Form 1. In some embodiments, the serotonin receptor modulator is flibanserin, wherein the flibanserin is administered to post-treat at least 270 minutes post to the 2C-B tartrate Form 1. In some embodiments, the serotonin receptor modulator is flibanserin, wherein the flibanserin is administered to post-treat at least 300 minutes post to the 2C-B tartrate Form 1. In some embodiments, the serotonin receptor modulator is flibanserin, wherein the flibanserin is administered to post-treat at least 330 minutes post to the 2C-B tartrate Form 1. In some embodiments, the serotonin receptor modulator is flibanserin, wherein the flibanserin is administered to post-treat at least 360 minutes post to the 2C-B tartrate Form 1. In some preferred embodiments, the serotonin receptor modulator is flibanserin, wherein flibanserin is administered to post-treat between about 60 minutes and about 180 minutes post to the administration of the 2C-B tartrate Form 1.

In some embodiments, the serotonin receptor modulator is olanzapine, wherein the olanzapine is administered to post-treat at least 15 minutes post to the administration of the 2C-B tartrate Form 1. In some embodiments, the serotonin receptor modulator is olanzapine, wherein the olanzapine is administered to post-treat at least 30 minutes post to the 2C-B tartrate Form 1. In some embodiments, the serotonin receptor modulator is olanzapine, wherein the olanzapine is administered to post-treat between at least 60 minutes and 240 minutes post to the administration or release of the 2C-B tartrate Form 1. In some embodiments, the serotonin receptor modulator is olanzapine, wherein the olanzapine is administered to post-treat at least 90 minutes post to the 2C-B tartrate Form 1. In some embodiments, the serotonin receptor modulator is olanzapine, wherein the olanzapine is administered to post-treat at least 120 minutes post to the 2C-B tartrate Form 1. In some embodiments, the serotonin receptor modulator is olanzapine, wherein the olanzapine is administered to post-treat at least 150 minutes post to the 2C-B tartrate Form 1. In some embodiments, the serotonin receptor modulator is olanzapine, wherein the olanzapine is administered to post-treat between about 15 minutes and about 150 minutes post to the 2C-B tartrate Form 1. In some embodiments, the serotonin receptor modulator is olanzapine, wherein the olanzapine is administered to post-treat at least 180 minutes post to the 2C-B tartrate Form 1. In some embodiments, the serotonin receptor modulator is olanzapine, wherein the olanzapine is administered to post-treat at least 210 minutes post to the 2C-B tartrate Form 1.

In some embodiments, the serotonin receptor modulator is olanzapine, wherein the olanzapine is administered to post-treat at least 240 minutes post to the 2C-B tartrate Form 1. In some embodiments, the serotonin receptor modulator is olanzapine, wherein the olanzapine is administered to post-treat at least 270 minutes post to the 2C-B tartrate Form 1. In some embodiments, the serotonin receptor modulator is olanzapine, wherein the olanzapine is administered to post-treat at least 300 minutes post to the 2C-B tartrate Form 1. In some embodiments, the serotonin receptor modulator is olanzapine, wherein the olanzapine is administered to post-treat at least 330 minutes post to the 2C-B tartrate Form 1. In some embodiments, the serotonin receptor modulator is olanzapine, wherein the olanzapine is administered to post-treat at least 360 minutes post to the 2C-B tartrate Form 1. In some preferred embodiments, the serotonin receptor modulator is olanzapine, wherein olanzapine is administered to post-treat between about 60 minutes and about 180 minutes post to the administration of the 2C-B tartrate Form 1.

In some embodiments, the serotonin receptor modulator is quetiapine, wherein the quetiapine is administered to post-treat at least 15 minutes post to the administration of the 2C-B tartrate Form 1. In some embodiments, the serotonin receptor modulator is quetiapine, wherein the quetiapine is administered to post-treat at least 30 minutes post to the 2C-B tartrate Form 1. In some embodiments, the serotonin receptor modulator is quetiapine, wherein the quetiapine is administered to post-treat between at least 60 minutes and 240 minutes post to the administration or release of the 2C-B tartrate Form 1. In some embodiments, the serotonin receptor modulator is quetiapine, wherein the quetiapine is administered to post-treat at least 90 minutes post to the 2C-B tartrate Form 1. In some embodiments, the serotonin receptor modulator is quetiapine, wherein the quetiapine is administered to post-treat at least 120 minutes post to the 2C-B tartrate Form 1. In some embodiments, the serotonin receptor modulator is quetiapine, wherein the quetiapine is administered to post-treat at least 150 minutes post to the 2C-B tartrate Form 1. In some embodiments, the serotonin receptor modulator is quetiapine, wherein the quetiapine is administered to post-treat between about 15 minutes and about 150 minutes post to the 2C-B tartrate Form 1. In some embodiments, the serotonin receptor modulator is quetiapine, wherein the quetiapine is administered to post-treat at least 180 minutes post to the 2C-B tartrate Form 1. In some embodiments, the serotonin receptor modulator is quetiapine, wherein the quetiapine is administered to post-treat at least 210 minutes post to the 2C-B tartrate Form 1.

In some embodiments, the serotonin receptor modulator is quetiapine, wherein the quetiapine is administered to post-treat at least 240 minutes post to the 2C-B tartrate Form 1. In some embodiments, the serotonin receptor modulator is quetiapine, wherein the quetiapine is administered to post-treat at least 270 minutes post to the 2C-B tartrate Form 1. In some embodiments, the serotonin receptor modulator is quetiapine, wherein the quetiapine is administered to post-treat at least 300 minutes post to the 2C-B tartrate Form 1. In some embodiments, the serotonin receptor modulator is quetiapine, wherein the quetiapine is administered to post-treat at least 330 minutes post to the 2C-B tartrate Form 1. In some embodiments, the serotonin receptor modulator is quetiapine, wherein the quetiapine is administered to post-treat at least 360 minutes post to the 2C-B tartrate Form 1. In some preferred embodiments, the serotonin receptor modulator is quetiapine, wherein quetiapine is administered to post-treat between about 60 minutes and about 180 minutes post to the administration of the 2C-B tartrate Form 1.

In some embodiments, the serotonin receptor modulator is risperidone, wherein the risperidone is administered to post-treat at least 15 minutes post to the administration of the 2C-B tartrate Form 1. In some embodiments, the serotonin receptor modulator is risperidone, wherein the risperidone is administered to post-treat at least 30 minutes post to the 2C-B tartrate Form 1. In some embodiments, the serotonin receptor modulator is risperidone, wherein the risperidone is administered to post-treat between at least 60 minutes and 240 minutes post to the administration or release of the 2C-B tartrate Form 1. In some embodiments, the serotonin receptor modulator is risperidone, wherein the risperidone is administered to post-treat at least 90 minutes post to the 2C-B tartrate Form 1. In some embodiments, the serotonin receptor modulator is risperidone, wherein the risperidone is administered to post-treat at least 120 minutes post to the 2C-B tartrate Form 1. In some embodiments, the serotonin receptor modulator is risperidone, wherein the risperidone is administered to post-treat at least 150 minutes post to the 2C-B tartrate Form 1. In some embodiments, the serotonin receptor modulator is risperidone, wherein the risperidone is administered to post-treat between about 15 minutes and about 150 minutes post to the 2C-B tartrate Form 1. In some embodiments, the serotonin receptor modulator is risperidone, wherein the risperidone is administered to post-treat at least 180 minutes post to the 2C-B tartrate Form 1. In some embodiments, the serotonin receptor modulator is risperidone, wherein the risperidone is administered to post-treat at least 210 minutes post to the 2C-B tartrate Form 1.

In some embodiments, the serotonin receptor modulator is risperidone, wherein the risperidone is administered to post-treat at least 240 minutes post to the 2C-B tartrate Form 1. In some embodiments, the serotonin receptor modulator is risperidone, wherein the risperidone is administered to post-treat at least 270 minutes post to the 2C-B tartrate Form 1. In some embodiments, the serotonin receptor modulator is risperidone, wherein the risperidone is administered to post-treat at least 300 minutes post to the 2C-B tartrate Form 1. In some embodiments, the serotonin receptor modulator is risperidone, wherein the risperidone is administered to post-treat at least 330 minutes post to the 2C-B tartrate Form 1. In some embodiments, the serotonin receptor modulator is risperidone, wherein the risperidone is administered to post-treat at least 360 minutes post to the 2C-B tartrate Form 1. In some preferred embodiments, the serotonin receptor modulator is risperidone, wherein risperidone is administered to post-treat between about 60 minutes and about 180 minutes post to the administration of the 2C-B tartrate Form 1.

In some embodiments, the serotonin receptor modulator is eplivanserin and the psychedelic is 2C-B HCl Form A, wherein the eplivanserin is administered to post-treat at least 15 minutes after the administration of the 2C-B HCl Form A. In some embodiments, the serotonin receptor modulator is eplivanserin and the psychedelic is 2C-B HCl Form A, wherein the eplivanserin is administered to post-treat between at least 30 minutes after and 360 minutes after the release or administration of the 2C-B HCl Form A. In some embodiments, the serotonin receptor modulator is eplivanserin and the psychedelic is 2C-B HCl Form A, wherein the eplivanserin is administered to post-treat between at least 60 minutes after and 360 minutes after the release or administration of the 2C-B HCl Form A. In some embodiments, the serotonin receptor modulator is eplivanserin and the psychedelic is 2C-B HCl Form A, wherein the eplivanserin is administered to post-treat between at least 90 minutes and 240 minutes after the 2C-B HCl Form A. In some embodiments, the serotonin receptor modulator is eplivanserin and the psychedelic is 2C-B HCl Form A, wherein the eplivanserin is administered to post-treat at least 120 minutes after the 2C-B HCl Form A.

In some embodiments, the serotonin receptor modulator is eplivanserin and the psychedelic is 2C-B HCl Form A, wherein the eplivanserin is administered to post-treat at least 150 minutes after the 2C-B HCl Form A. In some embodiments, the serotonin receptor modulator is eplivanserin and the psychedelic is 2C-B HCl Form A, wherein the eplivanserin is administered to post-treat between about 15 minutes and about 150 minutes after the 2C-B HCl Form A. In some embodiments, the serotonin receptor modulator is eplivanserin and the psychedelic is 2C-B HCl Form A, wherein the eplivanserin is administered to post-treat at least 180 minutes after the 2C-B HCl Form A. In some embodiments, the serotonin receptor modulator is eplivanserin and the psychedelic is 2C-B HCl Form A, wherein the eplivanserin is administered to post-treat at least 210 minutes after the 2C-B HCl Form A. In some embodiments, the serotonin receptor modulator is eplivanserin and the psychedelic is 2C-B HCl Form A, wherein the eplivanserin is administered to post-treat at least 240 minutes after the 2C-B HCl Form A.

In some embodiments, the serotonin receptor modulator is eplivanserin and the psychedelic is 2C-B HCl Form A, wherein the eplivanserin is administered to post-treat at least 270 minutes after the 2C-B HCl Form A. In some embodiments, the serotonin receptor modulator is eplivanserin and the psychedelic is 2C-B HCl Form A, wherein the eplivanserin is administered to post-treat at least 300 minutes after the 2C-B HCl Form A. In some embodiments, the serotonin receptor modulator is eplivanserin and the psychedelic is 2C-B HCl Form A, wherein the eplivanserin is administered to post-treat at least 330 minutes after the 2C-B HCl Form A. In some embodiments, the serotonin receptor modulator is eplivanserin and the psychedelic is 2C-B HCl Form A, wherein the eplivanserin is administered to post-treat at least 360 minutes after the 2C-B HCl Form A.

In some preferred embodiments, the serotonin receptor modulator is eplivanserin and the psychedelic is 2C-B HCl Form A, wherein eplivanserin is administered to post-treat between about 60 minutes and about 180 minutes after the administration of the 2C-B HCl Form A.

In some embodiments, the serotonin receptor modulator is volinanserin and the psychedelic is 2C-B HCl Form A, wherein the volinanserin is administered to post-treat a subject between at least 15 minutes and 360 minutes after the administration or release of the 2C-B HCl Form A. In some embodiments, the serotonin receptor modulator is volinanserin and the psychedelic is 2C-B HCl Form A, wherein the volinanserin is administered to post-treat between at least 30 minutes and 360 minutes after the administration or release of the 2C-B HCl Form A. In some embodiments, the serotonin receptor modulator is volinanserin and the psychedelic is 2C-B HCl Form A, wherein the volinanserin is administered to post-treat between at least 60 minutes and 240 minutes after the administration or release of the 2C-B HCl Form A. In some embodiments, the serotonin receptor modulator is volinanserin and the psychedelic is 2C-B HCl Form A, wherein the volinanserin is administered to post-treat at least 90 minutes after 2C-B HCl Form A. In some embodiments, the serotonin receptor modulator is volinanserin and the psychedelic is 2C-B HCl Form A, wherein the volinanserin is administered to post-treat at least 120 minutes after the 2C-B HCl Form A.

In some embodiments, the serotonin receptor modulator is volinanserin and the psychedelic is 2C-B HCl Form A, wherein the volinanserin is administered to post-treat at least 150 minutes after the 2C-B HCl Form A. In some embodiments, the serotonin receptor modulator is volinanserin and the psychedelic is 2C-B HCl Form A, wherein the volinanserin is administered to post-treat between about 15 minutes and about 150 minutes after the 2C-B HCl Form A. In some embodiments, the serotonin receptor modulator is volinanserin and the psychedelic is 2C-B HCl Form A, wherein the volinanserin is administered to post-treat at least 180 minutes after the 2C-B HCl Form A. In some embodiments, the serotonin receptor modulator is volinanserin and the psychedelic is 2C-B HCl Form A, wherein the volinanserin is administered to post-treat at least 210 minutes after the 2C-B HCl Form A. In some embodiments, the serotonin receptor modulator is volinanserin and the psychedelic is 2C-B HCl Form A, wherein the volinanserin is administered to post-treat at least 240 minutes after the 2C-B HCl Form A. In some embodiments, the serotonin receptor modulator is volinanserin and the psychedelic is 2C-B HCl Form A, wherein the volinanserin is administered to post-treat at least 270 minutes after the 2C-B HCl Form A. In some embodiments, the serotonin receptor modulator is volinanserin and the psychedelic is 2C-B HCl Form A, wherein the volinanserin is administered to post-treat at least 300 minutes after the 2C-B HCl Form A. In some embodiments, the serotonin receptor modulator is volinanserin and the psychedelic is 2C-B HCl Form A, wherein the volinanserin is administered to post-treat at least 330 minutes after the 2C-B HCl Form A. In some embodiments, the serotonin receptor modulator is volinanserin and the psychedelic is 2C-B HCl Form A, wherein the volinanserin is administered to post-treat at least 360 minutes after the 2C-B HCl Form A. In some preferred embodiments, the serotonin receptor modulator is volinanserin and the psychedelic is 2C-B HCl Form A, wherein volinanserin is administered to post-treat between about 60 minutes and about 180 minutes after the administration of the 2C-B HCl Form A.

In some embodiments, the serotonin receptor modulator is ketanserin and the psychedelic is 2C-B HCl Form A, wherein the ketanserin is administered to post-treat at least 15 minutes after the administration of the 2C-B HCl Form A. In some embodiments, the serotonin receptor modulator is ketanserin and the psychedelic is 2C-B HCl Form A, wherein the ketanserin is administered to post-treat between at least 30 minutes and 360 minutes after the administration or release of the 2C-B HCl Form A. In some embodiments, the serotonin receptor modulator is ketanserin and the psychedelic is 2C-B HCl Form A, wherein the ketanserin is administered to post-treat between at least 60 minutes and 240 minutes after the administration or release of the 2C-B HCl Form A. In some embodiments, the serotonin receptor modulator is ketanserin and the psychedelic is 2C-B HCl Form A, wherein the ketanserin is administered to post-treat at least 90 minutes after the 2C-B HCl Form A. In some embodiments, the serotonin receptor modulator is ketanserin and the psychedelic is 2C-B HCl Form A, wherein the ketanserin is administered to post-treat at least 120 minutes after the 2C-B HCl Form A. In some embodiments, the serotonin receptor modulator is ketanserin and the psychedelic is 2C-B HCl Form A, wherein the ketanserin is administered to post-treat at least 150 minutes after the 2C-B HCl Form A. In some embodiments, the serotonin receptor modulator is ketanserin and the psychedelic is 2C-B HCl Form A, wherein the ketanserin is administered to post-treat between about 15 minutes and about 150 minutes after the 2C-B HCl Form A. In some embodiments, the serotonin receptor modulator is ketanserin and the psychedelic is 2C-B HCl Form A, wherein the ketanserin is administered to post-treat at least 180 minutes after the 2C-B HCl Form A. In some embodiments, the serotonin receptor modulator is ketanserin and the psychedelic is 2C-B HCl Form A, wherein the ketanserin is administered to post-treat at least 210 minutes after the 2C-B HCl Form A. In some embodiments, the serotonin receptor modulator is ketanserin and the psychedelic is 2C-B HCl Form A, wherein the ketanserin is administered to post-treat at least 240 minutes after the 2C-B HCl Form A. In some embodiments, the serotonin receptor modulator is ketanserin and the psychedelic is 2C-B HCl Form A, wherein the ketanserin is administered to post-treat at least 270 minutes after the 2C-B HCl Form A. In some embodiments, the serotonin receptor modulator is ketanserin and the psychedelic is 2C-B HCl Form A, wherein the ketanserin is administered to post-treat at least 300 minutes after the 2C-B HCl Form A. In some embodiments, the serotonin receptor modulator is ketanserin and the psychedelic is 2C-B HCl Form A, wherein the ketanserin is administered to post-treat at least 330 minutes after the 2C-B HCl Form A. In some embodiments, the serotonin receptor modulator is ketanserin and the psychedelic is 2C-B HCl Form A, wherein the ketanserin is administered to post-treat at least 360 minutes after the 2C-B HCl Form A. In some preferred embodiments, the serotonin receptor modulator is ketanserin and the psychedelic is 2C-B HCl Form A, wherein ketanserin is administered to post-treat between about 60 minutes and about 180 minutes after the administration of the 2C-B HCl Form A.

In some embodiments, the serotonin receptor modulator is ritanserin and the psychedelic is 2C-B HCl Form A, wherein the ritanserin is administered to post-treat at least 15 minutes after the administration of the 2C-B HCl Form A. In some embodiments, the serotonin receptor modulator is ritanserin and the psychedelic is 2C-B HCl Form A, wherein the ritanserin is administered to post-treat at least 30 minutes after the 2C-B HCl Form A. In some embodiments, the serotonin receptor modulator is ritanserin and the psychedelic is 2C-B HCl Form A, wherein the ritanserin is administered to post-treat between at least 60 minutes and 240 minutes after the administration or release of the 2C-B HCl Form A. In some embodiments, the serotonin receptor modulator is ritanserin and the psychedelic is 2C-B HCl Form A, wherein the ritanserin is administered to post-treat at least 90 minutes after the 2C-B HCl Form A. In some embodiments, the serotonin receptor modulator is ritanserin and the psychedelic is 2C-B HCl Form A, wherein the ritanserin is administered to post-treat at least 120 minutes after the 2C-B HCl Form A. In some embodiments, the serotonin receptor modulator is ritanserin and the psychedelic is 2C-B HCl Form A, wherein the ritanserin is administered to post-treat at least 150 minutes after the 2C-B HCl Form A. In some embodiments, the serotonin receptor modulator is ritanserin and the psychedelic is 2C-B HCl Form A, wherein the ritanserin is administered to post-treat between about 15 minutes and about 150 minutes after the 2C-B HCl Form A. In some embodiments, the serotonin receptor modulator is ritanserin and the psychedelic is 2C-B HCl Form A, wherein the ritanserin is administered to post-treat at least 180 minutes after the 2C-B HCl Form A. In some embodiments, the serotonin receptor modulator is ritanserin and the psychedelic is 2C-B HCl Form A, wherein the ritanserin is administered to post-treat at least 210 minutes after the 2C-B HCl Form A. In some embodiments, the serotonin receptor modulator is ritanserin and the psychedelic is 2C-B HCl Form A, wherein the ritanserin is administered to post-treat at least 240 minutes after the 2C-B HCl Form A. In some embodiments, the serotonin receptor modulator is ritanserin and the psychedelic is 2C-B HCl Form A, wherein the ritanserin is administered to post-treat at least 270 minutes after the 2C-B HCl Form A. In some embodiments, the serotonin receptor modulator is ritanserin and the psychedelic is 2C-B HCl Form A, wherein the ritanserin is administered to post-treat at least 300 minutes after the 2C-B HCl Form A. In some embodiments, the serotonin receptor modulator is ritanserin and the psychedelic is 2C-B HCl Form A, wherein the ritanserin is administered to post-treat at least 330 minutes after the 2C-B HCl Form A. In some embodiments, the serotonin receptor modulator is ritanserin and the psychedelic is 2C-B HCl Form A, wherein the ritanserin is administered to post-treat at least 360 minutes after the 2C-B HCl Form A. In some preferred embodiments, the serotonin receptor modulator is ritanserin and the psychedelic is 2C-B HCl Form A, wherein ritanserin is administered to post-treat between about 60 minutes and about 180 minutes after the administration of the 2C-B HCl Form A.

In some embodiments, the serotonin receptor modulator is pimavanserin and the psychedelic is 2C-B HCl Form A, wherein the pimavanserin is administered to post-treat at least 15 minutes after the administration of the 2C-B HCl Form A. In some embodiments, the serotonin receptor modulator is pimavanserin and the psychedelic is 2C-B HCl Form A, wherein the pimavanserin is administered to post-treat at least 30 minutes after the 2C-B HCl Form A. In some embodiments, the serotonin receptor modulator is pimavanserin and the psychedelic is 2C-B HCl Form A, wherein the pimavanserin is administered to post-treat between at least 60 minutes and 240 minutes after the administration or release of the 2C-B HCl Form A. In some embodiments, the serotonin receptor modulator is pimavanserin and the psychedelic is 2C-B HCl Form A, wherein the pimavanserin is administered to post-treat at least 90 minutes after the 2C-B HCl Form A. In some embodiments, the serotonin receptor modulator is pimavanserin and the psychedelic is 2C-B HCl Form A, wherein the pimavanserin is administered to post-treat at least 120 minutes after the 2C-B HCl Form A. In some embodiments, the serotonin receptor modulator is pimavanserin and the psychedelic is 2C-B HCl Form A, wherein the pimavanserin is administered to post-treat at least 150 minutes after the 2C-B HCl Form A. In some embodiments, the serotonin receptor modulator is pimavanserin and the psychedelic is 2C-B HCl Form A, wherein the pimavanserin is administered to post-treat between about 15 minutes and about 150 minutes after the 2C-B HCl Form A. In some embodiments, the serotonin receptor modulator is pimavanserin and the psychedelic is 2C-B HCl Form A, wherein the pimavanserin is administered to post-treat at least 180 minutes after the 2C-B HCl Form A. In some embodiments, the serotonin receptor modulator is pimavanserin and the psychedelic is 2C-B HCl Form A, wherein the pimavanserin is administered to post-treat at least 210 minutes after the 2C-B HCl Form A. In some embodiments, the serotonin receptor modulator is pimavanserin and the psychedelic is 2C-B HCl Form A, wherein the pimavanserin is administered to post-treat at least 240 minutes after the 2C-B HCl Form A. In some embodiments, the serotonin receptor modulator is pimavanserin and the psychedelic is 2C-B HCl Form A, wherein the pimavanserin is administered to post-treat at least 270 minutes after the 2C-B HCl Form A. In some embodiments, the serotonin receptor modulator is pimavanserin and the psychedelic is 2C-B HCl Form A, wherein the pimavanserin is administered to post-treat at least 300 minutes after the 2C-B HCl Form A. In some embodiments, the serotonin receptor modulator is pimavanserin and the psychedelic is 2C-B HCl Form A, wherein the pimavanserin is administered to post-treat at least 330 minutes after the 2C-B HCl Form A. In some embodiments, the serotonin receptor modulator is pimavanserin and the psychedelic is 2C-B HCl Form A, wherein the pimavanserin is administered to post-treat at least 360 minutes after the 2C-B HCl Form A. In some preferred embodiments, the serotonin receptor modulator is pimavanserin and the psychedelic is 2C-B HCl Form A, wherein pimavanserin is administered to post-treat between about 60 minutes and about 180 minutes after the administration of the 2C-B HCl Form A.

In some embodiments, the serotonin receptor modulator is nelotanserin and the psychedelic is 2C-B HCl Form A, wherein the nelotanserin is administered to post-treat at least 15 minutes after the administration of the 2C-B HCl Form A. In some embodiments, the serotonin receptor modulator is nelotanserin and the psychedelic is 2C-B HCl Form A, wherein the nelotanserin is administered to post-treat at least 30 minutes after the 2C-B HCl Form A. In some embodiments, the serotonin receptor modulator is nelotanserin and the psychedelic is 2C-B HCl Form A, wherein the nelotanserin is administered to post-treat between at least 60 minutes and 240 minutes after the administration or release of the 2C-B HCl Form A. In some embodiments, the serotonin receptor modulator is nelotanserin and the psychedelic is 2C-B HCl Form A, wherein the nelotanserin is administered to post-treat at least 90 minutes after the 2C-B HCl Form A. In some embodiments, the serotonin receptor modulator is nelotanserin and the psychedelic is 2C-B HCl Form A, wherein the nelotanserin is administered to post-treat at least 120 minutes after the 2C-B HCl Form A.

In some embodiments, the serotonin receptor modulator is nelotanserin and the psychedelic is 2C-B HCl Form A, wherein the nelotanserin is administered to post-treat at least 150 minutes after the 2C-B HCl Form A. In some embodiments, the serotonin receptor modulator is nelotanserin and the psychedelic is 2C-B HCl Form A, wherein the nelotanserin is administered to post-treat between about 15 minutes and about 150 minutes after the 2C-B HCl Form A. In some embodiments, the serotonin receptor modulator is nelotanserin and the psychedelic is 2C-B HCl Form A, wherein the nelotanserin is administered to post-treat at least 180 minutes after the 2C-B HCl Form A. In some embodiments, the serotonin receptor modulator is nelotanserin and the psychedelic is 2C-B HCl Form A, wherein the nelotanserin is administered to post-treat at least 210 minutes after the 2C-B HCl Form A. In some embodiments, the serotonin receptor modulator is nelotanserin and the psychedelic is 2C-B HCl Form A, wherein the nelotanserin is administered to post-treat at least 240 minutes after the 2C-B HCl Form A. In some embodiments, the serotonin receptor modulator is nelotanserin and the psychedelic is 2C-B HCl Form A, wherein the nelotanserin is administered to post-treat at least 270 minutes after the 2C-B HCl Form A.

In some embodiments, the serotonin receptor modulator is nelotanserin and the psychedelic is 2C-B HCl Form A, wherein the nelotanserin is administered to post-treat at least 300 minutes after the 2C-B HCl Form A. In some embodiments, the serotonin receptor modulator is nelotanserin and the psychedelic is 2C-B HCl Form A, wherein the nelotanserin is administered to post-treat at least 330 minutes after the 2C-B HCl Form A. In some embodiments, the serotonin receptor modulator is nelotanserin and the psychedelic is 2C-B HCl Form A, wherein the nelotanserin is administered to post-treat at least 360 minutes after the 2C-B HCl Form A. In some preferred embodiments, the serotonin receptor modulator is nelotanserin and the psychedelic is 2C-B HCl Form A, wherein nelotanserin is administered to post-treat between about 60 minutes and about 180 minutes after the administration of the 2C-B HCl Form A.

In some embodiments, the serotonin receptor modulator is pruvanserin and the psychedelic is 2C-B HCl Form A, wherein the pruvanserin is administered to post-treat at least 15 minutes after the administration of the 2C-B HCl Form A. In some embodiments, the serotonin receptor modulator is pruvanserin and the psychedelic is 2C-B HCl Form A, wherein the pruvanserin is administered to post-treat at least 30 minutes after the 2C-B HCl Form A. In some embodiments, the serotonin receptor modulator is pruvanserin and the psychedelic is 2C-B HCl Form A, wherein the pruvanserin is administered to post-treat between at least 60 minutes and 240 minutes after the administration or release of the 2C-B HCl Form A. In some embodiments, the serotonin receptor modulator is pruvanserin and the psychedelic is 2C-B HCl Form A, wherein the pruvanserin is administered to post-treat at least 90 minutes after the 2C-B HCl Form A. In some embodiments, the serotonin receptor modulator is pruvanserin and the psychedelic is 2C-B HCl Form A, wherein the pruvanserin is administered to post-treat at least 120 minutes after the 2C-B HCl Form A. In some embodiments, the serotonin receptor modulator is pruvanserin and the psychedelic is 2C-B HCl Form A, wherein the pruvanserin is administered to post-treat at least 150 minutes after the 2C-B HCl Form A. In some embodiments, the serotonin receptor modulator is pruvanserin and the psychedelic is 2C-B HCl Form A, wherein the pruvanserin is administered to post-treat between about 15 minutes and about 150 minutes after the 2C-B HCl Form A. In some embodiments, the serotonin receptor modulator is pruvanserin and the psychedelic is 2C-B HCl Form A, wherein the pruvanserin is administered to post-treat at least 180 minutes after the 2C-B HCl Form A. In some embodiments, the serotonin receptor modulator is pruvanserin and the psychedelic is 2C-B HCl Form A, wherein the pruvanserin is administered to post-treat at least 210 minutes after the 2C-B HCl Form A.

In some embodiments, the serotonin receptor modulator is pruvanserin and the psychedelic is 2C-B HCl Form A, wherein the pruvanserin is administered to post-treat at least 240 minutes after the 2C-B HCl Form A. In some embodiments, the serotonin receptor modulator is pruvanserin and the psychedelic is 2C-B HCl Form A, wherein the pruvanserin is administered to post-treat at least 270 minutes after the 2C-B HCl Form A. In some embodiments, the serotonin receptor modulator is pruvanserin and the psychedelic is 2C-B HCl Form A, wherein the pruvanserin is administered to post-treat at least 300 minutes after the 2C-B HCl Form A. In some embodiments, the serotonin receptor modulator is pruvanserin and the psychedelic is 2C-B HCl Form A, wherein the pruvanserin is administered to post-treat at least 330 minutes after the 2C-B HCl Form A. In some embodiments, the serotonin receptor modulator is pruvanserin and the psychedelic is 2C-B HCl Form A, wherein the pruvanserin is administered to post-treat at least 360 minutes after the 2C-B HCl Form A. In some preferred embodiments, the serotonin receptor modulator is pruvanserin and the psychedelic is 2C-B HCl Form A, wherein pruvanserin is administered to post-treat between about 60 minutes and about 180 minutes after the administration of the 2C-B HCl Form A.

In some embodiments, the serotonin receptor modulator is flibanserin, wherein the flibanserin is administered to post-treat at least 15 minutes post to the administration of the 2C-B HCl Form A. In some embodiments, the serotonin receptor modulator is flibanserin, wherein the flibanserin is administered to post-treat at least 30 minutes post to the 2C-B HCl Form A. In some embodiments, the serotonin receptor modulator is flibanserin, wherein the flibanserin is administered to post-treat between at least 60 minutes and 240 minutes post to the administration or release of the 2C-B HCl Form A. In some embodiments, the serotonin receptor modulator is flibanserin, wherein the flibanserin is administered to post-treat at least 90 minutes post to the 2C-B HCl Form A. In some embodiments, the serotonin receptor modulator is flibanserin, wherein the flibanserin is administered to post-treat at least 120 minutes post to the 2C-B HCl Form A. In some embodiments, the serotonin receptor modulator is flibanserin, wherein the flibanserin is administered to post-treat at least 150 minutes post to the 2C-B HCl Form A. In some embodiments, the serotonin receptor modulator is flibanserin, wherein the flibanserin is administered to post-treat between about 15 minutes and about 150 minutes post to the 2C-B HCl Form A. In some embodiments, the serotonin receptor modulator is flibanserin, wherein the flibanserin is administered to post-treat at least 180 minutes post to the 2C-B HCl Form A. In some embodiments, the serotonin receptor modulator is flibanserin, wherein the flibanserin is administered to post-treat at least 210 minutes post to the 2C-B HCl Form A.

In some embodiments, the serotonin receptor modulator is flibanserin, wherein the flibanserin is administered to post-treat at least 240 minutes post to the 2C-B HCl Form A. In some embodiments, the serotonin receptor modulator is flibanserin, wherein the flibanserin is administered to post-treat at least 270 minutes post to the 2C-B HCl Form A. In some embodiments, the serotonin receptor modulator is flibanserin, wherein the flibanserin is administered to post-treat at least 300 minutes post to the 2C-B HCl Form A. In some embodiments, the serotonin receptor modulator is flibanserin, wherein the flibanserin is administered to post-treat at least 330 minutes post to the 2C-B HCl Form A. In some embodiments, the serotonin receptor modulator is flibanserin, wherein the flibanserin is administered to post-treat at least 360 minutes post to the 2C-B HCl Form A. In some preferred embodiments, the serotonin receptor modulator is flibanserin, wherein flibanserin is administered to post-treat between about 60 minutes and about 180 minutes post to the administration of the 2C-B HCl Form A.

In some embodiments, the serotonin receptor modulator is olanzapine, wherein the olanzapine is administered to post-treat at least 15 minutes post to the administration of the 2C-B HCl Form A. In some embodiments, the serotonin receptor modulator is olanzapine, wherein the olanzapine is administered to post-treat at least 30 minutes post to the 2C-B HCl Form A. In some embodiments, the serotonin receptor modulator is olanzapine, wherein the olanzapine is administered to post-treat between at least 60 minutes and 240 minutes post to the administration or release of the 2C-B HCl Form A. In some embodiments, the serotonin receptor modulator is olanzapine, wherein the olanzapine is administered to post-treat at least 90 minutes post to the 2C-B HCl Form A. In some embodiments, the serotonin receptor modulator is olanzapine, wherein the olanzapine is administered to post-treat at least 120 minutes post to the 2C-B HCl Form A. In some embodiments, the serotonin receptor modulator is olanzapine, wherein the olanzapine is administered to post-treat at least 150 minutes post to the 2C-B HCl Form A. In some embodiments, the serotonin receptor modulator is olanzapine, wherein the olanzapine is administered to post-treat between about 15 minutes and about 150 minutes post to the 2C-B HCl Form A. In some embodiments, the serotonin receptor modulator is olanzapine, wherein the olanzapine is administered to post-treat at least 180 minutes post to the 2C-B HCl Form A. In some embodiments, the serotonin receptor modulator is olanzapine, wherein the olanzapine is administered to post-treat at least 210 minutes post to the 2C-B HCl Form A.

In some embodiments, the serotonin receptor modulator is olanzapine, wherein the olanzapine is administered to post-treat at least 240 minutes post to the 2C-B HCl Form A. In some embodiments, the serotonin receptor modulator is olanzapine, wherein the olanzapine is administered to post-treat at least 270 minutes post to the 2C-B HCl Form A. In some embodiments, the serotonin receptor modulator is olanzapine, wherein the olanzapine is administered to post-treat at least 300 minutes post to the 2C-B HCl Form A. In some embodiments, the serotonin receptor modulator is olanzapine, wherein the olanzapine is administered to post-treat at least 330 minutes post to the 2C-B HCl Form A. In some embodiments, the serotonin receptor modulator is olanzapine, wherein the olanzapine is administered to post-treat at least 360 minutes post to the 2C-B HCl Form A. In some preferred embodiments, the serotonin receptor modulator is olanzapine, wherein olanzapine is administered to post-treat between about 60 minutes and about 180 minutes post to the administration of the 2C-B HCl Form A.

In some embodiments, the serotonin receptor modulator is quetiapine, wherein the quetiapine is administered to post-treat at least 15 minutes post to the administration of the 2C-B HCl Form A. In some embodiments, the serotonin receptor modulator is quetiapine, wherein the quetiapine is administered to post-treat at least 30 minutes post to the 2C-B HCl Form A. In some embodiments, the serotonin receptor modulator is quetiapine, wherein the quetiapine is administered to post-treat between at least 60 minutes and 240 minutes post to the administration or release of the 2C-B HCl Form A. In some embodiments, the serotonin receptor modulator is quetiapine, wherein the quetiapine is administered to post-treat at least 90 minutes post to the 2C-B HCl Form A. In some embodiments, the serotonin receptor modulator is quetiapine, wherein the quetiapine is administered to post-treat at least 120 minutes post to the 2C-B HCl Form A. In some embodiments, the serotonin receptor modulator is quetiapine, wherein the quetiapine is administered to post-treat at least 150 minutes post to the 2C-B HCl Form A. In some embodiments, the serotonin receptor modulator is quetiapine, wherein the quetiapine is administered to post-treat between about 15 minutes and about 150 minutes post to the 2C-B HCl Form A. In some embodiments, the serotonin receptor modulator is quetiapine, wherein the quetiapine is administered to post-treat at least 180 minutes post to the 2C-B HCl Form A. In some embodiments, the serotonin receptor modulator is quetiapine, wherein the quetiapine is administered to post-treat at least 210 minutes post to the 2C-B HCl Form A.

In some embodiments, the serotonin receptor modulator is quetiapine, wherein the quetiapine is administered to post-treat at least 240 minutes post to the 2C-B HCl Form A. In some embodiments, the serotonin receptor modulator is quetiapine, wherein the quetiapine is administered to post-treat at least 270 minutes post to the 2C-B HCl Form A. In some embodiments, the serotonin receptor modulator is quetiapine, wherein the quetiapine is administered to post-treat at least 300 minutes post to the 2C-B HCl Form A. In some embodiments, the serotonin receptor modulator is quetiapine, wherein the quetiapine is administered to post-treat at least 330 minutes post to the 2C-B HCl Form A. In some embodiments, the serotonin receptor modulator is quetiapine, wherein the quetiapine is administered to post-treat at least 360 minutes post to the 2C-B HCl Form A. In some preferred embodiments, the serotonin receptor modulator is quetiapine, wherein quetiapine is administered to post-treat between about 60 minutes and about 180 minutes post to the administration of the 2C-B HCl Form A.

In some embodiments, the serotonin receptor modulator is risperidone, wherein the risperidone is administered to post-treat at least 15 minutes post to the administration of the 2C-B HCl Form A. In some embodiments, the serotonin receptor modulator is risperidone, wherein the risperidone is administered to post-treat at least 30 minutes post to the 2C-B HCl Form A. In some embodiments, the serotonin receptor modulator is risperidone, wherein the risperidone is administered to post-treat between at least 60 minutes and 240 minutes post to the administration or release of the 2C-B HCl Form A. In some embodiments, the serotonin receptor modulator is risperidone, wherein the risperidone is administered to post-treat at least 90 minutes post to the 2C-B HCl Form A. In some embodiments, the serotonin receptor modulator is risperidone, wherein the risperidone is administered to post-treat at least 120 minutes post to the 2C-B HCl Form A. In some embodiments, the serotonin receptor modulator is risperidone, wherein the risperidone is administered to post-treat at least 150 minutes post to the 2C-B HCl Form A. In some embodiments, the serotonin receptor modulator is risperidone, wherein the risperidone is administered to post-treat between about 15 minutes and about 150 minutes post to the 2C-B HCl Form A. In some embodiments, the serotonin receptor modulator is risperidone, wherein the risperidone is administered to post-treat at least 180 minutes post to the 2C-B HCl Form A. In some embodiments, the serotonin receptor modulator is risperidone, wherein the risperidone is administered to post-treat at least 210 minutes post to the 2C-B HCl Form A.

In some embodiments, the serotonin receptor modulator is risperidone, wherein the risperidone is administered to post-treat at least 240 minutes post to the 2C-B HCl Form A. In some embodiments, the serotonin receptor modulator is risperidone, wherein the risperidone is administered to post-treat at least 270 minutes post to the 2C-B HCl Form A. In some embodiments, the serotonin receptor modulator is risperidone, wherein the risperidone is administered to post-treat at least 300 minutes post to the 2C-B HCl Form A. In some embodiments, the serotonin receptor modulator is risperidone, wherein the risperidone is administered to post-treat at least 330 minutes post to the 2C-B HCl Form A. In some embodiments, the serotonin receptor modulator is risperidone, wherein the risperidone is administered to post-treat at least 360 minutes post to the 2C-B HCl Form A. In some preferred embodiments, the serotonin receptor modulator is risperidone, wherein risperidone is administered to post-treat between about 60 minutes and about 180 minutes post to the administration of the 2C-B HCl Form A.

In some embodiments, the serotonin receptor modulator is eplivanserin and the psychedelic is 2C-B HCl Form C, wherein the eplivanserin is administered to post-treat at least 15 minutes after the administration of the 2C-B HCl Form C. In some embodiments, the serotonin receptor modulator is eplivanserin and the psychedelic is 2C-B HCl Form C, wherein the eplivanserin is administered to post-treat between at least 30 minutes after and 360 minutes after the release or administration of the 2C-B HCl Form C. In some embodiments, the serotonin receptor modulator is eplivanserin and the psychedelic is 2C-B HCl Form C, wherein the eplivanserin is administered to post-treat between at least 60 minutes after and 360 minutes after the release or administration of the 2C-B HCl Form C. In some embodiments, the serotonin receptor modulator is eplivanserin and the psychedelic is 2C-B HCl Form C, wherein the eplivanserin is administered to post-treat between at least 90 minutes and 240 minutes after the 2C-B HCl Form C. In some embodiments, the serotonin receptor modulator is eplivanserin and the psychedelic is 2C-B HCl Form C, wherein the eplivanserin is administered to post-treat at least 120 minutes after the 2C-B HCl Form C.

In some embodiments, the serotonin receptor modulator is eplivanserin and the psychedelic is 2C-B HCl Form C, wherein the eplivanserin is administered to post-treat at least 150 minutes after the 2C-B HCl Form C. In some embodiments, the serotonin receptor modulator is eplivanserin and the psychedelic is 2C-B HCl Form C, wherein the eplivanserin is administered to post-treat between about 15 minutes and about 150 minutes after the 2C-B HCl Form C. In some embodiments, the serotonin receptor modulator is eplivanserin and the psychedelic is 2C-B HCl Form C, wherein the eplivanserin is administered to post-treat at least 180 minutes after the 2C-B HCl Form C. In some embodiments, the serotonin receptor modulator is eplivanserin and the psychedelic is 2C-B HCl Form C, wherein the eplivanserin is administered to post-treat at least 210 minutes after the 2C-B HCl Form C. In some embodiments, the serotonin receptor modulator is eplivanserin and the psychedelic is 2C-B HCl Form C, wherein the eplivanserin is administered to post-treat at least 240 minutes after the 2C-B HCl Form C.

In some embodiments, the serotonin receptor modulator is eplivanserin and the psychedelic is 2C-B HCl Form C, wherein the eplivanserin is administered to post-treat at least 270 minutes after the 2C-B HCl Form C. In some embodiments, the serotonin receptor modulator is eplivanserin and the psychedelic is 2C-B HCl Form C, wherein the eplivanserin is administered to post-treat at least 300 minutes after the 2C-B HCl Form C. In some embodiments, the serotonin receptor modulator is eplivanserin and the psychedelic is 2C-B HCl Form C, wherein the eplivanserin is administered to post-treat at least 330 minutes after the 2C-B HCl Form C. In some embodiments, the serotonin receptor modulator is eplivanserin and the psychedelic is 2C-B HCl Form C, wherein the eplivanserin is administered to post-treat at least 360 minutes after the 2C-B HCl Form C.

In some preferred embodiments, the serotonin receptor modulator is eplivanserin and the psychedelic is 2C-B HCl Form C, wherein eplivanserin is administered to post-treat between about 60 minutes and about 180 minutes after the administration of the 2C-B HCl Form C. In some embodiments, the serotonin receptor modulator is volinanserin and the psychedelic is 2C-B HCl Form C, wherein the volinanserin is administered to post-treat a subject between at least 15 minutes and 360 minutes after the administration or release of the 2C-B HCl Form C. In some embodiments, the serotonin receptor modulator is volinanserin and the psychedelic is 2C-B HCl Form C, wherein the volinanserin is administered to post-treat between at least 30 minutes and 360 minutes after the administration or release of the 2C-B HCl Form C. In some embodiments, the serotonin receptor modulator is volinanserin and the psychedelic is 2C-B HCl Form C, wherein the volinanserin is administered to post-treat between at least 60 minutes and 240 minutes after the administration or release of the 2C-B HCl Form C. In some embodiments, the serotonin receptor modulator is volinanserin and the psychedelic is 2C-B HCl Form C, wherein the volinanserin is administered to post-treat at least 90 minutes after 2C-B HCl Form C. In some embodiments, the serotonin receptor modulator is volinanserin and the psychedelic is 2C-B HCl Form C, wherein the volinanserin is administered to post-treat at least 120 minutes after the 2C-B HCl Form C.

In some embodiments, the serotonin receptor modulator is volinanserin and the psychedelic is 2C-B HCl Form C, wherein the volinanserin is administered to post-treat at least 150 minutes after the 2C-B HCl Form C. In some embodiments, the serotonin receptor modulator is volinanserin and the psychedelic is 2C-B HCl Form C, wherein the volinanserin is administered to post-treat between about 15 minutes and about 150 minutes after the 2C-B HCl Form C. In some embodiments, the serotonin receptor modulator is volinanserin and the psychedelic is 2C-B HCl Form C, wherein the volinanserin is administered to post-treat at least 180 minutes after the 2C-B HCl Form C. In some embodiments, the serotonin receptor modulator is volinanserin and the psychedelic is 2C-B HCl Form C, wherein the volinanserin is administered to post-treat at least 210 minutes after the 2C-B HCl Form C. In some embodiments, the serotonin receptor modulator is volinanserin and the psychedelic is 2C-B HCl Form C, wherein the volinanserin is administered to post-treat at least 240 minutes after the 2C-B HCl Form C. In some embodiments, the serotonin receptor modulator is volinanserin and the psychedelic is 2C-B HCl Form C, wherein the volinanserin is administered to post-treat at least 270 minutes after the 2C-B HCl Form C. In some embodiments, the serotonin receptor modulator is volinanserin and the psychedelic is 2C-B HCl Form C, wherein the volinanserin is administered to post-treat at least 300 minutes after the 2C-B HCl Form C. In some embodiments, the serotonin receptor modulator is volinanserin and the psychedelic is 2C-B HCl Form C, wherein the volinanserin is administered to post-treat at least 330 minutes after the 2C-B HCl Form C. In some embodiments, the serotonin receptor modulator is volinanserin and the psychedelic is 2C-B HCl Form C, wherein the volinanserin is administered to post-treat at least 360 minutes after the 2C-B HCl Form C. In some preferred embodiments, the serotonin receptor modulator is volinanserin and the psychedelic is 2C-B HCl Form C, wherein volinanserin is administered to post-treat between about 60 minutes and about 180 minutes after the administration of the 2C-B HCl Form C.

In some embodiments, the serotonin receptor modulator is ketanserin and the psychedelic is 2C-B HCl Form C, wherein the ketanserin is administered to post-treat at least 15 minutes after the administration of the 2C-B HCl Form C. In some embodiments, the serotonin receptor modulator is ketanserin and the psychedelic is 2C-B HCl Form C, wherein the ketanserin is administered to post-treat between at least 30 minutes and 360 minutes after the administration or release of the 2C-B HCl Form C. In some embodiments, the serotonin receptor modulator is ketanserin and the psychedelic is 2C-B HCl Form C, wherein the ketanserin is administered to post-treat between at least 60 minutes and 240 minutes after the administration or release of the 2C-B HCl Form C. In some embodiments, the serotonin receptor modulator is ketanserin and the psychedelic is 2C-B HCl Form C, wherein the ketanserin is administered to post-treat at least 90 minutes after the 2C-B HCl Form C. In some embodiments, the serotonin receptor modulator is ketanserin and the psychedelic is 2C-B HCl Form C, wherein the ketanserin is administered to post-treat at least 120 minutes after the 2C-B HCl Form C. In some embodiments, the serotonin receptor modulator is ketanserin and the psychedelic is 2C-B HCl Form C, wherein the ketanserin is administered to post-treat at least 150 minutes after the 2C-B HCl Form C. In some embodiments, the serotonin receptor modulator is ketanserin and the psychedelic is 2C-B HCl Form C, wherein the ketanserin is administered to post-treat between about 15 minutes and about 150 minutes after the 2C-B HCl Form C. In some embodiments, the serotonin receptor modulator is ketanserin and the psychedelic is 2C-B HCl Form C, wherein the ketanserin is administered to post-treat at least 180 minutes after the 2C-B HCl Form C. In some embodiments, the serotonin receptor modulator is ketanserin and the psychedelic is 2C-B HCl Form C, wherein the ketanserin is administered to post-treat at least 210 minutes after the 2C-B HCl Form C. In some embodiments, the serotonin receptor modulator is ketanserin and the psychedelic is 2C-B HCl Form C, wherein the ketanserin is administered to post-treat at least 240 minutes after the 2C-B HCl Form C. In some embodiments, the serotonin receptor modulator is ketanserin and the psychedelic is 2C-B HCl Form C, wherein the ketanserin is administered to post-treat at least 270 minutes after the 2C-B HCl Form C. In some embodiments, the serotonin receptor modulator is ketanserin and the psychedelic is 2C-B HCl Form C, wherein the ketanserin is administered to post-treat at least 300 minutes after the 2C-B HCl Form C. In some embodiments, the serotonin receptor modulator is ketanserin and the psychedelic is 2C-B HCl Form C, wherein the ketanserin is administered to post-treat at least 330 minutes after the 2C-B HCl Form C. In some embodiments, the serotonin receptor modulator is ketanserin and the psychedelic is 2C-B HCl Form C, wherein the ketanserin is administered to post-treat at least 360 minutes after the 2C-B HCl Form C. In some preferred embodiments, the serotonin receptor modulator is ketanserin and the psychedelic is 2C-B HCl Form C, wherein ketanserin is administered to post-treat between about 60 minutes and about 180 minutes after the administration of the 2C-B HCl Form C.

In some embodiments, the serotonin receptor modulator is ritanserin and the psychedelic is 2C-B HCl Form C, wherein the ritanserin is administered to post-treat at least 15 minutes after the administration of the 2C-B HCl Form C. In some embodiments, the serotonin receptor modulator is ritanserin and the psychedelic is 2C-B HCl Form C, wherein the ritanserin is administered to post-treat at least 30 minutes after the 2C-B HCl Form C. In some embodiments, the serotonin receptor modulator is ritanserin and the psychedelic is 2C-B HCl Form C, wherein the ritanserin is administered to post-treat between at least 60 minutes and 240 minutes after the administration or release of the 2C-B HCl Form C. In some embodiments, the serotonin receptor modulator is ritanserin and the psychedelic is 2C-B HCl Form C, wherein the ritanserin is administered to post-treat at least 90 minutes after the 2C-B HCl Form C. In some embodiments, the serotonin receptor modulator is ritanserin and the psychedelic is 2C-B HCl Form C, wherein the ritanserin is administered to post-treat at least 120 minutes after the 2C-B HCl Form C. In some embodiments, the serotonin receptor modulator is ritanserin and the psychedelic is 2C-B HCl Form C, wherein the ritanserin is administered to post-treat at least 150 minutes after the 2C-B HCl Form C. In some embodiments, the serotonin receptor modulator is ritanserin and the psychedelic is 2C-B HCl Form C, wherein the ritanserin is administered to post-treat between about 15 minutes and about 150 minutes after the 2C-B HCl Form C. In some embodiments, the serotonin receptor modulator is ritanserin and the psychedelic is 2C-B HCl Form C, wherein the ritanserin is administered to post-treat at least 180 minutes after the 2C-B HCl Form C. In some embodiments, the serotonin receptor modulator is ritanserin and the psychedelic is 2C-B HCl Form C, wherein the ritanserin is administered to post-treat at least 210 minutes after the 2C-B HCl Form C. In some embodiments, the serotonin receptor modulator is ritanserin and the psychedelic is 2C-B HCl Form C, wherein the ritanserin is administered to post-treat at least 240 minutes after the 2C-B HCl Form C. In some embodiments, the serotonin receptor modulator is ritanserin and the psychedelic is 2C-B HCl Form C, wherein the ritanserin is administered to post-treat at least 270 minutes after the 2C-B HCl Form C. In some embodiments, the serotonin receptor modulator is ritanserin and the psychedelic is 2C-B HCl Form C, wherein the ritanserin is administered to post-treat at least 300 minutes after the 2C-B HCl Form C. In some embodiments, the serotonin receptor modulator is ritanserin and the psychedelic is 2C-B HCl Form C, wherein the ritanserin is administered to post-treat at least 330 minutes after the 2C-B HCl Form C. In some embodiments, the serotonin receptor modulator is ritanserin and the psychedelic is 2C-B HCl Form C, wherein the ritanserin is administered to post-treat at least 360 minutes after the 2C-B HCl Form C. In some preferred embodiments, the serotonin receptor modulator is ritanserin and the psychedelic is 2C-B HCl Form C, wherein ritanserin is administered to post-treat between about 60 minutes and about 180 minutes after the administration of the 2C-B HCl Form C.

In some embodiments, the serotonin receptor modulator is pimavanserin and the psychedelic is 2C-B HCl Form C, wherein the pimavanserin is administered to post-treat at least 15 minutes after the administration of the 2C-B HCl Form C. In some embodiments, the serotonin receptor modulator is pimavanserin and the psychedelic is 2C-B HCl Form C, wherein the pimavanserin is administered to post-treat at least 30 minutes after the 2C-B HCl Form C. In some embodiments, the serotonin receptor modulator is pimavanserin and the psychedelic is 2C-B HCl Form C, wherein the pimavanserin is administered to post-treat between at least 60 minutes and 240 minutes after the administration or release of the 2C-B HCl Form C. In some embodiments, the serotonin receptor modulator is pimavanserin and the psychedelic is 2C-B HCl Form C, wherein the pimavanserin is administered to post-treat at least 90 minutes after the 2C-B HCl Form C. In some embodiments, the serotonin receptor modulator is pimavanserin and the psychedelic is 2C-B HCl Form C, wherein the pimavanserin is administered to post-treat at least 120 minutes after the 2C-B HCl Form C. In some embodiments, the serotonin receptor modulator is pimavanserin and the psychedelic is 2C-B HCl Form C, wherein the pimavanserin is administered to post-treat at least 150 minutes after the 2C-B HCl Form C. In some embodiments, the serotonin receptor modulator is pimavanserin and the psychedelic is 2C-B HCl Form C, wherein the pimavanserin is administered to post-treat between about 15 minutes and about 150 minutes after the 2C-B HCl Form C. In some embodiments, the serotonin receptor modulator is pimavanserin and the psychedelic is 2C-B HCl Form C, wherein the pimavanserin is administered to post-treat at least 180 minutes after the 2C-B HCl Form C. In some embodiments, the serotonin receptor modulator is pimavanserin and the psychedelic is 2C-B HCl Form C, wherein the pimavanserin is administered to post-treat at least 210 minutes after the 2C-B HCl Form C. In some embodiments, the serotonin receptor modulator is pimavanserin and the psychedelic is 2C-B HCl Form C, wherein the pimavanserin is administered to post-treat at least 240 minutes after the 2C-B HCl Form C. In some embodiments, the serotonin receptor modulator is pimavanserin and the psychedelic is 2C-B HCl Form C, wherein the pimavanserin is administered to post-treat at least 270 minutes after the 2C-B HCl Form C. In some embodiments, the serotonin receptor modulator is pimavanserin and the psychedelic is 2C-B HCl Form C, wherein the pimavanserin is administered to post-treat at least 300 minutes after the 2C-B HCl Form C. In some embodiments, the serotonin receptor modulator is pimavanserin and the psychedelic is 2C-B HCl Form C, wherein the pimavanserin is administered to post-treat at least 330 minutes after the 2C-B HCl Form C. In some embodiments, the serotonin receptor modulator is pimavanserin and the psychedelic is 2C-B HCl Form C, wherein the pimavanserin is administered to post-treat at least 360 minutes after the 2C-B HCl Form C. In some preferred embodiments, the serotonin receptor modulator is pimavanserin and the psychedelic is 2C-B HCl Form C, wherein pimavanserin is administered to post-treat between about 60 minutes and about 180 minutes after the administration of the 2C-B HCl Form C.

In some embodiments, the serotonin receptor modulator is nelotanserin and the psychedelic is 2C-B HCl Form C, wherein the nelotanserin is administered to post-treat at least 15 minutes after the administration of the 2C-B HCl Form C. In some embodiments, the serotonin receptor modulator is nelotanserin and the psychedelic is 2C-B HCl Form C, wherein the nelotanserin is administered to post-treat at least 30 minutes after the 2C-B HCl Form C. In some embodiments, the serotonin receptor modulator is nelotanserin and the psychedelic is 2C-B HCl Form C, wherein the nelotanserin is administered to post-treat between at least 60 minutes and 240 minutes after the administration or release of the 2C-B HCl Form C. In some embodiments, the serotonin receptor modulator is nelotanserin and the psychedelic is 2C-B HCl Form C, wherein the nelotanserin is administered to post-treat at least 90 minutes after the 2C-B HCl Form C. In some embodiments, the serotonin receptor modulator is nelotanserin and the psychedelic is 2C-B HCl Form C, wherein the nelotanserin is administered to post-treat at least 120 minutes after the 2C-B HCl Form C.

In some embodiments, the serotonin receptor modulator is nelotanserin and the psychedelic is 2C-B HCl Form C, wherein the nelotanserin is administered to post-treat at least 150 minutes after the 2C-B HCl Form C. In some embodiments, the serotonin receptor modulator is nelotanserin and the psychedelic is 2C-B HCl Form C, wherein the nelotanserin is administered to post-treat between about 15 minutes and about 150 minutes after the 2C-B HCl Form C. In some embodiments, the serotonin receptor modulator is nelotanserin and the psychedelic is 2C-B HCl Form C, wherein the nelotanserin is administered to post-treat at least 180 minutes after the 2C-B HCl Form C. In some embodiments, the serotonin receptor modulator is nelotanserin and the psychedelic is 2C-B HCl Form C, wherein the nelotanserin is administered to post-treat at least 210 minutes after the 2C-B HCl Form C. In some embodiments, the serotonin receptor modulator is nelotanserin and the psychedelic is 2C-B HCl Form C, wherein the nelotanserin is administered to post-treat at least 240 minutes after the 2C-B HCl Form C. In some embodiments, the serotonin receptor modulator is nelotanserin and the psychedelic is 2C-B HCl Form C, wherein the nelotanserin is administered to post-treat at least 270 minutes after the 2C-B HCl Form C.

In some embodiments, the serotonin receptor modulator is nelotanserin and the psychedelic is 2C-B HCl Form C, wherein the nelotanserin is administered to post-treat at least 300 minutes after the 2C-B HCl Form C. In some embodiments, the serotonin receptor modulator is nelotanserin and the psychedelic is 2C-B HCl Form C, wherein the nelotanserin is administered to post-treat at least 330 minutes after the 2C-B HCl Form C. In some embodiments, the serotonin receptor modulator is nelotanserin and the psychedelic is 2C-B HCl Form C, wherein the nelotanserin is administered to post-treat at least 360 minutes after the 2C-B HCl Form C. In some preferred embodiments, the serotonin receptor modulator is nelotanserin and the psychedelic is 2C-B HCl Form C, wherein nelotanserin is administered to post-treat between about 60 minutes and about 180 minutes after the administration of the 2C-B HCl Form C. In some embodiments, the serotonin receptor modulator is pruvanserin and the psychedelic is 2C-B HCl Form C, wherein the pruvanserin is administered to post-treat at least 15 minutes after the administration of the 2C-B HCl Form C. In some embodiments, the serotonin receptor modulator is pruvanserin and the psychedelic is 2C-B HCl Form C, wherein the pruvanserin is administered to post-treat at least 30 minutes after the 2C-B HCl Form C. In some embodiments, the serotonin receptor modulator is pruvanserin and the psychedelic is 2C-B HCl Form C, wherein the pruvanserin is administered to post-treat between at least 60 minutes and 240 minutes after the administration or release of the 2C-B HCl Form C. In some embodiments, the serotonin receptor modulator is pruvanserin and the psychedelic is 2C-B HCl Form C, wherein the pruvanserin is administered to post-treat at least 90 minutes after the 2C-B HCl Form C. In some embodiments, the serotonin receptor modulator is pruvanserin and the psychedelic is 2C-B HCl Form C, wherein the pruvanserin is administered to post-treat at least 120 minutes after the 2C-B HCl Form C. In some embodiments, the serotonin receptor modulator is pruvanserin and the psychedelic is 2C-B HCl Form C, wherein the pruvanserin is administered to post-treat at least 150 minutes after the 2C-B HCl Form C. In some embodiments, the serotonin receptor modulator is pruvanserin and the psychedelic is 2C-B HCl Form C, wherein the pruvanserin is administered to post-treat between about 15 minutes and about 150 minutes after the 2C-B HCl Form C. In some embodiments, the serotonin receptor modulator is pruvanserin and the psychedelic is 2C-B HCl Form C, wherein the pruvanserin is administered to post-treat at least 180 minutes after the 2C-B HCl Form C. In some embodiments, the serotonin receptor modulator is pruvanserin and the psychedelic is 2C-B HCl Form C, wherein the pruvanserin is administered to post-treat at least 210 minutes after the 2C-B HCl Form C.

In some embodiments, the serotonin receptor modulator is pruvanserin and the psychedelic is 2C-B HCl Form C, wherein the pruvanserin is administered to post-treat at least 240 minutes after the 2C-B HCl Form C. In some embodiments, the serotonin receptor modulator is pruvanserin and the psychedelic is 2C-B HCl Form C, wherein the pruvanserin is administered to post-treat at least 270 minutes after the 2C-B HCl Form C. In some embodiments, the serotonin receptor modulator is pruvanserin and the psychedelic is 2C-B HCl Form C, wherein the pruvanserin is administered to post-treat at least 300 minutes after the 2C-B HCl Form C. In some embodiments, the serotonin receptor modulator is pruvanserin and the psychedelic is 2C-B HCl Form C, wherein the pruvanserin is administered to post-treat at least 330 minutes after the 2C-B HCl Form C. In some embodiments, the serotonin receptor modulator is pruvanserin and the psychedelic is 2C-B HCl Form C, wherein the pruvanserin is administered to post-treat at least 360 minutes after the 2C-B HCl Form C. In some preferred embodiments, the serotonin receptor modulator is pruvanserin and the psychedelic is 2C-B HCl Form C, wherein pruvanserin is administered to post-treat between about 60 minutes and about 180 minutes after the administration of the 2C-B HCl Form C.

In some embodiments, the serotonin receptor modulator is flibanserin, wherein the flibanserin is administered to post-treat at least 15 minutes post to the administration of the 2C-B HCl Form C. In some embodiments, the serotonin receptor modulator is flibanserin, wherein the flibanserin is administered to post-treat at least 30 minutes post to the 2C-B HCl Form C. In some embodiments, the serotonin receptor modulator is flibanserin, wherein the flibanserin is administered to post-treat between at least 60 minutes and 240 minutes post to the administration or release of the 2C-B HCl Form C. In some embodiments, the serotonin receptor modulator is flibanserin, wherein the flibanserin is administered to post-treat at least 90 minutes post to the 2C-B HCl Form C. In some embodiments, the serotonin receptor modulator is flibanserin, wherein the flibanserin is administered to post-treat at least 120 minutes post to the 2C-B HCl Form C. In some embodiments, the serotonin receptor modulator is flibanserin, wherein the flibanserin is administered to post-treat at least 150 minutes post to the 2C-B HCl Form C. In some embodiments, the serotonin receptor modulator is flibanserin, wherein the flibanserin is administered to post-treat between about 15 minutes and about 150 minutes post to the 2C-B HCl Form C. In some embodiments, the serotonin receptor modulator is flibanserin, wherein the flibanserin is administered to post-treat at least 180 minutes post to the 2C-B HCl Form C. In some embodiments, the serotonin receptor modulator is flibanserin, wherein the flibanserin is administered to post-treat at least 210 minutes post to the 2C-B HCl Form C.

In some embodiments, the serotonin receptor modulator is flibanserin, wherein the flibanserin is administered to post-treat at least 240 minutes post to the 2C-B HCl Form C. In some embodiments, the serotonin receptor modulator is flibanserin, wherein the flibanserin is administered to post-treat at least 270 minutes post to the 2C-B HCl Form C. In some embodiments, the serotonin receptor modulator is flibanserin, wherein the flibanserin is administered to post-treat at least 300 minutes post to the 2C-B HCl Form C. In some embodiments, the serotonin receptor modulator is flibanserin, wherein the flibanserin is administered to post-treat at least 330 minutes post to the 2C-B HCl Form C. In some embodiments, the serotonin receptor modulator is flibanserin, wherein the flibanserin is administered to post-treat at least 360 minutes post to the 2C-B HCl Form C. In some preferred embodiments, the serotonin receptor modulator is flibanserin, wherein flibanserin is administered to post-treat between about 60 minutes and about 180 minutes post to the administration of the 2C-B HCl Form C.

In some embodiments, the serotonin receptor modulator is olanzapine, wherein the olanzapine is administered to post-treat at least 15 minutes post to the administration of the 2C-B HCl Form C. In some embodiments, the serotonin receptor modulator is olanzapine, wherein the olanzapine is administered to post-treat at least 30 minutes post to the 2C-B HCl Form C. In some embodiments, the serotonin receptor modulator is olanzapine, wherein the olanzapine is administered to post-treat between at least 60 minutes and 240 minutes post to the administration or release of the 2C-B HCl Form C. In some embodiments, the serotonin receptor modulator is olanzapine, wherein the olanzapine is administered to post-treat at least 90 minutes post to the 2C-B HCl Form C. In some embodiments, the serotonin receptor modulator is olanzapine, wherein the olanzapine is administered to post-treat at least 120 minutes post to the 2C-B HCl Form C. In some embodiments, the serotonin receptor modulator is olanzapine, wherein the olanzapine is administered to post-treat at least 150 minutes post to the 2C-B HCl Form C. In some embodiments, the serotonin receptor modulator is olanzapine, wherein the olanzapine is administered to post-treat between about 15 minutes and about 150 minutes post to the 2C-B HCl Form C. In some embodiments, the serotonin receptor modulator is olanzapine, wherein the olanzapine is administered to post-treat at least 180 minutes post to the 2C-B HCl Form C. In some embodiments, the serotonin receptor modulator is olanzapine, wherein the olanzapine is administered to post-treat at least 210 minutes post to the 2C-B HCl Form C.

In some embodiments, the serotonin receptor modulator is olanzapine, wherein the olanzapine is administered to post-treat at least 240 minutes post to the 2C-B HCl Form C. In some embodiments, the serotonin receptor modulator is olanzapine, wherein the olanzapine is administered to post-treat at least 270 minutes post to the 2C-B HCl Form C. In some embodiments, the serotonin receptor modulator is olanzapine, wherein the olanzapine is administered to post-treat at least 300 minutes post to the 2C-B HCl Form C. In some embodiments, the serotonin receptor modulator is olanzapine, wherein the olanzapine is administered to post-treat at least 330 minutes post to the 2C-B HCl Form C. In some embodiments, the serotonin receptor modulator is olanzapine, wherein the olanzapine is administered to post-treat at least 360 minutes post to the 2C-B HCl Form C. In some preferred embodiments, the serotonin receptor modulator is olanzapine, wherein olanzapine is administered to post-treat between about 60 minutes and about 180 minutes post to the administration of the 2C-B HCl Form C.

In some embodiments, the serotonin receptor modulator is quetiapine, wherein the quetiapine is administered to post-treat at least 15 minutes post to the administration of the 2C-B HCl Form C. In some embodiments, the serotonin receptor modulator is quetiapine, wherein the quetiapine is administered to post-treat at least 30 minutes post to the 2C-B HCl Form C. In some embodiments, the serotonin receptor modulator is quetiapine, wherein the quetiapine is administered to post-treat between at least 60 minutes and 240 minutes post to the administration or release of the 2C-B HCl Form C. In some embodiments, the serotonin receptor modulator is quetiapine, wherein the quetiapine is administered to post-treat at least 90 minutes post to the 2C-B HCl Form C. In some embodiments, the serotonin receptor modulator is quetiapine, wherein the quetiapine is administered to post-treat at least 120 minutes post to the 2C-B HCl Form C. In some embodiments, the serotonin receptor modulator is quetiapine, wherein the quetiapine is administered to post-treat at least 150 minutes post to the 2C-B HCl Form C. In some embodiments, the serotonin receptor modulator is quetiapine, wherein the quetiapine is administered to post-treat between about 15 minutes and about 150 minutes post to the 2C-B HCl Form C. In some embodiments, the serotonin receptor modulator is quetiapine, wherein the quetiapine is administered to post-treat at least 180 minutes post to the 2C-B HCl Form C. In some embodiments, the serotonin receptor modulator is quetiapine, wherein the quetiapine is administered to post-treat at least 210 minutes post to the 2C-B HCl Form C.

In some embodiments, the serotonin receptor modulator is quetiapine, wherein the quetiapine is administered to post-treat at least 240 minutes post to the 2C-B HCl Form C. In some embodiments, the serotonin receptor modulator is quetiapine, wherein the quetiapine is administered to post-treat at least 270 minutes post to the 2C-B HCl Form C. In some embodiments, the serotonin receptor modulator is quetiapine, wherein the quetiapine is administered to post-treat at least 300 minutes post to the 2C-B HCl Form C. In some embodiments, the serotonin receptor modulator is quetiapine, wherein the quetiapine is administered to post-treat at least 330 minutes post to the 2C-B HCl Form C. In some embodiments, the serotonin receptor modulator is quetiapine, wherein the quetiapine is administered to post-treat at least 360 minutes post to the 2C-B HCl Form C. In some preferred embodiments, the serotonin receptor modulator is quetiapine, wherein quetiapine is administered to post-treat between about 60 minutes and about 180 minutes post to the administration of the 2C-B HCl Form C.

In some embodiments, the serotonin receptor modulator is risperidone, wherein the risperidone is administered to post-treat at least 15 minutes post to the administration of the 2C-B HCl Form C. In some embodiments, the serotonin receptor modulator is risperidone, wherein the risperidone is administered to post-treat at least 30 minutes post to the 2C-B HCl Form C. In some embodiments, the serotonin receptor modulator is risperidone, wherein the risperidone is administered to post-treat between at least 60 minutes and 240 minutes post to the administration or release of the 2C-B HCl Form C. In some embodiments, the serotonin receptor modulator is risperidone, wherein the risperidone is administered to post-treat at least 90 minutes post to the 2C-B HCl Form C. In some embodiments, the serotonin receptor modulator is risperidone, wherein the risperidone is administered to post-treat at least 120 minutes post to the 2C-B HCl Form C. In some embodiments, the serotonin receptor modulator is risperidone, wherein the risperidone is administered to post-treat at least 150 minutes post to the 2C-B HCl Form C. In some embodiments, the serotonin receptor modulator is risperidone, wherein the risperidone is administered to post-treat between about 15 minutes and about 150 minutes post to the 2C-B HCl Form C. In some embodiments, the serotonin receptor modulator is risperidone, wherein the risperidone is administered to post-treat at least 180 minutes post to the 2C-B HCl Form C. In some embodiments, the serotonin receptor modulator is risperidone, wherein the risperidone is administered to post-treat at least 210 minutes post to the 2C-B HCl Form C.

In some embodiments, the serotonin receptor modulator is risperidone, wherein the risperidone is administered to post-treat at least 240 minutes post to the 2C-B HCl Form C. In some embodiments, the serotonin receptor modulator is risperidone, wherein the risperidone is administered to post-treat at least 270 minutes post to the 2C-B HCl Form C. In some embodiments, the serotonin receptor modulator is risperidone, wherein the risperidone is administered to post-treat at least 300 minutes post to the 2C-B HCl Form C. In some embodiments, the serotonin receptor modulator is risperidone, wherein the risperidone is administered to post-treat at least 330 minutes post to the 2C-B HCl Form C. In some embodiments, the serotonin receptor modulator is risperidone, wherein the risperidone is administered to post-treat at least 360 minutes post to the 2C-B HCl Form C. In some preferred embodiments, the serotonin receptor modulator is risperidone, wherein risperidone is administered to post-treat between about 60 minutes and about 180 minutes post to the administration of the 2C-B HCl Form C.

In some embodiments, the present compound forms act as non-hallucinogenic 5-HT_{2A} modulators (e.g., 5-HT_{2A} agonists) that are used to treat neurological diseases. In some embodiments, the present 2C-B forms act as non-hallucinogenic 5-HT_{2A} modulators (e.g., 5-HT_{2A} agonists) that are used for increasing neuronal plasticity. In some embodiments, the present 2C-B forms act as non-hallucinogenic 5-HT_{2A} modulators (e.g., 5-HT_{2A} agonists) that are used for increasing at least one of translation, transcription, or secretion of neurotrophic factors.

In some embodiments, increasing neuronal plasticity by treating a subject with a disclosed compound form can treat neurodegenerative disorder, Alzheimer's, Parkinson's disease, psychological disorder, depression, addiction, anxiety, post-traumatic stress disorder, treatment resistant depression, suicidal ideation, major depressive disorder, bipolar disorder, schizophrenia, stroke, traumatic brain injury, or substance use disorder.

In some embodiments, the present disclosure provides methods for increasing neuronal plasticity, comprising contacting a neuronal cell with a compound form of the present disclosure.

In some embodiments, a compound form disclosed herein.

In some embodiments, the methods described herein are for treating a disease or disorder that is a neurological disease. For example, a compound provided herein can exhibit, anti-addictive properties, antidepressant properties, anxiolytic properties, or a combination thereof. In some embodiments, the neurological disease is a neuropsychiatric disease. In some embodiments, the neuropsychiatric disease is a mood or anxiety disorder. In some embodiments, the neurological disease is a migraine, headaches (e.g., cluster headache), post-traumatic stress disorder (PTSD), anxiety, depression, neurodegenerative disorder, Alzheimer's disease, Parkinson's disease, psychological disorder, treatment resistant depression, suicidal ideation, major depressive disorder, bipolar disorder, schizophrenia, stroke, hypoxic brain injury, Chronic traumatic encephalopathy (CTE), traumatic brain injury, dementia, and addiction (e.g., substance use disorder). In some embodiments, the neurological disease is a migraine or cluster headache. In some embodiments, the neurological disease is a neurodegenerative disorder, dementia, Alzheimer's disease, or Parkinson's disease. In some embodiments, the neurological disease is dementia. In some embodiments, the neurological disease is a psychological disorder, treatment resistant depression, suicidal ideation, major depressive disorder, bipolar disorder, schizophrenia, post-traumatic stress disorder (PTSD), addiction (e.g., substance use disorder), depression, or anxiety.

In some embodiments, the neuropsychiatric disease is a psychological disorder, treatment resistant depression, suicidal ideation, major depressive disorder, bipolar disorder, schizophrenia, post-traumatic stress disorder (PTSD), addiction (e.g., substance use disorder), depression, or anxiety. In some embodiments, the neuropsychiatric disease or neurological disease is post-traumatic stress disorder (PTSD), addiction (e.g., substance use disorder), schizophrenia, depression, or anxiety. In some embodiments, the neuropsychiatric disease or neurological disease is addiction (e.g., substance use disorder). In some embodiments, the neuropsychiatric disease or neurological disease is depression. In some embodiments, the neuropsychiatric disease or neurological disease is anxiety. In some embodiments, the neuropsychiatric disease or neurological disease is post-traumatic stress disorder (PTSD). In some embodiments, the neurological disease is stroke or traumatic brain injury. In some embodiments, the neuropsychiatric disease or neurological disease is schizophrenia. In one embodiment of such therapy a form of 2C-B described herein is administered in combination with a serotonin receptor modulator. In certain embodiments the serotonin receptor modulator is selected from the group consisting of altanserin, blonanserin, eplivanserin, glemanserin, volinanserin, ketanserin, ritanserin, pimavanserin, nelotanserin, pruvanserin, and flibanserin. In one embodiment, the serotonin receptor modulator is selected from the group consisting of serotonin receptor modulator is selected from the group consisting of eplivanserin, volinanserin, ketanserin, ritanserin, pimavanserin, nelotanserin, pruvanserin, flibanserin, olanzapine, quetiapine, and risperidone.

### EXAMPLES

### Example 1—Polymorph Production

The active pharmaceutical ingredient (API), 4-bromo-2,5-dimethoxyphenethylamine hydrochloride, is characterized to evaluate its physical properties. The evaluation is performed by X-ray powder diffraction (XRPD), polarized light microscopy (PLM), differential scanning calorimetry (DSC), thermogravimetry (TG), dynamic vapor sorption/desorption (DVS), and/or solubility testing in organic solvents, water, and mixed solvent systems. XRPD data are used to assess crystallinity. PLM data are used to evaluate crystallinity and particle size/morphology. DSC data are used to evaluate melting point, thermal stability, and crystalline form conversion. TG data are used to evaluate if the API is a solvate or hydrate, and to evaluate thermal stability. DVS data are used to evaluate hygroscopicity of the API and if hydrates can be formed at high relative humidity.

XRPD characterization of crystalline 2C-B.HCl herein resulted in the XRPD patterns illustrated in FIGS. 1 and 2. The form characterized in FIGS. 1 and 2 is designated as Form A herein. TGA of the Form A material showed only a negligible weight loss. A sharp endotherm at 238° C. in the DSC thermogram is attributed to melting. Based on DVS data, Form A is slightly hygroscopic but loses all the gained moisture without form change.

To assess solubility, about 10 to 15 solvents may be selected from the list below, based on their properties (polarity, dielectric constant and dipole moment).

TABLE 1
Solvents

| | |
|---|---|
| acetic acid | n-heptane |
| acetone | n-hexane |
| acetonitrile | 1,1,1,3,3,3-hexafluoro-2-propanol |
| benzyl alcohol | isobutanol (2-methyl-1-propanol) |
| 1-butanol | isopentanol (3-methyl-1-butanol) |
| 2-butanol | isopropyl alcohol (2-propanol) |
| butyl acetate | isopropylbenzene (cumene) |
| t-butyl methyl ether | methanol |
| chlorobenzene | methoxybenzene (anisole) |
| chloroform | methyl acetate |
| di(ethylene glycol) | methyl ethyl ketone (2-butanone) |
| dichloromethane | methyl isobutyl ketone |
| diethyl ether | nitromethane |
| diethylamine | N-methyl-2-pyrrolidone (NMP) |
| Dimethylacetamide (DMA) | 1-octanol |
| diisopropyl ether | 1-pentanol |
| N,N-dimethyl-formamide (DMF) | 1-propanol |
| dimethyl sulfoxide | perfluorohexane |
| 1,4-dioxane | propyl acetate |
| 1,2-ethanediol (ethylene glycol) | 1,1,2,2-tetrachloroethane |
| ethanol | tetrahydrofuran |
| ethanolamine | toluene |
| 2-ethoxyethanol (Cellusolve) | 1,1,1-trichloroethane |
| ethyl acetate | 2,2,2-trifluoroethanol |
| ethyl formate | water |
| formic acid | o-xylene (1,2-dimethylbenzene) |
| glycerol | p-xylene (1,4-dimethylbenzene) |

The information obtained is used for designing the subsequent polymorph screen. Solvents are used as a single solvent or as solvent mixtures, some containing water.

As above, Form A was further assessed for solubility: The experiments were carried out by adding the test solvent in aliquots to weighed portions of solid. Whether dissolution had occurred was judged by visual inspection after addition of each solvent aliquot. The results are shown in Table 2. Solubility was calculated by dividing the weight of the sample by the total amount of solvent used to dissolve the sample. The actual solubilities may be greater than the numbers calculated because of the use of solvent aliquots that were too large or because of slow dissolution rates. All solubility measurements were carried out at room temperature unless noted otherwise. Solubility of 2C-B.HCl estimated as described above are provided in Table 2.

TABLE 2

| | Solubility |
|---|---|
| Solvent | (mg/mL) |
| Acetone | <2 |
| Acetone/H₂O 80/20 | >79 |
| ACN | <1 |
| EtOAc | <2 |
| EtOH | 13 |
| H₂O | 34 |
| IPA | <2 |
| MeOH | 41 |
| MeOH/CHCl₃ 50/50 | 41 |
| THF | <2 |

(a) ACN = acetonitrile; H₂O = water; EtOAc = ethyl acetate; EtOH = ethanol; IPA = isopropanol; MeOH = methanol; CHCl₃ = chloroform; THF = tetrahydrofuran

The techniques used for the polymorph screen are chosen based on the solvent selected and properties of the API. The following techniques (or a combination of techniques) may be used for the polymorph screening;
API is dissolved in a solvent or mixture of solvents, and the solvents are evaporated at different rates (slow evaporation or fast evaporation) and at different temperatures (ambient or elevated).
API is dissolved in a solvent or mixture of solvents (at ambient temperature or an elevated temperature), and the final solution is cooled (between −78° C. to 20° C.). The cooling method can be a fast cooling (by plunging the sample to an ice bath or a dry ice/acetone bath), or slow cooling. The solids formed will be recovered by filtration and dried (air dried or vacuum dried).
API is dissolved in a solvent or mixture of solvents, and an antisolvent is added to precipitate the salt. The solids formed will be recovered by filtration and dried (air dried or vacuum dried).
API is added to a solvent or mixture of solvents, where the API is not fully dissolved. The slurry will be agitated at different temperatures for a number of days. The solids formed will be recovered by filtration and (air dried or vacuum dried).
API is milled (by mechanical milling or by mortar and pestle), with a drop of solvent, or without any solvent.
API is melted and cooled (at different cooling rates, fast and slow, and cooled to different temperatures) to obtain solids.
API is suspended in a solvent or mixture of solvents, and the slurry is placed in a heating/cooling cycle for multiple cycles. The remaining solids after the final cooling cycle will be filtered and (air dried or vacuum dried).
API is processed to obtain an amorphous form (by melting, milling, solvent evaporation, spray drying or lyophilization). The amorphous form will then be exposed to elevated humidity (or elevated temperature, or combination thereof), or to solvent vapors for extended period of days.
API is exposed to elevated humidity (or elevated temperature, or combination thereof), or to solvent vapors for extended period of days.
Two or more polymorphs of the API are mixed in a solvent or solvent systems (some solvent mixtures containing variable amount of water) to obtain a slurry, and the slurry will be agitated (at various temperatures) for an extended period of time (days). The solvent system used can be pre-saturated with the API. The final solids will be filtered and dried (air dried or vacuum dried).
API is heated to a specific temperature and cooled (at ambient conditions or in a dry box).

The solids obtained are analyzed by XRPD to determine if they are crystalline and, if so, by DSC to see the melting point and by TG to see if they are hydrated/solvated, and by ¹H NMR spectroscopy to ensure chemical integrity. KF water titration is performed on forms that are hydrated. DVS analysis is performed to evaluate hygroscopicity of the form and if hydrated form is present. In particular variable temperature analyses, including variable temperature XRPD, are performed to assess the stability of each physical form as well as its crystallinity. Consistent

Consistent with the polymorph screen described above, 2C-B.HCl was mixed with various solvents under various conditions in attempts to generate polymorphs. The results from samples generated and analyzed are summarized in Table 3.

TABLE 3
Samples Generated and Analyzed

| | | | XRPD |
|---|---|---|---|
| Method | Solvent^{a} | Conditions^{b} | Results^{c} |
| Evaporation | Acetone/H₂O | Foil w/1 pinhole, RT | A |
| | EtOH | Foil w/1 pinhole, RT | B, may |
| | | | contain A |
| | | | (PO) |
| | H₂O | Foil w/1 pinhole, RT | C |
| | MeOH | Foil w/1 pinhole, RT | B, may |
| | | | contain A |
| | | | (PO) |
| | MeOH/CHCl₃ | Foil w/1 pinhole, RT | A + B |
| | | | (PO) |
| | PrOH | Foil w/1 pinhole, RT | B, may |
| | | | contain A |
| | | | (PO) |
| Precipitation | EtOH/heptane | 60° C. [symbol] 5° C. | B (PO) |
| | EtOH/hexanes | 60° C. [symbol] 5° C. | A + B |
| | EtOH/IPE | 60° C. [symbol] 5° C. | A |
| | EtOH/iPrOAc | 60° C. [symbol] 5° C. | A |
| | EtOH/MTBE | 60° C. [symbol] 5° C. | A |
| | H₂O/ACN | RT [symbol] 5° C.; NS. | — |
| | H₂O/dioxane | RT [symbol] 5° C.; NS. | B, may |
| | | Partial E, RT [symbol] 5° C. | contain A |
| | | | (PO) |
| | H₂O/PrOH | RT [symbol] 5° C.; NS. | — |
| | | Partial E, RT [symbol] 5° C., | |
| | | NS. | |
| | MeOH/DCM | RT [symbol] 5° C.; NS. | A + C |
| | | Partial E, RT [symbol] 5° C. | (PO) |
| | MeOH/DEE | RT | A |
| | MeOH/iPrOAc | RT [symbol] 5° C. | A |
| | MeOH/MIBK | RT [symbol] 5° C. | A |
| | MeOH/MTBE | RT [symbol] 5° C. | A + B |
| | PrOH/DEE | RT | A + B |
| Slurry | Acetone/DMF 91/9 | RT, 2 weeks | A |
| | ACN/H₂O 99/1 | RT, 2 weeks | A |
| | CHCl₃ | RT, 2 weeks | A |
| | DCM/H₂O 98/2 | RT, 2 weeks | C |
| | EtOAc (wet) | RT, 2 weeks | C |
| | EtOH | RT, 2 weeks | A |
| Slurry | IPA | RT, 2 weeks | A |
| | iPrOAc/DMF 91/9 | RT, 2 weeks | A |
| | MEK/MeOH 77/23 | RT, 2 weeks | A |
| | MTBE/MeOH 86/14 | RT, 2 weeks | A |
| Cooling | Acetone | 50° C. [symbol] 5° C.; NS. | A |
| | | Partial E, RT [symbol] 5° C. | |
| | ACN/H₂O 98/2 | 50° C. [symbol] 5° C.; NS. | A + pk |
| | | Partial E, RT [symbol] 5° C. | |
| | CHCl₃/MeOH | 60° C. [symbol] 5° C., NS. | — |
| | EtOAc/MeOH 89/11 | 50° C. [symbol] 5° C. | A (PO) |
| | | | A (rendered |
| | | | sample and |
| | | | reanalyzed) |
| | Hexanes/EtOH | 60° C. [symbol] 5° C. | A + B |
| | IPA | 50° C. [symbol] 5° C. | B |
| | | | B (FIG. 41; |
| | | | Table 57) |
| | IPE/MeOH | 60° C. [symbol] RT | A (PO) |
| | MIBK/MeOH | 60° C. [symbol] 5° C., NS. | — |
| | MTBE/MeOH | 60° C. [symbol] RT | A + B |
| | | | (PO) |
| | THF/H₂O 98/2 | 50° C. [symbol] 5° C.; NS. | A (PO) |
| | | Partial E, RT [symbol] 5° C. | |
| | Toluene/MeOH | 60° C. [symbol] 5° C. | A (PO) |
| Vapor | H₂O | A/S: Acetone; NS. | A + C |
| Diffusion | | Transferredto fridge; | |
| | | NS. E, RT | |
| | H₂O | A/S: ACN; NS. | A + trace C |
| | | Transferred tofridge; | |
| | | NS. E, RT | |
| | H₂O | A/S: THF; NS. | — |
| | | RT [symbol] 5° C.; NS. E, | |
| | | RT (gummy solids). | |
| | MeOH | A/S: DEE | A + B |
| | | | (PO) |
| | MeOH | A/S: EtOAc | A + B |
| | | | (PO) |
| | MeOH | A/S: | B + A |
| | | Hexanes; NS. | |
| | | Transferred | |
| | | to fridge. | |
| | MeOH | A/S: MTBE | B, may |
| | | | contain A |
| | | | (PO) |

^{a}ACN = acetonitrile; H₂O = water; EtOAc = ethyl acetate; EtOH = ethanol; IPA = isopropanol; MeOH = methanol; PrOH = 1-propanol; CHCl₃ = chloroform; IPE = di-isopropyl ether; MIBK = methyl iso-butyl ketone; MTBE = methyl tert-butyl ether; iPrOAc = isopropyl acetate; MEK = methyl ethyl ketone; DCM = dichloromethane; DEE = diethyl ether; DMF = dimethyl formamide;
^{b}NS = no solids; E = evaporation; RT = room/ambient temperature;
^{c}PO = preferred orientation
^{a}ACN = acetonitrile; H₂O = water; EtOAc = ethyl acetate; EtOH = ethanol; IPA = isopropanol; MeOH = methanol; CHCl₃ = chloroform; THF = tetrahydrofuran; IPE = di-isopropyl ether; MIBK = methyl iso-butyl ketone; MTBE = methyl tert-butyl ether; iPrOAc = isopropyl acetate; MEK = methyl ethyl ketone; DEE = diethyl ether; DMF = dimethyl formamide;
^{b}NS = no solids; E = evaporation; A/S = anti-solvent; RT = room/ambient temperature;
^{c}PO = preferred orientation; pk = peak

2C-B free base was also used as an alternative starting material. This was done in order to do in-situ experiments with hydrochloric acid as these types of experiments may provide access to polymorphs of the HCl salt that would not otherwise be obtained. Salt breaking experiments are described in Table 4 and in-situ experiments for the polymorph screen are summarized in Table 5.

TABLE 4

| | XRPD |
|---|---|
| Conditions^{a} | Results |
| Added 343 μL of 1N NaOH to a solution of 101.5 mg | FB 1 + 2; LC |
| of 2C-B HCl in ~5-6 mL water (cloudy, then oiling | |
| observed). Extracted w/DCM and EtOAc. Combined | |
| organic layers and dried w/MgSO4. | |
| Evaporated w/stream of air. | |
| Added 1,091 μL of 1N NaOH to a solution of | |
| 215.8 mg of 2C-B HCl in ~3-4 mL water (oil). | |
| Stirring, RT, 30 minutes. Extracted w/EtOAc (3x). | |
| Combined organic layers and dried w/MgSO₄. | |
| Evaporated w/stream of air. | |
| Added 15.2 mL of 1N NaOH to a solution of 3.0 g of | FB 1; LC |
| 2C-B HCl in ~60 mL water (oil). Stirring, RT, 1 day. | |
| Extracted w/EtOAc (3x). Combined organic layers | |
| and dried w/MgSO₄. | |
| Evaporated w/stream of air. | |

^{a}NaOH = sodium hydroxide; DCM = dichloromethane; EtOAc = ethyl acetate; MgSO₄ = magnesium sulfate; FB = free base

TABLE 5

| | | | XRPD |
|---|---|---|---|
| Method | Solvent | Conditions^{a} | Results |
| Slurry | ACN | RT, 6 days | A |
| | DCE | RT, 6 days | A |
| | Dioxane | RT, 6 days | A |
| | Heptane/EtOH | RT, 6 days | A |
| | IPA | RT, 6 days | A + B |
| | IPE | 60° C., 1 day [symbol] RT, | A |
| | | 5 days | |
| | iPrOAc | RT, 6 days | A |
| | 2-Me THF | RT, 6 days | A |
| | MEK | RT, 6 days | A |
| | MTBE | 60° C., 1 day [symbol] RT, | A + trace C |
| | | 5 days | |

^{a}ACN = acetonitrile; DCE = dichloroethane; EtOH = ethanol; IPA = isopropanol; IPE = di-isopropyl ether; iPrOAc = isopropyl acetate; 2-Me THF = 2-methyl tetrahydrofuran; MEK = methyl ethyl ketone; MTBE = methyl tert-butyl ether

In certain embodiments crystalline forms a prepared in a preferred orientation. Preferred orientation can increase or decrease peak intensities such that the peaks seem to appear and/or disappear between different patterns of the same form. An example of an XRPD diffractogram of Form A in preferred orientation is provided in FIG. 7.

As illustrated in the data provide above, two polymorphs of 2C-B.HCl have been identified in addition to Form A. They were designated as Form B (FIG. 3) and Form C (FIG. 4). Form B has only been observed from alcohol-containing solvent systems, and was typically isolated as a mixture with Form A. Form C has mostly been obtained from aqueous systems.

In some embodiments, the characterization data of 2C-B.HCl (Form A) are as provided in FIG. 1 and Table 6.

TABLE 6
XRPD Signal angle data of 2C-B•HCl (Form A)

| Position | d-value | Relative |
|---|---|---|
| 5.7 | 15.61 | 100 |
| 13.0 | 6.83 | 13 |
| 16.2 | 5.46 | 29 |
| 16.9 | 5.25 | 52 |
| 17.4 | 5.10 | 27 |
| 19.6 | 4.52 | 8 |
| 20.6 | 4.32 | 36 |
| 21.0 | 4.24 | 13 |
| 21.7 | 4.10 | 13 |
| 23.1 | 3.85 | 49 |
| 23.4 | 3.80 | 60 |
| 24.1 | 3.70 | 95 |
| 24.9 | 3.58 | 19 |
| 25.5 | 3.49 | 28 |
| 25.9 | 3.44 | 34 |
| 26.8 | 3.32 | 10 |
| 27.5 | 3.24 | 35 |
| 28.4 | 3.14 | 10 |
| 29.0 | 3.08 | 13 |
| 29.3 | 3.05 | 17 |
| 29.8 | 3.00 | 15 |
| 30.7 | 2.91 | 42 |
| 31.6 | 2.83 | 18 |
| 33.0 | 2.71 | 12 |
| 34.2 | 2.62 | 32 |
| 35.9 | 2.50 | 11 |
| 36.2 | 2.48 | 12 |
| 37.2 | 2.42 | 17 |
| 39.0 | 2.31 | 14 |

In some embodiments, the characterization data of 2C-B.HCl (Form A) are as provided in FIG. 2 and Table 7.

TABLE 7
XRPD Signal angle data of 2C-B-HCl (Form A)

| Position | d-value | Relative |
|---|---|---|
| 5.6 | 15.78 | 68 |
| 12.9 | 6.85 | 15 |
| 16.2 | 5.48 | 33 |
| 16.8 | 5.26 | 63 |
| 17.4 | 5.11 | 39 |
| 19.6 | 4.53 | 15 |
| 20.5 | 4.33 | 39 |
| 21.0 | 4.24 | 22 |
| 21.6 | 4.11 | 21 |
| 23.0 | 3.86 | 51 |
| 23.4 | 3.80 | 84 |
| 24.0 | 3.70 | 100 |
| 24.8 | 3.60 | 26 |
| 25.4 | 3.50 | 32 |
| 25.9 | 3.45 | 41 |
| 27.4 | 3.25 | 39 |
| 28.3 | 3.15 | 17 |
| 28.9 | 3.09 | 23 |
| 29.3 | 3.05 | 25 |
| 29.7 | 3.00 | 20 |
| 30.7 | 2.92 | 43 |
| 31.6 | 2.83 | 25 |
| 33.0 | 2.71 | 21 |
| 34.1 | 2.63 | 36 |
| 35.0 | 2.56 | 16 |
| 36.1 | 2.49 | 21 |
| 37.1 | 2.42 | 24 |
| 39.0 | 2.31 | 20 |

In some embodiments, the characterization data of 2C-B.HCl (Form B) are as provided in FIG. 3 and Table 8.

TABLE 8
XRPD Signal angle data of 2C-B•HCl (Form B)

| Position | d-value | Relative |
|---|---|---|
| 5.0 | 17.74 | 4 |
| 5.5 | 16.01 | 100 |
| 16.0 | 5.53 | 3 |
| 16.6 | 5.35 | 21 |
| 17.4 | 5.10 | 2 |
| 20.3 | 4.37 | 3 |
| 21.5 | 4.13 | 2 |
| 22.1 | 4.02 | 3 |
| 22.6 | 3.94 | 3 |
| 23.0 | 3.87 | 4 |
| 23.4 | 3.80 | 4 |
| 23.8 | 3.74 | 4 |
| 24.3 | 3.66 | 2 |
| 25.1 | 3.55 | 3 |
| 25.3 | 3.53 | 3 |
| 25.9 | 3.44 | 3 |
| 27.0 | 3.30 | 3 |
| 27.5 | 3.25 | 3 |
| 27.8 | 3.21 | 3 |
| 28.9 | 3.09 | 2 |
| 30.2 | 2.96 | 9 |
| 31.6 | 2.83 | 3 |
| 33.5 | 2.68 | 5 |
| 34.0 | 2.64 | 5 |
| 35.6 | 2.52 | 2 |
| 36.1 | 2.49 | 2 |
| 36.9 | 2.44 | 3 |
| 37.2 | 2.42 | 2 |
| 38.4 | 2.35 | 2 |
| 38.8 | 2.32 | 3 |
| 39.3 | 2.29 | 3 |

In some embodiments, the characterization data of 2C-B.HCl (Form C) are as provided in FIG. 4 and Table 9.

TABLE 9
XRPD Signal angle data of 2C-B•HCl (Form C)

| Position | d-value | Relative |
|---|---|---|
| 4.5 | 19.46 | 100 |
| 9.0 | 9.78 | 15 |
| 13.1 | 6.77 | 9 |
| 13.6 | 6.53 | 31 |
| 14.0 | 6.32 | 5 |
| 15.3 | 5.81 | 11 |
| 16.7 | 5.31 | 5 |
| 18.3 | 4.84 | 15 |
| 19.7 | 4.51 | 6 |
| 20.2 | 4.40 | 8 |
| 20.8 | 4.26 | 6 |
| 21.1 | 4.21 | 6 |
| 21.7 | 4.10 | 10 |
| 21.9 | 4.05 | 15 |
| 22.7 | 3.92 | 22 |
| 23.3 | 3.81 | 6 |
| 23.9 | 3.73 | 10 |
| 24.8 | 3.59 | 6 |
| 25.1 | 3.55 | 13 |
| 25.5 | 3.50 | 10 |
| 25.9 | 3.45 | 82 |
| 26.3 | 3.39 | 12 |
| 26.9 | 3.31 | 15 |
| 27.3 | 3.27 | 19 |
| 27.8 | 3.21 | 11 |
| 28.4 | 3.14 | 8 |
| 28.8 | 3.10 | 18 |
| 29.5 | 3.03 | 6 |
| 30.0 | 2.97 | 13 |
| 30.8 | 2.90 | 7 |
| 31.1 | 2.87 | 8 |
| 31.7 | 2.82 | 8 |
| 32.2 | 2.78 | 6 |
| 34.4 | 2.61 | 35 |
| 34.9 | 2.57 | 9 |
| 36.1 | 2.49 | 7 |
| 36.7 | 2.45 | 25 |
| 37.6 | 2.39 | 7 |
| 37.8 | 2.38 | 8 |
| 38.6 | 2.33 | 11 |
| 38.9 | 2.31 | 7 |

In some embodiments, the characterization data of 2C-B (free base forms 1 and 2) are as provided in FIGS. 5 and 36 and Table 10.

TABLE 10
XRPD Signal angle data of 2C-B (free base forms 1 and 2)

| Position | d-value | Relative |
|---|---|---|
| 5.4 | 16.24 | 60 |
| 9.2 | 9.57 | 48 |
| 15.3 | 5.78 | 32 |
| 16.3 | 5.44 | 41 |
| 17.0 | 5.21 | 81 |
| 17.6 | 5.04 | 43 |
| 18.6 | 4.77 | 46 |
| 20.2 | 4.40 | 45 |
| 21.7 | 4.10 | 55 |
| 22.5 | 3.95 | 32 |
| 23.6 | 3.77 | 99 |
| 24.3 | 3.66 | 100 |
| 25.3 | 3.52 | 78 |
| 26.6 | 3.36 | 54 |
| 27.1 | 3.29 | 43 |
| 28.2 | 3.17 | 51 |
| 29.8 | 3.00 | 28 |
| 31.2 | 2.87 | 29 |
| 32.3 | 2.77 | 36 |
| 33.0 | 2.71 | 32 |
| 33.9 | 2.65 | 39 |
| 34.9 | 2.57 | 33 |
| 36.3 | 2.47 | 38 |
| 37.1 | 2.42 | 42 |
| 37.8 | 2.38 | 42 |
| 39.3 | 2.29 | 34 |

In some embodiments, the characterization data of 2C-B (free base form 1) are as provided in FIGS. 6 and 28 and Table 11.

TABLE 11
XRPD Signal angle data of 2C-B (free base form 1)

| Position | d-value | Relative |
|---|---|---|
| 5.3 | 16.67 | 37 |
| 10.5 | 8.41 | 27 |
| 13.8 | 6.40 | 27 |
| 15.3 | 5.79 | 47 |
| 16.0 | 5.54 | 57 |
| 16.6 | 5.35 | 35 |
| 17.6 | 5.05 | 88 |
| 19.0 | 4.68 | 53 |
| 20.6 | 4.30 | 46 |
| 21.0 | 4.23 | 43 |
| 21.4 | 4.16 | 42 |
| 21.8 | 4.08 | 57 |
| 22.4 | 3.97 | 43 |
| 22.8 | 3.90 | 49 |
| 23.3 | 3.82 | 71 |
| 24.2 | 3.68 | 75 |
| 24.4 | 3.65 | 84 |
| 25.3 | 3.52 | 100 |
| 26.2 | 3.41 | 54 |
| 26.5 | 3.36 | 52 |
| 26.9 | 3.32 | 71 |
| 27.8 | 3.21 | 43 |
| 28.8 | 3.10 | 38 |
| 30.3 | 2.95 | 27 |
| 31.4 | 2.85 | 30 |
| 32.1 | 2.79 | 29 |
| 32.9 | 2.73 | 32 |
| 33.5 | 2.68 | 30 |
| 34.4 | 2.61 | 26 |
| 35.1 | 2.56 | 34 |
| 36.4 | 2.47 | 34 |
| 37.9 | 2.37 | 30 |
| 38.6 | 2.33 | 32 |

In some embodiments, the characterization data of 2C-B.HCl (Form A) are as provided in FIG. 7 and Table 12.

TABLE 12
XRPD Signal angle data of 2C-B•HCl (Form A)

| Position | d-value | Relative |
|---|---|---|
| 5.6 | 15.67 | 100 |
| 11.3 | 7.84 | 1 |
| 16.9 | 5.24 | 40 |
| 20.6 | 4.31 | 0 |
| 22.6 | 3.93 | 2 |
| 23.3 | 3.81 | 0 |
| 24.1 | 3.70 | 0 |
| 25.5 | 3.49 | 1 |
| 28.4 | 3.14 | 4 |
| 30.8 | 2.90 | 2 |
| 34.2 | 2.62 | 9 |
| 36.3 | 2.48 | 0 |
| 37.1 | 2.42 | 0 |

Form B and Form C were further characterized by NMR, TGA, and DSC and the data are summarized in Table 13.

TABLE 13

| | Analytical | |
|---|---|---|
| Sample | Technique | Results^{a} |
| Form B | ¹H NMR | Consistent with structure |
| sample | | No organic solvents |
| | TGA | 0.4% start to 144° C. |
| | DSC | Endo: 240° C. (onset 239° C., ΔH°_{fus} = 122.3 J/g) |
| Form C | ¹H NMR | Consistent with structure |
| sample | | No organic solvents |
| | TGA | 4.2% start to 86° C. |
| | DSC | Endo: 73° C., 238° C. (onset 236° C., |
| | | ΔH°_{fus} = 105.8 J/g) |
| FB 1 | ¹H NMR | Consistent with structure |
| sample | | Significant peak shifts observed for peaks |
| | | attributable to methylene protons |
| | | Small unidentified peaks at 3.1 and 2.6 ppm |

^{a}¹H NMR = proton nuclear magnetic resonance spectroscopy; TGA = thermogravimetric analysis; DSC = differential scanning calorimetry; endo = endotherm; exo = exotherm; FB = free base

Based on the data, Form B appears to be anhydrous as there are no organic solvents observed by proton NMR and the TG thermogram shows only a small weight loss (0.4%). A sharp endotherm present at 240° C. by DSC for Form B is typical of melting.

Form C is suspected to be hydrated. The TG thermogram shows a 4.2% weight loss from ambient to 86° C., which is accompanied by a broad endotherm in the DSC thermogram. Because there are no organic solvents observed in the proton NMR spectrum, this is attributed to water and would be equivalent to approximately 0.5 moles. The sharp endotherm at 238° C. in the DSC thermogram for Form C is typical of melting.

Form A was found to be more thermodynamically stable than Form B. That relationship was established from results of competitive slurry experiments. Typically, competitive slurry experiments are conducted by suspending mixtures of different forms in a solvent pre-saturated with the compound of interest. The more stable form is expected to result from the initial mixture of forms by dissolution-recrystallization.

For these experiments, mixtures of Form A and B were slurried in solvents pre-saturated with 2C-B.HCl. Note that the slurries were carried out at both ambient and elevated temperature because the relationship can change with changes in temperature. Experiments and results are described in Table 14.

TABLE 14

| | | XRPD |
|---|---|---|
| Solvent | Conditions^{a} | Results |
| EtOAc | Slurry, RT | A |
| EtOH | Slurry, RT | A |
| IPA | Slurry, RT | A |
| MEK | Slurry, 50° C. | A |
| MTBE/MeOH | Slurry, RT | A |

Water activity studies were conducted to determine the boundary between Form A and Form C. For these experiments, 2C-B.HCl Form A was suspended in solvents containing different amounts of water, thus having different water activities. The slurries were carried out at ambient temperature and, like with relative stability, the water activity boundary can change with temperature. The results are summarized in Table 15.

TABLE 15

| | | XRPD |
|---|---|---|
| Solvent | Conditions | Results |
| ACN/H₂O 99/1 | Slurry, RT, 7 days. Solids | A |
| a_{w} = 0.21 | analyzed wet. | |
| THF/H₂O 98/2 | Slurry, RT, 7 days. Solids | A |
| a_{w} = 0.38 | analyzed wet. | |
| ACN/H₂O 97/3 | Slurry, RT, 7 days. Solids | A |
| a_{w} = 0.50 | analyzed wet. | |
| ACN/H₂O 96/4 | Slurry, RT, 6 days. Solids | C |
| a_{w} = 0.60 | analyzed wet. | |
| ACN/H₂O 94/6 | Slurry, RT, 6 days. Solids | C |
| a_{w} = 0.73 | analyzed wet. | |
| THF/H₂O 94/6 | Slurry, RT, 6 days. Solids | C |
| a_{w} = 0.84 | analyzed wet. | |
| THF/H₂O 90/10 | Slurry, RT, 6 days. Solids | C |
| a_{w} = 1.00 | analyzed wet. | |

a_{w} = water activity (predicted from solvent system) With reference to Table 8, Form C was obtained at water activities of 0.6 and above. Form A was observed at water activities of 0.5 and below. This means that the boundary of conversion between the two forms is between 0.5 and 0.6 water activity.

Instrumental Techniques

The data summarized above were gathered as follows:

For X-ray Powder Diffraction (XRPD), a Rigaku Smart-Lab X-ray diffraction system was configured for reflection Bragg-Brentano geometry using a line source X-ray beam. The X-ray source is a Cu Long Fine Focus tube that was operated at 40 kV and 44 ma. That source provides an incident beam profile at the sample that changes from a narrow line at high angles to a broad rectangle at low angles. Beam conditioning slits are used on the line X-ray source to ensure that the maximum beam size is less than 10 mm both along the line and normal to the line. The Bragg-Brentano geometry is a para-focusing geometry controlled by passive divergence and receiving slits with the sample itself acting as the focusing component for the optics. The inherent resolution of Bragg-Brentano geometry is governed in part by the diffractometer radius and the width of the receiving slit used. Typically, the Rigaku Smart-Lab is operated to give peak widths of 0.1 °2θ or less. The axial divergence of the X-ray beam is controlled by 5.0-degree Soller slits in both the incident and diffracted beam paths.

Powder samples were prepared in a low background Si holder using light manual pressure to keep the sample surfaces flat and level with the reference surface of the sample holder. Each sample was analyzed from 2 to 40 °2θ using a continuous scan of 6 °2θ per minute with an effective step size of 0.02 °2θ.

Differential Scanning calorimetry (DSC) analyses were carried out using a TA Instruments Q2500 Discovery Series instrument. The instrument temperature calibration was performed using indium. The DSC cell was kept under a nitrogen purge of ˜50 mL per minute during each analysis. The sample was placed in a standard, crimped, aluminum pan and was heated from approximately 25° C. to 350° C. at a rate of 10° C. per minute.

Thermogravimetric (TG) analysis was carried out using a TA Instruments Q5500 Discovery Series instrument. The instrument balance was calibrated using class M weights and the temperature calibration was performed using alumel. The nitrogen purge was ˜40 mL per minute at the balance and ˜60 mL per minute at the furnace. Each sample was placed into a pre-tared platinum pan and heated from approximately 25° C. to 350° C. at a rate of 10° C. per minute.

Dynamic Vapor Sorption (DVS) analysis was carried out using a TA Instruments Q5000 Dynamic Vapor Sorption analyzer. The instrument was calibrated with standard weights and a sodium bromide standard for humidity. Approximately 10-25 mg of sample was loaded into a metal-coated quartz pan for analysis. The sample was analyzed at 25° C. with a maximum equilibration time of one hour in 10% relative humidity (RH) steps from 5 to 95% RH (adsorption cycle) and from 95 to 5% RH (desorption cycle). The movement from one step to the next occurred either after satisfying the equilibrium criterion of 0.01% weight change or, if the equilibrium criterion was not met, after one hour. The percent weight change values were calculated using Microsoft Excel.

Nuclear Magnetic Resonance (1H NMR) spectra were acquired on a Bruker Avance II 400 spectrometer. Samples were prepared by dissolving material in DMSO-d6. The solutions were filtered and placed into individual 5-mm NMR tubes for subsequent spectral acquisition. The temperature controlled (295K) 1H NMR spectra acquired on the Avance II 400 utilized a 5-mm cryoprobe operating at an observing frequency of 400.18 MHz.

Infrared (IR) spectra were obtained on a Nicolet 6700 FT-IR system. Samples were analyzed using a Nicolet SMART iTR attenuated total reflectance device.

Optical microscopy experiments were carried out on a Leica DM 2500 P compound microscope. Images were captured using a Qlmaging MicroPublisher 3.3 RTV camera. Images were collected at 10× magnification.

Alternative instrumental techniques employed may be the following: Differential scanning calorimetry (DSC) thermograms are obtained using a DSC Q 100 (TA Instruments, New Castle, Del.). The temperature axis and cell constant of the DSC cell are calibrated with indium (10 mg, 99.9% pure, melting point 156.6° C., heat of fusion 28.4 J/g). Samples (2.0-5.0 mg) are weighed in aluminum pans on an analytical balance. Aluminum pans without lids are used for the analysis. The samples are equilibrated at 25° C. and heated to 250-300° C. at a heating rate of 10° C./min under continuous nitrogen flow. TG analysis of the samples is performed with a Q 50 (TA Instruments, New Castle, Del.). Samples (2.0-5.0 mg) are analyzed in open aluminum pans under a nitrogen flow (50 mL/min) at 25° C. to 210° C. with a heating rate of 10° C./min.

The sample for moisture analysis is allowed to dry at 25° C. for up to 4 hours under a stream of dry nitrogen. The relative humidity is then increased stepwise from 10 to 90% relative humidity (adsorption scan) allowing the sample to equilibrate for a maximum of four hours before weighing and moving on to the next step. The desorption scan is measured from 85 to 0% relative humidity with the same equilibration time. The sample is then dried under a stream of dry nitrogen at 80° C. for 2 hours or until no weight loss is observed.

X-ray powder diffraction data are collected using a Miniflex Tabletop XRD system (Rigaku/MSC, The Woodlands, Tex.) from 5° to 45 °2θ with steps of 0.1°, and the measuring time is 1.0 second/step. All samples are ground to similar size before exposure to radiation. The powder samples are illuminated using CuKα radiation (λ=1.54056 Å) at 30 kV and 15 mA.

Variable temperature XRPD data are collected using a Huber Imaging Plate Guinier Camera 670 employing Ni-filtered CuKα₁ radiation (λ=1.5405981 Å) produced at 40 kV and 20 mA by a Philips PW1120/00 generator fitted with a Huber long fine-focus tube PW2273/20 and a Huber Guinier Monochromator Series 611/15. The original powder is packed into a Lindemann capillary (Hilgenberg, Germany) with an internal diameter of 1 mm and a wall thickness of 0.01 mm. The sample is heated at an average rate of 5 Kmin⁻¹ using a Huber High Temperature Controller HTC 9634 unit with the capillary rotation device 670.2. The temperature is held constant at selected intervals for 10 min while the sample is exposed to X-rays and multiple scans were recorded. A 20-range of 4.00-100.0° is used with a step size of 0.005 °2θ.

In certain embodiments wherein the solid form is a solvate, such as a hydrate, the DSC thermogram reveals endothermic transitions. In accordance with the observed DSC transitions, TGA analysis indicates stages of weight change corresponding to desolvation or dehydration and/or melting of the sample. In the case of hydrates, these results are in harmony with Karl Fisher titration data which indicate the water content of the sample.

The moisture sorption profile of a sample can be generated to assess the stability of a solid form is stable over a range of relative humidities. In certain embodiments, the change in moisture content over 10.0 to 95.0% relative humidity is small. In other embodiments the change in moisture content over 10.0 to 95.0% relative humidity is reversible.

In certain embodiments, the XRPD pattern of a sample of solid form indicates that the sample has a well-defined crystal structure and a high degree of crystallinity.

### Example 2—Salt Screen

4-bromo-2,5-dimethoxyphenethylamine is characterized to evaluate its physical properties. The evaluation is performed by X-ray powder diffraction (XRPD), polarized light microscopy (PLM), differential scanning calorimetry (DSC), thermogravimetry (TG), dynamic vapor sorption/desorption (DVS), and/or solubility testing in organic solvents, water, and mixed solvent systems. XRPD data are used to assess crystallinity. PLM data are used to evaluate crystallinity and particle size/morphology. DSC data are used to evaluate melting point, thermal stability, and crystalline form conversion. TG data are used to evaluate if the free base is a solvate or hydrate, and to evaluate thermal stability. DVS data are used to evaluate hygroscopicity of the free base and if hydrates can be formed at high relative humidity. About to 15 solvents are selected from the list below, based on their properties (polarity, dielectric constant and dipole moment).

TABLE 16
Solvents

| | |
|---|---|
| acetic acid | n-heptane |
| Acetone | n-hexane |
| Acetonitrile | 1,1,1,3,3,3-hexafluoro-2- |
| | propanol |
| benzyl alcohol | isobutanol (2-methyl-1- |
| | propanol) |
| 1-butanol | isopentanol (3-methyl-1- |
| | butanol) |
| 2-butanol | isopropyl alcohol (2-propanol) |
| butyl acetate | isopropylbenzene (cumene) |
| t-butyl methyl ether | Methanol |
| Chlorobenzene | methoxybenzene (anisole) |
| Chloroform | methyl acetate |
| di(ethylene glycol) | methyl ethyl ketone |
| | (2-butanone) |
| Dichloromethane | methyl isobutyl ketone |
| diethyl ether | Nitromethane |
| Diethylamine | N-methyl-2-pyrrolidone (NMP) |
| Dimethylacetamide (DMA) | 1-octanol |
| diisopropyl ether | 1-pentanol |
| N,N-dimethyl-formamide | 1-propanol |
| (DMF) | |
| dimethyl sulfoxide | Perfluorohexane |
| 1,4-dioxane | propyl acetate |
| 1,2-ethanediol (ethylene | 1,1,2,2-tetrachloroethane |
| glycol) | |
| Ethanol | Tetrahydrofuran |
| Ethanolamine | Toluene |
| 2-ethoxyethanol (Cellusolve) | 1,1,1-trichloroethane |
| ethyl acetate | 2,2,2-trifluoroethanol |
| ethyl formate | Water |
| formic acid | o-xylene (1,2-dimethylbenzene) |
| Glycerol | p-xylene (1,4-dimethylbenzene) |

The information obtained is used for designing the subsequent salt screen. The salt screen is performed by reacting the free base with pharmaceutically acceptable acids under various conditions in attempts to generate crystalline salts. Pharmaceutically acceptable acids that may be used are listed below. Specific acids are selected based on the pKa of the free base, and typically 15 to 20 acids are selected. Experiments are performed using 0.5 molar equivalent, 1 molar equivalent and/or 2 molar equivalents of the acid.

TABLE 17
Exemplary Acids

| | |
|---|---|
| naphthalene-1,5-disulfonic acid | citric acid |
| sulfuric acid | d-glucuronic acid |
| ethane-1,2-disulfonic acid | lactobionic acid |
| p-toluenesulfonic acid | D-glucoheptonic acid |
| thiocyanic acid | (−)-L-pyroglutamic acid |
| methanesulfonic acid | L-malic acid |
| dodecylsulfuric acid | hippuric acid |
| naphthalene-2-sulfonic acid | D-gluconic acid |
| benzenesulfonic acid | D,L-lactic acid |
| oxalic acid | oleic acid |
| glycerophosphoric acid | succinic acid |
| ethanesulfonic acid, 2-hydroxy | glutaric acid |
| L-aspartic acid | cinnamic acid |
| maleic acid | adipic acid |
| phosphoric acid | sebacic acid |
| ethanesulfonic acid | (+)-camphoric acid |
| glutamic acid | acetic acid |
| pamoic (embonic) acid | nicotinic acid |
| glutaric acid, 2-oxo- | isobutyric acid |
| 2-naphthoic acid, 1-hydroxy | propionic acid |
| malonic acid | lauric acid |
| gentisic acid | stearic acid |
| L-tartaric acid | orotic acid |
| fumaric acid | carbonic acid |
| galactaric (mucic) acid | |

Solvent systems for the salt crystallization experiments are selected based on the solubility of the free base and the selected acid. Solvents are used as a single solvent or as solvent mixtures, some containing water. The techniques that are used for salt crystallization are chosen based on the solvent selected and properties of the free base. The following techniques (or combination of techniques) may be used for salt crystallization;
Free base and acid are dissolved in a solvent or mixture of solvents, and the solvents are evaporated at different rates (slow evaporation or fast evaporation) and at different temperatures (ambient or elevated).
Free base and acid are dissolved in a solvent or mixture of solvents (at ambient temperature or an elevated temperature), and the final solution is cooled to a sub-ambient temperature (between −78° C. to 15° C.). The cooling method can be a fast cooling (by plunging the sample into an ice bath or a dry ice/acetone bath), or slow cooling. The solids formed will be recovered by filtration and dried (air dried or vacuum dried).
Free base and acid are dissolved in a solvent or mixture of solvents, and an antisolvent is added to precipitate the salt. The solids formed will be recovered by filtration and dried (air dried or vacuum dried).
Free base and acid are added to a solvent or mixture of solvents, where one or both components are not fully dissolved. The slurry is agitated at different temperatures for a number of days. The solids formed will be recovered by filtration and dried (air dried or vacuum dried). The same experiment can be also performed in solvent systems where the solvents are not miscible.
Free base and acid are milled together (by mechanical milling or by mortar and pestle), with a drop of solvent, or without any solvent.
Free base and acid are melted together, and cooled to various temperatures using various cooling rates.
If an amorphous form of a salt is obtained, the amorphous salt will be exposed to elevated humidity, or elevated temperature (or combination of both), or solvent vapors at various temperatures to form crystalline salts.

The stoichiometric ratio of acid to 4-bromo-2,5-dimethoxyphenethylamine is confirmed by ¹H NMR, HPLC, or both as is known to those of ordinary skill in the art.

The salts obtained are analyzed by XRPD to determine if they are crystalline and, if so, by DSC to see the melting point and by TG to see if they are hydrated/solvated, and by 1H NMR spectroscopy to ensure chemical integrity. KF water titration is performed on salts that are hydrated. DVS analysis is performed to evaluate hygroscopicity of the salt and if hydrated form is present.

2C-B free base to use in the salt screen was prepared as follows: Added 15.2 mL of 1N NaOH to a solution of 3.0 g of 2C-B HCl in ˜60 mL water (oil). Stirring, at room temperature for 1 day was followed by extraction with EtOAc (3×). Combined organic layers were dried with MgSO₄ and evaporated with a stream of air. The material was characterized by ¹H NMR (FIG. 20) and XRPD (FIG. 21).

An alternative preparation of 2C-B free base: 343 μL of 1N NaOH was added to a solution of 101.5 mg of 2C-B HCl in ˜5-6 mL water (cloudy, then oiling observed). The mixture was extracted with dichloromethane and ethyl acetate, and the combined organic layers were dried over MgSO₄. Drying with a stream of air yielded 2C-B free base as a solid. XRPD analysis of this material yielded the diffractogram provided in FIG. 29.

Screening of 2C-B free base with twenty-one acids was conducted by combining one equivalent acid with one equivalent 2C-B free base under the conditions outlined below in Table 18.

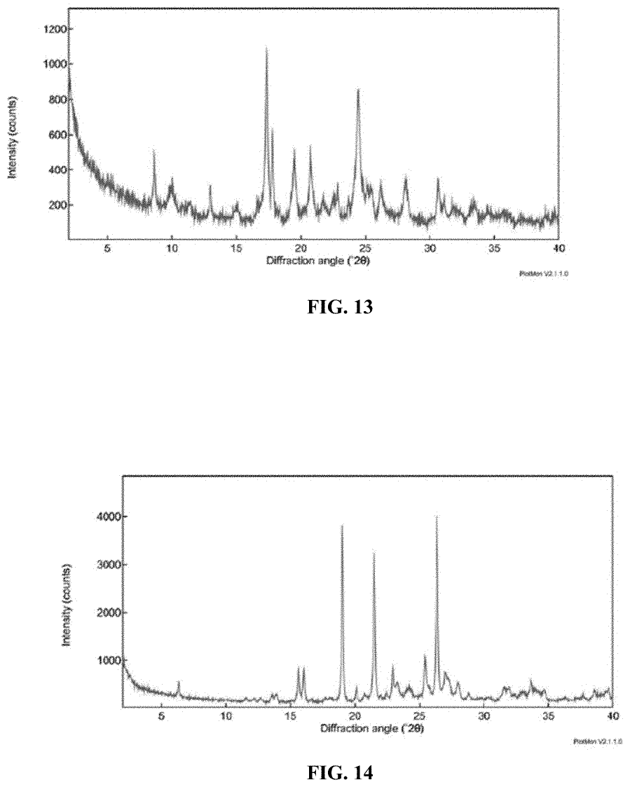

TABLE 18
Salt Screen Experiments using 2C-B free base

| Acid | Conditions^{a} | FIG. No. | XRPD Results^{b} |
|---|---|---|---|
| L-aspartic | Precipitation, EtOH/H₂O, 50° C.→RT; | FIG. 37 | Aspartate + |
| | No Solids. Stirred at 5° C.; No Solids. | | pks |
| | Evaporation RT | | |
| | Milling, MeOH/H₂O, 30 min | FIG. 37B | Aspartate + |
| | | | FB 1 |
| Benzenesulfonic | Precipitation, EtOH, RT | FIG. 8 | Besylate |
| | | | (Form 1) |
| | Precipitation, MEK, RT | FIG. 29 | Besylate |
| | | | (Form 2) |
| Citric | Slurry, EtOAc, 50° C. | FIG. 9 | Citrate |
| | | | (Form 1) |
| | Slurry, EtOAc, 50° C. 1 day, RT 5 days | FIG. 39 | Citrate |
| | | | (Form 2) |
| | Precipitation, MEK, 50° C.→RT | | Citrate |
| | | | (Form 1) |
| Ethanesulfonic | Precipitation, IPA, 50° C.→RT | FIG. 10 | Esylate 1 |
| (Esylate) | Precipitation, MTBE, 50° C.→RT | FIG. 30 | Esylate 2 |
| Fumaric | Slurry, IPE, 50° C. | FIG. 11 | Fumarate |
| | | | (Form 1) |
| | Precipitation, ACN, 50° C.→RT | | Fumarate |
| | | | (Form 1) |
| Gentisic | Precipitation, iPrOAc, 50° C. | FIG. 12 | Gentisate |
| | Slurry, ACN, 50° C., 1 d→RT | FIG. 56 | Gentisate |
| D-gluconic | Precipitation, IPA, RT | FIG. 13 | Gluconate* |
| | Precipitation, iPrOAc, 50° C.→RT | | degradant |
| L-glutamic | Precipitation, EtOH/FFO, 50° C.→RT; NS. | FIG. 58 | Glutamate |
| | Stirred at 5° C.; NS. Evaporation, RT | | |
| | Milling, MeOH/H₂O, 30 min | FIG. 38 | Glutamate |
| Glycolic | Precipitation, iPrOAc, 50° C. | FIG. 14 | Glycolate |
| | slurry, MTBE, 50° C., 1 d→RT | FIG. 59 | Glycolate |
| H₂SO₄ | Slurry, MTBE, 50° C., 4 d→RT | FIG. 15 | Sulfate |
| | Precipitation, IPA, RT | | Sulfate |
| H₃PO₄ | Slurry, MTBE, 50° C., 4 d→RT | FIG. 16 | Phosphate |
| | | | Form 1 + |
| | | | Form 2 |
| | Precipitation, IPA, 50° C. | FIG. 31 | Phosphate |
| | | | (Form 2) |
| 1-hydroxy 2- | Slurry, IPE, 50° C. | FIG. 17 | Xinafoate |
| napthoic | slurry, iPrOAc, RT | FIG. 63 | Xinafoate |
| (xinafoic) | | | |
| D,L-lactic | Precipitation, IPA, RT | FIG. 18 | Lactate |
| | | | (Form 1) |
| | Precipitation, iPrOAc, 50° C.→RT | FIG. 32 | Lactate |
| | | | (Form 2) |
| L-malic | Slurry, EtOAc, 50° C. | FIG. 19 | Malate + |
| | | | pks |
| | Precipitation, MEK, 50° C.→RT | | Malate + |
| | | | pks |
| Maleic | Slurry, IPE, 50° C. | FIG. 20 | Maleate |
| | | | (Form 1) |
| | Precipitation, MEK, RT | FIG. 33 | Maleate |
| | | | (Form 2) |
| Malonic | Slurry, EtOAc, 50° C., 4 d→RT | FIG. 21 | Malonate |
| | Precipitation, acetone, RT→5° C.; NS. | | Malonate + |
| | evaporation, RT; tacky solids. Vacuum- | | NC + pks |
| | dried, RT | | |
| Methanesulfonic | Slurry, MTBE, 50° C., 4 d→RT | FIG. 22 | Mesylate |
| | Precipitation, ACN, 50° C.→RT | | Mesylate |
| Mucic | Slurry, ACN, 50° C. | FIG. 23 | Mucate |
| | slurry, EtOH, 50° C., 3 d→RT | | Mucate |
| Succinic | Slurry, Acetone, RT | FIG. 24 | Succinate |
| | | | Form 1 and |
| | | | Form 2 + acid |
| | Precipitation IPA, | FIG. 34 | Succinate |
| | 50° C.→RT | | (Form 1) |
| L-tartaric | Slurry, Acetone, 50° C., 4 d→RT | FIG. 25 | Tartrate |
| | | | (Form 1) |
| | slurry, EtOH, RT | FIG. 64 | Tartrate |
| | | | (Form 1) |
| p-Toluenesulfonic | Slurry, EtOAc, RT | FIG. 26 | Tosylate |
| | | | (Form 1) |
| | Slurry, MTBE, | FIG. 35 | Tosylate |
| | 50° C., 1 d→RT | | (Forms 1 and |
| | | | 2) + acid |

^{a}EtOH = ethanol; MeOH = methanol; EtOAc = ethyl acetate; IPA = isopropanol; H₂O = water; IPE = di-isopropyl ether; iPrOAc = isopropyl acetate; MTBE = methyl tert-butyl ether; MEK = methyl ethyl ketone; ACN = acetonitrile; NS = no solids; RT = room/ambient temperature; d = day(s); min = minutes.
^{b}FB = 2C- B free base; pk(s) = peak(s); NC = non-crystalline.
*NMR analysis of the sample resulting from the combination of 2C-B free base with D-gluconic acid showed that it was composed of an unknown degradant or impurity, indicating salt formation of 2C-B gluconate did not occur.

In some embodiments, the characterization data of 2C-B besylate (Form 1) are as provided in FIG. 8 and Table 19.

TABLE 19
XRPD Signal angle data of 2C-B besylate (Form 1)

| Position (°2θ) | d-value | Relative |
|---|---|---|
| 8.4 | 10.55 | 19 |
| 11.5 | 7.69 | 9 |
| 12.1 | 7.29 | 8 |
| 13.0 | 6.83 | 5 |
| 13.4 | 6.59 | 85 |
| 15.5 | 5.70 | 74 |
| 16.4 | 5.40 | 15 |
| 16.7 | 5.30 | 20 |
| 16.9 | 5.25 | 35 |
| 18.3 | 4.85 | 10 |
| 18.5 | 4.80 | 20 |
| 19.2 | 4.62 | 14 |
| 20.7 | 4.30 | 8 |
| 21.1 | 4.21 | 29 |
| 21.6 | 4.11 | 4 |
| 21.9 | 4.05 | 26 |
| 22.5 | 3.94 | 64 |
| 23.0 | 3.86 | 49 |
| 23.7 | 3.75 | 32 |
| 24.1 | 3.69 | 9 |
| 24.4 | 3.65 | 100 |
| 24.9 | 3.58 | 9 |
| 25.0 | 3.56 | 30 |
| 25.5 | 3.49 | 58 |
| 25.7 | 3.46 | 45 |
| 26.1 | 3.41 | 38 |
| 26.4 | 3.38 | 12 |
| 26.6 | 3.35 | 10 |
| 26.7 | 3.33 | 9 |
| 27.0 | 3.30 | 6 |
| 27.4 | 3.26 | 11 |
| 27.6 | 3.23 | 18 |
| 27.8 | 3.21 | 7 |
| 28.0 | 3.19 | 17 |
| 28.5 | 3.13 | 5 |
| 29.1 | 3.07 | 12 |
| 29.4 | 3.03 | 28 |
| 29.8 | 3.00 | 20 |
| 30.4 | 2.94 | 8 |
| 30.6 | 2.92 | 8 |
| 31.0 | 2.89 | 18 |
| 31.3 | 2.86 | 13 |
| 31.5 | 2.84 | 5 |
| 32.4 | 2.76 | 17 |
| 32.5 | 2.75 | 12 |
| 32.9 | 2.73 | 18 |
| 33.3 | 2.69 | 4 |
| 33.6 | 2.67 | 6 |
| 34.1 | 2.63 | 5 |
| 34.6 | 2.59 | 6 |
| 34.8 | 2.58 | 6 |
| 35.4 | 2.54 | 6 |
| 35.6 | 2.52 | 8 |
| 37.0 | 2.43 | 11 |
| 37.3 | 2.41 | 14 |
| 37.5 | 2.40 | 19 |
| 38.2 | 2.35 | 11 |
| 38.4 | 2.34 | 25 |
| 38.6 | 2.33 | 29 |
| 39.6 | 2.28 | 6 |

In some embodiments, the characterization data of 2C-B Citrate (form 1) are as provided in FIG. 9 and Table 20.

TABLE 20
XRPD Signal angle data of 2C-B Citrate (form 1)

| Position (°2θ) | d-value | Relative |
|---|---|---|
| 5.6 | 15.67 | 14 |
| 11.0 | 8.06 | 16 |
| 11.2 | 7.87 | 19 |
| 12.8 | 6.93 | 17 |
| 13.4 | 6.59 | 23 |
| 13.7 | 6.47 | 64 |
| 14.2 | 6.22 | 30 |
| 16.9 | 5.26 | 22 |
| 17.4 | 5.08 | 11 |
| 17.9 | 4.96 | 7 |
| 19.6 | 4.52 | 6 |
| 20.2 | 4.40 | 40 |
| 20.6 | 4.31 | 38 |
| 21.0 | 4.23 | 10 |
| 21.4 | 4.14 | 16 |
| 21.7 | 4.10 | 18 |
| 22.0 | 4.03 | 21 |
| 22.2 | 4.01 | 18 |
| 22.4 | 3.97 | 37 |
| 22.5 | 3.95 | 94 |
| 22.7 | 3.91 | 36 |
| 23.9 | 3.73 | 15 |
| 24.3 | 3.67 | 14 |
| 24.7 | 3.60 | 100 |
| 25.3 | 3.52 | 10 |
| 25.8 | 3.45 | 21 |
| 26.6 | 3.35 | 7 |
| 26.9 | 3.31 | 9 |
| 27.5 | 3.25 | 12 |
| 27.9 | 3.20 | 24 |
| 28.8 | 3.10 | 9 |
| 29.7 | 3.01 | 19 |
| 30.0 | 2.98 | 12 |
| 30.4 | 2.94 | 8 |
| 30.7 | 2.91 | 8 |
| 31.0 | 2.88 | 9 |
| 31.8 | 2.82 | 14 |
| 32.6 | 2.75 | 18 |
| 32.7 | 2.74 | 20 |
| 33.2 | 2.70 | 10 |
| 33.6 | 2.66 | 15 |
| 33.8 | 2.65 | 14 |
| 34.0 | 2.63 | 10 |
| 35.0 | 2.57 | 7 |
| 35.2 | 2.55 | 10 |
| 35.4 | 2.54 | 10 |
| 36.7 | 2.45 | 20 |
| 37.4 | 2.41 | 8 |
| 37.7 | 2.38 | 11 |
| 37.9 | 2.38 | 11 |
| 38.9 | 2.31 | 11 |
| 40.0 | 2.26 | 11 |

In some embodiments, the characterization data of 2C-B Esylate (Form 1) are as provided in FIG. 10 and Table 21.

TABLE 21
XRPD Signal angle data of 2C-B Esylate (Form 1)

| Position (°2θ) | d-value | Relative |
|---|---|---|
| 9.3 | 9.51 | 100 |
| 10.0 | 8.81 | 54 |
| 14.5 | 6.11 | 25 |
| 15.2 | 5.84 | 15 |
| 15.9 | 5.57 | 23 |
| 17.4 | 5.10 | 39 |
| 18.0 | 4.92 | 16 |
| 18.7 | 4.75 | 69 |
| 19.2 | 4.62 | 27 |
| 19.5 | 4.56 | 23 |
| 20.2 | 4.40 | 26 |
| 20.6 | 4.31 | 17 |
| 20.7 | 4.30 | 16 |
| 21.9 | 4.05 | 38 |
| 22.0 | 4.04 | 26 |
| 22.8 | 3.90 | 36 |
| 23.1 | 3.85 | 31 |
| 23.2 | 3.84 | 27 |
| 23.6 | 3.77 | 99 |
| 23.7 | 3.75 | 93 |
| 24.1 | 3.69 | 19 |
| 24.6 | 3.61 | 21 |
| 24.8 | 3.59 | 41 |
| 25.2 | 3.54 | 18 |
| 26.0 | 3.43 | 14 |
| 26.8 | 3.33 | 23 |
| 26.9 | 3.31 | 18 |
| 27.7 | 3.23 | 25 |
| 28.2 | 3.16 | 30 |
| 29.2 | 3.06 | 16 |
| 29.5 | 3.02 | 13 |
| 29.6 | 3.02 | 13 |
| 30.2 | 2.96 | 20 |
| 30.4 | 2.94 | 23 |
| 30.7 | 2.91 | 20 |
| 31.2 | 2.87 | 12 |
| 31.9 | 2.81 | 13 |
| 32.9 | 2.72 | 13 |
| 33.1 | 2.70 | 12 |
| 33.2 | 2.70 | 12 |
| 33.9 | 2.64 | 15 |
| 34.5 | 2.60 | 20 |
| 35.3 | 2.55 | 18 |
| 35.9 | 2.50 | 17 |
| 36.6 | 2.46 | 14 |
| 37.3 | 2.41 | 19 |
| 37.9 | 2.38 | 28 |
| 38.7 | 2.32 | 16 |
| 39.1 | 2.30 | 22 |
| 39.5 | 2.28 | 14 |

In some embodiments, the characterization data of 2C-B Fumarate (Form 1) are as provided in FIG. 11 and Table 22.

TABLE 22
XRPD Signal angle data of 2C-B Fumarate (Form 1)

| Position (°2θ) | d-value | Relative |
|---|---|---|
| 7.1 | 12.38 | 33 |
| 11.3 | 7.80 | 15 |
| 11.4 | 7.75 | 15 |
| 14.6 | 6.08 | 67 |
| 15.6 | 5.67 | 32 |
| 17.0 | 5.22 | 17 |
| 17.7 | 5.01 | 17 |
| 18.0 | 4.92 | 16 |
| 20.0 | 4.45 | 15 |
| 20.6 | 4.31 | 16 |
| 21.4 | 4.16 | 60 |
| 21.7 | 4.10 | 39 |
| 22.9 | 3.89 | 100 |
| 24.0 | 3.71 | 21 |
| 24.5 | 3.63 | 33 |
| 24.9 | 3.58 | 46 |
| 25.3 | 3.51 | 38 |
| 26.3 | 3.39 | 21 |
| 27.3 | 3.27 | 21 |
| 27.6 | 3.23 | 20 |
| 28.9 | 3.09 | 33 |
| 31.1 | 2.88 | 15 |
| 31.8 | 2.81 | 21 |
| 33.0 | 2.71 | 18 |
| 35.6 | 2.52 | 16 |

In some embodiments, the characterization data of 2C-B gentisate are as provided in FIG. 12 and Table 23.

TABLE 23
XRPD Signal angle data of 2C-B Gentisate

| Position (°2θ) | d-value | Relative |
|---|---|---|
| 10.1 | 8.76 | 28 |
| 11.1 | 7.97 | 9 |
| 13.1 | 6.74 | 42 |
| 14.3 | 6.20 | 14 |
| 15.0 | 5.92 | 7 |
| 15.5 | 5.70 | 11 |
| 16.5 | 5.36 | 33 |
| 17.3 | 5.14 | 25 |
| 18.1 | 4.89 | 9 |
| 18.9 | 4.70 | 14 |
| 19.2 | 4.61 | 8 |
| 20.2 | 4.39 | 23 |
| 20.5 | 4.33 | 21 |
| 20.9 | 4.25 | 20 |
| 22.2 | 4.00 | 100 |
| 22.6 | 3.94 | 20 |
| 22.8 | 3.90 | 11 |
| 23.6 | 3.76 | 18 |
| 23.8 | 3.74 | 24 |
| 24.1 | 3.69 | 12 |
| 24.3 | 3.67 | 22 |
| 24.9 | 3.57 | 13 |
| 25.2 | 3.53 | 20 |
| 25.6 | 3.48 | 86 |
| 26.4 | 3.37 | 28 |
| 26.7 | 3.34 | 14 |
| 27.3 | 3.27 | 11 |
| 28.7 | 3.11 | 18 |
| 29.3 | 3.05 | 6 |
| 30.1 | 2.97 | 22 |
| 30.5 | 2.93 | 12 |
| 31.1 | 2.87 | 8 |
| 31.4 | 2.85 | 6 |
| 31.9 | 2.80 | 8 |
| 32.1 | 2.79 | 11 |
| 32.4 | 2.76 | 8 |
| 32.8 | 2.73 | 16 |
| 33.1 | 2.70 | 13 |
| 33.7 | 2.66 | 11 |
| 34.6 | 2.59 | 13 |
| 35.5 | 2.53 | 13 |
| 36.8 | 2.44 | 12 |
| 38.0 | 2.37 | 12 |

NMR analysis of the sample resulting from the combination of 2C-B free base with D-gluconic acid showed that it was composed of an unknown degradant or impurity, indicating salt formation of 2C-B Gluconate did not occur.

In some embodiments, the characterization data of 2C-B glycolate are as provided in FIG. 14 and Table 25.

TABLE 25
XRPD Signal angle data of 2C-B Glycolate

| Position (°2θ) | d-value | Relative |
|---|---|---|
| 6.3 | 13.94 | 14 |
| 11.5 | 7.67 | 6 |
| 12.2 | 7.23 | 5 |
| 12.7 | 6.99 | 6 |
| 13.6 | 6.52 | 7 |
| 13.9 | 6.37 | 8 |
| 15.6 | 5.67 | 21 |
| 16.0 | 5.53 | 21 |
| 16.7 | 5.32 | 5 |
| 17.7 | 5.01 | 6 |
| 18.2 | 4.87 | 6 |
| 19.0 | 4.67 | 95 |
| 20.1 | 4.42 | 11 |
| 20.7 | 4.28 | 8 |
| 21.5 | 4.13 | 81 |
| 22.0 | 4.05 | 7 |
| 22.4 | 3.97 | 9 |
| 22.9 | 3.88 | 22 |
| 23.3 | 3.82 | 14 |
| 24.2 | 3.67 | 12 |
| 25.4 | 3.50 | 28 |
| 25.8 | 3.46 | 10 |
| 26.4 | 3.38 | 100 |
| 27.0 | 3.31 | 19 |
| 27.2 | 3.27 | 15 |
| 28.0 | 3.19 | 14 |
| 28.8 | 3.10 | 8 |
| 29.8 | 3.00 | 6 |
| 30.4 | 2.94 | 6 |
| 31.6 | 2.83 | 11 |
| 31.9 | 2.80 | 11 |
| 32.3 | 2.77 | 7 |
| 33.0 | 2.71 | 8 |
| 33.7 | 2.66 | 14 |
| 34.7 | 2.59 | 9 |
| 36.3 | 2.47 | 6 |
| 37.1 | 2.42 | 5 |
| 37.7 | 2.39 | 6 |
| 38.6 | 2.33 | 9 |
| 39.6 | 2.28 | 10 |

In some embodiments, the characterization data of 2C-B sulfate are as provided in FIG. and Table 26.

TABLE 26
XRPD Signal angle data of 2C-B Sulfate

| Position (°2θ) | d-value | Relative |
|---|---|---|
| 8.7 | 10.16 | 53 |
| 13.0 | 6.79 | 47 |
| 15.1 | 5.88 | 16 |
| 16.4 | 5.40 | 19 |
| 18.0 | 4.94 | 35 |
| 19.6 | 4.52 | 11 |
| 19.9 | 4.47 | 21 |
| 20.4 | 4.36 | 28 |
| 21.0 | 4.24 | 24 |
| 21.4 | 4.16 | 14 |
| 21.8 | 4.08 | 41 |
| 22.7 | 3.91 | 11 |
| 23.2 | 3.83 | 65 |
| 23.9 | 3.73 | 11 |
| 25.1 | 3.55 | 100 |
| 25.7 | 3.47 | 76 |
| 26.2 | 3.40 | 23 |
| 26.5 | 3.36 | 13 |
| 26.7 | 3.33 | 13 |
| 27.0 | 3.30 | 9 |
| 27.8 | 3.21 | 28 |
| 28.1 | 3.18 | 12 |
| 28.5 | 3.13 | 38 |
| 29.0 | 3.07 | 20 |
| 29.3 | 3.05 | 15 |
| 29.7 | 3.00 | 10 |
| 30.3 | 2.95 | 6 |
| 31.4 | 2.85 | 7 |
| 31.9 | 2.81 | 25 |
| 32.9 | 2.72 | 12 |
| 33.1 | 2.70 | 17 |
| 33.6 | 2.67 | 7 |
| 34.1 | 2.63 | 17 |
| 34.7 | 2.58 | 9 |
| 35.0 | 2.57 | 14 |
| 35.8 | 2.51 | 6 |
| 36.8 | 2.44 | 16 |
| 37.2 | 2.42 | 7 |
| 37.4 | 2.40 | 9 |
| 37.7 | 2.38 | 10 |
| 39.1 | 2.30 | 7 |

In some embodiments, the characterization data of 2C-B Phosphate (Form 1 and Form 2) are as provided in FIG. 16 and Table 27.

TABLE 27
XRPD Signal angle data of 2C-B Phosphate (Form 1 and Form 2)

| Position (°2θ) | d-value | Relative |
|---|---|---|
| 4.3 | 20.45 | 67 |
| 9.6 | 9.19 | 23 |
| 9.8 | 9.06 | 23 |
| 13.6 | 6.52 | 24 |
| 13.9 | 6.38 | 73 |
| 15.0 | 5.91 | 17 |
| 15.7 | 5.63 | 38 |
| 18.2 | 4.87 | 68 |
| 18.5 | 4.81 | 83 |
| 18.8 | 4.71 | 77 |
| 19.3 | 4.59 | 31 |
| 19.6 | 4.53 | 30 |
| 20.5 | 4.32 | 24 |
| 20.8 | 4.26 | 17 |
| 21.7 | 4.10 | 26 |
| 21.9 | 4.05 | 51 |
| 22.5 | 3.96 | 34 |
| 22.8 | 3.89 | 32 |
| 23.0 | 3.86 | 52 |
| 23.2 | 3.83 | 41 |
| 23.9 | 3.72 | 100 |
| 24.4 | 3.65 | 65 |
| 24.7 | 3.61 | 47 |
| 24.9 | 3.58 | 69 |
| 25.3 | 3.53 | 79 |
| 25.4 | 3.50 | 37 |
| 25.7 | 3.47 | 56 |
| 26.2 | 3.40 | 63 |
| 26.3 | 3.39 | 57 |
| 26.5 | 3.36 | 74 |
| 26.9 | 3.31 | 39 |
| 27.1 | 3.30 | 32 |
| 27.3 | 3.27 | 26 |
| 27.7 | 3.22 | 25 |
| 27.9 | 3.19 | 64 |
| 28.2 | 3.16 | 31 |
| 28.4 | 3.14 | 20 |
| 28.8 | 3.10 | 50 |
| 29.0 | 3.07 | 28 |
| 30.3 | 2.95 | 17 |
| 31.0 | 2.88 | 17 |
| 31.5 | 2.84 | 26 |
| 31.8 | 2.82 | 26 |
| 32.4 | 2.76 | 31 |
| 33.4 | 2.69 | 22 |
| 33.9 | 2.64 | 24 |
| 35.2 | 2.55 | 34 |
| 35.8 | 2.51 | 19 |
| 36.0 | 2.49 | 19 |
| 37.2 | 2.42 | 27 |
| 37.7 | 2.39 | 17 |
| 38.5 | 2.34 | 19 |
| 39.4 | 2.29 | 20 |
| 39.6 | 2.27 | 21 |

In some embodiments, the characterization data of 2C-B xinafoate are as provided in FIG. 17 and Table 28.

TABLE 28
XRPD Signal angle data of 2C-B Xinafoate

| Position (°2θ) | d-value | Relative |
|---|---|---|
| 7.9 | 11.25 | 66 |
| 8.2 | 10.73 | 16 |
| 13.0 | 6.82 | 16 |
| 13.8 | 6.40 | 35 |
| 14.3 | 6.19 | 36 |
| 14.7 | 6.03 | 22 |
| 15.1 | 5.87 | 28 |
| 15.7 | 5.64 | 9 |
| 16.4 | 5.39 | 31 |
| 17.3 | 5.11 | 17 |
| 18.0 | 4.92 | 19 |
| 18.5 | 4.79 | 78 |
| 19.4 | 4.58 | 11 |
| 20.0 | 4.44 | 60 |
| 20.8 | 4.27 | 21 |
| 21.5 | 4.14 | 23 |
| 22.4 | 3.97 | 57 |
| 23.3 | 3.82 | 83 |
| 24.7 | 3.60 | 100 |
| 25.4 | 3.51 | 19 |
| 26.1 | 3.41 | 68 |
| 26.7 | 3.33 | 22 |
| 27.0 | 3.30 | 19 |
| 27.5 | 3.25 | 27 |
| 27.8 | 3.21 | 24 |
| 28.0 | 3.18 | 21 |
| 28.6 | 3.12 | 14 |
| 29.0 | 3.07 | 14 |
| 29.3 | 3.04 | 14 |
| 29.7 | 3.01 | 16 |
| 30.4 | 2.94 | 16 |
| 31.7 | 2.82 | 18 |
| 32.4 | 2.76 | 17 |
| 33.2 | 2.70 | 10 |
| 33.8 | 2.65 | 10 |
| 34.1 | 2.63 | 12 |
| 34.8 | 2.58 | 16 |
| 35.0 | 2.56 | 20 |
| 35.9 | 2.50 | 12 |
| 37.1 | 2.42 | 12 |
| 37.8 | 2.38 | 14 |
| 38.7 | 2.33 | 14 |

In some embodiments, the characterization data of 2C-B Lactate (Form 1) are as provided in FIG. 18 and Table 29.

TABLE 29
XRPD Signal angle data of 2C-B Lactate (Form 1)

| Position (°2θ) | d-value | Relative |
|---|---|---|
| 7.1 | 12.52 | 100 |
| 8.1 | 10.92 | 19 |
| 10.7 | 8.30 | 13 |
| 13.6 | 6.49 | 8 |
| 14.1 | 6.27 | 9 |
| 14.7 | 6.04 | 7 |
| 15.3 | 5.78 | 12 |
| 17.7 | 5.00 | 9 |
| 18.5 | 4.79 | 8 |
| 20.4 | 4.36 | 8 |
| 21.2 | 4.19 | 11 |
| 21.7 | 4.10 | 5 |
| 22.1 | 4.02 | 10 |
| 23.2 | 3.83 | 37 |
| 24.0 | 3.71 | 13 |
| 24.6 | 3.62 | 10 |
| 25.3 | 3.52 | 30 |
| 26.0 | 3.43 | 10 |
| 26.3 | 3.39 | 13 |
| 26.6 | 3.35 | 12 |
| 27.2 | 3.28 | 5 |
| 27.5 | 3.24 | 5 |
| 28.2 | 3.16 | 5 |
| 29.0 | 3.08 | 13 |
| 31.1 | 2.88 | 5 |
| 31.6 | 2.83 | 6 |
| 32.4 | 2.77 | 5 |
| 34.6 | 2.59 | 5 |
| 35.6 | 2.52 | 6 |
| 36.0 | 2.49 | 5 |
| 37.1 | 2.42 | 5 |
| 38.9 | 2.32 | 6 |

In some embodiments, the characterization data of 2C-B malate (malate+peaks) are as provided in FIG. 19 and Table 30. The XRPD signals include the signals corresponding to 2C-B malate and other signals.

TABLE 30
XRPD Signal angle data of 2C-B Malate (malate +
peaks). The XRPD signals include the signals corresponding
to 2C-B malate and other signals.

| Position (°2θ) | d-value | Relative |
|---|---|---|
| 10.2 | 8.67 | 8 |
| 12.0 | 7.38 | 34 |
| 14.6 | 6.08 | 15 |
| 14.9 | 5.93 | 12 |
| 15.8 | 5.62 | 7 |
| 16.5 | 5.38 | 49 |
| 17.3 | 5.14 | 11 |
| 17.5 | 5.06 | 34 |
| 17.8 | 4.97 | 49 |
| 18.7 | 4.75 | 16 |
| 19.1 | 4.64 | 20 |
| 19.8 | 4.48 | 59 |
| 20.4 | 4.35 | 32 |
| 21.1 | 4.21 | 15 |
| 21.9 | 4.05 | 25 |
| 23.3 | 3.82 | 50 |
| 23.9 | 3.72 | 100 |
| 24.4 | 3.65 | 60 |
| 25.0 | 3.56 | 26 |
| 25.5 | 3.50 | 43 |
| 25.9 | 3.43 | 11 |
| 26.2 | 3.40 | 17 |
| 26.5 | 3.36 | 9 |
| 27.2 | 3.27 | 18 |
| 27.6 | 3.23 | 15 |
| 28.0 | 3.18 | 7 |
| 29.3 | 3.05 | 11 |
| 29.8 | 2.99 | 15 |
| 30.2 | 2.96 | 10 |
| 30.4 | 2.94 | 10 |
| 30.8 | 2.90 | 23 |
| 31.9 | 2.81 | 15 |
| 33.2 | 2.70 | 11 |
| 33.8 | 2.65 | 18 |
| 34.8 | 2.58 | 34 |
| 36.2 | 2.48 | 14 |
| 37.0 | 2.43 | 9 |
| 37.8 | 2.38 | 13 |
| 38.1 | 2.36 | 9 |
| 38.8 | 2.32 | 15 |
| 39.1 | 2.30 | 13 |

In some embodiments, the characterization data of 2C-B Maleate (Form 1) are as provided in FIG. 20 and Table 31.

TABLE 31
XRPD Signal angle data of 2C-B Maleate (Form 1)

| Position (°2θ) | d-value | Relative |
|---|---|---|
| 5.7 | 15.56 | 23 |
| 9.3 | 9.49 | 15 |
| 10.7 | 8.25 | 35 |
| 11.2 | 7.87 | 34 |
| 14.4 | 6.15 | 12 |
| 14.8 | 5.97 | 16 |
| 16.9 | 5.24 | 8 |
| 18.2 | 4.87 | 22 |
| 18.6 | 4.77 | 29 |
| 19.0 | 4.67 | 14 |
| 19.3 | 4.61 | 38 |
| 19.6 | 4.53 | 33 |
| 19.9 | 4.46 | 26 |
| 20.3 | 4.37 | 26 |
| 21.7 | 4.10 | 100 |
| 22.1 | 4.02 | 44 |
| 22.9 | 3.88 | 65 |
| 23.5 | 3.79 | 18 |
| 24.1 | 3.69 | 13 |
| 25.1 | 3.55 | 80 |
| 25.7 | 3.47 | 50 |
| 26.2 | 3.40 | 16 |
| 26.8 | 3.32 | 28 |
| 28.3 | 3.16 | 16 |
| 29.9 | 2.99 | 26 |
| 30.2 | 2.96 | 31 |
| 30.9 | 2.90 | 25 |
| 31.3 | 2.85 | 17 |
| 31.8 | 2.82 | 14 |
| 32.1 | 2.79 | 11 |
| 33.2 | 2.70 | 15 |
| 33.7 | 2.66 | 12 |
| 34.2 | 2.62 | 12 |
| 35.5 | 2.53 | 11 |
| 35.9 | 2.50 | 12 |
| 36.4 | 2.47 | 14 |
| 37.7 | 2.39 | 12 |
| 38.3 | 2.35 | 14 |
| 39.5 | 2.28 | 14 |

In some embodiments, the characterization data of 2C-B malonate are as provided in FIG. 21 and Table 32.

TABLE 32
XRPD Signal angle data of 2C-B Malonate

| Position (°2θ) | d-value | Relative |
|---|---|---|
| 5.1 | 17.33 | 24 |
| 9.9 | 8.92 | 24 |
| 10.2 | 8.67 | 42 |
| 12.3 | 7.21 | 44 |
| 15.3 | 5.78 | 13 |
| 16.0 | 5.54 | 49 |
| 18.0 | 4.93 | 11 |
| 19.0 | 4.68 | 7 |
| 19.8 | 4.47 | 10 |
| 20.5 | 4.34 | 11 |
| 21.2 | 4.19 | 11 |
| 21.7 | 4.09 | 41 |
| 22.4 | 3.97 | 19 |
| 23.2 | 3.84 | 100 |
| 23.7 | 3.75 | 20 |
| 24.6 | 3.62 | 15 |
| 25.1 | 3.55 | 25 |
| 25.4 | 3.50 | 33 |
| 25.7 | 3.47 | 30 |
| 26.5 | 3.36 | 54 |
| 27.0 | 3.30 | 14 |
| 27.6 | 3.23 | 12 |
| 27.8 | 3.20 | 8 |
| 28.2 | 3.16 | 8 |
| 28.7 | 3.11 | 12 |
| 29.1 | 3.07 | 17 |
| 29.9 | 2.98 | 20 |
| 30.4 | 2.94 | 11 |
| 30.9 | 2.89 | 7 |
| 31.7 | 2.83 | 16 |
| 32.4 | 2.77 | 9 |
| 32.7 | 2.74 | 20 |
| 32.9 | 2.72 | 11 |
| 33.7 | 2.66 | 12 |
| 34.2 | 2.62 | 11 |
| 34.8 | 2.58 | 9 |
| 35.7 | 2.52 | 7 |
| 36.1 | 2.49 | 9 |
| 36.7 | 2.45 | 8 |
| 37.2 | 2.41 | 9 |
| 37.7 | 2.39 | 8 |
| 38.2 | 2.36 | 10 |
| 38.4 | 2.34 | 9 |
| 38.9 | 2.31 | 8 |

In some embodiments, the characterization data of 2C-B mesylate are as provided in FIG. 22 and Table 33.

TABLE 33
XRPD Signal angle data of 2C-B Mesylate

| Position (°2θ) | d-value | Relative |
|---|---|---|
| 5.7 | 15.45 | 45 |
| 11.4 | 7.76 | 69 |
| 13.1 | 6.76 | 12 |
| 15.4 | 5.75 | 34 |
| 17.6 | 5.04 | 67 |
| 17.7 | 5.00 | 49 |
| 18.1 | 4.91 | 29 |
| 19.1 | 4.66 | 84 |
| 19.2 | 4.62 | 41 |
| 19.7 | 4.52 | 23 |
| 20.1 | 4.42 | 51 |
| 20.4 | 4.35 | 31 |
| 21.3 | 4.18 | 60 |
| 22.5 | 3.96 | 85 |
| 22.7 | 3.92 | 57 |
| 23.8 | 3.73 | 41 |
| 24.2 | 3.68 | 19 |
| 24.9 | 3.58 | 15 |
| 25.6 | 3.48 | 77 |
| 25.7 | 3.46 | 67 |
| 25.9 | 3.44 | 100 |
| 26.2 | 3.40 | 20 |
| 27.2 | 3.28 | 15 |
| 28.0 | 3.18 | 23 |
| 28.2 | 3.16 | 36 |
| 28.7 | 3.11 | 26 |
| 30.1 | 2.97 | 18 |
| 30.5 | 2.93 | 11 |
| 30.8 | 2.90 | 14 |
| 31.1 | 2.88 | 16 |
| 33.7 | 2.66 | 17 |
| 33.9 | 2.64 | 25 |
| 34.3 | 2.62 | 12 |
| 34.8 | 2.58 | 16 |
| 35.6 | 2.52 | 17 |
| 38.3 | 2.35 | 12 |
| 39.1 | 2.30 | 17 |

In some embodiments, the characterization data of 2C-B Mucate are as provided in FIG. 23 and Table 34.

TABLE 34
XRPD Signal angle data of 2C-B Mucate

| Position (°2θ) | d-value | Relative |
|---|---|---|
| 9.9 | 8.97 | 55 |
| 12.0 | 7.36 | 33 |
| 14.8 | 5.97 | 19 |
| 15.5 | 5.72 | 100 |
| 18.1 | 4.91 | 9 |
| 19.6 | 4.53 | 43 |
| 20.2 | 4.40 | 45 |
| 21.2 | 4.19 | 13 |
| 22.1 | 4.03 | 40 |
| 22.4 | 3.96 | 14 |
| 23.8 | 3.74 | 12 |
| 24.2 | 3.68 | 40 |
| 24.9 | 3.58 | 84 |
| 26.8 | 3.33 | 10 |
| 27.1 | 3.29 | 18 |
| 27.5 | 3.24 | 19 |
| 28.3 | 3.15 | 16 |
| 28.6 | 3.12 | 16 |
| 28.9 | 3.09 | 16 |
| 29.4 | 3.04 | 14 |
| 30.6 | 2.92 | 24 |
| 32.3 | 2.77 | 22 |
| 34.4 | 2.61 | 23 |
| 34.7 | 2.58 | 14 |
| 35.2 | 2.55 | 14 |
| 35.5 | 2.53 | 13 |
| 36.6 | 2.45 | 16 |
| 38.7 | 2.32 | 10 |
| 39.9 | 2.26 | 12 |

In some embodiments, the characterization data of 2C-B succinate (Form 1 and Form 2 plus free succinic acid) are as provided in FIG. 24 and Table 35.

TABLE 35
XRPD Signal angle data of 2C-B Succinate (Form
1 and Form 2 plus free succinic acid)

| Position (°2θ) | d-value | Relative |
|---|---|---|
| 7.5 | 11.76 | 27 |
| 8.2 | 10.78 | 28 |
| 9.1 | 9.72 | 23 |
| 10.6 | 8.33 | 14 |
| 11.1 | 7.94 | 27 |
| 12.5 | 7.07 | 28 |
| 12.7 | 6.98 | 19 |
| 12.9 | 6.85 | 22 |
| 13.1 | 6.76 | 26 |
| 13.4 | 6.63 | 13 |
| 13.9 | 6.35 | 14 |
| 14.2 | 6.22 | 31 |
| 14.8 | 5.98 | 14 |
| 15.8 | 5.60 | 12 |
| 16.4 | 5.41 | 61 |
| 16.7 | 5.30 | 89 |
| 17.6 | 5.04 | 100 |
| 18.2 | 4.87 | 26 |
| 18.9 | 4.69 | 29 |
| 19.5 | 4.54 | 17 |
| 19.8 | 4.48 | 15 |
| 20.7 | 4.30 | 31 |
| 21.0 | 4.22 | 28 |
| 21.3 | 4.17 | 58 |
| 21.8 | 4.08 | 61 |
| 21.8 | 4.08 | 61 |
| 22.0 | 4.04 | 33 |
| 22.3 | 3.98 | 23 |
| 22.5 | 3.96 | 34 |
| 22.6 | 3.93 | 50 |
| 22.8 | 3.89 | 33 |
| 23.6 | 3.77 | 37 |
| 23.9 | 3.73 | 35 |
| 24.3 | 3.66 | 50 |
| 24.6 | 3.61 | 72 |
| 25.1 | 3.55 | 91 |
| 25.2 | 3.53 | 48 |
| 25.6 | 3.47 | 63 |
| 25.9 | 3.43 | 96 |
| 26.2 | 3.41 | 39 |
| 26.8 | 3.32 | 25 |
| 27.1 | 3.29 | 19 |
| 27.3 | 3.26 | 18 |
| 27.5 | 3.25 | 20 |
| 28.0 | 3.18 | 40 |
| 28.4 | 3.15 | 19 |
| 28.8 | 3.10 | 17 |
| 29.4 | 3.04 | 29 |
| 29.9 | 2.99 | 25 |
| 30.1 | 2.97 | 23 |
| 30.3 | 2.95 | 22 |
| 31.3 | 2.85 | 24 |
| 31.7 | 2.82 | 17 |
| 32.1 | 2.79 | 16 |
| 32.6 | 2.75 | 14 |
| 33.4 | 2.69 | 20 |
| 33.9 | 2.64 | 25 |
| 34.9 | 2.57 | 21 |
| 35.4 | 2.53 | 18 |
| 36.2 | 2.48 | 14 |
| 36.6 | 2.46 | 15 |
| 38.0 | 2.37 | 21 |
| 38.7 | 2.33 | 15 |
| 39.7 | 2.27 | 18 |

In some embodiments, the characterization data of 2C-B tartrate (Form 1) are as provided in FIG. 25 and Table 36.

TABLE 36
XRPD Signal angle data of 2C-B Tartrate (Form 1)

| Position (°2θ) | d-value | Relative |
|---|---|---|
| 10.2 | 8.66 | 37 |
| 12.8 | 6.89 | 26 |
| 14.2 | 6.24 | 24 |
| 14.3 | 6.19 | 40 |
| 15.4 | 5.75 | 18 |
| 16.6 | 5.35 | 16 |
| 17.4 | 5.11 | 19 |
| 18.1 | 4.91 | 9 |
| 20.2 | 4.40 | 88 |
| 20.5 | 4.33 | 51 |
| 21.6 | 4.11 | 16 |
| 23.2 | 3.84 | 51 |
| 23.9 | 3.72 | 27 |
| 24.2 | 3.68 | 31 |
| 24.7 | 3.61 | 100 |
| 25.6 | 3.49 | 17 |
| 26.0 | 3.42 | 15 |
| 26.6 | 3.35 | 8 |
| 27.2 | 3.28 | 46 |
| 27.5 | 3.25 | 14 |
| 28.4 | 3.15 | 6 |
| 28.6 | 3.12 | 9 |
| 28.9 | 3.09 | 12 |
| 29.6 | 3.02 | 12 |
| 30.6 | 2.92 | 5 |
| 31.0 | 2.89 | 5 |
| 31.5 | 2.84 | 10 |
| 31.7 | 2.82 | 23 |
| 32.1 | 2.79 | 6 |
| 32.5 | 2.75 | 9 |
| 32.8 | 2.73 | 6 |
| 33.2 | 2.70 | 7 |
| 33.7 | 2.66 | 14 |
| 34.2 | 2.62 | 20 |
| 34.7 | 2.59 | 10 |
| 35.1 | 2.56 | 7 |
| 35.9 | 2.50 | 11 |
| 36.5 | 2.46 | 6 |
| 37.4 | 2.41 | 10 |
| 38.0 | 2.37 | 9 |
| 38.4 | 2.34 | 7 |
| 39.0 | 2.31 | 12 |
| 39.2 | 2.30 | 9 |
| 39.6 | 2.28 | 9 |

In some embodiments, the characterization data of 2C-B Tosylate (Form 1) are as provided in FIG. 26 and Table 37.

TABLE 37
XRPD Signal angle data of 2C-B Tosylate (Form 1)

| Position (°2θ) | d-value | Relative |
|---|---|---|
| 6.2 | 14.21 | 29 |
| 11.6 | 7.63 | 18 |
| 12.9 | 6.84 | 14 |
| 16.4 | 5.40 | 40 |
| 16.8 | 5.29 | 21 |
| 18.3 | 4.86 | 53 |
| 20.7 | 4.29 | 100 |
| 22.3 | 3.98 | 34 |
| 23.3 | 3.82 | 54 |
| 24.0 | 3.70 | 14 |
| 24.2 | 3.67 | 19 |
| 25.2 | 3.54 | 14 |
| 25.9 | 3.45 | 19 |
| 26.6 | 3.35 | 16 |
| 28.5 | 3.13 | 29 |
| 29.2 | 3.05 | 15 |
| 31.5 | 2.84 | 13 |
| 31.9 | 2.81 | 11 |
| 33.3 | 2.69 | 11 |
| 33.9 | 2.65 | 12 |
| 35.2 | 2.55 | 19 |
| 35.7 | 2.52 | 10 |
| 37.0 | 2.43 | 14 |
| 37.3 | 2.41 | 12 |
| 38.4 | 2.35 | 19 |

In some embodiments, the 1H NMR spectrum of 2C-B (free base) is as provided in FIG. 27.

In some embodiments, the characterization data of a sample comprising crystalline 2C-B (free base Form 1) are as provided in FIG. 6 and Table 11.

In some embodiments, the characterization data of 2C-B B.esylate (Form 2) are as provided in FIG. 29 and Table 38.

TABLE 38
XRPD Signal angle data of 2C-B Besylate (Form 2)

| Position (°2θ) | d-value | Relative |
|---|---|---|
| 5.7 | 15.45 | 10 |
| 9.3 | 9.47 | 4 |
| 10.4 | 8.49 | 4 |
| 11.4 | 7.73 | 30 |
| 14.0 | 6.34 | 5 |
| 14.9 | 5.96 | 6 |
| 15.5 | 5.72 | 11 |
| 16.4 | 5.42 | 38 |
| 17.0 | 5.23 | 17 |
| 17.1 | 5.19 | 23 |
| 17.7 | 5.02 | 8 |
| 18.1 | 4.90 | 6 |
| 18.6 | 4.77 | 18 |
| 19.0 | 4.68 | 54 |
| 20.0 | 4.43 | 100 |
| 20.4 | 4.36 | 16 |
| 20.8 | 4.27 | 17 |
| 21.7 | 4.10 | 20 |
| 22.0 | 4.04 | 11 |
| 22.9 | 3.89 | 34 |
| 24.1 | 3.70 | 45 |
| 24.9 | 3.57 | 37 |
| 25.6 | 3.48 | 30 |
| 26.0 | 3.42 | 14 |
| 26.4 | 3.37 | 22 |
| 26.7 | 3.34 | 11 |
| 27.7 | 3.22 | 7 |
| 28.1 | 3.17 | 16 |
| 29.3 | 3.05 | 14 |
| 29.8 | 3.00 | 15 |
| 30.0 | 2.98 | 12 |
| 30.4 | 2.94 | 7 |
| 31.1 | 2.87 | 17 |
| 31.4 | 2.85 | 13 |
| 32.2 | 2.78 | 8 |
| 32.7 | 2.74 | 7 |
| 33.6 | 2.67 | 7 |
| 34.2 | 2.62 | 8 |
| 34.8 | 2.58 | 12 |
| 35.4 | 2.54 | 12 |
| 35.8 | 2.51 | 12 |
| 36.1 | 2.49 | 8 |
| 36.4 | 2.47 | 7 |
| 37.0 | 2.43 | 6 |
| 37.5 | 2.40 | 8 |
| 37.8 | 2.38 | 8 |
| 38.4 | 2.34 | 7 |
| 39.4 | 2.29 | 5 |

In some embodiments, the characterization data of 2C-B Esylate (Form 2) are as provided in FIG. 30 and Table 39.

TABLE 39
XRPD Signal angle data of 2C-B Esylate (Form 2)

| Position (°2θ) | d-value | Relative |
|---|---|---|
| 6.3 | 14.07 | 9 |
| 8.6 | 10.23 | 29 |
| 11.9 | 7.44 | 8 |
| 12.5 | 7.06 | 29 |
| 13.3 | 6.66 | 11 |
| 13.9 | 6.38 | 67 |
| 14.2 | 6.24 | 9 |
| 15.2 | 5.83 | 49 |
| 16.8 | 5.26 | 16 |
| 17.5 | 5.07 | 41 |
| 17.9 | 4.94 | 9 |
| 18.0 | 4.93 | 9 |
| 18.0 | 4.92 | 9 |
| 18.1 | 4.90 | 10 |
| 19.7 | 4.50 | 14 |
| 20.3 | 4.37 | 7 |
| 21.5 | 4.13 | 13 |
| 21.9 | 4.06 | 13 |
| 22.3 | 3.98 | 8 |
| 22.7 | 3.91 | 62 |
| 23.9 | 3.73 | 39 |
| 24.1 | 3.70 | 100 |
| 24.3 | 3.66 | 39 |
| 24.6 | 3.62 | 11 |
| 24.8 | 3.59 | 17 |
| 25.2 | 3.54 | 13 |
| 25.3 | 3.51 | 7 |
| 25.9 | 3.44 | 29 |
| 27.0 | 3.30 | 30 |
| 27.5 | 3.25 | 24 |
| 27.9 | 3.20 | 10 |
| 28.1 | 3.18 | 8 |
| 29.0 | 3.08 | 17 |
| 29.5 | 3.03 | 19 |
| 29.6 | 3.02 | 16 |
| 29.9 | 2.98 | 7 |
| 30.6 | 2.93 | 12 |
| 31.0 | 2.88 | 7 |
| 31.1 | 2.88 | 6 |
| 31.6 | 2.83 | 14 |
| 32.2 | 2.78 | 12 |
| 32.3 | 2.77 | 11 |
| 32.4 | 2.76 | 12 |
| 33.1 | 2.70 | 8 |
| 33.2 | 2.70 | 9 |
| 33.2 | 2.70 | 8 |
| 34.1 | 2.63 | 12 |
| 34.2 | 2.62 | 10 |
| 35.0 | 2.56 | 13 |
| 35.5 | 2.53 | 6 |
| 36.7 | 2.45 | 10 |
| 36.8 | 2.44 | 7 |
| 37.9 | 2.37 | 9 |
| 39.4 | 2.29 | 8 |
| 39.5 | 2.28 | 8 |
| 40.0 | 2.26 | 15 |

In some embodiments, the characterization data of 2C-B Phosphate (Form 2) are as provided in FIG. 31 and Table 40.

TABLE 40
XRPD Signal angle data of 2C-B Phosphate (Form 2)

| Position (°2θ) | d-value | Relative |
|---|---|---|
| 8.6 | 10.31 | 62 |
| 12.9 | 6.87 | 51 |
| 14.9 | 5.95 | 12 |
| 16.3 | 5.45 | 16 |
| 17.2 | 5.17 | 13 |
| 17.7 | 5.01 | 21 |
| 19.4 | 4.58 | 12 |
| 21.1 | 4.21 | 22 |
| 21.5 | 4.13 | 59 |
| 22.9 | 3.88 | 29 |
| 24.8 | 3.59 | 100 |
| 25.9 | 3.44 | 50 |
| 28.7 | 3.11 | 58 |
| 32.8 | 2.73 | 31 |
| 34.8 | 2.58 | 27 |
| 37.0 | 2.43 | 17 |
| 37.7 | 2.39 | 14 |
| 38.3 | 2.35 | 12 |
| 39.3 | 2.29 | 33 |

In some embodiments, the characterization data of 2C-B Lactate (Form 2) are as provided in FIG. 32 and Table 41.

TABLE 41
XRPD Signal angle data of 2C-B Lactate (Form 2)

| Position (°2θ) | d-value | Relative |
|---|---|---|
| 5.3 | 16.67 | 74 |
| 7.4 | 11.95 | 37 |
| 8.4 | 10.48 | 17 |
| 10.5 | 8.41 | 88 |
| 11.0 | 8.01 | 9 |
| 11.3 | 7.86 | 12 |
| 12.4 | 7.12 | 9 |
| 13.4 | 6.62 | 7 |
| 14.7 | 6.01 | 13 |
| 15.5 | 5.73 | 40 |
| 16.3 | 5.44 | 74 |
| 17.1 | 5.18 | 18 |
| 17.7 | 5.00 | 10 |
| 19.4 | 4.57 | 100 |
| 20.6 | 4.31 | 14 |
| 21.9 | 4.06 | 8 |
| 22.7 | 3.92 | 32 |
| 23.1 | 3.84 | 55 |
| 23.4 | 3.80 | 22 |
| 23.6 | 3.76 | 31 |
| 24.3 | 3.67 | 13 |
| 24.5 | 3.64 | 18 |
| 25.0 | 3.56 | 34 |
| 25.2 | 3.53 | 100 |
| 25.9 | 3.43 | 14 |
| 26.4 | 3.38 | 47 |
| 26.7 | 3.34 | 28 |
| 27.5 | 3.24 | 15 |
| 27.8 | 3.21 | 25 |
| 27.9 | 3.20 | 32 |
| 28.8 | 3.10 | 25 |
| 29.9 | 2.99 | 10 |
| 30.5 | 2.93 | 9 |
| 30.8 | 2.91 | 10 |
| 31.1 | 2.87 | 37 |
| 31.4 | 2.85 | 21 |
| 31.7 | 2.82 | 10 |
| 32.4 | 2.76 | 10 |
| 32.7 | 2.74 | 13 |
| 33.3 | 2.69 | 9 |
| 34.1 | 2.63 | 12 |
| 34.5 | 2.60 | 15 |
| 35.0 | 2.56 | 15 |
| 35.7 | 2.52 | 14 |
| 36.2 | 2.48 | 11 |
| 36.6 | 2.46 | 15 |
| 37.2 | 2.42 | 19 |
| 37.7 | 2.39 | 10 |
| 38.9 | 2.32 | 17 |
| 39.4 | 2.29 | 10 |

In some embodiments, the characterization data of 2C-B Maleate (Form 2) are as provided in FIG. 33 and Table 42.

TABLE 42
XRPD Signal angle data of 2C-B Maleate (Form 2)

| Position (°2θ) | d-value | Relative |
|---|---|---|
| 7.0 | 12.59 | 7 |
| 10.1 | 8.74 | 20 |
| 10.8 | 8.18 | 5 |
| 12.9 | 6.86 | 5 |
| 14.0 | 6.31 | 10 |
| 15.2 | 5.83 | 24 |
| 16.5 | 5.37 | 6 |
| 17.1 | 5.19 | 7 |
| 18.2 | 4.87 | 25 |
| 19.3 | 4.61 | 9 |
| 19.9 | 4.47 | 18 |
| 20.2 | 4.39 | 6 |
| 21.0 | 4.22 | 11 |
| 23.0 | 3.86 | 8 |
| 23.8 | 3.74 | 26 |
| 24.3 | 3.66 | 14 |
| 24.9 | 3.58 | 8 |
| 25.6 | 3.49 | 33 |
| 25.9 | 3.44 | 100 |
| 26.2 | 3.40 | 38 |
| 27.4 | 3.25 | 11 |
| 27.8 | 3.21 | 22 |
| 28.1 | 3.18 | 16 |
| 29.2 | 3.06 | 18 |
| 29.6 | 3.02 | 12 |
| 30.0 | 2.98 | 10 |
| 30.5 | 2.93 | 15 |
| 32.2 | 2.78 | 10 |
| 34.9 | 2.57 | 8 |
| 35.5 | 2.53 | 8 |
| 36.4 | 2.47 | 6 |
| 36.8 | 2.44 | 8 |
| 38.2 | 2.36 | 6 |
| 39.3 | 2.29 | 7 |
| 39.9 | 2.26 | 8 |

In some embodiments, the characterization data of 2C-B Succinate (Form 1) are as provided in FIG. 34 and Table 43.

TABLE 43
XRPD Signal angle data of 2C-B Succinate (Form 1)

| Position (°2θ) | d-value | Relative |
|---|---|---|
| 7.6 | 11.69 | 28 |
| 12.8 | 6.91 | 9 |
| 13.0 | 6.83 | 16 |
| 13.1 | 6.75 | 18 |
| 13.4 | 6.62 | 7 |
| 14.0 | 6.33 | 8 |
| 16.8 | 5.26 | 42 |
| 17.6 | 5.03 | 87 |
| 18.4 | 4.83 | 13 |
| 19.2 | 4.61 | 5 |
| 21.3 | 4.17 | 26 |
| 21.5 | 4.13 | 13 |
| 21.9 | 4.07 | 24 |
| 22.1 | 4.03 | 16 |
| 22.7 | 3.92 | 100 |
| 23.0 | 3.87 | 11 |
| 23.9 | 3.72 | 36 |
| 25.1 | 3.55 | 29 |
| 25.7 | 3.46 | 19 |
| 26.0 | 3.43 | 93 |
| 26.3 | 3.38 | 12 |
| 26.7 | 3.34 | 8 |
| 26.9 | 3.32 | 17 |
| 27.8 | 3.20 | 13 |
| 28.2 | 3.17 | 24 |
| 28.4 | 3.14 | 16 |
| 29.9 | 2.99 | 12 |
| 30.2 | 2.96 | 13 |
| 30.4 | 2.94 | 28 |
| 31.2 | 2.87 | 4 |
| 31.4 | 2.85 | 8 |
| 32.3 | 2.77 | 11 |
| 33.4 | 2.68 | 14 |
| 34.0 | 2.64 | 13 |
| 35.0 | 2.57 | 4 |
| 35.5 | 2.53 | 15 |
| 36.6 | 2.46 | 13 |
| 37.1 | 2.42 | 8 |
| 37.5 | 2.40 | 9 |
| 38.2 | 2.36 | 8 |
| 38.8 | 2.32 | 8 |
| 39.2 | 2.30 | 8 |
| 39.8 | 2.27 | 18 |

In some embodiments, the characterization data of 2C-B tosylate (Forms 1 and 2) are as provided in FIG. 35 and Table 44.

TABLE 44
XRPD Signal angle data of 2C-B Tosylate (Forms 1 and 2)

| Position (°2θ) | d-value | Relative | |
|---|---|---|---|
| 2.8 | 31.11 | 11 | |
| 5.6 | 15.78 | 18 | |
| 8.0 | 11.02 | 17 | Tosylate (Form 2) |
| 11.6 | 7.62 | 14 | |
| 12.6 | 7.03 | 37 | Tosylate (Form 2) |
| 13.0 | 6.81 | 30 | Tosylate (Form 2) |
| 13.6 | 6.49 | 17 | |
| 14.6 | 6.06 | 15 | |
| 16.4 | 5.39 | 82 | |
| 16.8 | 5.28 | 42 | |
| 18.0 | 4.93 | 37 | |
| 18.3 | 4.85 | 33 | |
| 19.2 | 4.63 | 22 | Tosylate (Form 2) |
| 20.7 | 4.28 | 78 | Tosylate (Form 2) |
| 21.0 | 4.23 | 23 | |
| 21.4 | 4.15 | 20 | |
| 22.5 | 3.96 | 37 | |
| 22.7 | 3.92 | 35 | |
| 23.3 | 3.82 | 100 | |
| 24.1 | 3.70 | 37 | |
| 24.8 | 3.59 | 45 | |
| 25.3 | 3.52 | 47 | |
| 25.9 | 3.44 | 33 | |
| 26.1 | 3.42 | 27 | |
| 26.5 | 3.37 | 23 | |
| 27.1 | 3.29 | 49 | |
| 27.7 | 3.22 | 26 | |
| 28.1 | 3.17 | 22 | |
| 28.5 | 3.13 | 43 | |
| 29.3 | 3.05 | 33 | |
| 29.9 | 2.98 | 23 | |
| 30.4 | 2.94 | 30 | |
| 31.6 | 2.83 | 15 | |
| 31.9 | 2.81 | 15 | |
| 32.3 | 2.77 | 17 | |
| 33.3 | 2.69 | 22 | |
| 33.9 | 2.64 | 22 | |
| 35.2 | 2.55 | 20 | |
| 35.7 | 2.52 | 15 | |
| 37.0 | 2.43 | 18 | |
| 37.8 | 2.38 | 18 | |
| 38.4 | 2.35 | 33 | |
| 39.1 | 2.30 | 19 | |
| 39.8 | 2.26 | 21 | |

In some embodiments, the characterization data of a sample comprising 2C-B free base (Form 1 and Form 2) are as provided in FIG. 5 and Table 10.

In some embodiments, the characterization data of 2C-B Aspartate (aspartate+peaks) are as provided in FIG. 37 and Table 45. The XRPD signals include the signals corresponding to 2C-B aspartate and other signals.

TABLE 45
XRPD Signal angle data of 2C-B Aspartate (aspartate +
peaks). The XRPD signals include the signals corresponding
to 2C-B aspartate and other signals.

| Position (°2θ) | d-value | Relative | Assignment |
|---|---|---|---|
| 4.1 | 21.55 | 51 | aspartate |
| 8.2 | 10.81 | 64 | aspartate |
| 11.7 | 7.54 | 20 | Extra peak |
| 12.3 | 7.18 | 23 | aspartate |
| 13.7 | 6.46 | 46 | aspartate |
| 15.0 | 5.91 | 18 | aspartate |
| 16.5 | 5.38 | 96 | aspartate |
| 17.5 | 5.06 | 17 | aspartate |
| 18.4 | 4.81 | 37 | Extra peak |
| 19.0 | 4.67 | 33 | aspartate |
| 19.8 | 4.49 | 36 | Extra peak |
| 20.5 | 4.32 | 32 | Extra peak |
| 21.6 | 4.12 | 30 | aspartate |
| 22.0 | 4.04 | 71 | aspartate |
| 22.4 | 3.97 | 78 | Extra peak |
| 23.5 | 3.79 | 56 | aspartate |
| 23.7 | 3.75 | 67 | aspartate |
| 24.2 | 3.67 | 100 | aspartate |
| 25.1 | 3.55 | 30 | aspartate |
| 27.1 | 3.29 | 82 | aspartate |
| 27.5 | 3.24 | 25 | aspartate |
| 29.1 | 3.07 | 36 | aspartate |
| 29.7 | 3.00 | 26 | aspartate |
| 30.4 | 2.94 | 27 | aspartate |
| 31.0 | 2.88 | 42 | Extra peak |
| 31.7 | 2.82 | 23 | aspartate |
| 33.3 | 2.69 | 19 | aspartate |
| 33.8 | 2.65 | 18 | aspartate |
| 35.0 | 2.57 | 24 | aspartate |
| 37.2 | 2.42 | 26 | Extra peak |
| 38.3 | 2.35 | 28 | aspartate |
| 39.2 | 2.30 | 23 | aspartate |

In some embodiments, the characterization data of 2C-B Glutamate (PO) are as provided in FIG. 38 and Table 46.

TABLE 46
XRPD Signal angle data of 2C-B Glutamate (PO)

| Position (°2θ) | d-value | Relative |
|---|---|---|
| 3.7 | 24.14 | 100 |
| 7.3 | 12.14 | 74 |
| 14.5 | 6.09 | 44 |
| 16.4 | 5.42 | 18 |
| 16.6 | 5.35 | 6 |
| 16.9 | 5.24 | 7 |
| 17.0 | 5.21 | 6 |
| 18.0 | 4.93 | 5 |
| 18.2 | 4.88 | 6 |
| 19.0 | 4.67 | 6 |
| 20.1 | 4.43 | 5 |
| 21.9 | 4.05 | 69 |
| 23.2 | 3.84 | 5 |
| 23.5 | 3.78 | 13 |
| 24.4 | 3.64 | 12 |
| 25.1 | 3.55 | 10 |
| 25.6 | 3.49 | 36 |
| 27.4 | 3.26 | 7 |
| 27.9 | 3.20 | 9 |
| 28.4 | 3.14 | 28 |
| 29.3 | 3.05 | 24 |
| 30.0 | 2.98 | 5 |
| 30.5 | 2.93 | 8 |
| 31.8 | 2.81 | 30 |
| 32.8 | 2.73 | 6 |
| 35.3 | 2.54 | 9 |
| 35.8 | 2.51 | 10 |
| 36.8 | 2.44 | 10 |
| 38.9 | 2.31 | 12 |
| 39.6 | 2.28 | 5 |

In some embodiments, the characterization data of 2C-B Citrate (Form 2) are as provided in FIG. 39 and Table 47.

TABLE 47
XRPD Signal angle data of 2C-B Citrate (Form 2)

| Position (°2θ) | d-value | Relative |
|---|---|---|
| 3.7 | 24.14 | 22 |
| 7.3 | 12.18 | 9 |
| 10.3 | 8.59 | 16 |
| 11.5 | 7.68 | 5 |
| 12.1 | 7.34 | 15 |
| 12.5 | 7.09 | 11 |
| 12.7 | 6.97 | 9 |
| 13.1 | 6.77 | 26 |
| 13.6 | 6.51 | 19 |
| 14.0 | 6.33 | 4 |
| 14.5 | 6.11 | 41 |
| 15.5 | 5.72 | 11 |
| 16.3 | 5.45 | 17 |
| 16.6 | 5.33 | 62 |
| 17.4 | 5.09 | 7 |
| 17.7 | 5.01 | 8 |
| 17.9 | 4.96 | 5 |
| 18.1 | 4.89 | 4 |
| 18.5 | 4.79 | 5 |
| 19.2 | 4.61 | 15 |
| 19.5 | 4.55 | 8 |
| 20.2 | 4.39 | 12 |
| 20.6 | 4.30 | 100 |
| 21.3 | 4.18 | 14 |
| 21.8 | 4.08 | 94 |
| 22.1 | 4.02 | 13 |
| 22.5 | 3.96 | 20 |
| 23.1 | 3.85 | 19 |
| 23.3 | 3.82 | 16 |
| 23.6 | 3.76 | 34 |
| 24.1 | 3.70 | 20 |
| 24.4 | 3.65 | 59 |
| 24.9 | 3.58 | 9 |
| 25.2 | 3.53 | 8 |
| 25.5 | 3.50 | 11 |
| 25.8 | 3.46 | 24 |
| 26.3 | 3.39 | 51 |
| 26.6 | 3.36 | 13 |
| 26.8 | 3.32 | 59 |
| 27.2 | 3.27 | 21 |
| 27.9 | 3.20 | 10 |
| 28.2 | 3.16 | 22 |
| 28.8 | 3.10 | 9 |
| 29.2 | 3.06 | 13 |
| 29.9 | 2.98 | 9 |
| 30.2 | 2.96 | 35 |
| 30.2 | 2.96 | 30 |
| 30.7 | 2.91 | 8 |
| 31.2 | 2.87 | 7 |
| 31.9 | 2.81 | 9 |
| 32.8 | 2.73 | 16 |
| 33.4 | 2.68 | 12 |
| 33.5 | 2.67 | 12 |
| 33.9 | 2.64 | 8 |
| 34.6 | 2.59 | 7 |
| 34.9 | 2.57 | 7 |
| 35.1 | 2.55 | 8 |
| 35.8 | 2.51 | 6 |
| 36.4 | 2.47 | 14 |
| 36.7 | 2.45 | 10 |
| 36.8 | 2.44 | 10 |
| 36.9 | 2.44 | 9 |
| 37.7 | 2.39 | 6 |
| 38.2 | 2.36 | 8 |
| 39.0 | 2.31 | 14 |
| 39.0 | 2.31 | 12 |
| 39.9 | 2.26 | 8 |

Instrumental Techniques

The data summarized above were gathered as follows: For X-ray Powder Diffraction (XRPD), a Rigaku Smart-Lab X-ray diffraction system was configured for reflection Bragg-Brentano geometry using a line source X-ray beam. The X-ray source is a Cu Long Fine Focus tube that was operated at 40 kV and 44 ma. That source provides an incident beam profile at the sample that changes from a narrow line at high angles to a broad rectangle at low angles. Beam conditioning slits are used on the line X-ray source to ensure that the maximum beam size is less than 10 mm both along the line and normal to the line. The Bragg-Brentano geometry is a para-focusing geometry controlled by passive divergence and receiving slits with the sample itself acting as the focusing component for the optics. The inherent resolution of Bragg-Brentano geometry is governed in part by the diffractometer radius and the width of the receiving slit used. Typically, the Rigaku Smart-Lab is operated to give peak widths of 0.1 °2θ or less. The axial divergence of the X-ray beam is controlled by 5.0-degree Soller slits in both the incident and diffracted beam paths.

Powder samples were prepared in a low background Si holder using light manual pressure to keep the sample surfaces flat and level with the reference surface of the sample holder. Each sample was analyzed from 2 to 40 °2θ using a continuous scan of 6 °2θ per minute with an effective step size of 0.02 °2θ.

Nuclear Magnetic Resonance (1H NMR) spectra were acquired on a Bruker Avance II 400 spectrometer. Samples were prepared by dissolving material in a suitable solvent, such as a deuterated solvent, such as CDCl₃ or DMSO-d6. The solutions were filtered and placed into individual 5-mm NMR tubes for subsequent spectral acquisition. The temperature controlled (295K) 1H NMR spectra acquired on the Avance II 400 utilized a 5-mm cryoprobe operating at an observing frequency of 400.18 MHz.

### Example 3—Polymorph Screen

The active pharmaceutical ingredient (API), which may be a free base or a salt, is characterized to evaluate its physical properties. The evaluation is performed by X-ray powder diffraction (XRPD), polarized light microscopy (PLM), differential scanning calorimetry (DSC), thermogravimetry (TG), dynamic vapor sorption/desorption (DVS), and/or solubility testing in organic solvents, water, and mixed solvent systems. XRPD data is used to assess crystallinity. PLM data is used to evaluate crystallinity and particle size/morphology. DSC data is used to evaluate melting point, thermal stability, and crystalline form conversion. TG data is used to evaluate if the API is a solvate or hydrate, and to evaluate thermal stability. DVS data is used to evaluate hygroscopicity of the API and if hydrates can be formed at high relative humidity. About 10 to 15 solvents may be selected from the list below, based on their properties (polarity, dielectric constant and dipole moment).

TABLE 48
Solvents

| | |
|---|---|
| acetic acid | n-heptane |
| acetone | n-hexane |
| acetonitrile | 1,1,1,3,3,3-hexafluoro-2-propanol |
| benzyl alcohol | isobutanol (2-methyl-1-propanol) |
| 1-butanol | isopentanol (3-methyl-1-butanol) |
| 2-butanol | isopropyl alcohol (2-propanol) |
| butyl acetate | isopropylbenzene (cumene) |
| t-butyl methyl ether | methanol |
| chlorobenzene | methoxybenzene (anisole) |
| chloroform | methyl acetate |
| di(ethylene glycol) | methyl ethyl ketone (2-butanone ) |
| dichloromethane | methyl isobutyl ketone |
| diethyl ether | nitromethane |
| diethylamine | N-methyl-2-pyrrolidone (NMP) |
| Dimethylacetamide (DMA) | 1-octanol |
| diisopropyl ether | 1-pentanol |
| N,N-dimethyl-formamide (DMF) | 1-propanol |
| dimethyl sulfoxide | perfluorohexane |
| 1,4-dioxane | propyl acetate |
| 1,2-ethanediol (ethylene glycol) | 1,1,2,2-tetrachloroethane |
| ethanol | tetrahydrofuran |
| ethanolamine | toluene |
| 2-ethoxyethanol (Cellusolve) | 1,1,1-trichloroethane |
| ethyl acetate | 2,2,2-trifluoroethanol |
| ethyl formate | water |
| formic acid | o-xylene (1,2-dimethylbenzene) |
| glycerol | p-xylene (1,4-dimethylbenzene) |

The information obtained is used for designing the subsequent polymorph screen. Solvents are used as a single solvent or as solvent mixtures, some containing water. The techniques used for the polymorph screen are chosen based on the solvent selected and properties of the API. The following techniques (or a combination of techniques) may be used for the polymorph screening;
API is dissolved in a solvent or mixture of solvents, and the solvents are evaporated at different rates (slow evaporation or fast evaporation) and at different temperatures (ambient or elevated).
API is dissolved in a solvent or mixture of solvents (at ambient temperature or an elevated temperature), and the final solution is cooled (between −78° C. to 20° C.). The cooling method can be a fast cooling (by plunging the sample to an ice bath or a dry ice/acetone bath), or slow cooling. The solids formed will be recovered by filtration and dried (air dried or vacuum dried).
API is dissolved in a solvent or mixture of solvents, and an antisolvent is added to precipitate the salt. The solids formed will be recovered by filtration and dried (air dried or vacuum dried).
API is added to a solvent or mixture of solvents, where the API is not fully dissolved. The slurry will be agitated at different temperatures for a number of days. The solids formed will be recovered by filtration and (air dried or vacuum dried).
API is milled (by mechanical milling or by mortar and pestle), with a drop of solvent, or without any solvent.
API is melted and cooled (at different cooling rates, fast and slow, and cooled to different temperatures) to obtain solids.
API is suspended in a solvent or mixture of solvents, and the slurry is placed in a heating/cooling cycle for multiple cycles. The remaining solids after the final cooling cycle will be filtered and (air dried or vacuum dried).
API is processed to obtain an amorphous form (by melting, milling, solvent evaporation, spray drying or lyophilization). The amorphous form will then be exposed to elevated humidity (or elevated temperature, or combination thereof), or to solvent vapors for extended period of days.
API is exposed to elevated humidity (or elevated temperature, or combination thereof), or to solvent vapors for extended period of days.
Two or more polymorphs of the API are mixed in a solvent or solvent systems (some solvent mixtures containing variable amount of water) to obtain a slurry, and the slurry will be agitated (at various temperatures) for an extended period of time (days). The solvent system used can be pre-saturated with the API. The final solids will be filtered and dried (air dried or vacuum dried).
API is heated to a specific temperature and cooled (at ambient conditions or in a dry box).

The solids obtained are analyzed by XRPD to determine if they are crystalline and, if so, by DSC to see the melting point and by TG to see if they are hydrated/solvated, and by¹H NMR spectroscopy to ensure chemical integrity. KF water titration is performed on forms that are hydrated. DVS analysis is performed to evaluate hygroscopicity of the form and if hydrated form is present. In particular variable temperature analyses, including variable temperature XRPD, are performed to assess the stability of each physical form as well as its crystallinity.

Differential scanning calorimetry (DSC) thermograms are obtained using a DSC Q 100 (TA Instruments, New Castle, Del.). The temperature axis and cell constant of the DSC cell are calibrated with indium (10 mg, 99.9% pure, melting point 156.6° C., heat of fusion 28.4 J/g). Samples (2.0-5.0 mg) are weighed in aluminum pans on an analytical balance. Aluminum pans without lids are used for the analysis. The samples are equilibrated at 25° C. and heated to 250-300° C. at a heating rate of 10° C./min under continuous nitrogen flow. TG analysis of the samples is performed with a Q 50 (TA Instruments, New Castle, Del.). Samples (2.0-5.0 mg) are analyzed in open aluminum pans under a nitrogen flow (50 mL/min) at 25° C. to 210° C. with a heating rate of 10° C./min.

The sample for moisture analysis is allowed to dry at 25° C. for up to 4 hours under a stream of dry nitrogen. The relative humidity is then increased stepwise from 10 to 90% relative humidity (adsorption scan) allowing the sample to equilibrate for a maximum of four hours before weighing and moving on to the next step. The desorption scan is measured from 85 to 0% relative humidity with the same equilibration time. The sample is then dried under a stream of dry nitrogen at 80° C. for 2 hours or until no weight loss is observed.

X-ray powder diffraction data are collected using a Miniflex Tabletop XRD system (Rigaku/MSC, The Woodlands, Tex.) from 5° to 45 °2θ with steps of 0.1°, and the measuring time is 1.0 second/step. All samples are ground to similar size before exposure to radiation. The powder samples are illuminated using CuKα radiation (λ=1.54056 Å) at 30 kV and 15 mA.

Variable temperature XRPD data are collected using a Huber Imaging Plate Guinier Camera 670 employing Ni-filtered CuKα₁ radiation (λ=1.5405981 Å) produced at 40 kV and 20 mA by a Philips PW1120/00 generator fitted with a Huber long fine-focus tube PW2273/20 and a Huber Guinier Monochromator Series 611/15. The original powder is packed into a Lindemann capillary (Hilgenberg, Germany) with an internal diameter of 1 mm and a wall thickness of 0.01 mm. The sample is heated at an average rate of 5 Kmin⁻¹ using a Huber High Temperature Controller HTC 9634 unit with the capillary rotation device 670.2. The temperature is held constant at selected intervals for 10 min while the sample is exposed to X-rays and multiple scans were recorded. A 20-range of 4.00-100.0° is used with a step size of 0.005 °2θ.

In certain embodiments wherein the solid form is a solvate, such as a hydrate, the DSC thermogram reveals endothermic transitions. In accordance with the observed DSC transitions, TGA analysis indicates stages of weight change corresponding to desolvation or dehydration and/or melting of the sample. In the case of hydrates, these results are in harmony with Karl Fisher titration data which indicate the water content of the sample.

The moisture sorption profile of a sample can be generated to assess the stability of a solid form is stable over a range of relative humidities. In certain embodiments, the change in moisture content over 10.0 to 95.0% relative humidity is small. In other embodiments the change in moisture content over 10.0 to 95.0% relative humidity is reversible.

In certain embodiments, the XRPD pattern of a sample of solid form indicates that the sample has a well-defined crystal structure and a high degree of crystallinity.

### Example 4—Polymorph Screen of 2C-B HCl

### Summary

A polymorph screen of 4-bromo-2,5-dimethoxyphenethylamine HCl (2C-B HCl) was conducted in order to evaluate the polymorphic landscape and identify the solid form for clinical development. Three crystalline polymorphs were identified and designated as Forms A, B, and C. Form A and Form B appear to be anhydrous. Form A was obtained from the vast majority of the crystallization experiments. Form B was observed out of alcohol-containing solvent systems, often as a mixture with Form A. Form C is suspected to be a hemi-hydrate.

Form A is more thermodynamically stable than Form B under conditions tested in this study at ambient temperature and 50° C. This relationship was established by results from competitive slurry experiments.

Because Form C appears to be a hydrate, its stability related to Form A should depend on the water activity of its environment. The water activity boundary between Forms A and C was found to be between 0.5 and 0.6. This was determined from slurry experiments conducted using Form A in systems at various water activities at ambient temperature. Form C was produced at water activities of 0.6 and above, while Form A remained unchanged at water activities of 0.5 and below.

### Background

A polymorph screen of 4-bromo-2,5-dimethoxyphenethylamine HCl (2C-B.HCl) for clinical development is performed. 2C-B is a DEA Schedule I controlled substance and a potent material.

The goals of this study were to identify and characterize crystalline polymorphs of 2C-B HCl and of those, determine which is the most thermodynamically stable form.

### Results and Discussion

### Characterization of 2C-B HCl (Form A)

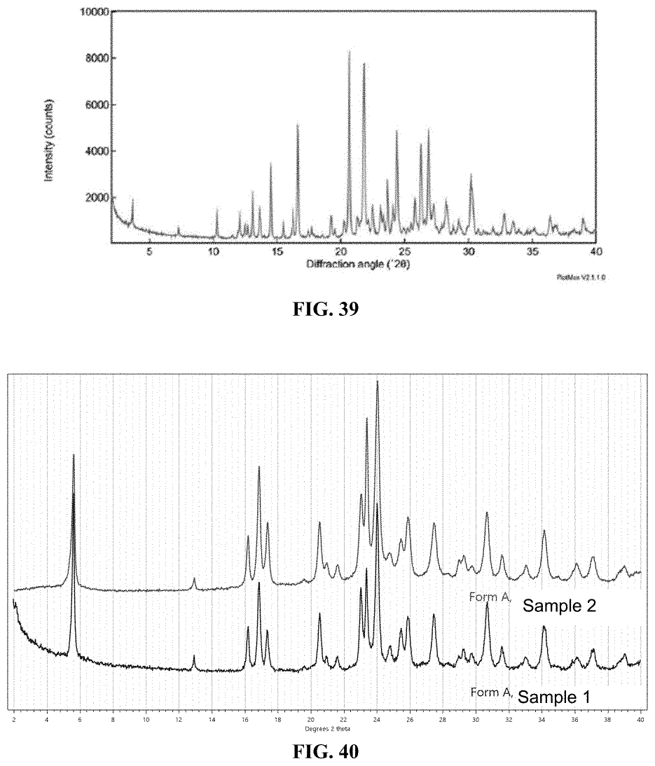

Two samples of 2C-B HCl were characterized by X-ray powder diffraction (XRPD). The lots are both crystalline and composed of the same form, which was designated as Form A (FIG. 40). Lot 0631413 was further characterized by proton nuclear magnetic resonance spectroscopy (¹H NMR), thermogravimetric analysis (TGA), differential scanning calorimetry (DSC), dynamic vapor sorption (DVS) with XRPD of the post-DVS material, optical microscopy (OM), and infrared spectroscopy (IR). Characterization data are summarized in Table 49.

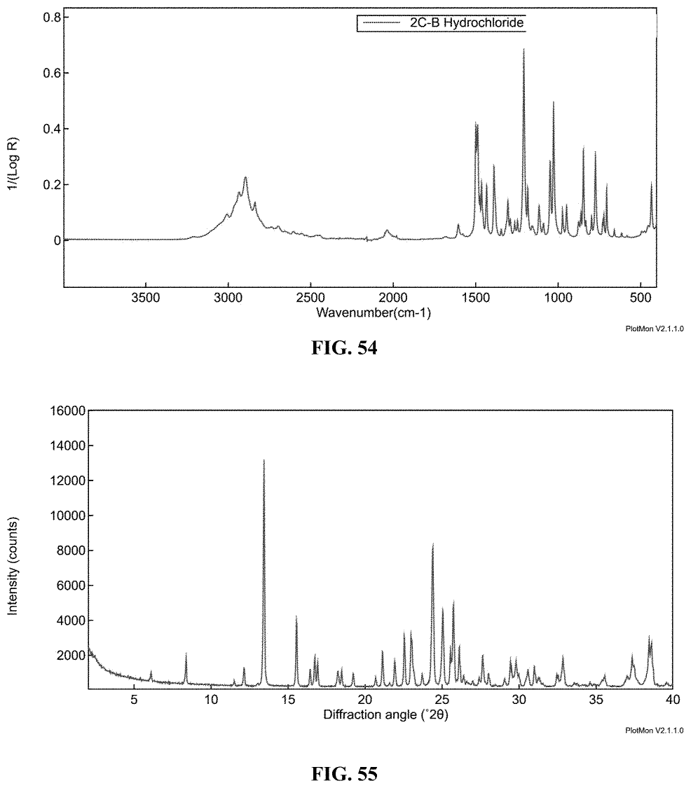

Based on the data, Form A appears to be anhydrous as there are no organic solvents in the NMR spectrum and the TG thermogram shows only a negligible weight loss. The sharp endotherm at 238° C. in the DSC thermogram is likely due to melting. Based on DVS data, Form A is slightly hygroscopic but loses all the gained moisture without form change. The IR spectrum of the 2C-B.HCl (Form A) is provided in FIG. 54.

TABLE 49
Characterization of 2C-B HCl

| | Analytical | | |
|---|---|---|---|
| Sample | Technique | Results | FIG. No. |
| 1 | XRPD | Form A | |
| 2 | XRPD | Form A | |
| | ¹H NMR | Consistent with structure | 45 |
| | (DMSO-d₆) | No organic solvents | |
| | | Small unidentified | |
| | | peak at 4.1 ppm | |
| | TGA | 0.1% start to 155° C. | 50 |
| | DSC | Endo: 90° C., 238° C. | |
| | | (onset 236° C., | |
| | | ΔH°_{fus} = | |
| | | 120.4 J/g) | |
| | DVS | Sorption: | 53 |
| | | 5-95% RH: 0.9% gain | |
| | | Desorption: | |
| | | 95-5% RH: 1.0% loss | |
| | post-DVS | Form A | |
| | XRPD | | |
| | OM | Unknown morphology, aggregates | |
| | | Minor birefringence observed | |
| | IR | Consistent with the literature | 54 |
| | | spectrum* | |

*Giroud C., Rivier L. “2C-B: A New Psychoactive Phenethylamine Recently Discovered in Ecstasy Tablets Sold on the Swiss Black Market.” Journal of Analytical Toxicology. October 1998. DOI: 10.1093/jat/22.5.345

Solubilities of 2C-B HCl in a few solvents were estimated. The experiments were carried out by adding the test solvent in aliquots to weighed portions of solid. Whether dissolution had occurred was judged by visual inspection after addition of each solvent aliquot. The results are shown in Table 50. Solubility numbers were calculated by dividing the weight of the sample by the total amount of solvent used to dissolve the sample. The actual solubilities may be greater than the numbers calculated because of the use of solvent aliquots that were too large or because of slow dissolution rates. Solubilities are reported as “≥” if complete dissolution occurred upon the first solvent aliquot. Values are reported as “≤” if dissolution did not occur during the experiment. All solubility measurements were carried out at room temperature unless noted otherwise.

TABLE 50
Estimated Solubilities of 2C-B HCl

| | Solubility |
|---|---|
| Solvent^{a} | (mg/mL) |
| Acetone | <2 |
| Acetone/H₂O 80/20 | ≥79 |
| ACN | <1 |
| EtOAc | <2 |
| EtOH | 13 |
| H₂O | 34 |
| IPA | <2 |
| MeOH | 41 |
| MeOH/CHCl₃ 50/50 | 41 |
| THF | <2 |

^{a}ACN = acetonitrile; H₂O = water; EtOAc = ethyl acetate; EtOH = ethanol; IPA = isopropanol; MeOH = methanol; CHCl₃ = chloroform; THF = tetrahydrofuran

### Polymorph Screening

2C-B HCl was mixed with various solvents under various conditions in attempts to generate polymorphs. Samples generated and analyzed are listed in Table 3.

(a)

2C-B free base was also used as an alternative starting material for crystallization of the HCl salt. This was done in order to do in-situ experiments with hydrochloric acid as these types of experiments can provide access to polymorphs of the HCl salt that would not otherwise be obtained. Salt breaking experiments are described in Table 52 and in-situ experiments for the polymorph screen are summarized in Table 53. Note that generated samples of free base were poorly crystalline. The XRPD patterns only had a few crystalline peaks and contained a significant amount of non-crystalline material, i.e., diffuse scattering and broad haloes.

TABLE 52
Preparation of Free Base

| | XRPD |
|---|---|
| Conditions^{a} | Results^{b} |
| Added 343 μL of 1N NaOH to a solution of | FB 1 + 2; LC |
| 101.5 mg of 2C-B HCl in ~5-6 mL water | |
| (cloudy, then oiling observed). Extracted w/ | |
| DCM and EtOAc. Combined organic layers and | |
| dried w/MgSO₄. Evaporated w/stream of air. | |
| Added 1,091 μL of 1N NaOH to a solution of | — |
| 215.8 mg of 2C-B HCl in ~3-4 mL water (oil). | |
| Stirring, RT, 30 minutes. Extracted w/EtOAc | |
| (3x). Combined organic layers and dried w/MgSO₄. | |
| Evaporated w/stream of air. | |
| Added 15.2 mL of 1N NaOH to a solution of | FB 1; LC |
| 3.0 g of 2C-B HCl in ~60 mL water (oil). | |
| Stirring, RT, 1 d. Extracted w/EtOAc (3x). | |
| Combined organic layers and dried w/MgSO₄. | |
| Evaporated w/stream of air. | |

^{a}NaOH = sodium hydroxide; H₂O = water; EtOAc = ethyl acetate; DCM = dichloromethane; MgSO₄ = magnesium sulfate; RT = room/ambient temperature; d = day(s)
^{b}FB = free base; LC = low crystallinity

TABLE 53
Samples Generated and Analyzed using 2C-B free base and HCl

| | | | XRPD |
|---|---|---|---|
| Method | Solvent^{a} | Conditions^{b} | Results |
| Slurry | ACN | RT, 6 days | A |
| | DCE | RT, 6 days | A |
| | Dioxane | RT, 6 days | A |
| | Heptane/EtOH | RT, 6 days | A |
| | IPA | RT, 6 days | A + B |
| | IPE | 60° C., 1 day→RT, 5 | A |
| | | days | |
| | iPrOAc | RT, 6 days | A |
| | 2-Me THF | RT, 6 days | A |
| | MEK | RT, 6 days | A |
| | MTBE | 60° C., 1 day→RT, 5 | A + trace C |
| | | days | |

^{a}ACN = acetonitrile; DCE = dichloroethane; EtOH = ethanol; IPA = isopropanol; IPE = di-isopropyl ether; MEK = methyl ethyl ketone; 2-Me THF = 2-methyl tetrahydrofuran; iPrOAc = isopropyl acetate; MTBE = methyl tert-butyl ether;
^{b}RT = room/ambient temperature

In addition to Form A, two polymorphs of 2C-B HCl were identified during screening. They were designated as Form B and Form C. An XRPD overlay of the forms is shown below in FIG. 42.

Form A was observed from the majority of polymorph screen experiments. Form B was observed from alcohol-containing solvent systems, and was typically isolated as a mixture with Form A. Form C was mostly obtained from aqueous systems.

Based on the data in Table 50, Form A is crystalline and appears to be unsolvated and anhydrous. Melting is suspected to occur at 238° C. The TG thermogram shows a weight loss prior to the melting event, suggesting that the material sublimes starting at about 200° C.

Form B and Form C were further characterized by NMR, TGA, and DSC. Data are summarized in Table 54.

TABLE 54
Characterization of Samples

| | Analytical | | |
|---|---|---|---|
| Sample | Technique | Results^{a} | FIG. No. |
| Form B | ¹H NMR | Consistent with structure | 46 |
| | | No organic solvents | |
| | TGA | 0.4% start to 144° C. | FIG. 51 |
| | DSC | Endo: 240° C. (onset 239° C., | |
| | | ΔH°_{fus;} = 122.3 J/g) | |
| Form C | ¹H NMR | Consistent with structure | 47 |
| | | No organic solvents | |
| | TGA | 4.2% start to 86° C. | 52 |
| | DSC | Endo: 73° C., 238° C. (onset 236° C., | |
| | | ΔH°_{fus} = 105.8 J/g) | |
| FB 1 | ¹H NMR | Consistent with structure | 48 |
| | | Significant peak shifts | |
| | | observed for peaks | |
| | | attributable to methylene protons | |
| | | Small unidentified peaks | |
| | | at 3.1 and 2.6 ppm | |
| FB 1 | ¹H NMR | Consistent with structure | 49 |
| | | Significant peak shifts | |
| | | observed for peaks | |
| | | attributable to methylene protons | |
| | | Small unidentified peaks | |
| | | at 3.1 and 2.6 ppm | |

^{a}¹H NMR = proton nuclear magnetic resonance spectroscopy; TGA = thermogravimetric analysis; DSC = differential scanning calorimetry; endo = endotherm; exo = exotherm; FB = free base

Based on the data, Form B appears to be anhydrous as there are no organic solvents by NMR and the TG thermogram shows only a small weight loss (0.4%). The sharp endotherm observed at 240° C. by DSC is typical of melting. Based on the weight loss in the TG prior to the melt, Form B also sublimes beyond 200° C.

Form A was found to be more thermodynamically stable than Form B between ambient and 50° C., based on results from competitive slurry experiments. Typically, competitive slurry experiments are conducted by suspending mixtures of different forms in a solvent pre-saturated with the compound of interest. The more stable form is expected to result from the initial mixture of forms by dissolution-recrystallization. For these experiments, mixtures of Form A and B were slurried in solvents pre-saturated with 2C-B HCl. Note that the slurries were carried out at both ambient and 50° C. because the relative thermodynamic relationship can change with temperature. Experiments and results are described in Table 55.

TABLE 55
Competitive Slurry Experiments with Forms A and B

| | | XRPD | |
|---|---|---|---|
| Solvent | Conditions^{a} | Results | FIG. No. |
| EtOAc | Slurry, RT | A | |
| EtOH | Slurry, RT | A | (FIG. 43, |
| | | | Table 58) |
| IPA | Slurry, RT | A | |
| MEK | Slurry, 50° C. | A | |
| MTBE/MeOH | Slurry, RT | A | |

^{a}EtOAc = ethyl acetate; EtOH = ethanol; IPA = isopropanol; MEK = methyl ethyl ketone; MTBE = methyl tert-butyl ether; MeOH = methanol; RT = room/ambient temperature

Form C is suspected to be hydrated. The TG thermogram shows a 4.2% weight loss from ambient to 86° C., which is accompanied by a broad endotherm in the DSC thermogram. Because there are no organic solvents in the NMR spectrum, this is likely due to water and would be equivalent to approximately 0.5 moles. The sharp endotherm at 238° C. in the DSC thermogram is typical of melting. Note that this occurs at the same temperature as Form A and could indicate Form C converts to Form A upon heating. The same sublimation event seen in the TG of Form A was also observed in the TG for Form C (weight loss prior to the final melting endotherm).

Because Form C appears to be a hydrate, its stability related to Form A should depend on the water activity of the crystal's environment. Typically, such a system has a water activity boundary, above which the hydrate is preferred and below which the anhydrate is preferred. Note that the hydrate-anhydrate relationship is not one of relative thermodynamic stability because the species are different rather than polymorphic.

Water activity studies were conducted to determine the boundary between Form A and Form C. For these experiments, 2C-B HCl Form A was suspended in solvents containing different amounts of water, thus having different water activities. Note that the slurries were carried out at ambient temperature and, like with relative stability, the water activity boundary can change with temperature. Experiments and results are summarized in Table 56.

TABLE 56
Water Activity Experiments

| | | XRPD |
|---|---|---|
| Solvent^{a} | Conditions | Results |
| ACN/H₂O 99/1 | Slurry, RT, 7 days. Solids | A |
| a_{w} = 0.21 | analyzed wet. | |
| THF/H₂O 98/2 | Slurry, RT, 7 days. Solids | A |
| a_{w} = 0.38 | analyzed wet. | |
| ACN/H₂O 97/3 | Slurry, RT, 7 days. Solids | A |
| a_{w} = 0.50 | analyzed wet. | |
| ACN/H₂O 96/4 | Slurry, RT, 6 days. Solids | C |
| a_{w} = 0.60 | analyzed wet. | |
| ACN/H₂O 94/6 | Slurry, RT, 6 days. Solids | C |
| a_{w} = 0.73 | analyzed wet. | |
| THF/H₂O 94/6 | Slurry, RT, 6 days. Solids | C |
| a_{w} = 0.84 | analyzed wet. | |
| THF/H₂O 90/10 | Slurry, RT, 6 days. Solids | C |
| a_{w} = 1.00 | analyzed wet. | |

^{a}Water activity values are predicted values for the solvent system only. They are not representative of the solvent with solids.
(b) ACN = acetonitrile; THF = tetrahydrofuran; H₂O = water; a_{w} = water activity; RT = room/ambient temperature

Form C was obtained at water activities of 0.6 and above. Form A remained unchanged at water activities of 0.5 and below. This means that the boundary of conversion between the two forms is between 0.5 and 0.6 water activity.

It is important to realize that the water activity boundary was established in a wet system where dissolution-recrystallization occurs. Those results cannot be translated to the solid state, in which such conversions are typically slower than in a slurry. Therefore, it is possible that crystalline Form C could be stored for an extended period of time under low humidity conditions (below ˜50% RH) without conversion to Form A. Likewise, it is possible that Form A could be stored for an extended period of time at high humidity (above ˜50% RH) without conversion to Form C. Additional studies would be necessary to determine the rates of hydrate-to-anhydrate conversion, or the reverse, in the solid state. It should be mentioned that some samples generated during screening displayed significant preferred orientation (PO) in the XRPD pattern. Preferred orientation can increase or decrease peak intensities such that the peaks seem to appear and/or disappear between different patterns of the same form. A more extreme example from this study is shown below in FIG. 44. While the patterns are all Form A, the blue pattern only contains a few peaks and is missing several characteristic peaks of Form A. However, after rendering and re-analyzing the sample, the PO was eliminated, resulting in a more typical pattern of Form A (green pattern).

### Conclusions

A polymorph screen of 2C-B HCl was conducted. Three crystalline polymorphs were identified and designated as Forms A, B, and C. Form A and Form B both appear to be anhydrous while Form C is suspected to be a hemi-hydrate. Form A was obtained from the majority of screening experiments. Form B was observed from alcohol-containing solvent systems. Under conditions tested in this study, Form A is more thermodynamically stable than Form B.

Because Form C is a hydrate, its stability relative to Form A is not a thermodynamic relationship but instead depends on the water activity of its environment. The boundary between Forms A and C was established to be between 0.5 and 0.6 water activity in slurry conditions at ambient temperature. Form C was obtained from systems with water activities of 0.6 and above, while Form A remained unchanged from systems with water activities of 0.5 and below.

### Experimental

### Polymorph Screen

### Typical Evaporation Experiment

To approximately 15 mg of 2C-B HCl in a 1-dram vial was added 1.1 mL of Et0H, resulting in a clear solution. The solution was filtered through a 0.45 μm nylon filter into a new vial. The vial was covered with aluminum foil with 1 pinhole and left undisturbed at ambient temperature. Upon evaporation to dryness, the resulting solid was collected and analyzed by XRPD to produce Form B (containing some Form A) material.

### Typical Slurry Experiment

To approximately 20 mg of 2C-B HCl was added 800 μL of CHCl₃. The resulting suspension was stirred at ambient temperature for approximately 2 weeks. The slurry was then centrifuged, the mother liquor was decanted, and the isolated solid was air-dried and analyzed by XRPD to produce Form A material.

### Typical Cooling Experiment

To approximately 20 mg of 2C-B HCl was added approximately 5 mL of MTBE. The resulting suspension was heated at 60° C. Approximately 2 mL of MeOH was added at elevated temperature, producing a hazy solution which was then hot-filtered into a pre-heated vial. Upon cooling to ambient temperature, precipitation was observed. The mother liquor was decanted and the isolated solid was analyzed by XRPD to produce a mixture of Form A and Form B.

If precipitation was not observed upon cooling to ambient temperature, the solution was transferred to a refrigerator, resulting in a solid. The mother liquor was decanted and the isolated solid was analyzed by XRPD.

### Typical Precipitation Experiment

### Method 1

To approximately 20 mg of 2C-B HCl was added approximately 500 μL of EtOH. The resulting suspension was heated at 60° C., resulting in a clear solution. The solution was hot-filtered into approximately 5 mL of hexanes at ambient temperature. Precipitation was not observed and the solution was transferred to a refrigerator, resulting in a solid. The mother liquor was decanted and the isolated solid was analyzed by XRPD to produce a mixture of Form A and Form B.

### Method 2

To approximately 20 mg of 2C-B HCl was added a minimal amount of MeOH, resulting in a clear solution. Approximately 5 mL of DEE was added at ambient temperature. Upon standing at ambient temperature, precipitation was observed. The mother liquor was decanted and the isolated solid was analyzed by XRPD to produce Form A material.

Note that for some experiments, precipitation was not observed upon cooling to ambient temperature and the solution was transferred to a refrigerator for further cooling, resulting in a solid. The mother liquor was decanted and the isolated solid was analyzed by XRPD.

### Typical Vapor Diffusion Experiment

To approximately 20 mg of 2C-B HCl in a 1-dram vial was added a minimal amount of MeOH, resulting in a clear solution. The 1-dram vial was placed, uncapped, into a 20 mL vial containing approximately 5 mL of MTBE. The vial was capped and left undisturbed at ambient temperature. After standing at ambient temperature for several days, precipitation was observed. The resulting solid was analyzed by XRPD to produce Form B (containing some Form A) material.

If precipitation was not observed, the sample was transferred to a refrigerator for further cooling or evaporated at ambient temperature.

### Typical Competitive Slurry Experiment

To solids of 2C-B HCl (sample TCL16247) was added EtOAc. The resulting suspension was stirred at ambient temperature for 1 day. The slurry was centrifuged and the mother liquor was decanted.

The mother liquor was then added to approximately equal amounts (visual) of 2C-B HCl Form A and Form B and the resulting suspension was stirred at ambient temperature for several days. The slurry was then centrifuged, the mother liquor was decanted, and the isolated solid was analyzed by XRPD to produce Form A material.

### Typical Water Activity Slurry Experiment

To approximately 40 mg of 2C-B HCl (sample TCL16247) was added 2 mL of an ACN/H₂O mixture (97/3 v/v). The resulting suspension was stirred at ambient temperature for 1 week. The slurry was then centrifuged, the mother liquor was decanted, and the isolated solids were analyzed by XRPD to produce Form A material.

### Preparation of Form B

To approximately 15 mg of 2C-B HCl was added 6.2 mL of IPA, resulting in a hazy suspension. The sample was then stirred at 50° C., during which complete dissolution occurred. The clear solution was hot-filtered (0.45 μm nylon) into a pre-heated vial and slowly cooled to ambient temperature. Upon cooling to ambient temperature, precipitation was observed. The solution was decanted and the solid was briefly dried with air. The isolated solid was analyzed by XRPD to produce Form B material.

### Preparation of Form C

To solids of 2C-B HCl was added 1.4 mL of wet EtOAc (EtOAc pre-saturated with water). The resulting suspension was slurried at ambient temperature for 2 weeks. It was then centrifuged and the mother liquor was decanted. The isolated solid was analyzed by XRPD to produce Form C material.

In some embodiments, the characterization data of 2C-B.HCl (Form B) are as provided in FIG. 41 and Table 57.

TABLE 57
XRPD Signal angle data of 2C-B•HCl (Form B)

| Position (°2θ) | d-value | Relative |
|---|---|---|
| 5.6 | 15.78 | 44 |
| 12.1 | 7.33 | 7 |
| 13.0 | 6.80 | 7 |
| 14.4 | 6.15 | 5 |
| 16.2 | 5.48 | 27 |
| 16.6 | 5.33 | 14 |
| 17.0 | 5.22 | 5 |
| 17.6 | 5.03 | 4 |
| 18.2 | 4.88 | 6 |
| 20.4 | 4.36 | 21 |
| 22.7 | 3.91 | 14 |
| 23.6 | 3.78 | 100 |
| 25.0 | 3.56 | 12 |
| 26.1 | 3.42 | 14 |
| 27.2 | 3.28 | 7 |
| 27.3 | 3.26 | 8 |
| 27.7 | 3.22 | 7 |
| 29.1 | 3.06 | 6 |
| 30.3 | 2.95 | 30 |
| 31.6 | 2.83 | 7 |
| 33.0 | 2.71 | 4 |
| 33.2 | 2.70 | 5 |
| 33.5 | 2.67 | 6 |
| 34.2 | 2.62 | 6 |
| 35.7 | 2.52 | 6 |
| 36.6 | 2.46 | 9 |
| 37.4 | 2.41 | 4 |
| 38.4 | 2.35 | 6 |
| 38.9 | 2.31 | 5 |

In some embodiments, the characterization data of 2C-B HCl (Form A) are as provided in FIG. 43 and Table 58.

TABLE 58
XRPD Signal angle data of 2C-B•HCl (Form A)

| Position (°2θ) | d-value | Relative |
|---|---|---|
| 5.7 | 15.56 | 100 |
| 13.0 | 6.82 | 8 |
| 16.3 | 5.45 | 20 |
| 16.9 | 5.25 | 33 |
| 17.0 | 5.23 | 31 |
| 17.4 | 5.10 | 25 |
| 19.6 | 4.52 | 4 |
| 20.6 | 4.31 | 27 |
| 21.0 | 4.23 | 9 |
| 21.7 | 4.10 | 9 |
| 23.1 | 3.86 | 39 |
| 23.4 | 3.80 | 47 |
| 24.1 | 3.69 | 99 |
| 24.9 | 3.58 | 14 |
| 25.6 | 3.49 | 20 |
| 25.9 | 3.44 | 24 |
| 27.5 | 3.24 | 38 |
| 29.0 | 3.08 | 8 |
| 29.3 | 3.05 | 10 |
| 29.8 | 3.00 | 9 |
| 30.8 | 2.90 | 27 |
| 31.6 | 2.83 | 14 |
| 32.8 | 2.73 | 5 |
| 33.1 | 2.71 | 9 |
| 34.2 | 2.62 | 19 |
| 35.0 | 2.56 | 4 |
| 36.0 | 2.50 | 5 |
| 36.1 | 2.49 | 7 |
| 36.3 | 2.47 | 7 |
| 37.2 | 2.42 | 11 |
| 38.8 | 2.32 | 6 |
| 39.1 | 2.30 | 9 |
| 39.7 | 2.27 | 5 |

### Instrumental Techniques

### X-ray Powder Diffraction (XRPD)

A Rigaku SmartLab X-Ray Diffractometer was configured in Bragg-Brentano reflection geometry equipped with a beam stop and knife edge to reduce incident beam and air scatter. Data collection parameters are shown in the following table.

PXRD Data Collection Parameters

| Parameter | Value | Parameter | Value |
|---|---|---|---|
| Geometry | Bragg-Brentano | Receiving Slit 1 | 18 |
| | | (mm) | |
| Tube Anode | Cu | Receiving Slit 2 | open |
| | | (mm) | |
| Tube Type | Long Fine Focus | Start Angle | 2 |
| | | 2θ (°) | |
| Tube Voltage | 40 | End Angle | 40 |
| (kV) | | 2θ (°) | |
| Tube Current | 44 | Step Size (°) | 0.02 |
| (mA) | | | |
| Detector | D/teX Ultra 250 | Scan Speed | 6 |
| | (RX1) | (°/min) | |
| | HyPix-3000 (RX4) | | |
| Monochromator | Ni foil Cu Kβ Filter | Spinning (rpm) | 11 |
| Incident | ⅓ | Sample Holder | Low- |
| Slit (°) | | | background |
| | | | Si |

### Differential Scanning Calorimetry (DSC)

The DSC analyses were carried out using a TA Instruments Q2500 Discovery Series instrument. The instrument temperature calibrations were performed using indium. The DSC cell was kept under a nitrogen purge of ˜50 mL per minute during the analyses. The samples were placed in a standard, crimped aluminum pan and headed from approximately 25° C. to 300° C. at a rate of 10° C. per minute.

### Thermogravimetric (TG) Analysis

The TG analyses were carried out using a TA Instruments Q50 Discovery Series instrument. The nitrogen purge was ˜10 mL per minute at the balance and ˜90 mL per minute at the furnace. The samples were placed into a pre-tared platinum pan and heated to approximately 25° C. to 300° C. at a rate of 10° C. per minute.

### Dynamic Vapor Sorption (DVS) Analysis

The DVS analysis was carried out using a TA Instruments Q5000 Dynamic Vapor Sorption analyzer. 13.5 mg of sample was loaded into a metal-coated quartz pan for analysis. After equilibration to 5% relative humidity (RH), the sample was analyzed at 25° C. in 10% RH steps from 5 to 95% RH (adsorption cycle) and from 95 to 5% RH (desorption cycle). The movement from one step to the next occurred either after satisfying the equilibrium criterion of 0.01% weight change in 5 minutes or, if the equilibrium criterion was not met, after 90 minutes. The percent weight change values are calculated using Microsoft Excel®.

### Nuclear Magnetic Resonance (NMR) Spectroscopy

The ¹H NMR spectra were acquired on a Bruker Avance II 400 spectrometer. Samples were prepared by dissolving material in DMSO-d₆. The solutions were placed into individual 5-mm NMR tubes for subsequent spectral acquisition. The temperature controlled (295K)¹H NMR spectra acquired on the Avance II 400 utilized a 5-mm cryoprobe operating at an observing frequency of 400.18 MHz.

### Infrared (IR) Spectroscopy

The IR spectrum was obtained on a Nicolet 6700 FT-IR system. The sample was analyzed using a Nicolet SMART iTR attenuated total reflectance device.

### Example 5—Salt Screen of 2C-B and Polymorph Screen of a Selected Salt

### Summary

A salt screen of 4-bromo-2,5-dimethoxyphenethylamine (2C-B) free base was conducted. The goal of the study was to identify a salt suitable for clinical development.

Salt screen results are highlighted below;
Crystalline salts were generated with 2C-B free base and the following acids: benzenesulfonic acid, citric acid, ethanesulfonic acid, fumaric acid, gentisic acid, glutamic acid, glycolic acid, D,L-lactic acid, L-malic acid, maleic acid, methanesulfonic acid, mucic (galactaric) acid, phosphoric acid, succinic acid, sulfuric acid, L-tartaric acid, toluenesulfonic acid, and 1-hydroxy-2-naphthoic (xinafoic) acid.
A potential salt was observed from experiments with L-aspartic acid but was suspected to be a mixture. The sample was not further characterized and salt formation was not confirmed.
A crystalline material with 1:5.5 (API:acid) stoichiometry was produced with malonic acid. Formation of a stoichiometric salt is unlikely, but further study would be needed to confirm the nature of this material.
Selected salts were made at larger scale and further characterized. Their properties are summarized in the following table. Several of these salts appear potentially viable for clinical development; however, the tartrate salt was ultimately recommended for additional investigation on the basis of its physical properties.

A summary id provided in Table 59.

TABLE 59
Summary Table

| | | Suspected | | | |
|---|---|---|---|---|---|
| | | solvation/ | | | |
| | | hydration | Suspected | | |
| Salt | Stoichiometry | state | Melt | Hygroscopicity | Comments |
| Besylate | 1:1 | anhydrous | 203° C. | Slightly | 2 forms observed |
| (Form 1) | | | | hygroscopic; | Reproduced at 0.5 g |
| | | | | 5-95% RH: 0.7% | 5 mg/mL aqueous |
| | | | | gain | solubility (kinetic) |
| | | | | Did not deliquesce | |
| | | | | at 75% RH | |
| Citrate | 2:1 | anhydrous | 176° C. | Slightly | 2 forms observed |
| (Form 2) | (API:acid) | | | hygroscopic; | Produced from |
| | | | | 5-95% RH: 0.5% | scale-up attempt of |
| | | | | gain | Citrate (Form 1) |
| | | | | Did not deliquesce | 10 mg/mL aqueous |
| | | | | at 75% RH | solubility (kinetic) |
| Gentisate | 1:1 | anhydrous | 214° C. | Non-hygroscopic; | 1 form observed |
| | | | | 5-95% RH: 0.2% | Reproduced at 0.5 g |
| | | | | gain | 2 mg/mL aqueous |
| | | | | Did not deliquesce | solubility (kinetic) |
| | | | | at 75% RH | |
| Glycolate | 1:1 | anhydrous | 149° C. | Mod. hygroscopic; <85% | 1 form observed |
| | | | | RH: 0.9% | Reproduced at 0.5 g |
| | | | | gain >85% | ≥45 mg/mL aqueous |
| | | | | RH: 2.7% gain | solubility (kinetic) |
| | | | | Did not deliquesce | |
| | | | | at 75% RH | |
| Malate | 1:1 | anhydrous | 152° C. | Slightly | Reproduced at 0.5 g |
| | | | | hygroscopic; <85% | XRPD pattern indexed |
| | | | | RH: 0.6% | Additional peaks in |
| | | | | gain >85% | XRPD pattern of |
| | | | | RH: 1.0% gain | original screen |
| | | | | Did not deliquesce | sample; not allowed |
| | | | | at 75% RH | per indexing |
| | | | | | sol'n→suspected |
| | | | | | polymorph |
| | | | | | 6 mg/mL aqueous |
| | | | | | solubility (kinetic) |
| Mesylate | 1:1 | anhydrous | 179° C. | Hygroscopic; <85% | 1 form observed |
| | | | | RH: 2.7% | Reproduced at |
| | | | | gain >85% | 0.5 g scale |
| | | | | RH: 4.5% gain | ≥38 mg/mL |
| | | | | Did not deliquesce | aqueous |
| | | | | at 75% RH | solubility |
| | | | | | (kinetic) |
| Sulfate | 1:1 (IC) or | anhydrous | 169° C. | Slightly | 1 form observed |
| | 2:3 (EDX) | | | hygroscopic; <85% | Reproduced at 0.5 g |
| | | | | RH: 0.8% | 14 mg/mL aqueous |
| | | | | gain >85% | solubility |
| | | | | RH: 1.4% | (kinetic) |
| | | | | gain | 36.1% SO₄⁻² |
| | | | | Did not deliquesce | content (theoretical for |
| | | | | at 75% RH | 2:3: 35.4%) |
| | | | | | Bromine:Sulfur |
| | | | | | ratio 1:0.8 |
| Tartrate | 1:1 | anhydrous | 206° C. | Non-hygroscopic; | 1 form observed |
| (Form 1) | | | | 5-95% RH: 0.1% | Reproduced at 0.5 g |
| | | | | gain | 11 mg/mL aqueous |
| | | | | Did not deliquesce | solubility |
| | | | | at 75% RH | (kinetic) |

In some embodiments, the characterization data of 2C-B B.esylate (Form 1) are as provided in FIG. 55 and Table 61.

TABLE 61
XRPD Signal angle data of 2C-B Besylate (Form 1)

| Position (°2θ) | d-value | Relative |
|---|---|---|
| 6.1 | 14.49 | 8 |
| 8.4 | 10.55 | 14 |
| 11.5 | 7.69 | 4 |
| 12.1 | 7.29 | 10 |
| 13.4 | 6.59 | 100 |
| 15.5 | 5.70 | 31 |
| 16.4 | 5.39 | 9 |
| 16.7 | 5.30 | 15 |
| 16.9 | 5.24 | 12 |
| 18.2 | 4.86 | 8 |
| 18.5 | 4.80 | 9 |
| 19.2 | 4.62 | 7 |
| 20.7 | 4.30 | 6 |
| 21.1 | 4.21 | 17 |
| 21.6 | 4.12 | 3 |
| 21.9 | 4.05 | 13 |
| 22.5 | 3.94 | 24 |
| 23.0 | 3.87 | 24 |
| 23.7 | 3.75 | 7 |
| 24.4 | 3.65 | 63 |
| 25.0 | 3.56 | 35 |
| 25.5 | 3.49 | 18 |
| 25.7 | 3.46 | 38 |
| 26.1 | 3.41 | 19 |
| 26.4 | 3.38 | 7 |
| 26.6 | 3.35 | 4 |
| 27.0 | 3.30 | 4 |
| 27.4 | 3.26 | 6 |
| 27.6 | 3.23 | 15 |
| 28.0 | 3.18 | 7 |
| 29.1 | 3.07 | 5 |
| 29.4 | 3.03 | 13 |
| 29.5 | 3.03 | 10 |
| 29.8 | 3.00 | 13 |
| 30.0 | 2.98 | 7 |
| 30.6 | 2.92 | 9 |
| 31.0 | 2.89 | 11 |
| 31.3 | 2.86 | 6 |
| 32.4 | 2.76 | 7 |
| 32.5 | 2.75 | 7 |
| 32.8 | 2.73 | 14 |
| 33.6 | 2.67 | 3 |
| 33.7 | 2.66 | 3 |
| 34.6 | 2.59 | 4 |
| 35.4 | 2.53 | 5 |
| 35.6 | 2.52 | 6 |
| 37.0 | 2.43 | 6 |
| 37.3 | 2.41 | 15 |
| 37.5 | 2.40 | 11 |
| 38.4 | 2.34 | 22 |
| 38.6 | 2.33 | 21 |
| 39.6 | 2.28 | 4 |

In some embodiments, the characterization data of 2C-B Gentisate are as provided in FIG. 56 and Table 62.

TABLE 62
XRPD Signal angle data of 2C-B Gentisate

| Position (°2θ) | d-value | Relative |
|---|---|---|
| 10.1 | 8.76 | 19 |
| 11.1 | 7.99 | 8 |
| 12.0 | 7.39 | 4 |
| 12.4 | 7.12 | 4 |
| 13.1 | 6.75 | 36 |
| 14.3 | 6.21 | 14 |
| 15.0 | 5.91 | 7 |
| 15.5 | 5.70 | 10 |
| 16.5 | 5.36 | 33 |
| 17.2 | 5.14 | 26 |
| 18.9 | 4.71 | 14 |
| 19.2 | 4.62 | 8 |
| 19.9 | 4.45 | 8 |
| 20.2 | 4.40 | 20 |
| 20.5 | 4.33 | 22 |
| 20.9 | 4.26 | 18 |
| 22.2 | 4.00 | 100 |
| 22.8 | 3.91 | 10 |
| 23.6 | 3.77 | 19 |
| 23.8 | 3.74 | 25 |
| 24.3 | 3.67 | 22 |
| 24.9 | 3.57 | 13 |
| 25.2 | 3.54 | 20 |
| 25.6 | 3.48 | 94 |
| 26.4 | 3.38 | 33 |
| 26.6 | 3.35 | 18 |
| 27.3 | 3.27 | 11 |
| 28.6 | 3.12 | 21 |
| 29.3 | 3.05 | 6 |
| 30.1 | 2.97 | 22 |
| 30.4 | 2.94 | 10 |
| 31.1 | 2.88 | 8 |
| 31.3 | 2.86 | 7 |
| 32.0 | 2.80 | 9 |
| 32.1 | 2.79 | 10 |
| 32.4 | 2.76 | 8 |
| 32.8 | 2.73 | 17 |
| 33.1 | 2.71 | 12 |
| 33.7 | 2.66 | 12 |
| 34.3 | 2.62 | 10 |
| 34.6 | 2.59 | 12 |
| 34.8 | 2.58 | 10 |
| 35.5 | 2.53 | 14 |
| 36.1 | 2.49 | 6 |
| 36.3 | 2.47 | 7 |
| 36.7 | 2.45 | 13 |
| 37.9 | 2.37 | 13 |

In some embodiments, the characterization data of 2C-B Gentisate (PO) are as provided in FIG. 57 and Table 63.

TABLE 63
XRPD Signal angle data of 2C-B Gentisate (PO)

| Position (°2θ) | d-value | Relative |
|---|---|---|
| 10.1 | 8.78 | 100 |
| 11.1 | 8.00 | 7 |
| 11.9 | 7.41 | 6 |
| 13.1 | 6.75 | 72 |
| 14.3 | 6.21 | 9 |
| 15.0 | 5.91 | 8 |
| 15.5 | 5.71 | 9 |
| 16.5 | 5.37 | 33 |
| 17.2 | 5.15 | 31 |
| 18.9 | 4.71 | 17 |
| 19.2 | 4.62 | 7 |
| 20.2 | 4.40 | 82 |
| 20.5 | 4.33 | 18 |
| 20.9 | 4.26 | 61 |
| 22.0 | 4.04 | 33 |
| 22.2 | 4.00 | 91 |
| 22.5 | 3.95 | 20 |
| 23.6 | 3.77 | 13 |
| 23.7 | 3.76 | 12 |
| 23.8 | 3.74 | 29 |
| 24.1 | 3.70 | 8 |
| 24.3 | 3.67 | 10 |
| 24.9 | 3.58 | 8 |
| 25.2 | 3.54 | 28 |
| 25.6 | 3.48 | 97 |
| 26.4 | 3.38 | 18 |
| 26.6 | 3.35 | 13 |
| 27.3 | 3.27 | 11 |
| 28.6 | 3.12 | 12 |
| 30.1 | 2.97 | 17 |
| 30.5 | 2.93 | 33 |
| 30.7 | 2.92 | 16 |
| 31.1 | 2.88 | 7 |
| 31.9 | 2.81 | 7 |
| 32.1 | 2.79 | 10 |
| 32.4 | 2.76 | 7 |
| 32.7 | 2.74 | 28 |
| 32.8 | 2.73 | 25 |
| 33.1 | 2.71 | 19 |
| 33.7 | 2.66 | 10 |
| 34.3 | 2.61 | 11 |
| 34.6 | 2.59 | 12 |
| 34.8 | 2.58 | 11 |
| 35.5 | 2.53 | 7 |
| 36.1 | 2.49 | 8 |
| 36.3 | 2.47 | 9 |
| 36.7 | 2.45 | 9 |
| 37.9 | 2.37 | 9 |
| 38.2 | 2.35 | 6 |

In some embodiments, the characterization data of 2C-B Glutamate are as provided in FIG. 58 and Table 64.

TABLE 64
XRPD Signal angle data of 2C-B Glutamate

| Position (°2θ) | d-value | Relative |
|---|---|---|
| 3.6 | 24.27 | 53 |
| 7.3 | 12.18 | 29 |
| 12.8 | 6.94 | 13 |
| 14.2 | 6.23 | 15 |
| 14.6 | 6.08 | 14 |
| 16.4 | 5.41 | 42 |
| 16.6 | 5.35 | 25 |
| 16.9 | 5.23 | 35 |
| 17.0 | 5.21 | 33 |
| 18.0 | 4.93 | 19 |
| 19.0 | 4.67 | 12 |
| 20.1 | 4.43 | 16 |
| 20.6 | 4.30 | 18 |
| 21.0 | 4.23 | 18 |
| 22.0 | 4.05 | 100 |
| 23.2 | 3.83 | 16 |
| 23.5 | 3.78 | 52 |
| 24.5 | 3.63 | 37 |
| 24.8 | 3.60 | 30 |
| 24.8 | 3.58 | 31 |
| 25.1 | 3.54 | 24 |
| 25.5 | 3.49 | 74 |
| 27.4 | 3.25 | 18 |
| 27.9 | 3.20 | 20 |
| 28.4 | 3.14 | 23 |
| 29.3 | 3.05 | 11 |
| 29.9 | 2.98 | 21 |
| 30.5 | 2.93 | 22 |
| 31.8 | 2.81 | 31 |
| 32.8 | 2.73 | 14 |
| 33.1 | 2.71 | 17 |
| 33.7 | 2.66 | 24 |
| 34.9 | 2.57 | 17 |
| 35.8 | 2.51 | 21 |
| 36.4 | 2.47 | 15 |
| 39.0 | 2.31 | 20 |

In some embodiments, the characterization data of 2C-B Glycolate are as provided in FIG. 59 and Table 65.

TABLE 65
XRPD Signal angle data of 2C-B Glycolate

| Position (°2θ) | d-value | Relative |
|---|---|---|
| 6.4 | 13.90 | 9 |
| 11.5 | 7.67 | 8 |
| 12.2 | 7.25 | 7 |
| 12.7 | 6.99 | 8 |
| 13.6 | 6.52 | 12 |
| 13.9 | 6.38 | 14 |
| 15.7 | 5.66 | 36 |
| 16.1 | 5.52 | 40 |
| 17.7 | 5.01 | 10 |
| 18.2 | 4.87 | 9 |
| 19.0 | 4.67 | 73 |
| 20.1 | 4.41 | 19 |
| 20.7 | 4.29 | 14 |
| 21.5 | 4.14 | 96 |
| 22.0 | 4.04 | 13 |
| 22.4 | 3.96 | 15 |
| 23.0 | 3.87 | 32 |
| 23.3 | 3.82 | 21 |
| 24.2 | 3.67 | 17 |
| 25.5 | 3.50 | 30 |
| 25.7 | 3.46 | 22 |
| 26.3 | 3.38 | 100 |
| 27.0 | 3.30 | 30 |
| 27.2 | 3.27 | 27 |
| 28.0 | 3.19 | 20 |
| 28.8 | 3.10 | 13 |
| 29.7 | 3.00 | 10 |
| 30.3 | 2.95 | 11 |
| 31.6 | 2.83 | 18 |
| 32.3 | 2.77 | 13 |
| 33.1 | 2.71 | 15 |
| 33.6 | 2.66 | 20 |
| 34.0 | 2.64 | 15 |
| 34.6 | 2.59 | 18 |
| 36.3 | 2.48 | 10 |
| 37.2 | 2.41 | 10 |
| 37.7 | 2.39 | 11 |
| 38.6 | 2.33 | 13 |
| 39.6 | 2.27 | 15 |

In some embodiments, the characterization data of 2C-B Malate are as provided in FIG. 60 and Table 66.

TABLE 66
XRPD Signal angle data of 2C-B Malate

| Position (°2θ) | d-value | Relative |
|---|---|---|
| 10.2 | 8.66 | 6 |
| 12.0 | 7.36 | 31 |
| 14.6 | 6.08 | 12 |
| 16.5 | 5.37 | 44 |
| 17.3 | 5.14 | 8 |
| 17.6 | 5.05 | 28 |
| 17.9 | 4.97 | 45 |
| 18.7 | 4.75 | 14 |
| 19.1 | 4.64 | 17 |
| 19.8 | 4.48 | 50 |
| 20.4 | 4.35 | 26 |
| 21.9 | 4.05 | 23 |
| 23.3 | 3.82 | 43 |
| 23.9 | 3.72 | 100 |
| 24.4 | 3.65 | 51 |
| 25.0 | 3.56 | 24 |
| 25.5 | 3.50 | 40 |
| 25.9 | 3.44 | 7 |
| 26.2 | 3.40 | 15 |
| 27.2 | 3.27 | 15 |
| 27.6 | 3.23 | 11 |
| 29.3 | 3.05 | 9 |
| 29.9 | 2.99 | 14 |
| 30.2 | 2.96 | 8 |
| 30.3 | 2.95 | 7 |
| 30.8 | 2.90 | 22 |
| 31.9 | 2.81 | 13 |
| 33.2 | 2.70 | 9 |
| 33.8 | 2.65 | 17 |
| 34.5 | 2.60 | 11 |
| 34.8 | 2.58 | 35 |
| 35.1 | 2.56 | 15 |
| 35.9 | 2.50 | 6 |
| 36.1 | 2.49 | 13 |
| 36.8 | 2.44 | 5 |
| 37.0 | 2.43 | 7 |
| 37.4 | 2.41 | 5 |
| 37.6 | 2.39 | 10 |
| 37.7 | 2.38 | 10 |
| 38.0 | 2.37 | 9 |
| 38.8 | 2.32 | 13 |
| 39.1 | 2.30 | 11 |
| 39.6 | 2.27 | 6 |
| 39.8 | 2.26 | 6 |

In some embodiments, the characterization data of 2C-B Mesylate are as provided in FIG. 61 and Table 67.

TABLE 67
XRPD Signal angle data of 2C-B Mesylate

| Position (°2θ) | d-value | Relative |
|---|---|---|
| 5.8 | 15.34 | 39 |
| 11.4 | 7.73 | 74 |
| 13.1 | 6.75 | 11 |
| 15.5 | 5.73 | 36 |
| 16.7 | 5.30 | 6 |
| 17.1 | 5.17 | 8 |
| 17.6 | 5.03 | 67 |
| 17.8 | 4.99 | 41 |
| 19.1 | 4.65 | 84 |
| 19.3 | 4.60 | 39 |
| 19.7 | 4.51 | 22 |
| 20.1 | 4.42 | 50 |
| 20.4 | 4.35 | 30 |
| 21.3 | 4.18 | 61 |
| 22.5 | 3.96 | 82 |
| 22.6 | 3.93 | 51 |
| 22.7 | 3.91 | 59 |
| 23.9 | 3.72 | 39 |
| 24.2 | 3.68 | 19 |
| 24.9 | 3.58 | 13 |
| 25.6 | 3.47 | 74 |
| 25.9 | 3.44 | 100 |
| 26.2 | 3.40 | 18 |
| 27.2 | 3.28 | 13 |
| 28.0 | 3.18 | 21 |
| 28.2 | 3.16 | 37 |
| 28.7 | 3.11 | 27 |
| 29.3 | 3.05 | 7 |
| 29.7 | 3.01 | 8 |
| 30.1 | 2.97 | 19 |
| 30.3 | 2.95 | 9 |
| 30.5 | 2.93 | 9 |
| 30.8 | 2.90 | 15 |
| 31.1 | 2.87 | 16 |
| 33.6 | 2.67 | 18 |
| 34.0 | 2.64 | 27 |
| 34.4 | 2.61 | 10 |
| 34.6 | 2.59 | 10 |
| 34.8 | 2.58 | 15 |
| 35.6 | 2.52 | 17 |
| 35.7 | 2.51 | 15 |
| 36.6 | 2.45 | 6 |
| 36.9 | 2.43 | 8 |
| 38.2 | 2.35 | 11 |
| 38.7 | 2.33 | 9 |
| 38.9 | 2.32 | 13 |
| 39.1 | 2.30 | 17 |
| 39.7 | 2.27 | 17 |
| 39.9 | 2.26 | 11 |

In some embodiments, the characterization data of 2C-B Sulfate are as provided in FIG. 62 and Table 68.

TABLE 68
XRPD Signal angle data of 2C-B Sulfate

| Position (°2θ) | d-value | Relative |
|---|---|---|
| 8.7 | 10.19 | 97 |
| 13.0 | 6.79 | 60 |
| 15.1 | 5.87 | 16 |
| 16.4 | 5.39 | 26 |
| 17.4 | 5.09 | 3 |
| 17.9 | 4.94 | 35 |
| 19.6 | 4.53 | 11 |
| 19.9 | 4.47 | 15 |
| 20.4 | 4.36 | 22 |
| 20.9 | 4.24 | 22 |
| 21.3 | 4.16 | 13 |
| 21.6 | 4.11 | 17 |
| 21.8 | 4.08 | 46 |
| 22.5 | 3.96 | 4 |
| 22.7 | 3.92 | 8 |
| 23.2 | 3.83 | 66 |
| 23.8 | 3.73 | 8 |
| 24.1 | 3.69 | 5 |
| 24.8 | 3.58 | 20 |
| 25.1 | 3.55 | 100 |
| 25.4 | 3.51 | 14 |
| 25.6 | 3.47 | 65 |
| 26.2 | 3.40 | 22 |
| 26.5 | 3.36 | 10 |
| 26.8 | 3.33 | 9 |
| 27.1 | 3.30 | 8 |
| 27.6 | 3.24 | 11 |
| 27.8 | 3.21 | 24 |
| 28.0 | 3.18 | 10 |
| 28.5 | 3.13 | 34 |
| 29.0 | 3.08 | 21 |
| 29.3 | 3.05 | 10 |
| 29.7 | 3.01 | 7 |
| 31.3 | 2.85 | 5 |
| 31.9 | 2.81 | 20 |
| 32.9 | 2.73 | 10 |
| 33.1 | 2.70 | 18 |
| 33.6 | 2.67 | 6 |
| 34.1 | 2.63 | 18 |
| 34.7 | 2.59 | 7 |
| 35.0 | 2.57 | 11 |
| 35.2 | 2.55 | 13 |
| 35.8 | 2.51 | 3 |
| 36.8 | 2.44 | 15 |
| 37.2 | 2.42 | 5 |
| 37.4 | 2.41 | 7 |
| 37.5 | 2.40 | 5 |
| 37.7 | 2.38 | 8 |
| 37.8 | 2.38 | 6 |
| 38.0 | 2.37 | 5 |
| 38.7 | 2.33 | 5 |
| 39.1 | 2.30 | 5 |
| 39.7 | 2.27 | 8 |
| 39.9 | 2.26 | 7 |

In some embodiments, the characterization data of 2C-B Xinafoate are as provided in FIG. 63 and Table 69.

TABLE 69
XRPD Signal angle data of 2C-B Xinafoate

| Position (°2θ) | d-value | Relative |
|---|---|---|
| 7.9 | 11.22 | 83 |
| 8.3 | 10.65 | 11 |
| 13.1 | 6.78 | 14 |
| 13.9 | 6.39 | 35 |
| 14.4 | 6.17 | 34 |
| 14.7 | 6.02 | 21 |
| 15.1 | 5.86 | 26 |
| 15.7 | 5.65 | 10 |
| 16.5 | 5.37 | 31 |
| 17.3 | 5.13 | 19 |
| 18.6 | 4.78 | 85 |
| 19.5 | 4.56 | 13 |
| 20.1 | 4.43 | 69 |
| 20.7 | 4.30 | 15 |
| 20.9 | 4.25 | 23 |
| 21.5 | 4.14 | 26 |
| 22.5 | 3.96 | 63 |
| 23.3 | 3.81 | 87 |
| 23.6 | 3.77 | 22 |
| 24.5 | 3.64 | 30 |
| 24.8 | 3.59 | 100 |
| 24.9 | 3.57 | 70 |
| 25.4 | 3.50 | 20 |
| 26.2 | 3.40 | 63 |
| 26.7 | 3.34 | 27 |
| 27.1 | 3.29 | 19 |
| 27.5 | 3.25 | 34 |
| 27.8 | 3.21 | 30 |
| 28.1 | 3.18 | 22 |
| 28.5 | 3.13 | 15 |
| 28.9 | 3.09 | 16 |
| 29.2 | 3.06 | 16 |
| 29.4 | 3.04 | 17 |
| 29.8 | 3.00 | 19 |
| 30.4 | 2.94 | 20 |
| 31.3 | 2.86 | 19 |
| 31.6 | 2.83 | 28 |
| 32.4 | 2.76 | 21 |
| 33.1 | 2.70 | 13 |
| 33.7 | 2.66 | 14 |
| 34.2 | 2.62 | 14 |
| 34.7 | 2.58 | 22 |
| 34.9 | 2.57 | 25 |
| 35.4 | 2.54 | 13 |
| 35.9 | 2.50 | 15 |
| 36.4 | 2.47 | 11 |
| 37.0 | 2.43 | 12 |
| 37.2 | 2.42 | 15 |
| 37.7 | 2.39 | 15 |
| 37.8 | 2.38 | 16 |
| 38.7 | 2.33 | 14 |

In some embodiments, the characterization data of 2C-B Tartrate (Form 1) are as provided in FIG. 64 and Table 70.

TABLE 70
XRPD Signal angle data of 2C-B Tartrate (Form 1)

| Position (°2θ) | d-value | Relative |
|---|---|---|
| 7.2 | 12.35 | 4 |
| 10.3 | 8.60 | 20 |
| 12.9 | 6.87 | 13 |
| 14.2 | 6.22 | 34 |
| 14.4 | 6.16 | 35 |
| 15.5 | 5.73 | 11 |
| 16.6 | 5.34 | 10 |
| 17.4 | 5.08 | 11 |
| 20.3 | 4.38 | 100 |
| 20.5 | 4.32 | 32 |
| 21.4 | 4.15 | 6 |
| 21.7 | 4.10 | 13 |
| 22.3 | 3.98 | 7 |
| 23.2 | 3.83 | 29 |
| 24.0 | 3.71 | 22 |
| 24.3 | 3.67 | 16 |
| 24.7 | 3.60 | 56 |
| 25.6 | 3.48 | 16 |
| 26.1 | 3.41 | 10 |
| 27.2 | 3.27 | 46 |
| 27.5 | 3.24 | 11 |
| 28.7 | 3.11 | 12 |
| 29.0 | 3.08 | 10 |
| 29.6 | 3.02 | 12 |
| 31.7 | 2.82 | 26 |
| 32.6 | 2.75 | 8 |
| 33.3 | 2.69 | 8 |
| 33.7 | 2.66 | 22 |
| 34.2 | 2.62 | 17 |
| 34.7 | 2.59 | 11 |
| 35.9 | 2.50 | 16 |
| 37.4 | 2.40 | 8 |
| 37.8 | 2.38 | 8 |
| 38.1 | 2.36 | 8 |
| 38.5 | 2.34 | 7 |
| 39.0 | 2.31 | 11 |
| 39.6 | 2.27 | 7 |

Based on this data, Tartrate (Form 1) was selected for polymorph screening.

The majority of experiments generated Tartrate (Form 1). Only one polymorph was identified, designated as Tartrate (Form 2). It was produced from the scale-up experiment targeting Tartrate (Form 1), which was an ambient temperature slurry in EtOH. During the screen, it was observed from an elevated temperature slurry in EtOAc, a cooling experiment involving acetone, and in trace amounts as a mixture with Tartrate (Form 1) from an EtOH/iPrOAc precipitation experiment at elevated temperature. It was also produced from reaction crystallization experiments using EtOAc/MeOH and IPE/MeOH. The XRPD pattern is shown below.

In some embodiments, the characterization data of 2C-B Tartrate (Form 2) are as provided in FIG. 65 and Table 71.

TABLE 71
XRPD Signal angle data of 2C-B Tartrate (Form 2)

| Position (°20) | d-value | Relative |
|---|---|---|
| 6.7 | 13.23 | 8 |
| 11.0 | 8.07 | 56 |
| 12.0 | 7.36 | 5 |
| 12.4 | 7.15 | 28 |
| 13.3 | 6.65 | 25 |
| 13.8 | 6.44 | 7 |
| 15.6 | 5.67 | 6 |
| 16.4 | 5.42 | 10 |
| 17.1 | 5.17 | 7 |
| 17.4 | 5.10 | 9 |
| 18.0 | 4.94 | 15 |
| 18.7 | 4.75 | 6 |
| 20.6 | 4.32 | 19 |
| 20.9 | 4.25 | 64 |
| 21.2 | 4.20 | 12 |
| 22.6 | 3.93 | 100 |
| 23.0 | 3.87 | 18 |
| 23.1 | 3.85 | 18 |
| 23.4 | 3.80 | 14 |
| 24.3 | 3.66 | 19 |
| 24.5 | 3.63 | 18 |
| 24.8 | 3.59 | 25 |
| 25.6 | 3.48 | 27 |
| 26.1 | 3.41 | 15 |
| 26.8 | 3.33 | 26 |
| 27.3 | 3.26 | 13 |
| 28.4 | 3.14 | 16 |
| 28.8 | 3.10 | 37 |
| 29.4 | 3.04 | 13 |
| 31.4 | 2.85 | 13 |
| 32.5 | 2.75 | 7 |
| 33.1 | 2.70 | 10 |
| 33.8 | 2.65 | 11 |
| 34.3 | 2.61 | 12 |
| 35.3 | 2.54 | 8 |
| 36.6 | 2.45 | 13 |
| 37.1 | 2.43 | 9 |
| 37.9 | 2.37 | 9 |
| 38.9 | 2.31 | 7 |
| 39.7 | 2.27 | 8 |

Tartrate (Form 2) is crystalline and the NMR spectrum is consistent with salt formation (peak shifts observed). It has 1:1 stoichiometry and is anhydrous as there is only residual EtOH (0.1 moles) by NMR and no significant weight loss in the TG thermogram (0.2%). The DSC thermogram shows two endotherms with peak maxima at 200° C. and 202° C. These could be due to melting and may indicate Tartrate (Form 2) undergoes form conversion upon heating. Alternatively, the original material could be a mixture of forms or as a result of the material beginning to sublime.

The XRPD patterns of 2C-B Tartrate polymorphs are provided in FIG. 66.

### Background

A salt screen of 4-bromo-2,5-dimethoxyphenethylamine (2C-B) free base as well as a polymorph screen of a selected salt was performed. 2C-B is a DEA Schedule I controlled substance and a potent material.

The goal of this study was to identify and characterize crystalline salts of 2C-B. A polymorph screen was then conducted with a selected salt.

### Results and Discussion

### Salt Screening

2C-B free base was prepared from the 2C-B HCl salt (Form A). Experimental details and proton nuclear magnetic resonance spectroscopy (¹H NMR) characterization data are summarized in Table 72A and Table 72B, respectively. Note that the free base was initially obtained as poorly crystalline solids; however, it was produced as an oil at larger scale.

TABLE 72A
Preparation of 2C-B Free Base

| | XRPD | FIG. |
|---|---|---|
| Conditions^{a} | Results^{b} | Number |
| Added 15.2 mL of 1N NaOH to a solution of 3.01 g | FB1; LC | FIG. 6 |
| of 2C-B HCl in 60 mL of water (cloudy, then | | |
| oiling observed). Stirring, RT, 1 d. Extracted | | |
| w/EtOAc (3x). Combined organic layers and | | |
| dried w/MgSO₄. RE; then dry air purge (tacky | | |
| solids). VD, RT, 6 d. | | |
| Added 25.8 mL of 1N NaOH to a solution of 5.10 g | — | — |
| of 2C-B HCl in 100 mL of water (cloudy, then | | |
| oiling observed). Stirring, RT, 1 d. Extracted | | |
| w/EtOAc (3x). Combined organic layers and | | |
| dried w/MgSO₄. Evaporated w/stream of air (oil). | | |
| VD, RT, 2 mos (oil + some solids). | | |

^{a}NaOH = sodium hydroxide; EtOAc = ethyl acetate; MgSO₄ = magnesium sulfate; RE = rotary evaporation; VD = vacuum drying; RT = room/ambient temperature; d = day(s); w/ = with; mos = months
^{b}FB = 2C-B free base; LC = low crystallinity

TABLE 72B
¹H NMR Characterization of Free Base

| NMR Sample^{a} | Results^{b} | FIG. Number |
|---|---|---|
| Sample | Consistent with structure | 49 |
| (DMSO-d₆) | Peak shifting observed relative to | |
| | 2C-B HCl spectrum | |
| | 0.06 moles MeOH^{c} | |
| | Unidentified peaks at 3.1 and 6.7 ppm | |

^{a}DMSO-d₆ = deuterated dimethyl sulfoxide
^{b}MeOH = methanol
^{c}This is suspected to be residual solvent from cleaning the NMR tube prior to analysis, and are not in the actual sample.

A salt screen was conducted with 2C-B free base and twenty-one acids. The acids were selected because each has 1) Stahl (Handbook of Pharmaceutical Salts; Stahl, P.; Wermuth, C. G., Eds.; VHCA and Wiley-VCH: Zurich and Weinheim, 2008) class 1 or 2 status and 2) a pKₐ value that is at least 2-3 units below the pKₐ of 2C-B (expected to be between 10 and 11). Experiments used one molar equivalent of acid and are described in Table 18. Designations of “new 1, new 2, etc.” were used if salt formation and presence of counterion were not confirmed by ¹H NMR.

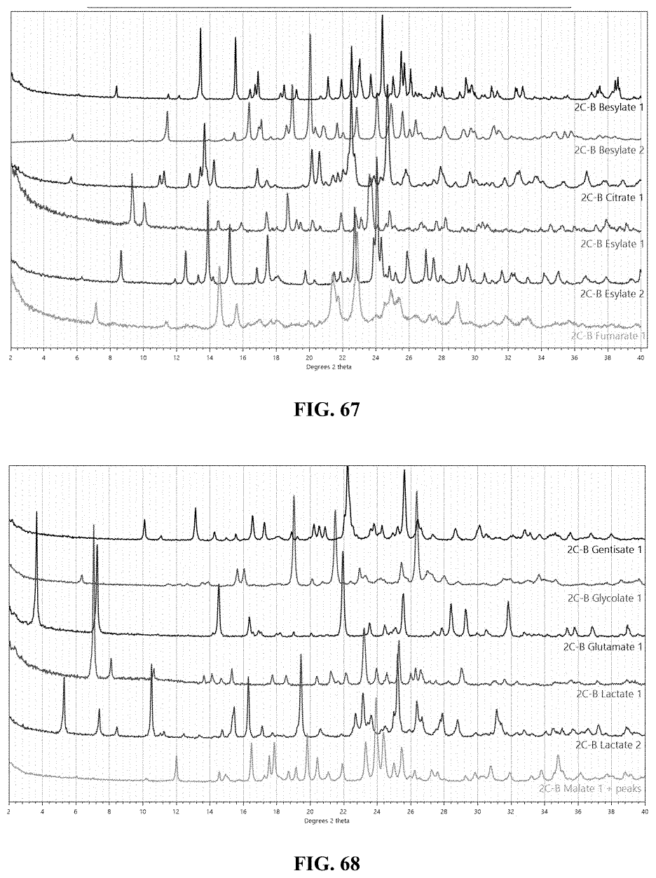

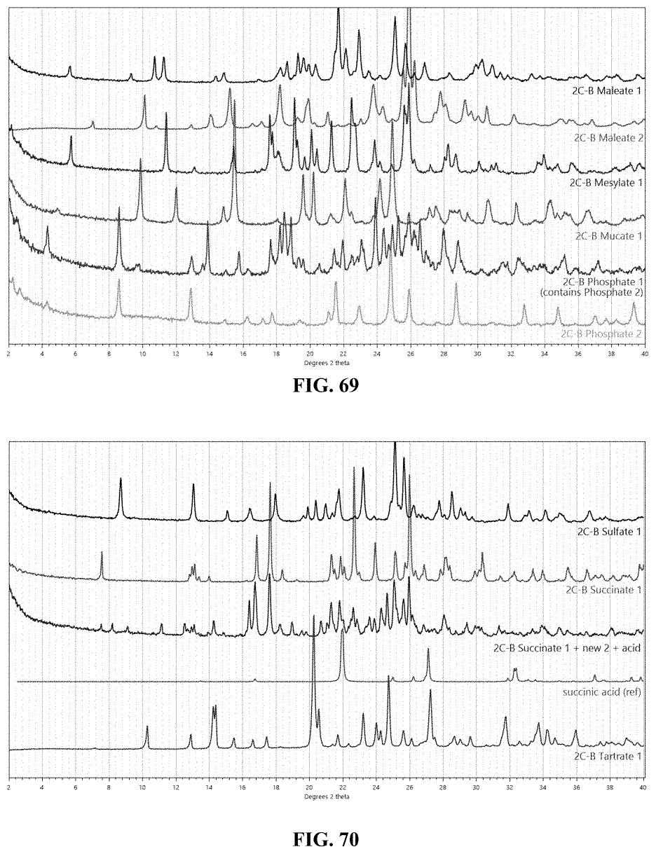

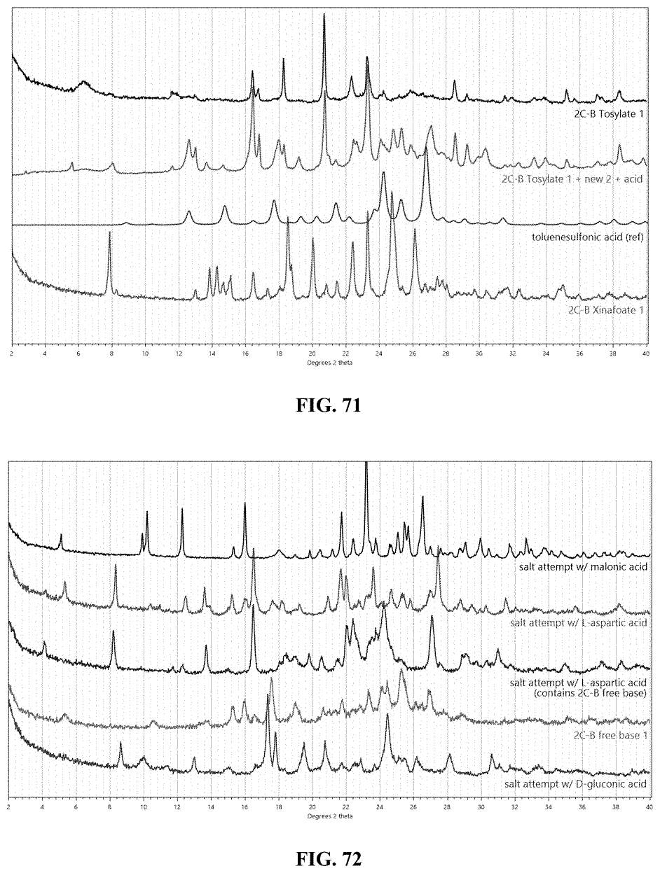

Crystalline salts were generated with 2C-B free base and the following acids: benzenesulfonic acid, citric acid, ethanesulfonic acid, fumaric acid, gentisic acid, glutamic acid, glycolic acid, D,L-lactic acid, L-malic acid, maleic acid, methanesulfonic acid, mucic (galactaric) acid, phosphoric acid, succinic acid, sulfuric acid, L-tartaric acid, toluenesulfonic acid, and 1-hydroxy-2-naphthoic (xinafoic) acid. The XRPD patterns are shown below in FIG. 67 through 71.

It should be mentioned that a potential salt was generated with L-aspartic acid (FIG. 72). However, a consistent XRPD pattern was not obtained and because phase purity was uncertain, the material was not further characterized. Salt formation could not be confirmed.

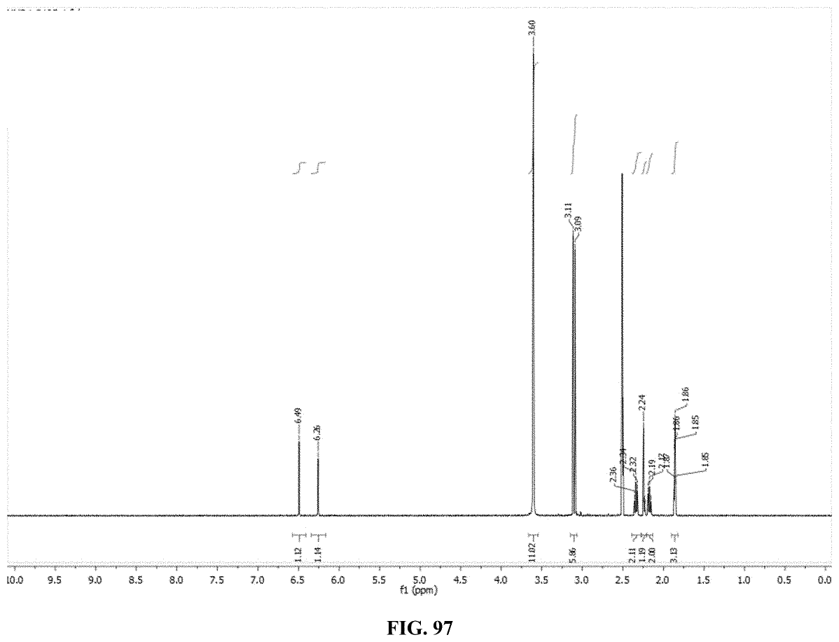

A crystalline material with 1:5.5 (API:acid) stoichiometry by NMR was also produced with malonic acid (FIG. 97). Based on the stoichiometry, formation of a stoichiometric salt is unlikely, but further study would be needed to confirm the nature of this material.

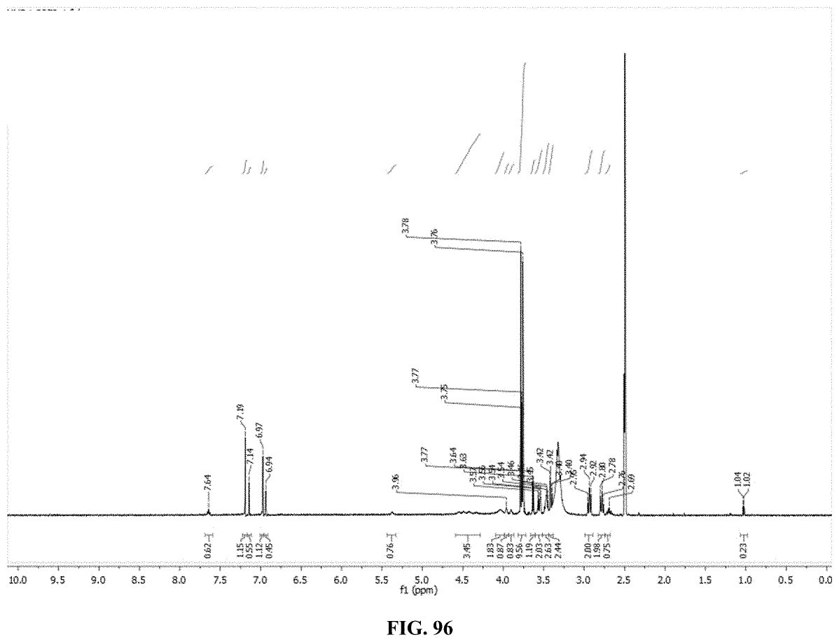

A unique, but poorly crystalline material was observed from experiments with D-gluconic acid. Based on NMR analysis, it was composed of an unknown degradant or impurity, indicating salt formation did not occur (FIG. 96).

Crystalline materials appearing to be composed of a single phase by XRPD were characterized by ¹H NMR and, for inorganic counter-ions, ion chromatography (IC). Data is summarized in Table 74.

TABLE 74
1H NMR and IC Characterization of Salts

| | Analytical | | |
|---|---|---|---|
| Sample | Technique^{a} | Results^{b} | FIG. |
| Besylate | ¹H NMR | Consistent with structure | FIG. 73 |
| (Form 1) | (DMSO-d₆) | 1:1 stoichiometry | |
| | | No organic solvents | |
| Besylate | ¹H NMR | Consistent with structure | FIG. 74 |
| (Form 2) | (DMSO-d₆) | 1:1 stoichiometry | |
| | | 0.03 moles MEK | |
| Citrate | ¹H NMR | Consistent with structure | FIG. 75 |
| (Form 1) | (d-MeOD) | 1:1 stoichiometry | |
| | | 0.05 moles MEK^{c} | |
| Esylate 1 | ¹H NMR | Consistent with structure | FIG. 76 |
| | (DMSO-d₆) | 1:1 stoichiometry | |
| | | No organic solvents | |
| Esylate 2 | ¹H NMR | Consistent with structure | FIG. 77 |
| | (d-MeOD) | 1:1 stoichiometry | |
| | | No organic solvents^{c} | |
| Fumarate | ¹H NMR | Consistent with structure | FIG. 78 |
| (Form 1) | (d-MeOD) | Likely 2:1 API:acid | |
| | | stoichiometry (contains slight | |
| | | excess of acid) | |
| | | No organic solvents | |
| Gentisate | ¹H NMR | Consistent with structure | FIG. 79 |
| | (DMSO-d₆) | 1:1 stoichiometry | |
| | | 0.1 moles ACN | |
| | | Minor peak at 1.1 ppm | |
| Glutamate | ¹H NMR | Consistent with structure | FIG. 80 |
| | (DMSO-d₆) | 1:1 stoichiometry | |
| | | No organic solvents | |
| Glycolate | ¹H NMR | Consistent with structure | FIG. 81 |
| | (DMSO-d₆) | 1:1 stoichiometry | |
| | | 0.1 moles MTBE | |
| Lactate | ¹H NMR | Consistent with structure | FIG. 82 |
| (Form 1) | (DMSO-d₆) | 1:0.8 API:acid stoichiometry | |
| | | 0.8 moles IPA | |
| | | Peaks at 1.3, 4.1, and 4.7 ppm are | |
| | | suspected impurities in _{D, L}-lactic | |
| | | acid | |
| Lactate | ¹H NMR | Consistent with structure | FIG. 83 |
| (Form 2) | (DMSO-d₆) | 1:0.8 API:acid stoichiometry | |
| | | 0.5 moles iPrOAc | |
| | | Peaks at 1.3, 4.1, and 4.7 ppm are | |
| | | suspected impurities in _{D, L}-lactic | |
| | | acid | |
| Malate + | ¹H NMR | Consistent with structure | FIG. 84 |
| peaks | (DMSO-d₆) | 1:1 API:acid stoichiometry | |
| | | 0.01 moles MEK | |

^{a}DMSO-d₆ = deuterated dimethyl sulfoxide; d-MeOD = deuterated
methanol
^{b}API = Active Pharmaceutical Ingredient; MTBE = methyl tert-butyl
ether; IPA = isopropyl alcohol; iPrOAc = isopropyl acetate; ACN =
acetonitrile.
^{c}Note the peaks at 2.16 and/or 2.67 ppm are attributable to acetone and
DMSO, respectively. These are suspected to be residual solvents from
cleaning the NMR tube prior to analysis, and are not in the actual sample.

| | Analytical | | |
|---|---|---|---|
| Sample | Technique^{a} | Results^{b} | FIG. |
| Maleate | ¹H NMR | Consistent with structure | FIG. 85 |
| (Form 1) | (DMSO-d₆/ | 1:1 stoichiometry | |
| | d-MeOD) | No organic solvents | |
| Maleate | ¹H NMR | Consistent with structure | FIG. 86 |
| (Form 2) | (DMSO-d₆) | 1:1 stoichiometry | |
| | | 0.8 moles MEK | |
| Mesylate | ¹H NMR | Consistent with structure | FIG. 87 |
| | (DMSO-d₆) | 1:1 stoichiometry | |
| | | No organic solvents | |
| Mucate | ¹H NMR | Consistent with structure | FIG. 88 |
| | (DMSO-d₆/ | 1:0.75 API:acid stoichiometry | |
| | d-MeOD) | No organic solvents | |
| Phosphate | ¹H NMR | Consistent with structure | FIG. 89 |
| (Form 1) | (d-MeOD) | No organic solvents | |
| | IC | Theoretical (1:1, hemi-hydrate): | |
| | | 25.9% PO₄⁻³ | |
| | | Reported: 26.1% PO₄⁻³ | |
| Phosphate | ¹H NMR | Consistent with structure | FIG. 90 |
| (Form 2) | (DMSO-d₆) | 2 moles IPA | |
| | IC | Theoretical (1:1, w/2 moles | |
| | | IPA): 19.8% PO₄⁻³ | |
| | | Reported: 25.0% PO₄⁻³ | |
| Succinate | ¹H NMR | Consistent with structure | FIG. 91 |
| (Form 1) | (DMSO-d₆) | 2:1 API:acid stoichiometry | |
| | | No organic solvents | |
| Sulfate | ¹H NMR | Consistent with structure | FIG. 92 |
| | (DMSO-d₆) | 0.02 moles IPA | |
| | | 0.01 moles acetone | |
| Tartrate | ¹H NMR | Consistent with structure | FIG. 93 |
| (Form 1) | (d-MeOD) | 1:1 stoichiometry | |
| | | No organic solvents^{c} | |
| Tosylate | ¹H NMR | Consistent with structure | FIG. 94 |
| (Form 1) | (DMSO-d₆) | 1:1 stoichiometry | |
| | | No organic solvents | |
| Xinafoate | ¹H NMR | Consistent with structure | FIG. 95 |
| | (DMSO-d₆) | 1:1 API:acid stoichiometry | |
| | | 0.1 moles iPrOAc | |
| salt attempt | ¹H NMR | Inconsistent with structure- | FIG. 96 |
| with D- | (DMSO-d₆) | additional peaks present | |
| gluconic acid | | throughout spectrum | |
| salt attempt | ¹H NMR | Consistent with structure | FIG. 97 |
| with malonic | (DMSO-d₆) | 1:5.5 API:acid stoichiometry | |
| acid | | No organic solvents | |
| | | Unidentified peaks at 1.9 and | |
| | | 2.2 ppm | |

^{a}DMSO-d₆ = deuterated dimethyl sulfoxide; d-MeOD = deuterated
methanol
^{b}API = Active Pharmaceutical Ingredient; MTBE = methyl tert-butyl
ether; IPA = isopropyl alcohol; iPrOAc = isopropyl acetate; ACN =
acetonitrile; MEK = methyl ethyl ketone.
^{c}Note the peaks at 2.16 and/or 2.67 ppm are attributable to acetone and
DMSO, respectively. These are suspected to be residual solvents from
cleaning the NMR tube prior to analysis, and are not in the actual sample.

Salts that did not contain organic solvents by NMR were further characterized by thermogravimetric analysis (TGA) and differential scanning calorimetry (DSC). Data is summarized in Table 75.

TABLE 75
Thermal Characterization of Salts

| | Analytical | | |
|---|---|---|---|
| Sample | Technique | Results^{a} | FIG. |
| Besylate | TGA | 0.7%, start to 199° C. | FIG. 107 |
| (Form 1) | DSC | Endo: 160° C., 203° C. | |
| | | (onset 202° C., | |
| | | Δ H°_{fus} = 105.9 J/g) | |
| Besylate | TGA | 2.9%, start to 217° C. | FIG. 108 |
| (Form 2) | DSC | Endo: 201° C. (onset 198° C.) | |
| Citrate | TGA | 0.1%, start to 126° C. | |
| (Form 1) | DSC | Endo: 119° C. (onset 115° C., | FIG. 109 |
| | | ΔH°_{fus} = 77.6 J/g) | |
| Esylate 1 | TGA | 0.2%, start to 195° C. | |
| | DSC | Endo: 54° C., 162° C. | FIG. 110 |
| | | (onset 161° C., | |
| | | ΔH°_{fus} = 81.5 J/g) | |
| Esylate 2 | TGA | 0.3%, start to 198° C. | |
| | DSC | Endo: 59° C., 162° C. | FIG. 111 |
| | | (onset 161° C., | |
| | | ΔH°_{fus} = 71.1 J/g) | |
| | | Exo: 135° C. | |
| Fumarate | TGA | 0.3%, start to 158° C. | FIG. 112 |
| (Form 1) | DSC | Endo: 191° C. | |
| Gentisate | TGA | 1.2%, start to 178° C. | FIG. 113 |
| | DSC | Endo: 124° C., 215° C. | |
| Glutamate | TGA | 5.3%, start to 143° C. | FIG. 114 |
| | DSC | Endo: 77° C., 92° C., 182° C. | |
| Glycolate | TGA | 0.4%, start to 131° C. | FIG. 115 |
| | DSC | Endo: 151° C. (onset 149° C., | |
| | | ΔH°_{fus} = 131.4 J/g) | |
| Malate + | TGA | 0.1%, start to 146° C. | FIG. 116 |
| peaks | DSC | Endo: 151° C. (onset 149° C., | |
| | | ΔH°_{fus} = 85.7 J/g) | |
| Maleate | TGA | 0.5%, start to 153° C. | FIG. 117 |
| (Form 1) | DSC | Endo: 166° C. | |
| Mesylate | TGA | No weight loss prior to 200° C. | FIG. 118 |
| | DSC | Endo: 182° C. (onset 181° C., | |
| | | ΔH°_{fus} = 89.3 J/g) | |
| Phosphate | TGA | 2.7%, start to 98° C. | FIG. 119 |
| (Form 1) | | 17.9%, 98° C. to 195° C. | |
| | DSC | Endo: 107° C., 183° C., 219° C. | |
| Succinate | TGA | 0.2%, start to 150° C. | FIG. 120 |
| (Form 1) | DSC | Endo: 188° C., 193° C. | |
| Sulfate | TGA | 0.2%, start to 156° C. | FIG. 121 |
| | DSC | Endo: 57° C., 169° C. | |
| | | (onset 167° C., | |
| | | ΔH°_{fus} = 87.7 J/g) | |
| Tartrate | TGA | No weight loss prior to 184° C. | FIG. 122 |
| (Form 1) | DSC | Endo: 206° C. | |
| Tosylate | TGA | 0.3%, start to 204° C. | FIG. 123 |
| (Form 1) | DSC | Endo: 154° C., 197° C. | |
| | | (onset 196° C., | |
| | | ΔH°_{fus} = 70.0 J/g) | |
| Xinafoate | TGA | 0.4%, start to 120° C. | FIG. 124 |
| | | 1.3%, 120° C. to 162° C. | |
| | DSC | Endo: 184° C. | |

^{a}TGA = thermogravimetric analysis; DSC = differential scanning calorimetry; Endo = endotherm; Exo = exotherm; ΔH°_{fus} = heat of fusion

Several salts were selected for scale-up and further characterization. Selection was based on crystallinity, stoichiometry, solvation state, thermal behavior, and reproducibility. Crystalline, stoichiometric salts that were anhydrous, had high melting points (greater than ˜120° C.), and where only one form had been observed in screening were prepared at approximately 0.5 gram scale. Experimental details and results are summarized in Table 76A.

TABLE 76A
Scale-up and Characterization of Selected Salts

| Targeted | | | FIG. |
|---|---|---|---|
| Salt | Conditions^{a} | XRPD Results^{b} | No. |
| Besylate | P, EtOH, RT | Besylate (Form 1) | 55 |
| (Form 1) | | | |
| Citrate | P, MEK, 50° C. | — | — |
| (Form 1) | (NS)−>RT; gel. | | |
| | SL, EtOAc, 50° C., | Citrate (Form 2) | 39 |
| | 1 d−>RT, 4 d | | |
| Fumarate | P, ACN, 50° C.−>RT | Fumarate (Form 1) + | |
| (Form 1) | | Fumarate (Form 2) + | |
| | | acid; LC | |
| | SL, IPE, 50° C., | Fumarate (Form 1) + | |
| | 1 d−>RT, 5 d | acid + pks; LC | |
| Gentisate | SL, ACN, 50° C., | Gentisate | 57 |
| | 2 d−>RT, 3 d | | |
| Glycolate | SL, MTBE, 50° C., | Glycolate | |
| | 1 d−>RT, 4 d | | |
| Malate | P, MEK, 50° C.−>RT | Malate; indexed | 60 |
| Mesylate | P, ACN, 50° C.−>RT | Mesylate | 61 |
| Sulfate | P, IPA, RT | Sulfate | |
| Tartrate | SL, EtOH, RT, 5 d | Tartrate (Form 1) | |
| (Form 1) | | | |

^{a}EtOH = ethanol; MEK = methyl ethyl ketone; EtOAc = ethyl acetate; ACN = acetonitrile; IPE = di-isopropyl ether; MTBE = methyl tert-butyl ether; IPA = isopropanol; P = precipitation; SL = slurry; RT = room/ambient temperature; NS = no solids; d = day(s)
^{b}LC = low crystallinity; pk(s) = peak(s)

The targeted form for the besylate, gentisate, glycolate, malate, mesylate, sulfate, and tartrate salts were successfully generated, while a new form of the citrate salt, Citrate (Form 2), was obtained from experiments targeting Citrate (Form 1). The fumarate salt was only obtained as a mixture and was therefore not further pursued.

The besylate, citrate, gentisate, glycolate, malate, mesylate, sulfate, and tartrate salts were characterized by XRFD, ¹H NMR (or IC), TGA, DSC, dynamic vapor sorption (DVS) with XRPD of the post-DVS sample, optical microscopy (OM), scanning electron microscopy (SEM), and energy dispersive X-ray spectroscopy (EDX), if applicable. Characterization data is provided in Table 76B.

TABLE 76B
Characterization of Salts

| | Analytical | | | |
|---|---|---|---|---|
| Salt | Technique^{a} | Analysis | Results^{b} | FIG. |
| Besylate | (DMSO-d₆) | (DMSO-d₆) | Consistent with structure | FIG. 98 |
| (Form 1) | | | 1:1 stoichiometry | |
| | | | 0.01 moles EtOH | |
| | TGA | — | 0.3%, start to 218° C. | FIG. 125 |
| | DSC | — | Endo: 163° C., 203° C. | |
| | | | (onset 202° C., | |
| | | | ΔH°_{fus} = 103.4 J/g) | |
| | DVS | — | Sorption: | FIG. 137 |
| | | | 5-95% RH: 0.7% gain | |
| | | | Desorption: | |
| | | | 95-5% RH: 0.7% loss | |
| | post-DVS | | Consistent with Besylate (Form 1) | |
| | XRPD | | | |
| | OM | — | Rods and plates | |
| | | | Birefringence observed | |
| Citrate | ¹HNMR | (d-MeOD) | Consistent with structure | FIG. 99 |
| (Form 2) | | | 2:1 (API:acid) stoichiometry | |
| | | | No organic solvents | |
| | TGA | — | 0.2%, start to 124° C. | FIG. 126 |
| | DSC | — | Endo: 176° C. | |
| | DVS | — | Sorption: | FIG. 138 |
| | | | 5-95% RH: 0.5% gain | |
| | | | Desorption: | |
| | | | 95-5% RH: 0.5% loss | |
| | post-DVS | | Consistent with Citrate (Form 2) | |
| | XRPD | | | |
| | OM | — | Rods and plates | |
| | | | Birefringence observed | |

^{a}d-MeOD = deuterated methanol; DMSO-d₆ = deuterated dimethyl sulfoxide
^{b}API = active pharmaceutical ingredient; endo = endotherm; RH = relative humidity;
ΔH°_{fus} = heat of fusion; EtOH = ethanol

| | Analytical | | | |
|---|---|---|---|---|
| Salt | Technique^{a} | Analysis | Results^{b} | Page |
| Gentisate | ¹H NMR | (DMSO-d₆) | Consistent with structure | FIG. 100 |
| | | | 1:1 stoichiometry | |
| | | | 0.06 moles ACN | |
| | TGA | — | 0.3%, start to 184° C. | FIG. 127 |
| | DSC | — | Endo: 124° C., 214° C. | |
| | | | (onset 213° C., | |
| | | | ΔH°_{fus} = 109.8 J/g) | |
| | DVS | — | Sorption: | FIG. 139 |
| | | | 5-95% RH: 0.2% gain | |
| | | | Desorption: | |
| | | | 95-5% RH: 0.2% loss | |
| | post-DVS | | Crystalline, consistent | |
| | XRPD | | with Gentisate | |
| | OM | — | Rods + plates | |
| | | | Birefringence observed | |
| Glycolate | ¹HNMR | (DMSO-d₆) | Consistent with structure | FIG. 101 |
| | | | 1:1 stoichiometry | |
| | | | Peak shifts consistent | |
| | | | with salt formation | |
| | | | 0.01 moles MTBE | |
| | TGA | — | 0.9%, start to 125° C. | FIG. 128 |
| | DSC | — | Endo: 149° C., 161° C. | |
| | | | Exo: 151° C. | |
| | DVS | — | Sorption: | FIG. 140 |
| | | | 5-85% RH: 0.9% gain | |
| | | | 85-95% RH: 2.7% gain | |
| | | | Total gain: 3.6% | |
| | | | Desorption: | |
| | | | 95-85% RH: 2.6% loss | |
| | | | 85-5% RH: 1.1% loss | |
| | | | Total loss: 3.7% | |
| | post-DVS | | Consistent with Glycolate | |
| | XRPD | | | |
| | OM | — | Unknown morphology, aggregates | |
| | | | Birefringence observed | |

^{a}d-MeOD = deuterated methanol; DMSO-d₆ = deuterated dimethyl sulfoxide
^{b}API = active pharmaceutical ingredient; endo = endotherm; RH = relative humidity;
ΔH°_{fus} = heat of fusion; ACN = acetonitrile; MTBE = methyl tert-butyl ether

| | Analytical | | | |
|---|---|---|---|---|
| Salt | Technique^{a} | Analysis | Results^{b} | FIG. |
| Malate | ¹H NMR | (DMSO-d₆) | Consistent with structure | FIG. 102 |
| | | | 1:1 stoichiometry | |
| | | | 0.01 moles MEK | |
| | TGA | — | 0.3%, start to 150° C. | FIG. 129 |
| | DSC | — | Endo: 152° C. | |
| | DVS | — | Sorption: | FIG. 141 |
| | | | 5-85% RH: 0.6% gain | |
| | | | 85-95% RH: 1.0% gain | |
| | | | Total gain: 1.6% | |
| | | | Desorption: | |
| | | | 95-85% RH: 1.0% loss | |
| | | | 85-5% RH: 0.6% loss | |
| | | | Total loss: 1.6% | |
| | post-DVS | | Consistent with Malate | |
| | XRPD | | | |
| | OM | — | Irregular, unknown | |
| | | | morphology; aggregates | |
| | | | Birefringence observed | |
| Mesylate | ¹H NMR | (DMSO-d₆) | Consistent with structure | FIG. 103 |
| | | | 1:1 stoichiometry | |
| | | | No organic solvents | |
| | TGA | — | 0.8%, start to 200° C. | FIG. 130 |
| | DSC | — | Endo: 179° C. | |
| | DVS | — | Sorption: | FIG. 142 |
| | | | 5-85% RH: 2.7% gain | |
| | | | 85-95% RH: 4.5% gain | |
| | | | Total gain: 7.2% | |
| | | | Desorption: | |
| | | | 95-85% RH: 4.4% loss | |
| | | | 85-5% RH: 3.0% loss | |
| | | | Total loss: 7.4% | |
| | post-DVS | | Consistent with Mesylate | |
| | XRPD | | | |
| | OM | — | Irregular, unknown | |
| | | | morphology; aggregates | |
| | | | Birefringence observed | |

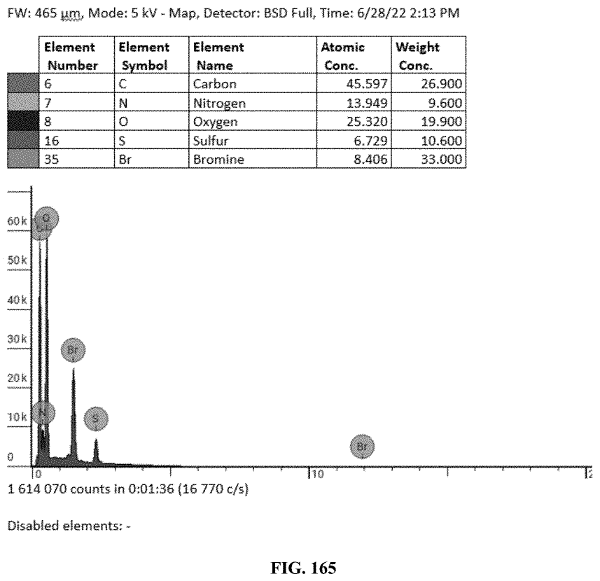

^{a}d-MeOD = deuterated methanol; DMSO-d₆ = deuterated dimethyl sulfoxide
^{b}API = active pharmaceutical ingredient; endo = endotherm; RH = relative humidity;
ΔH°_{fus} = heat of fusion; MEK = methyl ethyl ketone

| | Analytical | | | |
|---|---|---|---|---|
| Salt | Technique^{a} | Analysis | Results^{b} | FIG. |
| Sulfate | ¹H NMR | (DMSO-d₆) | Consistent with structure | FIG. 104 |
| | | | No organic solvents | |
| | EDX | | Bromine:Sulfur ratio= 1:0.8 | FIG. 165 |
| | IC | | Theoretical (1:1): 26.8% SO₄⁻² | — |
| | | | Theoretical (2:3): 35.4% SO₄⁻² | |
| | | | Reported: 36.1% SO₄⁻² | |
| | TGA | — | 0.2%, start to 160° C. | FIG. 131 |
| | DSC | — | Endo: 169° C. | |
| | | | (onset 167° C., | |
| | | | ΔH°_{fus} =119.2 J/g) | |
| | DVS | — | Sorption: | FIG. 143 |
| | | | 5-85% RH: 0.8% gain | |
| | | | 85-95% RH: 1.4% gain | |
| | | | Total gain: 2.2% | |
| | | | Desorption: | |
| | | | 95-85% RH: 1.3% loss | |
| | | | 85-5% RH: 0.8% loss | |
| | | | Total loss: 2.1% | |
| | post-DVS | | Consistent with Sulfate | |
| | XRPD | | | |
| | OM | — | Irregular, unknown | |
| | | | morphology; flake-like | |
| | | | particles | |
| | | | Birefringence observed | |
| Tartrate | ¹H NMR | (DMSO-d₆) | Consistent with structure | FIG. 105 |
| (Form 1) | | | 1:1 stoichiometry | |
| | | | No organic solvents | |
| | TGA | — | 0.1%, start to 182° C. | FIG. 132 |
| | DSC | — | Endo: 206° C. | |
| | DVS | — | Sorption: | FIG. 144 |
| | | | 5-95% RH: 0.1% gain | |
| | | | Desorption: | |
| | | | 95-5% RH: 0.1% loss | |
| | post-DVS | 1166-91-1 | Crystalline, consistent | |
| | XRPD | | with Tartrate (Form 1) | |
| | OM | — | Irregular morphology; | |
| | | | some rods + plates | |
| | | | Birefringence observed | |
| | SEM | — | — | |

^{a}DMSO-d₆ = deuterated dimethyl sulfoxide
^{b}endo = endotherm; RH = relative humidity; ΔH°_{fus} = heat of fusion

Solubilities in water were estimated for the majority of the salts identified during screening. The experiments were carried out by adding water in aliquots to weighed portions of solid. Whether dissolution had occurred was judged by visual inspection after addition of each aliquot. The results are shown in Table 77. Solubility numbers were calculated by dividing the weight of the sample by the total amount of water used to dissolve the sample. The actual solubilities may be greater than the numbers calculated because of the use of aliquots that were too large or because of slow dissolution rates. Values are reported as “≥” if complete dissolution was observed upon the first aliquot. Values are reported as “<” if dissolution did not occur during the experiment. All solubility measurements were carried out at room temperature.

Table 77 provides the estimated kinetic aqueous solubility of the salts.

TABLE 77
Estimated Kinetic Aqueous Solubility of Salts

| | Result | |
|---|---|---|
| Salt | (mg/mL) | Comments |
| Besylate | 5 | — |
| (Form 1) | | |
| Besylate | 4* | at 4 mg/mL a few, small particulates |
| (Form 2) | | remaining, floating on top; dissolved at 2 |
| | | mg/mL |
| Citrate | 10 | — |
| (Form 2) | | |
| Esylate 1 | ≥9 | — |
| Esylate 2 | 6* | at 6 mg/mL a few, small particulates |
| | | remaining, floating on top; remained at 1 |
| | | mg/mL |
| Fumarate | 8 | — |
| (Form 1) | | |
| Gentisate | 2 | — |
| Glycolate | ≥45 | — |
| Malate | 6 | — |
| Maleate | 7* | at 4 mg/mL small, translucent flakes |
| (Form 1) | | remaining, floating on top; remained at 1 |
| | | mg/mL |
| Mesylate | ≥38 | — |
| Phosphate | 2 | — |
| (Form 1) | | |
| Succinate | 14 | — |
| (Form 1) | | |
| Sulfate | 14 | — |
| Tartrate | 11 | — |
| (Form 1) | | |
| Tosylate | 5* | at 5 mg/mL small, translucent flakes |
| (Form 1) | | remaining, floating on top; clear at 2 |
| | | mg/mL |
| Xinafoate | <2 | — |

### Besylate (Form 1)

Two crystalline forms of the besylate salt were observed during screening. Besylate (Form 1) was obtained from ethanol and was successfully reproduced at larger scale. It has 1:1 stoichiometry and is anhydrous as the TG thermogram only shows a 0.3% loss from ambient to 218° C. (FIG. 107). Because there are no organic solvents by NMR, this is likely due to a minor amount of residual water and would be equivalent to less than 0.1 moles. The sharp endotherm at 203° C. in the DSC thermogram is likely due to melting. Note there is also a small endotherm at 163° C., the nature of which was not identified. While it could be due to a small impurity or form conversion upon heating, further investigation would be needed to confirm.

Based on DVS data, B.esylate (Form 1) is slightly hygroscopic and gains 0.7% moisture, equivalent to 0.2 moles of water (FIG. 137). Upon the desorption cycle, it loses all the gained moisture and does not undergo form conversion.

Deliquescence was not observed after several days at 75% RH at ambient temperature. The estimated kinetic solubility of B.esylate (Form 1) in water was visually determined to be 5 mg/mL.

Besylate (Form 2) was obtained from methyl ethyl ketone. It has 1:1 stoichiometry and may be hydrated as the TG thermogram shows a 2.9% weight loss from ambient to 217° C. (FIG. 108). Because there are no organic solvents in the NMR spectrum, this is likely due to water and would be equivalent to 0.7 moles. The sharp endotherm at 201° C. in the DSC thermogram is suspected to be due to melting. Note that the weight loss occurs concurrently with the melt, which is more typical of a hydrated material than that of an anhydrate containing residual volatiles.

The estimated kinetic solubility of B.esylate (Form 2) in water was visually determined to be 4 mg/mL. Note that, at this concentration, a few small particulates remained.

### Citrate (Form 2)

Two crystalline forms of the citrate salt were identified during screening. Citrate (Form 1) was produced from a cooling experiment with methyl ethyl ketone and from an elevated temperature slurry using ethyl acetate. It has 1:1 stoichiometry and is anhydrous as the TG thermogram only shows a 0.1% weight loss (FIG. 109). The endotherm at 119° C. in the DSC thermogram is likely due to melting.

Citrate (Form 2) was produced from a scale-up experiment targeting Citrate (Form 1) (elevated temperature slurry in ethyl acetate). Note that solids were not obtained from methyl ethyl ketone when that experiment was conducted at larger scale. Citrate (Form 2) has 2:1 2C-B:citric acid stoichiometry and is anhydrous as the TG thermogram only shows a 0.2% weight loss from ambient to 124° C. (FIG. 126). Because there are no organic solvents by NMR, the weight loss is likely due to a small amount of residual water and would be equivalent to less than 0.1 moles. The endotherm at 176° C. in the DSC thermogram is typical of melting.

Based on DVS data, Citrate (Form 2) is slightly hygroscopic and gains 0.5% moisture, equivalent to 0.1 moles of water (FIG. 138). Upon desorption, all the gained moisture is lost and form change does not occur.

Deliquescence was not observed after several days at 75% RH at ambient temperature. The estimated kinetic solubility of Citrate (Form 2) in water was visually determined to be 10 mg/mL.

### Gentisate

Only one crystalline form of the gentisate salt was observed during screening. Gentisate was produced from an elevated temperature slurry in acetonitrile and was successfully prepared at larger scale. It has 1:1 stoichiometry. The TG thermogram shows a 0.3% weight loss from ambient to 184° C., indicating that Gentisate is anhydrous (FIG. 127). Because there were trace amounts of acetonitrile in the NMR spectrum (0.07 moles), the weight loss could be due to a partial loss of acetonitrile, which would account for 0.03 moles. Alternatively, the loss could be due to residual water and would be equivalent to less than 0.1 moles.

The sharp endotherm at 214° C. in the DSC thermogram is typical of melting. Note that there is also a small shoulder at 206° C. as well as a small endotherm at 124° C. (FIG. 127). These may be attributable to the loss of residual volatiles, but could also be due to a small impurity or form conversion upon heating. Further investigation such as hotstage micrsocopy or VT-XRPD would be needed to confirm the nature of these events.

Based on DVS data, Gentisate is non-hygroscopic and only gains 0.2% moisture, equivalent to just 0.05 moles of water (FIG. 139). Upon desorption, the 0.2% moisture is lost and form change does not occur.

Deliquescence was not observed after several days at 75% RH at ambient temperature. The estimated kinetic solubility of Gentisate in water was visually determined to be 2 mg/mLError! Reference source not found.

### Glycolate

Only one crystalline form of the glycolate salt was observed during screening. Glycolate was obtained from an elevated temperature slurry in methyl tert-butyl ether and was successfully prepared at larger scale. It has 1:1 stoichiometry. The TG thermogram shows a 0.9% weight loss from ambient to 125° C., suggesting that Glycolate is anhydrous. Because there were only 0.01 moles of methyl tert-butyl ether in the NMR spectrum (which accounts for 0.3% of the loss), the remaining is likely attributable to residual water and would be equivalent to 0.1 moles. The DSC thermogram shows two endotherms at 149° C. and 161° C. There is also an exotherm at 151° C. (FIG. 128). These events suggest that, upon melting at 149° C., Glycolate recrystallizes to a different form, which subsequently melts at 161° C. Hot-stage microscopy or VT-XRPD would be needed to confirm.

Based on DVS data, Glycolate is moderately hygroscopic and gains 3.6% moisture, equivalent to 0.7 moles of water. The majority of this (2.7%) occurs above 85% RH (FIG. 140). Upon desorption, all the gained moisture is lost and form change is not apparent by XRPD.

Deliquescence was not observed after several days at 75% RH at ambient temperature. The estimated kinetic solubility of Glycolate in water was visually determined to be ≥45 mg/mL.

### Malate

Only one form of the malate salt was observed during screening. Malate was obtained from a cooling experiment using methyl ethyl ketone and was successfully reproduced at larger scale. Malate is crystalline and has 1:1 stoichiometry by NMR. The XRPD pattern of the sample made at larger scale was successfully indexed, which provides strong evidence that Malate is composed of a single crystalline phase (FIG. 145A). The unit cell volume, calculated from the indexing solution, is consistent with NMR data and indicates a 1:1 stoichiometry.

It should be mentioned that the original screening samples of Malate contain additional peaks by XRPD. These are not allowed, per the indexing solution, and could be due to a potential polymorph. They are highlighted in FIG. 145B.

Malate is believed to be anhydrous as the TG thermogram only shows a 0.3% weight loss from ambient to 150° C. (FIG. 129). The majority of the weight loss (0.2%) is likely attributable to the trace amounts of methyl ethyl ketone present by NMR (0.01 moles). Melting is suspected to occur at 152° C.

Based on DVS data, Malate is slightly hygroscopic and gains 1.6% moisture, equivalent to approximately 0.4 moles of water (FIG. 141). Upon desorption, all the gained moisture is lost without form change.

Deliquescence was not observed after several days at 75% RH at ambient temperature. The estimated kinetic solubility of Malate in water was visually determined to be 6 mg/mL.

### Mesylate

Only one crystalline form of the mesylate salt was observed during screening. Mesylate was obtained from a cooling experiment using acetonitrile and was successfully prepared at larger scale. It has 1:1 stoichiometry. The TG thermogram shows a 0.8% weight loss from ambient to 200° C., which suggests that Mesylate is anhydrous (FIG. 130). Because there are no organic solvents present by NMR, the weight loss is likely due to residual water and would be equivalent to 0.2 moles. The endotherm at 179° C. in the DSC thermogram is typical of melting.

Based on DVS data, Mesylate is hygroscopic and gains 7.2% moisture through 95% RH (FIG. 142). This would be equivalent to approximately 1.5 moles of water. Upon desorption, however, all the gained moisture is lost and form change is not apparent by XRPD.

Deliquescence was not observed after several days at 75% RH at ambient temperature. The estimated kinetic solubility of Mesylate in water was visually determined to be ≥38 mg/mL.

### Sulfate

Only one crystalline form of the sulfate salt was observed during screening. Sulfate was produced from isopropyl alcohol and was successfully prepared at larger scale. Despite using both IC and EDX techniques, results were inconsistent and the stoichiometry was not confirmed. Based on IC, data suggests Sulfate may have a 2:3 2C-B:sulfuric acid stoichiometry as the reported sulfate content was 36.1%. This is relatively close to the theoretical content for a 2:3 stoichiometry, which is 35.4%. Conversely, the theoretical sulfate content for a 1:1 salt is only 26.8%. However, EDX data indicates that the ratio of bromine (used to represent 2C-B) to sulfur is 1:0.8. Further analyses using a sulfate ion probe or, ideally, solving the single crystal structure would be necessary to confirm the stoichiometry.

The TG thermogram for Sulfate shows a 0.2% weight loss from ambient to 160° C., indicating that the material is anhydrous (FIG. 131). Because there are no organic solvents by NMR, the weight loss is likely attributable to residual water and would be equivalent to less than 0.1 moles. The sharp endotherm in the DSC thermogram at 169° C. is believed to be due to melting.

Based on DVS data, Sulfate is slightly hygroscopic and gains 2.2% moisture through 95% RH (FIG. 143). Assuming a 1:1 stoichiometry, this would be equivalent to approximately 0.5 moles of water. Upon desorption, all the gained moisture is lost and form change does not occur.

Deliquescence was not observed after several days at 75% RH at ambient temperature. The estimated kinetic solubility of Sulfate in water was visually determined to be 14 mg/mL.

### Tartrate (Form 1)

Only one crystalline form of the tartrate salt was observed during screening. Tartrate (Form 1) was obtained from an ambient temperature slurry in ethanol and was successfully produced at larger scale. It has 1:1 stoichiometry. The TG thermogram only shows a 0.1% weight loss from ambient to 182° C., indicating that Tartrate (Form 1) is anhydrous (FIG. 132). Because there are no organic solvents by NMR, this is likely attributable to residual water and would be equivalent to just 0.03 moles. Melting is suspected to occur at 206° C.

Based on DVS data, Tartrate (Form 1) is non-hygroscopic and only gains 0.1% moisture through 95% RH (FIG. 144). Form change does not occur by XRPD.

Deliquescence was not observed after several days at 75% RH at ambient temperature. The estimated kinetic solubility of Tartrate (Form 1) in water was visually determined to be 11 mg/mL.

### Conclusions

A salt screen with 4-bromo-2,5-dimethoxyphenethylamine (2C-B) free base was conducted in order to identify a salt suitable for clinical development. Crystalline salts were generated with 2C-B and the following acids: benzenesulfonic acid, citric acid, ethanesulfonic acid, fumaric acid, gentisic acid, glutamic acid, glycolic acid, D,L-lactic acid, L-malic acid, maleic acid, methanesulfonic acid, mucic (galactaric) acid, phosphoric acid, succinic acid, sulfuric acid, L-tartaric acid, toluenesulfonic acid, and 1-hydroxy-2-naphthoic (xinafoic) acid.

Several salts were prepared at larger scale and fully characterized. These included Besylate (Form 1), Citrate (Form 2), Gentisate, Glycolate, Malate, Mesylate, Sulfate, and Tartrate (Form 1).

Of these, Tartrate (Form 1) is recommended for polymorph screening and further study. It is anhydrous and has 1:1 stoichiometry. It also has one of the highest melting points of the anhydrous salts at 206° C. Tartrate (Form 1) is non-hygroscopic and does not deliquesce at 75% RH. It has a high aqueous solubility (11 mg/mL). It is also reproducible and was readily prepared at gram quantities.

Gentisate would also be a good candidate. It is anhydrous, has 1:1 stoichiometry, and the highest melting point at 215° C. Gentisate is non-hygroscopic and does not deliquesce at 75% RH. It is also reproducible and was readily prepared at larger scale. However, the estimated aqueous solubility is only 2 mg/mL.

The besylate, citrate, and malate salts are not as highly recommended due to their potential for polymorphism. Despite having high aqueous solubility, the mesylate and glycolate salts were not selected due to likely hygroscopicity issues. The sulfate salt could be recommended; however, its stoichiometry has not been confirmed.

### Experimental

Preparation of 2C-B B.esylate (Form 1)

A solution of benzenesulfonic acid (201.5 mg, 1 eq) in 1 mL of ethanol was added to a solution of 2C-B free base (300.9 mg) in 6 mL of ethanol, resulting in precipitation. More solvent was added (approximately 5 mL) and the suspension was slurried at room temperature for 9 days. Solids were isolated via vacuum filtration, dried under vacuum at room temperature for 1 day, and analyzed by XRPD.

Preparation of 2C-B B.esylate (Form 2)

Benzenesulfonic acid (12.1 mg, 1 eq) was added to a solution of 2C-B free base (19.5 mg) in 1 mL of methyl ethyl ketone at room temperature, resulting in precipitation. The mixture was slurried at room temperature for 5 days, after which it was centrifuged and the mother liquor was decanted. The remaining solids were air-dried and analyzed by XRPD.

### Preparation of 2C-B Citrate (Form 1)

Citric acid (16.0 mg, 1 eq) was added to a suspension of 2C-B free base (21.8 mgin 1 mL of ethyl acetate at room temperature. The mixture was heated to approximately 50° C. and slurried at elevated temperature for 3 days, then cooled to room temperature. Stirring was continued for another 5 days, after which the sample was centrifuged and the mother liquor was decanted. The remaining solids were air-dried and analyzed by XRPD.

### Preparation of 2C-B Citrate (Form 1)—Alternate Procedure

Citric acid (13.1 mg, 1 eq) was added to a solution of 2C-B free base (18.0 mg) in 1 mL of methyl ethyl ketone, producing solids. The suspension was heated at 50° C. and slurried at elevated temperature for 5 days. It was then cooled to room temperature and the sample was centrifuged and the mother liquor was decanted. The remaining solids were air-dried and analyzed by XRPD.

### Preparation of 2C-B Citrate (Form 2)

Citric acid (249.4 mg, 1 eq) was added to a solution of 2C-B free base (336.4 mg) in 5 mL of ethyl acetate at 50° C. Solids slowly dissolved and then the mixture became cloudy. The suspension was slurried at 50° C. for 1 day, then cooled to room temperature. Stirring was continued for 4 days, after which solids were isolated via vacuum filtration, dried under vacuum at room temperature for 1 day, and analyzed by XRPD.

### Preparation of 2C-B Esylate (Form 1)

Ethanesulfonic acid (6.8 μL, 1 eq) was added to a solution of 2C-B free base (21.7 mg) in 1 mL of isopropanol at 50° C. Precipitation was not observed and the sample was cooled to room temperature. Stirring was continued for 10 days, during which time solids precipitated. The sample was then centrifuged and the mother liquor was decanted. The remaining solids were air-dried and analyzed by XRPD.

### Preparation of 2C-B Esylate (Form 2)

Ethanesulfonic acid (5.9 μL, 1 eq) was added to a suspension of 2C-B free base (18.7 mg) in 1 mL of methyl tert-butyl ether at 50° C., producing white solids. Added additional solvent (1 mL) and slurried at 50° C. for 7 days. The sample was then centrifuged and the mother liquor was decanted. The remaining solids were air-dried and analyzed by XRPD.

### Preparation of 2C-B Fumarate (Form 1)

Fumaric acid (8.0 mg, 1 eq) was added to a suspension of 2C-B free base (19.1 mg) in 1 mL of di-isopropyl ether at 50° C. The mixture was slurried at elevated temperature for 3 days, then cooled to room temperature. Stirring was continued for 5 days, after which the sample was centrifuged and the mother liquor was decanted. The remaining solids were air-dried and analyzed by XRPD.

### Preparation of 2C-B Fumarate (Form 1)—Alternate Procedure

Fumaric acid (9.1 mg, 1 eq) was added to a solution of 2C-B free base (20.2 mg) in 1 mL of acetonitrile. Solids persisted and the mixture was heated at 50° C., producing a thick suspension. Additional acetonitrile was added (0.5 mL) and the suspension was slurried at elevated temperature for 5 days. The sample was then centrifuged and the mother liquor was decanted. The remaining solids were air-dried and analyzed by XRPD.

### Preparation of 2C-B Gentisate

Gentisic acid (327.9 mg, 1 eq) was added to a solution of 2C-B free base (503.5 mg) in 13 mL of acetonitrile at 50° C., resulting in dissolution followed by precipitation. The suspension was slurried at 50° C. for 2 days, then cooled to room temperature. Stirring was continued for 3 days, after which solids were isolated via vacuum filtration, dried under vacuum at room temperature for 1 day, and analyzed by XRPD.

### Preparation of 2C-B Gentisate—Alternate Procedure

Gentisic acid (13.1 mg, 1 eq) was added to a solution of 2C-B free base (21.9 mg) in 1 mL of isopropyl acetate at 50° C., producing white solids. The suspension was cooled to room temperature and slurried for 8 days, after which the sample was centrifuged and the mother liquor was decanted. The remaining solids were air-dried and analyzed by XRPD.

### Preparation of 2C-B Glutamate

A solution of 2C-B free base (22.6 mg) in 0.5 mL of ethanol was added to a suspension of L-glutamic acid (13.1 mg, 1 eq) in 0.5 mL of water at 50° C., producing a clear solution. After a few hours, precipitation was not observed and the mixture was cooled to room temperature. Stirring was continued for 10 days, but solids were not produced. The solution was evaporated to dryness at room temperature and the resulting solids were collected and analyzed by XRPD.

### Preparation of 2C-B Glutamate—Alternate Procedure

L-glutamic acid (11.2 mg, 1 eq) and 2C-B free base (19.0 mg) were combined in a Retsch milling container with a stainless-steel ball. 10 μL each of methanol and water were added and the sample was milled at 25 Hz for 30 minutes. Solids were air-dried and analyzed by XRPD.

### Preparation of 2C-B Glycolate

Glycolic acid (97.7 mg, 1 eq) was added to a mostly clear solution of 2C-B free base (331.3 mg) in 5-7 mL of methyl tert-butyl ether at 50° C., resulting in precipitation of peach-colored solids. Additional solvent was added (3-5 mL) and the suspension was slurried at 50° C. for 1 day, then cooled to room temperature. Stirring was continued for 4 days, after which the solids were isolated via vacuum filtration, dried under vacuum at room temperature for 1 day, and analyzed by XRPD.

### Preparation of 2C-B Glycolate—Alternate Procedure

Glycolic acid (6.0 mg, 1 eq) was added to a solution of 2C-B free base (20.5 mg) in 1 mL of isopropyl acetate at 50° C., producing white solids. The suspension was cooled to room temperature and slurried for 8 days, after which the sample was centrifuged and the mother liquor was decanted. The remaining solids were air-dried and analyzed by XRPD.

### Preparation of 2C-B Lactate (Form 1)

D,L-lactic acid (85%, 5.8 μL, 1 eq) was added to a solution of 2C-B free base (17.8 mgin 1 mL of isopropyl acetate at 50° C., resulting in a clear solution. The mixture was stirred at room temperature for 8 days, during which time solids precipitated. The sample was then centrifuged and the mother liquor was decanted. The remaining solids were air-dried and analyzed by XRPD.

### Preparation of 2C-B Lactate (Form 2)

D,L-lactic acid (85%, 5.8 μL, 1 eq) was added to a solution of 2C-B free base (18.0 mg) in 1 mL of isopropanol, producing a clear solution. The mixture was stirred at elevated temperature for 7 days. It was then cooled to room temperature and the sample was centrifuged and the mother liquor was decanted. The remaining solids were air-dried and analyzed by XRPD.

### Preparation of 2C-B Malate

A hot solution of 2C-B free base (406.3 mg) in 5 mL of methyl ethyl ketone at 50° C. was added to a suspension of L-malic acid (230.5 mg, 1 eq) in 2 mL of methyl ethyl ketone at 50° C., resulting in precipitation. The suspension was slurried at 50° C. for 7 days, then cooled to room temperature. Solids were isolated via vacuum filtration, dried under vacuum for 1 day, and analyzed by XRPD.

### Preparation of 2C-B Malate—Alternate Procedure

L-malic acid (12.0 mg, 1 eq) was added to a suspension of 2C-B free base (22.5 mg) in 1 mL of ethyl acetate. Solids persisted and the mixture was heated at 50° C. and slurried for 3 days at elevated temperature. The sample was then cooled to room temperature and stirring was continued for 5 days, after which it was centrifuged and the mother liquor was decanted. The remaining solids were air-dried and analyzed by XRPD.

### Preparation of 2C-B Maleate (Form 1)

Maleic acid (10.0 mg, 1 eq) was added to a solution of 2C-B free base (22.4 mg) in 1 mL of di-isopropyl ether at 50° C., producing solids. The suspension was slurried at elevated temperature for 3 days. It was then cooled to room temperature and the stirring was continued for 5 days, after which the sample was centrifuged and the mother liquor was decanted. The remaining solids were air-dried and analyzed by XRPD.

### Preparation of 2C-B Maleate (Form 2)

Maleic acid (8.0 mg, 1 eq) was added to a solution of 2C-B free base (18.5 mg) in 1 mL of methyl ethyl ketone, producing solids. The suspension was slurried at room temperature for 7 days, after which it was centrifuged and the mother liquor was decanted. The remaining solids were air-dried and analyzed by XRPD.

### Preparation of 2C-B Mesylate

Methanesulfonic acid (117.5 μL, 1 eq) was added to a solution of 2C-B free base (428.2 mg) in 5 mL of acetonitrile at 50° C., resulting in a clear solution. The mixture was stirred at 50° C. for 7 days, during which time solids precipitated. The suspension was cooled to room temperature and the solids were isolated via vacuum filtration, dried under vacuum at room temperature for 1 day, and analyzed by XRPD.

### Preparation of 2C-B Mesylate—Alternate Procedure

Methanesulfonic acid (4.5 μL, 1 eq) was added to a suspension of 2C-B free base in 1.5 mL of methyl tert-butyl ether at 50° C., producing white solids. The mixture was slurried at elevated temperature for 4 days. The sample was then cooled to room temperature and the stirring continued for 6 days, after which it was centrifuged and the mother liquor was decanted. The remaining solids were air-dried and analyzed by XRPD.

### Preparation of 2C-B Mucate

Mucic (galactaric) acid (14.2 mg, 1 eq) was added to a solution of 2C-B free base (17.2 mg) in 1 mL of acetonitrile. Solids persisted and the mixture was heated at 50° C. and slurried for 3 days at elevated temperature. The sample was then cooled to room temperature and stirring was continued for 5 days, after which it was centrifuged and the mother liquor was decanted. The remaining solids were air-dried and analyzed by XRPD.

### Preparation of 2C-B Mucate—Alternate Procedure

Mucic (galactaric) acid (17.9 mg, 1 eq) was added to a solution of 2C-B free base (22.3 mg) in 1 mL of ethanol. Solids persisted and the mixture was heated at 50° C. and slurried at elevated temperature for 10 days. The sample was then centrifuged, the mother liquor was decanted, and the remaining solids were air-dried and analyzed by XRPD.

### Preparation of 2C-B Phosphate (Form 1)

Phosphoric acid (85%, 5.9 μL, 1 eq) was added to a suspension of 2C-B free base (22.6 mg) in 1 mL of methyl tert-butyl ether at 50° C., producing white solids. Additional solvent was added (0.5 mL) and the mixture was slurried at elevated temperature for 4 days. The sample was then cooled to room temperature and the stirring continued for 6 days, after which it was centrifuged and the mother liquor was decanted. The remaining solids were air-dried and analyzed by XRPD.

### Preparation of 2C-B Phosphate (Form 2)

Phosphoric acid (85%, 5.6 μL, 1 eq) was added to a suspension of 2C-B free base (21.4 mg) in 1 mL of isopropanol at 50° C., producing white solids. Additional solvent was added (1 mL) and the mixture was slurried at elevated temperature for 10 days. The sample was then centrifuged, the mother liquor was decanted, and the remaining solids were air-dried and analyzed by XRPD.

### Preparation of 2C-B Sulfate

Sulfuric acid (72 μL, 1 eq) was added to a solution of 2C-B free base in 13 mL of isopropanol, resulting in precipitation. Additional solvent was added (2 mL) and the suspension was slurried at room temperature for 9 days. Solids were isolated via vacuum filtration, dried under vacuum at room temperature for 1 day, and analyzed by XRPD.

### Preparation of 2C-B Sulfate—Alternate Procedure

Sulfuric acid (4.5 μL, 1 eq) was added to a suspension of 2C-B free base in 1.5 mL of methyl tert-butyl ether at 50° C., producing white solids. The mixture was slurried at elevated temperature for 4 days. The sample was then cooled to room temperature and the stirring continued for 6 days, after which it was centrifuged and the mother liquor was decanted. The remaining solids were air-dried and analyzed by XRPD.

### Preparation of 2C-B Succinate (Form 1)

Succinic acid (9.0 mg, 1 eq) was added to a solution of 2C-B free base (20.6 mg) in 1 mL of isopropanol at 50° C., producing white solids. The suspension was then cooled to room temperature and additional solvent was added (1 mL). The mixture was slurried at room temperature for 7 days, after which it was centrifuged and the mother liquor was decanted. The remaining solids were air-dried and analyzed by XRPD.

### Preparation of 2C-B Tartrate (Form 1)

L-tartaric acid (318.9 mg, 1 eq) was added to a solution of 2C-B free base (502.9 mg) in 12 mL of ethanol, resulting in precipitation. More solvent was added (3 mL) and the suspension was slurried at room temperature for 5 days. Solids were isolated via vacuum filtration, dried under vacuum at room temperature for 1 day, and analyzed by XRPD.

### Preparation of 2C-B Tartrate (Form 1)—Alternate Procedure

L-tartaric acid (11.8 mg) was added to a solution of 2C-B free base (20.3 mg) in 0.5 mL of acetone, producing a gel. Additional solvent was added (0.5 mL) and the mixture was heated at 50° C., with stirring. The gel solidified and the mixture was slurried at elevated temperature for 4 days. The sample was then cooled to room temperature and the stirring continued for 6 days, after which it was centrifuged and the mother liquor was decanted. The remaining solids were air-dried and analyzed by XRPD.

### Preparation of 2C-B Tosylate (Form 1)

Toluenesulfonic acid hydrate (15.4 mg, 1 eq) was added to a suspension of 2C-B free base (20.9 mg) in 0.5 mL of ethyl acetate. Additional solvent was added (1 mL) and the suspension was slurried at room temperature for 10 days. The sample was then centrifuged, the mother liquor was decanted, and the remaining solids were air-dried and analyzed by XRPD.

### Preparation of 2C-B Xinafoate

1-hydroxy-2-napthoic acid (14.1 mg, 1 eq) was added to a suspension of 2C-B free base (19.0 mg) in 1 mL of di-isopropyl ether at 50° C. The suspension was slurried at elevated temperature for 8 days, after which the sample was centrifuged and the mother liquor was decanted. The remaining solids were air-dried and analyzed by XRPD.

### Preparation of 2C-B Xinafoate—Alternate Procedure

1-hydroxy-2-napthoic acid (14.1 mg, 1 eq) was added to a solution of 2C-B free base (19.4 mg) in 1 mL of isopropyl acetate, producing solids. The suspension was slurried at room temperature for 5 days, after which it was centrifuged and the mother liquor was decanted. The remaining solids were air-dried and analyzed by XRPD.

### Instrumental Techniques

### X-Ray Powder Diffraction (XRPD)

A Rigaku SmartLab X-Ray Diffractometer was configured in Bragg-Brentano reflection geometry equipped with a beam stop and knife edge to reduce incident beam and air scatter. Data collection parameters are shown in the following table.

PXRD Data Collection Parameters

| Parameter | Value | Parameter | Value |
|---|---|---|---|
| Geometry | Bragg-Brentano | Receiving Slit 1 (mm) | 18 |
| Tube Anode | Cu | Receiving Slit 2 (mm) | open |
| Tube Type | Long Fine Focus | Start Angle 2θ (°) | 2 |
| Tube Voltage (kV) | 40 | End Angle 2θ (°) | 40 |
| Tube Current (mA) | 44 | Step Size (°) | 0.02 |
| Detector | D/teX Ultra 250 | Scan Speed (°/min) | 6 |
| | HyPix-3000 | | |
| Monochromator | Ni foil Cu Kβ Filter | Spinning (rpm) | 11 |
| Incident Slit (°) | ⅓ | Sample Holder | Low-background Si |

Differential Scanning calorimetry (DSC)

The DSC analyses were carried out using a TA Instruments Q2500 Discovery Series instrument. The instrument temperature calibrations were performed using indium. The DSC cell was kept under a nitrogen or helium purge of ˜50 mL per minute during each analysis. Each sample was placed in a standard, crimped aluminum pan and headed from approximately 25° C. to 300° C. at a rate of 10° C. per minute.

### Thermogravimetric (TG) Analysis

The TG analyses were carried out using a TA Instruments Q5500 Discovery Series instrument. The instrument balance was calibrated using class M weights and the temperature calibration was performed using alumel. The nitrogen or helium purge was ˜10 mL per minute at the balance and ˜25 mL per minute at the furnace. Each sample was placed into a pre-tared platinum pan and heated from approximately 25° C. to 300° C. at a rate of 10° C. per minute.

### Dynamic Vapor Sorption (DVS) Analysis

The DVS analyses were carried out using a TA Instruments Q5000 Dynamic Vapor Sorption analyzer. Approximately 7-11 mg of sample was loaded into a metal-coated quartz pan for analysis. After equilibration at 5% relative humidity (RH), the sample was analyzed at 25° C. in 10% RH steps from 5 to 95% RH (adsorption cycle) and from 95 to 5% RH (desorption cycle). The movement from one step to the next occurred either after satisfying the equilibrium criterion of 0.01% weight change in 5 minutes or, if the equilibrium criterion was not met, after 90 minutes. The percent weight change values were calculated using Microsoft Excel®.

### Nuclear Magnetic Resonance (NMR) Spectroscopy

The ¹H NMR spectra were acquired on a Bruker Avance II 400 spectrometer. Samples were prepared by dissolving material in DMSO-d₆ and/or d-MeOD. The solutions were placed into individual 5-mm NMR tubes for subsequent spectral acquisition. The temperature controlled (295K) ¹H NMR spectra acquired on the Avance II 400 utilized a 5-mm cryoprobe operating at an observing frequency of 400.18 MHz.

### Optical Microscopy (OM)

Optical microscopy experiments were carried out on a Leica DM 2500 P compound microscope. Images were captured using a PAXcam3 camera and were collected at 20× or 40× magnification.

### Scanning Electron Microscopy (SEM)

Samples were sputter coated with gold to improve image quality and decrease sample damage. SEM images were collected using a Phenom XL Desktop SEM equipped with a CeB₆ electron source. Phenom User Interface version 1.4 was used to acquire and save the images. Detailed image parameters are recorded in the footer of each image.

### Energy Dispersive X-Ray Spectroscopy (EDX)

EDX analysis was conducted using a Phenom XL Benchtop scanning electron microscope (SEM) equipped with a thermoelectrically cooled silicon drift detector (SDD) for EDX analysis. An ultra-thin silicon nitride (Si₃N₄) X-ray window allowed detection of elements with atomic numbers ranging from 4 to 95. Solids were mounted to an aluminum SEM stub with double-sided carbon tape. The instrument was configured in backscatter electron detection (BSD) mode with a 6-7 mm working distance. The beam voltage was adjusted to optimize the signal intensity while avoiding charging at the sample surface. Phenom User Interface version 1.4 was used to acquire and save the images and spectral data. EDX data show both the atomic percent and the weight percent of the detected elements.

Polymorph Screening

### Summary

A polymorph screen of 4-bromo-2,5-dimethoxyphenethylamine (2C-B) tartrate was conducted in order to evaluate its polymorphic landscape and identify a solid form for clinical development. Previously, a salt screen performed identified one form of the tartrate salt, Tartrate (Form 1). Tartrate (Form 1) is a crystalline, anhydrous salt with 1:1 stoichiometry. Melting occurs at 206° C.

In addition to Tartrate (Form 1), one unique material was observed during the polymorph screen. It was designated as 2C-B Tartrate (Form 2). Tartrate (Form 2) is a crystalline, anhydrous salt with 1:1 stoichiometry. Melting likely occurs at 200° C. Tartrate (Form 2) was originally produced from the scale-up experiment targeting Tartrate (Form 1), an ambient temperature slurry in ethanol, and was therefore used as starting material for the majority of polymorph screen experiments. Tartrate (Form 2) was also observed a few times throughout the screen, typically from ester-containing solvents or alcohols.

Non-crystalline 2C-B tartrate was also prepared and characterized. Though a glass transition was not observed, it was found that the material recrystallizes to Tartrate (Form 1) upon heating.

Tartrate (Form 1) is likely the more thermodynamically stable form, under the conditions tested in this study, as the majority of experiments generated solids consistent with Tartrate (Form 1) by XRPD. In particular, all solids isolated from long-term slurry experiments, with the exception of those from ethyl acetate, were consistent with Tartrate (Form 1). Note that these slurries were conducted across a wide range of temperatures (5° C. to 50° C.). These results indicate that Tartrate (Form 2) readily converts to Tartrate (Form 1) in most solvents and under most conditions.

Based on polymorph screen results, Tartrate (Form 1) appears to be the more stable form under conditions tested and would be recommended for clinical development.

### Background

A salt screen of 4-bromo-2,5-dimethoxyphenethylamine (2C-B) free base identified several crystalline salts. A salt screen of a selected salt and the tartrate salt was chosen for further screening. 2C-B tartrate is a crystalline, anhydrous salt with 1:1 stoichiometry. Melting occurs at 206° C. It is non-hygroscopic and has an aqueous solubility of 11 mg/mL. 2C-B and its salts are DEA Schedule I controlled substances and potent materials.

The goal of this study was to conduct a polymorph screen with 2C-B tartrate and identify and characterize any polymorphs.

### Results and Discussion

Polymorph Screening

Additional quantities of the tartrate salt were prepared to use as starting material for the polymorph screen. To this end, one molar equivalent of maleic acid was added to a suspension of 2C-B free base (2C-B free base was prepared from the HCl salt) in ethanol. The mixture was slurried at ambient temperature for several days. Details and results are provided in Table 78.

TABLE 78
Preparation of 2C-B Tartrate

| | XRPD | |
|---|---|---|
| Conditions^{a} | Results | FIG. No. |
| To a sol'n of 2C-B FB (1.6 g) in 30 mL EtOH | Tartrate | 65 |
| was added L-tartaric acid (1 eq). The mixture | (Form 2) | |
| was sonicated using a sonication bath, | | |
| resulting in dissolution of the acid followed | | |
| by formation of a solid plug. ~20 mL EtOH | | |
| was added and the suspension was slurried at | | |
| RT for 4 days. The solid was isolated via | | |
| vacuum filtration and dried in a vacuum | | |
| desiccator at RT overnight (2.0 g, 80% yield). | | |

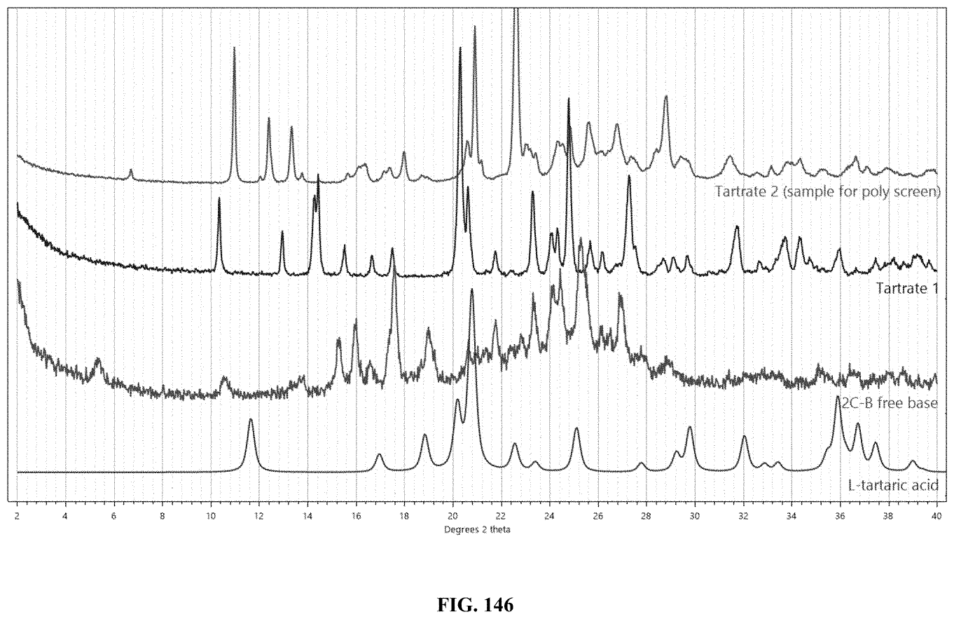

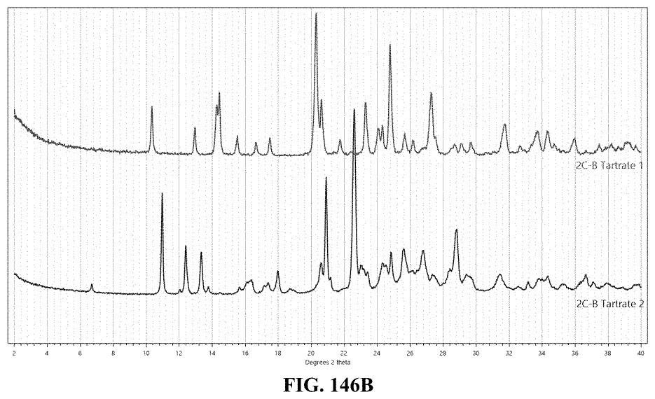

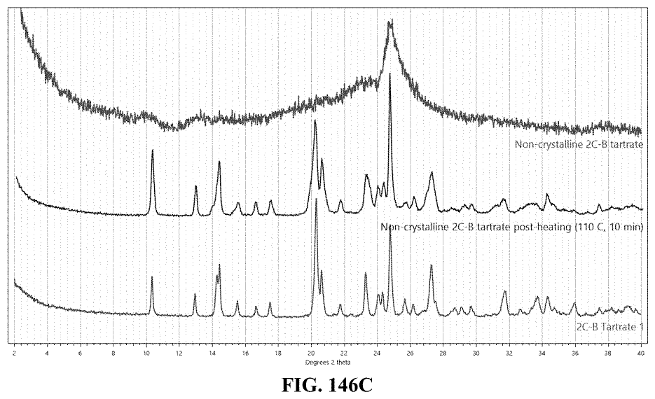

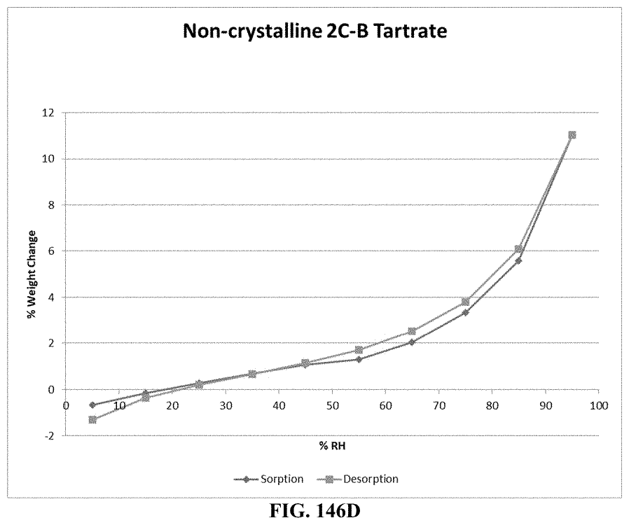

^{a}FB = free base; sol'n = solution; EtOH = ethanol, g = grams, eq = equivalent; RT = room/ambient temperature
XRPD patterns of 2C-B tartrate polymorphs are provided in FIG. 146.

Solubilities of 2C-B tartrate in a few solvents were estimated. The experiments were carried out by adding the test solvent in aliquots to weighed portions of solid. Whether dissolution had occurred was judged by visual inspection after addition of each solvent aliquot. The results are shown in Table 79. Solubility numbers were calculated by dividing the weight of the sample by the total amount of solvent used to dissolve the sample. The actual solubilities may be greater than the numbers calculated because of the use of solvent aliquots that were too large or because of slow dissolution rates. Solubilities are reported as “<” if dissolution did not occur during the experiment. All solubility measurements were carried out at room temperature.

TABLE 79
Estimated Solubilities of 2C-B Tartrate

| Solvent^{a} | Solubility (mg/mL) |
|---|---|
| Acetone | <1 |
| ACN | <1 |
| CHCl₃ | <1 |
| DMF | 4 |
| EtOH | <1 |
| EtOH/H₂O 50/50 | 10 |
| IPA/H₂O 80/20 | 7 |
| IPA/H₂O 95/5 | <2 |
| MeOH | 6 |
| THF | <1 |

^{a}ACN = acetonitrile, CHCl₃ = chloroform, DMF = dimethyl formamide, EtOH = ethanol, IPA = isopropanol, H₂O = water, MeOH = methanol, THF = tetrahydrofuran

2C-B Tartrate was mixed with various solvents under various conditions in attempts to generate polymorphs. Solvent choice was based on a Class 2 or 3 rating as well as the solubilities of 2C-B tartrate. Because the primary goal was to determine the stable form of 2C-B tartrate, experiments mainly incorporated thermodynamic-based techniques such as slow cooling, anti-solvent addition, and long-term slurry. Several experiments were done in the presence of water, including high water activity slurries, in an effort to look for hydrates. Experiments and results are summarized in Table 81.

TABLE 81
Samples Generated and Analyzed

| Method | Solvent^{a} | Conditions^{b} | XRPD Results^{c} |
|---|---|---|---|
| Evaporation | EtOH/H₂O 50/50 | RT | IS |
| | IPA/H₂O 80/20 | RT | Tartrate (Form 1); |
| | | | LC |
| | MeOH | RT | Tartrate (Form 1) |
| | | | (PO) |
| Precipitation | EtOH | RT | Tartrate (Form 1) |
| | EtOH/heptane | 50° C. | Tartrate (Form 1) |
| | | | (PO) + small 23° |
| | | | pk |
| | EtOH/iPrOAc | 50° C. | Tartrate (Form 1) + |
| | | | trace Tartrate |
| | | | (Form 2) |
| | H₂O/acetone | RT−>5° C. | Tartrate (Form 1) |
| | | | (PO) |
| | H₂O/ACN | RT−>5° C.; NS. | — |
| | H₂O/i-BuOH | RT−>5° C.; NS. | — |
| | H₂O/IPA | RT−>5° C. | Tartrate (Form 1) |
| | | | (PO) |
| | H₂O/1-PrOH | RT−>5° C.; NS. | — |
| | MeOH/CHCl₃ | RT−>5° C. | Tartrate (Form 1) |
| | MeOH/iPrOAc | RT | Tartrate (Form 1) |
| | MeOH/2-Me THF | RT−>5° C. | Tartrate (Form 1) |
| | MeOH/MIBK | RT−>5° C. | Tartrate (Form 1) |
| | MeOH/MTBE | RT | Tartrate (Form 1) |
| Cooling | acetone | 50° C.−>5° C. | Tartrate (Form 2) |
| | ACN | 50° C.−>5° C. | IS |
| | ACN/H₂O | 50° C.−>RT | Tartrate (Form 1) + |
| | | | pks |
| | ACN/MeOH | 50° C.−>RT | Tartrate (Form 1) |
| | CHCl₃ | 50° C.−>5° C.; NS. | — |
| | CHCl₃/MeOH | 50° C.−>5° C. | Tartrate (Form 1) |
| | dioxane/H₂O | 50° C.−>RT | Tartrate (Form 1) + |
| | | | pks |
| | dioxane/MeOH | 50° C.−>RT | Tartrate (Form 1) |
| | EtOAc/MeOH | 50° C.−>RT | Tartrate (Form 1) + |
| | | | small 19° pk |
| | EtOH/acetone | 50° C.−>5° C.; NS. | — |
| | EtOH/ACN | 50° C.−>5° C. | Tartrate (Form 1) |
| | EtOH/CHCl₃ | 50° C.−>RT | Tartrate (Form 1) |

^{a}EtOH = ethanol; MeOH = methanol; IPA = isopropanol; H₂O = water; iPrOAc =
isopropyl acetate; ACN = acetonitrile; i-BuOH = isobutanol; 1-PrOH = 1-propanol;
CHCl₃ = chloroform; 2-Me THF = 2-methyl tetrahydrofuran; MIBK = methyl isobutyl
ketone; MTBE = methyl tert-butyl ether; EtOAc = ethyl acetate; DEE = diethyl ether
^{b}RT = room/ambient temperature
^{c}NC = non-crystalline; PO = preferred orientation; pk(s) = peak(s); IS = insufficient
solids

| Method | Solvent^{a} | Conditions^{b} | XRPD Results^{c} |
|---|---|---|---|
| Cooling | EtOH/DEE | 50° C.−>RT | Tartrate (Form 1) |
| | EtOH/EtOAc | 50° C.−>RT | Tartrate (Form 1) |
| | EtOH/heptane | 50° C.−>RT | Tartrate (Form 1) (PO) |
| | EtOH/hexanes | 50° C.−>RT | Tartrate (Form 1) |
| | EtOH/IPE | 50° C.−>RT | Tartrate (Form 1) |
| | EtOH/iPrOAc | 50° C.−>RT | Tartrate (Form 1) |
| | EtOH/2-Me THF | 50° C.−>RT | Tartrate (Form 1) |
| | EtOH/MEK | 50° C.−>5° C.; NS. | — |
| | EtOH/MIBK | 50° C.−>5° C. | Tartrate (Form 1) |
| | EtOH/MTBE | 50° C.−>RT | Tartrate (Form 1) |
| | EtOH/toluene | 50° C.−>RT | Tartrate (Form 1) |
| | | | (PO) |
| | IPA/H₂O 95/5 | 50° C.−>RT | Tartrate (Form 1) |
| | IPA/H₂O | 50° C.−>RT | Tartrate (Form 1) + |
| | | | pks(13°, 19°) |
| | IPE/MeOH | 50° C.−>RT | Tartrate (Form 1) |
| | iPrOAc/MeOH | 50° C.−>RT | Tartrate (Form 1) |
| | MEK/MeOH | 50° C.−>RT | Tartrate (Form 1) |
| | MIBK/MeOH | 50° C.−>5° C.; NS. | — |
| | MTBE/MeOH | 50° C.−>RT; single | Tartrate (Form 1) |
| | | crystals | |
| | THF/H2O | 50° C.−>5° C. | Tartrate (Form 1) |
| Slurry | ACN | 50° C., 9 days | Tartrate (Form 1) |
| | DMF/acetone 50/50 | RT | Tartrate (Form 1) |
| | EtOAc | 50° C., 9 days | Tartrate (Form 1) |
| | EtOAc/DMSO 71/29 | RT | Tartrate (Form 1) + |
| | | | small pks |
| | EtOH | 50° C., 9 days | Tartrate (Form 1) |
| | IPA | 50° C., 9 days | Tartrate (Form 1) |
| | MEK | 50° C., 9 days | Tartrate (Form 1) |
| | MeOH | RT | Tartrate (Form 1) |
| | acetone/H₂O 50/50 | 5° C. | Tartrate (Form 1) |
| | a_{w} = 0.91¹ | | |
| | EtOH/H₂O 50/50 | RT | Tartrate (Form 1) |
| | a_{w} = 0.89 | | |

^{a}EtOH = ethanol; MeOH = methanol; IPA = isopropanol; H₂O = water; iPrOAc =
isopropyl acetate; ACN = acetonitrile; CHCl₃ = chloroform; 2-Me THF = 2-methyl
tetrahydrofuran; MIBK = methyl isobutyl ketone; MTBE = methyl tert-butyl ether;
EtOAc = ethyl acetate; DEE = diethyl ether; IPE = di-isopropyl ether; MEK =
methyl ethyl ketone; THF = tetrahydrofuran; DMF = dimethyl formamide; DMSO =
dimethyl sulfoxide; a_{w} = water activity
^{b}RT = room/ambient temperature
^{c}NC = non-crystalline; PO = preferred orientation; pk(s) = peak(s); IS = insufficient
solids

| Method | Solvent^{a} | Conditions^{b} | XRPD Results^{c} |
|---|---|---|---|
| Slurry | EtOH/H₂O 80/20 | RT | Tartrate (Form 1) |
| | a_{w} = 0.70 | | |
| | EtOH/H₂O 90/10 | RT | Tartrate (Form 1) |
| | a_{w} = 0.49 | | |
| | IPA/H₂O 70/30 | RT | Tartrate (Form 1) |
| | a_{w} = 0.94 | | |
| | MeOH/H₂O 95/5 | RT | Tartrate (Form 1) |
| | a_{w} = 0.16 | | |
| | THF/H₂O 50/50 | 5° C. | Tartrate (Form 1) |
| | a_{w} = 1.00 | | |
| Lyophilization | dioxane/H₂O | −50° C.−>RT | NC + br pk (23°) |

^{a}EtOH = ethanol; MeOH = methanol; IPA = isopropanol; H₂O = water; THF =
tetrahydrofuran; a_{w} = water activity
^{b}RT = room/ambient temperature
^{c}NC = non-crystalline; br = broad; pk(s) = peak(s)

Non-crystalline 2C-B tartrate was used as starting material for some experiments. This was done because using a non-crystalline sample can sometimes provide access to polymorphs that would not be obtained from a crystalline sample.

Non-crystalline 2C-B tartrate was prepared from lyophilization. Samples were subsequently treated with various crystallization techniques as described in Table 83.

TABLE 83
Polymorph Screen Experiments with Non-crystalline 2C-B Tartrate

| Method | Solvent^{a} | Conditions^{b} | XRPD Results^{c} |
|---|---|---|---|
| Liquid Vapor | IPA/CHCl₃ | RT−>5° C.; NS. | — |
| Diffusion | IPA/DEE | RT | Tartrate (Form 1) |
| | IPA/EtOAc | RT | Tartrate (Form 1) |
| | IPA/hexanes | RT | Tartrate (Form 1) |
| | IPA/IPE | RT−>5° C.; NS. | — |
| | IPA/iPrOAc | RT | IS |
| | IPA/MIBK | RT−>5° C.; NS. | — |
| | IPA/MTBE | RT−>5° C.; NS. | — |
| | MeOH/DEE | RT | Tartrate (Form 1) |
| | MeOH/heptane | RT | Tartrate (Form 1) |
| Humidity | — | 93% RH, RT | Tartrate (Form 1) |
| stress | | | |
| Heat stress | — | 60° C., 9 d | NC + br pk (23°) |
| | — | 110° C., 10 min. | Tartrate (Form 1) + |
| | | | 19.8° shoulder |
| Vapor stress | acetone | RT | Tartrate (Form 1) |
| | EtOH | RT | Tartrate (Form 1) |
| | H₂O | RT | Tartrate (Form 1) |

^{a}IPA = isopropanol; MeOH = methanol; CHCl₃ = chloroform; DEE = diethyl ether; EtOAc = ethyl acetate; IPE = di-isopropyl ether; iPrOAc = isopropyl acetate; MIBK = methyl iso-butyl ketone; MTBE = methyl tert-butyl ether; EtOH = ethanol; H₂O = water
^{b}RT = room/ambient temperature; NS = no solids; RH = relative humidity; d = day(s); min. = minutes
^{c}IS = insufficient solids; NC = non-crystalline; br = broad; pk(s) = peak(s)

2C-B free base was also used as an alternative starting material for crystallization of the tartrate salt. This was done in order to do reaction crystallization experiments with L-tartaric acid as these types of experiments can provide access to polymorphs of the tartrate salt that may not otherwise be obtained. Preparation of the free base is described in Table 84. Reaction crystallization experiments and results are summarized in Table 85A.

TABLE 84
Preparation of 2C-B Free Base

| | XRPD |
|---|---|
| Conditions^{a} | Results^{b} |
| Added 4.3 mL of 1N NaOH to a solution of 604 mg | FB 1 + |
| of 2C-B HCl (sample TCL16246) and 2C-B tartrate | NC |
| (sample 1226-23-01) in 44 mL of water (cloudy, | |
| then oiling observed). Stirring, RT, 1 d. Extracted | |
| w/EtOAc (3x). Combined organic layers and dried | |
| w/MgSO₄. Evaporated w/stream of air. | |

^{a}NaOH = sodium hydroxide;
EtOAc = ethyl acetate;
MgSO₄ = magnesium sulfate;
RT = room/ambient temperature;
d = day(s);
w/ = with
^{b}FB = 2C-B free base;
NC = non-crystalline

TABLE 85A
Reaction Crystallization Experiments with 2C-B Free Base and L-Tartaric Acid

| Solvent^{a} | Conditions^{b} | XRPD Results^{c} |
|---|---|---|
| ACN/H₂O | Added acid to sol'n of FB in ACN (0.5 mL) at | Tartrate (Form 1) |
| | 50° C.; solids persisted. Added 1 drop of H₂O; | |
| | clear then solids pp'd. Added ACN; SL, 50° C., 4 | |
| | days. | |
| EtOAc/MeOH | Added acid to sol'n of FB in EtOAc at 50° C.; | Tartrate (Form 1) |
| | solids persisted. Added MeOH; solids slowly | |
| | pp'd. SL, 50° C., 4 days. | |
| | Added acid to suspension of FB in EtOAc at RT. | Tartrate (Form 2) |
| | Added MeOH; solids slowly pp'd. SL, RT, 4 | |
| | days. | |
| IPA/H₂O | Added acid to sol'n of FB in IPA (1 mL) at RT; | Tartrate (Form 1); LC |
| | solids persisted. Added 1 drop of H₂O; clear | |
| | then solids pp'd. Added more IPA; SL, RT, 4 | |
| | days. | |
| IPE/MeOH | Added acid to suspension of FB in IPE | Tartrate (Form 2) + |
| | (1 mL) at 50° C.; some solids persisted. Added | Tartrate (Form 1) |
| | MeOH (~100 μL); solids pp'd. Added IPE; SL, | |
| | 50° C., 4 days. | |
| iPrOAc/MeOH | Added acid to suspension of FB in iPrOAc (1 | Tartrate (Form 1) |
| | mL) at 50° C.; solids persisted. Added MeOH | |
| | (~100 μL); solids slowly pp'd. SL, 50° C., 4 | |
| | days. | |
| MEK | Added acid to solution of FB in MEK | Tartrate (Form 1) |
| | (1 mL) at RT; clear gel. Heated at 50° C.; solids | |
| | slowly pp'd. Added MEK; SL, 50° C., 4 days. | |
| MeOH | Added acid to sol'n of FB in MeOH at RT; clear | Tartrate (Form 1) |
| | then solids pp'd. SL, RT, 4 days. | |
| THF/H₂O | Added acid to sol'n of FB in THF (1 mL) at RT; | Tartrate (Form 1) |
| | solids persisted. Added 1 drop of H₂O; clear | |
| | then solids pp'd. Added more THF; SL, RT, 4 | |
| | days. | |

^{a}ACN = acetonitrile; EtOAc = ethyl acetate; MeOH = methanol; IPA = isopropanol; IPE = di-isopropyl ether; H₂O = water; iPrOAc = isopropyl acetate; MeOH = methanol; MEK = methyl ethyl ketone; THF = tetrahydrofuran
^{b}FB = 2C-B free base; SL = slurry; RT = room/ambient temperature; pp'd = precipitated
^{c}LC = low crystallinity

In addition to Tartrate (Form 1), one polymorph was identified during screening. It was designated as Tartrate (Form 2). The XRPD pattern is shown below in FIG. 146B.

Tartrate (Form 2) and non-crystalline 2C-B tartrate were further characterized by proton nuclear magnetic resonance spectroscopy (¹H NMR), thermogravimetric analysis (TGA), and differential scanning calorimetry (DSC). The non-crystalline material was also characterized by modulated DSC and dynamic vapor sorption (DVS) with XRPD of the post-DVS solids. Data is summarized in Table 85B.

TABLE 85B
Characterization of New Materials

| | Analytical | | |
|---|---|---|---|
| Sample | Technique^{a} | Results^{b} | FIG. No. |
| 2C-B Tartrate (Form | ¹H NMR | Consistent with structure | 106 |
| 2) | | Peak shifts consistent w/ salt formation | |
| | | 1:1 stoichiometry | |
| | | 0.1 moles EtOH | |
| | TGA | 0.2%, start to 156° C. | 133 |
| | DSC | Endo: 200° C., 202° C. | |
| Non-crystalline | ¹H NMR | Consistent with structure | |
| 2C-B tartrate | | 0.1 moles dioxane | |
| | TGA | 4.6%, start to 122.4° C. | 134 |
| | DSC | Endo: 94° C., 204° C. | |
| | | Exo: 99° C. | |
| | mDSC | Parameters: | 157 |
| | | Cool to −50° C.; isotherm for 2 min. | |
| | | Modulate +/−0.320° C. every 30 secs | |
| | | Ramp 4.00° C./min to 250° C. | |
| | | Results: | |
| | | T_{g}: Not observed | |
| | | Exo: 106° C. | |
| | | Endo: 197° C. | |
| | | Parameters: | |
| | | Heat 20° C./min to 90° C. | |
| | | Cool 20° C./min to −50° C.; isotherm for 2 min. | |
| | | Modulate +/−0.320° C. every 30 secs | |
| | | Ramp 20° C./min to 250° C. | |
| | | Results: | |
| | | T_{g}: Not observed | |
| | | Exo: 116° C. | |
| | DVS | Sorption: | |
| | | 5-55% RH: 2.0% gain | |
| | | 55-95% RH: 9.7% gain | |
| | | Total gain: 11.7% | |
| | | Desorption: | |
| | | 95-5% RH: 12.3% loss | |
| | post-DVS | NC + br pk (25°) | |
| | XRPD | | |

^{a1}H NMR = proton nuclear magnetic resonance spectroscopy; TGA = thermogravimetric analysis; DSC = differential scanning calorimetry; mDSC = modulated differential scanning calorimetry; DVS = dynamic vapor sorption; XRPD = X-ray powder diffraction
^{b}EtOH = ethanol; Endo = endotherm; Exo = exotherm; T_{g} = glass transition temperature; isotherm = isothermal; min = minute; sec(s) = second(s); RH = relative humidity; NC = non-crystalline; br = broad; pk(s) = peak(s)

Tartrate (Form 2) is a crystalline salt with 1:1 stoichiometry. It appears to be anhydrous as there is only residual ethanol (0.1 moles) in the ¹H NMR spectrum and a 0.2% weight loss from ambient to 156° C. in the TG thermogram (FIG. 133). The two endotherms present at 200° C. and 202° C. in the DSC thermogram could be due to melting and may indicate Tartrate (Form 2) undergoes form conversion upon heating. Note that the endotherms coincide with the beginning of a large weight loss in the TG thermogram, which suggests that melting is concurrent with decomposition. However, it is also possible that, given the large weight loss, these endotherms could be due to sublimation instead of melting.

Tartrate (Form 2) was produced from the scale-up experiment targeting Tartrate (Form 1), an ambient temperature slurry in ethanol, and was used as starting material for the majority of screening experiments.

During the screen, Tartrate (Form 2) was observed from an elevated temperature slurry in ethyl acetate, a cooling experiment involving acetone, and in trace amounts as a mixture with Tartrate (Form 1) from an ethanol/isopropyl acetate precipitation experiment at elevated temperature. It was also produced from reaction crystallization experiments using ethyl acetate/methanol and di-isopropyl ether/methanol.

Tartrate (Form 1) is likely the more thermodynamically stable form, under the conditions tested in this study, as the majority of experiments generated solids consistent with Tartrate (Form 1) by XRPD. In particular, all solids isolated from long-term slurry experiments, with the exception of those from ethyl acetate, were consistent with Tartrate (Form 1). Note that these slurries were conducted across a wide range of temperatures (5° C. to 50° C.). These results indicate that Tartrate (Form 2) readily converts to Tartrate (Form 1) in most solvents and under most conditions.

Non-crystalline 2C-B tartrate was prepared via lyophilization. The XRPD pattern of the non-crystalline material displays diffuse scattering and broad haloes and does not contain peaks indicative of crystallinity. The material is structurally intact by NMR and has 1:1 stoichiometry. Though the NMR spectrum only contains residual dioxane (0.1 moles), the TG thermogram shows a 4.6% weight loss from ambient to 122° C., which is accompanied by a broad endotherm in the DSC thermogram at 94° C. (FIG. 134). Assuming 2.1% of the weight loss is due to the 0.1 moles of dioxane, the remaining 2.5% is likely attributable to approximately 0.6 moles of water.

Note the exotherm present at 99° C. in the DSC thermogram, as well as a second endotherm at 204° C. (FIG. 134). The exotherm is typical of a recrystallization event and the endotherm is likely due to melting. Based on the temperature at which it occurs, it could be attributable to the melt of Tartrate (Form 1). These events suggest that the non-crystalline material recrystallizes to Tartrate (Form 1) upon heating. Indeed, after non-crystalline tartrate was heated at 110° C. for 10 minutes, crystallization to Tartrate (Form 1) was observed by XRPD (FIG. 146C).

A glass transition temperature was not observed for the non-crystalline material, even when the analysis was repeated with a drying cycle (FIG. 135B).

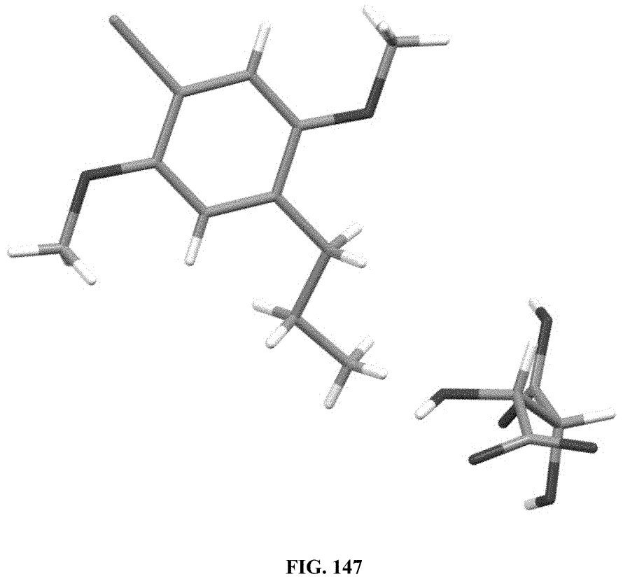

Based on DVS data, non-crystalline 2C-B tartrate is hygroscopic and gains 11.7% moisture through 95% RH (FIG. 147D). The majority of this (9.7%) occurs above 55% RH. Upon desorption, the sample loses all the gained moisture and remains non-crystalline, by XRPD.

The kinetic aqueous solubility was visually estimated to be 7 mg/mL. Note that this is lower than the crystalline tartrate salt, which had a solubility of 11 mg/mL, and is unexpected given that non-crystalline materials tend to have higher solubility, at least initially.

### Conclusions

A polymorph screen of 2C-B tartrate was conducted in order to evaluate the polymorphic landscape and identify a solid form for clinical development. One form of the tartrate salt, Tartrate (Form 1), was previously known. Tartrate (Form 1) is a crystalline, anhydrous salt with 1:1 stoichiometry. Melting occurs at 206° C.

In addition to Tartrate (Form 1), one polymorph was identified during the screen. It was designated as Tartrate (Form 2). Like Tartrate (Form 1), Tartrate (Form 2) is crystalline with 1:1 stoichiometry. Tartrate (Form 2) is also anhydrous and likely melts at 200° C.

Tartrate (Form 2) was originally produced from the scale-up experiment targeting Tartrate (Form 1), which was an ambient temperature slurry in ethanol. It was also observed a few times throughout the screen, typically from ester-containing solvents or alcohols.

Non-crystalline 2C-B tartrate was also prepared and characterized. Though a glass transition was not observed, it was found that the material recrystallizes to Tartrate (Form 1) upon heating.

Tartrate (Form 1) is likely the more thermodynamically stable form, under the conditions tested in this study, as the majority of experiments generated solids consistent with Tartrate (Form 1) by XRPD. In particular, all solids isolated from long-term slurry experiments, with the exception of those from ethyl acetate, were consistent with Tartrate (Form 1). Note that these slurries were conducted across a wide range of temperatures (5° C. to 50° C.). These results indicate that Tartrate (Form 2) readily converts to Tartrate (Form 1) in most solvents and under most conditions.

Overall, Tartrate (Form 1) appears to be the more stable form under conditions tested and is recommended for clinical development.

### Experimental

### Typical Slurry Experiment

2C-B tartrate was suspended in ethanol and heated at approximately 50° C. The suspension was slurried at elevated temperature for 9 days. The sample was then centrifuged, while still warm, and the mother liquor was decanted. The isolated solids were air-dried and analyzed by XRPD.

### Typical Precipitation Experiment

2C-B tartrate was dissolved in approximately 3 mL of methanol, resulting in a clear solution. The solution was filtered (0.45 μm nylon) into a new vial and approximately 10 mL of chloroform was slowly added. The solution remained clear and the sample was transferred to a refrigerator (approximately 5° C.) for 9 days, during which time precipitation occurred. The mother liquor was decanted and the isolated solids were air-dried and analyzed by XRPD.

### Typical Cooling Experiment

2C-B tartrate was suspended in approximately 5-7 mL of acetonitrile and heated at approximately 50° C., with stirring. Approximately 3-5 mL of methanol were added at elevated temperature, producing a slightly turbid solution. The solution was hot-filtered (0.45 μm nylon) into a pre-heated vial and the hot plate was turned off to slowly cool the sample to ambient temperature. Precipitation occurred and the supernatant was decanted. The isolated solids were air-dried and analyzed by XRPD.

### Typical Evaporation Experiment

2C-B tartrate (5.4 mg) was dissolved in 900 μL of methanol, resulting in a clear solution. The sample was left uncapped, but covered with aluminum foil with one pinhole, at ambient temperature. The resulting solids were collected and analyzed by XRPD.

### Typical Lyophilization Experiment

2C-B tartrate was dissolved in a mixture of dioxane and water. The solution was filtered (0.45 μm nylon) and transferred into a 50 mL round bottom flask. The flask was rotated in a dry-ice acetone bath to freeze the solution to the walls of the flask. The flask was then placed on a Labconco FreeZone 1 lyophilizer, pre-equilibrated at ˜50° C., resulting in white, fluffy solids. The solids were collected and analyzed by XRPD.

### Typical Liquid Vapor Diffusion Experiment

2C-B tartrate was lyophilized. The lyophilized material was then dissolved in isopropanol, resulting in a clear solution. The solution was transferred into a 1-dram vial and the vial was placed, uncapped, into a 20-mL vial containing diethyl ether. The 20-mL vial was capped and the sample was kept at ambient temperature for approximately 2 weeks, during which time solids precipitated. The 1-dram vial was removed and the solvent was decanted. The isolated solids were air-dried and analyzed by XRPD.

### Typical Vapor Stress Experiment

2C-B tartrate was lyophilized. The lyophilized material was then transferred into a 1-dram vial. The vial was placed, uncapped, in ajar containing acetone. The jar was capped and the sample was kept at ambient temperature for approximately 8 days. The remaining solids were collected and analyzed by XRPD.

### Typical Heat Stress Experiment

Solids of non-crystalline 2C-B tartrate were heated in an oven at approximately 110° C. for 10 minutes. They were then removed, cooled to ambient temperature, and analyzed by XRPD.

### Humidity Stress Experiment

2C-B tartrate was lyophilized. The lyophilized material was then transferred into a 1-dram vial. The vial was placed, uncapped, in ajar containing a saturated salt solution, designed to maintain a relative humidity of approximately 93%. The jar was capped and the sample was kept at ambient temperature for approximately 8 days. The remaining solids were collected and analyzed by XRPD.

### Typical Reaction Crystallization Experiment

L-tartaric acid (10.0 mg, 1 eq) was added to a suspension of 2C-B free base (16.9 mg, sample 1285-07-01) in 1 mL of isopropyl acetate at 50° C. Approximately 100 μL of methanol were added dropwise and solids slowly began to precipitate. The mixture was slurried at elevated temperature for 4 days. The sample was then centrifuged and the mother liquor was decanted. The isolated solids were air-dried and analyzed by XRPD.

### Instrumental Techniques

### X-ray Powder Diffraction (XRPD)

A Rigaku SmartLab X-Ray Diffractometer was configured in Bragg-Brentano reflection geometry equipped with a beam stop and knife edge to reduce incident beam and air scatter. Data collection parameters are shown in the table below.

PXRD Data Collection Parameters

| Parameter | Value | Parameter | Value |
|---|---|---|---|
| Geometry | Bragg-Brentano | Receiving Slit 1 (mm) | 18 |
| Tube Anode | Cu | Receiving Slit 2 (mm) | open |
| Tube Type | Long Fine Focus | Start Angle 2θ (°) | 2 |
| Tube Voltage (kV) | 40 | End Angle 2θ (°) | 40 |
| Tube Current (mA) | 44 | Step Size (°) | 0.02 |
| Detector(s) | D/teX Ultra 250 | Scan Speed (°/min) | 6 |
| | HyPix-3000 | | |
| Monochromator | Ni foil Cu Kβ Filter | Spinning (rpm) | 11 |
| Incident Slit (°) | ⅓ | Sample Holder | Low-background Si |

Differential Scanning calorimetry (DSC)

The DSC analyses were carried out using a TA Instruments Q2500 Discovery Series instrument. The instrument temperature calibrations were performed using indium. The DSC cell was kept under a nitrogen purge of ˜50 mL per minute during the analyses. Each sample was placed in a standard, crimped aluminum pan and heated from approximately 25° C. to 300° C. at a rate of 10° C. per minute.

### Thermogravimetric (TG) Analysis

The TG analysis was carried out using a TA Instruments Q5500 Discovery Series instrument. The instrument balance was calibrated using class M weights and the temperature calibration was performed using alumel. The nitrogen purge was ˜10 mL per minute at the balance and ˜25 mL per minute at the furnace. The sample was placed into a pre-tared platinum pan and heated from approximately 25° C. to 300° C. at a rate of 10° C. per minute.

Modulated Differential Scanning calorimetry (mDSC)

The first modulated DSC analysis was carried out using a TA Instruments Q2000 Discovery Series instrument. The DSC cell was kept under a nitrogen purge of ˜50 mL per minute during the analysis. The sample was placed in a standard, crimped aluminum pan and cooled to −50° C. and held at this temperature for 2 minutes. It was then modulated +32° C. every seconds while being heated to 250° C. at a rate of 4° C. per minute.

The second modulated DSC analysis was carried out using a Q2500 Discovery Series instrument. The instrument temperature calibrations were performed using indium. The DSC cell was kept under a nitrogen purge of ˜50 mL per minute during the analysis. The sample was placed in a standard, crimped aluminum pan and heated to 90° C. at a rate of 20° C. per minute. It was then cooled to −50° C. and held at this temperature for 2 minutes. It was then modulated +32° C. every 30 seconds while being heated to 250° C. at a rate of 20° C. per minute.

### Dynamic Vapor Sorption (DVS) Analysis

The DVS analysis was carried out using a TA Instruments Q5000 Dynamic Vapor Sorption analyzer. Approximately 2 mg of sample was loaded into a metal-coated quartz pan for analysis. After equilibration at 5% relative humidity (RH), the sample was analyzed at 25° C. in 10% RH steps from 5 to 95% RH (adsorption cycle) and from 95 to 5% RH (desorption cycle). The movement from one step to the next occurred either after satisfying the equilibrium criterion of 0.01% weight change in 5 minutes or, if the equilibrium criterion was not met, after 90 minutes. The percent weight change values were calculated using Microsoft Excel®.

### Nuclear Magnetic Resonance (NMR) Spectroscopy

The ¹H NMR spectra were acquired on a Bruker Avance II 400 spectrometer. Samples were prepared by dissolving material in DMSO-d₆. The solutions were placed into individual 5-mm NMR tubes for subsequent spectral acquisition. The temperature controlled (295K)¹H NMR spectra acquired on the Avance II 400 utilized a 5-mm cryoprobe operating at an observing frequency of 400.18 MHz.

### Example 6-2C-B Salt Selection

All twenty-one acids used in the salt screening produced solids with a unique XRPD pattern. Several of these appeared to be reproducible as a second experiment gave solids with the same XRPD pattern. The materials that were made twice with the same XRPD pattern and had the highest crystallinity were considered lead candidates (Table 86). Additional characterization of the top salts resulted in the tartrate salt exhibiting acceptable scale-up (no other forms observed), acceptable water uptake (minimal water uptake over RH range), uncomplicated DSC data (one endothermic event observed) and acceptable solubility for development when compared to the other salts.

TABLE 86
Matrix Table for 2C-B Salt Selection

| Salt/Property | Tartrate (Form 1) | | Gentisate | | Sulfate | | Glycolate | | Malate | |
|---|---|---|---|---|---|---|---|---|---|---|
| XRPD | crystalline | | crystalline | | crystalline | | crystalline | | crystalline | |
| TGA volatile | 0.1 (at 182 C.) | | 1.2 (178 C.) | | 0.2 (156 C.) | | 0.9 (125 C.) | | 0.1 (146 C.) | |
| content % | | | | | | | | | | |
| Designation | anhydrate | | anhydrate | | anhydrate | | anhydrate | | anhydrate | |
| Stoichiometry | 1:1 | | 1:1 | | 2:3 | | 1:1 | | 1:1 | |
| NMR-salt | consistent with | | consistent with | | consistent with | | consistent with | | consistent with | |
| formation | structure | | structure | | structure | | structure and | | structure | |
| | | | | | | | salt formation | | | |
| NMR solvent | no solvent | | no solvent | | no solvents | | 0.1 mol MTBE | | no solvent | |
| DSC transition | endotherm | | endotherms | | endotherm | | Endotherm | | endotherm | |
| (maximum peak | 206 | | 124, 215 | | 57, 169 | | 149, 161, | | 151 | |
| temperature C.) | | | | | | | exo 151 | | | |
| Water uptake (25- | non- | | non- | | moderately | | slighly | | slighly | |
| 65% RH) | hygroscopic | | hygroscopic | | hygroscopic | | hygroscopic | | hygroscopic | |
| Water uptake (5- | 0.1 | | 0.20% | | 2.2 (total); 0.8 | | 3.6 (total); 0.9 | | 1.6 (total), 0.6 | |
| 95% RH) % | | | | | (5-85), 1.4 (85-95) | | (5-85), 2.7 (85-95) | | (5-85); 1 (85-95) | |
| Deliquescence 75% | no | | no | | no | | NA | | no | |
| RH/1 d | deliquescence | | deliquescence | | deliquescence | | | | deliquescence | |
| Kinetic water | 11 | mg/mL | 2 | mg/mL | 14 | mg/mL | >45 | mg/mL | 6 | mg/mL |
| solubility (mg/mL) | | | | | | | | | | |
| Scale-up (300-500 mg) | no issues | | no issues | | no issues | | no issues | | no issues | |
| Particle size | ~25 | um | ~25 | um | <15 | um | <15 | um | <15 | um |
| (optical microscopy) | | | | | | | | | | |

| | | | | | |
|---|---|---|---|---|---|
| Morphology | irregular, rods | rods and | irregulare, | aggregates | irregular |
| optical microscopy | and plates | plates | flake-like | | aggregates |
| Mophology SEM | aggregates, | rods and | aggregates, | NA | NA |
| | rods and | plates, minor | rods and | | |
| | plates | crystals on | plates | | |
| | | surface | | | |

### Example 7—Single Crystal Structure of 4-Bromo-2,5-Dimethoxyphenethylamine (2C-B) Tartrate (Form 1)

### Summary

The crystal structure of 4-bromo-2,5-dimethoxyphenethylamine (2C-B) tartrate was solved. The tartrate salt structure was determined to be anhydrous with 1:1 2C-B:tartaric acid stoichiometry and formula C₁₀H₁₅BrNO₂.C₄H₅O₆. The calculated powder pattern from the single crystal data matched the X-ray powder diffraction (XRPD) pattern of the bulk sample.

### Background

A salt screen of 4-bromo-2,5-dimethoxyphenethylamine (2C-B) was performed. A tartrate salt was identified and a polymorph screen was subsequently conducted on the material. Single crystals suitable for structure determination were observed in one of the samples and the structure was solved.

### Results and Discussion

A sample of 2C-B tartrate was examined by optical microscopy and appeared to contain single crystals of sufficient size and quality. The sample was submitted to the crystallographer at Purdue University, who selected and mounted one crystal, collected diffraction data, and solved the structure. The bulk sample was also measured by XRPD.

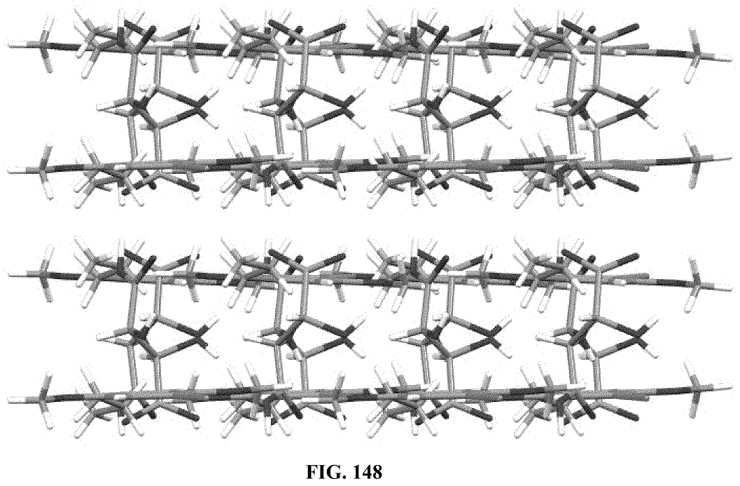

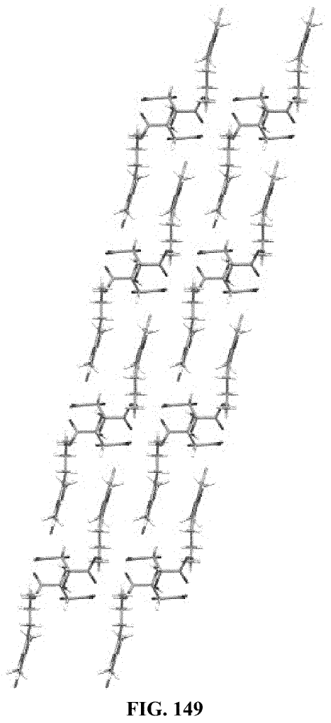

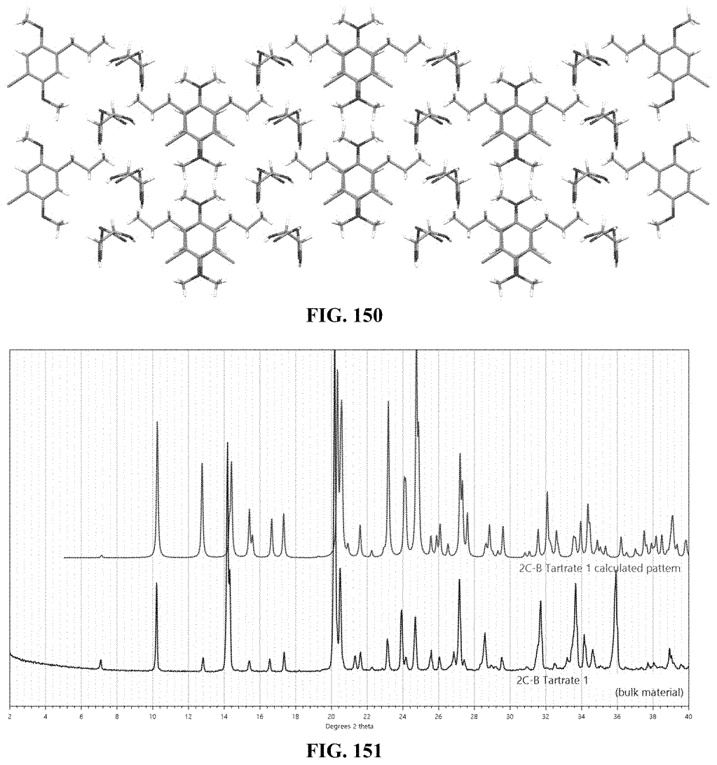

Unit cell parameters are shown in Table 87A. The asymmetric unit is shown in FIG. 147. Packing diagrams along the a, b, and c axes are shown in FIG. 148, FIG. 149, and FIG. 150.

TABLE 87A
Unit cell parameters of 2C-B tartrate

| Crystal system, space group | monoclinic, C2 (5) |
|---|---|
| Data collection temperature (K) | 150(2) |
| a (Å) | 25.4494(15) |
| b (Å) | 9.2035(6) |
| c (Å) | 7.3803(5) |
| volume (Å³) | 1683.95(19) |
| Z | 4 |

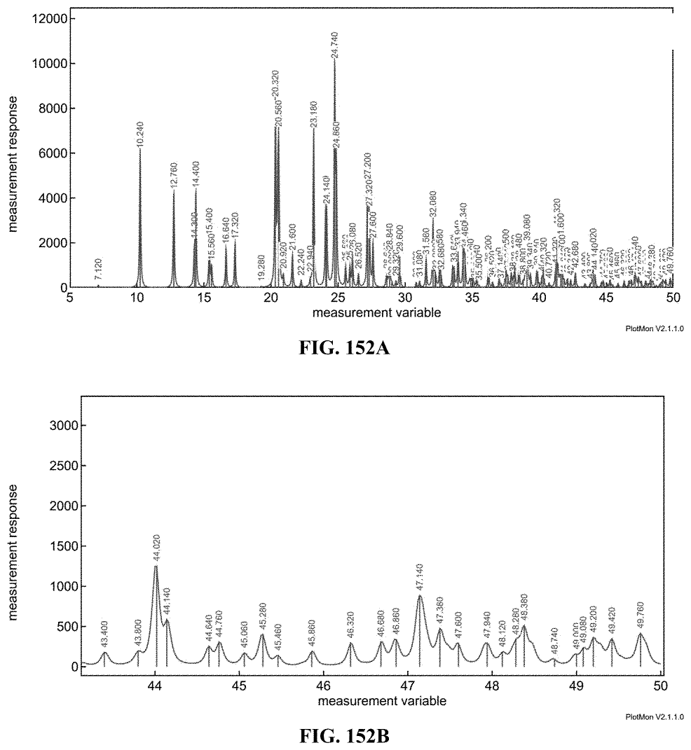

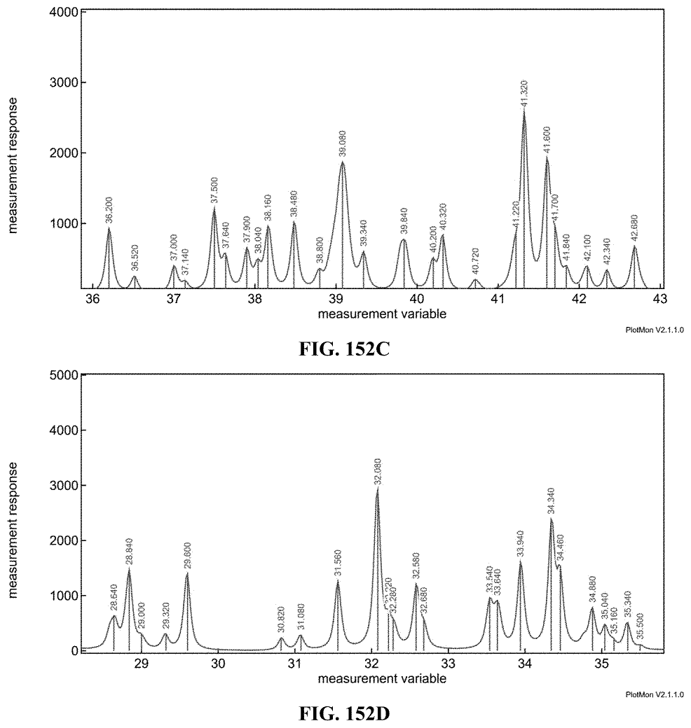

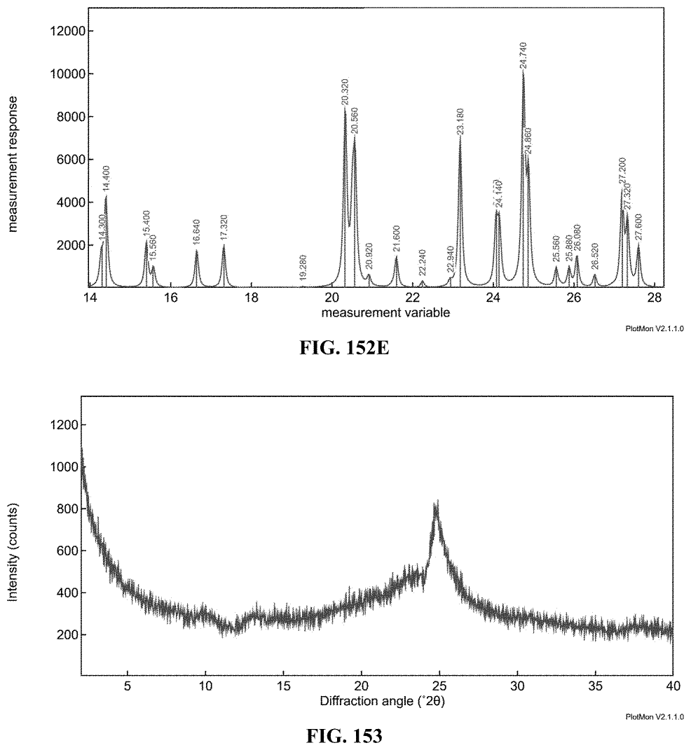

An XRPD pattern calculated from the single-crystal data is overlaid with a pattern obtained for in FIG. 151, and overall matches the XRPD pattern of the bulk material. The observed peak shifting is due to the temperature difference at which the single crystal and X-ray powder diffraction data were collected. The XRPD pattern of 2C-B tartrate (Form 1) calculated from single-crystal data is provided in FIGS. 152A-E. The XRPD Signal angle data of 2C-B Tartrate (Form 1) calculated from single-crystal data are provided in Table 87B.

TABLE 87B
XRPD Signal angle data of 2C-B Tartrate (Form
1) calculated from single-crystal data

| Position (°2θ) | Relative |
|---|---|
| 7.1 | 1 |
| 10.2 | 60 |
| 12.8 | 42 |
| 14.3 | 20 |
| 14.4 | 42 |
| 15.4 | 22 |
| 15.6 | 10 |
| 16.6 | 17 |
| 17.3 | 20 |
| 19.3 | 1 |
| 20.3 | 83 |
| 20.6 | 70 |
| 20.9 | 6 |
| 21.6 | 14 |
| 22.2 | 3 |
| 22.9 | 5 |
| 23.2 | 69 |
| 24.1 | 36 |
| 24.1 | 35 |
| 24.7 | 100 |
| 24.9 | 60 |
| 25.6 | 10 |
| 25.9 | 10 |
| 26.1 | 15 |
| 26.5 | 6 |
| 27.2 | 46 |
| 27.2 | 46 |
| 27.3 | 34 |
| 27.6 | 20 |
| 28.6 | 6 |
| 28.8 | 15 |
| 29.0 | 3 |
| 29.3 | 3 |
| 29.6 | 14 |
| 30.8 | 2 |
| 31.1 | 3 |
| 31.6 | 13 |
| 32.1 | 29 |
| 32.2 | 8 |
| 32.3 | 6 |
| 32.6 | 12 |
| 32.7 | 6 |
| 33.5 | 10 |
| 33.6 | 9 |
| 33.9 | 16 |
| 34.3 | 24 |
| 34.5 | 15 |
| 34.9 | 8 |
| 35.0 | 5 |
| 35.2 | 2 |
| 35.3 | 5 |
| 35.5 | 1 |
| 36.2 | 9 |
| 36.5 | 3 |
| 37.0 | 4 |
| 37.1 | 2 |
| 37.5 | 12 |
| 37.5 | 12 |
| 37.6 | 6 |
| 37.9 | 7 |
| 38.0 | 5 |
| 38.2 | 10 |
| 38.5 | 10 |
| 38.8 | 4 |
| 39.1 | 19 |
| 39.3 | 6 |
| 39.8 | 8 |
| 40.2 | 5 |
| 40.3 | 8 |
| 40.7 | 2 |
| 41.2 | 9 |
| 41.3 | 26 |
| 41.6 | 19 |
| 41.7 | 10 |
| 41.8 | 4 |
| 42.1 | 4 |
| 42.3 | 3 |
| 42.7 | 7 |
| 43.4 | 2 |
| 43.8 | 2 |
| 44.0 | 13 |
| 44.1 | 6 |
| 44.6 | 3 |
| 44.8 | 3 |
| 45.1 | 2 |
| 45.3 | 4 |
| 45.5 | 1 |
| 45.9 | 2 |
| 46.3 | 3 |
| 46.7 | 3 |
| 46.9 | 3 |
| 47.1 | 9 |
| 47.4 | 5 |
| 47.6 | 3 |
| 47.9 | 3 |
| 48.1 | 2 |
| 48.3 | 3 |
| 48.4 | 5 |
| 48.7 | 1 |
| 49.0 | 2 |
| 49.1 | 2 |
| 49.2 | 4 |
| 49.4 | 3 |
| 49.8 | 4 |

### Experimental

### Single Crystal Growth

To 2C-B tartrate (˜20 mg) was added ˜6-9 mL of methyl tert-butyl ether. The cloudy suspension was heated at ˜50° C., using a hot plate. Approximately 5-7 mL of methanol was slowly added at elevated temperature until a slight turbidity remained. The mixture was then hot-filtered (0.45 μm nylon) into a pre-heated vial and the hot plate turned off to slowly cool to ambient temperature. The sample was kept at ambient temperature for four days, during which time precipitation occurred. Crystals suitable for structure determination were observed and isolated from solution. For the remaining sample, the supernatant was decanted and solids were briefly air-dried. Solids were rendered with a spatula prior to XRPD analysis.

### Single Crystal X-ray Structure Determination

A colorless plate shaped crystal with formula C₁₀H₁₅BrNO₂.C₄H₅O₆ having approximate dimensions of 0.033×0.100×0.110 mm was mounted on a Mitegen micromesh mount in a random orientation. Data were collected from a shock-cooled single crystal at 150(2) K on a Bruker AXS D8 Quest four circle diffractometer with an I-mu-S microsource X-ray tube using a laterally graded multilayer (Goebel) mirror as monochromator and a PhotonIII_C14 charge-integrating and photon counting pixel array detector. The diffractometer used CuKα radiation (λ=1.54178 Å). All data were integrated with SAINT V8.40B and a multi-scan absorption correction using SADABS 2016/2 was applied. ([1] Bruker, SAINT, V8.40B, Bruker AXS Inc., Madison, Wis., USA.; [2] L. Krause, R. Herbst-Irmer, G. M. Sheldrick, D. Stalke, J. Appl. Cryst. 2015, 48, 3-10, doi:10.1107/S1600576714022985.) The structure was solved by dual methods with SHELXT and refined by full-matrix least-squares methods against F² using SHELXL-2018/3. ([1] G. M. Sheldrick, Acta Cryst. 2015, A71, 3-8, doi:10.1107/52053273314026370; [2] G. M. Sheldrick, Acta Cryst. 2015, C71, 3-8, doi:10.1107/52053229614024218) All non-hydrogen atoms were refined with anisotropic displacement parameters. Carbon bound hydrogen atoms and ammonium H atoms were refined isotropically on calculated positions using a riding model. Methyl CH₃ and ammonium NH₃⁺ were allowed to rotate but not to tip to best fit the experimental electron density. Positions of hydroxyl H atoms were freely refined. Uᵢₛₒ values were constrained to 1.5 times the U_{eq} of their pivot atoms for terminal sp^{a} carbon atoms, hydroxyl groups and ammonium NH₃⁺ groups and 1.2 times for all other hydrogen atoms.

Crystal data and structure refinement parameters are given in Table 88.

TABLE 88
Experimental Details
Crystal Data

| Empirical formula | C₁₄H₂₀BrNO₈ |
|---|---|
| Moiety formula | C₁₀H₁₅BrNO₂•C₄H₅O₆ |
| Formula weight | 410.22 |
| Temperature [K] | 150(2) |
| Crystal system | monoclinic |
| Space group (number) | C2 (5) |
| a [Å] | 25.4494(15) |
| b [Å] | 9.2035(6) |
| c [Å] | 7.3803(5) |
| α [°] | 90 |
| β [°] | 103.057(2) |
| γ [°] | 90 |
| Volume [Å³] | 1683.95(19) |
| Z | 4 |
| ρ_{calc} [gcm⁻³] | 1.618 |
| μ [mm⁻¹] | 3.724 |
| F(000) | 840 |
| Crystal size [mm³] | 0.033 × 0.100 × 0.110 |
| Crystal colour | colourless |
| Crystal shape | plate |
| Radiation | CuK_{α} (λ = 1.54178 Å) |
| 2θ range [°] | 7.13 to 160.34 (0.78 Å) |
| Index ranges | −31 ≤ h ≤ 32 |
| | −11 ≤ k ≤ 11 |
| | −9 ≤ l ≤ 7 |
| Reflections collected | 14728 |
| Independent reflections | 3539 |
| | Rᵢₙₜ = 0.0334 |
| | R_{sigma} = 0.0315 |
| Completeness to θ = 67.679° | 99.3% |
| Data/Restraints/Parameters | 3539/1/230 |
| Goodness-of-fit on F² | 1.075 |
| Final R indexes [I ≥ 2σ(I)] | R₁ = 0.0233 |
| | wR₂ = 0.0540 |
| Final R indexes [all data] | R₁ = 0.0238 |
| | wR₂ = 0.0544 |
| Largest peak/hole [eÅ⁻³] | 0.69/−0.75 |
| Flack X parameter | 0.068(8) |

### X-Ray Powder Diffraction (XRPD)

A Rigaku SmartLab X-Ray Diffractometer was configured in Bragg-Brentano reflection geometry equipped with a beam stop and knife edge to reduce incident beam and air scatter. Data collection parameters are shown in Table 89.

TABLE 89
PXRD Data Collection Parameters

| Parameter | Value | Parameter | Value |
|---|---|---|---|
| Geometry | Bragg-Brentano | Receiving Slit 1 (mm) | 18 |
| Tube Anode | Cu | Receiving Slit 2 (mm) | 20 |
| Tube Type | Long Fine Focus | Start Angle 2θ (°) | 2 |
| Tube Voltage (kV) | 40 | End Angle 2θ (°) | 40 |
| Tube Current (mA) | 45 | Step Size (°) | 0.02 |
| Detector | HyPix-3000 | Scan Speed (°/min) | 6 |
| Monochromator | Nifoil Cu Kβ Filter | Spinning (rpm) | 11 |
| Incident Slit (°) | ⅓ | Sample Holder | Low-background Si |

### Example 8—Preparation and Characterization of Non-Crystalline 2C-B Tartrate

Non-crystalline 2C-B tartrate was prepared via lyophilization.

Procedure: To ˜100 mg of 2C-B tartrate was added dioxane and water (˜10 mL total), giving a clear solution. The solution was filtered (0.45 μm nylon) into a 50 mL round bottom flask. The flask was rotated in a dry-ice acetone bath to freeze the solution to the walls of the flask. The flask was then placed on a Labconco FreeZone 1 lyophilizer, pre-equilibrated at ˜50° C., overnight. The resulting white, fluffy solids were collected.

The aqueous kinetic solubility was visually estimated to be 7 mg/mL. Key/differentiating characteristics:

(a) A glass transition was not identified.
(b) Upon heating at ˜110° C. for 10 minutes, the non-crystalline material crystallized to 2C-B Tartrate (Form 1). This was based on XRPD data from a heating experiment done during screening. The endotherm at 116° C. in the mDSC is likely the recrystallization event.
(c) The non-crystalline material is hygroscopic and gained 11.7% moisture through 95% RH. The majority of this (9.7%) occurred above 55% RH. Upon desorption, the sample lost all the gained moisture and remained non-crystalline, by XRPD. This shows the non-crystalline material has some physical stability.
(d) The material handles well at ambient temperature (not sticky or staticky). This is not often the case for non-crystalline solids.

Non-crystalline 2C-B tartrate was characterized by X-ray powder diffraction (XRPD), proton nuclear magnetic resonance spectroscopy (1H NMR), thermogravimetric analysis (TGA), differential scanning calorimetry (DSC), modulated DSC, and dynamic vapor sorption (DVS) with XRPD of the post-DVS sample.

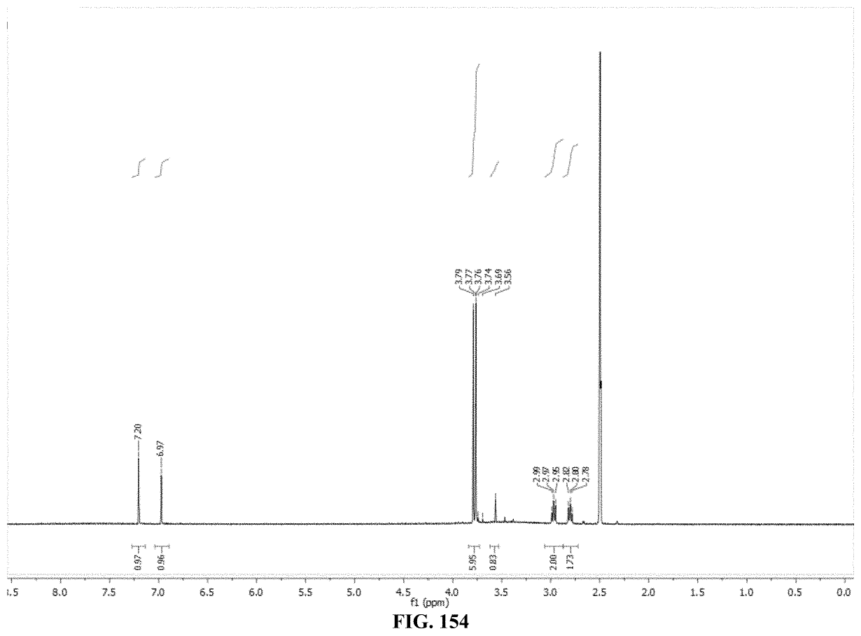

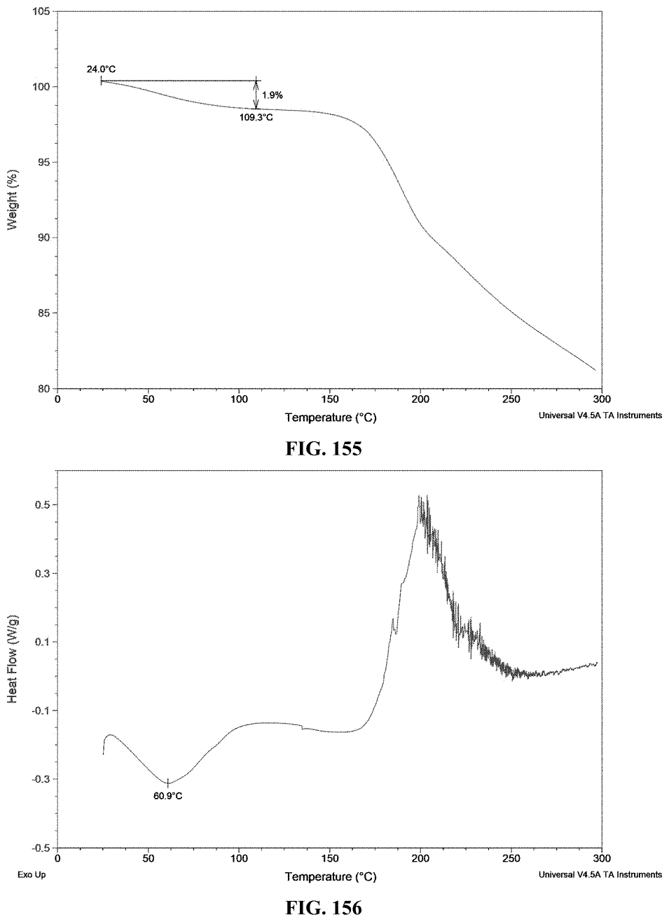

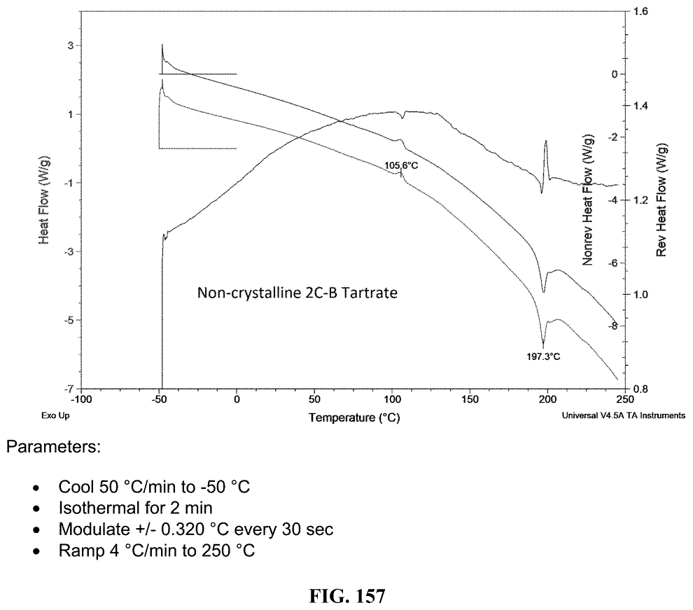

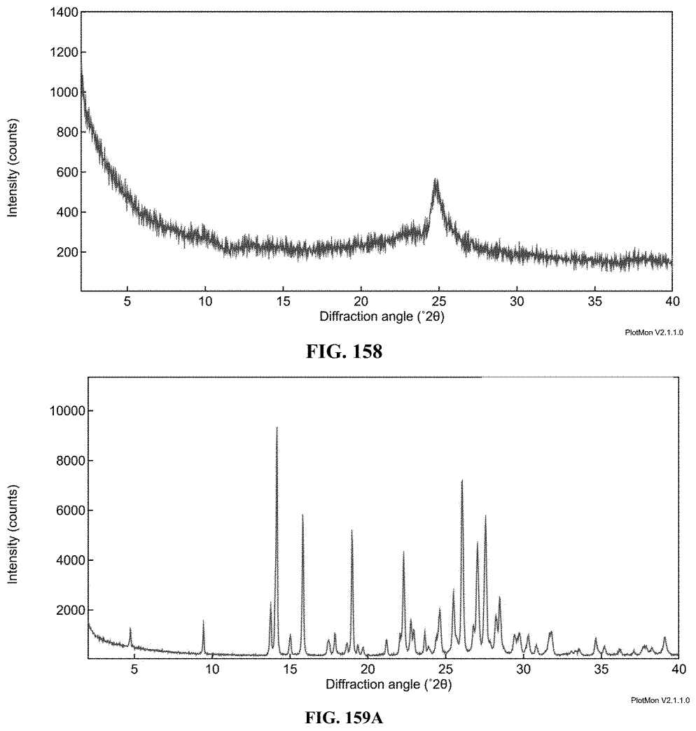

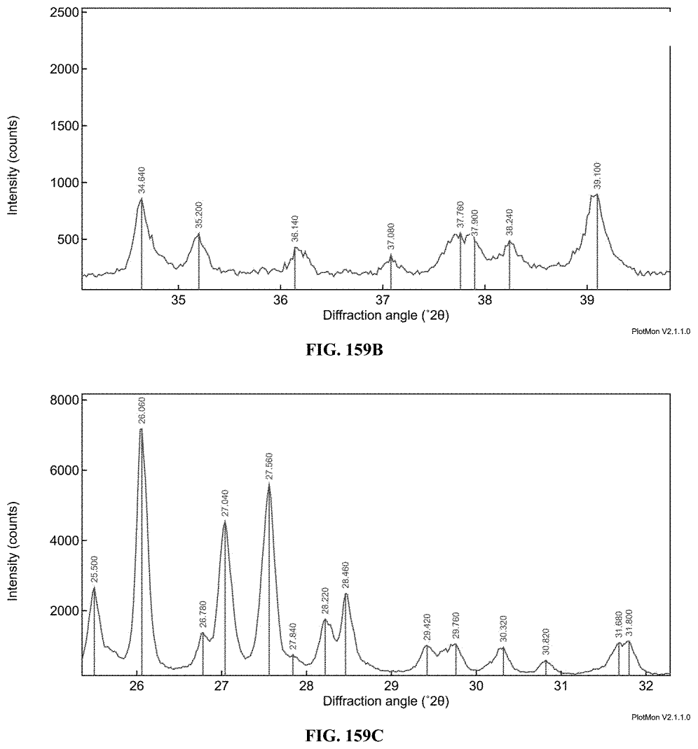

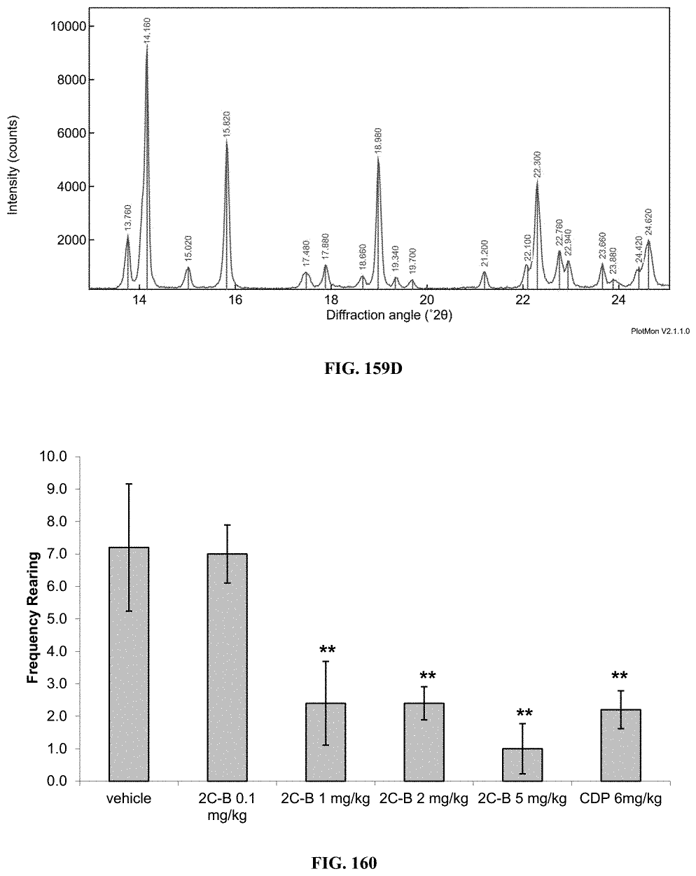

Characterization data is provided in FIGS. 135B, 146D and 153-158.

### Example 9—Crystallization and XRPD Analysis of Mescaline HCl

### Summary

A sample of 3,4,5-trimethoxyphenethylamine hydrochloride (Mescaline HCl) was crystallized and analyzed by X-ray powder diffraction (XRPD).

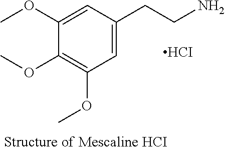

### Crystallization Experiments

One attempt was made to crystallize mescaline HCl. Details of that experiment are described in Table 90. The resulting material was crystalline.

TABLE 90
Crystallization Experiment.

| | FIG. | XRPD |
|---|---|---|
| Experimental Details | No. | Result |
| 50.4 mg of mescaline HCl was suspended in 1 mL | 159A-D | Crystalline |
| of EtOH. The slurry was stirred magnetically | | |
| on a hot plate set to 60° C. The solids dissolved. | | |
| The vial was removed from the plate and | | |
| allowed to cool to room temperature. Solids | | |
| were observed the next day. The sample was | | |
| centrifuged, the mother liquor decanted, and | | |
| the solids allowed to air dry. | | |

TABLE 91
XRPD Signal angle data of mescaline HCl

| Position (°2θ) | d-value | Relative |
|---|---|---|
| 4.7 | 18.64 | 14 |
| 9.4 | 9.37 | 15 |
| 13.8 | 6.44 | 23 |
| 14.2 | 6.25 | 100 |
| 15.0 | 5.90 | 11 |
| 15.8 | 5.60 | 62 |
| 17.5 | 5.07 | 9 |
| 17.9 | 4.96 | 12 |
| 18.7 | 4.76 | 7 |
| 19.0 | 4.68 | 55 |
| 19.3 | 4.59 | 7 |
| 19.7 | 4.51 | 6 |
| 21.2 | 4.19 | 9 |
| 22.1 | 4.02 | 11 |
| 22.3 | 3.99 | 45 |
| 22.8 | 3.91 | 17 |
| 22.9 | 3.88 | 13 |
| 23.7 | 3.76 | 12 |
| 23.9 | 3.73 | 6 |
| 24.4 | 3.65 | 11 |
| 24.6 | 3.62 | 22 |
| 25.5 | 3.49 | 29 |
| 26.1 | 3.42 | 79 |
| 26.8 | 3.33 | 15 |
| 27.0 | 3.30 | 50 |
| 27.6 | 3.24 | 61 |
| 27.8 | 3.20 | 8 |
| 28.2 | 3.16 | 19 |
| 28.5 | 3.14 | 27 |
| 29.4 | 3.04 | 11 |
| 29.8 | 3.00 | 12 |
| 30.3 | 2.95 | 11 |
| 30.8 | 2.90 | 6 |
| 31.7 | 2.82 | 12 |
| 31.8 | 2.81 | 12 |
| 33.1 | 2.71 | 4 |
| 33.3 | 2.69 | 4 |
| 33.5 | 2.67 | 5 |
| 34.6 | 2.59 | 9 |
| 35.2 | 2.55 | 6 |
| 36.1 | 2.49 | 5 |
| 37.1 | 2.42 | 4 |
| 37.8 | 2.38 | 6 |
| 37.9 | 2.37 | 5 |
| 38.2 | 2.35 | 5 |
| 39.1 | 2.30 | 10 |

### Experimental

### X-Ray Powder Diffraction (XRPD)

A Rigaku SmartLab X-Ray Diffractometer was configured in Bragg-Brentano reflection geometry equipped with a beam stop and knife edge to reduce incident beam and air scatter. Data collection parameters are shown in the following table. This method has not been validated.

PXRD Data Collection Parameters

| Parameter | Value | Parameter | Value |
|---|---|---|---|
| Geometry | Bragg-Brentano | Receiving Slit 1 (mm) | 18 |
| Tube Anode | Cu | Receiving Slit 2 (mm) | 20 |
| Tube Type | Long Fine Focus | Start Angle 2θ (°) | 2 |
| Tube Voltage (kV) | 40 | End Angle 2θ (°) | 40 |
| Tube Current (mA) | 45 | Step Size (°) | 0.02 |
| Detector | HyPix-3000 | Scan Speed (°/min) | 6 |
| Monochromator | Nifoil CuKβ Filter | Spinning (rpm) | 11 |
| Incident Slit (°) | ⅓ | Sample Holder | Low-background Si |

In some embodiments, the mescaline HCl is a crystalline polymorph characterized by two or more, or three XRPD signals selected from the group consisting of 9.4 °2θ, 13.8 °2θ, and 14.2 °2θ (±0.2 °2θ; ±0.1 °2θ; or ±0.0 °2θ; Cu Kα1 radiation).

In some embodiments, the mescaline HCl is a crystalline polymorph characterized by two or more, or three XRPD signals selected from the group consisting of 9.4 °2θ, 13.8 °2θ, 14.2 °2θ, 15.8 °2θ, and 19.0 °2θ (±0.2 °2θ; ±0.1 °2θ; or ±0.0 °2θ; Cu Kα1 radiation).

### Example 10—Zeromaze Study

### Background

The rat zero-maze model is a refined alternative to the plus-maze, the most widely used animal model of anxiety, and consists of an elevated annular platform, divided equally into four quadrants. Two opposite quadrants are enclosed by Perspex walls on both the inner and the outer edges of the platform, while the remaining two opposite quadrants are open being enclosed only by a Perspex “lip”. Animals will show a preference for the closed areas, and avoidance of the open sections is assumed to stem from a rodent's natural aversion to open, exposed spaces. A reduction in the amount of activity on the open areas is considered to reflect an increase in anxiety (shepherd 1994). The ethologically-based behavior rearing was also assessed as an index of anxiety with a decrease in rearing indicative of an anxiolytic effect [(a) File 1985, Boissier 1976; (b) File, S. E. (1985). What can be learned from the effects of benzodiazepines on exploratory behavior? Neuroscience & Biobehavioral Reviews, 9(1), 45-54. doi:10.1016/0149-7634(85)90031-4; (c) Boissier, J. R., P. Simon and P. Soubrie. New approaches to the study of anxiety and anxiolytic drugs in animals. In: Central Nervous System and Bheavioural Pharmacology, edited by M. Airaksinen. Oxford: Pergamon, 1976, pp. 213-222; and (d) Shepherd, J K; Grewel, S S; Fletcher, A; Bill, D J; Dourish, C T (1994) Behavioural and pharmacological validation of the elevated “zero-maze” as an animal model of anxiety. Psychopharmacol., 116:56-64.]

### Animals

Male Sprague-Dawley 200-250 g (Envigo UK) rats were used. Animals were group-housed (5 per cage; cage size: 40×40×20 cm) in a temperature-controlled environment (22±2° C.), under a 12 h light-dark cycle (lights on: 08:00 hours) for one week prior to testing. Food and water were freely available. Number of animals per group=5. Animals were moved into the experimental room 16-24 hours before testing.

### Apparatus

The elevated 0-maze comprises a black Perspex annular platform (105 cm diameter, 10 cm width) elevated to 65 cm above ground level, divided equally into four quadrants. Two opposite quadrants are enclosed by clear red Perspex walls (27 cm high) on both the inner and outer edges of the platform, while the remaining two opposite quadrants are surrounded only by a Perspex “lip” (1 cm high) which serves as a tactile guide to animals on these open areas.

### Procedure

Subjects were weighed and tail marked before being injected. After a specified pre-treatment time, subjects were placed in a closed quadrant and a 5-min test period were recorded on videotape for subsequent analysis. The maze was cleaned with 5% methanol/water solution and dried thoroughly between test sessions. Behavioral measures comprise percentage time spent on the open areas (% TO) and frequency of rearing. Animals are scored as being in the open area when all four paws were in an open quadrant and in the closed area only when all four paws have passed over the open-closed divide. All testing were carried out between 9.00 and 16.00 hours.

### Formulation:

IP: 2C-B was formulated in Vehicle 1 (Saline) for injection to concentrations of 0.02, 0.2, 0.4 and 1 mg/mL to provide doses of 0.1, 1, 2 and 5 mg/kg when administered ip in 5 mL/kg dosing volumes.

Chlordiazepoxide was formulated in Vehicle 1 (saline) to a concentration of 1.2 mg/mL to provide a dose of 6 mg/kg when administered ip in 5 mL/kg dosing volumes.

### Effect of Administration of 2C-B and Chlordiazepoxide on Behavior in a Rat 0-Maze Study

30 male Sprague-Dawley rats in treatment groups of 5, was intraperitoneally dosed with either Vehicle 1 (saline) or 2C-B at 1 of 4 dose levels (0.1, 1, 2 & 5 mg/kg) or chlordiazepoxide (6 mg/kg) in 5 mL/kg injection volumes. Thirty min later at T=0, rats were individually placed in a closed arm of the zero-maze and behavior was assessed by a “blind” observer using remote video monitoring over the subsequent 5 min. The animal were then removed and the maze carefully wiped with 5% methanol/water solution before the next test was begun.

### Statistical Analysis

Data was analysed with Statistica software (Statsoft USA version 10.0). All data was expressed as means±SEM. Data was analysed by 1 way ANOVA and Newman-Keuls test. Statistical significance in all analyses was assumed when P<0.05.

TABLE 92
Synopsis of testing schedule 2C-B and chlordiazepoxide
in the rat elevated zero maze model of anxiety.

| | Rat Strain | 0.5 Pretest in Veh 1 | T = 0 |
|---|---|---|---|
| n | & sex | 5 mL/kg ip | Zero-maze |
| 5 | Male SD | Vehicle 1 | Test |
| 5 | Male SD | 2C-B 0.1 mg/kg | Test |
| 5 | Male SD | 2C-B 1 mg/kg | Test |
| 5 | Male SD | 2C-B 2 mg/kg | Test |
| 5 | Male SD | 2C-B 5 mg/kg | Test |
| 5 | Male SD | CDP 6 mg/kg | Test |

### Results

The positive control CDP did not show significance over vehicle on the percentage of time in the open arms (% TO) measure due to underpowering so we could not use % TO in this experiment as a measure to examine the effects of 2C-B. However, on the measure of frequency of rearing 2C-B significantly reduced the number of rears in a dose dependent manner with 5 mg/kg having the greatest numerical effect. The ability of 2C-B to reduce rearing was as effective as the chlordiazepoxide control (FIG. 160).

### Discussion

The results show that the 1 mg/kg, 2 mg/kg, and 5 mg/kg doses of 2C-B, decreased the frequency of rearing as effectively as the benzodiazepine chlordiazepoxide (FIG. 160). This shows that 2C-B exhibits a dose dependent response on anxiety and that 2C-B is an effective anxiolytic and supports its development in these indications.

### Example 11—Effect of Orally Administered Dexamethasone and Intraperitoneally Administered 2C-B Hydrochloride in Rats on LPS-Induced TNF-α Release Ex Vivo

The bacterial endotoxin lipopolysaccharide (LPS) induces a strong inflammatory response from normal animal immune systems. This response is triggered when LPS is recognized by the innate immune system via the Toll Like Receptor (TLR4) particularly those located on monocytes and macrophages although they are also located on a wide variety of other cell types. Stimulation of the TLR4 receptor leads to recruitment and activation of IL-1R associated kinase 1 (IRAK-1) initiating a series of signalling events in immunoregulatory pathways such as activation of NF kappa B, p38 MAP Kinase, ERK and JNK and AP-1. These changes promote gene transcription and thereby potentiate secretion of pro inflammatory factors such as TNF alpha and other cytokines (Fang et al., 2004).

Determination of the effects of novel agents against LPS-induced TNF alpha release ex vivo provides an assessment of whether the plasma level of compound achieved after administration to whole animal is capable of affecting the inflammatory response.

### Formulation

### In Vivo Formulation

Dexamethasone disodium phosphate salt was formulated in water to a concentration of 0.6 mg free base/mL to give a dose of 3 mg free base/kg when orally administered in a 5 mL/kg dosing volume. 2C-B hydrochloride was formulated in Vehicle 1 (saline) at concentrations of 0.2 and 2 mg/mL. This provided doses of 0.1 and 1 mg/kg respectively when injected ip in 5 mL/kg dosing volumes. Ex vivo formulation

LPS were dissolved in saline to concentrations of 0.2 and 0.6 mg/mL. This will then be diluted 10 fold in saline to give concentrations of 0.02 and 0.06 mg/mL that will provide concentrations of 1 and 3 μg/mL when 15 uL is added to 285 of whole blood (20 fold dilution).

### Animals

Twenty four male SD rats (approximately 200 g), were group housed. Animals were maintained under a 12 h light/dark cycle, where temperature and humidity are controlled. Animals were allowed free access to food and water.

### Procedure

### Treatments and Blood Sampling

Rats were allocated to treatment groups of 6 and habituated to the test room on the day prior to the study. On the test day, rats were dosed orally with either water or dexamethasone 3 mg/kg in 5 mL/kg dosing volumes. Thirty minutes later the animals were dosed intraperitoneally with either saline or 2C-B in 5 mL/kg dosing volumes. After a further 90 minutes terminal blood samples were collected by cardiac puncture under CO₂.
Three aliquots, each of 285 μL from each animal's blood sample were pipetted into separate Eppendorf tubes.
Tubes were left for 10 min
Assay tubes will then be mixed gently before adding 15 μL of saline or LPS to give final concentrations of 1 and 3 μg/ml.
After a further gentle mixing, assay tubes were placed in an incubator at 37° C. for 24 h.
At the end of this time, the whole blood aliquots were spun down to plasma. The plasma were then removed and placed in duplicate 96 well plates on dry ice. Plates were stored at −20° C. until analyzed for TNF-α after 10-fold dilution.

### TNF-Alpha Measurement

The TNF-alpha content of the collected rat plasmas samples were measured using a Rat TNF-alpha Quantikine ELISA Kit (cat no RTA00).

### Statistical Analysis

### Individual 1-Way ANOVA

A 1-way ANOVA was conducted across the vehicle and 3 treatment groups (0.1 mg/kg 2C-B IP, 1 mg/kg 2C-B IP, and 3 mg/kg dexamethasone PO) independently for 3 stimulation conditions (no stimulation [saline], 1 μg/mL LPS, and 3 μg/mL LPS). This analysis assumed equal standard deviation and Gaussian distribution of data. Each group was compared to the vehicle group and a p-value for treatment determined by Fishers Least Significant Difference (LSD) test, without statistical penalty for multiple comparisons. This analysis was performed in GraphPad Prism (Version 9).

### 2-Way ANOVA

A 2-way ANOVA was conducted across the vehicle and 3 treatment groups (0.1 mg/kg 2C-B IP, 1 mg/kg 2C-B IP, and 3 mg/kg dexamethasone PO) for 3 stimulation conditions (no stimulation [saline], 1 μg/mL LPS, and 3 μg/mL LPS). This analysis assumed equal standard deviation and Gaussian distribution of data. There was a significant effect of both treatment and LPS simulation. Each treatment group was compared to the vehicle group and a p-value for treatment corrected for multiplicity by Dunnett's test was determined. This analysis was performed in GraphPad Prism (Version 9).

Total number or rats=24

Total number of whole blood samples for ex vivo TNF-alpha assessment=72.

TABLE 93
Synopsis of dexamethasone and Compound 1 vs LPS-induced TNF-alpha release ex vivo

| | In vivo phase | | | Ex vivo phase | | |
|---|---|---|---|---|---|---|
| | T = 0 | | T = 120 min | N | | |
| | Treatment po | | Terminal | Samples | Ex vivo | |
| | in Vehicle 1 | T = 30 min | plasma | for ex vivo | sample | Cytokine |
| n | (saline) | ip treatment | sampling | challenge | challenge | determination |
| 6 | Vehicle 1 | Saline | Yes | 6 | Saline | TNF-α |
| | | | | 6 | LPS 1 | TNF-α |
| | | | | | μg/mL | |
| | | | | 6 | LPS 3 | TNF-α |
| | | | | | μg/mL | |
| 6 | Vehicle 1 | 2C-B | Yes | 6 | Saline | TNF-α |
| | | hydrochloride | | 6 | LPS 1 | TNF-α |
| | | 0.1 mg/kg | | | μg/mL | |
| | | | | 6 | LPS 3 | TNF-α |
| | | | | | μg/mL | |
| 6 | Vehicle 1 | 2C-B | Yes | 6 | Saline | TNF-α |
| | | hydrochloride | | 6 | LPS 1 | TNF-α |
| | | 1 mg/kg | | | μg/mL | |
| | | | | 6 | LPS 3 | TNF-α |
| | | | | | μg/mL | |
| 6 | Dexamethasone | Saline | Yes | 6 | Saline | TNF-α |
| | 3 mg/kg | | | 6 | LPS 1 | TNF-α |
| | | | | | μg/mL | |
| | | | | 6 | LPS 3 | TNF-α |
| | | | | | μg/mL | |

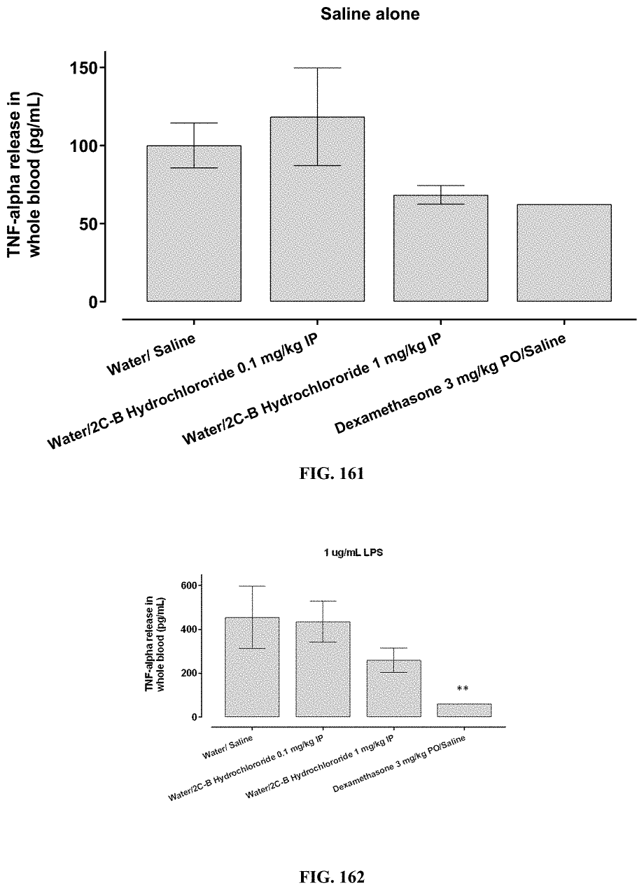

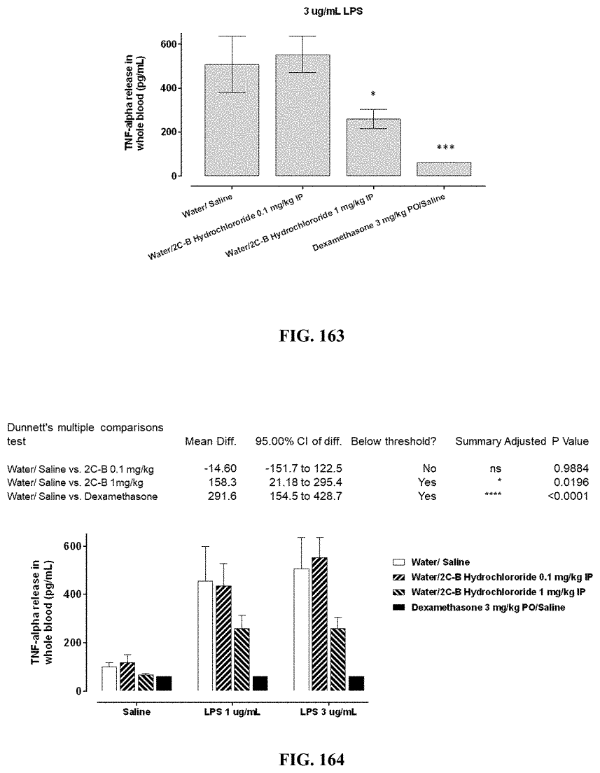

We tested the ability of two dose levels of 2C-B to reduce TNF alpha levels in response to a low and high dose of LPS. As a control, we found that there was no effect of 2C-B, saline, or dexamethasone on TNF alpha levels in the absence of LPS (FIG. 161). When LPS was administered we found that saline did not significantly reduce the level of TNF-alpha release in response to LPS. As expected, the positive control the steroid dexamethasone significantly reduced the level of TNF alpha. The data shows with the LPS low dose of 1 2C-B dose dependently reduce the total levels of TNF alpha with 0.1 mg/kg of 2C-B having no difference vs placebo and an effect level that trended towards significance with 1 mg/kg of 2C-B (FIG. 162). With the high level of LPS of 3 μg/ml, 2C-B significantly reduced the level of TNF alpha in a dose dependent manner with 1 mg/kg resulting in a significant decrease in TNF alpha levels (FIG. 163). Finally we analyzed the data again using a 2-way ANOVA. Each treatment group was compared to the vehicle group and a p-value for treatment corrected for multiplicity by Dunnett's test was determined. Similar to the other comparisons, 2C-B again showed a significant effect (P=0.0196) on the high level of LPS of 3 μg/ml (FIG. 164).

### Discussion

We found that pre-treatment with 2C-B in vivo reduced the amount of TNF alpha released in response to LPS (FIGS. 161-164). This is surprising since 2C-B is not a steroid like dexamethasone. This result shows that 2C-B is a highly effective anti-inflammatory with a completely different structural class and likely different mechanistic class. Because 2C-B is not known to have the severe side effects caused by steroids, this may represent a new treatment for inflammation with a safer side effect profile than steroids including dexamethasone.

In view of the many possible embodiments to which the principles of the disclosed invention may be applied, it should be recognized that the illustrated embodiments are only preferred examples of the invention and should not be taken as limiting the scope of the invention. Rather, the scope of the invention is defined by the following claims. We therefore claim as our invention all that comes within the scope and spirit of these claims.

1. A salt or solid form of 4-bromo-2,5-dimethoxyphenethylamine (2C-B).

2. The salt or solid form of 2C-B of claim 1, wherein the salt is crystalline or amorphous.

3. (canceled)

4. The salt of 2C-B of claim 1, wherein the salt is 2C-B HCl, 2C-B Besylate, 2C-B Citrate, 2C-B Esylate, 2C-B Fumarate, 2C-B Gentisate, 2C-B Gluconate, 2C-B Glycolate, 2C-B Sulfate, 2C-B Phosphate, 2C-B Xinafoate, 2C-B Lactate, 2C-B Malate, 2C-B Maleate, 2C-B Malonate, 2C-B Mesylate, 2C-B Mucate, 2C-B Succinate, 2C-B Tartrate, 2C-B Tosylate, 2C-B Aspartate, or 2C-B Glutamate.

5. The 2C-B-HCl.salt of claim 4, wherein the salt is crystalline polymorphic form selected from;
Form A, which is characterized by at least one of the characteristics selected from:
(a) two or more, or three XRPD signals selected from the group consisting of 5.7 °2θ, 24.1 °2θ, and 23.4 °2θ (±0.2 °2θ; ±0.1 °2θ; or ±0.0 °2θ; Cu Kα1 radiation);
(b) two, three, four, five, six, seven, eight, nine, or ten XRPD signals as shown in Table 6;
(c) two or more, or three XRPD signals as shown in FIG. 1;
(d) by two or more, or three XRPD signals selected from the group consisting of 24 °2θ, 23.4 °2θ, and 5.6 °2θ (±0.2 °2θ; ±0.1 °2θ; or 0.0 °2θ; Cu Kα1 radiation);
(e) two, three, four, five, six, seven, eight, nine, or ten XRPD signals as shown in Table 7;
(f) two or more, or three XRPD signals as shown in FIG. 2;
(g) two or more, or three XRPD signals selected from the group consisting of 5.6 °2θ, 16.9 °2θ, and 34.2 °2θ (±0.2 °2θ; ±0.1 °2θ; or 0.0 °2θ; Cu Kα1 radiation);
(h) two, three, four, five, six, seven, eight, nine, or ten XRPD signals as shown in Table 12;
(i) two or more, or three XRPD signals as shown in FIG. 7;
(j) a DVS profile as provided in FIG. 53;
(k) two or more, or three XRPD signals selected from the group consisting of 5.7 °2θ, 16.9 °2θ, and 17.4 °2θ (±0.2 °2θ; ±0.1 °2θ; or 0.0 °2θ; Cu Kα1 radiation);
(l) two, three, four, five, six, seven, eight, nine, or ten XRPD signals as shown in Table 58;
(m) two or more, or three XRPD signals selected from the group consisting of 5.6 °2θ, 11.3 °2θ, and 16.9 °2θ (±0.2 °2θ; ±0.1 °2θ; or +0.0 °2θ; Cu Kα1 radiation);
(n) two or more, or three XRPD signals selected from the group consisting of 5.6 °2θ, 11.3 °2θ, 16.9 °2θ, 22.6 °2θ and 28.4 °2θ (±0.2 °2θ; ±0.1 °2θ; or 0.0 °2θ; Cu Kα1 radiation);
(o) two or more, or three XRPD signals selected from the group consisting of 5.6 °2θ, 16.8 °2θ, and 17.4 °2θ (±0.2 °2θ; ±0.1 °2θ; or 0.0 °2θ; Cu Kα1 radiation);
(p) two or more, or three XRPD signals selected from the group consisting of 5.6 °2θ, 16.8 °2θ, and 17.4 °2θ, 16.2 °2θ and 24 °2θ (±0.2 °2θ; ±0.1 °2θ; or 0.0 °2θ; Cu Kα1 radiation); and
(q) two or more, or three XRPD signals selected from the group consisting of 5.7 °2θ, 16.9 °2θ, and 17.4 °2θ, 16.2 °2θ and 24.1 °2θ (±0.2 °2θ; ±0.1 °2θ; or +0.0 °2θ; Cu Kα1 radiation);
Form B, which is characterized by at least one of the characteristics selected from;
(a) two or more, or three XRPD signals selected from the group consisting of 5.5 °2θ, 16.6 °2θ, and 30.2 °2θ (±0.2 °2θ; ±0.1 °2θ; or ±0.0 °2θ; Cu Kα1 radiation);
(b) two, three, four, five, six, seven, eight, nine, or ten XRPD signals as shown in Table 8;
(c) two or more, or three XRPD signals as shown in FIG. 3;
(d) an ¹H NMR spectra as provided in FIG. 46;
(e) a TGA and DSC profiles as provided in FIG. 51;
(f) two or more, or three XRPD signals selected from the group consisting of 5.6 °2θ, 16.2 °2θ, and 16.6 °2θ (±0.2 °2θ; ±0.1 °2θ; or 0.0 °2θ; Cu Kα1 radiation);
(g) two or more, or three XRPD signals selected from the group consisting of 23.6 °2θ, 5.6 °2θ, and 30.3 °2θ (±0.2 °2θ; ±0.1 °2θ; or ±0.0 °2θ; Cu Kα1 radiation);
(h) two, three, four, five, six, seven, eight, nine, or ten XRPD signals as shown in Table 57;
(i) two or more, or three XRPD signals selected from the group consisting of 5.5 °2θ, 16.0 °2θ, and 16.6 °2θ (±0.2 °2θ; ±0.1 °2θ; or 0.0 °2θ; Cu Kα1 radiation); and
(j) two or more, or three XRPD signals selected from the group consisting of 5.5 °2θ, 16.0 °2θ, 16.6 °2θ, 23.4 °2θ, and 30.2 °2θ (±0.2 °2θ; ±0.1 °2θ; or ±0.0 °2θ; Cu Kα1 radiation); and
Form C, which is characterized by at least one of the characteristics selected from;
(a) two or more, or three XRPD signals selected from the group consisting of 4.5 °2θ, 25.9 °2θ, and 34.4 °2θ (±0.2 °2θ; ±0.1 °2θ; or ±0.0 °2θ; Cu Kα1 radiation);
(b) two, three, four, five, six, seven, eight, nine, or ten XRPD signals as shown in Table 9;
(c) two or more, or three XRPD signals as shown in FIG. 4;
(d) an ¹H NMR spectra as provided in FIG. 47;
(e) TGA and DSC profiles as provided in FIG. 52;
(f) two or more, or three XRPD signals selected from the group consisting of 4.5 °2θ, 9.0 °2θ, and 13.6 °2θ (±0.2 °2θ; ±0.1 °2θ; or ±0.0 °2θ; Cu Kα1 radiation); and
(g) two or more, or three XRPD signals selected from the group consisting of 4.5 °2θ, 9.0 °2θ, 13.6 °2θ, 18.3 °2θ, and 25.9 °2θ (±0.2 °2θ; ±0.1 °2θ; or ±0.0 °2θ; Cu Kα1 radiation).

6.-22. (canceled)

23. The 2C-B besylate salt of claim 4, wherein the salt is crystalline polymorphic form selected from;
Form 1, which is characterized by at least one of the characteristics selected from;
(a) two or more, or three XRPD signals selected from the group consisting of 13.4 °2θ, 24.4 °2θ, and 25.7 °2θ (±0.2 °2θ; ±0.1 °2θ; or ±0.0 °2θ; Cu Kα1 radiation);
(b) an ¹H NMR spectra as provided in FIG. 73;
(c) an ¹H NMR spectra as provided in FIG. 98;
(d) TGA and DSC profiles as provided in FIG. 107;
(e) TGA and DSC profiles as provided in FIG. 125;
(f) a DVS profile as provided in FIG. 137;
(g) by two or more, or three XRPD signals selected from the group consisting of 8.4 °2θ, 11.5 °2θ, 13.4 °2θ, 15.5 °2θ, and 16.7 °2θ (±0.2 °2θ; ±0.1 °2θ; or 0.0 °2θ Cu Kα1 radiation);
(h) two or more, or three XRPD signals selected from the group consisting of 8.4 °2θ, 12.1 °2θ, and 13.4 °2θ (±0.2 °2θ; ±0.1 °2θ; or ±0.0 °2θ; Cu Kα1 radiation);
(i) two or more, or three XRPD signals selected from the group consisting of 8.4 °2θ, 12.1 °2θ, 13.4 °2θ, 15.5 °2θ, and 16.7 °2θ (±0.2 °2θ; ±0.1 °2θ; or ±0.0 °2θ; Cu Kα1 radiation); and
(j) two or more, or three XRPD signals selected from the group consisting of 8.4 °2θ, 11.5 °2θ, and 13.4 °2θ (±0.2 °2θ; ±0.1 °2θ; or ±0.0 °2θ; Cu Kα1 radiation); and
Form 2, which is characterized by at least one of the characteristics selected from;
(a) two or more, or three XRPD signals selected from the group consisting of 20 °2θ, 19 °2θ, and 24.1 °2θ (±0.2 °2θ; ±0.1 °2θ; or 0.0 °2θ; Cu Kα1 radiation);
(b) an ¹H NMR spectra as provided in FIG. 74;
(c) TGA and DSC profiles as provided in FIG. 108;
(d) two or more, or three XRPD signals selected from the group consisting of 5.7 °2θ, 11.4 °2θ, and 19.0 °2θ (±0.2 °2θ; ±0.1 °2θ; or ±0.0 °2θ; Cu Kα1 radiation); and
(e) two or more, or three XRPD signals selected from the group consisting of 5.7 °2θ, 11.4 °2θ, 19.0 °2θ, 16.4 °2θ, and 20.0 °2θ (±0.2 °2θ; ±0.1 °2θ; or ±0.0 °2θ; Cu Kα1 radiation).

24.-26. (canceled)

27. The 2C-B citrate salt of claim 4, wherein the salt is crystalline polymorphic form selected from;
Form 1, which is characterized by at least one of the characteristics selected from;
(a) two or more, or three XRPD signals selected from the group consisting of 24.7 °2θ, 22.5 °2θ, and 13.7 °2θ (±0.2 °2θ; ±0.1 °2θ; or 0.0 °2θ; Cu Kα1 radiation);
(b) an ¹H NMR spectra as provided in FIG. 75;
(c) TGA and DSC profiles as provided in FIG. 109;
(d) two or more, or three XRPD signals selected from the group consisting of 11.0 °2θ, 11.2 °2θ, and 12.8 °2θ (±0.2 °2θ; ±0.1 °2θ; or ±0.0 °2θ; Cu Kα1 radiation); and
(e) two or more, or three XRPD signals selected from the group consisting of 11.0 °2θ, 11.2 °2θ, and 12.8 °2θ, 13.7 °2θ, and 14.2 °2θ (±0.2 °2θ; ±0.1 °2θ; or 0.0 °2θ; Cu Kα1 radiation); and
Form 2, which is characterized by at least one of the characteristics selected from;
(a) two or more, or three XRPD signals selected from the group consisting of 20.6 °2θ, 21.8 °2θ, and 16.6 °2θ (±0.2 °2θ; ±0.1 °2θ; or ±0.0 °2θ; Cu Kα1 radiation);
(b) an ¹H NMR spectra as provided in FIG. 99;
(c) TGA and DSC profiles as provided in FIG. 126;
(d) a DVS profile as provided in FIG. 138;
(e) two or more, or three XRPD signals selected from the group consisting of 3.7 °2θ, 10.3 °2θ, and 13.1 °2θ (±0.2 °2θ; ±0.1 °2θ; or ±0.0 °2θ; Cu Kα1 radiation); and
(f) two or more, or three XRPD signals selected from the group consisting of 3.7 °2θ, 10.3 °2θ, 13.1 °2θ, 14.5 °2θ, and 16.6 °2θ (±0.2 °2θ; ±0.1 °2θ; or 0.0 °2θ; Cu Kα1 radiation).

28-30. (canceled)

31. The 2C-B esylate salt of claim 4, wherein the salt is crystalline polymorphic form selected from;
Form 1, which is characterized by at least one of the characteristics selected from;
(a) two or more, or three XRPD signals selected from the group consisting of 9.3 °2θ, 23.6 °2θ, and 23.7 °2θ (±0.2 °2θ; ±0.1 °2θ; or 0.0 °2θ; Cu Kα1 radiation);
(b) an ¹H NMR spectra as provided in FIG. 76;
(c) TGA and DSC profiles as provided in FIG. 110;
(d) two or more, or three XRPD signals selected from the group consisting of 9.3 °2θ, 10.0 °2θ, and 14.5 °2θ (±0.2 °2θ; ±0.1 °2θ; or ±0.0 °2θ; Cu Kα1 radiation); and
(e) two or more, or three XRPD signals selected from the group consisting of 9.3 °2θ, 10.0 °2θ, and 14.5 °2θ, 17.4 °2θ, and 18.7 °2θ (±0.2 °2θ; ±0.1 °2θ; or 0.0 °2θ; Cu Kα1 radiation); and
Form 2, which is characterized by at least one of the characteristics selected from;
(a) two or more, or three XRPD signals selected from the group consisting of 24.1 °2θ, 13.9 °2θ, and 22.7 °2θ (±0.2 °2θ, ±0.1 °2θ; or ±0.0 °2θ, Cu Kα1 radiation);
(b) an ¹H NMR spectra as provided in FIG. 77;
(c) TGA and DSC profiles as provided in FIG. 111;
(d) two or more, or three XRPD signals selected from the group consisting of 8.6 °2θ, 12.5 °2θ, and 13.9 °2θ (±0.2 °2θ; ±0.1 °2θ; or 0.0 °2θ; Cu Kα1 radiation); and
(e) two or more, or three XRPD signals selected from the group consisting of 8.6 °2θ, 12.5 °2θ, and 13.9 °2θ, 15.2 °2θ, and 17.5 °2θ (±0.2 °2θ; ±0.1 °2θ; or 0.0 °2θ; Cu Kα1 radiation).

32.-34. (canceled)

35. The 2C-B fumarate salt of claim 4, wherein the salt is crystalline polymorphic (Form 1) characterized by at least one of the characteristics selected from;
(a) two or more, or three XRPD signals selected from the group consisting of 22.9 °2θ, 14.6 °2θ, and 21.4 °2θ (±0.2 °2θ; ±0.1 °2θ; or 0.0 °2θ; Cu Kα1 radiation);
(b) an ¹H NMR spectra as provided in FIG. 78;
(c) TGA and DSC profiles as provided in FIG. 112;
(d) two or more, or three XRPD signals selected from the group consisting of 7.1 °2θ, 14.6 °2θ, and 15.6 °2θ (±0.2 °2θ: 0.1 °2θ; or 0.0 °2θ; Cu Kα1 radiation); and
(e) two or more, or three XRPD signals selected from the group consisting of 7.1 °2θ, 14.6 °2θ, 15.6 °2θ, 21.4 °2θ, and 22.9 °2θ (±0.2 °2θ; 0.1 °2θ; or 0.0 °2θ; Cu Kα1 radiation).

36.-37. (canceled)

38. The 2C-B gentisate salt of claim 4, wherein the salt is a crystalline polymorph, which is characterized by at least one of the characteristics selected from;
(a) two, three, four, five, six, seven, eight, nine, or ten XRPD signals as shown in Table 23;
(b) two or more, or three XRPD signals as shown in FIG. 12;
(c) two or more, or three XRPD signals selected from the group consisting of 22.2 °2θ, 25.6 °2θ, and 13.1 °2θ (±0.2 °2θ; 0.1 °2θ; or ±0.0 °2θ; Cu Kα1 radiation);
(d) two, three, four, five, six, seven, eight, nine, or ten XRPD signals as shown in Table 62;
(e) two or more, or three XRPD signals as shown in FIG. 56;
(f) an 1H NMR spectra as provided in FIG. 79;
(g) an 1H NMR spectra as provided in FIG. 100;
(h) TGA and DSC profiles as provided in FIG. 113;
(i) TGA and DSC profiles as provided in FIG. 127;
(j) a DVS profile as provided in FIG. 139;
(k) two or more, or three XRPD signals selected from the group consisting of 10.1 °2θ, 25.6 °2θ, and 22.2 °2θ (±0.2 °2θ; ±0.1 °2θ; or 0.0 °2θ; Cu Kα1 radiation);
(l) two, three, four, five, six, seven, eight, nine, or ten XRPD signals as shown in Table 63;
(m) two or more, or three XRPD signals as shown in FIG. 57;
(n) exhibit high crystallinity;
(o) exhibit acceptable scale-up;
(p) exhibit minimal water uptake over RH range;
(q) exhibit uncomplicated DSC data (one endothermic event observed);
(r) acceptable solubility when compared to the other salts;
(s) two or more, or three XRPD signals selected from the group consisting of 10.1 °2θ, 13.1 °2θ, and 16.5 °2θ (±0.2 °2θ; ±0.1 °2θ; or 0.0 °2θ; Cu Kα1 radiation);
(t) two or more, or three XRPD signals selected from the group consisting of 10.1 °2θ, 13.1 °2θ, 16.5 °2θ, 17.2 °2θ, and 22.2 °2θ (±0.2 °2θ; ±0.1 °2θ; or 0.0 °2θ; Cu Kα1 radiation);
(u) two or more, or three XRPD signals selected from the group consisting of 10.1 °2θ, 13.1 °2θ, 16.5 °2θ, 17.3 °2θ, and 20.2 °2θ (±0.2 °2θ; ±0.1 °2θ; or ±0.0 °2θ; Cu Kα1 radiation); and
(v) possess at least one of characteristic as provided in Table 86.

39.-49. (canceled)

50. The 2C-B glycolate salt of claim 4, wherein the salt is a crystalline polymorph, which is characterized by at least one of the characteristics selected from;
(a) two or more, or three XRPD signals selected from the group consisting of 26.4 °2θ, 19 °2θ, and 21.5 °2θ (±0.2 °2θ; ±0.1 °2θ; or ±0.0 °2θ; Cu Kα1 radiation);
(b) two, three, four, five, six, seven, eight, nine, or ten XRPD signals as shown in Table 25;
(c) two or more, or three XRPD signals as shown in FIG. 14;
(d) two or more, or three XRPD signals selected from the group consisting of 26.3 °2θ, 21.5 °2θ, and 19 °2θ (±0.2 °2θ; ±0.1 °2θ; or 0.0 °2θ; Cu Kα1 radiation);
(e) two, three, four, five, six, seven, eight, nine, or ten XRPD signals as shown in Table 65;
(f) two or more, or three XRPD signals as shown in FIG. 59;
(g) an ¹H NMR spectra as provided in FIG. 81;
(h) an ¹H NMR spectra as provided in FIG. 101;
(i) TGA and DSC profiles as provided in FIG. 115;
(j) TGA and DSC profiles as provided in FIG. 128;
(k) a DVS profile as provided in FIG. 140;
(l) exhibit high crystallinity;
(m) exhibit acceptable scale-up;
(n) exhibit minimal water uptake over RH range;
(o) exhibit uncomplicated DSC data (one endothermic event observed);
(p) acceptable solubility when compared to the other salts;
(q) possess at least one of characteristic as provided in Table 86;
(r) two or more, or three XRPD signals selected from the group consisting of 6.4 °2θ, 15.7 °2θ, and 16.1 °2θ (±0.2 °2θ; ±0.1 °2θ; or ±0.0 °2θ; Cu Kα1 radiation);
(s) two or more, or three XRPD signals selected from the group consisting of 6.4 °2θ, 15.7 °2θ, 16.1 °2θ, 19.0 °2θ, and 21.5 °2θ (±0.2 °2θ; ±0.1 °2θ; or 0.0 °2θ; Cu Kα1 radiation);
(t) two or more, or three XRPD signals selected from the group consisting of 6.3 °2θ, 15.6 °2θ, and 16 °2θ (±0.2 °2θ, ±0.1 °2θ; or ±0.0 °2θ, Cu Kα1 radiation); and
(u) two or more, or three XRPD signals selected from the group consisting of 6.3 °2θ, 15.6 °2θ, 16 °2θ, 19 °2θ, and 21.5 °2θ (±0.2 °2θ; ±0.1 °2θ; or 0.0 °2θ; Cu Kα1 radiation).

51.-58. (canceled)

59. The 2C-B sulfate salt of claim 4, wherein the salt is a crystalline polymorph, which is characterized by at least one of the characteristics selected from;
(a) two or more, or three XRPD signals selected from the group consisting of 25.1 °2θ, 25.7 °2θ, and 23.2 °2θ (±0.2 °2θ; ±0.1 °2θ; or 0.0 °2θ, Cu Kα1 radiation);
(b) two, three, four, five, six, seven, eight, nine, or ten XRPD signals as shown in Table 26;
(c) by two or more, or three XRPD signals as shown in FIG. 15;
(d) two or more, or three XRPD signals selected from the group consisting of 25.1 °2θ, 8.7 °2θ, and 23.2 °2θ (±0.2 °2θ; ±0.1 °2θ; or 0.0 °2θ; Cu Kα1 radiation);
(e) two, three, four, five, six, seven, eight, nine, or ten XRPD signals as shown in Table 68;
(f) two or more, or three XRPD signals as shown in FIG. 62;
(g) an ¹H NMR spectra as provided in FIG. 92;
(h) an ¹H NMR spectra as provided in FIG. 104;
(i) TGA and DSC profiles as provided in FIG. 121;
(j) TGA and DSC profiles as provided in FIG. 131;
(k) a DVS profile as provided in FIG. 143;
(l) exhibit high crystallinity;
(m) exhibit acceptable scale-up;
(n) exhibit minimal water uptake over RH range;
(o) exhibit uncomplicated DSC data (one endothermic event observed);
(p) acceptable solubility when compared to the other salts;
(q) possess at least one of characteristic as provided in Table 86;
(r) two or more, or three XRPD signals selected from the group consisting of 8.7 °2θ, 13.0 °2θ, and 16.4 °2θ (±0.2 °2θ; ±0.1 °2θ; or ±0.0 °2θ; Cu Kα1 radiation); and
(s) two or more, or three XRPD signals selected from the group consisting of 8.7 °2θ, 13.0 °2θ, 16.4 °2θ, 15.1 °2θ and 17.9 °2θ (±0.2 °2θ; ±0.1 °2θ; or ±0.0 °2θ; Cu Kα1 radiation).

60.-67. (canceled)

68. The 2C-B phosphate salt of claim 4, wherein the salt is crystalline polymorphic form selected from;
Form 1 and Form 2, which is characterized by at least one of the characteristics selected from;
(a) two or more, or three XRPD signals selected from the group consisting of 23.9 °2θ, 18.5 °2θ, and 25.3 °2θ (±0.2 °2θ; ±0.1 °2θ; or 0.0 °2θ; Cu Kα1 radiation);
(b) two or more, or three XRPD signals selected from the group consisting of 4.3 °2θ, 13.9 °2θ, and 15.7 °2θ (±0.2 °2θ; ±0.1 °2θ; or 0.0 °2θ; Cu Kα1 radiation);
(c) two or more, or three XRPD signals selected from the group consisting of 4.3 °2θ, 13.9 °2θ, 15.7 °2θ, 18.5 °2θ, and 18.8 °2θ (±0.2 °2θ, ±0.1 °2θ; or ±0.0 °2θ; Cu Kα1 radiation);
Form 1, which is characterized by at least one of the characteristics selected from;
(a) an ¹H NMR spectra as provided in FIG. 89;
(b) TGA and DSC profiles as provided in FIG. 119; and
Form 2, which is characterized by at least one of the characteristics selected from;
(a) two or more, or three XRPD signals selected from the group consisting of 24.8 °2θ, 8.6 °2θ, and 21.5 °2θ (±0.2 °2θ; ±0.1 °2θ; or 0.0 °2θ; Cu Kα1 radiation);
(b) an ¹H NMR spectra as provided in FIG. 90;
(c) two or more, or three XRPD signals selected from the group consisting of 8.6 °2θ, 12.9 °2θ, and 17.7 °2θ (±0.2 °2θ; 0.1 °2θ; or ±0.0 °2θ; Cu Kα1 radiation); and
(d) two or more, or three XRPD signals selected from the group consisting of 8.6 °2θ, 12.9 °2θ, and 17.7 °2θ, 21.5 °2θ, and 24.8 °2θ (±0.2 °2θ; 0.1 °2θ; or ±0.0 °2θ; Cu Kα1 radiation).

69.-71. (canceled)

72. The 2C-B xinafoate salt of claim 4, wherein the salt is a crystalline polymorph, which is characterized by at least one of the characteristics selected from;
(a) two or more, or three XRPD signals selected from the group consisting of 24.7 °2θ, 23.3 °2θ, and 18.5 °2θ (±0.2 °2θ; ±0.1 °2θ; or ±0.0 °2θ; Cu Kα1 radiation);
(b) two or more, or three XRPD signals selected from the group consisting of 24.8 °2θ, 23.3 °2θ, and 18.6 °2θ (±0.2 °2θ; ±0.1 °2θ; or ±0.0 °2θ; Cu Kα1 radiation);
(c) an ¹H NMR spectra as provided in FIG. 95;
(d) TGA and DSC profiles as provided in FIG. 124;
(e) two or more, or three XRPD signals selected from the group consisting of 7.9 °2θ, 13.9 °2θ, and 14.4 °2θ (±0.2 °2θ; 0.1 °2θ; or 0.0 °2θ; Cu Kα1 radiation);
(f) two or more, or three XRPD signals selected from the group consisting of 7.9 °2θ, 13.9 °2θ, 14.4 °2θ, 13.1 °2θ, and 18.6 °2θ (±0.2 °2θ; ±0.1 °2θ; or 0.0 °2θ; Cu Kα1 radiation);
(g) two or more, or three XRPD signals selected from the group consisting of 7.9 °2θ, 13.8 °2θ, and 14.3 °2θ (±0.2 °2θ; 0.1 °2θ; or ±0.0 °2θ; Cu Kα1 radiation); and
(h) two or more, or three XRPD signals selected from the group consisting of 7.9 °2θ, 13.8 °2θ, 14.3 °2θ, 13.0 °2θ, and 18.5 °2θ (±0.2 °2θ; 0.1 °2θ; or 0.0 °2θ; Cu Kα1 radiation).

73.-74. (canceled)

75. The 2C-B lactate salt of claim 4, wherein the salt is crystalline polymorphic form selected from;
Form 1, which is characterized by at least one of the characteristics selected from;
(a) two or more, or three XRPD signals selected from the group consisting of 7.1 °2θ, 23.2 °2θ, and 25.3 °2θ (±0.2 °2θ; ±0.1 °2θ; or ±0.0 °2θ; Cu Kα1 radiation);
(b) an ¹H NMR spectra as provided in FIG. 82;
(c) two or more, or three XRPD signals selected from the group consisting of 7.1 °2θ, 8.1 °2θ, and 10.7 °2θ (±0.2 °2θ; 0.1 °2θ; or ±0.0 °2θ, Cu Kα1 radiation);
(d) two or more, or three XRPD signals selected from the group consisting of 7.1 °2θ, 8.1 °2θ, 10.7 °2θ, 15.3 °2θ, and 23.2 °2θ (±0.2 °2θ; 0.1 °2θ; or ±0.0 °2θ; Cu Kα1 radiation); and
Form 2, which is characterized by at least one of the characteristics selected from;
(a) two or more, or three XRPD signals selected from the group consisting of 19.4 °2θ, 25.2 °2θ, and 10.5 °2θ (±0.2 °2θ; ±0.1 °2θ; or ±0.0 °2θ; Cu Kα1 radiation);
(b) an ¹H NMR spectra as provided in FIG. 83;
(c) two or more, or three XRPD signals selected from the group consisting of 5.3 °2θ, 7.4 °2θ, and 10.5 °2θ (±0.2 °2θ; 0.1 °2θ; or ±0.0 °2θ; Cu Kα1 radiation); and
(d) two or more, or three XRPD signals selected from the group consisting of 5.3 °2θ, 7.4 °2θ, 10.5 °2θ, 15.5 °2θ, and 19.4 °2θ (±0.2 °2θ; 0.1 °2θ, or ±0.0 °2θ; Cu Kα1 radiation).

76.-78. (canceled)

79. The 2C-B malate salt of claim 4, wherein the salt is a crystalline polymorph, which is characterized by at least one of the characteristics selected from;
(a) two or more, or three XRPD signals selected from the group consisting of 23.9 °2θ, 24.4 °2θ, and 19.8 °2θ (±0.2 °2θ; ±0.1 °2θ; or ±0.0 °2θ; Cu Kα1 radiation);
(b) two, three, four, five, six, seven, eight, nine, or ten XRPD signals as shown in Table 66;
(c) two or more, or three XRPD signals as shown in FIG. 60;
(d) an ¹H NMR spectra as provided in FIG. 84;
(e) an ¹H NMR spectra as provided in FIG. 102;
(f) TGA and DSC profiles as provided in FIG. 116;
(g) TGA and DSC profiles as provided in FIG. 129;
(h) a DVS profile as provided in FIG. 141;
(i) exhibit high crystallinity;
(j) exhibit acceptable scale-up;
(k) exhibit minimal water uptake over RH range;
(l) exhibit uncomplicated DSC data (one endothermic event observed);
(m) acceptable solubility when compared to the other salts;
(n) possesses at least one of characteristic as provided in Table 86;
(o) two or more, or three XRPD signals selected from the group consisting of 12.0 °2θ, 14.6 °2θ, and 16.5 °2θ (±0.2 °2θ; ±0.1 °2θ; or 0.0 °2θ; Cu Kα1 radiation); and
(p) two or more, or three XRPD signals selected from the group consisting of 12.0 °2θ, 14.6 °2θ, 16.5 °2θ, 17.9 °2θ, and 19.8 °2θ (±0.2 °2θ; ±0.1 °2θ; or ±0.0 °2θ; Cu Kα1 radiation).

80.-84. (canceled)

85. The 2C-B maleate salt of claim 4, wherein the salt is crystalline polymorphic form selected from;
Form 1, which is characterized by at least one of the characteristics selected from;
(a) two or more, or three XRPD signals selected from the group consisting of 21.7 °2θ, 25.1 °2θ, and 22.9 °2θ (±0.2 °2θ; ±0.1 °2θ; or ±0.0 °2θ; Cu Kα1 radiation);
(b) an ¹H NMR spectra as provided in FIG. 85;
(c) TGA and DSC profiles as provided in FIG. 117;
(d) two or more, or three XRPD signals selected from the group consisting of 5.7 °2θ, 10.7 °2θ, and 11.2 °2θ (±0.2 °2θ; ±0.1 °2θ; or ±0.0 °2θ; Cu Kα1 radiation); and
(e) two or more, or three XRPD signals selected from the group consisting of 5.7 °2θ, 10.7 °2θ, 11.2 °2θ, 9.3 °2θ, and 14.8 °2θ (±0.2 °2θ; ±0.1 °2θ; or 0.0 °2θ; Cu Kα1 radiation); and
Form 2, which is characterized by at least one of the characteristics selected from;
(a) two or more, or three XRPD signals selected from the group consisting of 25.9 °2θ, 26.2 °2θ, and 25.6 °2θ (±0.2 °2θ; ±0.1 °2θ; or 0.0 °2θ; Cu Kα1 radiation);
(b) an ¹H NMR spectra as provided in FIG. 86;
(c) two or more, or three XRPD signals selected from the group consisting of 10.1 °2θ, 14.0 °2θ, and 15.2 °2θ (±0.2 °2θ; 0.1 °2θ; or 0.0 °2θ; Cu Kα1 radiation); and
(d) two or more, or three XRPD signals selected from the group consisting of 10.1 °2θ, 14.0 °2θ, 15.2 °2θ, 18.2 °2θ, and 23.8 °2θ (±0.2 °2θ; 0.1 °2θ; or 0.0 °2θ; Cu Kα1 radiation).

86.-89. (canceled)

90. The 2C-B malonate salt of claim 4, wherein the salt is a crystalline polymorph, which is characterized by at least one of the characteristics selected from;
(a) two or more, or three XRPD signals selected from the group consisting of 23.2 °2θ, 26.5 °2θ, and 16 °2θ (±0.2 °2θ; ±0.1 °2θ; or 0.0 °2θ; Cu Kα1 radiation);
(b) two or more, or three XRPD signals selected from the group consisting of 5.1 °2θ, 10.2 °2θ, 12.3 °2θ, 9.9 °2θ, and 16 °2θ (±0.2 °2θ; ±0.1 °2θ; or ±0.0 °2θ; Cu Kα1 radiation);
(c) two or more, or three XRPD signals selected from the group consisting of 5.1 °2θ, 10.2 °2θ, and 12.3 °2θ (±0.2 °2θ; ±0.1 °2θ; or 0.0 °2θ; Cu Kα1 radiation).

91. The 2C-B mesylate salt of claim 4, wherein the salt is a crystalline polymorph, which is characterized by at least one of the characteristics selected from;
(a) wo or more, or three XRPD signals selected from the group consisting of 25.9 °2θ, 19.1 °2θ, and 22.5 °2θ (±0.2 °2θ; ±0.1 °2θ; or 0.0 °2θ; Cu Kα1 radiation);
(b) an ¹H NMR spectra as provided in FIG. 87;
(c) an ¹H NMR spectra as provided in FIG. 103;
(d) TGA and DSC profiles as provided in FIG. 118;
(e) TGA and DSC profiles as provided in FIG. 130;
(f) a DVS profile as provided in FIG. 142;
(g) two or more, or three XRPD signals selected from the group consisting of 5.8 °2θ, 11.4 °2θ, and 15.5 °2θ (±0.2 °2θ; ±0.1 °2θ; or ±0.0 °2θ; Cu Kα1 radiation);
(h) two or more, or three XRPD signals selected from the group consisting of 5.8 °2θ, 11.4 °2θ, 15.5 °2θ, 17.6 °2θ, and 19.1 °2θ (±0.2 °2θ; ±0.1 °2θ; or ±0.0 °2θ, Cu Kα1 radiation);
(i) two or more, or three XRPD signals selected from the group consisting of 5.7 °2θ, 11.4 °2θ, and 15.4 °2θ (±0.2 °2θ; ±0.1 °2θ; or ±0.0 °2θ; Cu Kα1 radiation); and
(j) two or more, or three XRPD signals selected from the group consisting of 5.7 °2θ, 11.4 °2θ, 15.4 °2θ, 17.6 °2θ, and 19.1 (±0.2 °2θ, ±0.1 °2θ; or ±0.0 °2θ; Cu Kα1 radiation).

92. (canceled)

93. The 2C-B mucate salt of claim 4, wherein the salt is a crystalline polymorph, which is characterized by at least one of the characteristics selected from;
(a) two or more, or three XRPD signals selected from the group consisting of 15.5 °2θ, 24.9 °2θ, and 9.9 °2θ (±0.2 °2θ; ±0.1 °2θ; or ±0.0 °2θ; Cu Kα1 radiation);
(b) an ¹H NMR spectra as provided in FIG. 88;
(c) two or more, or three XRPD signals selected from the group consisting of 9.9 °2θ, 12.0 °2θ, and 15.5 °2θ (±0.2 °2θ; ±0.1 °2θ; or 0.0 °2θ; Cu Kα1 radiation); and
(d) two or more, or three XRPD signals selected from the group consisting of 9.9 °2θ, 12.0 °2θ, and 15.5 °2θ, 14.8 °2θ, and 19.6 °2θ (±0.2 °2θ; ±0.1 °2θ; or 0.0 °2θ; Cu Kα1 radiation).

94. (canceled)

95. The 2C-B succinate salt of claim 4, wherein the salt is crystalline polymorphic form selected from;
Form 1 and Form 2 plus free succinic acid, which is characterized by at least one of the characteristics selected from;
(a) two or more, or three XRPD signals selected from the group consisting of 17.6 °2θ, 25.9 °2θ, and 25.1 °2θ (±0.2 °2θ; ±0.1 °2θ; or ±0.0 °2θ; Cu Kα1 radiation);
(b two or more, or three XRPD signals selected from the group consisting of 8.2 °2θ, 11.1 °2θ, and 12.5 °2θ (±0.2 °2θ; ±0.1 °2θ; or ±0.0 °2θ; Cu Kα1 radiation); and
(c) two or more, or three XRPD signals selected from the group consisting of 8.2 °2θ, 11.1 °2θ, 12.5 °2θ, 14.2 °2θ and 16.4 °2θ (±0.2 °2θ; ±0.1 °2θ; or 0.0 °2θ; Cu Kα1 radiation); and
Form 1, which is characterized by at least one of the characteristics selected from;
(a) two or more, or three XRPD signals selected from the group consisting of 22.7 °2θ, 26 °2θ, and 17.6 °2θ (±0.2 °2θ; ±0.1 °2θ; or 0.0 °2θ; Cu Kα1 radiation);
(b) an ¹H NMR spectra as provided in FIG. 91;
(c) TGA and DSC profiles as provided in FIG. 120;
(d) two or more, or three XRPD signals selected from the group consisting of 7.6 °2θ, 16.8 °2θ, and 17.6 °2θ (±0.2 °2θ; ±0.1 °2θ; or ±0.0 °2θ; Cu Kα1 radiation); and
(e) two or more, or three XRPD signals selected from the group consisting of 7.6 °2θ, 16.8 °2θ, 17.6 °2θ, 22.7 °2θ, and 23.9 °2θ (±0.2 °2θ; ±0.1 °2θ; or ±0.0 °2θ; Cu Kα1 radiation).

96.-98. (canceled)

99. The 2C-B tartrate salt of claim 4, wherein the salt is crystalline polymorphic form selected from;
Form 1, which is characterized by at least one of the characteristics selected from;
(a) two or more, or three XRPD signals selected from the group consisting of 24.7 °2θ, 20.2 °2θ, and 20.5 °2θ (±0.2 °2θ; ±0.1 °2θ; or 0.0 °2θ; Cu Kα1 radiation);
(b) two or more, or three XRPD signals selected from the group consisting of 20.3 °2θ, 24.7 °2θ, and 27.2 °2θ (±0.2 °2θ; ±0.1 °2θ; or ±0.0 °2θ; Cu Kα1 radiation);
(c) two, three, four, five, six, seven, eight, nine, or ten XRPD signals as shown in Table 70;
(d) two or more, or three XRPD signals as shown in FIG. 64;
(e) an ¹H NMR spectra as provided in FIG. 93;
(f) an ¹H NMR spectra as provided in FIG. 105;
(g) TGA and DSC profiles as provided in FIG. 122;
(h) TGA and DSC profiles as provided in FIG. 132;
(i) a DVS profile as provided in FIG. 144;
(j) exhibit high crystallinity;
(k) exhibit acceptable scale-up;
(l) exhibit minimal water uptake over RH range;
(m) exhibit uncomplicated DSC data (one endothermic event observed);
(n) acceptable solubility when compared to the other salts;
(o) possesses at least one of characteristic as provided in Table 86;
(p) two or more, or three XRPD signals selected from the group consisting of 10.3 °2θ, 12.9 °2θ, and 14.2 °2θ (±0.2 °2θ; ±0.1 °2θ, or ±0.0 °2θ; Cu Kα1 radiation);
(q) two or more, or three XRPD signals selected from the group consisting of 10.3 °2θ, 12.9 °2θ, 14.2 °2θ, 14.4 °2θ, and 20.3 °2θ (±0.2 °2θ; ±0.1 °2θ; or ±0.0 °2θ; Cu Kα1 radiation);
(r) two or more, or three XRPD signals selected from the group consisting of 10.2 °2θ, 12.8 °2θ, and 14.3 °2θ (±0.2 °2θ; ±0.1 °2θ, or 0.0 °2θ; Cu Kα1 radiation);
(s) two or more, or three XRPD signals selected from the group consisting of 10.2 °2θ, 12.8 °2θ, 14.3 °2θ, 14.4 °2θ, and 20.3 °2θ (±0.2 °2θ; ±0.1 °2θ; or ±0.0 °2θ, Cu Kα1 radiation);
(t) two or more, or three XRPD signals selected from the group consisting of 10.2 °2θ, 12.8 °2θ, and 14.2 °2θ (±0.2 °2θ; ±0.1 °2θ; or ±0.0 °2θ; Cu Kα1 radiation);
(u) two or more, or three XRPD signals selected from the group consisting of 10.2 °2θ, 12.8 °2θ, 14.2 °2θ, 14.3 °2θ, and 20.2 °2θ (±0.2 °2θ; ±0.1 °2θ; or ±0.0 °2θ, Cu Kα1 radiation);
(v) two or more, or three XRPD signals selected from the group consisting of 10.2 °2θ, 12.8 °2θ, 14.3 °2θ, 14.4, and 20.3 °2θ (±0.2 °2θ; ±0.1 °2θ, or 0.0 °2θ; Cu Kα1 radiation);
(w) two or more, or three XRPD signals selected from the group consisting of 10.3 °2θ, 12.9 °2θ, 14.2 °2θ, 14.4 °2θ and 20.3 °2θ (±0.2 °2θ; ±0.1 °2θ; or 0.0 °2θ; Cu Kα1 radiation);
(x) prepared by the method provided in Example 5; and
(y) a single crystal structure having unit cell parameters as provided in Table 87A; and
Form 2, which is characterized by at least one of the characteristics selected from;
(a) two or more, or three XRPD signals selected from the group consisting of 22.6 °2θ, 20.9 °2θ, and 11 °2θ (±0.2 °2θ; ±0.1 °2θ; or ±0.0 °2θ, Cu Kα1 radiation);
(b) an ¹H NMR spectra as provided in FIG. 106;
(c) TGA and DSC profiles as provided in FIG. 133;
(d) two or more, or three XRPD signals selected from the group consisting of 11.0 °2θ, 12.4 °2θ, and 13.3 °2θ (±0.2 °2θ; ±0.1 °2θ; or 0.0 °2θ; Cu Kα1 radiation); and
(e) two or more, or three XRPD signals selected from the group consisting of 11.0 °2θ, 12.4 °2θ, 13.3 °2θ, 20.9 °2θ, and 22.6 °2θ (±0.2 °2θ; ±0.1 °2θ; or 0.0 °2θ; Cu Kα1 radiation).

100.-108. (canceled)

109. The 2C-B tartrate salt of claim 4, wherein the salt is anhydrous with 1:1 2C-B:tartaric acid stoichiometry and formula C₁₀H₁₅BrNO₂.C₄H₅O₆.

110.-111. (canceled)

112. The 2C-B tosylate salt of claim 4, wherein the salt is crystalline polymorphic form selected from;
Form 1, which is characterized by at least one of the characteristics selected from;
(a) two or more, or three XRPD signals selected from the group consisting of 20.7 °2θ, 23.3 °2θ, and 18.3 °2θ (±0.2 °2θ; ±0.1 °2θ; or ±0.0 °2θ; Cu Kα1 radiation);
(b) an ¹H NMR spectra as provided in FIG. 94;
(c) TGA and DSC profiles as provided in FIG. 123;
(d) two or more, or three XRPD signals selected from the group consisting of 16.4 °2θ, 18.3 °2θ, and 20.7 °2θ (±0.2 °2θ; ±0.1 °2θ; or ±0.0 °2θ, Cu Kα1 radiation); and
(e) two or more, or three XRPD signals selected from the group consisting of 16.4 °2θ, 18.3 °2θ, 20.7 °2θ, 22.3 °2θ, and 23.3 °2θ (±0.2 °2θ; ±0.1 °2θ; or 0.0 °2θ; Cu Kα1 radiation);
Form 2, which is characterized by at least one of the characteristics selected from;
(a) two or more, or three XRPD signals selected from the group consisting of 8.0 °2θ, 12.6 °2θ, and 13.0 °2θ (±0.2 °2θ; ±0.1 °2θ; or 0.0 °2θ; Cu Kα1 radiation); and
(b) two or more, or three XRPD signals selected from the group consisting of 8.0 °2θ, 12.6 °2θ, 13.0 °2θ, 19.2 °2θ, and 20.7 °2θ (±0.2 °2θ; ±0.1 °2θ; or 0.0 °2θ; Cu Kα1 radiation); and
a mixture of Forms 1 and 2, which is characterized by two or more, or three XRPD signals selected from the group consisting of 23.3 °2θ, 16.4 °2θ, and 20.7 °2θ (±0.2 °2θ; ±0.1 °2θ; or ±0.0 °2θ; Cu Kα1 radiation).

113.-115. (canceled)

116. The 2C-B aspartate salt of claim 4, wherein the salt is crystalline polymorphic form, which is characterized by at least one of the characteristics selected from;
(a) two or more, or three XRPD signals selected from the group consisting of 24.2 °2θ, 16.5 °2θ, and 27.1 °2θ (±0.2 °2θ; ±0.1 °2θ; or ±0.0 °2θ; Cu Kα1 radiation);
(b) two or more, or three XRPD signals selected from the group consisting of 8.2 °2θ, 13.7 °2θ, and 16.5 °2θ (±0.2 °2θ; ±0.1 °2θ; or ±0.0 °2θ, Cu Kα1 radiation); and
(c) two or more, or three XRPD signals selected from the group consisting of 8.2 °2θ, 13.7 °2θ, 16.5 °2θ, 4.1 °2θ, and 22.0 °2θ (±0.2 °2θ; ±0.1 °2θ; or 0.0 °2θ; Cu Kα1 radiation).

117. The 2C-B glutamate salt of claim 4, wherein the salt is crystalline polymorphic form, which is characterized by at least one of the characteristics selected from;
(a) two or more, or three XRPD signals selected from the group consisting of 3.7 °2θ, 7.3 °2θ, and 21.9 °2θ (±0.2 °2θ; ±0.1 °2θ, or ±0.0 °2θ; Cu Kα1 radiation);
(b) two or more, or three XRPD signals selected from the group consisting of 21.9 °2θ, 3.7 °2θ, and 7.3 °2θ (±0.2 °2θ; ±0.1 °2θ; or 0.0 °2θ; Cu Kα1 radiation);
(c) two or more, or three XRPD signals selected from the group consisting of 21.9 °2θ, 3.7 °2θ, 7.3 °2θ, 14.5 °2θ, and 16.4 °2θ (±0.2 °2θ; ±0.1 °2θ; or 0.0 °2θ; Cu Kα1 radiation);
(d) two or more, or three XRPD signals selected from the group consisting of 22 °2θ, 25.5 °2θ, and 3.6 °2θ (±0.2 °2θ; ±0.1 °2θ; or ±0.0 °2θ; Cu Kα1 radiation);
(e) an ¹H NMR spectra as provided in FIG. 80;
(f) TGA and DSC profiles as provided in FIG. 114;
(g) two or more, or three XRPD signals selected from the group consisting of 3.6 °2θ, 7.3 °2θ, and 22.0 °2θ (±0.2 °2θ; ±0.1 °2θ; or 0.0 °2θ; Cu Kα1 radiation); and
(h) two or more, or three XRPD signals selected from the group consisting of 3.6 °2θ, 7.3 °2θ, 22.0 °2θ, 14.6 °2θ, and 16.4 °2θ (±0.2 °2θ; ±0.1 °2θ; or 0.0 °2θ; Cu Kα1 radiation).

118.-120. (canceled)

121. The 2C-B salt of claim 4, wherein in the salt has at least one improved property compared to amorphous 2C-B salt.

122. A pharmaceutical composition, comprising the salt of claim 4, and a pharmaceutically acceptable excipient.

123. A method of treatment or prevention of psychiatric disorder, neurological disorder, or both, comprising administering to a subject an effective amount of a salt or solid form of 2C-B, or the pharmaceutical composition of claim 122.

124.-131. (canceled)

132. The method of claim 123, further comprising administering to the subject an effective amount of an empathogenic agent.

133.-135. (canceled)

136. The 2C-B salt of claim 4, wherein the salt is 2C-B tartrate crystalline polymorphic form (Form 1) and wherein the salt is obtained by;
(i) adding L-tartaric acid to a solution of 2C-B free base in ethanol, resulting in precipitation,
(ii) adding more solvent to form a suspension,
(iii) slurring the suspension at room temperature for about 5 days, and
(iv) isolating solids via filtration and drying under vacuum at room temperature for about 1 day.

137. An acid salt of 4-bromo-2,5-dimethoxyphenethylamine, wherein the salt has a molar ratio of acid to 4-bromo-2,5-dimethoxyphenethylamine selected from the groups consisting of;
(a) from about 0.4 molar equivalents to about 2.2 molar equivalents,
(b) from about 0.5 molar equivalents to about 2 molar equivalents
(c) about 0.5 molar equivalents, about 1 molar equivalent, and
(d) about 2 molar equivalents of the acid.

138.-141. (canceled)

142. The 2C-B salt of claim 4, wherein the salt possesses at least one of the following characteristics;
(a) exhibit high crystallinity,
(b) exhibit acceptable scale-up
(c) exhibit minimal water uptake over RH range,
(d) exhibit uncomplicated DSC data (one endothermic event observed), and
(e) acceptable solubility when compared to the other salts.

143.-150. (canceled)

151. The 2C-B salt of claim 4, wherein the salt is 2C-B tartrate crystalline polymorphic form (Form 1) and wherein the salt is obtained by;
(i) adding L-tartaric acid to a solution of 2C-B free base in acetone, resulting in gel,
(ii) adding more solvent and heating at about 50° C. with stirring until the gel solidifies,
(iii) slurring the solidified gel at elevated temperature for about 4 days,
(iv) cooling to room temperature and stirring for about 6 days to form a product and mother liquor,
(v) centrifuging the product and mother liquor,
(vi) removing the mother liquor, and
(vii) isolating solids and drying.

152. The 2C-B salt of claim 4, wherein the salt is 2C-B tartrate crystalline polymorphic form (Form 2) and wherein the salt is obtained by;
(i) adding L-tartaric acid to a solution of 2C-B free base in ethanol to form a mixture,
(ii) sonicating the mixture to form a solid plug,
(iii) adding ethanol to form a suspension,
(iv) slurring the suspension at room temperature for about 4 days, and
(v) isolating solids via filtration and drying under vacuum at room temperature.

153.-203. (canceled)

204. A mescaline.HCl, wherein the salt is a crystalline polymorph characterized by:
(a) two or more, or three XRPD signals selected from the group consisting of 9.4 °2θ, 13.8 °2θ, and 14.2 °2θ (±0.2 °2θ; ±0.1 °2θ; or 0.0 °2θ; Cu Kα1 radiation); or
(b) two or more, or three XRPD signals selected from the group consisting of 9.4 °2θ, 13.8 °2θ, 14.2 °2θ, 15.8 °2θ, and 19.0 °2θ (±0.2 °2θ; ±0.1 °2θ; or 0.0 °2θ; Cu Kα1 radiation).

205.-237. (canceled)

238. The solid form of 2C-B of claim 1, wherein the solid form is a crystalline polymorph form selected from;
a mixture of free base Forms 1 and 2, which is characterized by at least one of the characteristics selected from;
(a) two or more, or three XRPD signals selected from the group consisting of 9.2 °2θ, 17 °2θ, and 18.6 °2θ (±0.2 °2θ; ±0.1 °2θ; or 0.0 °2θ; Cu Kα1 radiation); and
(b) two or more, or three XRPD signals selected from the group consisting of 9.2 °2θ, 17 °2θ, 18.6 °2θ, 23.6 °2θ, and 24.3 °2θ (±0.2 °2θ; ±0.1 °2θ; or ±0.0 °2θ Cu Kα1 radiation); and
free base Form 1, which is characterized by at least one of the characteristics selected from:
(a) two or more, or three XRPD signals selected from the group consisting of 5.3 °2θ, 10.5 °2θ, and 15.3 °2θ (±0.2 °2θ; 0.1 °2θ; or ±0.0 °2θ; Cu Kα1 radiation); and
(b) two or more, or three XRPD signals selected from the group consisting of 5.3 °2θ, 10.5 °2θ, and 15.3 °2θ, 16 °2θ, and 17.6 (±0.2 °2θ; 0.1 °2θ; or 0.0 °2θ; Cu Kα1 radiation).

239.-258. (canceled)
